(19) United States
(12) Patent Application Publication
(10) Pub. No.: US 2024/0122941 A1
(43) Pub. Date: Apr. 18, 2024

(54) THERAPY BASED ON SYNTHETIC LETHALITY IN SWI/SNF COMPLEX-DYSFUNCTION CANCER

(71) Applicants: NATIONAL CANCER CENTER, Tokyo (JP); Sumitomo Pharma Co., Ltd., Osaka-shi (JP)

(72) Inventors: Hideaki OGIWARA, Chuo-ku (JP); Mariko SASAKI, Chuo-ku (JP)

(73) Assignees: NATIONAL CANCER CENTER, Tokyo (JP); Sumitomo Pharma Co., Ltd., Osaka-shi (JP)

(21) Appl. No.: 18/258,989

(22) PCT Filed: Dec. 24, 2021
(86) PCT No.: PCT/JP2021/048330

§ 371 Date: Jun. 22, 2023

(30) Foreign Application Priority Data
Dec. 25, 2020 (JP) 2020-217707
Oct. 29, 2021 (JP) 2021-177849

(51) Int. Cl. A61K 31/553; A61K 31/405; A61K 31/415; A61K 31/4172; A61K 31/4178; A61K 31/4184; A61K 31/421; A61K 31/426; A61K 31/431; A61K 31/437; A61K 31/4375; A61K 31/4439; A61K 31/4725; A61K 31/496; A61K 31/497; A61K 31/501; A61K 31/506; A61K 31/675; A61P 35/00; C12N 9/10; C12N 9/99

(52) U.S. Cl. CPC A61K 31/553; A61K 31/405; A61K 31/415; A61K 31/4172; A61K 31/4178; A61K 31/4184; A61K 31/421; A61K 31/426; A61K 31/431; A61K 31/437; A61K 31/4375; A61K 31/4439; A61K 31/4725; A61K 31/496; A61K 31/497; A61K 31/501; A61K 31/506; A61K 31/675; A61P 35/00; C12N 9/1025; C12N 9/99

(57) ABSTRACT

The present disclosure provides a pharmaceutical composition for treating and/or preventing SWI/SNF complex-dysfunction cancer. More specifically, according to the present disclosure, a compound represented by formulae (1) to (23) (the formulae are as set forth in the specification) or a pharmaceutically acceptable salt thereof can have a therapeutic and/or prophylactic effect on SWI/SNF complex-dysfunction cancer. A pharmaceutical composition, which is for treating and/or preventing cancer and contains a CBP/P300 inhibitor, can be provided. The cancer may be SWI/SNF complex-dysfunction cancer. The SWI/SNF complex-dysfunction cancer may be BAF complex-dysfunction cancer. The BAF complex-dysfunction cancer may be SMARC-deficient cancer, ARID-deficient cancer, or SS18-SSX fusion cancer. The SMARC-deficient cancer may be SMARCB1-deficient cancer, SMARCA2-deficient cancer, SMARCA4-deficient cancer, or SMARCA2/A4-deficient cancer.

### TECHNICAL FIELD

The present disclosure relates to a pharmaceutical composition for treating and/or preventing SWI/SNF complex dysfunction cancer.

### BACKGROUND ART

SWI/SNF complexes are involved in various cellular processes such as differentiation and growth by mediating ATP dependent chromatin remodeling and regulating gene expression and DNA repair. SWI/SNF complexes are largely classified into three types of complexes with different constituent elements (BAF complex, PBAF complex, and ncBAF complex). Many studies report that a mutation in a gene encoding a constituent element of an SWI/SNF complex is involved in malignant transformation (Non Patent Literature 1). In particular, genetic mutations of SMARCB1/INI1/SNF5/BAF47, SMARCA2/BAF190/BIS/BRM/NCBRS/SNF2/SNF2LA/SNF2L2, SMARCA4/BAF190A/BRG1/CSS4/MRD16/RTPS2/SNF2/SNF2B/SNF2L4/SNF 2LB, ARID1A/B120/BAF250/BAF250a/BM029/C1orf4/CSS2/ELD/MRD14/OSA1/P270/SMARCF1/hELD/hOSA1, ARID1B/6A3-5/BAF250B/BRIGHT/CSS1/DAN15/ELD/OSA1/MRD12/OSA2/P250R/SMARC F2, and SS18/SMARCL1/SSXT/SYT are reported in multiple cancer species (Non Patent Literatures 2, 3, and 4).

For example, malignant rhabdoid tumor is tumor with very poor prognosis which occurs in any part of the body, particularly in the kidney, central nervous system, soft tissue, etc. In almost all cases, loss of function of SMARCB1 is found. As a method of treating malignant rhabdoid tumor, a therapy combining surgery, polypharmaceutic chemotherapy, and radiation therapy is administered, but the therapeutic outcome thereof is not sufficient. An effective therapeutic method has yet to be established. A certain number of instances of loss of function (suppression of function) of SMARCA2, SMARCA4, or SMARCA2/A4 is found in various cancers including pulmonary adenocarcinoma, a certain number of instances of loss of function (suppression of function) of ARID1A, ARID1B, or ARID1A/1B is found in ovarian cancer and colon cancer, and a certain number of instances of fusion of SS18 and SSX is found in synovial sarcoma and Ewing's sarcoma. Meanwhile, an effective therapeutic method for cancer associated with dysfunction of these agents have yet to be established.

If a function of a gene is lost, survival of a cell would be dependent on the function of another specific gene. A phenomenon where a cell dies when the function of this gene is inhibited is known as “synthetic lethality”. Cancer therapeutic method utilizing “synthetic lethality” (synthetic lethality therapeutic method) is expected as a novel approach to cancer therapeutic method (Non Patent Literature 5).

Histone acetyltransferase CBP/CREBBP and P300/EP300 acetylate a histone protein, resulting in chromatin to be in an open state and promoting expression of a proximal gene (Non Patent Literature 6). While it was known that inhibition of CBP and P300 suppresses proliferative activity of cells (Non Patent Literature 7), there was no disclosure or suggestion that this would be useful as a synthetic lethality therapeutic method for SWI/SNF complex dysfunction cancer.

### CITATION LIST

### Non Patent Literature

[NPL 1] Oncogene. 2009 April; 28(14): 1653-1668
[NPL 2] Cancer Sci. 2017 April; 108(4): 547-552
[NPL 3] Ann Diagn Pathol. 2017 February; 26: 47-51
[NPL 4] Am J Med Genet C Semin Med Genet. 2014 September; 0(3): 350-366
[NPL 5] Nat Rev Drug Discov. 2020 January; 19(1): 22-38
[NPL 6] Cell Mol Life Sci. 2013 November; 70(21): 3989-4008
[NPL 7] Endocr Relat Cancer. 2020 March; 27(3): 187-198

### SUMMARY OF INVENTION

### Solution to Problem

The present disclosure provides a pharmaceutical composition for treating and/or preventing SWI/SNF complex dysfunction cancer, comprising a CBP/P300 inhibitor.

As a result of diligent studies, the inventors have discovered that a combination of “CBP/P300 inhibition” and “SWI/SNF complex dysfunction” exhibits synthetic lethality. Specifically, it was discovered that a CBP/P300 inhibitor exhibits a significant effect of suppressing growth on SMARCB1 deficient cancer including malignant rhabdoid tumor. It was discovered that a CBP/P300 inhibitor also exhibits a significant effect of suppressing growth of SMARCA2/A4 deficient cancer and SMARCA4 deficient cancer including pulmonary adenocarcinoma. Furthermore, it was discovered that a CBP/P300 inhibitor exhibits a significant effect of suppressing growth of ARID1A/1B deficient cancer and ARID1A deficient cancer including ovarian cancer and SS18-SSX fusion cancer including synovial sarcoma.

More specifically, the inventors discovered that growth of cancer cells was suppressed significantly when a HAT inhibitor that inhibits a HAT domain or a BRD inhibitor that inhibits a BRD domain, which can inhibit the function of CBP/P300, was applied to SMARCB1 deficient cancer cells including malignant rhabdoid tumor, SMARCA2/A4 deficient cancer cells including pulmonary adenocarcinoma, ARID1A/1B or ARID1A deficient cancer cells including ovarian cancer, and cancer cells accompanied by SS18-SSX fusion including synovial sarcoma. The inventors also discovered that growth of cancer cells was suppressed significantly when a HAT inhibitor that inhibits a HAT domain, which can inhibit the function of CBP/P300, was applied to SMARCA4 deficient cancer cells. Furthermore, growth of the cancer cells was suppressed significantly when expression of CBP/P300 was selectively suppressed using siRNA. These results revealed that a combination of CBP/P300 and SWI/SNF complex exhibits synthetic lethality.

Specifically, the present disclosure includes the following.

### [Item 1]

A pharmaceutical composition for use in treating and/or preventing cancer, comprising a CBP/P300 inhibitor.

### [Item 2]

The pharmaceutical composition of item 1, wherein the cancer is SWI/SNF complex dysfunction cancer.

### [Item 3]

The pharmaceutical composition of item 2, wherein the SWI/SNF complex dysfunction cancer is BAF complex dysfunction cancer.

### [Item 4]

The pharmaceutical composition of item 3, wherein the BAF complex dysfunction cancer comprises at least one selected from the group consisting of SMARC deficient cancer, SS18-SSX fusion cancer, and ARID deficient cancer.

### [Item 5]

The pharmaceutical composition of item 1, wherein the cancer is SMARC deficient cancer.

### [Item 6]

The pharmaceutical composition of item 5, wherein the SMARC deficient cancer is cancer deficient of at least one agent selected from the group consisting of SMARCB1, SMARCA2, and SMARCA4.

### [Item 7]

The pharmaceutical composition of item 5, wherein the SMARC deficient cancer comprises at least one selected from the group consisting of SMARCB1 deficient cancer, SMARCA2 deficient cancer, SMARCA4 deficient cancer, and SMARCA2/A4 deficient cancer.

### [Item 8]

The pharmaceutical composition of item 5, wherein the SMARC deficient cancer is SMARCB1 deficient cancer.

### [Item 9]

The pharmaceutical composition of item 8, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

### [Item 10]

The pharmaceutical composition of item 8, wherein the SMARCB1 deficient cancer comprises at least one selected from the group consisting of malignant rhabdoid tumor, epithelioid sarcoma, and atypical teratoid/rhabdoid tumor.

### [Item 11]

The pharmaceutical composition of item 8, wherein the SMARCB1 deficient cancer is malignant rhabdoid tumor.

### [Item 12]

The pharmaceutical composition of item 5, wherein the SMARC deficient cancer is SMARCA2 deficient cancer.

### [Item 13]

The pharmaceutical composition of item 12, wherein the SMARCA2 deficient cancer comprises at least one selected from the group consisting of pulmonary adenocarcinoma, large cell lung carcinoma, lung neuroendocrine tumor, esophageal cancer, gastroesophageal junction cancer, and malignant rhabdoid tumor.

### [Item 14]

The pharmaceutical composition of item 12, wherein the SMARCA2 deficient cancer is pulmonary adenocarcinoma.

### [Item 15]

The pharmaceutical composition of item 5, wherein the SMARC deficient cancer is SMARCA4 deficient cancer.

### [Item 16]

The pharmaceutical composition of item 15, wherein the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

### [Item 17]

The pharmaceutical composition of item 15, wherein the SMARCA4 deficient cancer is pulmonary adenocarcinoma.

### [Item 18]

The pharmaceutical composition of item 5, wherein the SMARC deficient cancer is SMARCA2/A4 deficient cancer.

### [Item 19]

The pharmaceutical composition of item 18, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

### [Item 20]

The pharmaceutical composition of item 18, wherein the SMARCA2/A4 deficient cancer is pulmonary adenocarcinoma.

### [Item 21]

The pharmaceutical composition of item 1, wherein the cancer is ARID deficient cancer.

### [Item 22]

The pharmaceutical composition of item 21, wherein the ARID deficient cancer is cancer deficient of at least one agent selected from the group consisting of ARID1A and ARID1B.

### [Item 23]

The pharmaceutical composition of item 21, wherein the ARID deficient cancer comprises at least one selected from the group consisting of ARID1A deficient cancer, ARID1B deficient cancer, and ARID1A/1B deficient cancer.

### [Item 24]

The pharmaceutical composition of item 21, wherein the ARID deficient cancer is ARID1A deficient cancer.

### [Item 25]

The pharmaceutical composition of item 24, wherein the ARID1A deficient cancer comprises at least one selected from the group consisting of ovarian cancer, gastric cancer, bile duct cancer, pancreatic cancer, uterine cancer, neuroblastoma, colon cancer, and bladder cancer.

### [Item 26]

The pharmaceutical composition of item 24, wherein the ARID1A deficient cancer is ovarian cancer.

### [Item 27]

The pharmaceutical composition of item 21, wherein the ARID deficient cancer is ARID1B deficient cancer.

### [Item 28]

The pharmaceutical composition of item 27, wherein the ARID1B deficient cancer comprises at least one selected from the group consisting of ovarian cancer, colon cancer, pancreatic cancer, liver cancer, melanoma, breast cancer, medulloblastoma, uterine cancer, bladder cancer, and gastric cancer.

### [Item 29]

The pharmaceutical composition of item 21, wherein the ARID1B deficient cancer is ovarian cancer.

### [Item 30]

The pharmaceutical composition of item 21, wherein the ARID deficient cancer is ARID1A/1B deficient cancer.

### [Item 31]

The pharmaceutical composition of item 30, wherein the ARID1A/1B deficient cancer comprises at least one selected from the group consisting of ovarian cancer, colon cancer, uterine cancer, neuroblastoma, bladder cancer, and gastric cancer.

### [Item 32]

The pharmaceutical composition of item 30, wherein the ARID1A/1B deficient cancer is ovarian cancer.

### [Item 33]

The pharmaceutical composition of item 1, wherein the cancer is SS18-SSX fusion cancer.

### [Item 34]

The pharmaceutical composition of item 33, wherein the SS18-SSX fusion cancer is synovial sarcoma or Ewing's sarcoma.

### [Item 35]

The pharmaceutical composition of item 33, wherein the SS18-SSX fusion cancer is synovial sarcoma.

### [Item 36]

The pharmaceutical composition of any one of items 1 to 35, wherein the CBP/P300 inhibitor is a HAT inhibitor, a BRD inhibitor, an antisense nucleic acid for a transcriptional product of a gene encoding CBP or P300, a ribozyme for a transcriptional product of a gene encoding CBP or P300, or a nucleic acid having RNAi activity for a transcriptional product of a gene encoding CBP or P300, or a precursor thereof.

### [Item 37]

The pharmaceutical composition of item 36, wherein the CBP/P300 inhibitor is a HAT inhibitor or a BRD inhibitor.

### [Item 38]

The pharmaceutical composition of item 37, wherein the CBP/P300 inhibitor is a HAT inhibitor.

### [Item 39]

The pharmaceutical composition of any one of items 36 to 38, wherein activity of the HAT inhibitor inhibits histone acetyltransferase (HAT) activity of CBP and/or P300 by 50% or more at 20 μM.

### [Item 40]

The pharmaceutical composition of any one of items 36 to 38, wherein activity of the HAT inhibitor inhibits histone acetyltransferase (HAT) activity of CBP and/or P300 by 80% or more at 20 μM.

### [Item 41]

The pharmaceutical composition of any one of items 1 to 40, wherein the CBP/P300 inhibitor is a nucleic acid or a low molecular weight compound.

### [Item 42]

The pharmaceutical composition of any one of items 36 to 41, wherein the HAT inhibitor is a low molecular weight compound.

### [Item 43]

The pharmaceutical composition of item 42, wherein the low molecular weight compound is a compound represented by formula (1)

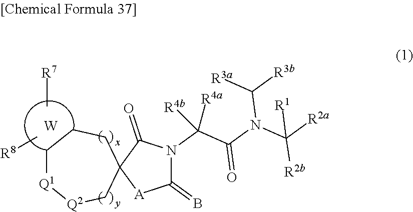

wherein
Q¹ - - - Q² is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or —C(R¹⁰)═C(R¹⁴)—;
A is —NR⁸—, —O—, or —S—;
B is O or NH;
W is arylene or heteroarylene;
R¹ is carbocyclyl or heterocyclyl;
R^{2a} and R^{2b} are each independently a hydrogen atom, a deuterium atom, C₁₋₆ alkyl, C₁₋₆ alkenyl, or C₁₋₆ alkynyl;
R^{3a} is a hydrogen atom, C(O)NH₂, C₁₋₆ alkyl, C₁₋₆ alkenyl, C₁₋₆ alkynyl, aryl, cycloalkyl, or heterocyclyl;
R^{3b} is C₁₋₆ alkyl, C₁₋₆ alkenyl, C₁₋₆ alkynyl, aryl, cycloalkyl, or heterocyclyl; or
wherein R^{3a} and R^{3b}, together with the carbon atom to which they are attached, may form arene, cycloalkane, or heterocyclyl;
R^{4a} and R^{4b} are each independently a hydrogen atom, a deuterium atom, C₁₋₆ alkyl, C₁₋₆ alkenyl, or C₁₋₆ alkynyl;
R⁶ and R⁷ are each independently a hydrogen atom, a halogen atom, —OH, —CN, —CO₂H, C₁₋₆ alkyl, C₁₋₆ alkenyl, C₁₋₆ alkynyl, alkoxy, haloalkoxy, alkoxyalkyl, haloalkoxyalkyl, hydroxyalkyl, hydroxyalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, NHC(═O)NR¹²S(O)₂OR¹², —NHC(═O)NR¹²S(O)₂N(R¹²)₂, —NHC(═S) N(R¹²)₂, —NHC(═N—C≡N) NR¹², —NHC(═N—C≡N) SR¹², or —NHS(O)ₘR¹²;
R⁸ and R⁹ are each independently a hydrogen atom, C₁₋₆ alkyl, C₁₋₆ alkenyl, or C₁₋₆ alkynyl;
R¹⁰ is, for each instance, each independently a hydrogen atom, —OH, a halogen atom, —CN, —CO₂R¹², —C(═O)NHR¹³, —NHR¹², C₁₋₆ alkyl, C₁₋₆ alkenyl, C₁₋₆ alkynyl, or alkoxy; or wherein two R¹⁰ together may form oxo or ═N—OR¹¹;
R¹¹ and R¹³ are each independently a hydrogen atom, —OH, C₁₋₆ alkyl, C₁₋₆ alkenyl, or C₁₋₆ alkynyl;
R¹² is, for each instance, each independently a hydrogen atom, C₁₋₆ alkyl, C₁₋₆ alkenyl, C₁₋₆ alkynyl, aryl, cycloalkyl, or heterocyclyl;
R¹⁴ is, for each instance, each independently a hydrogen atom, C₁₋₆ alkyl, C₁₋₆ alkenyl, or C₁₋₆ alkynyl;
m is, for each instance, each independently 0, 1, or 2;
x and y are each independently 0 or 1, wherein x and y are chosen so that the sum of x+y is 0 or 1;
with the proviso that if R¹ and W are each unsubstituted phenyl, A is —NH, x is 0 or 1, y is 0, and Q¹ - - - Q² is —C(R¹⁰)₂—C(R¹⁴)₂—, each of R^{3a} and R^{3b} is not cyclopropyl or methyl; and
if at least one of R¹ and W is unsubstituted phenyl, and A is —NH, R^{3a} and R^{3b}, together with the carbon atom to which they are attached, do not form tetrahydrothiophene 1,1-dioxide or tetrahydrothiophene;
   or a prodrug thereof or a pharmaceutically acceptable salt thereof.

### [Item 44]

The pharmaceutical composition of item 42, wherein the low molecular weight compound is compound represented by the following (Table 1)

TABLE 22

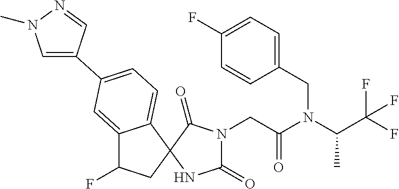 [SMILES: C[C@H](N(Cc1ccc(F)cc1)C(=O)CN1C(=O)NC2(CC(F)c3cc(-c4cnn(C)c4)ccc32)C1=O)C(F)(F)F]

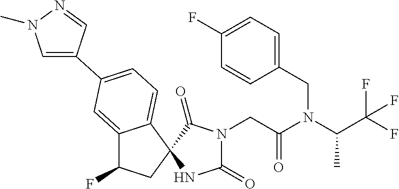 [SMILES: C[C@H](N(Cc1ccc(F)cc1)C(=O)CN1C(=O)N[C@]2(C[C@@H](F)c3cc(-c4cnn(C)c4)ccc32)C1=O)C(F)(F)F]

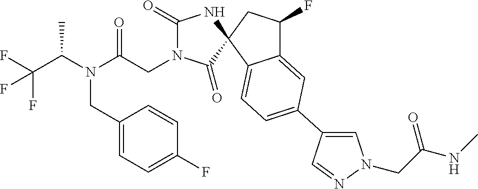 [SMILES: CNC(=O)Cn1cc(-c2ccc3c(c2)[C@H](F)C[C@]32NC(=O)N(CC(=O)N(Cc3ccc(F)cc3)[C@@H](C)C(F)(F)F)C2=O)cn1]

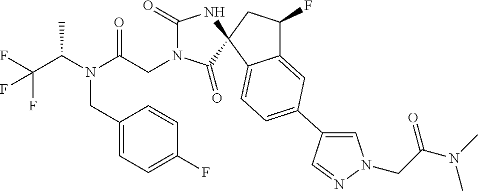 [SMILES: C[C@H](N(Cc1ccc(F)cc1)C(=O)CN1C(=O)N[C@]2(C[C@@H](F)c3cc(-c4cnn(CC(=O)N(C)C)c4)ccc32)C1=O)C(F)(F)F]

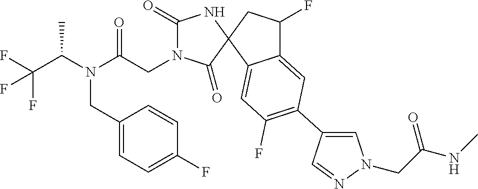 [SMILES: CNC(=O)Cn1cc(-c2cc3c(cc2F)C2(CC3F)NC(=O)N(CC(=O)N(Cc3ccc(F)cc3)[C@@H](C)C(F)(F)F)C2=O)cn1]

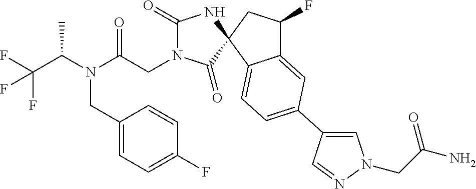 [SMILES: C[C@H](N(Cc1ccc(F)cc1)C(=O)CN1C(=O)N[C@]2(C[C@@H](F)c3cc(-c4cnn(CC(N)=O)c4)ccc32)C1=O)C(F)(F)F]

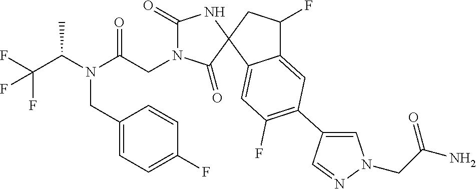 [SMILES: C[C@H](N(Cc1ccc(F)cc1)C(=O)CN1C(=O)NC2(CC(F)c3cc(-c4cnn(CC(N)=O)c4)c(F)cc32)C1=O)C(F)(F)F]

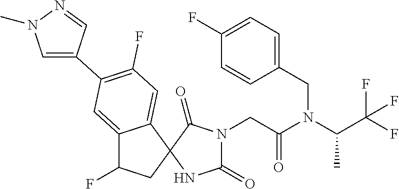 [SMILES: C[C@H](N(Cc1ccc(F)cc1)C(=O)CN1C(=O)NC2(CC(F)c3cc(-c4cnn(C)c4)c(F)cc32)C1=O)C(F)(F)F]

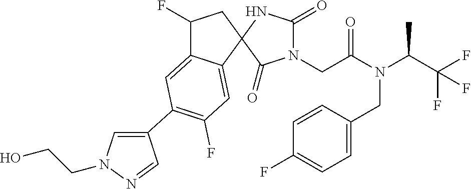 [SMILES: C[C@H](N(Cc1ccc(F)cc1)C(=O)CN1C(=O)NC2(CC(F)c3cc(-c4cnn(CCO)c4)c(F)cc32)C1=O)C(F)(F)F]

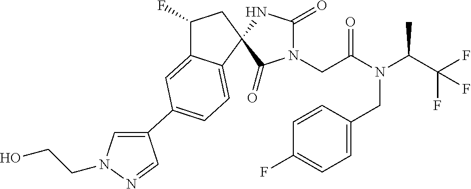 [SMILES: C[C@H](N(Cc1ccc(F)cc1)C(=O)CN1C(=O)N[C@]2(C[C@@H](F)c3cc(-c4cnn(CCO)c4)ccc32)C1=O)C(F)(F)F]

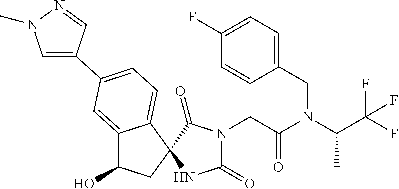 [SMILES: C[C@H](N(Cc1ccc(F)cc1)C(=O)CN1C(=O)N[C@]2(C[C@@H](O)c3cc(-c4cnn(C)c4)ccc32)C1=O)C(F)(F)F]

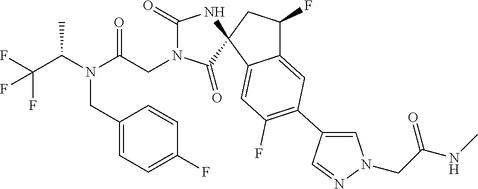 [SMILES: CNC(=O)Cn1cc(-c2cc3c(cc2F)[C@]2(C[C@H]3F)NC(=O)N(CC(=O)N(Cc3ccc(F)cc3)[C@@H](C)C(F)(F)F)C2=O)cn1]

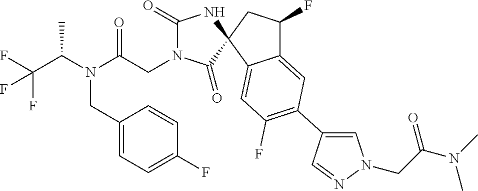 [SMILES: C[C@H](N(Cc1ccc(F)cc1)C(=O)CN1C(=O)N[C@]2(C[C@@H](F)c3cc(-c4cnn(CC(=O)N(C)C)c4)c(F)cc32)C1=O)C(F)(F)F]

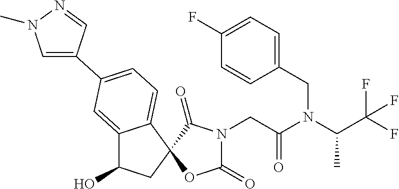 [SMILES: C[C@H](N(Cc1ccc(F)cc1)C(=O)CN1C(=O)O[C@]2(C[C@@H](O)c3cc(-c4cnn(C)c4)ccc32)C1=O)C(F)(F)F]

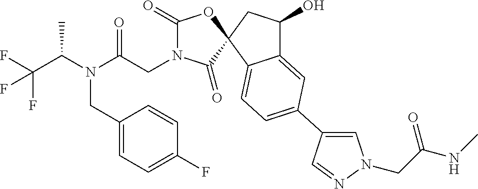 [SMILES: CNC(=O)Cn1cc(-c2ccc3c(c2)[C@H](O)C[C@]32OC(=O)N(CC(=O)N(Cc3ccc(F)cc3)[C@@H](C)C(F)(F)F)C2=O)cn1]

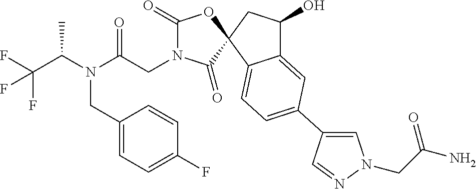 [SMILES: C[C@H](N(Cc1ccc(F)cc1)C(=O)CN1C(=O)O[C@]2(C[C@@H](O)c3cc(-c4cnn(CC(N)=O)c4)ccc32)C1=O)C(F)(F)F]

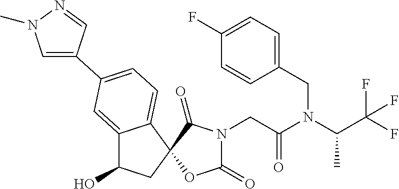 [SMILES: C[C@H](N(Cc1ccc(F)cc1)C(=O)CN1C(=O)O[C@@]2(C[C@@H](O)c3cc(-c4cnn(C)c4)ccc32)C1=O)C(F)(F)F]

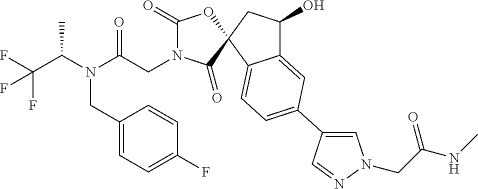 [SMILES: CNC(=O)Cn1cc(-c2ccc3c(c2)[C@H](O)C[C@@]32OC(=O)N(CC(=O)N(Cc3ccc(F)cc3)[C@@H](C)C(F)(F)F)C2=O)cn1]

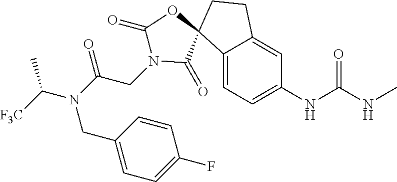 [SMILES: CNC(=O)Nc1ccc2c(c1)CC[C@]21OC(=O)N(CC(=O)N(Cc2ccc(F)cc2)[C@@H](C)C(F)(F)F)C1=O]

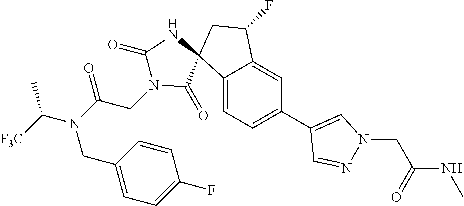 [SMILES: CNC(=O)Cn1cc(-c2ccc3c(c2)[C@@H](F)C[C@]32NC(=O)N(CC(=O)N(Cc3ccc(F)cc3)[C@@H](C)C(F)(F)F)C2=O)cn1]

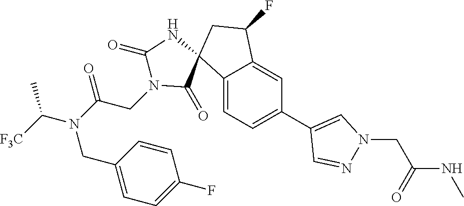 [SMILES: CNC(=O)Cn1cc(-c2ccc3c(c2)[C@H](F)C[C@@]32NC(=O)N(CC(=O)N(Cc3ccc(F)cc3)[C@@H](C)C(F)(F)F)C2=O)cn1]

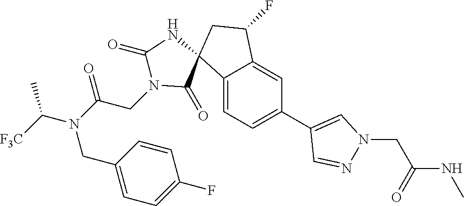 [SMILES: CNC(=O)Cn1cc(-c2ccc3c(c2)[C@@H](F)C[C@@]32NC(=O)N(CC(=O)N(Cc3ccc(F)cc3)[C@@H](C)C(F)(F)F)C2=O)cn1]

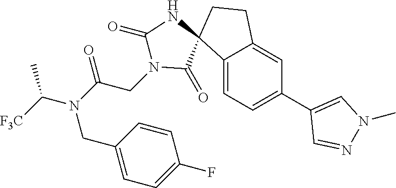 [SMILES: C[C@H](N(Cc1ccc(F)cc1)C(=O)CN1C(=O)N[C@]2(CCc3cc(-c4cnn(C)c4)ccc32)C1=O)C(F)(F)F]

or a pharmaceutically acceptable salt thereof.

### [Item 45]

The pharmaceutical composition of item 42, wherein the low molecular weight compound is a compound represented by formula (2)

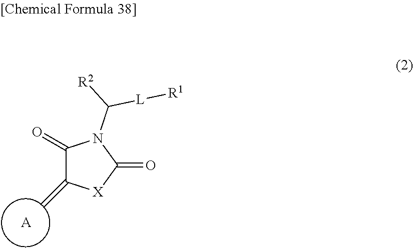

wherein
A is carbocyclyl or heterocyclyl with a 6-, 7-, or 8-membered ring, and heterocyclyl is comprised of a carbon atom, and one or more heteroatoms selected from O and S;
X is —S— or —NH—;
L is a direct bond or a linker;
R¹ is aryl, heteroaryl, or cycloalkyl;
R² is a hydrogen atom, a deuterium atom, C₁₋₆ alkyl, C₁₋₆ alkenyl, or C₁₋₆ alkynyl;
with the proviso that if A is unsubstituted cyclohexyl, R² is a hydrogen atom, and X is —S—, R¹ is not p-aminosulfonylphenyl or p-fluorophenyl,
   or a prodrug thereof or a pharmaceutically acceptable salt thereof.

### [Item 46]

The pharmaceutical composition of item 42, wherein the low molecular weight compound is a compound represented by the following (Table 2)

TABLE 23-1

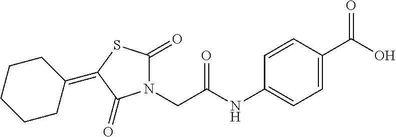 [SMILES: O=C(CN1C(=O)SC(=C2CCCCC2)C1=O)Nc1ccc(C(=O)O)cc1]

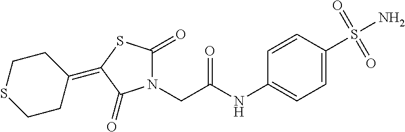 [SMILES: NS(=O)(=O)c1ccc(NC(=O)CN2C(=O)SC(=C3CCSCC3)C2=O)cc1]

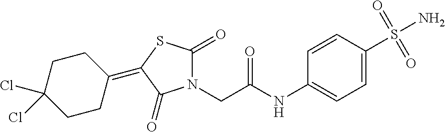 [SMILES: NS(=O)(=O)c1ccc(NC(=O)CN2C(=O)SC(=C3CCC(Cl)(Cl)CC3)C2=O)cc1]

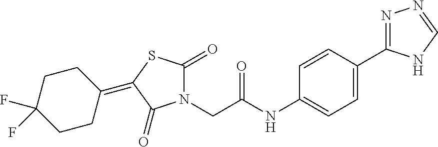 [SMILES: O=C(CN1C(=O)SC(=C2CCC(F)(F)CC2)C1=O)Nc1ccc(-c2nnc[nH]2)cc1]

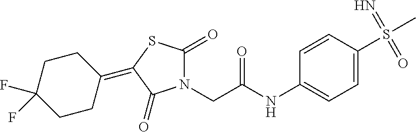 [SMILES: CS(=N)(=O)c1ccc(NC(=O)CN2C(=O)SC(=C3CCC(F)(F)CC3)C2=O)cc1]

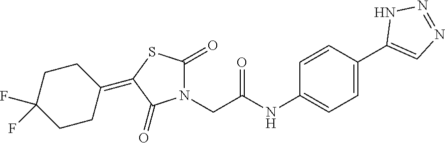 [SMILES: O=C(CN1C(=O)SC(=C2CCC(F)(F)CC2)C1=O)Nc1ccc(-c2cnn[nH]2)cc1]

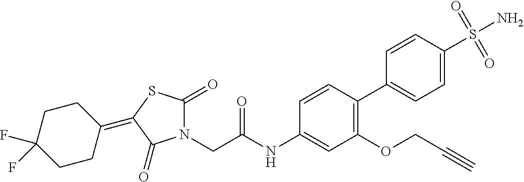 [SMILES: C#CCOc1cc(NC(=O)CN2C(=O)SC(=C3CCC(F)(F)CC3)C2=O)ccc1-c1ccc(S(N)(=O)=O)cc1]

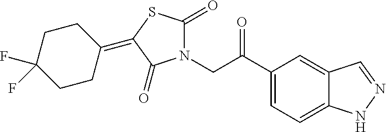 [SMILES: O=C(CN1C(=O)SC(=C2CCC(F)(F)CC2)C1=O)c1ccc2[nH]ncc2c1]

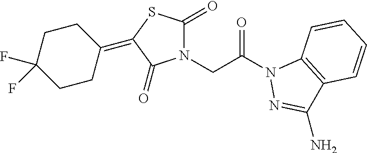 [SMILES: Nc1nn(C(=O)CN2C(=O)SC(=C3CCC(F)(F)CC3)C2=O)c2ccccc12]

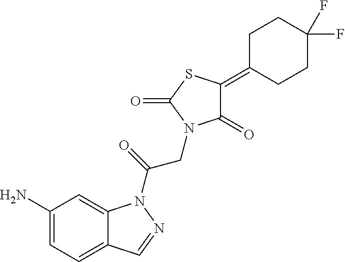 [SMILES: Nc1ccc2cnn(C(=O)CN3C(=O)SC(=C4CCC(F)(F)CC4)C3=O)c2c1]

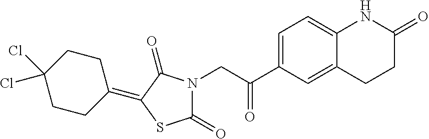 [SMILES: O=C1CCc2cc(C(=O)CN3C(=O)SC(=C4CCC(Cl)(Cl)CC4)C3=O)ccc2N1]

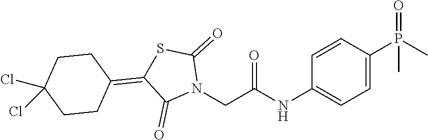 [SMILES: CP(C)(=O)c1ccc(NC(=O)CN2C(=O)SC(=C3CCC(Cl)(Cl)CC3)C2=O)cc1]

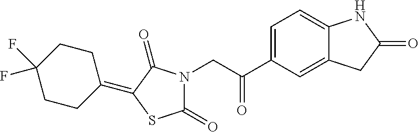 [SMILES: O=C1Cc2cc(C(=O)CN3C(=O)SC(=C4CCC(F)(F)CC4)C3=O)ccc2N1]

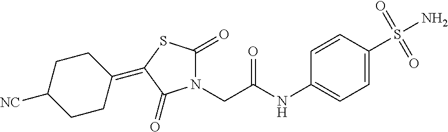 [SMILES: N#CC1CCC(=C2SC(=O)N(CC(=O)Nc3ccc(S(N)(=O)=O)cc3)C2=O)CC1]

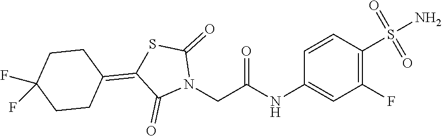 [SMILES: NS(=O)(=O)c1ccc(NC(=O)CN2C(=O)SC(=C3CCC(F)(F)CC3)C2=O)cc1F]

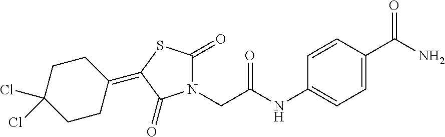 [SMILES: NC(=O)c1ccc(NC(=O)CN2C(=O)SC(=C3CCC(Cl)(Cl)CC3)C2=O)cc1]

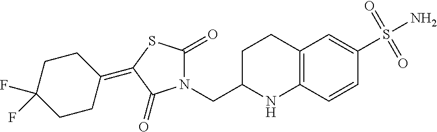 [SMILES: NS(=O)(=O)c1ccc2c(c1)CCC(CN1C(=O)SC(=C3CCC(F)(F)CC3)C1=O)N2]

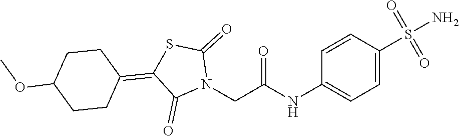 [SMILES: COC1CCC(=C2SC(=O)N(CC(=O)Nc3ccc(S(N)(=O)=O)cc3)C2=O)CC1]

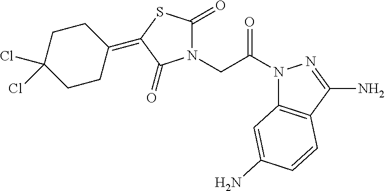 [SMILES: Nc1ccc2c(N)nn(C(=O)CN3C(=O)SC(=C4CCC(Cl)(Cl)CC4)C3=O)c2c1]

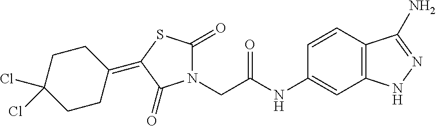 [SMILES: Nc1n[nH]c2cc(NC(=O)CN3C(=O)SC(=C4CCC(Cl)(Cl)CC4)C3=O)ccc12]

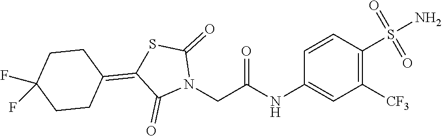 [SMILES: NS(=O)(=O)c1ccc(NC(=O)CN2C(=O)SC(=C3CCC(F)(F)CC3)C2=O)cc1C(F)(F)F]

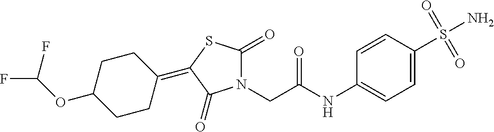 [SMILES: NS(=O)(=O)c1ccc(NC(=O)CN2C(=O)SC(=C3CCC(OC(F)F)CC3)C2=O)cc1]

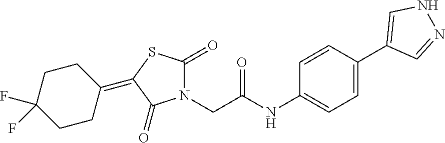 [SMILES: O=C(CN1C(=O)SC(=C2CCC(F)(F)CC2)C1=O)Nc1ccc(-c2cn[nH]c2)cc1]

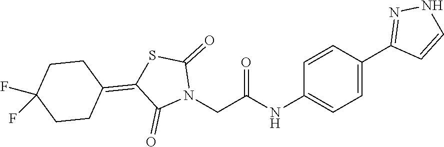 [SMILES: O=C(CN1C(=O)SC(=C2CCC(F)(F)CC2)C1=O)Nc1ccc(-c2cc[nH]n2)cc1]

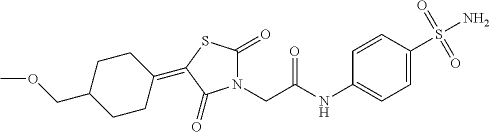 [SMILES: COCC1CCC(=C2SC(=O)N(CC(=O)Nc3ccc(S(N)(=O)=O)cc3)C2=O)CC1]

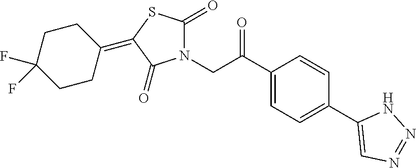 [SMILES: O=C(CN1C(=O)SC(=C2CCC(F)(F)CC2)C1=O)c1ccc(-c2cnn[nH]2)cc1]

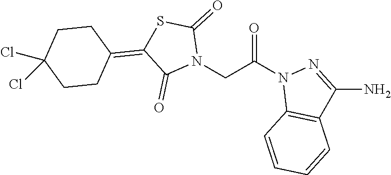 [SMILES: Nc1nn(C(=O)CN2C(=O)SC(=C3CCC(Cl)(Cl)CC3)C2=O)c2ccccc12]

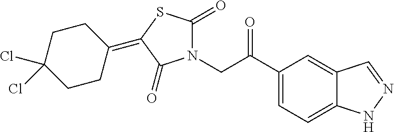 [SMILES: O=C(CN1C(=O)SC(=C2CCC(Cl)(Cl)CC2)C1=O)c1ccc2[nH]ncc2c1]

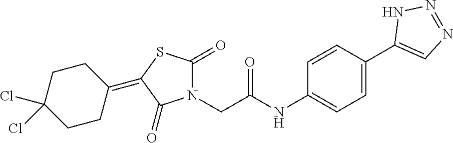 [SMILES: O=C(CN1C(=O)SC(=C2CCC(Cl)(Cl)CC2)C1=O)Nc1ccc(-c2cnn[nH]2)cc1]

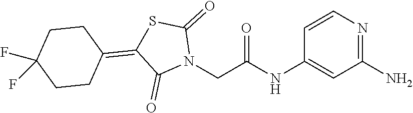 [SMILES: Nc1cc(NC(=O)CN2C(=O)SC(=C3CCC(F)(F)CC3)C2=O)ccn1]

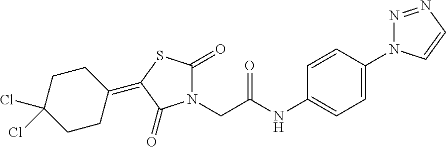 [SMILES: O=C(CN1C(=O)SC(=C2CCC(Cl)(Cl)CC2)C1=O)Nc1ccc(-n2ccnn2)cc1]

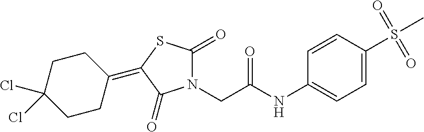 [SMILES: CS(=O)(=O)c1ccc(NC(=O)CN2C(=O)SC(=C3CCC(Cl)(Cl)CC3)C2=O)cc1]

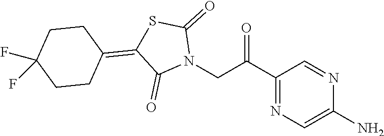 [SMILES: Nc1cnc(C(=O)CN2C(=O)SC(=C3CCC(F)(F)CC3)C2=O)cn1]

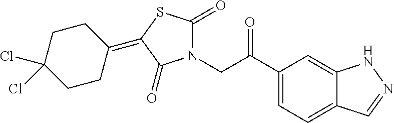 [SMILES: O=C(CN1C(=O)SC(=C2CCC(Cl)(Cl)CC2)C1=O)c1ccc2cn[nH]c2c1]

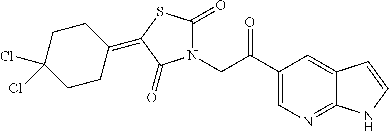 [SMILES: O=C(CN1C(=O)SC(=C2CCC(Cl)(Cl)CC2)C1=O)c1cnc2[nH]ccc2c1]

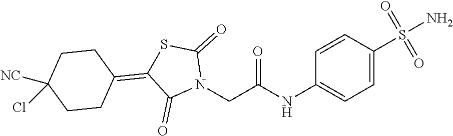 [SMILES: N#CC1(Cl)CCC(=C2SC(=O)N(CC(=O)Nc3ccc(S(N)(=O)=O)cc3)C2=O)CC1]

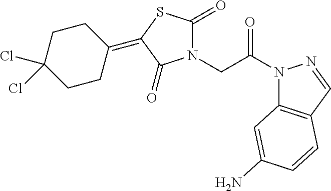 [SMILES: Nc1ccc2cnn(C(=O)CN3C(=O)SC(=C4CCC(Cl)(Cl)CC4)C3=O)c2c1]

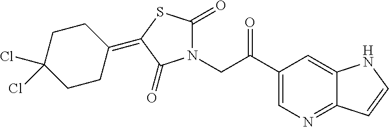 [SMILES: O=C(CN1C(=O)SC(=C2CCC(Cl)(Cl)CC2)C1=O)c1cnc2cc[nH]c2c1]

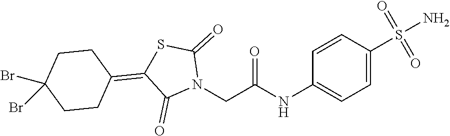 [SMILES: NS(=O)(=O)c1ccc(NC(=O)CN2C(=O)SC(=C3CCC(Br)(Br)CC3)C2=O)cc1]

TABLE 23-2

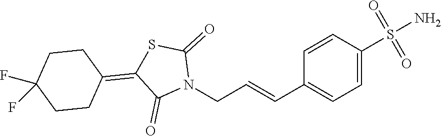 [SMILES: NS(=O)(=O)c1ccc(/C=C/CN2C(=O)SC(=C3CCC(F)(F)CC3)C2=O)cc1]

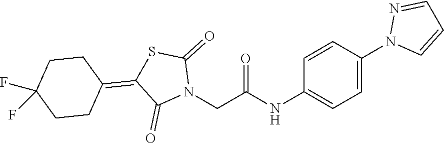 [SMILES: O=C(CN1C(=O)SC(=C2CCC(F)(F)CC2)C1=O)Nc1ccc(-n2cccn2)cc1]

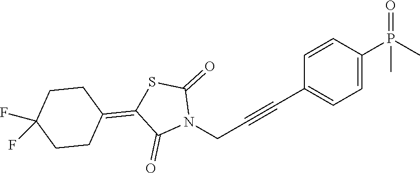 [SMILES: CP(C)(=O)c1ccc(C#CCN2C(=O)SC(=C3CCC(F)(F)CC3)C2=O)cc1]

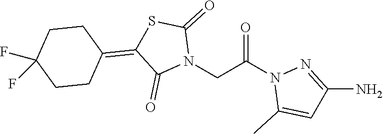 [SMILES: Cc1cc(N)nn1C(=O)CN1C(=O)SC(=C2CCC(F)(F)CC2)C1=O]

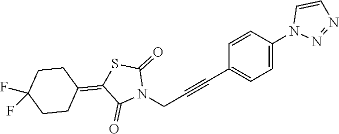 [SMILES: O=C1SC(=C2CCC(F)(F)CC2)C(=O)N1CC#Cc1ccc(-n2ccnn2)cc1]

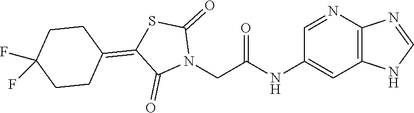 [SMILES: O=C(CN1C(=O)SC(=C2CCC(F)(F)CC2)C1=O)Nc1cnc2nc[nH]c2c1]

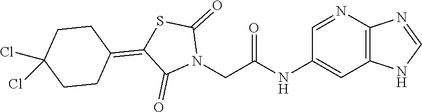 [SMILES: O=C(CN1C(=O)SC(=C2CCC(Cl)(Cl)CC2)C1=O)Nc1cnc2nc[nH]c2c1]

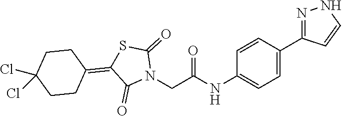 [SMILES: O=C(CN1C(=O)SC(=C2CCC(Cl)(Cl)CC2)C1=O)Nc1ccc(-c2cc[nH]n2)cc1]

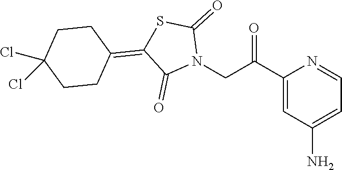 [SMILES: Nc1ccnc(C(=O)CN2C(=O)SC(=C3CCC(Cl)(Cl)CC3)C2=O)c1]

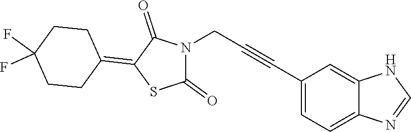 [SMILES: O=C1SC(=C2CCC(F)(F)CC2)C(=O)N1CC#Cc1ccc2nc[nH]c2c1]

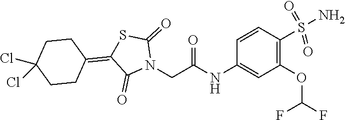 [SMILES: NS(=O)(=O)c1ccc(NC(=O)CN2C(=O)SC(=C3CCC(Cl)(Cl)CC3)C2=O)cc1OC(F)F]

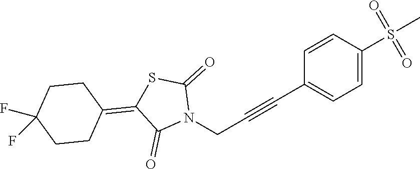 [SMILES: CS(=O)(=O)c1ccc(C#CCN2C(=O)SC(=C3CCC(F)(F)CC3)C2=O)cc1]

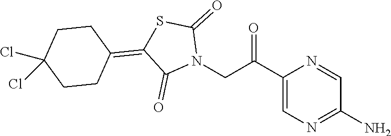 [SMILES: Nc1cnc(C(=O)CN2C(=O)SC(=C3CCC(Cl)(Cl)CC3)C2=O)cn1]

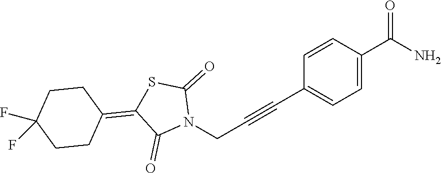 [SMILES: NC(=O)c1ccc(C#CCN2C(=O)SC(=C3CCC(F)(F)CC3)C2=O)cc1]

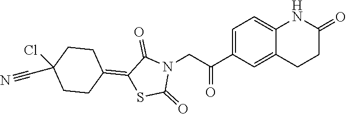 [SMILES: N#CC1(Cl)CCC(=C2SC(=O)N(CC(=O)c3ccc4c(c3)CCC(=O)N4)C2=O)CC1]

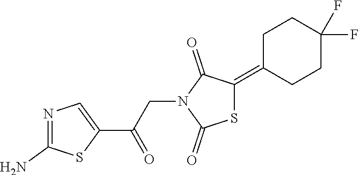 [SMILES: Nc1ncc(C(=O)CN2C(=O)SC(=C3CCC(F)(F)CC3)C2=O)s1]

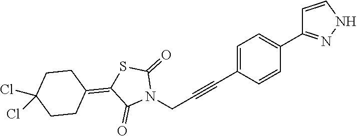 [SMILES: O=C1SC(=C2CCC(Cl)(Cl)CC2)C(=O)N1CC#Cc1ccc(-c2cc[nH]n2)cc1]

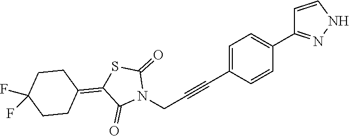 [SMILES: O=C1SC(=C2CCC(F)(F)CC2)C(=O)N1CC#Cc1ccc(-c2cc[nH]n2)cc1]

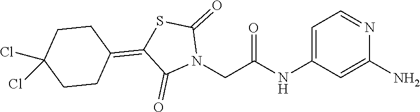 [SMILES: Nc1cc(NC(=O)CN2C(=O)SC(=C3CCC(Cl)(Cl)CC3)C2=O)ccn1]

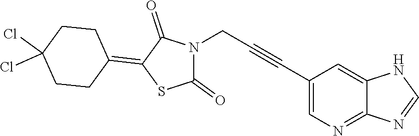 [SMILES: O=C1SC(=C2CCC(Cl)(Cl)CC2)C(=O)N1CC#Cc1cnc2nc[nH]c2c1]

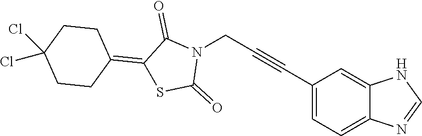 [SMILES: O=C1SC(=C2CCC(Cl)(Cl)CC2)C(=O)N1CC#Cc1ccc2nc[nH]c2c1]

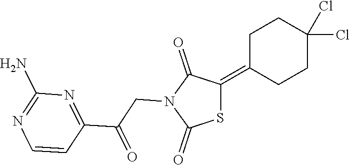 [SMILES: Nc1nccc(C(=O)CN2C(=O)SC(=C3CCC(Cl)(Cl)CC3)C2=O)n1]

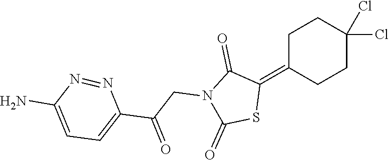 [SMILES: Nc1ccc(C(=O)CN2C(=O)SC(=C3CCC(Cl)(Cl)CC3)C2=O)nn1]

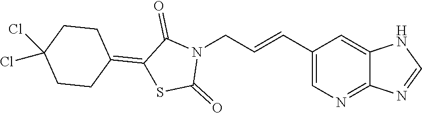 [SMILES: O=C1SC(=C2CCC(Cl)(Cl)CC2)C(=O)N1C/C=C/c1cnc2nc[nH]c2c1]

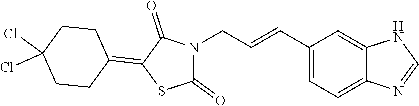 [SMILES: O=C1SC(=C2CCC(Cl)(Cl)CC2)C(=O)N1C/C=C/c1ccc2nc[nH]c2c1]

or a pharmaceutically acceptable salt thereof.

### [Item 47]

The pharmaceutical composition of item 42, wherein the low molecular weight compound is a compound represented by formula (3)

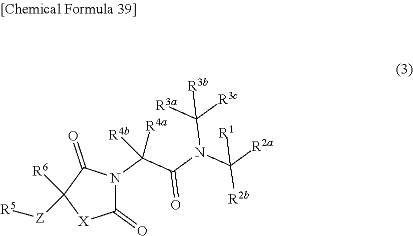

wherein
X is —NH— or O—;
Z is a direct bond or —C(R^{7a})(R^{7b})—;
R¹ is carbocyclyl or heterocyclyl;
R^{2a} and R^{2b} are each independently a hydrogen atom, a deuterium atom, C₁₋₆ alkyl, C₁₋₆ alkenyl, or C₁₋₆ alkynyl;
R^{3a} is carbocyclyl or heterocyclyl, and R^{3b} is C₁₋₆ alkyl, C₁₋₆ alkenyl, C₁₋₆ alkynyl, or carbocyclyl, or R^{3a} and R^{3b} are each independently C₁₋₆ alkyl, C₁₋₆ alkenyl, or C₁₋₆ alkynyl, wherein R^{3a} and R^{3b}, together with the carbon atom to which they are attached, may form carbocyclyl or heterocyclyl;
R^{3c} is a hydrogen atom or a deuterium atom;
R^{4a} and R^{4b} are each independently a hydrogen atom, a deuterium atom, C₁₋₆ alkyl, C₁₋₆ alkenyl, or C₁₋₆ alkynyl;
R⁵ is carbocyclyl or heterocyclyl;
R⁶ is, when Z is a direct bond, a hydrogen atom or a deuterium atom; or is, when Z is —C(R^{7a})(R^{7b})—, a hydrogen atom, a deuterium atom, C₁₋₆ alkyl, C₁₋₆ alkenyl, or C₁₋₆ alkynyl;
R^{7a} and R^{7b} are each independently a hydrogen atom, a deuterium atom, C₁₋₆ alkyl, C₁₋₆ alkenyl, or C₁₋₆ alkynyl, with the proviso that if Z is —CH₂—, R¹ is unsubstituted phenyl, and R⁵ is unsubstituted indolyl, each of R^{3a}, R^{3b}, and R^{3c} is not unsubstituted cyclopropyl, methyl, or a hydrogen atom,
   or a prodrug thereof or a pharmaceutically acceptable salt thereof.

### [Item 48]

The pharmaceutical composition of item 42, wherein the low molecular weight compound is a compound represented by the following (Table 3)

TABLE 24-1

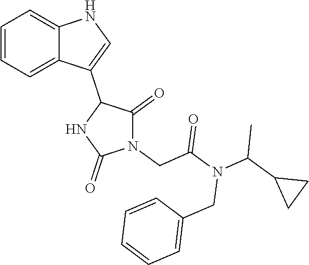 [SMILES: CC(C1CC1)N(Cc1ccccc1)C(=O)CN1C(=O)NC(c2c[nH]c3ccccc23)C1=O]

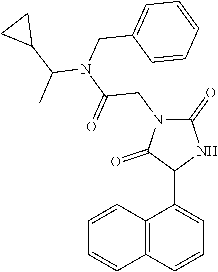 [SMILES: CC(C1CC1)N(Cc1ccccc1)C(=O)CN1C(=O)NC(c2cccc3ccccc23)C1=O]

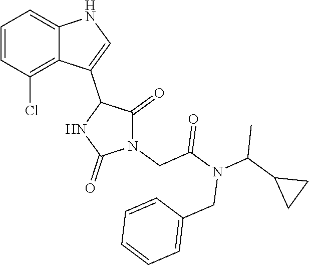 [SMILES: CC(C1CC1)N(Cc1ccccc1)C(=O)CN1C(=O)NC(c2c[nH]c3cccc(Cl)c23)C1=O]

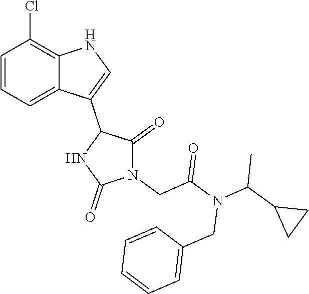 [SMILES: CC(C1CC1)N(Cc1ccccc1)C(=O)CN1C(=O)NC(c2c[nH]c3c(Cl)cccc23)C1=O]

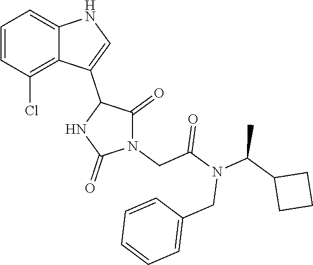 [SMILES: C[C@@H](C1CCC1)N(Cc1ccccc1)C(=O)CN1C(=O)NC(c2c[nH]c3cccc(Cl)c23)C1=O]

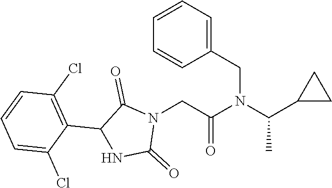 [SMILES: C[C@@H](C1CC1)N(Cc1ccccc1)C(=O)CN1C(=O)NC(c2c(Cl)cccc2Cl)C1=O]

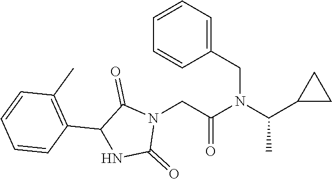 [SMILES: Cc1ccccc1C1NC(=O)N(CC(=O)N(Cc2ccccc2)[C@@H](C)C2CC2)C1=O]

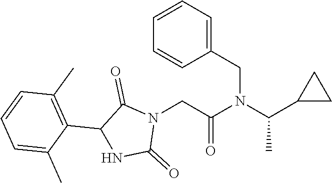 [SMILES: Cc1cccc(C)c1C1NC(=O)N(CC(=O)N(Cc2ccccc2)[C@@H](C)C2CC2)C1=O]

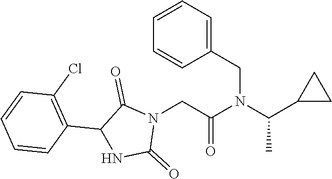 [SMILES: C[C@@H](C1CC1)N(Cc1ccccc1)C(=O)CN1C(=O)NC(c2ccccc2Cl)C1=O]

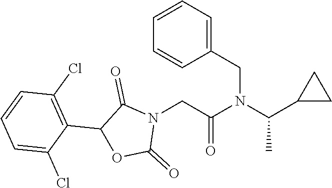 [SMILES: C[C@@H](C1CC1)N(Cc1ccccc1)C(=O)CN1C(=O)OC(c2c(Cl)cccc2Cl)C1=O]

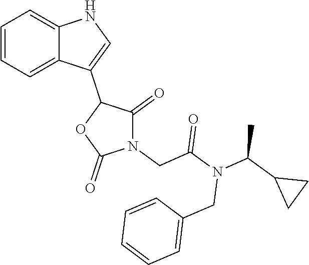 [SMILES: C[C@@H](C1CC1)N(Cc1ccccc1)C(=O)CN1C(=O)OC(c2c[nH]c3ccccc23)C1=O]

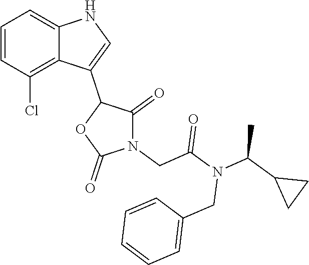 [SMILES: C[C@@H](C1CC1)N(Cc1ccccc1)C(=O)CN1C(=O)OC(c2c[nH]c3cccc(Cl)c23)C1=O]

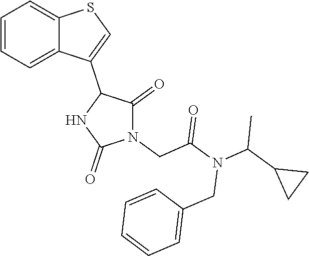 [SMILES: CC(C1CC1)N(Cc1ccccc1)C(=O)CN1C(=O)NC(c2csc3ccccc23)C1=O]

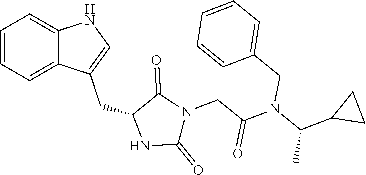 [SMILES: C[C@@H](C1CC1)N(Cc1ccccc1)C(=O)CN1C(=O)N[C@H](Cc2c[nH]c3ccccc23)C1=O]

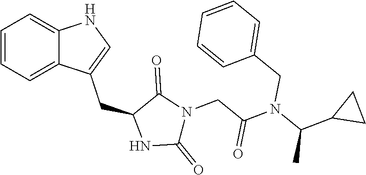 [SMILES: C[C@H](C1CC1)N(Cc1ccccc1)C(=O)CN1C(=O)N[C@@H](Cc2c[nH]c3ccccc23)C1=O]

TABLE 24-2

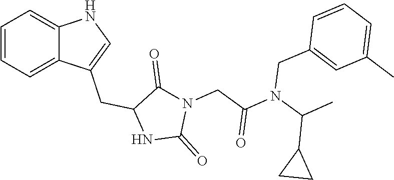 [SMILES: Cc1cccc(CN(C(=O)CN2C(=O)NC(Cc3c[nH]c4ccccc34)C2=O)C(C)C2CC2)c1]

or a pharmaceutically acceptable salt thereof.

### [Item 49]

The pharmaceutical composition of item 42, wherein the low molecular weight compound is a compound represented by formula (4)

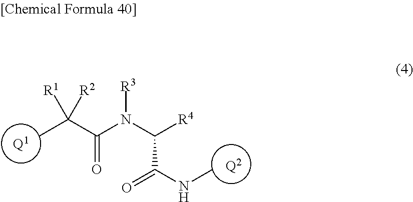

wherein
ring Q¹ represents a phenyl group optionally having 1 to 3 substituents independently selected from group A described below, or a 5- or 6-membered aromatic heterocyclic group having 1 to 3 nitrogen atoms having 1 to 3 substituents independently selected from group A described below within a ring,
ring Q² represents a phenyl group optionally having 1 to 3 substituents independently selected from group B described below, a naphthyl group optionally having 1 to 3 substituents independently selected from group B described below, a 5- or 6-membered aromatic heterocyclic group having 1 to 3 nitrogen atoms optionally having 1 to 3 substituents independently selected from group B described below within a ring, or a 8- to 10-membered bicyclic aromatic heterocyclic group optionally having 1 to 4 heteroatoms independently selected from the group consisting of a nitrogen atom, an oxygen atom, and a sulfur atom optionally having 1 to 3 substituents independently selected from group B described below within a ring,
R¹ and R² each independently represents a C₁₋₆ alkyl group or a C₁₋₆ alkoxy group, or
R¹ and R², together with the carbon atom to which R¹ and R² are attached, are a 3- to 7-membered cycloalkyl ring optionally having 1 to 3 substituents independently selected from group C described below, a tetrahydropyran ring optionally having 1 to 3 substituents independently selected from group C described below, or a dioxane ring optionally having 1 to 3 substituents independently selected from group C described below,
R³ represents a hydrogen atom, a C₁₋₆ alkyl group, or a hydroxy C₂₋₆ alkyl group,
R⁴ represents a hydrogen atom, a C₁₋₆ alkyl group, a hydroxy C₁₋₆ alkyl group, or a C₁₋₆ alkylsulfonyl C₁₋₆ alkyl group, or
R³ and R⁴, together with the nitrogen atom to which R³ is attached and the carbon atom to which R⁴ is attached, may form an azetidine ring optionally having 1 to 3 substituents independently selected from group D described below, a pyrrolidine ring optionally having 1 to 3 substituents independently selected from group D described below, a hexamethyleneimine ring optionally having 1 to 3 substituents independently selected from group D described below, a thiazolidine ring optionally having 1 to 3 substituents independently selected from group D described below, a 1-oxothiazolidine ring optionally having 1 to 3 substituents independently selected from group D described below, a 1,1′-dioxothiazolidine ring optionally having 1 to 3 substituents independently selected from group D described below, or a 4-oxopyrrolidine ring optionally having 1 to 3 substituents independently selected from group D described below,
wherein, group A is a halogen atom, a hydroxy group, a carboxy group, a C₁₋₆ alkyl group, a hydroxy C₁₋₆ alkyl group, a C₁₋₆ alkoxy group, a halogeno C₁₋₆ alkoxy group, a C₁₋₆ alkoxycarbonyl group, a C₂₋₇ alkanoyl group, a halogeno C₂₋₇ alkanoyl group, a C₂₋₇ alkanoylamino group, a C₁₋₆ alkylsulfonyl group, a C₁₋₆ alkylsulfonylamino group, a C₃₋₇ cycloalkylsulfonylamino group, a phenyl group, a phenylsulfonylamino group, a carbamoyl group, a C₁₋₆ alkylcarbamoyl group, a di-C₁₋₆ alkylcarbamoyl group, a benzyloxycarbonyl group, a C₃₋₇ cycloalkylsulfonylcarbamoyl group, a halogeno C₁₋₆ alkylsulfonyloxy group, and a phenylsulfonyl group,
group B is a halogen atom, a cyano group, an amino group, a C₁₋₆ alkyl group, a C₁₋₆ alkoxy group, a hydroxy C₁₋₆ alkyl group, a C₁₋₆ alkylamino group, a C₁₋₆ alkylamino C₁₋₆ alkyl group, a morpholinyl C₁₋₆ alkyloxy group, a phenyl group, a benzyloxy group, a C₁₋₆ alkoxy C₁₋₆ alkyl group, a hydroxy group, a halogeno C₁₋₆ alkyl group, a C₁₋₆ alkoxycarbonyl group, a C₂₋₇ alkanoylamino group, a halogeno C₁₋₆ alkoxy group, a C₁₋₆ alkoxy C₁₋₆ alkoxy group, a C₁₋₆ alkylsulfonylamino group, a morpholinyl C₁₋₆ alkyl group, and a C₁₋₆ alkylsulfonyl group,
group C is a halogen atom, a C₁₋₆ alkyl group, and a C₁₋₆ alkoxy group, and
group D is a halogen atom, a hydroxy group, a C₁₋₆ alkyl group, a C₁₋₆ alkoxy group, a C₁₋₆ alkoxy C₁₋₆ alkoxy group, a C₂₋₆ alkynyl group, a C₂₋₇ alkanoylamino group, an amino group, and a di-C₁₋₆ alkylamino group,
   or a prodrug thereof or a pharmaceutically acceptable salt thereof.

### [Item 50]

The pharmaceutical composition of item 42, wherein the low molecular weight compound is a compound represented by the following (Table 4)

TABLE 25

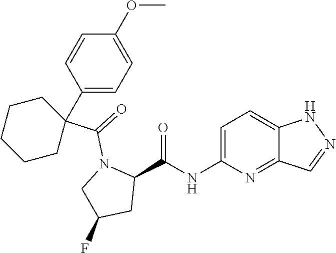 [SMILES: COc1ccc(C2(C(=O)N3C[C@H](F)C[C@@H]3C(=O)Nc3ccc4[nH]ncc4n3)CCCCC2)cc1]

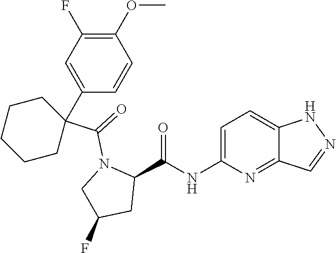 [SMILES: COc1ccc(C2(C(=O)N3C[C@H](F)C[C@@H]3C(=O)Nc3ccc4[nH]ncc4n3)CCCCC2)cc1F]

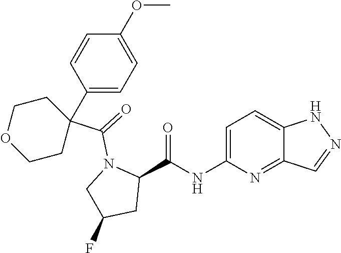 [SMILES: COc1ccc(C2(C(=O)N3C[C@H](F)C[C@@H]3C(=O)Nc3ccc4[nH]ncc4n3)CCOCC2)cc1]

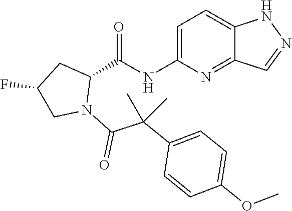 [SMILES: COc1ccc(C(C)(C)C(=O)N2C[C@H](F)C[C@@H]2C(=O)Nc2ccc3[nH]ncc3n2)cc1]

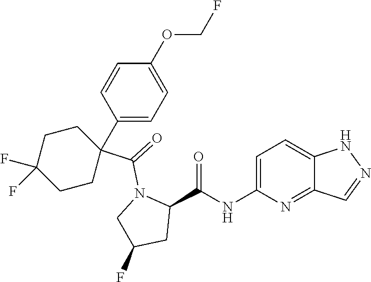 [SMILES: O=C(Nc1ccc2[nH]ncc2n1)[C@H]1C[C@@H](F)CN1C(=O)C1(c2ccc(OCF)cc2)CCC(F)(F)CC1]

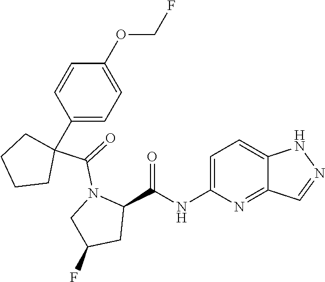 [SMILES: O=C(Nc1ccc2[nH]ncc2n1)[C@H]1C[C@@H](F)CN1C(=O)C1(c2ccc(OCF)cc2)CCCC1]

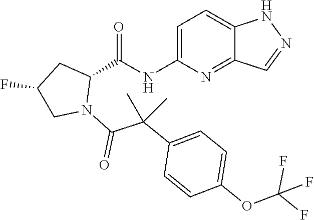 [SMILES: CC(C)(C(=O)N1C[C@H](F)C[C@@H]1C(=O)Nc1ccc2[nH]ncc2n1)c1ccc(OC(F)(F)F)cc1]

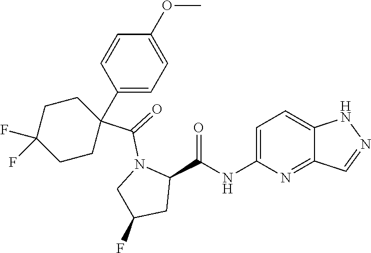 [SMILES: COc1ccc(C2(C(=O)N3C[C@H](F)C[C@@H]3C(=O)Nc3ccc4[nH]ncc4n3)CCC(F)(F)CC2)cc1]

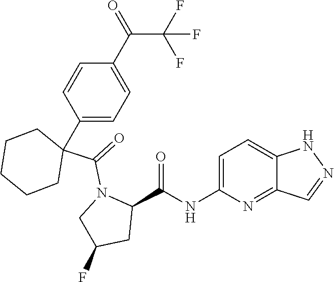 [SMILES: O=C(Nc1ccc2[nH]ncc2n1)[C@H]1C[C@@H](F)CN1C(=O)C1(c2ccc(C(=O)C(F)(F)F)cc2)CCCCC1]

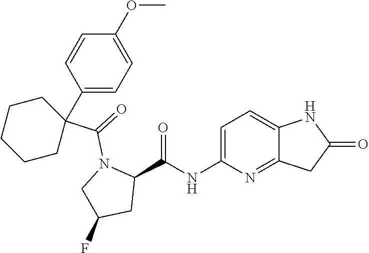 [SMILES: COc1ccc(C2(C(=O)N3C[C@H](F)C[C@@H]3C(=O)Nc3ccc4c(n3)CC(=O)N4)CCCCC2)cc1]

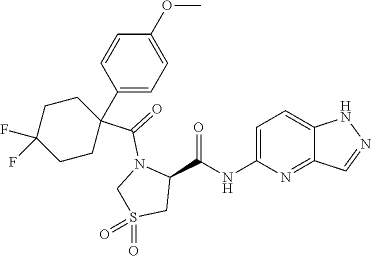 [SMILES: COc1ccc(C2(C(=O)N3CS(=O)(=O)C[C@@H]3C(=O)Nc3ccc4[nH]ncc4n3)CCC(F)(F)CC2)cc1]

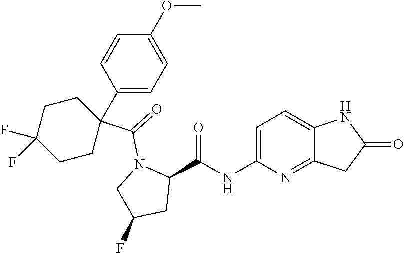 [SMILES: COc1ccc(C2(C(=O)N3C[C@H](F)C[C@@H]3C(=O)Nc3ccc4c(n3)CC(=O)N4)CCC(F)(F)CC2)cc1]

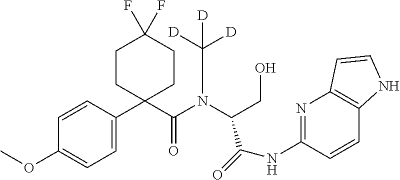 [SMILES: [2H]C([2H])([2H])N(C(=O)C1(c2ccc(OC)cc2)CCC(F)(F)CC1)[C@H](CO)C(=O)Nc1ccc2[nH]ccc2n1]

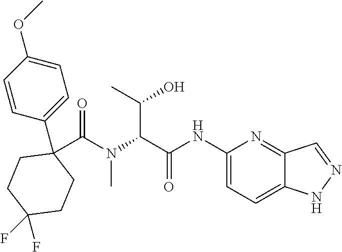 [SMILES: COc1ccc(C2(C(=O)N(C)[C@@H](C(=O)Nc3ccc4[nH]ncc4n3)[C@H](C)O)CCC(F)(F)CC2)cc1]

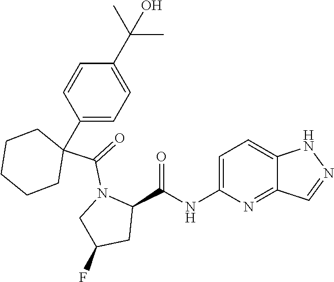 [SMILES: CC(C)(O)c1ccc(C2(C(=O)N3C[C@H](F)C[C@@H]3C(=O)Nc3ccc4[nH]ncc4n3)CCCCC2)cc1]

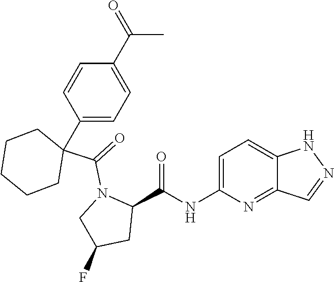 [SMILES: CC(=O)c1ccc(C2(C(=O)N3C[C@H](F)C[C@@H]3C(=O)Nc3ccc4[nH]ncc4n3)CCCCC2)cc1]

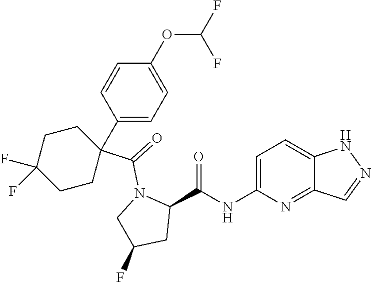 [SMILES: O=C(Nc1ccc2[nH]ncc2n1)[C@H]1C[C@@H](F)CN1C(=O)C1(c2ccc(OC(F)F)cc2)CCC(F)(F)CC1]

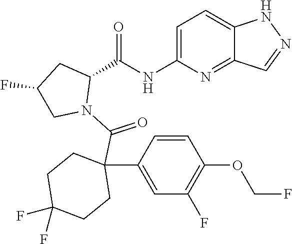 [SMILES: O=C(Nc1ccc2[nH]ncc2n1)[C@H]1C[C@@H](F)CN1C(=O)C1(c2ccc(OCF)c(F)c2)CCC(F)(F)CC1]

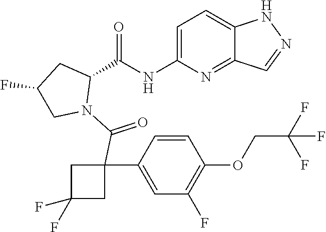 [SMILES: O=C(Nc1ccc2[nH]ncc2n1)[C@H]1C[C@@H](F)CN1C(=O)C1(c2ccc(OCC(F)(F)F)c(F)c2)CC(F)(F)C1]

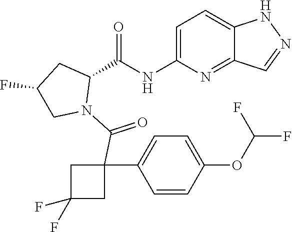 [SMILES: O=C(Nc1ccc2[nH]ncc2n1)[C@H]1C[C@@H](F)CN1C(=O)C1(c2ccc(OC(F)F)cc2)CC(F)(F)C1]

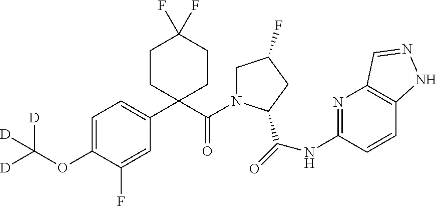 [SMILES: [2H]C([2H])([2H])Oc1ccc(C2(C(=O)N3C[C@H](F)C[C@@H]3C(=O)Nc3ccc4[nH]ncc4n3)CCC(F)(F)CC2)cc1F]

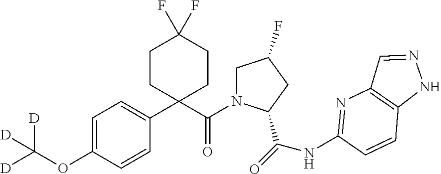 [SMILES: [2H]C([2H])([2H])Oc1ccc(C2(C(=O)N3C[C@H](F)C[C@@H]3C(=O)Nc3ccc4[nH]ncc4n3)CCC(F)(F)CC2)cc1]

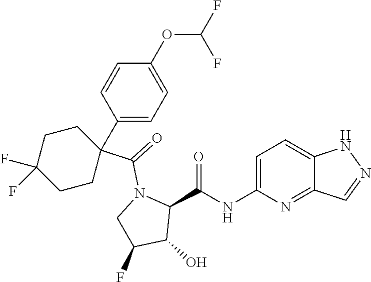 [SMILES: O=C(Nc1ccc2[nH]ncc2n1)[C@H]1[C@H](O)[C@@H](F)CN1C(=O)C1(c2ccc(OC(F)F)cc2)CCC(F)(F)CC1]

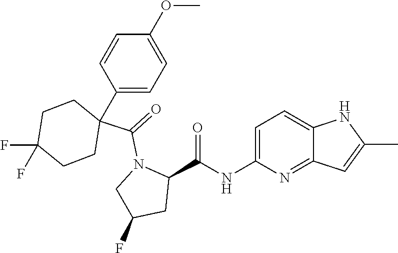 [SMILES: COc1ccc(C2(C(=O)N3C[C@H](F)C[C@@H]3C(=O)Nc3ccc4[nH]c(C)cc4n3)CCC(F)(F)CC2)cc1]

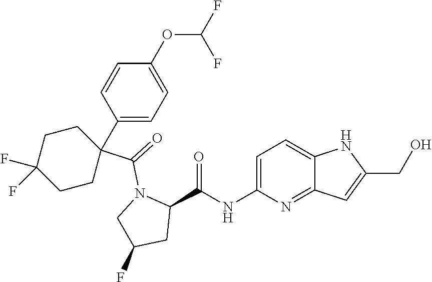 [SMILES: O=C(Nc1ccc2[nH]c(CO)cc2n1)[C@H]1C[C@@H](F)CN1C(=O)C1(c2ccc(OC(F)F)cc2)CCC(F)(F)CC1]

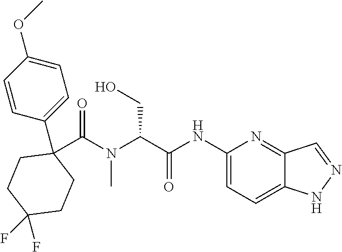 [SMILES: COc1ccc(C2(C(=O)N(C)[C@H](CO)C(=O)Nc3ccc4[nH]ncc4n3)CCC(F)(F)CC2)cc1]

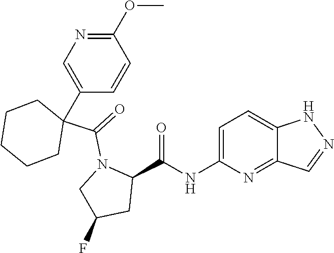 [SMILES: COc1ccc(C2(C(=O)N3C[C@H](F)C[C@@H]3C(=O)Nc3ccc4[nH]ncc4n3)CCCCC2)cn1]

or a pharmaceutical acceptable salt thereof.

### [Item 51]

The pharmaceutical composition of item 42, wherein the low molecular weight compound is a compound represented by formula (5)

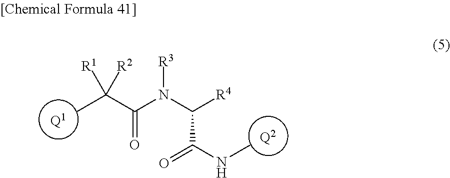

wherein
ring Q¹ represents a 3- to 7-membered cycloalkyl group optionally having 1 to 3 substituents independently selected from group A described below, a 3- to 7-membered heterocycloalkyl group having 1 to 2 heteroatoms independently selected from the group consisting of a nitrogen atom, an oxygen atom, and a sulfur atom optionally having 1 to 3 substituents independently selected from group A described below within a ring, or an 8- to 10-membered bicyclic heterocycloalkyl group having 1 to 3 heteroatoms independently selected from the group consisting of a nitrogen atom, an oxygen atom, and a sulfur atom optionally having 1 to 3 substituents independently selected from group A described below within a ring,
ring Q² represents a phenyl group optionally having 1 to 3 substituents independently selected from group B described below, a naphthyl group optionally having 1 to 3 substituents independently selected from group B described below, a 5- or 6-membered aromatic heterocyclic group having 1 to 3 nitrogen atoms optionally having 1 to 3 substituents independently selected from group B described below within a ring, or an 8- to 10-membered bicyclic aromatic heterocyclic group having 1 to 4 heteroatoms independently selected from the group consisting of a nitrogen atom, an oxygen atom, and a sulfur atom optionally having 1 to 3 substituents independently selected from group B described below within a ring,
R¹ and R² each independently represents a C₁₋₆ alkyl group or a C₁₋₆ alkoxy group, or
R¹ and R², together with the carbon atom to which R¹ and R² are attached, represent a 3- to 7-membered cycloalkyl ring optionally having 1 to 3 substituents independently selected from group C described below, a tetrahydropyran ring optionally having 1 to 3 substituents independently selected from group C described below, or a dioxane ring optionally having 1 to 3 substituents independently selected from group C described below,
R³ represents a hydrogen atom, a C₁₋₆ alkyl group, or a hydroxy C₂₋₆ alkyl group,
R⁴ represents a hydrogen atom, a C₁₋₆ alkyl group, a hydroxy C₁₋₆ alkyl group, or a C₁₋₆ alkylsulfonyl C₁₋₆ alkyl group, or
R³ and R⁴, together with the nitrogen atom to which R³ is attached and the carbon atom to which R⁴ is attached, may form an azetidine ring optionally having 1 to 3 substituents independently selected from group D described below, a pyrrolidine ring optionally having 1 to 3 substituents independently selected from group D described below, a hexamethyleneimine ring optionally having 1 to 3 substituents independently selected from group D described below, a thiazolidine ring optionally having 1 to 3 substituents independently selected from group D described below, a 1-oxothiazolidine ring optionally having 1 to 3 substituents independently selected from group D described below, a 1,1-dioxothiazolidine ring optionally having 1 to 3 substituents independently selected from group D described below, or a 4-oxopyrrolidine ring optionally having 1 to 3 substituents independently selected from group D described below,
wherein group A is a halogen atom, a hydroxy group, a carboxy group, an amino group, a C₁₋₆ alkyl group, a halogeno C₁₋₆ alkyl group, a hydroxy C₁₋₆ alkyl group, a C₁₋₆ alkoxy C₁₋₆ alkyl group, a C₁₋₆ alkoxy group, a halogeno C₁₋₆ alkoxy group, a C₁₋₆ alkoxy C₁₋₆ alkoxy group, a C₂₋₇ alkanoyl group, a hydroxy C₂₋₇ alkanoyl group, a C₂₋₇ alkanoylamino group, a C₁₋₆ alkylsulfonyl group, a C₁₋₆ alkylsulfonylamino group, a benzyl group, a benzyloxy group, and an oxo group,
group B is a halogen atom, a cyano group, an amino group, a C₁₋₆ alkyl group, a C₁₋₆ alkoxy group, a hydroxy C₁₋₆ alkyl group, a C₁₋₆ alkylamino group, a C₁₋₆ alkylamino C₁₋₆ alkyl group, a morpholinyl C₁₋₆ alkyloxy group, a phenyl group, a benzyloxy group, a C₁₋₆ alkoxy C₁₋₆ alkyl group, a hydroxy group, a halogeno C₁₋₆ alkyl group, a C₁₋₆ alkoxycarbonyl group, a C₂₋₇ alkanoylamino group, a halogeno C₁₋₆ alkoxy group, a C₁₋₆ alkoxy C₁₋₆ alkoxy group, a C₁₋₆ alkylsulfonylamino group, a morpholinyl C₁₋₆ alkyl group, and a C₁₋₆ alkylsulfonyl group,
group C is a halogen atom, a C₁₋₆ alkyl group, and a C₁₋₆ alkoxy group, and
group D is a halogen atom, a hydroxy group, a C₁₋₆ alkyl group, a C₁₋₆ alkoxy group, a C₁₋₆ alkoxy C₁₋₆ alkoxy group, a C₂₋₆ alkynyl group, a C₂₋₇ alkanoylamino group, an amino group, and a di-C₁₋₆ alkylamino group,
   or a prodrug thereof or a pharmaceutically acceptable salt thereof.

### [Item 52]

The pharmaceutical composition of item 42, wherein the low molecular weight compound is a compound represented by the following (Table 5)

TABLE 26

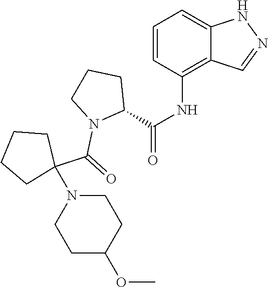 [SMILES: COC1CCN(C2(C(=O)N3CCC[C@@H]3C(=O)Nc3cccc4[nH]ncc34)CCCC2)CC1]

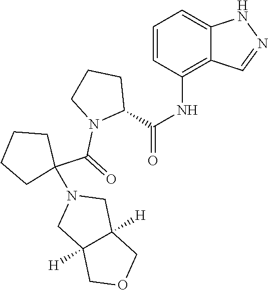 [SMILES: O=C(Nc1cccc2[nH]ncc12)[C@H]1CCCN1C(=O)C1(N2C[C@H]3COC[C@H]3C2)CCCC1]

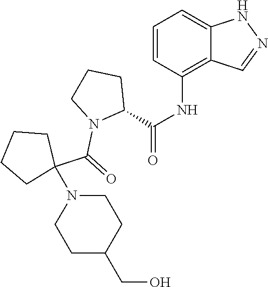 [SMILES: O=C(Nc1cccc2[nH]ncc12)[C@H]1CCCN1C(=O)C1(N2CCC(CO)CC2)CCCC1]

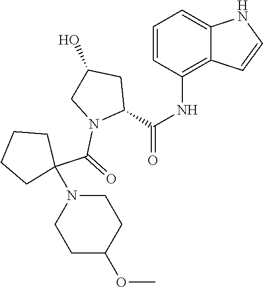 [SMILES: COC1CCN(C2(C(=O)N3C[C@H](O)C[C@@H]3C(=O)Nc3cccc4[nH]ccc34)CCCC2)CC1]

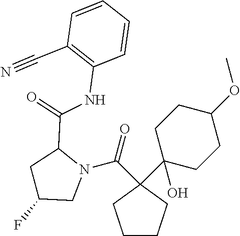 [SMILES: COC1CCC(O)(C2(C(=O)N3C[C@H](F)CC3C(=O)Nc3ccccc3C#N)CCCC2)CC1]

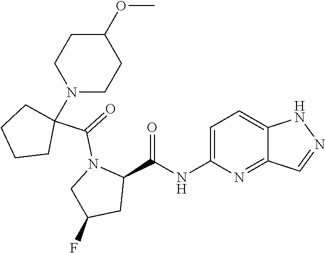 [SMILES: COC1CCN(C2(C(=O)N3C[C@H](F)C[C@@H]3C(=O)Nc3ccc4[nH]ncc4n3)CCCC2)CC1]

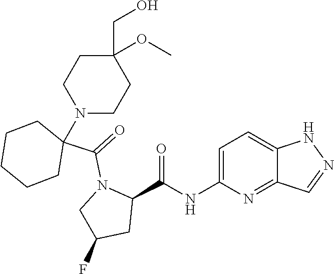 [SMILES: COC1(CO)CCN(C2(C(=O)N3C[C@H](F)C[C@@H]3C(=O)Nc3ccc4[nH]ncc4n3)CCCCC2)CC1]

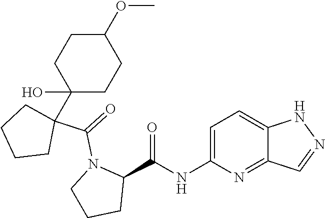 [SMILES: COC1CCC(O)(C2(C(=O)N3CCC[C@@H]3C(=O)Nc3ccc4[nH]ncc4n3)CCCC2)CC1]

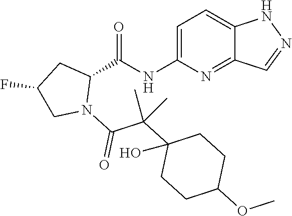 [SMILES: COC1CCC(O)(C(C)(C)C(=O)N2C[C@H](F)C[C@@H]2C(=O)Nc2ccc3[nH]ncc3n2)CC1]

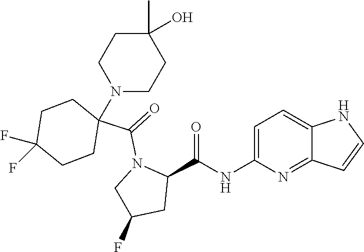 [SMILES: CC1(O)CCN(C2(C(=O)N3C[C@H](F)C[C@@H]3C(=O)Nc3ccc4[nH]ccc4n3)CCC(F)(F)CC2)CC1]

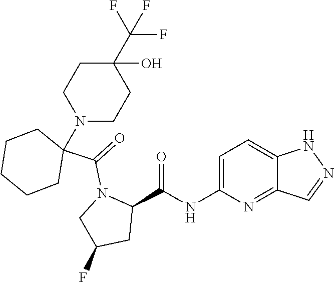 [SMILES: O=C(Nc1ccc2[nH]ncc2n1)[C@H]1C[C@@H](F)CN1C(=O)C1(N2CCC(O)(C(F)(F)F)CC2)CCCCC1]

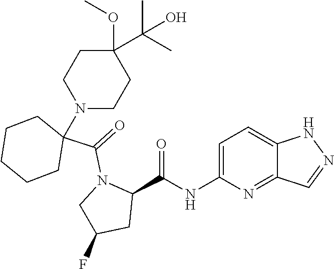 [SMILES: COC1(C(C)(C)O)CCN(C2(C(=O)N3C[C@H](F)C[C@@H]3C(=O)Nc3ccc4[nH]ncc4n3)CCCCC2)CC1]

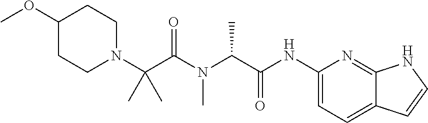 [SMILES: COC1CCN(C(C)(C)C(=O)N(C)[C@H](C)C(=O)Nc2ccc3cc[nH]c3n2)CC1]

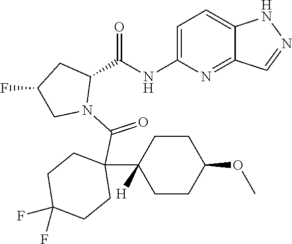 [SMILES: CO[C@H]1CC[C@H](C2(C(=O)N3C[C@H](F)C[C@@H]3C(=O)Nc3ccc4[nH]ncc4n3)CCC(F)(F)CC2)CC1]

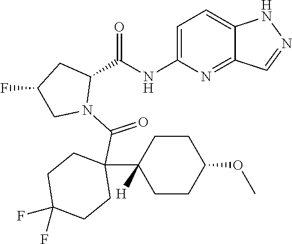 [SMILES: CO[C@H]1CC[C@@H](C2(C(=O)N3C[C@H](F)C[C@@H]3C(=O)Nc3ccc4[nH]ncc4n3)CCC(F)(F)CC2)CC1]

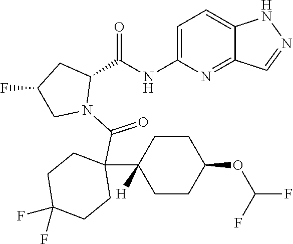 [SMILES: O=C(Nc1ccc2[nH]ncc2n1)[C@H]1C[C@@H](F)CN1C(=O)C1([C@H]2CC[C@H](OC(F)F)CC2)CCC(F)(F)CC1]

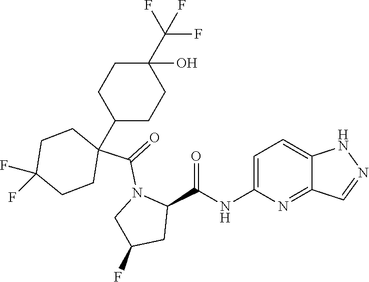 [SMILES: O=C(Nc1ccc2[nH]ncc2n1)[C@H]1C[C@@H](F)CN1C(=O)C1(C2CCC(O)(C(F)(F)F)CC2)CCC(F)(F)CC1]

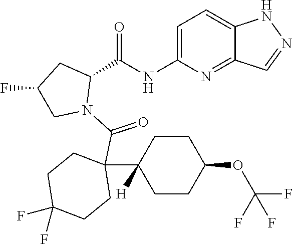 [SMILES: O=C(Nc1ccc2[nH]ncc2n1)[C@H]1C[C@@H](F)CN1C(=O)C1([C@H]2CC[C@H](OC(F)(F)F)CC2)CCC(F)(F)CC1]

or a pharmaceutically acceptable salt thereof.

### [Item 53]

The pharmaceutical composition of item 42, wherein the low molecular weight compound is a compound represented by formula (6)

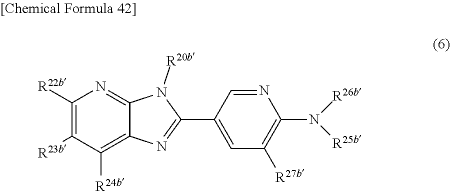

wherein
R^{20b′} is C₁₋₂ alkyl (wherein the alkyl group is substituted with phenyl substituted with pyrimidinyl, pyrazolyl, pyrazolyl substituted with C₁₋₃ alkyl, pyrazinyl, pyrazinyl substituted with C₁₋₃ alkyl, piperazinyl, piperazinyl substituted with oxo, piperazinyl substituted with C₁₋₃ alkyl, oxazolyl, oxazolyl substituted with C₁₋₃ alkyl, imidazolyl, imidazolyl substituted with C₁₋₃ alkyl, morpholinyl, morpholinyl substituted with 1 to 2 C₁₋₃ alkyl, morpholinyl substituted with oxo, dioxanyl, dioxanyl substituted with C₁₋₃ alkyl, 4,5,6,7-tetrahydropyrazolo[1,5-a]pyrazine, triazolyl, triazolyl substituted with C₁₋₃ alkyl, thiazolyl, thiazolyl substituted with C₁₋₃ alkyl, cyclopentyloxy, C₁₋₆ alkoxy, C₁₋₆ alkoxy substituted with 1 to 6 fluoro, C₁₋₆ alkoxy substituted with hydroxy, tetrahydrofuran, pyridyl, pyridyl substituted with bromo, or pyridyl substituted with pyrimidinyl);
R^{22b′}, R^{23b′}, and R^{24b′} are each independently selected from a hydrogen atom, fluoro, chloro, bromo, —OH, boronic acid, 1,3,6,2-dioxazaborocane-4,8-dione, —CN, —C(O)NHCH₃, —C(O)NHCH₂CH₃, —C(O)NHCH₂CF₂H, —C(O)NHCH₂CH₂OH, —C(O)NHCH₂CH₂SO₂CH₃, —C(O)NHOCH₃, —C(O)NH₂, —C(O)OCH₃, —C(O)NHCH₂ cyclopropyl, —C(O)NH cyclobutyl (wherein the group is optionally substituted with hydroxy), —CH morpholinyl, —CH₂OH, —CH₂NHCH₂CF₃, —CH₂NHCH₂CH₂SO₂CH₃, —CH₂SO₂CH₃, —CH(OH)CF₃, —CH₃, —CF₃, —OCH₃, —OCD₃, —NHC(O)CH₃, —NH₂, —NHSO₂CH₃, morpholinyl, pyrazolyl, oxazolyl, or oxazolyl substituted with 1 to 2 methyl;
R^{23b′} and R^{24′}, together with the carbon atom to which they are attached, may form oxaborolyl (wherein the group is optionally substituted with hydroxy);
R^{25b′} and R^{26b′} are each independently selected from C₁₋₃ alkyl, C₁₋₃ alkyl substituted with 1 to 3 fluoro, or cyclopropyl;
wherein R^{25b′} and R^{26b′}, together with the nitrogen atom to which they are attached, may form azetidinyl or pyrrolidinyl (wherein the group is optionally substituted with 1 to 2 C₁₋₃ alkyl, or C₁₋₃ alkyl substituted with 1 to 3 fluoro), or
one of R^{25b′} and R^{26b′} may form pyrrolidinyl or morpholinyl with R^{27b′} and any one heteroatom (wherein the group is optionally substituted with 1 to 4 C₁₋₃ alkyl);
R^{27b′} is selected from a hydrogen atom and fluoro;
wherein one of R^{25b′} and R^{26b′} may form pyrrolidinyl or morpholinyl with R^{27b′} and any one heteroatom (wherein the group is optionally substituted with 1 to 4 C₁₋₃ alkyl),
   or a prodrug thereof or a pharmaceutically acceptable salt thereof.

### [Item 54]

The pharmaceutical composition of item 42, wherein the low molecular weight compound is a compound represented by the following (Table 6)

TABLE 27

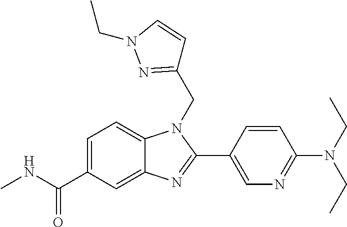 [SMILES: CCN(CC)c1ccc(-c2nc3cc(C(=O)NC)ccc3n2Cc2ccn(CC)n2)cn1]

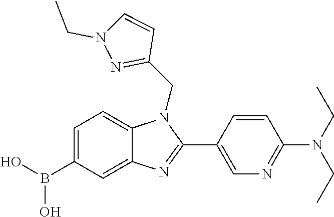 [SMILES: CCN(CC)c1ccc(-c2nc3cc(B(O)O)ccc3n2Cc2ccn(CC)n2)cn1]

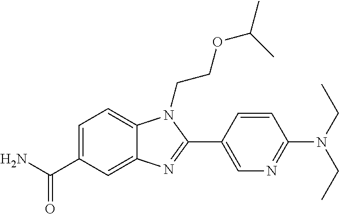 [SMILES: CCN(CC)c1ccc(-c2nc3cc(C(N)=O)ccc3n2CCOC(C)C)cn1]

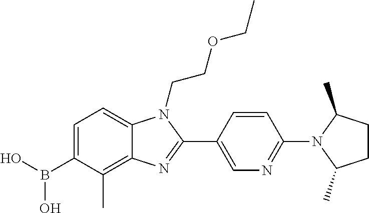 [SMILES: CCOCCn1c(-c2ccc(N3[C@@H](C)CC[C@@H]3C)nc2)nc2c(C)c(B(O)O)ccc21]

or a pharmaceutically acceptable salt thereof.

### [Item 55]

The pharmaceutical composition of item 42, wherein the low molecular weight compound is a compound represented by formula (7)

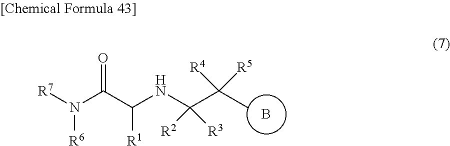

wherein
ring B is aryl, heterocyclyl, or heteroaryl (wherein the ring is each optionally substituted with 1 to 4 substituents selected from R^{b});
R⁶ is a hydrogen atom or C₁₋₆ alkyl;
R⁷ is aryl or heteroaryl (wherein the group is each substituted with a substituent selected from R^{f}, and optionally substituted with 1 to 4 substituents selected from R³);
wherein R⁶ and R⁷, together with the nitrogen ring to which they are attached, may form a fused bicyclic heterocyclyl optionally substituted with 1 to 4 groups selected from R^{a};
R¹ is C₁₋₆ alkyl, C₁₋₆ haloalkyl, C₂₋₆ alkenyl, —C₁₋₆ alkyl OR^{c}, —C₁₋₆ alkyl N(R^{d})₂, —C₁₋₆ alkyl C(O)OR^{d}, —C₁₋₆ alkyl OC₁₋₆ alkyl N(R^{d})₂, —C₁₋₆ alkyl SOR^{d}, —C₁₋₆ alkyl S(O)₂R^{d}, —C₁₋₆ alkyl SON(R^{d})₂, —C₁₋₆ alkyl SO₂N(R^{d})₂, —C₁₋₆ alkylcycloalkyl, —C₁₋₆ alkylheterocyclyl, —C₁—F alkylheteroaryl, —C₁₋₆ alkylaryl, cycloalkyl, aryl, heteroaryl, or heterocyclyl (wherein each of the cycloalkyl, heterocyclyl, aryl, and heteroaryl, alone or attached to —C₁₋₆ alkylcycloalkyl, —C₁₋₆ alkylheterocyclyl, —C₁₋₆ alkylheteroaryl, or —C₁₋₆ alkylaryl, is optionally substituted with 1 to 3 groups selected from R^{c});
R², R³, R⁴, and R⁵ are each independently a hydrogen atom or C₁₋₆ alkyl (wherein the C₁₋₆ alkyl is optionally substituted with 1 to 2 substituents selected from a halogen atom, —C(O)OR^{d}, —OC₁₋₆ alkyl N(R^{d})₂, —C₁₋₆ alkyl N(R^{d})₂, —N(R^{d})₂, —NR^{d}C₁₋₆ alkyl OR^{d}, —SOR^{d}, —S(O)₂R^{d}, —SON(R^{d})₂, —SO₂N(R^{d})₂, C₃₋₁₀ cycloalkyl, C₅₋₁₀ heterocyclyl, C₅₋₁₀ heteroaryl, and C₆₋₁₀ aryl);
R^{a}, R^{b}, and R^{c} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O)OR^{d}, —C(O)N(R^{d})₂, —C(O)NR^{d}C₁₋₆ alkyl OR^{d}, —OC₁₋₆ alkyl N(R^{d})₂, —C₁₋₆ alkyl C(O)N(R^{d})₂, —C₁₋₆ alkyl N(R^{d})₂, —N(R^{d})₂, —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)₂R^{d}, —SON(R^{d})₂, —SO₂N(R^{d})₂, —SF₅, —O— cycloalkyl, —O—C₁₋₄ alkyl-aryl, —C₁₋₆ alkylcycloalkyl, —C₁₋₆ alkylaryl, —C₁₋₆ alkylheteroaryl, —C₁₋₆ alkylheterocyclyl, cycloalkyl, heterocyclyl, heteroaryl, or aryl (wherein each of the cycloalkyl, heterocyclyl, aryl, and heteroaryl, alone or attached to —O-cycloalkyl, —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 groups selected from halogen, C₁₋₆ alkyl, C₁₋₆ haloalkyl, C₁₋₆ alkoxy, C₁₋₆ haloalkoxy, —N(R^{d}), —C(O)R^{d}, and —C₁₋₆ alkyl OR^{d});
R^{d} is independently a hydrogen atom, C₁₋₆ haloalkyl, or C₁₋₆ alkyl;
R^{f} is independently cycloalkyl, heterocyclyl, heteroaryl, or aryl (wherein each of the cycloalkyl, heterocyclyl, aryl, and heteroaryl is optionally substituted with 1 to 3 substituents selected from a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O)OR^{d}, —C(O)N(R^{d})₂, —C(O)NR^{d}C₁₋₆ alkyl OR^{d}, —OC₁₋₆ alkyl N(R^{d})₂, —C₁₋₆ alkyl C(O)N(R^{d})₂, —C₁₋₆ alkyl N(R^{d})₂, —N(R^{d})₂, —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)₂R^{d}, —SON(R^{d})₂, —SO₂N(R^{d})₂, —SF₅, and —O-cycloalkyl);
wherein the compound is not N-[1,1′-biphenyl]-2-yl-2-[[2-(3,4-dimethoxyphenyl)ethyl]amino]-propanamide, 2-[(2-phenylpropyl)amino]-N-[4-(1H-1,2,4-triazol-1-yl)phenyl]-propanamide, or a salt thereof,
   or a prodrug thereof or a pharmaceutically acceptable salt thereof.

### [Item 56]

The pharmaceutical composition of item 42, wherein the low molecular weight compound is a compound represented by the following (Table 7)

TABLE 28

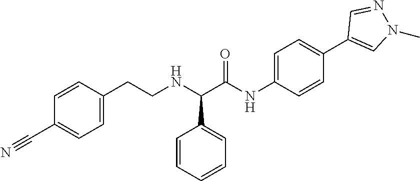 [SMILES: Cn1cc(-c2ccc(NC(=O)[C@H](NCCc3ccc(C#N)cc3)c3ccccc3)cc2)cn1]

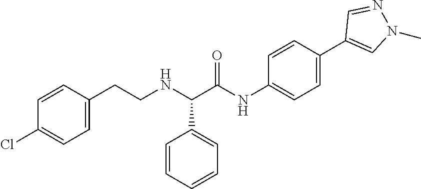 [SMILES: Cn1cc(-c2ccc(NC(=O)[C@@H](NCCc3ccc(Cl)cc3)c3ccccc3)cc2)cn1]

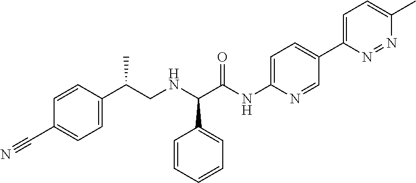 [SMILES: Cc1ccc(-c2ccc(NC(=O)[C@H](NC[C@@H](C)c3ccc(C#N)cc3)c3ccccc3)nc2)nn1]

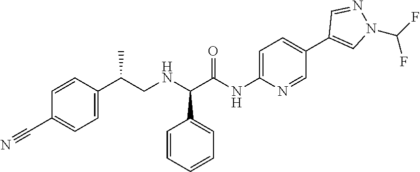 [SMILES: C[C@H](CN[C@@H](C(=O)Nc1ccc(-c2cnn(C(F)F)c2)cn1)c1ccccc1)c1ccc(C#N)cc1]

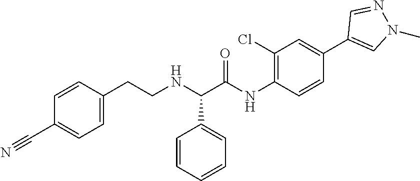 [SMILES: Cn1cc(-c2ccc(NC(=O)[C@@H](NCCc3ccc(C#N)cc3)c3ccccc3)c(Cl)c2)cn1]

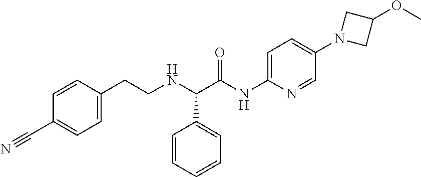 [SMILES: COC1CN(c2ccc(NC(=O)[C@@H](NCCc3ccc(C#N)cc3)c3ccccc3)nc2)C1]

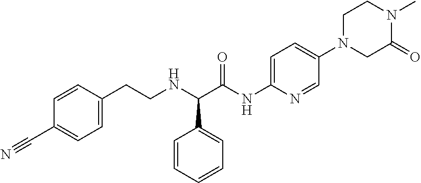 [SMILES: CN1CCN(c2ccc(NC(=O)[C@H](NCCc3ccc(C#N)cc3)c3ccccc3)nc2)CC1=O]

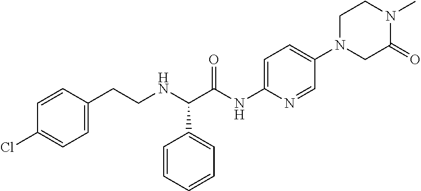 [SMILES: CN1CCN(c2ccc(NC(=O)[C@@H](NCCc3ccc(Cl)cc3)c3ccccc3)nc2)CC1=O]

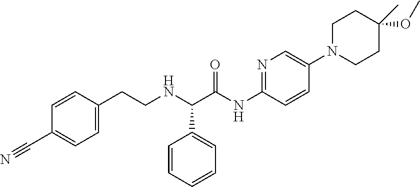

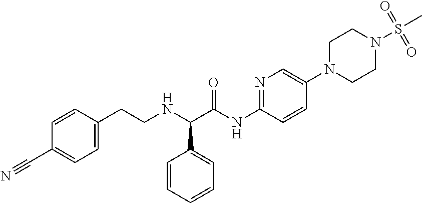 [SMILES: CS(=O)(=O)N1CCN(c2ccc(NC(=O)[C@H](NCCc3ccc(C#N)cc3)c3ccccc3)nc2)CC1]

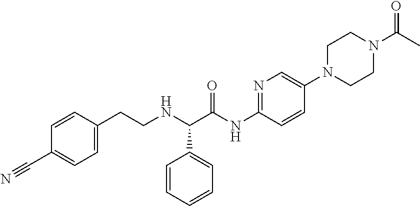 [SMILES: CC(=O)N1CCN(c2ccc(NC(=O)[C@@H](NCCc3ccc(C#N)cc3)c3ccccc3)nc2)CC1]

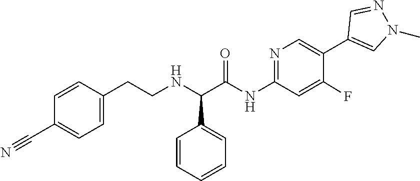 [SMILES: Cn1cc(-c2cnc(NC(=O)[C@H](NCCc3ccc(C#N)cc3)c3ccccc3)cc2F)cn1]

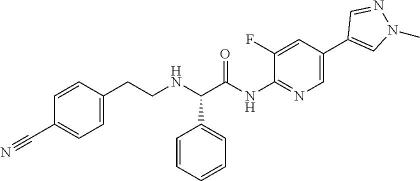 [SMILES: Cn1cc(-c2cnc(NC(=O)[C@@H](NCCc3ccc(C#N)cc3)c3ccccc3)c(F)c2)cn1]

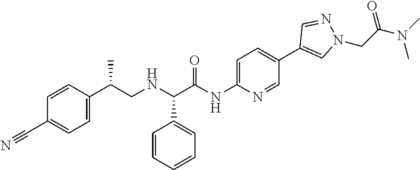 [SMILES: C[C@H](CN[C@H](C(=O)Nc1ccc(-c2cnn(CC(=O)N(C)C)c2)cn1)c1ccccc1)c1ccc(C#N)cc1]

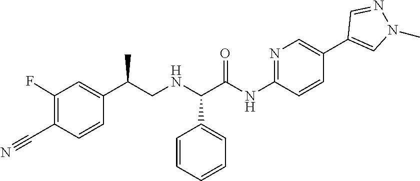 [SMILES: C[C@@H](CN[C@H](C(=O)Nc1ccc(-c2cnn(C)c2)cn1)c1ccccc1)c1ccc(C#N)c(F)c1]

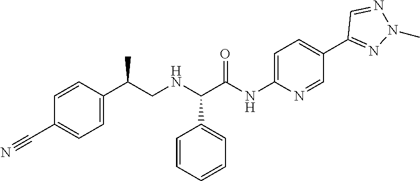 [SMILES: C[C@@H](CN[C@H](C(=O)Nc1ccc(-c2cnn(C)n2)cn1)c1ccccc1)c1ccc(C#N)cc1]

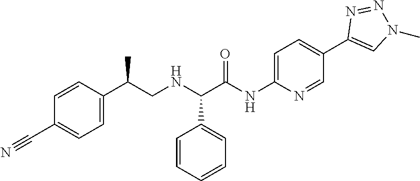 [SMILES: C[C@@H](CN[C@H](C(=O)Nc1ccc(-c2cn(C)nn2)cn1)c1ccccc1)c1ccc(C#N)cc1]

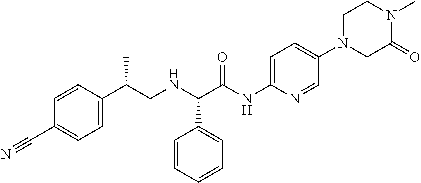 [SMILES: C[C@H](CN[C@H](C(=O)Nc1ccc(N2CCN(C)C(=O)C2)cn1)c1ccccc1)c1ccc(C#N)cc1]

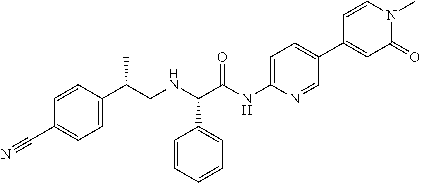 [SMILES: C[C@H](CN[C@H](C(=O)Nc1ccc(-c2ccn(C)c(=O)c2)cn1)c1ccccc1)c1ccc(C#N)cc1]

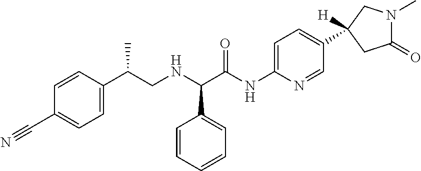 [SMILES: C[C@H](CN[C@@H](C(=O)Nc1ccc([C@H]2CC(=O)N(C)C2)cn1)c1ccccc1)c1ccc(C#N)cc1]

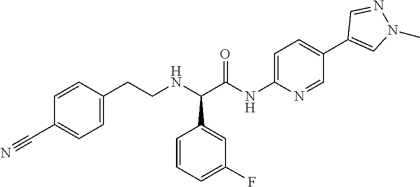 [SMILES: Cn1cc(-c2ccc(NC(=O)[C@H](NCCc3ccc(C#N)cc3)c3cccc(F)c3)nc2)cn1]

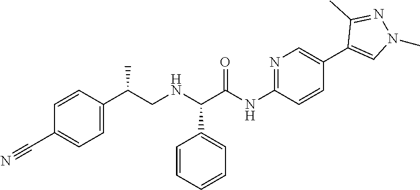 [SMILES: Cc1nn(C)cc1-c1ccc(NC(=O)[C@@H](NC[C@@H](C)c2ccc(C#N)cc2)c2ccccc2)nc1]

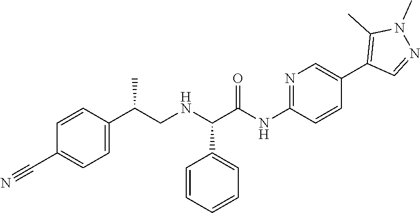 [SMILES: Cc1c(-c2ccc(NC(=O)[C@@H](NC[C@@H](C)c3ccc(C#N)cc3)c3ccccc3)nc2)cnn1C]

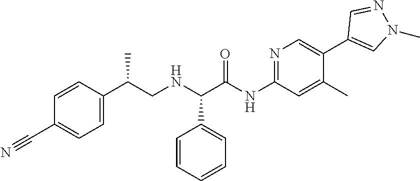 [SMILES: Cc1cc(NC(=O)[C@@H](NC[C@@H](C)c2ccc(C#N)cc2)c2ccccc2)ncc1-c1cnn(C)c1]

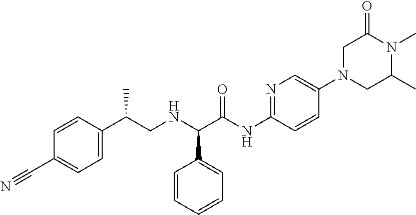 [SMILES: CC1CN(c2ccc(NC(=O)[C@H](NC[C@@H](C)c3ccc(C#N)cc3)c3ccccc3)nc2)CC(=O)N1C]

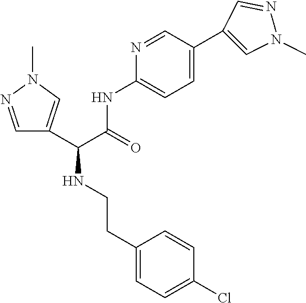 [SMILES: Cn1cc(-c2ccc(NC(=O)[C@@H](NCCc3ccc(Cl)cc3)c3cnn(C)c3)nc2)cn1]

or a pharmaceutically acceptable salt thereof.

### [Item 57]

The pharmaceutical composition of item 42, wherein the low molecular weight compound is a compound represented by formula (8)

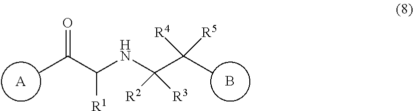

wherein
ring A is bicyclic heteroaryl optionally substituted with 1 to 4 substituents selected from R^{a};
ring B is aryl, heterocyclyl, or heteroaryl optionally substituted with 1 to 4 substituents selected from R^{b};
R¹ is C₁₋₆ alkyl, C₁₋₆ haloalkyl, C₂₋₆ alkenyl, —C₁₋₆ alkyl OR^{c}, —C₁₋₆ alkyl N(R^{d})₂, —C₁₋₆ alkyl C(O)OR^{d}, —C₁₋₆ alkyl OC₁₋₆ alkyl N(R^{d})₂, —C₁₋₆ alkyl SOR^{d}, —C₁₋₆ alkyl S(O)₂R^{d}, —C₁₋₆ alkyl SON(R^{d})₂, —C₁₋₆ alkyl SO₂N(R^{d})₂, —C₁₋₆ alkylcycloalkyl, —C₁₋₆ alkylheterocyclyl, —C₁₋₆ alkylheteroaryl, —C₁₋₆ alkylaryl, cycloalkyl, aryl, heteroaryl, or heterocyclyl (wherein each of the cycloalkyl, heterocyclyl, aryl, and heteroaryl, alone or for —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 groups selected from R^{c});
R², R³, R⁴, and R⁵ are each independently a hydrogen atom or C₁₋₆ alkyl (wherein the C₁₋₆ alkyl is optionally substituted with 1 to 2 substituents selected from a halogen atom, —C(O)OR^{d}, —OC₁₋₆ alkyl N(R^{d})₂, —C₁₋₆ alkyl N(R^{d})₂, —N(R^{d})—, —NR^{d}C₁₋₆ alkyl OR^{d}, —SOR^{d}, —S(O)₂R^{d}, —SON(R^{d})₂, —SO₂N(R^{d})₂, cycloalkyl, heterocyclyl, heteroaryl, and aryl);
R^{a}, R^{b}, and R^{c} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O) OR^{d}, —C(O)N(R^{d})₂, —C(O)NR^{d}C₁₋₆ alkyl OR^{d}, —OC₁₋₆ alkyl N(R^{d}), C₁₋₆ alkyl C(O)N(R^{d})₂, —C₁₋₆ alkyl N(R^{d})₂, —N(R^{d}), —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)—R^{d}, —SON(R^{d})₂, —SO₂N(R^{d})₂, —SF₅, —O— cycloalkyl, —O-heterocyclyl, —O—C₁₋₄ alkyl-aryl, —C₁₋₆ alkylcycloalkyl, —C₁₋₆ alkylaryl, —C₁₋₆ alkylheteroaryl, —C₁₋₆ alkylheterocyclyl, cycloalkyl, heterocyclyl, heteroaryl, or aryl (wherein each of the cycloalkyl, heterocyclyl, aryl, and heteroaryl, alone or attached to —O-cycloalkyl, —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 of a halogen atom, oxo, C₁₋₆ alkyl, C₁₋₆ haloalkyl, C₁₋₆ alkoxy, C₁₋₆ haloalkoxy, —N(R^{d})₂, —C(O)R^{d}, and —C₁₋₆ alkyl OR^{d});
R^{d} is each independently a hydrogen atom, heterocyclyl, C₁₋₆ haloalkyl, or C₁₋₆ alkyl, wherein the heterocyclyl is optionally substituted with 1 to 2 substituents selected from C₁₋₄ haloalkyl and C₁₋₄ alkyl, and the C₁₋₆ alkyl is optionally substituted with SO₂C₁₋₄ alkyl or heterocyclyl (wherein the group is optionally substituted with oxo);
wherein the compound is not 4-(2-((2-(1H-indol-3-yl)-2-oxo-1-phenylethyl)amino)ethyl)benzenesulfonamide, 4-[2-[[2-(7-ethyl-1H-indol-3-yl)-2-oxo-1-phenylethyl]amino]ethyl]benzenesulfonamide, 2-[[2-(3,4-dimethoxyphenyl)ethyl]amino]-1-(1H-indol-3-yl)-2-phenylethanone, or a salt thereof,
   or a prodrug thereof or a pharmaceutically acceptable salt thereof.

### [Item 58]

The pharmaceutical composition of item 42, wherein the low molecular weight compound is a compound represented by

TABLE 29

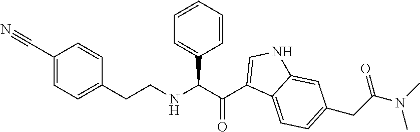 [SMILES: CN(C)C(=O)Cc1ccc2c(C(=O)[C@@H](NCCc3ccc(C#N)cc3)c3ccccc3)c[nH]c2c1]

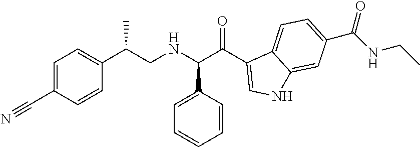 [SMILES: CCNC(=O)c1ccc2c(C(=O)[C@H](NC[C@@H](C)c3ccc(C#N)cc3)c3ccccc3)c[nH]c2c1]

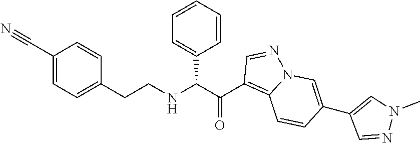 [SMILES: Cn1cc(-c2ccc3c(C(=O)[C@H](NCCc4ccc(C#N)cc4)c4ccccc4)cnn3c2)cn1]

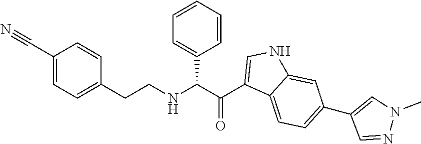 [SMILES: Cn1cc(-c2ccc3c(C(=O)[C@H](NCCc4ccc(C#N)cc4)c4ccccc4)c[nH]c3c2)cn1]

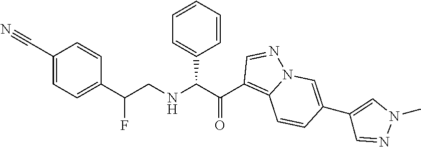 [SMILES: Cn1cc(-c2ccc3c(C(=O)[C@H](NCC(F)c4ccc(C#N)cc4)c4ccccc4)cnn3c2)cn1]

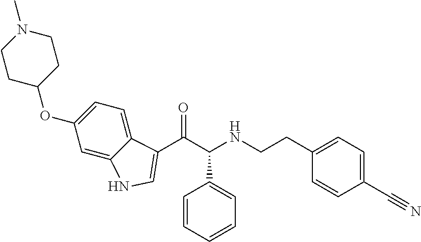 [SMILES: CN1CCC(Oc2ccc3c(C(=O)[C@H](NCCc4ccc(C#N)cc4)c4ccccc4)c[nH]c3c2)CC1]

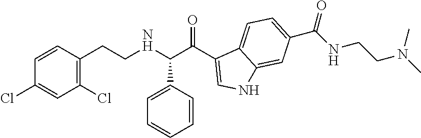 [SMILES: CN(C)CCNC(=O)c1ccc2c(C(=O)[C@@H](NCCc3ccc(Cl)cc3Cl)c3ccccc3)c[nH]c2c1]

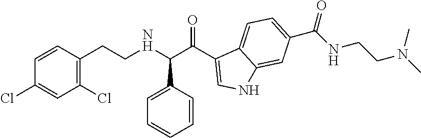 [SMILES: CN(C)CCNC(=O)c1ccc2c(C(=O)[C@H](NCCc3ccc(Cl)cc3Cl)c3ccccc3)c[nH]c2c1]

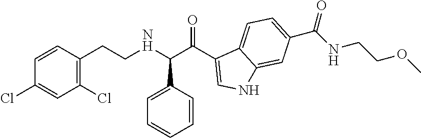 [SMILES: COCCNC(=O)c1ccc2c(C(=O)[C@H](NCCc3ccc(Cl)cc3Cl)c3ccccc3)c[nH]c2c1]

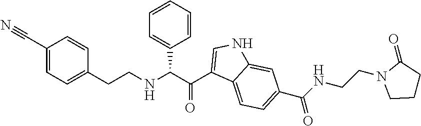 [SMILES: N#Cc1ccc(CCN[C@@H](C(=O)c2c[nH]c3cc(C(=O)NCCN4CCCC4=O)ccc23)c2ccccc2)cc1]

or a pharmaceutically acceptable salt thereof.

### [Item 59]

The pharmaceutical composition of item 42, wherein the low molecular weight compound is a compound represented by formula (9)

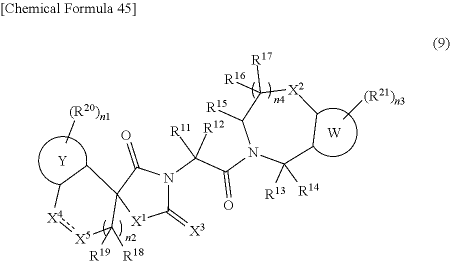

wherein
X¹ is independently —O—, —NR¹—, or —S—;
R¹ is independently a hydrogen atom, C₁₋₆, alkyl, or C₃₋₆ cycloalkyl;
X² is independently —C(R²)(R³)—, —O—, —N(R⁴)—, or —S(O)ₙ₁—;
R² and R³ are each independently a hydrogen atom, a deuterium atom, C₁₋₆ alkyl, C₃₋₆ haloalkyl, or C₃₋₈ cycloalkyl;
R⁴ is each independently a hydrogen atom, C₁₋₆ alkyl, C₃₋₆ cycloalkyl, —C(═O)(C₁₋₆ alkyl), —S(O)₂(C₁₋₆ alkyl), —C(═O)(C₃₋₆ cycloalkyl), or —S(O)₂(C₃₋₈ cycloalkyl);
X⁴ are each independently O or NH;

[symbol]

[Chemical Formula 46]

is a single bond or a double bond;
wherein if

[symbol]

[Chemical Formula 47]

is a single bond, X⁴ is independently —C(R⁵)(R⁶)—, —O—, —C(═O)—, —NR⁷—, or —S(O)ₙ₁—;
wherein if

[symbol]

[Chemical Formula 48]

is a single bond, X⁵ is independently —C(R⁸)(R⁹)—, —O—, —C(═O)—, —NR¹⁰—, —S(O)ₙ₁—, or a direct bond;
wherein if

[symbol]

[Chemical Formula 49]

is a double bond, X⁴ is independently —C(R⁵)—;
wherein if

[symbol]

[Chemical Formula 50]

is a double bond, X⁴ is independently —C(R⁵)—;
R⁵ and R⁶ are each independently a hydrogen atom, OH, a halogen atom, CN, C₁₋₆ alkyl, C₁₋₆ haloalkyl, C₂₋₆ cycloalkyl, or C₁₋₆ alkoxy;
R⁸ and R⁹ are each independently a hydrogen atom, OH, a halogen atom, or C₁₋₆ alkyl;
R⁷ is each independently a hydrogen atom, C₁₋₆ alkyl, or C₃₋₆ cycloalkyl;
R¹⁰ is each independently a hydrogen atom, C₁₋₆ alkyl, or C₃₋₆ cycloalkyl;
Y is independently a C₆₋₁₀ aromatic ring or a C₅₋₁₀ heteroaromatic ring (wherein the group is each independently unsubstituted, or optionally substituted with 1 to 2 R²⁰);
R¹¹ and R¹² are each independently a hydrogen atom, a deuterium atom, C₁₋₆ alkyl, C₁₋₆ haloalkyl, or C₃₋₆ cycloalkyl;
R¹³ and R¹⁴ are each independently a hydrogen atom, a deuterium atom, C₁₋₆ alkyl, C₁₋₆ haloalkyl, or C₃₋₆ cycloalkyl;
R¹⁶ and R¹⁷ are each independently a hydrogen atom, a deuterium atom, C₁₋₆ alkyl, C₁₋₆ haloalkyl, or C₃₋₆ cycloalkyl;
R¹⁸ and R¹⁹ are each independently a hydrogen atom, a halogen atom, or C₁₋₆ alkyl;
R¹⁵ is each independently a hydrogen atom, C₁₋₆ alkyl substituted with 0 to 2 R³, C₁₋₆ haloalkyl, or M³;
wherein R^{a} is independently C₁₋₆ alkyl, C₂₋₆ alkenyl, C₂₋₆ alkynyl, a halogen atom, C₁₋₆ haloalkyl, C₁₋₆ haloalkoxy, —CN, hydroxyl, —OMe, —SMe, —S(O)₂M^{e}, —C(O)NM^{f}M^{g}, —NM^{f}M^{g}, —N(M^{e})C(O)M^{h}, —N(M^{e})S(O)₂M^{h}, —N(M^{e})C(O)OM^{h}, —N(M^{e})C(O)NM^{f}M^{g}, or M^{b};
R²⁰ is independently a hydrogen atom, a halogen atom, —OH, —CN, —COOH, C₁₋₆ alkyl, C₁₋₆ alkoxy, C₁₋₆ haloalkoxy, C₁₋₁₀ alkoxyalkyl, C₄₋₂₀ alkoxyalkylalkynyl, C₂₋₁₀ haloalkoxyalkyl, C₁₋₆ hydroxyalkyl, C₃₋₁₀ hydroxyalkylalkynyl, C₂₋₁₀ hydroxyalkynyl, —B(R^{b})(R^{d}), —S(O)ₙ₁R^{c}, —N(R^{c})₂, —C(═O)N(R^{c})₂, —NHC(═O)R^{c}, —NHC(═O)OR^{c}, —NHC(═O)C(═O)N(R^{c})₂, —NHC(═O)C(═O)OR^{c}, —NHC(═O)N(R^{c})₂, —NHC(═O)NR^{c}C(═O)N(R^{c})₂, —NHC(═O)NRCS(O)₂OR^{c}, —NHC(═O)NR^{c}S(O)₂N(R^{c})₂, —NHC(═S)N(R^{c})₂, —NHC(═NC≡N)NR^{c}, —NHC(═NC≡N)SR^{c}, —NHS(O)ₙ₁R^{c}, M^{c}, —(C₁₋₆ alkylene)-B(R^{b})(R^{d}), —(C₁₋₆ alkylene)-S(O)ₙ₁R^{c}, —(C₁₋₆ alkylene)-N(R^{c})₂, —(C₁₋₆ alkylene)-C(═O)N(R^{c})₂, —(C₁₋₆ alkylene)-NHC(═O)R^{c}, —(C₁₋₆ alkylene)-NHC(═O)OR^{c}, —(C₁₋₆ alkylene)-NHC(═O)C(═O)N(R^{c})₂, —(C₁₋₆ alkylene)-NHC(═O)C(═O)OR^{c}, —(C₁₋₆ alkylene)-NHC(═O)N(R^{c})₂, —(C₁₋₆ alkylene)-NHC(═O)NR^{c}C(═O)N(R^{c})₂, —(C₁₋₆ alkylene)-NHC(═O)NRCS(O)₂OR^{c}, —(C₁₋₆ alkylene)-NHC(═O)NR^{c}S(O)₂N(R^{c})₂, —(C₁₋₆ alkylene)-NHC(═S)N(R^{c})₂, —(C₁₋₆ alkylene)-NHC(═NC≡N) NR^{c}, (C₁₋₆ alkylene)-NHC(═NC≡N)SR^{c}, —(C₁₋₆ alkylene)-NHS(O)ₙ₁R^{c}, —(C₁₋₆ alkylene)-M^{c}, —CH═CH—(C₁₋₆ alkyl), —CH═CH-M^{c}, —OM^{c}, —SM^{c}, or —N(R^{c})M^{c};
R^{b} and R^{d} are each independently a hydrogen atom, hydroxyl, or C₁₋₆ alkyl;
R^{c} is each independently a hydrogen atom, C₁₋₆ alkyl, C₆-10 aryl, 5- to 10-membered cyclic heteroaryl, a 3- to 10-membered cyclic non-aromatic heterocyclic group, C₃₋₁₀ cycloalkyl, or C₅₋₁₀ cycloalkenyl (wherein the group is each independently not substituted, or optionally substituted with 1 to 2 substituents selected from amino, hydroxy, methoxy, C₁₋₆ alkyl, C₃₋₁₀ cycloalkyl, or CN);
M^{a}, M^{b}, and M^{c} are each independently C₆₋₁₀ aryl, C₅₋₁₀ heteroaryl, a C₃₋₁₀ non-aromatic heterocyclic group, C₃₋₁₀ cycloalkyl, or C₃₋₁₀ cycloalkenyl (wherein the groups are each independently not substituted, or optionally substituted with 1 to 2 M^{d});
M^{d} is each independently C₁₋₆ alkyl, C₂₋₆ alkenyl, C₂₋₆ alkynyl, halogen, C₁₋₆ haloalkyl, —CN, oxo, —OM^{e}, —OC(O)M^{h}, —OC(O)NM^{f}M^{e}, —SM^{e}, —S(O)₂M^{e}, —S(O)₂NM^{f}M^{g}, —C(O)M^{e}, —C(O)-5- to 10-membered monocyclic cycloheteroaryl, —C(O)-5- to 10-membered monocyclic heteroaryl, —C(O)OM^{e}, —C(O)NM^{f}M^{g}, —NM^{f}M^{g}, —N(M^{e})C(O)M^{h}, —N(M^{e})S(O)₂M^{h}, —N(M^{e})C(O)OM^{h}, —N(M^{e})C(O)NM^{f}M^{g}, —(C₁₋₆ alkylene) OW, —(C₁₋₆ alkylene)-OC(O)M^{h}, —(C₁₋₆ alkylene)-OC(O)NM^{f}M^{g}, —(C₁₋₆ alkylene)-S(O)₂M^{e}, —(C₁₋₆ alkylene)-S(O)₂NM^{f}M^{g}, —(C₁₋₆ alkylene)-C(O)M^{e}, —(C₁₋₆ alkylene)-C(O)OM^{e}, —(C₁₋₆ alkylene)-C(O)NM^{f}M^{g}, —(C₁₋₆ alkylene)-NM^{f}M^{g}, —(C₁₋₆ alkylene)-N(M^{e})C(O)M^{h}, —(C₁₋₆ alkylene)-N(M^{e})S(O)₂M^{h}, —(C₁₋₆ alkylene)-N(M^{e})C(O)OM^{h}, —(C₁₋₆ alkylene)-N(M)C(O)NM^{f}M^{g}, or (C₁₋₆ alkylene)-CN;
W is independently a C₆₋₁₀ aromatic ring or a C₅₋₁₀ heteroaromatic ring (wherein the group is independently not substituted, or optionally substituted with 1 to 3 R²¹);
R²¹ is each independently C₁₋₆ alkyl, a halogen atom, C₁₋₆ haloalkyl, C₁₋₆ haloalkoxy, C₃₋₆ cycloalkyl, —OM^{e}, —OC(O)M^{h}, —OC(O)NM^{f}M^{g}, —SM^{e}, —S(O)₂M^{e}, —S(O)₂NM^{f}M^{g}, —C(O)M^{e}, —C(O)OM^{e}, —C(O)NM^{f}M^{g}, —N(M^{e})C(O)M^{h}, —N(M^{e})S(O)₂M^{h}, —N(M^{e})C(O)OM^{h}, or —N(M^{e})C(O)NM^{f}M^{g};
M^{e}, M^{f}, and M^{g} are each independently a hydrogen atom, C₁₋₆ alkyl, C₁₋₆ haloalkyl, or C₃₋₆ cycloalkyl;
M^{h} is each independently C₁₋₆ alkyl, C₁₋₆ haloalkyl, or C₃₋₆ cycloalkyl;
n1 and n2 are, for each instance, independently 0, 1, or 2; and
n3 and n4 are, for each instance, independently 0, 1, 2, or 3,
   or a prodrug thereof or a pharmaceutically acceptable salt thereof.

### [Item 60]

The pharmaceutical composition of item 42, wherein the low molecular weight compound is a compound represented by the following (Table 9)

TABLE 30

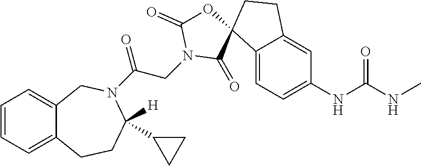 [SMILES: CNC(=O)Nc1ccc2c(c1)CC[C@@]21OC(=O)N(CC(=O)N2Cc3ccccc3CC[C@H]2C2CC2)C1=O]

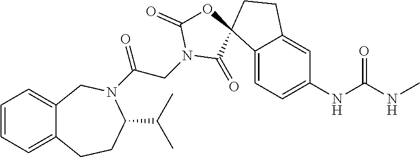 [SMILES: CNC(=O)Nc1ccc2c(c1)CC[C@]21OC(=O)N(CC(=O)N2Cc3ccccc3CC[C@H]2C(C)C)C1=O]

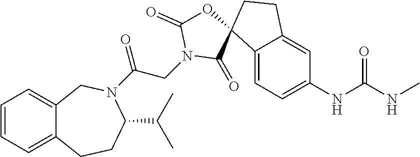 [SMILES: CNC(=O)Nc1ccc2c(c1)CC[C@@]21OC(=O)N(CC(=O)N2Cc3ccccc3CC[C@H]2C(C)C)C1=O]

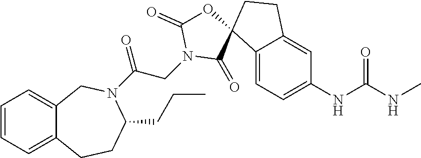 [SMILES: CCC[C@@H]1CCc2ccccc2CN1C(=O)CN1C(=O)O[C@@]2(CCc3cc(NC(=O)NC)ccc32)C1=O]

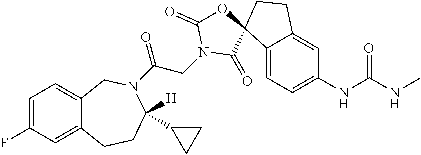 [SMILES: CNC(=O)Nc1ccc2c(c1)CC[C@]21OC(=O)N(CC(=O)N2Cc3ccc(F)cc3CC[C@H]2C2CC2)C1=O]

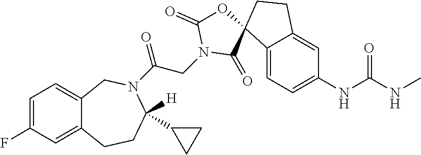 [SMILES: CNC(=O)Nc1ccc2c(c1)CC[C@@]21OC(=O)N(CC(=O)N2Cc3ccc(F)cc3CC[C@H]2C2CC2)C1=O]

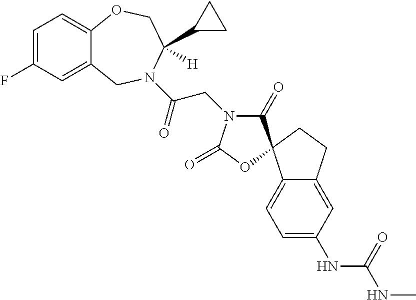 [SMILES: CNC(=O)Nc1ccc2c(c1)CC[C@@]21OC(=O)N(CC(=O)N2Cc3cc(F)ccc3OC[C@H]2C2CC2)C1=O]

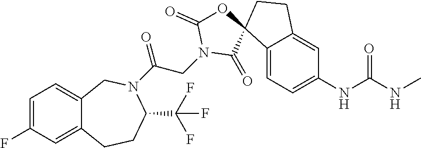 [SMILES: CNC(=O)Nc1ccc2c(c1)CC[C@]21OC(=O)N(CC(=O)N2Cc3ccc(F)cc3CC[C@H]2C(F)(F)F)C1=O]

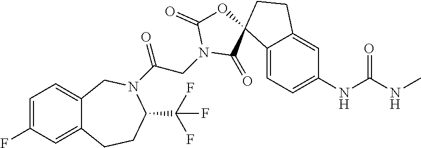 [SMILES: CNC(=O)Nc1ccc2c(c1)CC[C@@]21OC(=O)N(CC(=O)N2Cc3ccc(F)cc3CC[C@H]2C(F)(F)F)C1=O]

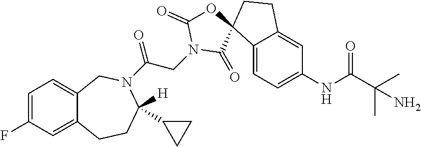 [SMILES: CC(C)(N)C(=O)Nc1ccc2c(c1)CC[C@]21OC(=O)N(CC(=O)N2Cc3ccc(F)cc3CC[C@H]2C2CC2)C1=O]

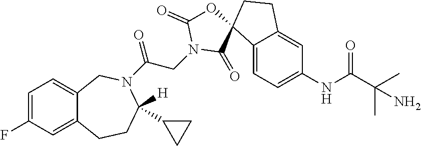 [SMILES: CC(C)(N)C(=O)Nc1ccc2c(c1)CC[C@@]21OC(=O)N(CC(=O)N2Cc3ccc(F)cc3CC[C@H]2C2CC2)C1=O]

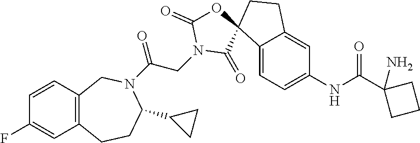 [SMILES: NC1(C(=O)Nc2ccc3c(c2)CC[C@]32OC(=O)N(CC(=O)N3Cc4ccc(F)cc4CC[C@H]3C3CC3)C2=O)CCC1]

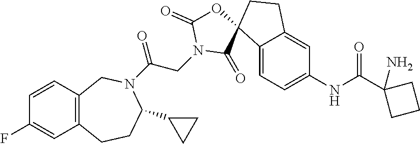 [SMILES: NC1(C(=O)Nc2ccc3c(c2)CC[C@@]32OC(=O)N(CC(=O)N3Cc4ccc(F)cc4CC[C@H]3C3CC3)C2=O)CCC1]

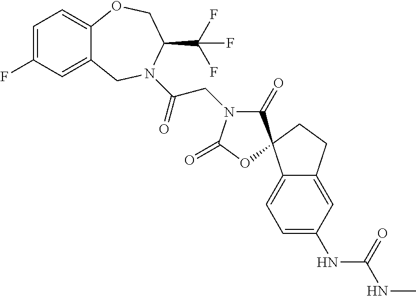 [SMILES: CNC(=O)Nc1ccc2c(c1)CC[C@@]21OC(=O)N(CC(=O)N2Cc3cc(F)ccc3OC[C@H]2C(F)(F)F)C1=O]

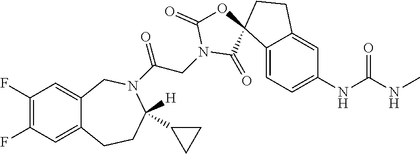 [SMILES: CNC(=O)Nc1ccc2c(c1)CC[C@]21OC(=O)N(CC(=O)N2Cc3cc(F)c(F)cc3CC[C@H]2C2CC2)C1=O]

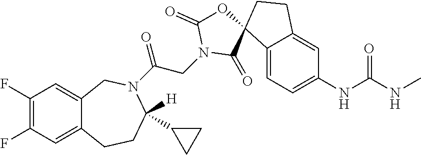 [SMILES: CNC(=O)Nc1ccc2c(c1)CC[C@@]21OC(=O)N(CC(=O)N2Cc3cc(F)c(F)cc3CC[C@H]2C2CC2)C1=O]

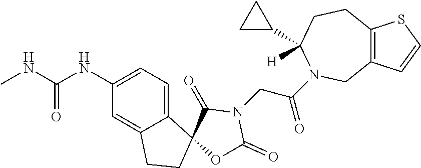 [SMILES: CNC(=O)Nc1ccc2c(c1)CC[C@@]21OC(=O)N(CC(=O)N2Cc3ccsc3CC[C@H]2C2CC2)C1=O]

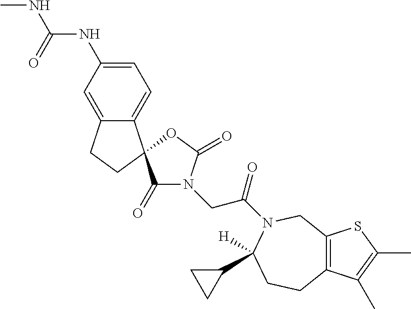

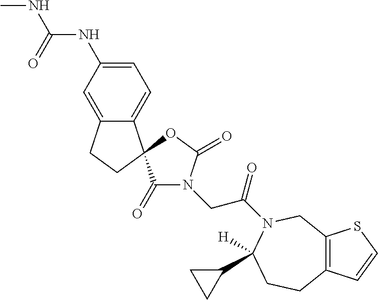 [SMILES: CNC(=O)Nc1ccc2c(c1)CC[C@]21OC(=O)N(CC(=O)N2Cc3sccc3CC[C@H]2C2CC2)C1=O]

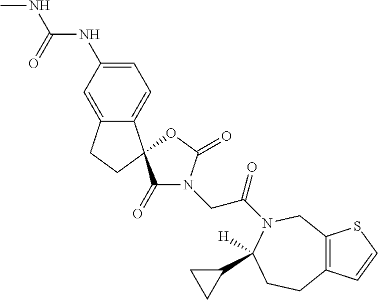 [SMILES: CNC(=O)Nc1ccc2c(c1)CC[C@@]21OC(=O)N(CC(=O)N2Cc3sccc3CC[C@H]2C2CC2)C1=O]

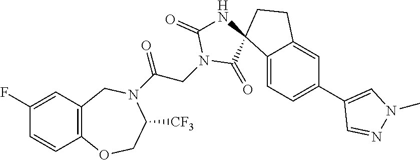 [SMILES: Cn1cc(-c2ccc3c(c2)CC[C@]32NC(=O)N(CC(=O)N3Cc4cc(F)ccc4OC[C@H]3C(F)(F)F)C2=O)cn1]

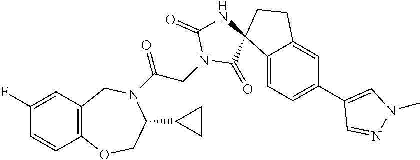 [SMILES: Cn1cc(-c2ccc3c(c2)CC[C@]32NC(=O)N(CC(=O)N3Cc4cc(F)ccc4OC[C@H]3C3CC3)C2=O)cn1]

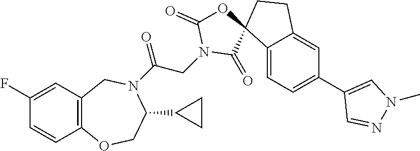 [SMILES: Cn1cc(-c2ccc3c(c2)CC[C@]32OC(=O)N(CC(=O)N3Cc4cc(F)ccc4OC[C@H]3C3CC3)C2=O)cn1]

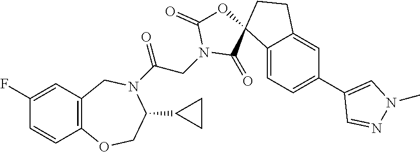 [SMILES: Cn1cc(-c2ccc3c(c2)CC[C@@]32OC(=O)N(CC(=O)N3Cc4cc(F)ccc4OC[C@H]3C3CC3)C2=O)cn1]

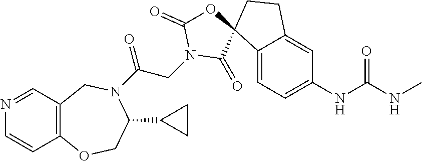 [SMILES: CNC(=O)Nc1ccc2c(c1)CC[C@]21OC(=O)N(CC(=O)N2Cc3cnccc3OC[C@H]2C2CC2)C1=O]

or a pharmaceutically acceptable salt thereof.

### [Item 61]

The pharmaceutical composition of item 42, wherein the low molecular weight compound is a compound represented by formula (10)

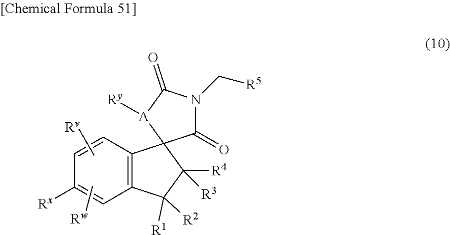

wherein
A is independently selected from O, N, and S;
R^{y} is absent, a hydrogen atom, alkyl, substituted alkyl, or alkenyl;
R^{y}, R^{w}, and R^{x} are each independently a hydrogen atom, a halogen atom, cyano, nitro, alkyl, substituted alkyl, alkenyl, alkynyl, cycloalkyl, substituted cycloalkyl, a heterocycle, a substituted heterocycle, aryl, substituted aryl, an aromatic heterocycle, a substituted aromatic heterocycle, substituted amide, substituted guanidino, substituted urea, amino, substituted amino, alkoxy, or substituted alkoxy;
R¹, R², R³, and R⁴ are each independently a hydrogen atom, alkyl, or a halogen atom;
wherein R¹ and R², R² and R³, or R³ and R⁴ together may from a ring;
R⁵ is alkyl, alkoxy, amino, substituted amino, amide, substituted amide, ester, carbonyl, a heterocycle, or a substituted heterocycle,
   or a prodrug thereof or a pharmaceutically acceptable salt thereof.

### [Item 62]

The pharmaceutical composition of item 42, wherein the low molecular weight compound is a compound represented by the following (Table 10)

TABLE 31

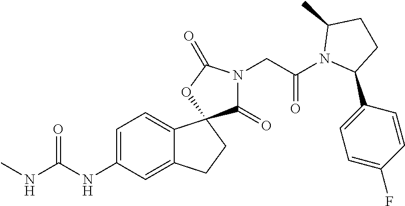 [SMILES: CNC(=O)Nc1ccc2c(c1)CC[C@@]21OC(=O)N(CC(=O)N2[C@@H](C)CC[C@H]2c2ccc(F)cc2)C1=O]

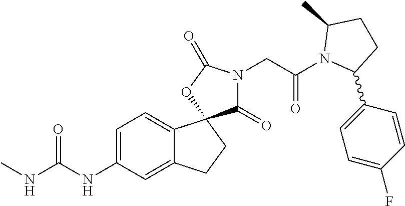 [SMILES: CNC(=O)Nc1ccc2c(c1)CC[C@@]21OC(=O)N(CC(=O)N2C(c3ccc(F)cc3)CC[C@@H]2C)C1=O]

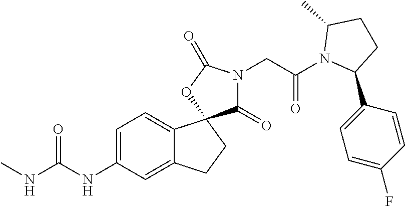 [SMILES: CNC(=O)Nc1ccc2c(c1)CC[C@@]21OC(=O)N(CC(=O)N2[C@H](C)CC[C@H]2c2ccc(F)cc2)C1=O]

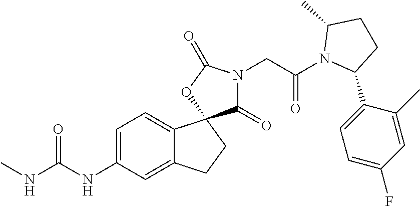

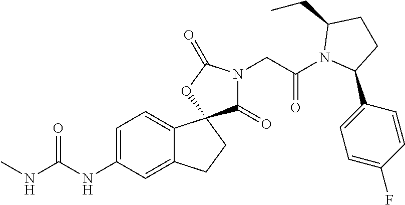 [SMILES: CC[C@H]1CC[C@@H](c2ccc(F)cc2)N1C(=O)CN1C(=O)O[C@@]2(CCc3cc(NC(=O)NC)ccc32)C1=O]

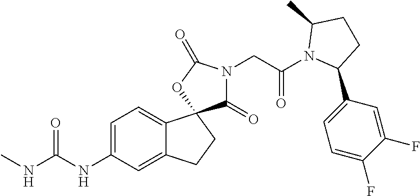 [SMILES: CNC(=O)Nc1ccc2c(c1)CC[C@@]21OC(=O)N(CC(=O)N2[C@@H](C)CC[C@H]2c2ccc(F)c(F)c2)C1=O]

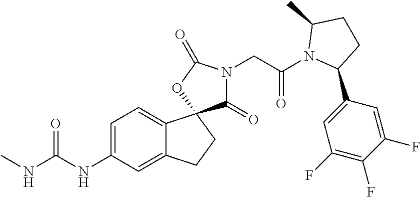 [SMILES: CNC(=O)Nc1ccc2c(c1)CC[C@@]21OC(=O)N(CC(=O)N2[C@@H](C)CC[C@H]2c2cc(F)c(F)c(F)c2)C1=O]

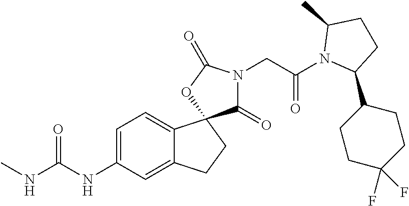 [SMILES: CNC(=O)Nc1ccc2c(c1)CC[C@@]21OC(=O)N(CC(=O)N2[C@@H](C)CC[C@H]2C2CCC(F)(F)CC2)C1=O]

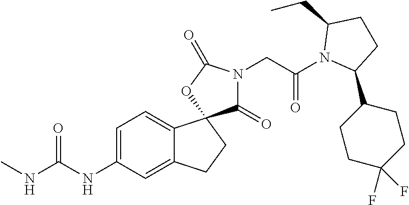 [SMILES: CC[C@H]1CC[C@@H](C2CCC(F)(F)CC2)N1C(=O)CN1C(=O)O[C@@]2(CCc3cc(NC(=O)NC)ccc32)C1=O]

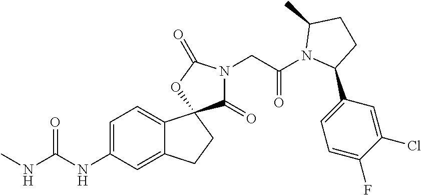 [SMILES: CNC(=O)Nc1ccc2c(c1)CC[C@@]21OC(=O)N(CC(=O)N2[C@@H](C)CC[C@H]2c2ccc(F)c(Cl)c2)C1=O]

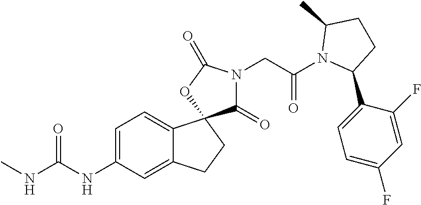 [SMILES: CNC(=O)Nc1ccc2c(c1)CC[C@@]21OC(=O)N(CC(=O)N2[C@@H](C)CC[C@H]2c2ccc(F)cc2F)C1=O]

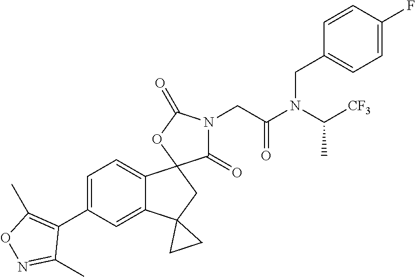 [SMILES: Cc1noc(C)c1-c1ccc2c(c1)C1(CC1)CC21OC(=O)N(CC(=O)N(Cc2ccc(F)cc2)[C@@H](C)C(F)(F)F)C1=O]

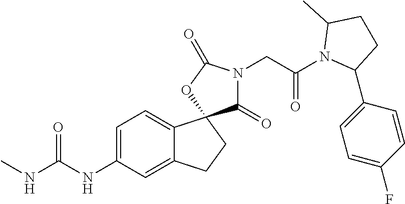 [SMILES: CNC(=O)Nc1ccc2c(c1)CC[C@@]21OC(=O)N(CC(=O)N2C(C)CCC2c2ccc(F)cc2)C1=O]

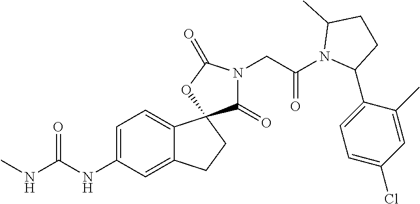

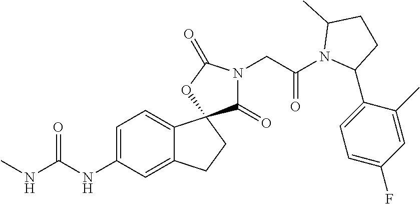

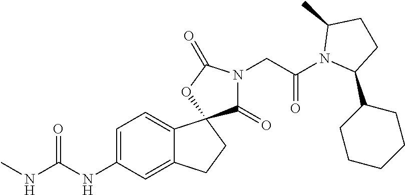 [SMILES: CNC(=O)Nc1ccc2c(c1)CC[C@@]21OC(=O)N(CC(=O)N2[C@@H](C)CC[C@H]2C2CCCCC2)C1=O]

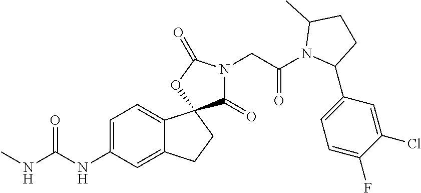 [SMILES: CNC(=O)Nc1ccc2c(c1)CC[C@@]21OC(=O)N(CC(=O)N2C(C)CCC2c2ccc(F)c(Cl)c2)C1=O]

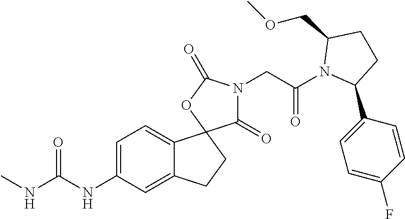 [SMILES: CNC(=O)Nc1ccc2c(c1)CCC21OC(=O)N(CC(=O)N2[C@@H](COC)CC[C@H]2c2ccc(F)cc2)C1=O]

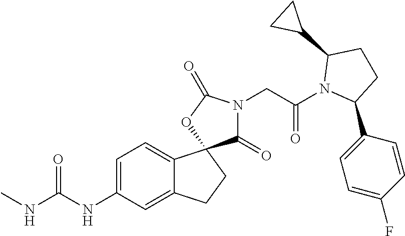 [SMILES: CNC(=O)Nc1ccc2c(c1)CC[C@@]21OC(=O)N(CC(=O)N2[C@@H](C3CC3)CC[C@H]2c2ccc(F)cc2)C1=O]

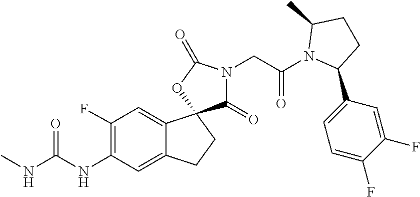 [SMILES: CNC(=O)Nc1cc2c(cc1F)[C@@]1(CC2)OC(=O)N(CC(=O)N2[C@@H](C)CC[C@H]2c2ccc(F)c(F)c2)C1=O]

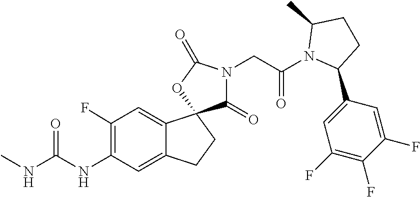 [SMILES: CNC(=O)Nc1cc2c(cc1F)[C@@]1(CC2)OC(=O)N(CC(=O)N2[C@@H](C)CC[C@H]2c2cc(F)c(F)c(F)c2)C1=O]

or a pharmaceutically acceptable salt thereof.

### [Item 63]

The pharmaceutical composition of item 42, wherein the low molecular weight compound is a compound represented by formula (11)

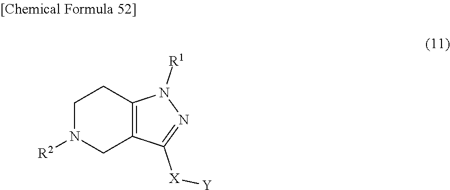

wherein
R¹ is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R¹ is optionally substituted with one or more R^{d};
R² is —C(O)—N(R^{e})₂, —S(O)—N(R^{e})₂, —S(O)₂—N(R^{e})₂, —C(O)—R^{e}, —C(O)—O—(R^{c}), —S(O)—R^{c}, or —S(O)₂—R^{c};
X is absent, —C(O), or C₁₋₃ alkyl;
Y is phenyl, a 9-membered bicyclic carbocyclic ring, a 10-membered bicyclic carbocyclic ring, a 9-membered bicyclic heterocycle, or a 10-membered bicyclic heterocycle;
wherein Y is optionally substituted with R^{a}, and Y is optionally further substituted with one or more R^{b}; or
X combined with Y is selected from the group consisting of

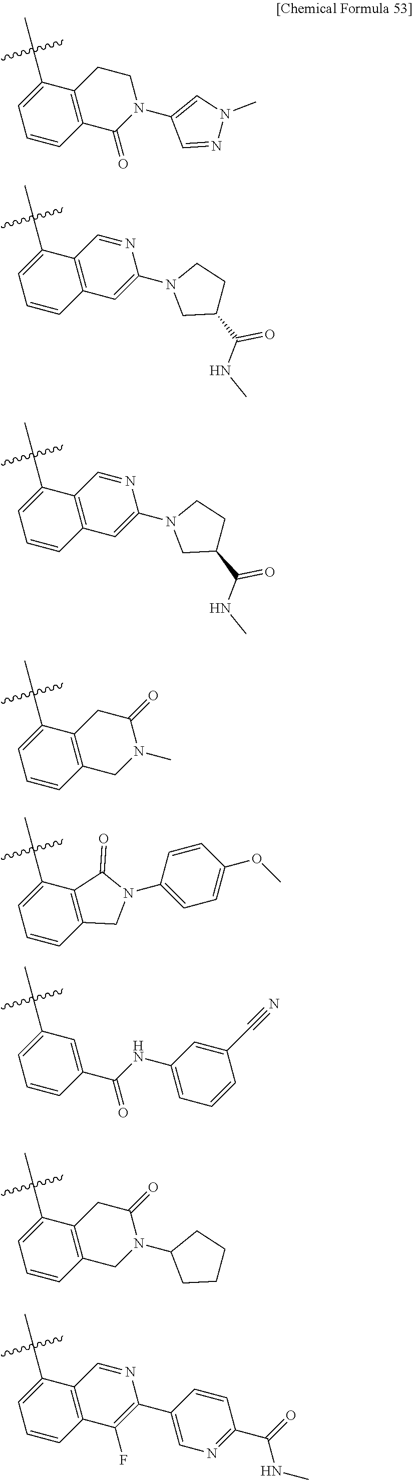

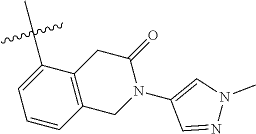 [SMILES: *c1cccc2c1CC(=O)N(c1cnn(C)c1)C2]

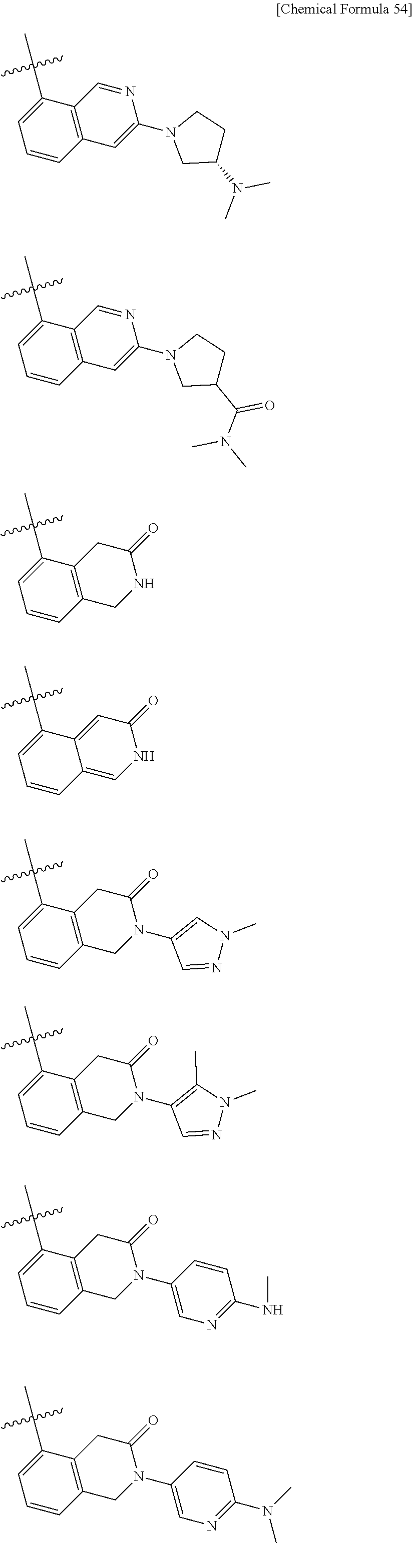

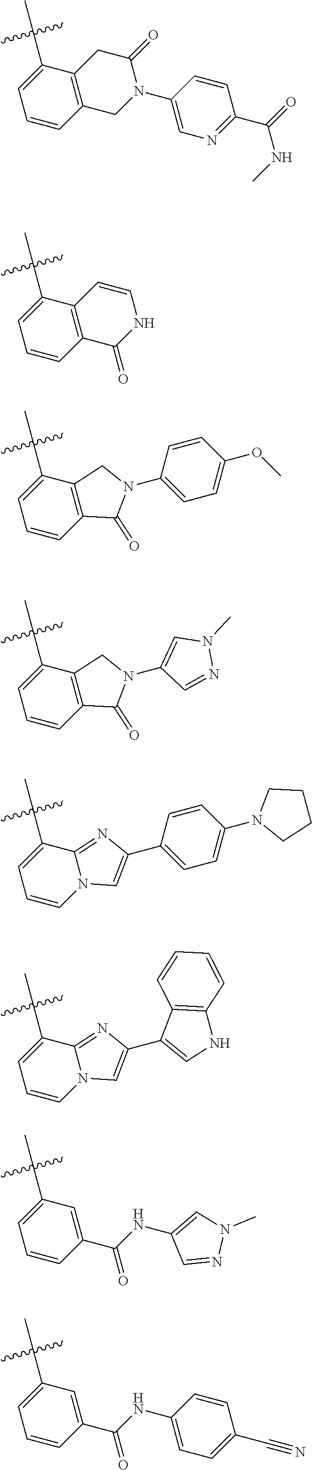

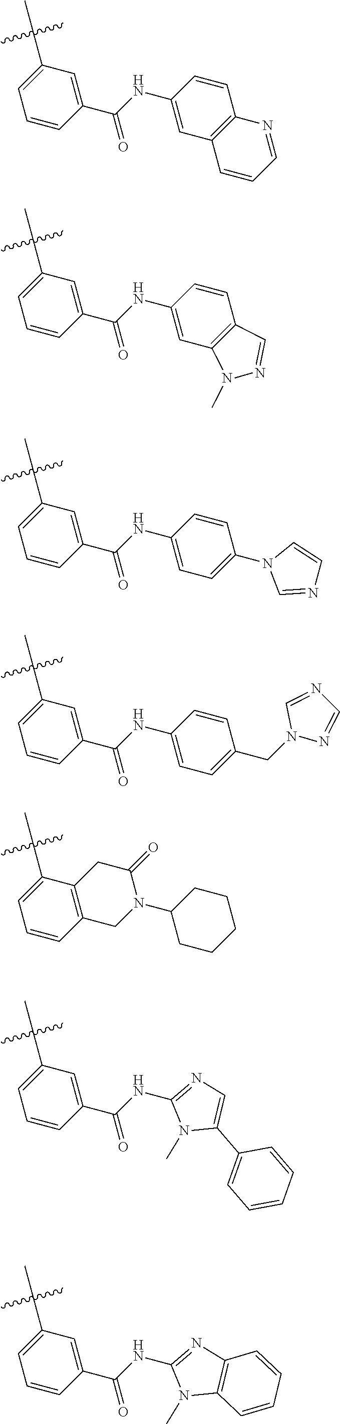

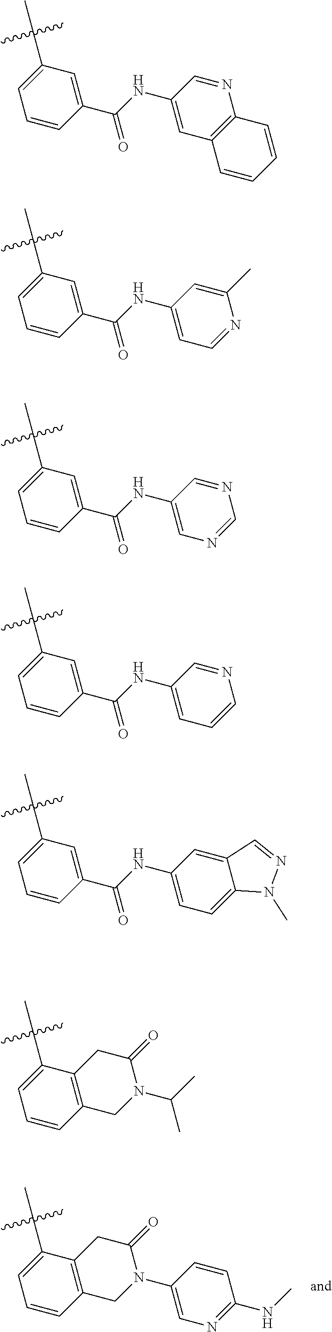

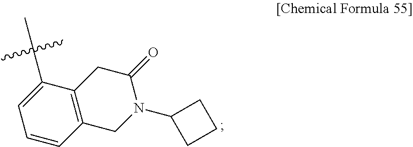 [SMILES: *c1cccc2c1CC(=O)N(C1CCC1)C2]

each R^{a} is independently selected from the group consisting of a 5-membered carbocyclic ring, a 6-membered carbocyclic ring, a 5-membered heterocycle, and a 6-membered heterocycle, wherein the 5-membered carbocyclic ring, 6-membered carbocyclic ring, 5-membered heterocycle, and 6-membered heterocycle are optionally substituted with one or more R^{c};
each R^{b} is independently selected from the group consisting of a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, —C(O)—N(R^{f})₂, —N(R^{f})C(O)—R^{f}, and C₁₋₄ alkanoyloxy, wherein each of C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₃ alkoxy, and C₁₋₃ alkyl that is optionally substituted with one or more groups independently selected from halogen;
R^{c} is independently selected from the group consisting of a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy, wherein each of C₁₋₄ alkyl, C₂₋₄ alkenyl, C₂₋₄ alkynyl, C₂₋₆ cycloalkyl, (C₂₋₆ cycloalkyl) C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₃ alkoxy, C₁₋₃ alkyl, and C₁₋₃ alkyl that is optionally substituted with one or more groups independently selected from halogen;
each R^{d} is independently selected from the group consisting of oxo, a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy, wherein each of C₁₋₄ alkyl, C₂₋₄ alkenyl, C₂₋₄ alkynyl, C₂₋₆ cycloalkyl, (C₂₋₆ cycloalkyl) C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₃ alkoxy, and C₁₋₃ alkyl that is optionally substituted with one or more groups independently selected from halogen;
each R^{e} is independently selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkyl, and C₁₋₃ alkyl that is optionally substituted with one or more groups independently selected from halogen;
each R^{f} is a hydrogen atom or C₁₋₄ alkyl; or
   selected from the group consisting of

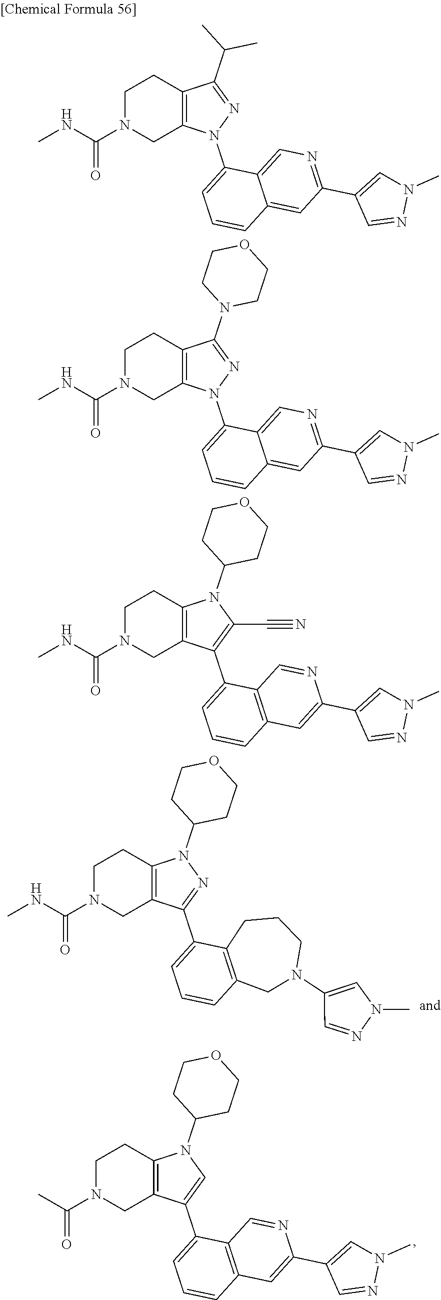

or a prodrug thereof or a pharmaceutically acceptable salt thereof.

### [Item 64]

The pharmaceutical composition of item 42, wherein the low molecular weight compound is a compound represented by the following (Table 11)

TABLE 32

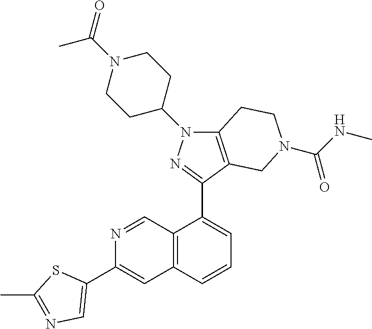

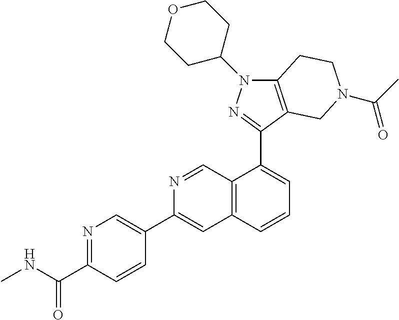 [SMILES: CNC(=O)c1ccc(-c2cc3cccc(-c4nn(C5CCOCC5)c5c4CN(C(C)=O)CC5)c3cn2)cn1]

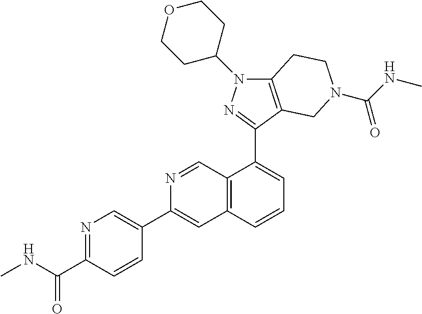

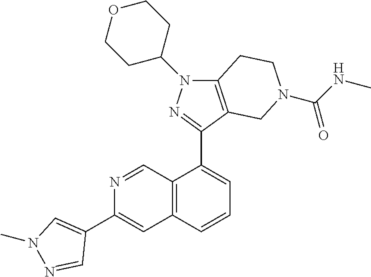

or a pharmaceutically acceptable salt thereof.

### [Item 65]

The pharmaceutical composition of item 42, wherein the low molecular weight compound is a compound represented by formula (12) or (13)

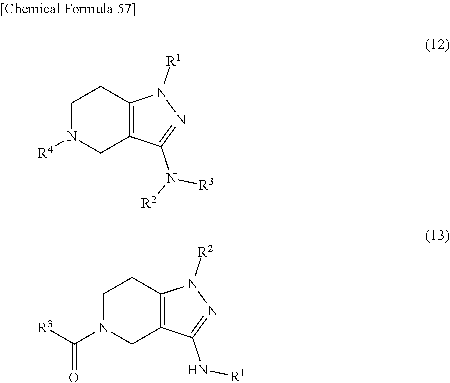

wherein
R¹ in formula (14) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R¹ is optionally substituted with one or more R^{b};
R² in formula (14) is selected from C₆₋₂₀ aryl, C₁₋₂₀ heteroaryl, —(C₆₋₂₀ aryl)-(C₁₋₂₀ heteroaryl), —(C₁₋₂₀ heteroaryl)-(C₆₋₂₀ aryl), and —(C₁₋₂₀ heteroaryl)-(C₁₋₂₀ heteroaryl), wherein each of C₆₋₂₀ aryl, C₁₋₂₀ heteroaryl, —(C₆₋₂₀ aryl)-(C₁₋₂₀ heteroaryl), and (C₁₋₂₀ heteroaryl)-(C₁₋₂₀ heteroaryl) is independently optionally substituted with one or more substituents independently selected from R^{c}, oxo, fluorine, chlorine, bromine, iodine, —NO₂, —N(R^{a})₂, —CN, —C(O)—N(R^{a})₂, —S(O)—N(R^{a})₂, —S(O)₂—N(R^{a})₂, —O—R^{a}, —S—R^{a}, —O—C(O)—R^{a}, —O—C(O)—O—R^{a}, —C(O)—R^{a}, —C(O)—O—R^{a}, —S(O)—R^{d}, —S(O)₂—R^{a}, —O—C(O)—N(R^{a})₂, —N(R^{a})—C(O)—OR^{a}, —N(R^{a})—C(O)—N(R^{a})₂, —N(R^{a})—C(O)—R^{a}, —N(R^{a})—S(O)—R^{a}, —N(R^{a})—S(O)₂—R^{a}, —N(R^{a})—S(O)—N(R^{a})₂, and —N(R^{a})—S(O)₂—N(R^{a})₂;
R³ in formula (14) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R³ is optionally substituted with one or more R^{c}; or
R² and R³ in formula (14), together with the nitrogen to which they are attached, form a 3- to 12-membered heterocycle optionally substituted with one or more R^{e};
R⁴ in formula (14) is C₁₋₄ alkyl, C₂₋₄ alkenyl, C₂₋₄ alkynyl, a 3- to 5-membered carbocyclic ring, a 3- to 5-membered heterocycle, —C(O)—N(R^{h})₂, —S(O)—N(R^{h})₂, —S(O)₂—N(R^{h})₂, —C(O)—R^{h}, —C(O)—OR^{h}, —S(O)—R^{h}, or —S(O)₂—R^{a}, wherein any of C₁₋₄ alkyl, C₂₋₄ alkenyl, C₂₋₄ alkynyl, 3- to 5-membered carbocyclic ring, and 3- to 5-membered heterocycle is optionally substituted with one or more substituents independently selected from fluorine, chlorine, bromine, iodine, a 3- to 5-membered carbocyclic ring, —C(O)—N(R^{h})₂, —S(O)—N(R^{h}), —S(O)—N(R^{h}), —O—R^{h}, —S—R^{h}, —O—C(O)—R^{h}, —O—C(O)—O—R^{h}, —C(O)—R^{h}, —C(O)—O—R^{h}, —S(O)—R^{h}, —S(O)₂—R^{h}, —O—C(O)—N(R^{h})₂, —N(R^{h})—C(O)—OR^{h}, —N(R^{h})—C(O)—N(R^{h}), —N(R^{h})—C(O)—R^{h}, —N(R^{h})—S(O)—R^{h}, —N(R^{h})—S(O)₂—R^{h}, —N(R^{h})—S(O)—N(R^{h})₂, and —N(R^{h})—S(O)—N(R^{h})₂;
each R^{a} in formula (14) is independently selected from a hydrogen atom, C₁₋₆ alkyl, C₂₋₆ alkenyl, C₂₋₆ alkynyl, a carbocyclic ring, and a heterocycle, wherein each of C₁₋₆ alkyl, C₂₋₆ alkenyl, C₂₋₆ alkynyl, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{a}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{b} in formula (14) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —O—C(O)—O—R^{c}, —C(O)—R^{c}, —C(O)—O—R^{c}, —S(O)—R^{c}, —S(O)₂—R^{c}, —O—C(O)—N(R^{c})₂, —N(R^{c})—C(O)—OR^{c}, —N(R^{c})—C(O)—N(R^{c})₂, —N(R^{c})—C(O)—R^{c}, —N(R^{c})—S(O)—R^{c}, —N(R^{c})—S(O)₂—R^{c}, —N(R^{c})—S(O)—N(R^{c})₂, and —N(R^{c})—S(O)₂—N(R^{c})₂, wherein any of C₁₋₆ alkyl, C₂₋₆ alkenyl, C₂₋₆ alkynyl, carbocyclic ring, heterocycle, aryl, and heteroaryl is optionally substituted with one or more groups independently selected from oxo, halogen, —NO, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —C(O)—R^{c}, —C(O)—O—R^{c}, —S(O)—R^{c}, —S(O)—R^{c}, —C(O)—N(R^{c})₂, —N(R^{c})—C(O)—R^{c}, —N(R^{c})—S(O)—R^{c}, —N(R^{c})—S(O)₂—R^{c}, and C₁₋₆ alkyl that is optionally substituted with one or more groups independently selected from oxo and halogen;
each R^{c} of formula (14) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —O—C(O)—R^{d}, —C(O)—R^{d}, —C(O)—O—R^{d}, —S(O)—R^{d}, —S(O)₂—R^{d}, —C(O)—N(R^{d})₂, —N(R^{d})—C(O)—R^{d}, —N(R^{d})—S(O)—R^{d}, —N(R^{d})—S(O)₂—R^{d}, and C₁₋₆ alkyl, and the carbocyclic ring and C₁₋₆ alkyl are optionally substituted with one or more groups independently selected from oxo, halogen, C₁₋₆ alkyl, cyano, —N(R^{d})₂, —O—R^{d}, a heterocycle, and a carbocyclic ring that is optionally substituted with one or more groups independently selected from halogen and C₁₋₆ alkyl;
each R^{d} in formula (14) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is independently optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or,
two R^{d}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{e} in formula (14) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{f})₂, —CN, —C(O)—N(R^{f})₂, —S(O)—N(R^{f})₂, —S(O)₂—N(R^{f})₂, —O—R^{f}, —S—R^{f}, —O—C(O)—R^{f}, —O—C(O)—O—R^{f}, —C(O)—R^{f}, —C(O)—O—R^{f}, —S(O)—R^{f}, —S(O)₂—R^{f}, —O—C(O)—N(R^{f})₂, —N(R^{f})—C(O)—OR^{f}, —N(R^{f})—C(O)—N(R^{f})₂, —N(R^{f})—C(O)—R^{f}, —N(R^{f})—S(O)—R^{f}, —N(R^{f})—S(O)₂—R^{f}, —N(R^{f})—S(O)—N(R^{f})₂, and —N(R^{f})—S(O)₂—N(R^{f})₂, wherein any of C₁₋₆ alkyl, C₂₋₆ alkenyl, C₂₋₆ alkynyl, carbocyclic ring, heterocycle, aryl, and heteroaryl may be substituted with one or more groups independently selected from oxo, halogen, —NO₂, —N(R^{f})₂, —CN, —C(O)—N(R^{f})₂, —S(O)—N(R^{f})₂, —S(O)₂—N(R^{f})₂, —O—R^{f}, —S—R^{f}, —O—C(O)—R^{f}, —C(O)—R^{f}, —C(O)—O—R^{f}, —S(O)—R^{f}, —S(O)₂—R^{f}, —C(O)—N(R^{f})₂, —N(R^{f})—C(O)—R^{f}, —N(R^{f})—S(O)—R^{f}, —N(R^{f})—S(O)₂—R^{f}, a carbocyclic ring, and C₁₋₆ alkyl that is optionally substituted with one or more groups independently selected from oxo and halogen;
each R^{f} in formula (I) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{g})₂, —CN, —C(O)—N(R^{g})₂, —S(O)—N(R^{g})₂, —S(O)₂—N(R^{e})₂, —O—R^{g}, —S—R^{g}, —O—C(O)—R^{g}, —C(O)—R^{g}, —C(O)—O—R^{g}, —S(O)—R^{g}, —S(O)₂—R^{g}, —C(O)—N(R^{g})₂, —N(R^{g})—C(O)—R^{g}, —N(R^{g})—S(O)—R^{g}, —N(R^{g})—S(O)₂—R^{g}, and C₁₋₆ alkyl, and the carbocyclic ring and C₁₋₆ alkyl are optionally substituted with one or more groups independently selected from oxo, halogen, C₁₋₆ alkyl, cyano, —N(R^{g})₂, —O—R^{g}, a heterocycle, and a carbocyclic ring that is optionally substituted with one or more groups independently selected from halogen and C₁₋₆ alkyl;
each R^{g} in formula (14) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{g}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{h} in formula (14) is independently selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkoxy, and C₁₋₃ alkyl that is optionally substituted with one or more groups independently selected from halogen;
R¹ in formula (15) is selected from C₆₋₂₀ aryl, C₁₋₂₀ heteroaryl, —(C₆₋₂₀ aryl)-(C₁₋₂₀ heteroaryl), and —(C₁₋₂₀ heteroaryl)-(C₁₋₂₀ heteroaryl), wherein each of C₆₋₂₀ aryl, C₁₋₂₀ heteroaryl, —(C₆₋₂₀ aryl)-(C₁₋₂₀ heteroaryl), and (C₁₋₂₀ heteroaryl)-(C₁₋₂₀ heteroaryl) is independently optionally substituted with one or more substituents independently selected from R^{c}, oxo, fluorine, chlorine, bromine, iodine, —NO₂, —N(R³)₂, —CN, —C(O)—N(R³)₂, —S(O)—N(R¹)₂, —S(O)₂—N(R¹)₂, —O—R^{a}, —S—R^{a}, —O—C(O)—R^{a}, —O—C(O)—O—R^{a}, —C(O)—R^{a}, —C(O)—O—R^{a}, —S(O)—R^{a}, —S(O)₂—R^{a}, —O—C(O)—N(R^{a})₂, —N(R^{a})—C(O)—OR^{a}, —N(R^{a})—C(O)—N(R^{a})₂, —N(R^{a})—C(O)—R^{a}, —N(R^{a})—S(O)—R^{a}, —N(R^{a})—S(O)₂—R^{a}, —N(R^{a})—S(O)—N(R^{a})₂, and —N(R^{a})—S(O)₂—N(R^{a})₂;
R² in formula (15) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle in R² is optionally substituted with one or more R^{b};
R³ in formula (15) is C₁₋₄ alkyl, C₂₋₄ alkenyl, C₂₋₄ alkynyl, a 3- to 5-membered carbocyclic ring, a 3- to 5-membered heterocycle, —C(O)—N(R^{e})₂, —S(O)—N(R^{e})₂, —S(O)₂—N(R^{e})₂, —C(O)—R^{e}, —C(O)—OR^{e}, —S(O)—R^{e}, or —S(O)₂—R^{e}, wherein any of C₁₋₄ alkyl, C₂₋₄ alkenyl, C₂₋₄ alkynyl, 3- to 5-membered carbocyclic ring, and 3- to 5-membered heterocycle is optionally substituted with one or more substituents independently selected from fluorine, chlorine, bromine, iodine, a 3- to 5-membered carbocyclic ring, —C(O)—N(R^{e})₂, —S(O)—N(R^{e})₂, —S(O)₂—N(R^{e})₂, —O—R^{e}, —S—R^{e}, —O—C(O)—R^{e}, —O—C(O)—O—R^{e}, —C(O)—R^{e}, —C(O)—O—R^{e}, —S(O)—R^{e}, —S(O)₂—R^{e}, —O—C(O)—N(R^{e})₂, —N(R^{e})—C(O)—OR^{e}, —N(R^{e})—C(O)—N(R^{e})₂, —N(R^{e})—C(O)—R^{e}, —N(R^{e})—S(O)—R^{e}, —N(R^{e})—S(O)₂—R^{e}, —N(R^{e})—S(O)—N(R^{e})₂, and —N(R^{e})—S(O)₂—N(R^{e})₂;
each R^{a} in formula (15) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{a}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{b} in formula (15) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —O—C(O)—O—R^{c}, —C(O)—R^{c}, —C(O)—O—R^{c}, —S(O)—R^{c}, —S(O)₂—R^{c}, —O—C(O)—N(R^{c})₂, —N(R^{c})—C(O)—OR^{c}, —N(R^{c})—C(O)—N(R^{c})₂, —N(R^{c})—C(O)—R^{c}, —N(R^{c})—S(O)—R^{c}, —N(R^{c})—S(O)₂—R^{c}, —N(R^{c})—S(O)—N(R^{c})₂, and —N(R^{c})—S(O)₂—N(R^{c})₂, wherein any of C₁₋₆ alkyl, C₂₋₆ alkenyl, C₂₋₆ alkynyl, carbocyclic ring, heterocycle, aryl, and heteroaryl is optionally substituted with one or more groups independently selected from oxo, halogen, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —C(O)—R^{c}, —C(O)—O—R^{c}, —S(O)—R^{c}, —S(O)₂—R^{c}, —C(O)—N(R^{c})₂, —N(R^{c})—C(O)—R^{c}, —N(R^{c})—S(O)—R^{c}, —N(R^{c})—S(O)₂—R^{c}, and C₁₋₆ alkyl that is optionally substituted with one or more groups independently selected from oxo and halogen;
each R^{c} in formula (15) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —O—C(O)—R^{d}, —C(O)—R^{d}, —C(O)—O—R^{d}, —S(O)—R^{d}, —S(O)₂—R^{d}, —C(O)—N(R^{d})₂, —N(R^{d})—C(O)—R^{d}, —N(R^{d})—S(O)—R^{d}, —N(R^{d})—S(O)₂—R^{d}, and C₁₋₆ alkyl, and the carbocyclic ring and C₁₋₆ alkyl are optionally substituted with one or more groups independently selected from oxo, halogen, C₁₋₆ alkyl, cyano, —N(R^{d})₂, —O—R^{d}, a heterocycle, and a carbocyclic ring that is optionally substituted with one or more groups independently selected from halogen and C₁₋₆ alkyl;
each of R^{d} in formula (15) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{d}, together with the nitrogen to which they are attached, form a heterocycle optionally substituted with one or more groups independently selected from oxo, halogen, and C₁₋₃ alkyl that is optionally substituted with one or more groups independently selected from oxo and halogen; and
each R^{e} in formula (15) is selectively selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkoxy, and C₁₋₃ alkyl that is optionally substituted with one or more groups independently selected from halogen,
with the proviso that if R² is carboxymethyl or 2-carboxyethyl, R¹ is not unsubstituted phenyl,
   or a prodrug thereof or a pharmaceutically acceptable salt thereof.

### [Item 66]

The pharmaceutical composition of item 42, wherein the low molecular weight compound is a compound represented by the following (Table 12)

TABLE 33-1

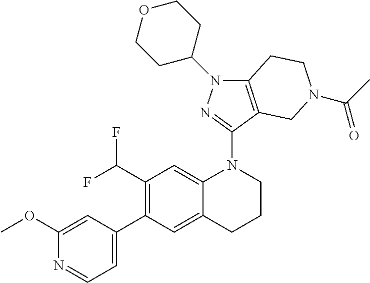 [SMILES: COc1cc(-c2cc3c(cc2C(F)F)N(c2nn(C4CCOCC4)c4c2CN(C(C)=O)CC4)CCC3)ccn1]

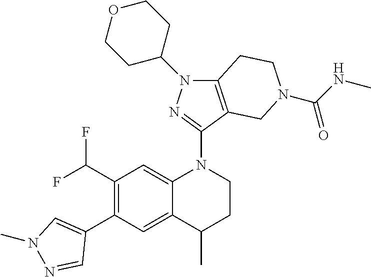

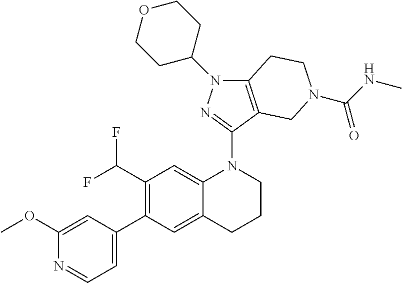

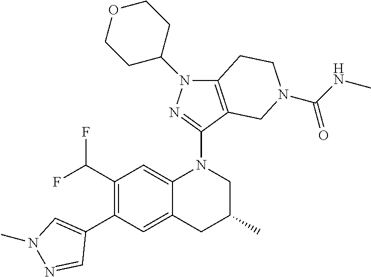

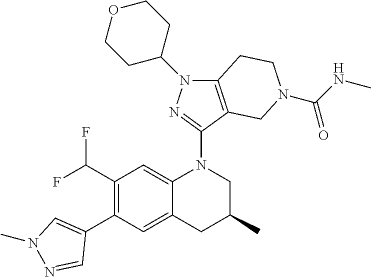

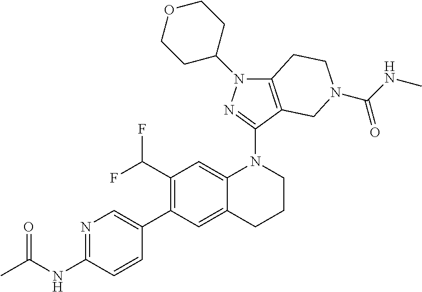 [SMILES: CNC(=O)N1CCc2c(c(N3CCCc4cc(-c5ccc(NC(C)=O)nc5)c(C(F)F)cc43)nn2C2CCOCC2)C1]

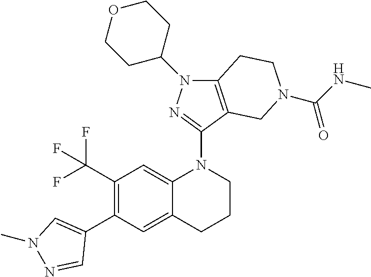

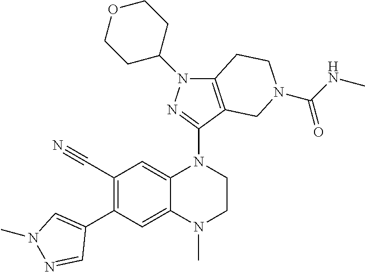

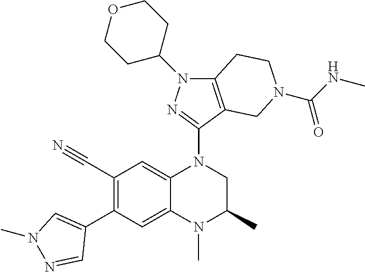

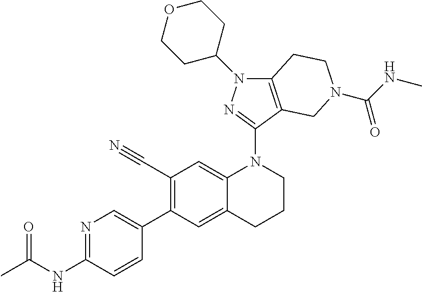 [SMILES: CNC(=O)N1CCc2c(c(N3CCCc4cc(-c5ccc(NC(C)=O)nc5)c(C#N)cc43)nn2C2CCOCC2)C1]

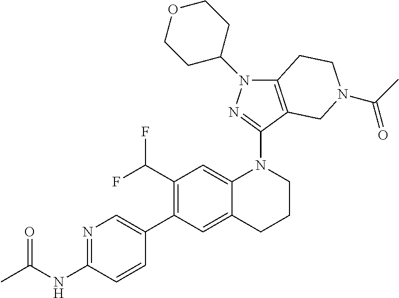 [SMILES: CC(=O)Nc1ccc(-c2cc3c(cc2C(F)F)N(c2nn(C4CCOCC4)c4c2CN(C(C)=O)CC4)CCC3)cn1]

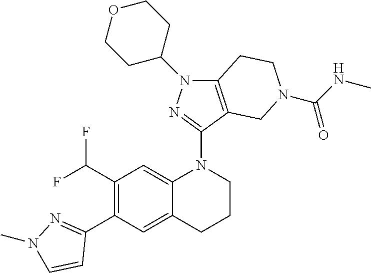

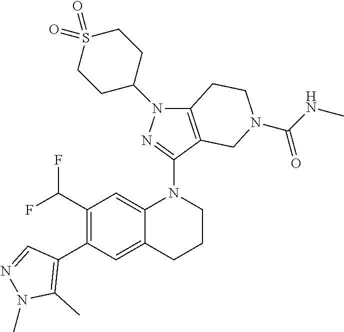 [SMILES: CNC(=O)N1CCc2c(c(N3CCCc4cc(-c5cnn(C)c5C)c(C(F)F)cc43)nn2C2CCS(=O)(=O)CC2)C1]

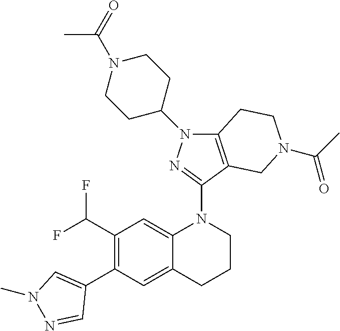 [SMILES: CC(=O)N1CCC(n2nc(N3CCCc4cc(-c5cnn(C)c5)c(C(F)F)cc43)c3c2CCN(C(C)=O)C3)CC1]

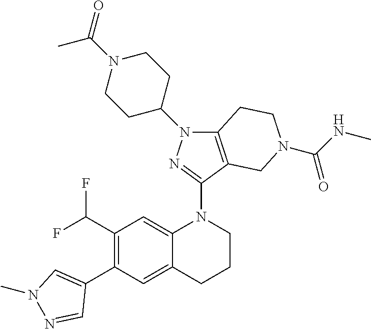

TABLE 33-2

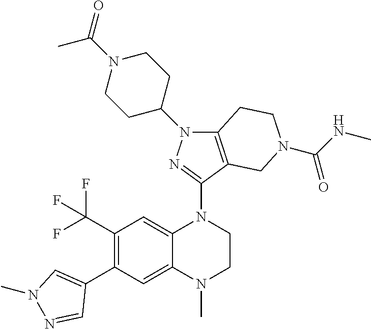

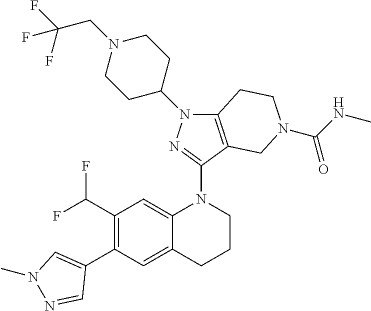

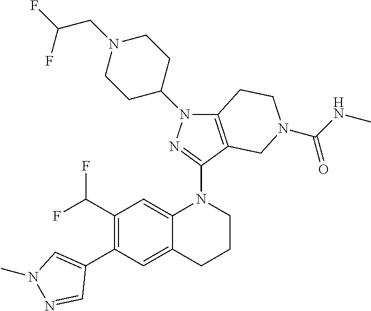

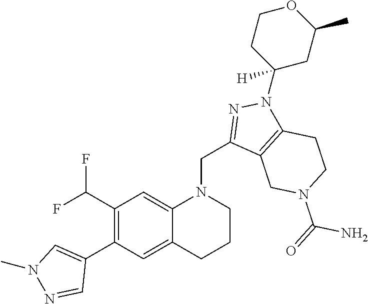

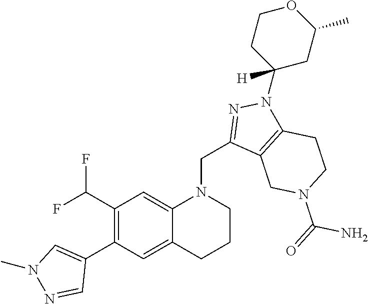

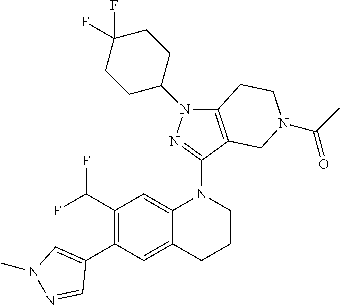 [SMILES: CC(=O)N1CCc2c(c(N3CCCc4cc(-c5cnn(C)c5)c(C(F)F)cc43)nn2C2CCC(F)(F)CC2)C1]

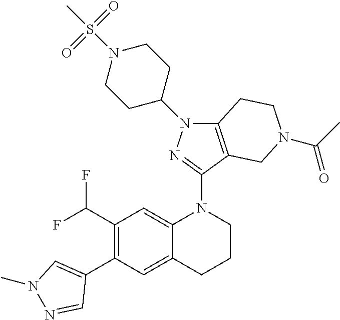 [SMILES: CC(=O)N1CCc2c(c(N3CCCc4cc(-c5cnn(C)c5)c(C(F)F)cc43)nn2C2CCN(S(C)(=O)=O)CC2)C1]

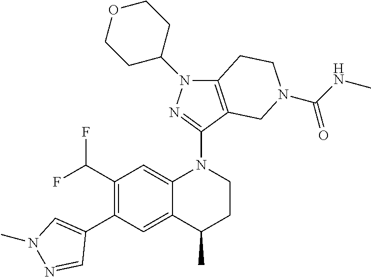

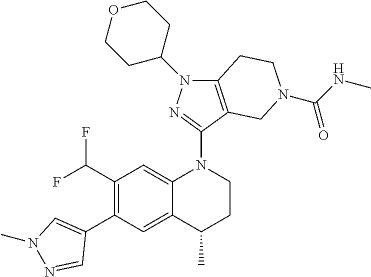

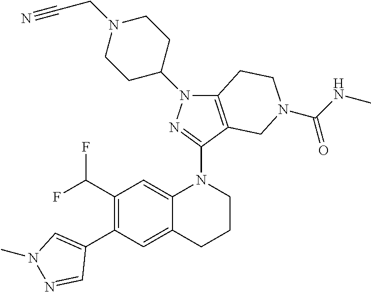

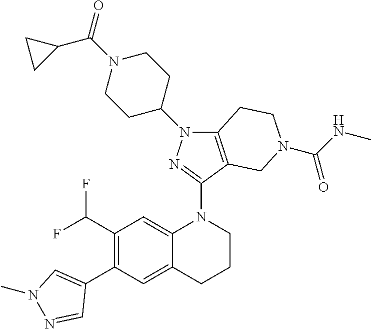

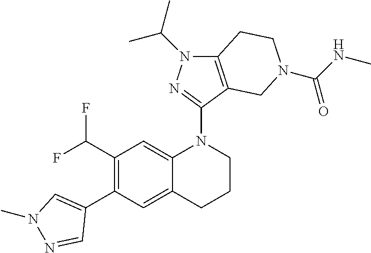

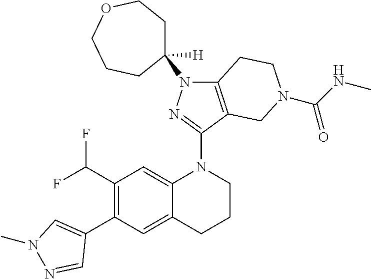

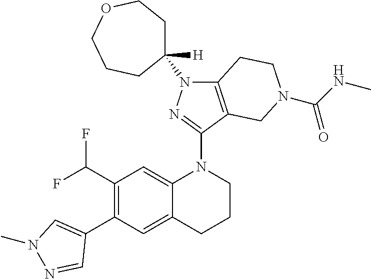

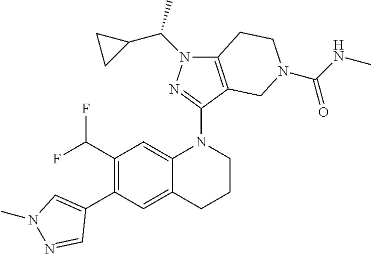

TABLE 33-3

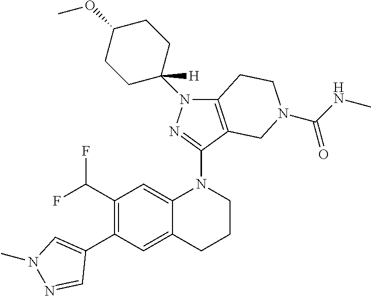

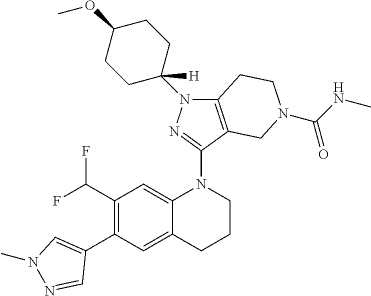

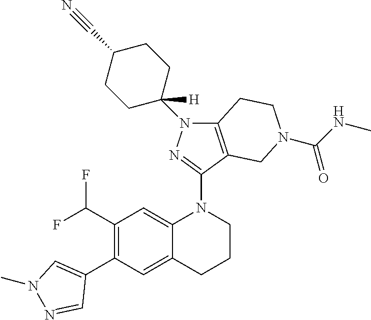

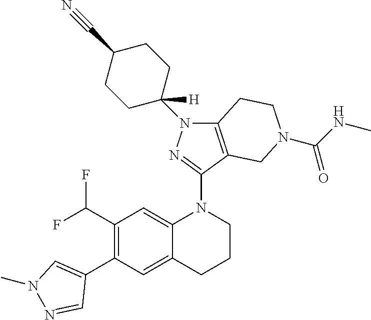

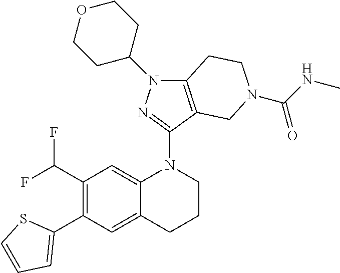 [SMILES: CNC(=O)N1CCc2c(c(N3CCCc4cc(-c5cccs5)c(C(F)F)cc43)nn2C2CCOCC2)C1]

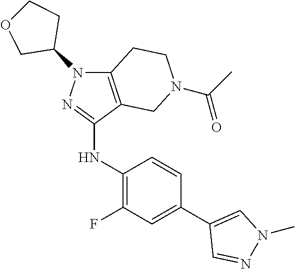 [SMILES: CC(=O)N1CCc2c(c(Nc3ccc(-c4cnn(C)c4)cc3F)nn2[C@@H]2CCOC2)C1]

or a pharmaceutically acceptable salt thereof.

### [Item 67]

The pharmaceutical composition of item 42, wherein the low molecular weight compound is a compound represented by formula (14)

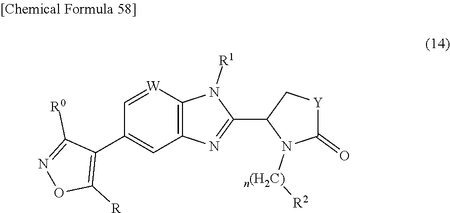

wherein
R⁰ and R are the same or different, each a hydrogen atom, or C₁₋₆ alkyl, which is unsubstituted or substituted with OH, —OC(O)R′, or OR′ (wherein R′ is unsubstituted C₁₋₆ alkyl);
W is N or CH;
R¹ is an unsubstituted or substituted group, which is C-linked 4- to 6-membered heterocyclyl, C₃₋₆ cycloalkyl, or C₁₋₆ alkyl that is unsubstituted or substituted with C₆₋₁₀ aryl, 5- to 12-membered N-containing heteroaryl, C₃₋₆ cycloalkyl, OH, —OC(O)R′, or OR′ (wherein R′ is as defined above, or a group represented by:

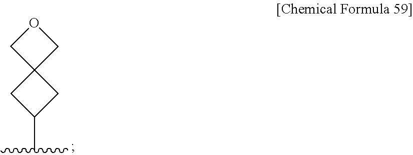 [SMILES: *C1CC2(COC2)C1]

Y is —CH₂—, —CH₂CH₂—, or CH₂CH₂CH₂—;
n is 0 or 1; and
R² is a group selected from C₆₋₁₀ aryl, 5- to 12-membered N-containing heteroaryl, C₃₋₆ cycloalkyl, and C₅₋₆ cycloalkenyl, which are unsubstituted or substituted, and the C₆₋₁₀ aryl may be fused to a 5- or 6-membered heterocycle,
   or a prodrug thereof or a pharmaceutically acceptable salt thereof.

### [Item 68]

The pharmaceutical composition of item 42, wherein the low molecular weight compound is a compound represented by

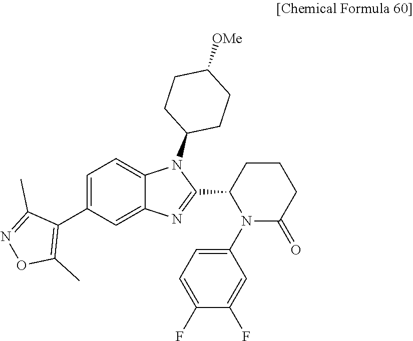 [SMILES: CO[C@H]1CC[C@H](n2c([C@@H]3CCCC(=O)N3c3ccc(F)c(F)c3)nc3cc(-c4c(C)noc4C)ccc32)CC1]

or a pharmaceutically acceptable salt thereof.

### [Item 69]

The pharmaceutical composition of item 42, wherein the compound is a compound represented by formula (15)

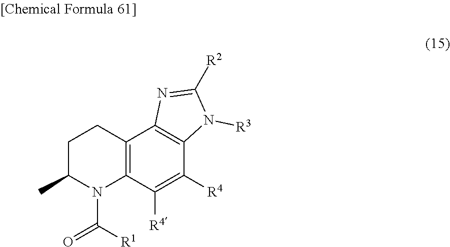

wherein
R¹ is —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, or OR⁵;
R² is hydrogen, —C₁₋₆ alkyl, —C₂₋₆ alkenyl, C₂₋₆ alkynyl, —C₃₋₈ cycloalkyl, —C₄₋₈ cycloalkenyl, heterocyclyl, heteroaryl, or aryl, wherein each of alkyl, alkenyl, alkynyl, cycloalkyl, cycloalkenyl, heterocyclyl, heteroaryl, and aryl is optionally substituted with one or more R⁶, and a —C₁₋₆ alkyl group has one or more methylene units optionally substituted with —NR⁶—, —O—, or —S—;
R³ is hydrogen, —C₁₋₆ alkyl, —C₂₋₆ alkenyl, —C₂₋₆ alkynyl, —C₃₋₈ cycloalkyl, —C₄₋₈ cycloalkenyl, spirocycloalkyl, spiroheterocyclyl, heterocyclyl, heteroaryl, or aryl, and each of alkyl, alkenyl, alkynyl, cycloalkyl, cycloalkenyl, spirocycloalkyl, spiroheterocyclyl, heterocyclyl, heteroaryl, and aryl is optionally substituted with one or more R⁷;
R⁴ and R^{4′} are each independently —H, halogen, —OH, —CN, or NH₂;
R⁵ is —C₁₋₆ alkyl, —C₃₋₈ cycloalkyl, heterocyclyl, aryl, or heteroaryl;
R⁶ and R⁷ are each independently, for each instance, hydrogen, —C₁₋₆ alkyl, —C₃₋₈ cycloalkyl, —C₄₋₈ cycloalkenyl, heterocyclyl, aryl, spirocycloalkyl, spiroheterocyclyl, heteroaryl, —OH, halogen, oxo, —CN, —SR⁸, —OR⁸, —(CH₂)ₙ—OR⁸, —NHR⁸, —NR⁸R⁹, —S(O)₂NR⁸R⁹, —S(O)₂R^{8′}, —C(O)R^{8′}, —C(O)OR⁸, —C(O)NR⁸R⁹, —NR⁸C(O)R^{9′}, —NR⁸S(O)₂R^{9′}, —S(O)R^{8′}, —S(O)NR⁸R⁹, or NR⁸S(O)R^{9′}, wherein each of alkyl, cycloalkyl, heterocyclyl, spirocycloalkyl, spiroheterocyclyl, heteroaryl, and aryl is optionally substituted with one or more R¹⁰;
wherein any two R⁶ or any two R⁷, when on non-adjacent atoms, can attach and form bridged cycloalkyl or heterocyclyl, wherein any two R⁶ or any two R⁷, when on adjacent atoms, can attach and form cycloalkyl, heterocyclyl, aryl, or heteroaryl;
R⁸ and R⁹ are each independently, for each instance, —H, —C₁₋₆ alkyl, —C₂₋₆ alkenyl, —C₂₋₆ alkynyl, —C₃₋₈ cycloalkyl, —C₄₋₈ cycloalkenyl, heterocyclyl, aryl, or heteroaryl, wherein each of alkyl, alkenyl, alkynyl, cycloalkyl, cycloalkenyl, heterocyclyl, aryl, and heteroaryl is optionally substituted with one or more R¹⁰ or R¹¹; or
R⁸ and R⁹, attached to an atom to which they are both attached, may form —C₃₋₈ cycloalkyl, —C₄₋₈ cycloalkenyl, spirocycloalkyl, spiroheterocyclyl, heterocyclyl, heteroaryl, or aryl, and the formed —C₃₋₈ cycloalkyl, —C₄₋₈ cycloalkenyl, spirocycloalkyl, spiroheterocyclyl, heterocyclyl, heteroaryl, or aryl is optionally substituted with one or more R¹⁰ or R¹¹;
R^{8′} and R^{9′} are each independently, for each instance, —C₁₋₆ alkyl, —C₂₋₆ alkenyl, —C₂₋₆ alkynyl, —C₃₋₈ cycloalkyl, —C₄₋₈ cycloalkenyl, heterocyclyl, aryl, or heteroaryl, wherein each of alkyl, alkenyl, alkynyl, cycloalkyl, cycloalkenyl, heterocyclyl, aryl, and heteroaryl is optionally substituted with one or more R¹⁰ or R¹¹; or
R⁸ and R^{9′}, attached to an atom to which they are both attached, may form —C₃₋₈ cycloalkyl, —C₄₋₈ cycloalkenyl, spirocycloalkyl, spiroheterocyclyl, heterocyclyl, heteroaryl, or aryl, and —C₃₋₈ cycloalkyl, —C₄₋₈ cycloalkenyl, spirocycloalkyl, spiroheterocyclyl, heterocyclyl, heteroaryl, or aryl is optionally substituted with one or more R¹⁰ or R¹¹;
R¹⁰ and R¹¹ are each independently, for each instance, hydrogen, —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, —OH, halogen, oxo, —NO₂, —CN, —NH₂, —OC₁₋₆ alkyl, —NHC₁₋₆ alkyl, —N(C₁₋₆ alkyl)₂, —S(O)₂NH(C₁₋₆ alkyl), —S(O)₂N(C₁₋₆ alkyl)₂, —S(O)₂C₁₋₆ alkyl, —C(O) C₁₋₆ alkyl, —C(O)NH₂, —C(O)NH(C₁₋₆ alkyl), —C(O)N(C₁₋₆ alkyl)₂, —C(O) OC₁₋₆ alkyl, —N(C₁₋₆ alkyl) SO₂C₁₋₆ alkyl, —S(O)(C₁₋₆ alkyl), —S(O)N(C₁₋₆ alkyl)₂, or N(C₁₋₆ alkyl)S(O)(C₁₋₆ alkyl), wherein each of alkyl, alkenyl, alkynyl, cycloalkyl, cycloalkenyl, heterocyclyl, heteroaryl, and aryl is optionally substituted with one or more R¹²;
wherein any two R¹⁰ or any two R¹¹, when on non-adjacent atoms, can attach and form bridged cycloalkyl or heterocyclyl;
wherein any two R¹⁰ or any two R¹¹, when on adjacent atoms, can attach and form cycloalkyl, heterocyclyl, aryl, or heteroaryl;
R¹² is each independently, for each instance, —H, —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, —OH, halogen, oxo, —NO₂, —CN, —NH₂, —OC₁₋₆ alkyl, —NHC₁₋₆ alkyl, —N(C₁₋₆ alkyl)₂, —S(O)₂NH(C₁₋₆ alkyl), —S(O)₂N(C₁₋₆ alkyl), —S(O) C₁₋₆ alkyl, —C(O) C₁₋₆ alkyl, —C(O)NH₂, —C(O)NH(C₁₋₆ alkyl), —C(O)N(C₁₋₆ alkyl), —C(O) OC₁₋₆ alkyl, —N(C₁₋₆ alkyl)SO₂C₁₋₆ alkyl, —S(O)(C₁₋₆ alkyl), —S(O)N(C₁₋₆ alkyl)₂, or N(C₁₋₆ alkyl)S(O)(C₁₋₆ alkyl), and
n is an integer from 1 to 4,
   or a prodrug thereof or a pharmaceutically acceptable salt thereof.

### [Item 70]

The pharmaceutical composition of item 42, wherein the low molecular weight compound is a compound represented by the following (Table 13)

TABLE 34

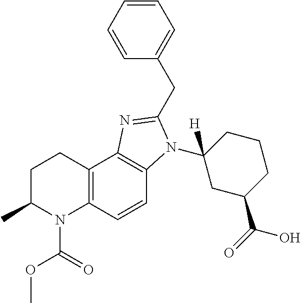 [SMILES: COC(=O)N1c2ccc3c(nc(Cc4ccccc4)n3[C@@H]3CCC[C@@H](C(=O)O)C3)c2CC[C@@H]1C]

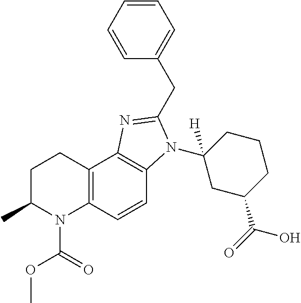 [SMILES: COC(=O)N1c2ccc3c(nc(Cc4ccccc4)n3[C@H]3CCC[C@H](C(=O)O)C3)c2CC[C@@H]1C]

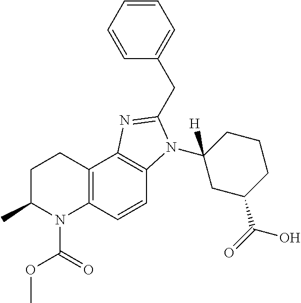 [SMILES: COC(=O)N1c2ccc3c(nc(Cc4ccccc4)n3[C@@H]3CCC[C@H](C(=O)O)C3)c2CC[C@@H]1C]

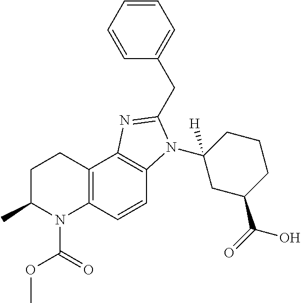 [SMILES: COC(=O)N1c2ccc3c(nc(Cc4ccccc4)n3[C@H]3CCC[C@@H](C(=O)O)C3)c2CC[C@@H]1C]

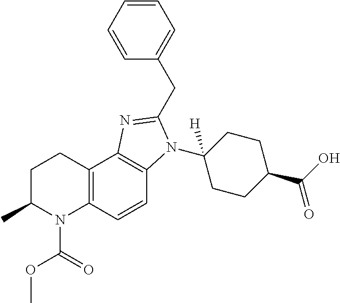 [SMILES: COC(=O)N1c2ccc3c(nc(Cc4ccccc4)n3[C@H]3CC[C@@H](C(=O)O)CC3)c2CC[C@@H]1C]

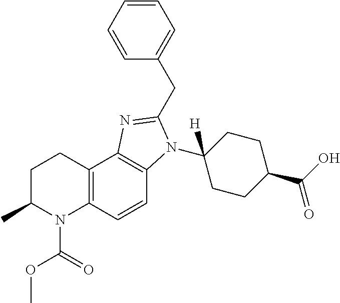 [SMILES: COC(=O)N1c2ccc3c(nc(Cc4ccccc4)n3[C@H]3CC[C@H](C(=O)O)CC3)c2CC[C@@H]1C]

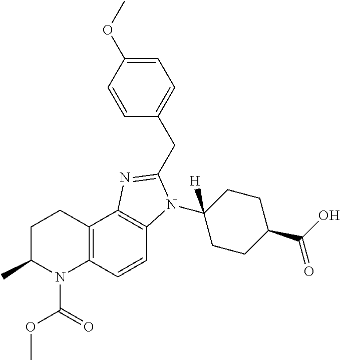 [SMILES: COC(=O)N1c2ccc3c(nc(Cc4ccc(OC)cc4)n3[C@H]3CC[C@H](C(=O)O)CC3)c2CC[C@@H]1C]

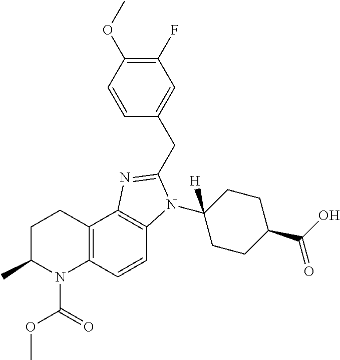 [SMILES: COC(=O)N1c2ccc3c(nc(Cc4ccc(OC)c(F)c4)n3[C@H]3CC[C@H](C(=O)O)CC3)c2CC[C@@H]1C]

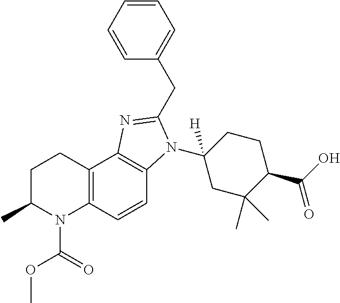 [SMILES: COC(=O)N1c2ccc3c(nc(Cc4ccccc4)n3[C@H]3CC[C@@H](C(=O)O)C(C)(C)C3)c2CC[C@@H]1C]

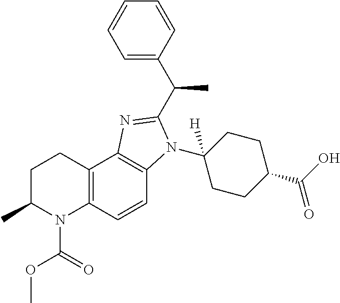 [SMILES: COC(=O)N1c2ccc3c(nc([C@H](C)c4ccccc4)n3[C@H]3CC[C@H](C(=O)O)CC3)c2CC[C@@H]1C]

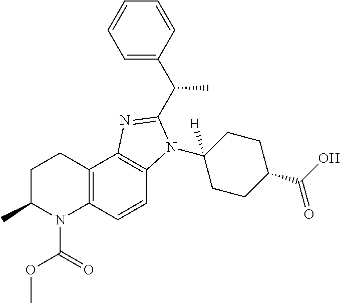 [SMILES: COC(=O)N1c2ccc3c(nc([C@@H](C)c4ccccc4)n3[C@H]3CC[C@H](C(=O)O)CC3)c2CC[C@@H]1C]

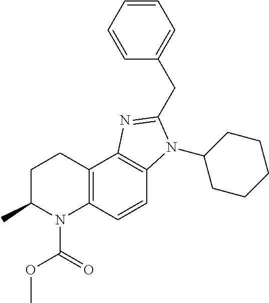 [SMILES: COC(=O)N1c2ccc3c(nc(Cc4ccccc4)n3C3CCCCC3)c2CC[C@@H]1C]

or a pharmaceutically acceptable salt thereof.

### [Item 71]

The pharmaceutical composition of item 42, wherein the low molecular weight compound is a compound represented by formula (16)

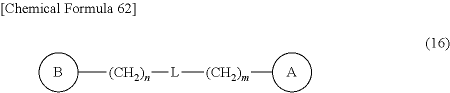

wherein
ring B is a group having the following structure;

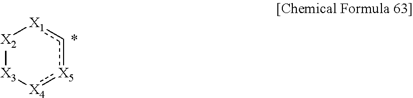

one of ring atoms X₂ and X₃ is N(R^{X1}), and the other one of the ring atoms X₂ and X₃ is C(═O);
ring atom X₁ is selected from N(R^{X1}), C(R^{X2}), and C(═O), and ring atoms X₄ and X₅ are each independently selected from N(R^{X1}), C(R^{X3}), and C(═O); wherein at least one of the ring atoms X₁, X₄, and X₅ is different from N(R^{X1}) and C(═O); and wherein if X₃ and X₅ are C(═O), X₄ is N(R^{X1}), and X₁ is C(R^{X2}), X₂ is N(H);
each

[symbol]

[Chemical Formula 64]

is independently a single bond or a double bond; wherein at least one of any two adjacent bonds

[symbol]

[Chemical Formula 65]

is a single bond;
each R^{X1} is independently selected from hydrogen, C₁₋₅ alkyl, —CO(C₁₋₅ alkyl), —(C₀₋₃ alkylene)-aryl, and heteroaryl, wherein aryl in the —(C₀₋₃ alkylene)-aryl and the heteroaryl are each optionally substituted with one or more groups R^{X11};
R^{X2} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, (C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
two groups R^{X3} are linked to each other and, together with the ring carbon to which they are attached, form a 5- or 6-membered cyclyl group optionally substituted with one or more groups R^{X31}, or two groups R^{X3} are each independently selected from hydrogen, C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl) (C₁₋₅ alkyl), halogen, C₁₋₅ haloalkyl, —O—(C₁₋₅ haloalkyl), —CF₃, —CN, —NO₂, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl) (C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl) (C₁₋₅ alkyl), —NH—SO₂—(C₁₋₅ alkyl), and —N(C₁₋₅ alkyl)-SO₂—(C₁₋₅ alkyl);
each R^{X11} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl)-(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
each R^{X31} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—N(C₁₋₅ alkyl), —(C₀₋₃ alkylene)-SO₂—N(C₁₋₅ alkyl) (C₁₋₅ alkyl), —(C₀₋₃ alkylene)-NH—SO—(C₁₋₅ alkyl), and —(C₀₋₃ alkylene)-N(C₁₋₅ alkyl)-SO₂—(C₁₋₅ alkyl);
if ring B is attached to the remaining portion of a compound of formula (19) via a ring carbon atom marked with an asterisk (*), or X₄ and X₅ are each C(R^{X3}) and two groups R^{X3} are linked to each other and, together with the ring carbon atom to which they are attach, form a 5- or 6-membered cyclyl group optionally substituted with one or more groups R^{X31}, ring B may be attached to the remaining portion of a compound of formula (19) via any carbocyclic ring atom of the 5- or 6-membered cyclyl group;
ring A is aryl or heteroaryl, wherein the aryl and the heteroaryl are optionally substituted with one or more groups R^{A}, wherein the heteroaryl is selected from 1,4-benzodioxanyl, benzoxanyl, 1,3-benzodioxolanyl, benzoxolanyl, and 1,5-benzodioxepanyl;
each R^{A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl), —(C₀₋₃ alkylene)-NH—SO₂—(C₁₋₅ alkyl), —(C₀₋₃ alkylene)-N(C₁₋₅ alkyl)-SO—(C₁₋₅ alkyl), —(C₀₋₃ alkylene)-cycloalkyl, —(C₀₋₃ alkylene)-O-cycloalkyl, —(C₀₋₃ alkylene)-O(C₁₋₅ alkylene)-cycloalkyl, —(C₀₋₃ alkylene)-heterocycloalkyl, —(C₀₋₃ alkylene)-O-heterocycloalkyl, and —(C₀₋₃ alkylene)-O(C₁₋₅ alkylene)-heterocycloalkyl;
L is selected from —CO—N(R^{L1})—, —N(R^{L1})—CO—, —CO—O—, —O—CO—, —C(═N—R^{L2})—N(R^{L1})—, —N(R^{L1})—C(═N—R^{L2})—, —C(═S)—N(R^{L1})—, —N(R^{L1})—C(═S)—, —N(R^{L1})—CO—N(R^{L1})—, —O—CO—N(R^{L1})—, —N(R^{L1})—CO—O—, —N(R^{L1})—C(═N—R^{L2})—N(R^{L1}), —O—C(═N—R^{L2})—N(R^{L1})—, —N(R^{L1})—C(═N—R^{L2})—O—, —S—C(═N—R^{L2})—N(R^{L1})—, —N(R^{L1})—C(═N—R^{L2})—S—, —N(R^{L1})—C(═S)—N(R^{L1}), —O—C(═S)—N(R^{L1}), —N(R^{L1})—C(═S)—O—, —S—CO—N(R^{L1})—, and —N(R^{L1})—CO—S—;
each R^{L1} is independently selected from hydrogen and C₁₋₅ alkyl;
each R^{L2} is independently selected from hydrogen, C₁₋₅ alkyl, —CN, and —NO₂;
n is 0 or 1; and
m is 0 or 1,
   or a prodrug thereof or a pharmaceutically acceptable salt thereof.

### [Item 72]

The pharmaceutical composition of item 42, wherein the low molecular weight compound is a compound represented by the following (Table 14)

TABLE 35

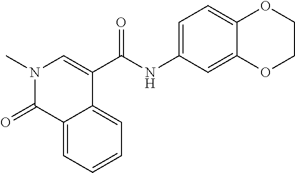 [SMILES: Cn1cc(C(=O)Nc2ccc3c(c2)OCCO3)c2ccccc2c1=O]

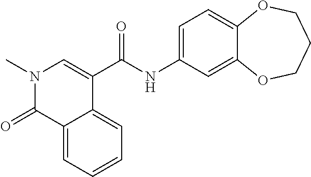 [SMILES: Cn1cc(C(=O)Nc2ccc3c(c2)OCCCO3)c2ccccc2c1=O]

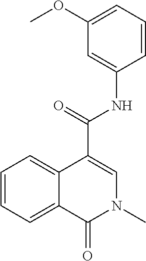 [SMILES: COc1cccc(NC(=O)c2cn(C)c(=O)c3ccccc23)c1]

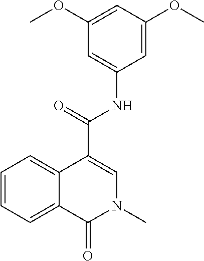 [SMILES: COc1cc(NC(=O)c2cn(C)c(=O)c3ccccc23)cc(OC)c1]

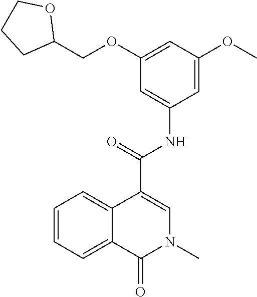 [SMILES: COc1cc(NC(=O)c2cn(C)c(=O)c3ccccc23)cc(OCC2CCCO2)c1]

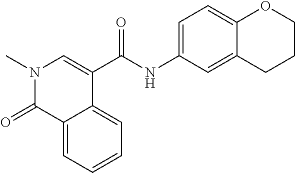 [SMILES: Cn1cc(C(=O)Nc2ccc3c(c2)CCCO3)c2ccccc2c1=O]

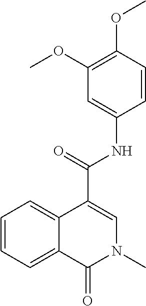 [SMILES: COc1ccc(NC(=O)c2cn(C)c(=O)c3ccccc23)cc1OC]

or a pharmaceutically acceptable salt thereof.

### [Item 73]

The pharmaceutical composition of item 42, wherein the low molecular weight compound is a compound represented by formula (17)

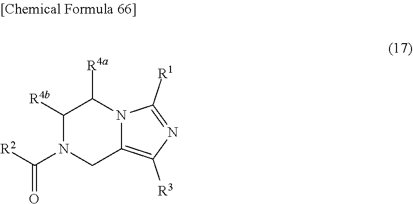

wherein
R¹ is hydrogen, or selected from alkyl, amino, alkoxy, heteroalkyl, cycloalkyl, heterocycloalkyl, halogen, haloalkyl, sulfonylalkyl, aryl, and heteroaryl, which is optionally substituted with 1, 2, or 3 group R⁵;
R² is hydrogen, or selected from alkyl, haloalkyl, amino, alkoxy, cycloalkyl, and heterocycloalkyl, which is optionally substituted with 1 or 2 group R⁶;
R³ is selected from alkyl, amino, alkoxy, heteroalkyl, cycloalkyl, heterocycloalkyl, carbonyl, sulfonyl, aryl, and heteroaryl, wherein the R³ is:
   (a) optionally substituted with 1, 2, or 3 group R⁷, and
   (b) optionally substituted with one R⁸;
R^{4a} and R^{4b} are hydrogen;
R⁵, R⁶, and R⁷ are each independently selected from alkyl, alkoxy, cyano, carboxy, halogen, haloalkyl, haloalkoxyl, hydroxy, and oxo;
R⁸ is selected from aryl, heteroaryl, and heterocycloalkyl, wherein the R⁸ is optionally substituted with 1, 2, or 3 group R¹⁰; and
R¹⁰ is each independently selected from alkyl, cycloalkyl, (cycloalkyl)alkyl, heterocycloalkyl, (heterocycloalkyl)alkyl, aryl, (aryl)alkyl, (heteroaryl)alkyl, alkoxy, cyano, carboxy, halogen, haloalkyl, haloalkoxy, hydroxy, hydroxyalkyl, oxo, CONH₂, CONHCH₃, SO₂CH₃, and SO₂NH₂,
   or a prodrug thereof or a pharmaceutically acceptable salt thereof.

### [Item 74]

The pharmaceutical composition of item 42, wherein the low molecular weight compound is a compound represented by the following (Table 15)

TABLE 36

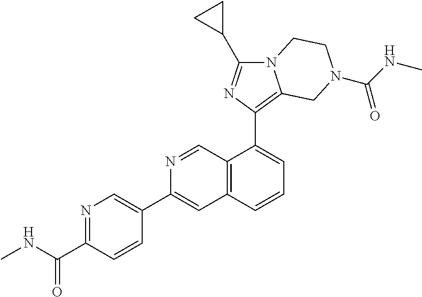

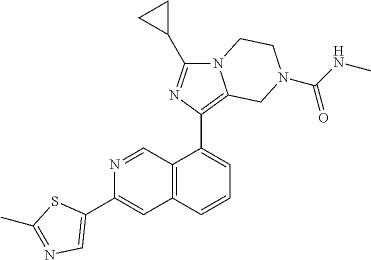

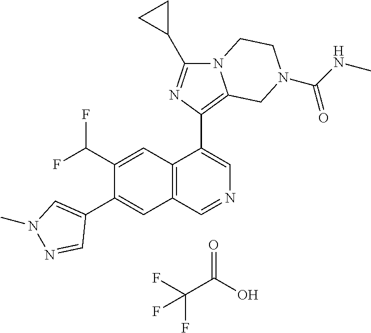

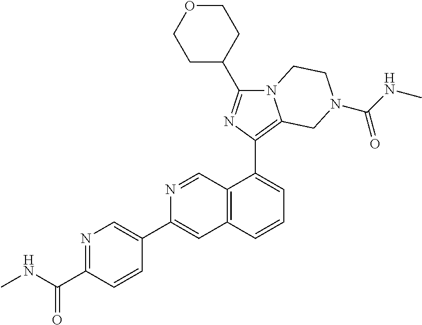

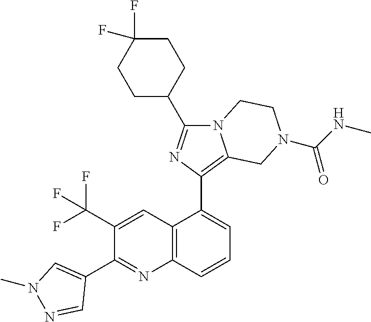

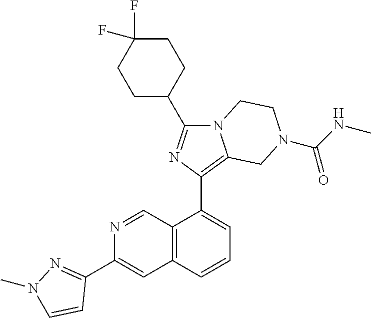

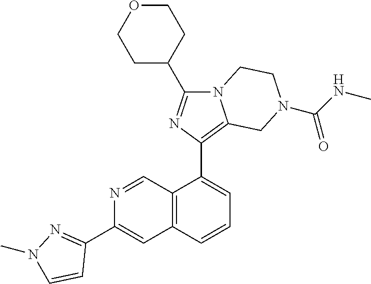

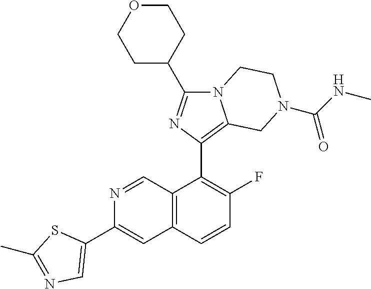

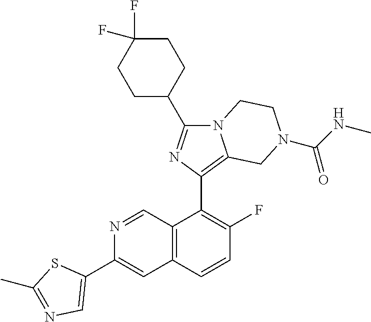

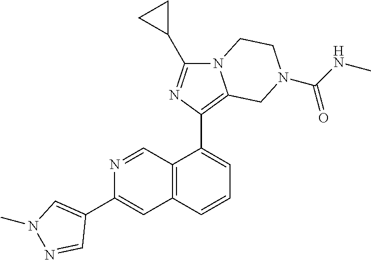

or a pharmaceutically acceptable salt thereof.

### [Item 75]

The pharmaceutical composition of item 42, wherein the low molecular weight compound is a compound represented by formula (18)

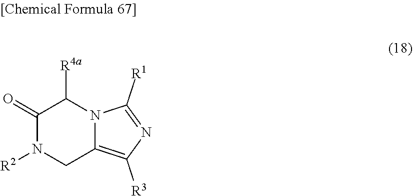

wherein
R¹ is hydrogen, or selected from alkyl, amino, alkoxy, heteroalkyl, cycloalkyl, heterocycloalkyl, halogen, haloalkyl, sulfonylalkyl, aryl, and heteroaryl, which is optionally substituted with 1, 2 or 3 group R⁵;
R² is hydrogen, or selected from alkyl, heteroalkyl, cycloalkyl, heterocycloalkyl, and haloalkyl, which is optionally substituted with 1, 2, or 3 group R⁶;
R³ is selected from alkyl, amino, alkoxy, heteroalkyl, cycloalkyl, heterocycloalkyl, alkylcarbonyl, alkylsulfonyl, arylcarbonyl, arylsulfonyl, aryl, and heteroaryl, wherein the R³ is:
   (a) optionally substituted with 1, 2, or 3 group R⁷, and
   (b) optionally substituted with one R⁸;
R^{4a} is selected from hydrogen, halogen, alkyl, heteroalkyl, cycloalkyl, and heterocycloalkyl, wherein the R^{4a} is optionally substituted with 1, 2 or 3 group R⁹;
R⁵ is each independently selected from alkyl, alkoxy, alkoxyalkyl, alkylcarbonyl, alkylsulfonyl, amino, aminocarbonyl, cyano, carboxy, halogen, haloalkoxy, haloalkyl, hydroxy, hydroxyalkyl, and oxo;
R⁶ and R⁷ are each independently selected from alkyl, alkoxy, cyano, carboxy, halogen, haloalkyl, hydroxy, and oxo;
R⁸ is selected from heterocycloalkyl, aryl, and heteroaryl, wherein the R⁰ is optionally substituted with 1, 2, or 3 group R¹⁰;
R⁹ is each independently selected from alkyl, alkoxy, cyano, carboxy, halogen, haloalkyl, hydroxy, and oxo; and
R¹⁰ is each independently selected from alkyl, alkoxy, cyano, carboxy, halogen, haloalkyl, and hydroxy,
   or a prodrug thereof or a pharmaceutically acceptable salt thereof.

### [Item 76]

The pharmaceutical composition of item 42, wherein the low molecular weight compound is a compound presented by the following (Table 16)

TABLE 37

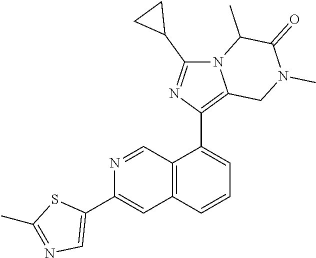

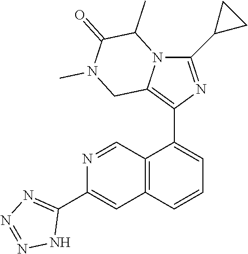 [SMILES: CC1C(=O)N(C)Cc2c(-c3cccc4cc(-c5nnn[nH]5)ncc34)nc(C3CC3)n21]

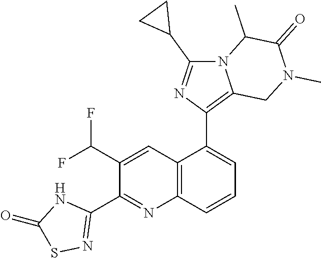 [SMILES: CC1C(=O)N(C)Cc2c(-c3cccc4nc(-c5nsc(=O)[nH]5)c(C(F)F)cc34)nc(C3CC3)n21]

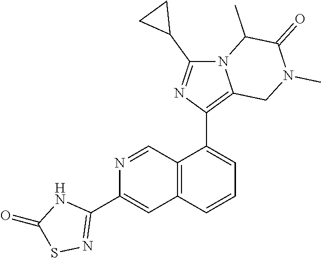 [SMILES: CC1C(=O)N(C)Cc2c(-c3cccc4cc(-c5nsc(=O)[nH]5)ncc34)nc(C3CC3)n21]

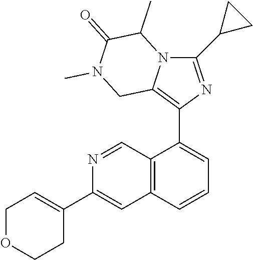 [SMILES: CC1C(=O)N(C)Cc2c(-c3cccc4cc(C5=CCOCC5)ncc34)nc(C3CC3)n21]

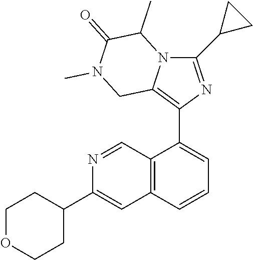 [SMILES: CC1C(=O)N(C)Cc2c(-c3cccc4cc(C5CCOCC5)ncc34)nc(C3CC3)n21]

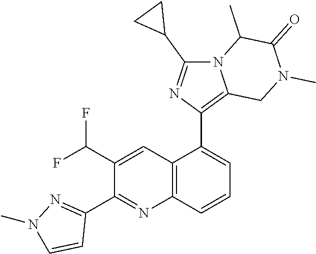

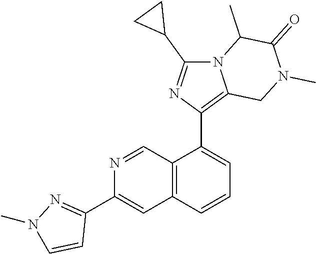

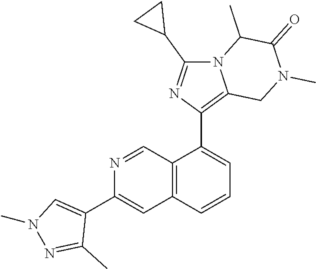

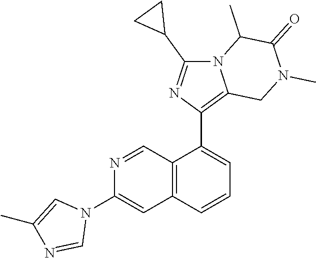

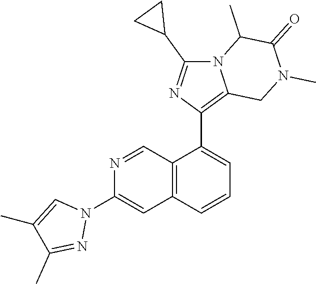

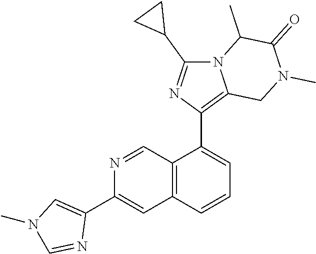

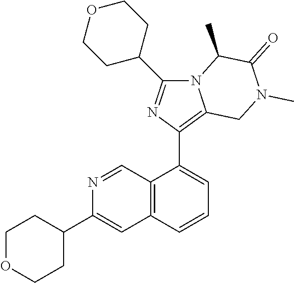 [SMILES: C[C@H]1C(=O)N(C)Cc2c(-c3cccc4cc(C5CCOCC5)ncc34)nc(C3CCOCC3)n21]

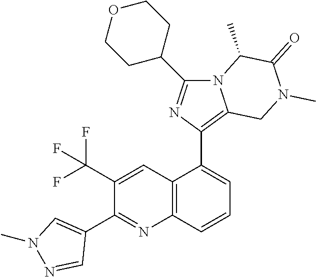

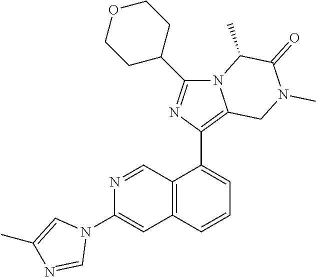

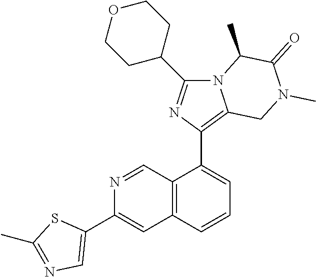

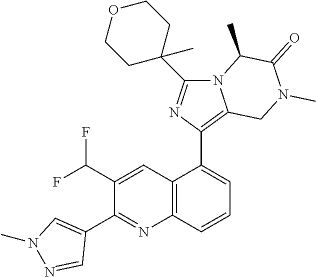

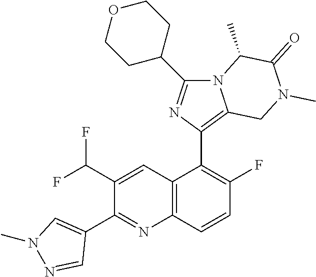

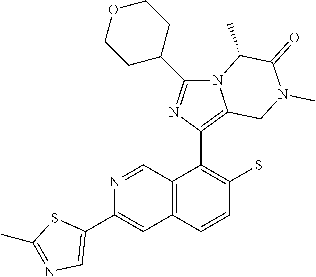

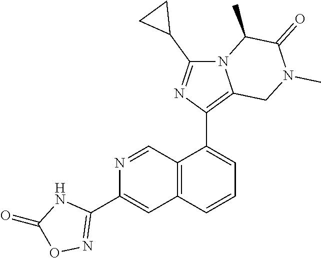 [SMILES: C[C@H]1C(=O)N(C)Cc2c(-c3cccc4cc(-c5noc(=O)[nH]5)ncc34)nc(C3CC3)n21]

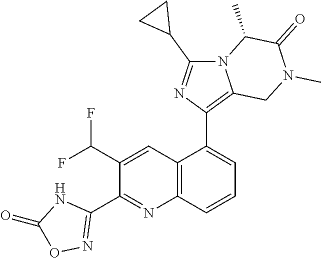 [SMILES: C[C@@H]1C(=O)N(C)Cc2c(-c3cccc4nc(-c5noc(=O)[nH]5)c(C(F)F)cc34)nc(C3CC3)n21]

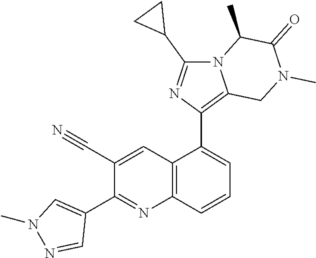

or a pharmaceutically acceptable salt thereof.

The pharmaceutical composition of item 42, wherein the low molecular weight compound is a compound represented by formula (19)

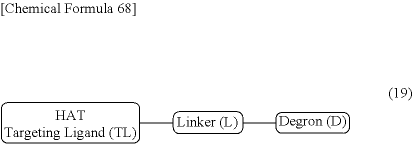

wherein
Targeting Ligand (TL) represents a structure that attaches to P300, Degron (D) represents a structure that attaches to an E3 ubiquitin ligase, and Linker (L) represents a structure that covalently attaches to Degron and Targeting Ligand,
   or a prodrug thereof or a pharmaceutically acceptable salt thereof.

### [Item 78]

The pharmaceutical composition of item 42, wherein the low molecular weight compound is a compound represented by

TABLE 38

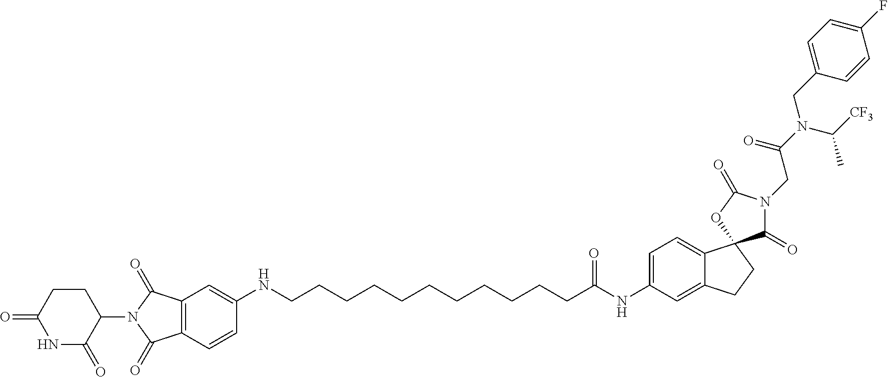 [SMILES: C[C@H](N(Cc1ccc(F)cc1)C(=O)CN1C(=O)O[C@@]2(CCc3cc(NC(=O)CCCCCCCCCCCNc4ccc5c(c4)C(=O)N(C4CCC(=O)NC4=O)C5=O)ccc32)C1=O)C(F)(F)F]

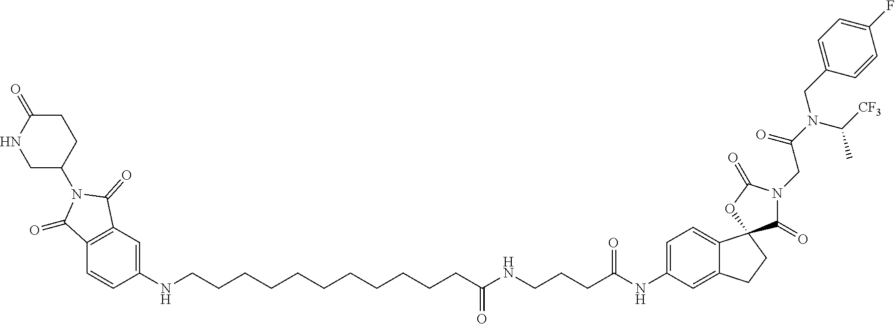 [SMILES: C[C@H](N(Cc1ccc(F)cc1)C(=O)CN1C(=O)O[C@@]2(CCc3cc(NC(=O)CCCNC(=O)CCCCCCCCCCCNc4ccc5c(c4)C(=O)N(C4CCC(=O)NC4)C5=O)ccc32)C1=O)C(F)(F)F]

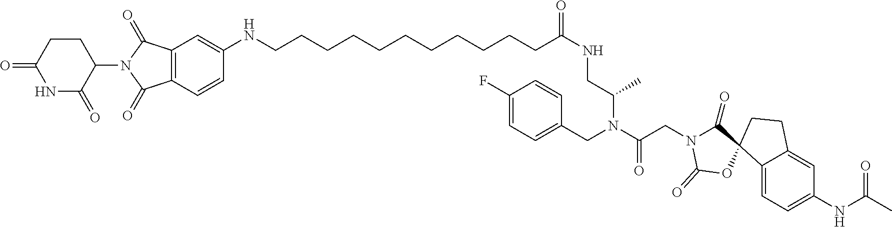 [SMILES: CC(=O)Nc1ccc2c(c1)CC[C@@]21OC(=O)N(CC(=O)N(Cc2ccc(F)cc2)[C@@H](C)CNC(=O)CCCCCCCCCCCNc2ccc3c(c2)C(=O)N(C2CCC(=O)NC2=O)C3=O)C1=O]

or a pharmaceutically acceptable salt thereof.

### [Item 79]

The pharmaceutical composition of item 42, wherein the low molecular weight compound is a compound represented by

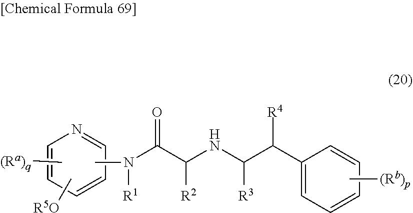

wherein
R¹, R³, and R⁴ are each independently hydrogen or C₁₋₄ alkyl;
R² is phenyl or 5- to 6-membered heteroaryl, each optionally substituted with 1 to 3 R^{c};
R⁵ is C₁₋₆ alkyl substituted with 4- to 6-membered heterocyclyl or 5- to 6-membered heteroaryl (wherein the heterocyclyl or heteroaryl is optionally substituted with 1 to 3 R^{d}), 4- to 6-membered heterocyclyl (wherein the heterocyclyl is optionally substituted with 1 to 3 R^{d}), or 5- to 6-membered heteroaryl (wherein the heteroaryl is optionally substituted with 1 to 3 R^{d});
R^{a}, R^{b}, R^{c}, and R^{d} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{c}, —C(O)R^{f}, —C(O)OR, —C₁₋₆ alkyl C(O)OR^{e}, —C(O)N(R^{e})₂, —C(O)NR^{e}C₁₋₆ alkyl OR^{e}, —OC₁₋₆ alkyl N(R^{c})₂, —C₁₋₆ alkyl C(O)N(R^{c})₂, —C₁₋₆ alkyl N(R^{c})₂, —N(R^{e})₂, —C(O)NR^{e}C₁₋₆ alkyl N(R^{e})₂, —NR^{e}C₁₋₆ alkyl N(R^{e})₂, —NR^{e}C₁₋₆ alkyl OR^{e}, —SOR^{e}, —S(O)₂R^{e}, —SON(R^{e})₂, —SO₂N(R^{e})₂, —O(C₃₋₆) cycloalkyl, —O—C₁₋₄ alkyl-aryl, —C₁₋₆ alkyl(C₃₋₆) cycloalkyl, —C₁₋₆ alkylaryl, —C₁₋₆ alkylheteroaryl, —C₁₋₆ alkylheterocyclyl, C₃₋₆ cycloalkyl, heterocyclyl, heteroaryl, or aryl (wherein each of them, alone or attached to —O(C₃₋₆)cycloalkyl, —C₁₋₆ alkyl(C₁₋₆) cycloalkyl, —C₁₋₆ alkylaryl, —C₁₋₆ alkylheteroaryl, or —C₁₋₆ alkylheterocyclyl, is optionally substituted with 1 to 3 groups selected from halogen, C₁₋₆ alkyl, C₁₋₆ haloalkyl, C₁₋₆ alkoxy, C₁₋₆ haloalkoxy, —N(R^{e})₂, —C(O)R^{f}, and —C₁₋₆ alkyl OR^{e});
each R^{e} is hydrogen, C₁₋₄ haloalkyl, or C₁₋₄ alkyl,
each R^{f} is hydrogen, C₁₋₄ haloalkyl, C₁₋₄ alkyl, or C₃₋₄ cycloalkyl,
q is 0, 1, or 2, and
p is 0, 1, 2, or 3,
   or a prodrug thereof or a pharmaceutically acceptable salt thereof.

### [Item 80]

The pharmaceutical composition of item 42, wherein the low molecular weight compound is a compound represented by

TABLE 39

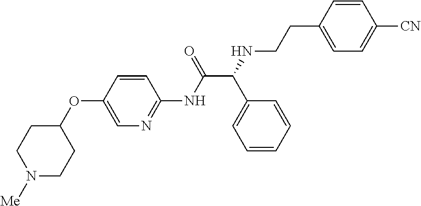 [SMILES: CN1CCC(Oc2ccc(NC(=O)[C@H](NCCc3ccc(C#N)cc3)c3ccccc3)nc2)CC1]

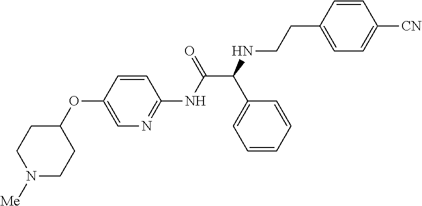 [SMILES: CN1CCC(Oc2ccc(NC(=O)[C@@H](NCCc3ccc(C#N)cc3)c3ccccc3)nc2)CC1]

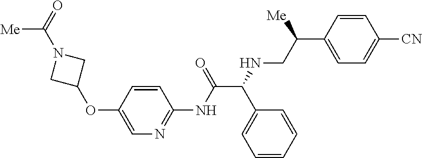 [SMILES: CC(=O)N1CC(Oc2ccc(NC(=O)[C@H](NC[C@@H](C)c3ccc(C#N)cc3)c3ccccc3)nc2)C1]

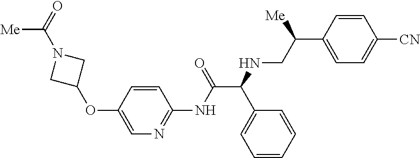 [SMILES: CC(=O)N1CC(Oc2ccc(NC(=O)[C@@H](NC[C@@H](C)c3ccc(C#N)cc3)c3ccccc3)nc2)C1]

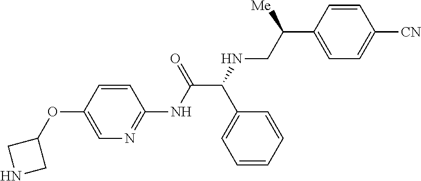 [SMILES: C[C@H](CN[C@@H](C(=O)Nc1ccc(OC2CNC2)cn1)c1ccccc1)c1ccc(C#N)cc1]

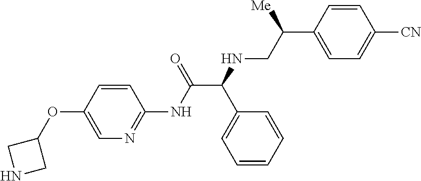 [SMILES: C[C@H](CN[C@H](C(=O)Nc1ccc(OC2CNC2)cn1)c1ccccc1)c1ccc(C#N)cc1]

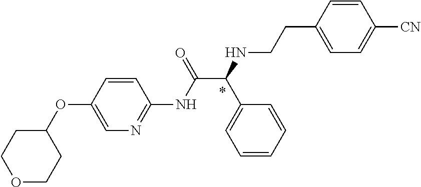 [SMILES: N#Cc1ccc(CCN[C@H](C(=O)Nc2ccc(OC3CCOCC3)cn2)c2ccccc2)cc1]

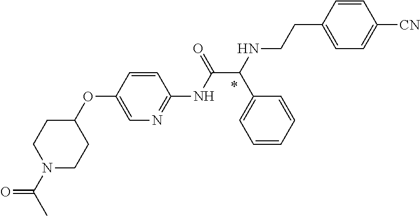 [SMILES: CC(=O)N1CCC(Oc2ccc(NC(=O)C(NCCc3ccc(C#N)cc3)c3ccccc3)nc2)CC1]

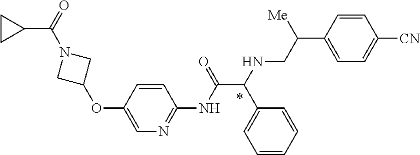 [SMILES: CC(CNC(C(=O)Nc1ccc(OC2CN(C(=O)C3CC3)C2)cn1)c1ccccc1)c1ccc(C#N)cc1]

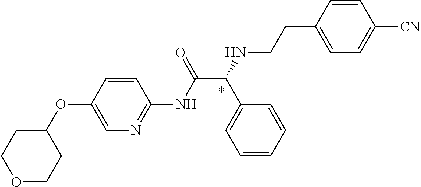 [SMILES: N#Cc1ccc(CCN[C@@H](C(=O)Nc2ccc(OC3CCOCC3)cn2)c2ccccc2)cc1]

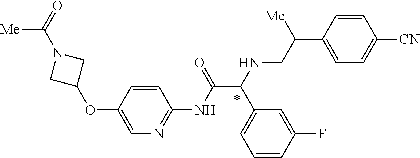 [SMILES: CC(=O)N1CC(Oc2ccc(NC(=O)C(NCC(C)c3ccc(C#N)cc3)c3cccc(F)c3)nc2)C1]

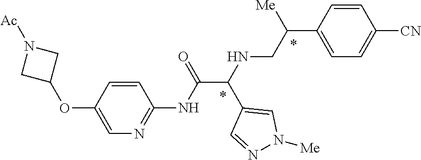 [SMILES: CC(=O)N1CC(Oc2ccc(NC(=O)C(NCC(C)c3ccc(C#N)cc3)c3cnn(C)c3)nc2)C1]

or a pharmaceutically acceptable salt thereof.

### [Item 81]

The pharmaceutical composition of item 42, wherein the low molecular weight compound is a compound represented by formula (21)

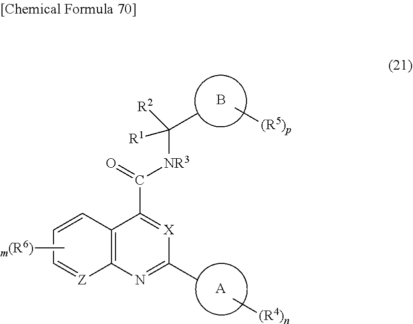

wherein
X is CH or N;
Z is N, CH, or CR⁶;
ring A is monocyclic aryl, bicyclic aryl, monocyclic heterocyclyl, or bicyclic heterocyclyl;
ring B is 5-membered N-containing heteroaryl;
R¹ and R² are each independently hydrogen, C₁₋₆ alkyl, a halogen atom, CN, —C(O)R^{1a}, —C(O)OR^{1a}, —C(O)N(R^{1a})₂, —N(R^{1a})₂, —N(R^{1a})C(O)R^{1a}, —N(R^{1a})C(O)OR^{1a}, —N(R^{1a})C(O)N(R^{1a}), —N(R^{1a})S(O)OR^{1a}, —OR^{1a}, —OC(O)R^{1a}, —OC(O)N(R^{1a})₂, —SR^{1a}, —S(O)R^{1a}, —S(O)₂R^{1a}, —S(O)N(R^{1a}), or —S(O)₂N(R^{1a});
R^{1a} is each independently hydrogen, C₁₋₆ alkyl, C₂₋₆ alkenyl, C₂₋₆ alkynyl, carbocyclyl, or heterocyclyl, or wherein two R^{1a}, together with the nitrogen atom to which they are attached, may form a 4- to 7-membered ring (wherein the 4- to 7-membered ring may contain 1 to 2 each independently selected nitrogen atoms, oxygen atoms, or sulfur atoms);
R³ is hydrogen or C₁₋₆ alkyl;
R⁴ is each independently C₁₋₆ alkyl, C₂₋₆ alkenyl, C₂₋₆ alkynyl, carbocyclyl, heterocyclyl, a halogen atom, CN, —C(O)R^{4a}, —C(O)OR^{4a}, —C(O)N(R^{4a})₂, —N(R^{4a})₂, —N(R^{4a})C(O)R^{4a}, —N(R^{4a})C(O)OR^{4a}, —N(R^{4a})C(O)N(R^{4a})₂, —N(R^{4a})S(O)OR^{4a}, —OR^{4a}, —OC(O)R^{4a}, —OC(O)N(R^{4a})₂, —SR^{4a}, —S(O)R^{4a}, —S(O)₂R^{4a}, —S(O)N(R^{a})₂, —S(O)₂N(R^{4a})₂, or —P(O)(R^{4a})₂;
R^{4a} is each independently hydrogen, C₁₋₆ alkyl, C₂₋₆ alkenyl, C₂₋₆ alkynyl, carbocyclyl, heterocyclyl, or —P(O)(R^{7a})₂, or wherein two R^{4a}, together with the nitrogen atom to which they are attached, may form a 4- to 7-membered ring (wherein the groups may contain 1 to 2 each independently selected nitrogen atoms, oxygen atoms, or sulfur atoms);
R⁵ is each independently C₁₋₆ alkyl or carbocyclyl, or wherein two R⁵, together with the atom to which they are attached, may form a 4- to 7-membered ring (wherein the 4- to 7-membered ring may contain 1 to 2 each independently selected nitrogen atoms, oxygen atoms, or sulfur atoms);
R⁶ is each independently C₁₋₆ alkyl, C₂₋₆ alkenyl, C₂₋₆ alkynyl, carbocyclyl, heterocyclyl, a halogen atom, —CN, —C(O)R^{6a}, —C(O)OR^{6a}, —C(O)N(R^{6a})₂, —N(R^{6a})₂, —N(R^{6a})C(O)R^{6a}, —N(R^{6a})C(O)OR^{6a}, —N(R^{6a})C(O)N(R^{6a})₂, —N(R^{6a})S(O)OR^{6a}, —OR^{6a}, —OC(O)R^{6a}, —OC(O)N(R^{6a})₂, —SR^{6a}, —S(O)R^{6a}, —S(O)₂R^{6a}, —S(O)N(R^{6a})₂, —S(O)₂N(R^{6a})₂, or —P(O)(R^{6a})₂;
R^{6a} is each independently hydrogen, C₁₋₆ alkyl, C₂₋₆ alkenyl, C₂₋₆ alkynyl, carbocyclyl, or heterocyclyl, or wherein two R^{6a}, together with the nitrogen atom to which they are attached, may form a 4- to 7-membered ring (wherein the 4- to 7-membered ring may contain 1 to 2 each independently selected nitrogen atoms, oxygen atoms, or sulfur atoms);
m is 0, 1, 2, or 3;
p is 0, 1, 2, or 3;
n is 0, 1, 2, 3, 4, 5, or 6;
the C₁₋₆ alkyl, C₂₋₆ alkenyl, C₂₋₆ alkynyl, carbocyclyl, and heterocyclyl may be substituted with one or two or more independent R⁷, halogen atoms, —CN, —C(O)R⁷, —C(O)OR⁷, —C(O)N(R⁷)₂, —N(R⁷)₂, —N(R⁷)C(O)R⁷, —N(R⁷)C(O)OR⁷, —N(R⁷)C(O)N(R⁷)₂, —N(R⁷)S(O)OR⁷, —OR⁷, —OC(O)R⁷, —OC(O)N(R⁷)₂, —SR⁷, —S(O)R⁷, —S(O)₂R⁷, —S(O)₂N(R⁷)₂, —S(O)₂N(R⁷)₂, or —P(O)(R⁷)₂;
R⁷ is each independently hydrogen, C₁₋₆ alkyl, C₂₋₆ alkenyl, C₂₋₆ alkynyl, carbocyclyl, or heterocyclyl, wherein the C₁₋₆ alkyl, the C₂₋₆ alkenyl, the C₂₋₆ alkynyl, the carbocyclyl, or the heterocyclyl is optionally substituted with one or two or more substituents selected from R^{7a}, a halogen atom, —CN, —C(O)R^{7a}, —C(O)OR^{7a}, —C(O)N(R^{7a})₂, —N(R^{7a})₂, —N(R^{7a})C(O)R^{7a}, —N(R^{7a})C(O)OR^{7a}, —N(R^{7a})C(O)N(R^{7a})₂, —N(R^{7a})S(O)OR^{7a}, —OR^{7a}, —OC(O)R^{7a}, —OC(O)N(R^{7a})₂, —SR^{7a}, —S(O)R^{7a}, —S(O)₂R^{7a}, —S(O)N(R^{7a})₂, —S(O)₂N(R^{7a})₂, and —P(O)(R^{7a})₂; and
R^{7a} is each independently hydrogen or C₁₋₄ alkyl,
   or a prodrug thereof or a pharmaceutically acceptable salt thereof.

### [Item 82]

The pharmaceutical composition of item 42, wherein the low molecular weight compound is a compound represented by

TABLE 40

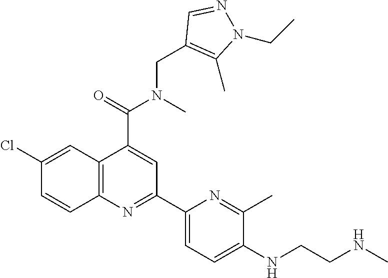 [SMILES: CCn1ncc(CN(C)C(=O)c2cc(-c3ccc(NCCNC)c(C)n3)nc3ccc(Cl)cc23)c1C]

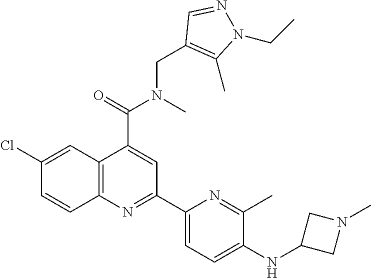 [SMILES: CCn1ncc(CN(C)C(=O)c2cc(-c3ccc(NC4CN(C)C4)c(C)n3)nc3ccc(Cl)cc23)c1C]

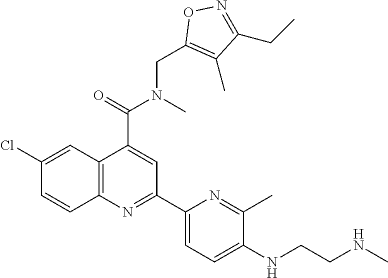 [SMILES: CCc1noc(CN(C)C(=O)c2cc(-c3ccc(NCCNC)c(C)n3)nc3ccc(Cl)cc23)c1C]

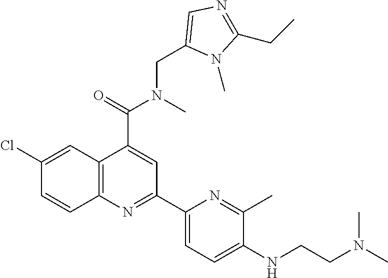 [SMILES: CCc1ncc(CN(C)C(=O)c2cc(-c3ccc(NCCN(C)C)c(C)n3)nc3ccc(Cl)cc23)n1C]

or a pharmaceutically acceptable salt thereof.

### [Item 83]

The pharmaceutical composition of item 42, wherein the low molecular weight compound is a compound represented by formula (22)

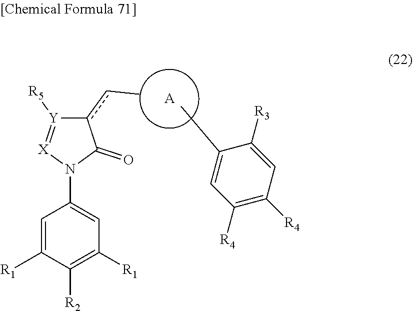

wherein
ring A is 5- or 6-membered aryl, or heteroaryl comprising a nitrogen, oxygen, or sulfur atom, and 1 to 4 carbons;
R¹ is hydrogen or halogen;
R² is a hydroxyl group, carboxyl, C₁₋₄ sulfoalkyl, boronic acid, or nitrogen-containing 5-membered heteroaryl;
R³ is trifluoromethyl, trifluoromethoxy, phosphinyl, nitro, difluoromethyl, or cyclopentanone-containing carbocyclyl;
R⁴ is hydrogen or methyl;
R⁵ is hydrogen, C₁₋₄ alkyl, or cycloalkyl;
X is —C(O)— or —N═;
Y is a carbon atom, a sulfur atom, or —NH—, and
if X is —N═, Y is a carbon atom, and there is a double bond between X and Y, and if X is —C(O)—, Y is a sulfur atom or —NH—, and there is a single bond between X and Y, but an R⁵ group is absent,
   or a prodrug thereof or a pharmaceutically acceptable salt thereof.

### [Item 84]

The pharmaceutical composition of item 42, wherein the low molecular weight compound is a compound represented by

TABLE 41

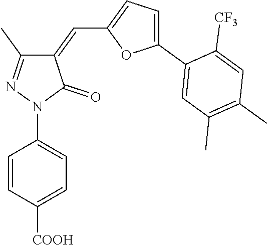

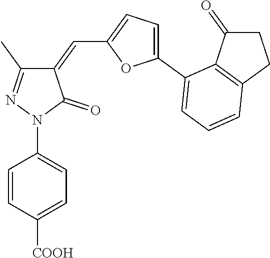 [SMILES: CC1=NN(c2ccc(C(=O)O)cc2)C(=O)/C1=C\c1ccc(-c2cccc3c2C(=O)CC3)o1]

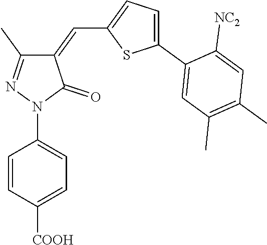

or a pharmaceutically acceptable salt thereof.

### [Item 85]

The pharmaceutical composition of item 42, wherein the low molecular weight compound is a compound represented by formula (23)

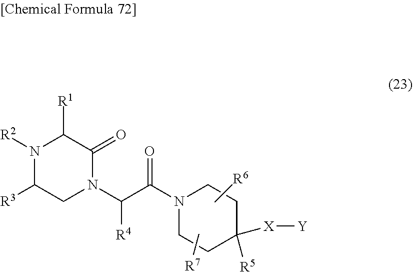

wherein
R¹ is hydrogen, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with cycloalkyl, aryl, or heteroaryl (wherein the cycloalkyl, the aryl, or the heteroaryl is optionally substituted with halogen, C₁₋₄ alkyl, or C₃₋₅ cycloalkyl);
R² is each independently hydrogen, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkylene-O—C₁₋₃ alkylene-O—C₁₋₃ alkylene, C₁₋₅ alkyl-NHCOR¹³, or C₁₋₃ alkyl substituted with cycloalkyl, aryl, or heteroaryl (wherein the cycloalkyl, the aryl, or the heteroaryl is optionally substituted with a halogen atom, C₁₋₄ alkyl, or C₃₋₅ cycloalkyl); with the proviso that if R² is C(O)NR¹⁵R¹⁵, both R¹⁵ may form a ring comprising a nitrogen atom of NR¹⁵R¹⁵ (wherein the ring may further comprise a heteroatom selected from an oxygen atom and a nitrogen atom, and if a nitrogen atom is contained, it is optionally substituted with R⁸);
R³ and R⁷ are each independently hydrogen, C₁₋₇ alkyl, C₂₋₇ alkenyl, C₂₋₇ alkynyl, C₃₋₇ cycloalkyl, or C₄₋₇ cycloalkenyl, which are optionally substituted with a halogen atom, OR⁸, NR⁸R¹¹, or C₁₋₃ alkyl substituted with aryl and heteroaryl (wherein the aryl and the heteroaryl are optionally substituted with a halogen atom, C₁₋₄ alkyl, or C₃₋₅ heteroalkyl);
R⁴ is C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with cycloalkyl, aryl, or heteroaryl (wherein the cycloalkyl, the aryl, or the heteroaryl is optionally substituted with a halogen atom, C₁₋₄ alkyl, or C₃₋₅ heteroalkyl);
R⁵ is hydrogen, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸, wherein R⁵, together with X and Y, may form a ring that may comprise a carbonyl group;
R⁶ is hydrogen, C₁₋₇ alkyl, C₂₋₇ alkenyl, C₂₋₇ alkynyl, C₃₋₇ cycloalkyl, C₄₋₇ cycloalkenyl (wherein the C₁₋₇ alkyl, the C₂₋₇ alkenyl, the C₂₋₇ alkynyl, the C₃₋₇ cycloalkyl, or the C₄₋₇ cycloalkenyl is optionally substituted with a halogen atom, OR⁸, NR⁸R¹¹, C₁₋₃ alkyl substituted with C(O)NR⁸R¹¹, or C₁₋₃ alkyl substituted with aryl or heteroaryl (wherein the aryl or the heteroaryl is optionally substituted with a halogen atom, C₁₋₄ alkyl, or C₃₋₅ cycloalkyl), wherein R⁶ may form a ring with any part of X, or is imidazolidinone;
R⁸ and R¹¹ are each independently hydrogen, C₁₋₇ alkyl, C₂₋₇ alkenyl, C₂₋₇ alkynyl, C₃₋₇ cycloalkyl, or C₄₋₇ cycloalkenyl;
X is a bond, C₁₋₇ alkylene, C₂₋₇ alkenylene, C₂₋₇ alkynylene, C₃₋₉ cycloalkylene, C₄₋₆ cycloalkenylene, —O—, C₁₋₃ alkylene-O—, —O—C₁₋₇ alkylene, —O—C₃₋₉ cycloalkylene, C₁₋₃ alkylene-O—C₁₋₇ alkylene, C₁₋₇ heteroalkylene, or —S—C₁₋₇ alkylene, wherein X, together with R¹, R⁶, and Y, may form a polycyclic system or a ring that may comprise a carbonyl group;
Y is hydrogen, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1, or 2, SO₂NR¹⁰R¹², NR¹⁰SO₂R¹⁰, NR¹⁰R¹², HNCOR⁸, CN, C₃₋₇ cycloalkyl that may comprise a nitrogen atom optionally substituted with R⁸ or an oxygen atom within a ring, S-aryl, O-aryl, S-heteroaryl, O-heteroaryl (wherein the S-aryl, the O-aryl, the S-heteroaryl, or the O-heteroaryl is optionally substituted with one or two or more R⁹ or R¹⁴), aryl, or heteroaryl (wherein the aryl or the heteroaryl is optionally substituted with one or two or more R⁸); wherein Y may form a ring that may comprise a carbonyl group at any position on X or R⁵, but if Y is C(O)NR¹⁰R¹² or NR¹⁰R¹², R¹⁰ and R¹² may form a ring comprising a nitrogen atom of NR¹⁰R¹² (wherein the ring may further comprise a heteroatom selected from an oxygen atom and a nitrogen atom, and if a nitrogen atom is contained, it is optionally substituted with R⁸);
R⁹ is hydrogen, a halogen atom, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR₈C(O)NR⁸R¹¹, OC(O)NR⁸R¹¹, SO₂NR⁸R¹¹, NR⁸SO₂R⁸, OR⁸, NR⁸R¹¹, or S(O)nR⁸ wherein n is 0, 1, or 2;
R¹⁰ and R¹² are each independently hydrogen, C₁₋₇ alkyl, C₂₋₇ alkenyl, C₂₋₇ alkynyl, C₃₋₇ cycloalkyl, or C₄₋₇ cycloalkenyl, C₁₋₃ alkylene-O—C₁₋₃ alkylene-O—C₁₋₃ alkylene, C₁₋₃ alkyl-aryl, or C₁₋₃ alkyl-heteroaryl, wherein R¹⁰ and R¹² are optionally substituted with a halogen atom, OR₈, or NR₈R¹¹;
R¹³ is C₁₋₇ alkyl substituted with a bicycle that may comprise at least one heteroatom or carbonyl group;
R¹⁴ is hydrogen, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with aryl or heteroaryl (wherein the aryl or the heteroaryl is optionally substituted with a halogen atom, C₁₋₄ alkyl, or C₃₋₅ heteroalkyl); and
R¹⁵ is each independently hydrogen, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸,
   or a prodrug thereof or a pharmaceutically acceptable salt thereof.

### [Item 86]

The pharmaceutical composition of item 42, wherein the low molecular weight compound is a compound represented by

TABLE 42

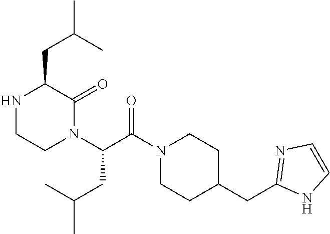 [SMILES: CC(C)C[C@@H]1NCCN([C@@H](CC(C)C)C(=O)N2CCC(Cc3ncc[nH]3)CC2)C1=O]

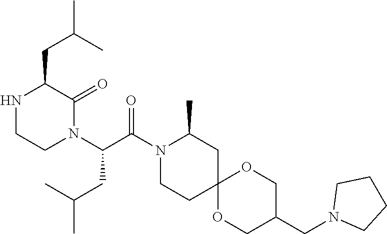 [SMILES: CC(C)C[C@@H]1NCCN([C@@H](CC(C)C)C(=O)N2CCC3(C[C@@H]2C)OCC(CN2CCCC2)CO3)C1=O]

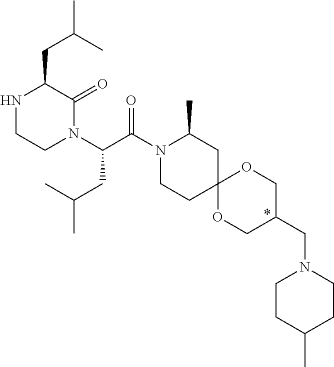 [SMILES: CC(C)C[C@@H]1NCCN([C@@H](CC(C)C)C(=O)N2CCC3(C[C@@H]2C)OCC(CN2CCC(C)CC2)CO3)C1=O]

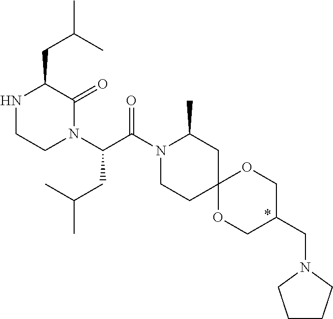 [SMILES: CC(C)C[C@@H]1NCCN([C@@H](CC(C)C)C(=O)N2CCC3(C[C@@H]2C)OCC(CN2CCCC2)CO3)C1=O]

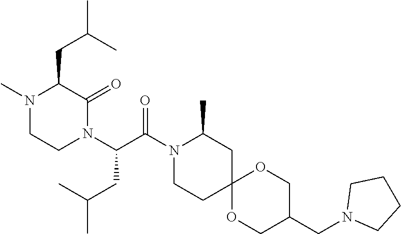 [SMILES: CC(C)C[C@H]1C(=O)N([C@@H](CC(C)C)C(=O)N2CCC3(C[C@@H]2C)OCC(CN2CCCC2)CO3)CCN1C]

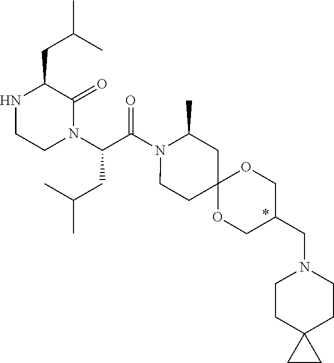 [SMILES: CC(C)C[C@@H]1NCCN([C@@H](CC(C)C)C(=O)N2CCC3(C[C@@H]2C)OCC(CN2CCC4(CC2)CC4)CO3)C1=O]

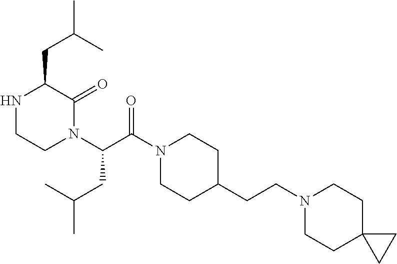 [SMILES: CC(C)C[C@@H]1NCCN([C@@H](CC(C)C)C(=O)N2CCC(CCN3CCC4(CC3)CC4)CC2)C1=O]

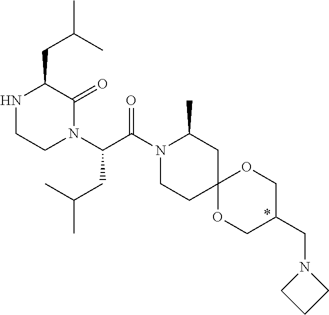 [SMILES: CC(C)C[C@@H]1NCCN([C@@H](CC(C)C)C(=O)N2CCC3(C[C@@H]2C)OCC(CN2CCC2)CO3)C1=O]

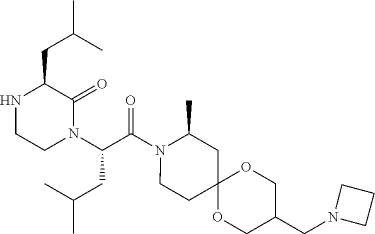 [SMILES: CC(C)C[C@@H]1NCCN([C@@H](CC(C)C)C(=O)N2CCC3(C[C@@H]2C)OCC(CN2CCC2)CO3)C1=O]

or a pharmaceutically acceptable salt thereof.

### [Item 87]

A pharmaceutical composition for use in treating and/or preventing cancer, comprising a CBP/P300 inhibitor as an active ingredient, characterized by being administered to a subject comprising at least one selected from the group consisting of a dysfunction of an SWI/SNF complex, and lack of or attenuation of expression of an SWI/SNF complex protein.

### [Item 88]

The pharmaceutical composition of item 87, wherein the subject comprising at least one selected from the group consisting of a dysfunction of an SWI/SNF complex, and lack of or attenuation of expression of an SWI/SNF complex protein is determined by steps comprising
(1) a step comprising at least one selected from the group consisting of a step of detecting a mutation in an SWI/SNF complex gene of a cancer cell obtained from the subject, and a step of measuring expression of an SWI/SNF complex protein, and
(2) a step of determining that the subject comprises at least one selected from the group consisting of a dysfunction of an SWI/SNF complex, and lack of or attenuation of expression of an SWI/SNF complex protein based on at least one selected from the group consisting of the presence/absence of a mutation in an SWI/SNF complex gene and a result of expression of an SWI/SNF complex protein detected in (1).

### [Item 89]

The pharmaceutical composition of item 88, wherein the SWI/SNF complex is a BAF complex, the SWI/SNF complex gene is a BAF complex gene, and the SWI/SNF complex protein is a BAF complex protein.

### [Item 90]

The pharmaceutical composition of item 89, wherein
the BAF complex gene comprises at least one gene selected from the group consisting of an SMARC gene, an SS18-SSX fusion gene, and an ARID gene, and
the BAF complex protein comprises at least one protein selected from the group consisting of an SMARC protein, an SS18-SSX fusion protein, and an ARID protein.

### [Item 91]

The pharmaceutical composition of item 89 or 90, wherein
the BAF complex gene is an SMARC gene, and
the BAF complex protein is an SMARC protein.

### [Item 92]

The pharmaceutical composition of item 90 or 91, wherein
the SMARC gene comprises at least one gene selected from the group consisting of an SMARCB1 gene, an SMARCA2 gene, and an SMARCA4 gene, and
the SMARC protein comprises at least one protein selected from the group consisting of an SMARCB1 protein, an SMARCA2 protein, and an SMARCA4 protein.

### [Item 93]

The pharmaceutical composition of item 90 or 91, wherein
the SMARC gene is an SMARCB1 gene, and
the SMARC protein is an SMARCB1 protein.

### [Item 94]

The pharmaceutical composition of item 90 or 91, wherein the SMARC gene is an SMARCA2 gene, and the SMARC protein is an SMARCA2 protein.

### [Item 95]

The pharmaceutical composition of item 90 or 91, wherein the SMARC gene is an SMARCA4 gene, and the SMARC protein is an SMARCA4 protein.

### [Item 96]

The pharmaceutical composition of item 90 or 91, wherein the SMARC gene comprises an SMARCA2 gene and an SMARCA4 gene, and the SMARC protein comprises an SMARCA2 protein and an SMARCA4 protein.

### [Item 97]

The pharmaceutical composition of any one of items 87 to 96, wherein the cancer is SMARC deficient cancer.

### [Item 96]

The pharmaceutical composition of item 97, wherein the SMARC deficient cancer is SMARCB1 deficient cancer.

### [Item 99]

The pharmaceutical composition of item 98, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

### [Item 100]

The pharmaceutical composition of item 98, wherein the SMARCB1 deficient cancer is malignant rhabdoid tumor.

### [Item 101]

The pharmaceutical composition of item 97, wherein the SMARC deficient cancer is SMARCA2 deficient cancer.

### [Item 102]

The pharmaceutical composition of item 101, wherein the SMARCA2 deficient cancer comprises at least one selected from the group consisting of pulmonary adenocarcinoma, large cell lung carcinoma, lung neuroendocrine tumor, esophageal cancer, gastroesophageal junction cancer, and malignant rhabdoid tumor.

### [Item 103]

The pharmaceutical composition of item 101, wherein the SMARCA2 deficient cancer is pulmonary adenocarcinoma.

### [Item 104]

The pharmaceutical composition of item 97, wherein the SMARC deficient cancer is SMARCA4 deficient cancer.

### [Item 105]

The pharmaceutical composition of item 104, wherein the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

### [Item 106]

The pharmaceutical composition of item 104, wherein the SMARCA4 deficient cancer is pulmonary adenocarcinoma.

### [Item 107]

The pharmaceutical composition of item 97, wherein the SMARC deficient cancer is SMARCA2/A4 deficient cancer.

### [Item 108]

The pharmaceutical composition of item 107, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

### [Item 109]

The pharmaceutical composition of item 107, wherein the SMARCA2/A4 deficient cancer is pulmonary adenocarcinoma.

### [Item 110]

The pharmaceutical composition of item 89, wherein
the BAF complex gene is an ARID gene, and
the BAF complex protein is an ARID protein.

### [Item 111]

The pharmaceutical composition of item 110, wherein
the ARID gene comprises at least one gene selected from the group consisting of an ARID1A gene and an ARID1B gene, and
the ARID protein comprises at least one protein selected from the group consisting of an ARID1A protein and an ARID1B protein.

### [Item 112]

The pharmaceutical composition of item 110, wherein
the ARID gene is an ARID1A gene, and
the ARID protein is an ARID1A protein.

### [Item 113]

The pharmaceutical composition of item 110, wherein
the ARID gene is an ARID1B gene, and
the ARID protein is an ARID1B protein.

### [Item 114]

The pharmaceutical composition of item 110, wherein the ARID gene is an ARID1A gene and an ARID1B gene, and the ARID protein is an ARID1A protein and an ARID1B protein.

### [Item 115]

The pharmaceutical composition of any one of items 87 to 90 and 110 to 114, wherein the cancer is ARID deficient cancer.

### [Item 116]

The pharmaceutical composition of item 115, wherein the ARID deficient cancer is ARID1A deficient cancer.

### [Item 117]

The pharmaceutical composition of item 115, wherein the ARID1A deficient cancer comprises at least one selected from the group consisting of ovarian cancer, gastric cancer, bile duct cancer, pancreatic cancer, uterine cancer, neuroblastoma, colon cancer, and bladder cancer.

### [Item 118]

The pharmaceutical composition of item 115, wherein the ARID1A deficient cancer is ovarian cancer.

### [Item 119]

The pharmaceutical composition of item 115, wherein the ARID deficient cancer is ARID1B deficient cancer.

### [Item 120]

The pharmaceutical composition of item 119, wherein the ARID1B deficient cancer comprises at least one selected from the group consisting of ovarian cancer, colon cancer, pancreatic cancer, liver cancer, melanoma, breast cancer, medulloblastoma, uterine cancer, bladder cancer, and gastric cancer.

### [Item 121]

The pharmaceutical composition of item 119, wherein the ARID1B deficient cancer is ovarian cancer.

### [Item 122]

The pharmaceutical composition of item 115, wherein the ARID deficient cancer is ARID1A/1B deficient cancer.

### [Item 123]

The pharmaceutical composition of item 122, wherein the ARID1A/1B deficient cancer comprises at least one selected from the group consisting of ovarian cancer, colon cancer, uterine cancer, neuroblastoma, bladder cancer, and gastric cancer.

### [Item 124]

The pharmaceutical composition of item 122, wherein the ARID1A/1B deficient cancer is ovarian cancer.

### [Item 125]

The pharmaceutical composition of item 89 or 90,
wherein the BAF complex gene is an SS18-SSX fusion gene, and the BAF complex protein is an SS18-SSX fusion protein.

### [Item 126]

The pharmaceutical composition of any one of items 87 to 90 and 125, wherein the cancer is SS18-SSX fusion cancer.

### [Item 127]

The pharmaceutical composition of item 126, wherein the SS18-SSX fusion cancer is synovial sarcoma or Ewing's sarcoma.

### [Item 128]

The pharmaceutical composition of item 126, wherein the SS18-SSX fusion cancer is synovial sarcoma.

### [Item 129]

The pharmaceutical composition of any one of items 87 to 128, wherein the CBP/P300 inhibitor reduces expression of CBP and/or P300, and/or suppresses a function of CBP and/or P300.

### [Item 130]

The pharmaceutical composition of any one of items 87 to 129, wherein the CBP/P300 inhibitor is a nucleic acid or a low molecular weight compound.

### [Item 131]

The pharmaceutical composition of any one of items 87 to 130, wherein the CBP/P300 inhibitor is a low molecular weight compound.

### [Item 132]

A pharmaceutical composition comprising a CBP/P300 inhibitor in combination with at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, other antitumor agents, and agents classified as other antitumor agents.

### [Item 133]

A pharmaceutical composition comprising a CBP/P300 inhibitor for use in treating and/or preventing cancer by concomitantly using at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, and agents classified as other antitumor agents.

[Item 134]A method for assisting prediction of efficacy of a CBP/P300 inhibitor on a subject, comprising at least one selected from the group consisting of detecting a dysfunction of an SWI/SNF complex in a cancer cell of the subject, and measuring expression of an SWI/SNF complex protein.

### [Item 135]

The method of item 134, wherein the at least one selected from the group consisting of detecting a dysfunction of an SWI/SNF complex in a cancer cell, and measuring expression of an SWI/SNF complex protein is determined by steps comprising
(1) at least one selected from the group consisting of a step of detecting a mutation in an SWI/SNF complex gene of a cancer cell obtained from the subject and a step of measuring expression of an SWI/SNF complex protein, and
(2) a step of determining that the subject comprises at least one selected from the group consisting of a dysfunction of an SWI/SNF complex, and lack of or attenuation of expression of an SWI/SNF complex protein based on at least one selected from the group consisting of the presence/absence of a mutation in an SWI/SNF complex gene and a result of expression of an SWI/SNF complex protein detected in (1).

### [Item 136]

A method of using at least one selected from the group consisting of the presence/absence or level of a mutation in an SWI/SNF complex gene in a cancer cell of a subject and the presence/absence or level of expression of an SWI/SNF complex protein as an indicator for predicting efficacy of a CBP/P300 inhibitor on the subject.

### [Item 137]

The method of item 135 or 136, wherein the SWI/SNF complex is a BAF complex, the SWI/SNF complex gene is a BAF complex gene, and the SWI/SNF complex protein is a BAF complex protein.

### [Item 138]

The method of item 137, wherein
the BAF complex gene comprises at least one gene selected from the group consisting of an SMARC gene, an SS18-SSX fusion gene, and an ARID gene, and
the BAF complex protein comprises at least one protein selected from the group consisting of an SMARC protein, an SS18-SSX fusion protein, and an ARID protein.

### [Item 139]

The method of item 137 or 138, wherein
the BAF complex gene is an SMARC gene, and
the BAF complex protein is an SMARC protein.

### [Item 140]

The method of item 138 or 139, wherein the SMARC gene comprises at least one gene selected from the group consisting of an SMARCB1 gene, an SMARCA2 gene, and an SMARCA4 gene, and the SMARC protein comprises at least one protein selected from the group consisting of an SMARCB1 protein, an SMARCA2 protein, and an SMARCA4 protein.

### [Item 141]

The method of item 138 or 139, wherein the SMARC gene is an SMARCB1 gene, and the SMARC protein is an SMARCB1 protein.

### [Item 142]

The method of item 138 or 139, wherein the SMARC gene is an SMARCA2 gene, and the SMARC protein is an SMARCA2 protein.

### [Item 143]

The method of item 138 or 139, wherein the SMARC gene is an SMARCA4 gene, and the SMARC protein is an SMARCA4 protein.

### [Item 144]

The method of item 138 or 139, wherein the SMARC gene comprises an SMARCA2 gene and an SMARCA4 gene, and the SMARC protein comprises an SMARCA2 protein and an SMARCA4 protein.

### [Item 145]

The method of any one of items 134 to 144, wherein the cancer is SMARC deficient cancer.

### [Item 146]

The method of item 145, wherein the SMARC deficient cancer is SMARCB1 deficient cancer.

### [Item 147]

The method of item 146, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

### [Item 148]

The method of item 146, wherein the SMARCB1 deficient cancer is malignant rhabdoid tumor.

### [Item 149]

The method of item 145, wherein the SMARC deficient cancer is SMARCA2 deficient cancer.

### [Item 150]

The method of item 149, wherein the SMARCA2 deficient cancer is pulmonary adenocarcinoma, large cell lung carcinoma, lung neuroendocrine tumor, esophageal cancer, gastroesophageal junction cancer, or malignant rhabdoid tumor.

### [Item 151]

The method of item 149, wherein the SMARCA2 deficient cancer is pulmonary adenocarcinoma.

### [Item 152]

The method of item 145, wherein the SMARC deficient cancer is SMARCA4 deficient cancer.

### [Item 153]

The method of item 152, wherein the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

### [Item 154]

The method of item 152, wherein the SMARCA4 deficient cancer is pulmonary adenocarcinoma.

### [Item 155]

The method of item 145, wherein the SMARC deficient cancer is SMARCA2/A4 deficient cancer.

### [Item 156]

The method of item 155, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

### [Item 157]

The method of item 155, wherein the SMARCA2/A4 deficient cancer is pulmonary adenocarcinoma.

### [Item 158]

The method of item 137 or 138, wherein
the BAF complex gene is an ARID gene, and
the BAF complex protein is an ARID protein.

### [Item 159]

The method of item 158, wherein the ARID gene comprises at least one gene selected from the group consisting of an ARID1A gene and an ARID1B gene, and the ARID protein comprises at least one protein selected from the group consisting of an ARID1A protein and an ARID1B protein.

### [Item 160]

The method of item 158, wherein the ARID gene is an ARID1A gene, and the ARID protein is an ARID1A protein.

### [Item 161]

The method of item 158, wherein the ARID gene is an ARID1B gene, and the ARID protein is an ARID1B protein.

### [Item 162]

The method of item 158, wherein the ARID gene comprises an ARID1A gene and an ARID1B gene, and the ARID protein comprises an ARID1A protein and an ARID1B protein.

### [Item 163]

The method of any one of items 134 to 138 and 158 to 162, wherein the cancer is ARID deficient cancer.

### [Item 164]

The method of item 163, wherein the ARID deficient cancer is ARID1A deficient cancer.

### [Item 165]

The method of item 164, wherein the ARID1A deficient cancer comprises at least one selected from the group consisting of ovarian cancer, gastric cancer, bile duct cancer, pancreatic cancer, uterine cancer, neuroblastoma, colon cancer, and bladder cancer.

### [Item 166]

The method of item 164, wherein the ARID1A deficient cancer is ovarian cancer.

### [Item 167]

The method of item 163, wherein the ARID deficient cancer is ARID1B deficient cancer.

### [Item 168]

The method of item 167, wherein the ARID1B deficient cancer comprises at least one selected from the group consisting of ovarian cancer, colon cancer, pancreatic cancer, liver cancer, melanoma, breast cancer, medulloblastoma, uterine cancer, bladder cancer, and gastric cancer.

### [Item 169]

The method of item 167, wherein the ARID1B deficient cancer is ovarian cancer.

### [Item 170]

The method of item 163, wherein the ARID deficient cancer is ARID1A/1B deficient cancer.

### [Item 171]

The method of item 170, wherein the ARID1A/1B deficient cancer comprises at least one selected from the group consisting of ovarian cancer, colon cancer, uterine cancer, neuroblastoma, bladder cancer, and gastric cancer.

### [Item 172]

The method of item 170, wherein the ARID1A/1B deficient cancer is ovarian cancer.

### [Item 173]

The method of item 137 or 138, wherein the BAF complex gene is an SS18-SSX fusion gene, and the BAF complex protein is an SS18-SSX fusion gene protein.

### [Item 174]

The method of any one of items 134 to 138 and 173,
wherein the cancer is SS18-SSX fusion cancer.

### [Item 175]

The method of item 174, wherein the SS18-SSX fusion cancer is synovial sarcoma or Ewing's sarcoma.

### [Item 176]

The method of item 174, wherein the SS18-SSX fusion cancer is synovial sarcoma.

### [Item 177]

The method of any one of items 134 to 176, wherein the CBP/P300 inhibitor reduces expression of CBP and/or P300, and/or suppresses a function of CBP and/or P300.

### [Item 178]

The method of any one of items 134 to 177, wherein the CBP/P300 inhibitor is a nucleic acid or a low molecular weight compound.

### [Item 179]

The method of any one of items 134 to 178, wherein the CBP/P300 inhibitor is a low molecular weight compound.

### [Item 180]

A pharmaceutical composition for use in treating and/or preventing cancer, comprising an SWI/SNF complex inhibitor.

### [Item 181]

The pharmaceutical composition of item 180, wherein the cancer is CBP/P300 deficient cancer.

### [Item 182]

The pharmaceutical composition of item 181, wherein the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

### [Item 183]

The pharmaceutical composition of any one of items 180 to 182, wherein the SWI/SNF complex inhibitor is a BAF complex inhibitor.

### [Item 184]

The pharmaceutical composition of item 183, wherein the BAF complex inhibitor is at least one inhibitor selected from the group consisting of an SMARC inhibitor and an ARID inhibitor.

### [Item 185]

The pharmaceutical composition of item 183, wherein the BAF complex inhibitor is an SMARC inhibitor.

### [Item 186]

The pharmaceutical composition of item 184 or 185, wherein the SMARC inhibitor comprises at least one inhibitor selected from the group consisting of an SMARCB1 inhibitor, an SMARCA2 inhibitor, an SMARCA4 inhibitor, and an SMARCA2/A4 inhibitor.

### [Item 187]

The pharmaceutical composition of item 184 or 185, wherein the SMARC inhibitor is an SMARCB1 inhibitor.

### [Item 188]

The pharmaceutical composition of item 187, wherein the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

### [Item 189]

The pharmaceutical composition of item 187, wherein the SMARCB1 inhibitor is a low molecular weight compound that inhibits a function of SMARCB1.

### [Item 190]

The pharmaceutical composition of item 184 or 185, wherein the SMARC inhibitor is an SMARCA2 inhibitor.

### [Item 191]

The pharmaceutical composition of item 190, wherein the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

### [Item 192]

The pharmaceutical composition of item 190, wherein the SMARCA2 inhibitor is a low molecular weight compound that inhibits a function of SMARCA2.

### [Item 193]

The pharmaceutical composition of item 184 or 185, wherein the SMARC inhibitor is an SMARCA4 inhibitor.

### [Item 194]

The pharmaceutical composition of item 193, wherein the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

### [Item 195]

The pharmaceutical composition of item 193, wherein the SMARCA4 inhibitor is a low molecular weight compound that inhibits a function of SMARCA4.

### [Item 196]

The pharmaceutical composition of item 184 or 185, wherein the SMARC inhibitor is an SMARCA2/A4 inhibitor.

### [Item 197]

The pharmaceutical composition of item 196, wherein the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

### [Item 198]

The pharmaceutical composition of item 196, wherein the SMARCA2/4 inhibitor is a low molecular weight compound that inhibits a function of SMARCA2 and SMARCA4.

### [Item 199]

The pharmaceutical composition of item 183 or 184,
wherein the BAF complex inhibitor is an ARID inhibitor.

### [Item 200]

The pharmaceutical composition of item 199, wherein the ARID inhibitor comprises at least one inhibitor selected from the group consisting of an ARID1A inhibitor, an ARID1B inhibitor, and an ARID1A/1B inhibitor.

### [Item 201]

The pharmaceutical composition of item 199, wherein the ARID inhibitor is an ARID1A inhibitor.

### [Item 202]

The pharmaceutical composition of item 201, wherein the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

### [Item 203]

The pharmaceutical composition of item 201, wherein the ARID1A inhibitor is a low molecular weight compound that inhibits a function of ARID1A.

### [Item 204]

The pharmaceutical composition of item 199, wherein the ARID inhibitor is an ARID1B inhibitor.

### [Item 205]

The pharmaceutical composition of item 204, wherein the ARID1B inhibitor is a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, or precursors thereof.

### [Item 206]

The pharmaceutical composition of item 204, wherein the ARID1B inhibitor is a low molecular weight compound that inhibits a function of ARID1B.

### [Item 207]

The pharmaceutical composition of item 199, wherein the ARID inhibitor is an ARID1A/1B inhibitor.

### [Item 208]

The pharmaceutical composition of item 207, wherein the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

### [Item 209]

The pharmaceutical composition of item 207, wherein the ARID1A/1B inhibitor is a low molecular weight compound that inhibits a function of ARID1A and ARID1B.

### [Item 210]

A pharmaceutical composition for use in treating and/or preventing cancer, comprising an SWI/SNF complex inhibitor as an active ingredient, characterized by being administered to a subject comprising at least one selected from the group consisting of a deficiency of a CBP/P300 gene, and lack of or attenuation of expression of a CBP/P300 protein.

### [Item 211]

The pharmaceutical composition of item 210, wherein the subject comprising at least one selected from the group consisting of a deficiency of a CBP/P300 gene, and lack of or attenuation of expression of a CBP/P300 protein is determined by steps comprising
(1) at least one selected from the group consisting of a step of detecting a mutation in a CBP/P300 gene of a cancer cell obtained from the subject, and a step of measuring expression of a CBP/P300 protein, and
(2) a step of determining that the subject comprises at least one selected from the group consisting of a deficiency of a CBP/P300 gene, and lack of or attenuation of expression of a CBP/P300 gene based on at least one selected from the group consisting of the presence/absence of a mutation in a CBP/P300 gene and a result of expression of a CBP/P300 protein detected in (1).

### [Item 212]

The pharmaceutical composition of item 210 or 211,
wherein the cancer is CBP/P300 deficient cancer.

### [Item 213]

The pharmaceutical composition of item 212, wherein the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

### [Item 214]

The pharmaceutical composition of any one of items 210 to 213, wherein the SWI/SNF complex inhibitor is a BAF complex inhibitor.

### [Item 215]

The pharmaceutical composition of item 214, wherein the BAF complex inhibitor is at least one inhibitor selected from the group consisting of an SMARC inhibitor and an ARID inhibitor.

### [Item 216]

The pharmaceutical composition of item 214, wherein the BAF complex inhibitor is an SMARC inhibitor.

### [Item 217]

The pharmaceutical composition of item 216, wherein the SMARC inhibitor is at least one inhibitor selected from the group consisting of an SMARCB1 inhibitor, an SMARCA2 inhibitor, an SMARCA4 inhibitor, and an SMARCA2/A4 inhibitor.

### [Item 218]

The pharmaceutical composition of item 216, wherein the SMARC inhibitor is an SMARCB1 inhibitor.

### [Item 219]

The pharmaceutical composition of item 218, wherein the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

### [Item 220]

The pharmaceutical composition of item 218, wherein the SMARCB1 inhibitor is a low molecular weight compound that inhibits a function of SMARCB1.

### [Item 221]

The pharmaceutical composition of item 216, wherein the SMARC inhibitor is an SMARCA2 inhibitor.

### [Item 222]

The pharmaceutical composition of item 221, wherein the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

### [Item 223]

The pharmaceutical composition of item 221, wherein the SMARCA2 inhibitor is a low molecular weight compound that inhibits a function of SMARCA2.

### [Item 224]

The pharmaceutical composition of item 216, wherein the SMARC inhibitor is an SMARCA4 inhibitor.

### [Item 225]

The pharmaceutical composition of item 224, wherein the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

### [Item 226]

The pharmaceutical composition of item 224, wherein the SMARCA4 inhibitor is a low molecular weight compound that inhibits a function of SMARCA4.

### [Item 227]

The pharmaceutical composition of item 216, wherein the SMARC inhibitor is an SMARCA2/A4 inhibitor.

### [Item 228]

The pharmaceutical composition of item 227, wherein the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

### [Item 229]

The pharmaceutical composition of item 227, wherein the SMARCA2/4 inhibitor is a low molecular weight compound that inhibits a function of SMARCA2 and SMARCA4.

### [Item 230]

The pharmaceutical composition of item 214 or 215,
wherein the BAF complex inhibitor is an ARID inhibitor.

### [Item 231]

The pharmaceutical composition of item 230, wherein the ARID inhibitor is at least one inhibitor selected from the group consisting of an ARID1A inhibitor, an ARID1B inhibitor, and an ARID1A/1B inhibitor.

### [Item 232]

The pharmaceutical composition of item 230, wherein the ARID inhibitor is an ARID1A inhibitor.

### [Item 233]

The pharmaceutical composition of item 232, wherein the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

### [Item 234]

The pharmaceutical composition of item 232, wherein the ARID1A inhibitor is a low molecular weight compound that inhibits a function of ARID1A.

### [Item 235]

The pharmaceutical composition of item 230, wherein the ARID inhibitor is an ARID1B inhibitor.

### [Item 236]

The pharmaceutical composition of item 235, wherein the ARID1B inhibitor comprises at least one selected from the group consisting of a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, and precursors thereof.

### [Item 237]

The pharmaceutical composition of item 235, wherein the ARID1B inhibitor is a low molecular weight compound that inhibits a function of ARID1B.

### [Item 238]

The pharmaceutical composition of item 230, wherein the ARID inhibitor is an ARID1A/1B inhibitor.

### [Item 239]

The pharmaceutical composition of item 238, wherein the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

### [Item 240]

The pharmaceutical composition of item 238, wherein the ARID1A/1B inhibitor is a low molecular weight compound that inhibits a function of ARID1A and ARID1B.

### [Item 241]

A pharmaceutical composition comprising an SWI/SNF complex inhibitor in combination with at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, other antitumor agents, and agents classified as other antitumor agents.

### [Item 242]

A pharmaceutical composition comprising an SWI/SNF complex inhibitor for use in treating and/or preventing cancer by concomitantly using at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, and agents classified as other antitumor agents.

### [Item 243]

The pharmaceutical composition of item 241 or 242,
wherein the SWI/SNF complex inhibitor is a BAF complex inhibitor.

### [Item 244]

The pharmaceutical composition of item 243, wherein the BAF complex inhibitor comprises at least one inhibitor selected from the group consisting of an SMARC inhibitor and an ARID inhibitor.

### [Item 245]

The pharmaceutical composition of item 243, wherein the BAF complex inhibitor is an SMARC inhibitor.

### [Item 246]

The pharmaceutical composition of item 244 or 245, wherein the SMARC inhibitor comprises at least one inhibitor selected from the group consisting of an SMARCB1 inhibitor, an SMARCA2 inhibitor, an SMARCA4 inhibitor, and an SMARCA2/A4 inhibitor.

### [Item 247]

The pharmaceutical composition of item 244 or 245, wherein the SMARC inhibitor is an SMARCB1 inhibitor.

### [Item 248]

The pharmaceutical composition of item 247, wherein the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

### [Item 249]

The pharmaceutical composition of item 247, wherein the SMARCB1 inhibitor is a low molecular weight compound that inhibits a function of SMARCB1.

### [Item 250]

The pharmaceutical composition of item 244 or 245, wherein the SMARC inhibitor is an SMARCA2 inhibitor.

### [Item 251]

The pharmaceutical composition of item 250, wherein the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

### [Item 252]

The pharmaceutical composition of item 250, wherein the SMARCA2 inhibitor is a low molecular weight compound that inhibits a function of SMARCA2.

### [Item 253]

The pharmaceutical composition of item 245, wherein the SMARC inhibitor is an SMARCA4 inhibitor.

### [Item 254]

The pharmaceutical composition of item 253, wherein the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

### [Item 255]

The pharmaceutical composition of item 253, wherein the SMARCA4 inhibitor is a low molecular weight compound that inhibits a function of SMARCA4.

### [Item 256]

The pharmaceutical composition of item 244 or 245, wherein the SMARC inhibitor is an SMARCA2/A4 inhibitor.

### [Item 257]

The pharmaceutical composition of item 256, wherein the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

### [Item 258]

The pharmaceutical composition of item 256, wherein the SMARCA2/4 inhibitor is a low molecular weight compound that inhibits a function of SMARCA2 and SMARCA4.

### [Item 259]

The pharmaceutical composition of item 243, wherein the BAF complex inhibitor is an ARID inhibitor.

### [Item 260]

The pharmaceutical composition of item 259, wherein the ARID inhibitor comprises at least one inhibitor selected from the group consisting of an ARID1A inhibitor, an ARID1B inhibitor, and an ARID1A/1B inhibitor.

### [Item 261]

The pharmaceutical composition of item 259, wherein the ARID inhibitor is an ARID1A inhibitor.

### [Item 262]

The pharmaceutical composition of item 261, wherein the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

### [Item 263]

The pharmaceutical composition of item 261, wherein the ARID1A inhibitor is a low molecular weight compound that inhibits a function of ARID1A.

### [Item 264]

The pharmaceutical composition of item 259, wherein the ARID inhibitor is an ARID1B inhibitor.

### [Item 265]

The pharmaceutical composition of item 264, wherein the ARID1B inhibitor comprises at least one selected from the group consisting of a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, and precursors thereof.

### [Item 266]

The pharmaceutical composition of item 264, wherein the ARID1B inhibitor is a low molecular weight compound that inhibits a function of ARID1B.

### [Item 267]

The pharmaceutical composition of item 259, wherein the ARID inhibitor is an ARID1A/1B inhibitor.

### [Item 268]

The pharmaceutical composition of item 267, wherein the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

### [Item 269]

The pharmaceutical composition of item 267, wherein the ARID1A/1B inhibitor is a low molecular weight compound that inhibits a function of ARID1A and ARID1B.

### [Item 270]

A method of predicting efficacy of an SWI/SNF complex inhibitor on a subject, comprising at least one selected from the group consisting of detecting a mutation in a CBP/P300 gene of a cancer cell of the subject, and measuring expression of a CBP/P300 protein.

### [Item 271]

The method of item 270, wherein the at least one selected from the group consisting of detecting a mutation in a CBP/P300 gene of a cancer cell, and measuring expression of a CBP/P300 protein is determined by steps comprising
(1) at least one selected from the group consisting of a step of detecting a mutation in a CBP/P300 gene of a cancer cell obtained from the subject and a step of measuring expression of a CBP/P300 gene, and
(2) a step of determining that the subject comprises at least one selected from the group consisting of a deficiency of a CBP/P300 gene, and lack of or attenuation of expression of a CBP/P300 gene based on at least one selected from the group consisting of the presence/absence of a mutation in a CBP/P300 gene and a result of expression of a CBP/P300 protein detected in (1).

### [Item 272]

The method of item 270 or 271, wherein the cancer is CBP/P300 deficient cancer.

### [Item 273]

The method of item 272, wherein the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

### [Item 274]

The method of any one of items 270 to 273, wherein the SWI/SNF complex inhibitor is a BAF complex inhibitor.

### [Item 275]

The method of item 274, wherein the BAF complex inhibitor comprises at least one inhibitor selected from the group consisting of an SMARC inhibitor and an ARID inhibitor.

### [Item 276]

The method of item 274, wherein the BAF complex inhibitor is an SMARC inhibitor.

### [Item 277]

The method of item 276, wherein the SMARC inhibitor comprises at least one inhibitor selected from the group consisting of an SMARCB1 inhibitor, an SMARCA2 inhibitor, an SMARCA4 inhibitor, and an SMARCA2/A4 inhibitor.

### [Item 278]

The method of item 276, wherein the SMARC inhibitor is an SMARCB1 inhibitor.

### [Item 279]

The method of item 278, wherein the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

### [Item 280]

The method of item 278, wherein the SMARCB1 inhibitor is a low molecular weight compound that inhibits a function of SMARCB1.

### [Item 281]

The method of item 276, wherein the SMARC inhibitor is an SMARCA2 inhibitor.

### [Item 282]

The method of item 281, wherein the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

### [Item 283]

The method of item 281, wherein the SMARCA2 inhibitor is a low molecular weight compound that inhibits a function of SMARCA2.

### [Item 284]

The method of item 276, wherein the SMARC inhibitor is an SMARCA4 inhibitor.

### [Item 285]

The method of item 284, wherein the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

### [Item 286]

The method of item 284, wherein the SMARCA4 inhibitor is a low molecular weight compound that inhibits a function of SMARCA4.

### [Item 287]

The method of item 276, wherein the SMARC inhibitor is an SMARCA2/A4 inhibitor.

### [Item 288]

The method of item 287, wherein the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

### [Item 289]

The method of item 287, wherein the SMARCA2/4 inhibitor is a low molecular weight compound that inhibits a function of SMARCA2 and SMARCA4.

### [Item 290]

The method of item 274, wherein the BAF complex inhibitor is an ARID inhibitor.

### [Item 291]

The method of item 290, wherein the ARID inhibitor is at least one inhibitor selected from the group consisting of an ARID1A inhibitor, an ARID1B inhibitor, and an ARID1A/1B inhibitor.

### [Item 292]

The method of item 290, wherein the ARID inhibitor is an ARID1A inhibitor.

### [Item 293]

The method of item 292, wherein the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

### [Item 294]

The method of item 292, wherein the ARID1A inhibitor is a low molecular weight compound that inhibits a function of ARID1A.

### [Item 295]

The method of item 290, wherein the ARID inhibitor is an ARID1B inhibitor.

### [Item 296]

The method of item 295, wherein the ARID1B inhibitor comprises at least one selected from the group consisting of a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, and precursors thereof.

### [Item 297]

The method of item 295, wherein the ARID1B inhibitor is a low molecular weight compound that inhibits a function of ARID1B.

### [Item 298]

The method of item 290, wherein the ARID inhibitor is an ARID1A/1B inhibitor.

### [Item 299]

The method of item 298, wherein the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

### [Item 300]

The method of item 298, wherein the ARID1A/1B inhibitor is a low molecular weight compound that inhibits a function of ARID1A and ARID1B.

### [Item A1]

A CBP/P300 inhibitor for use in the treatment and/or prevention of cancer.

### [Item A2]

The CBP/P300 inhibitor of item A1, wherein the cancer is SWI/SNF complex dysfunction cancer.

### [Item A3]

The CBP/P300 inhibitor of item A1, wherein the cancer is BAF complex dysfunction cancer.

### [Item A4]

The CBP/P300 inhibitor of item A1, wherein the cancer comprises at least one selected from the group consisting of SMARC deficient cancer, SS18-SSX fusion cancer, and ARID deficient cancer.

### [Item A5]

The CBP/P300 inhibitor of item A1, wherein the cancer is SMARC deficient cancer.

### [Item A6]

The CBP/P300 inhibitor of item A5, wherein the SMARC deficient cancer is cancer deficient of at least one agent selected from the group consisting of SMARCB1, SMARCA2, and SMARCA4.

### [Item A7]

The CBP/P300 inhibitor of item A5, wherein the SMARC deficient cancer comprises at least one selected from the group consisting of SMARCB1 deficient cancer, SMARCA2 deficient cancer, SMARCA4 deficient cancer, and SMARCA2/A4 deficient cancer.

### [Item A8]

The CBP/P300 inhibitor of item A5, wherein the SMARC deficient cancer is SMARCB1 deficient cancer.

### [Item A9]

The CBP/P300 inhibitor of item A8, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

### [Item A10]

The CBP/P300 inhibitor of item A8, wherein the SMARCB1 deficient cancer is malignant rhabdoid tumor, epithelioid sarcoma, or atypical teratoid/rhabdoid tumor.

### [Item A11]

The CBP/P300 inhibitor of item A8, wherein the SMARCB1 deficient cancer is malignant rhabdoid tumor.

### [Item A12]

The CBP/P300 inhibitor of item A5, wherein the SMARC deficient cancer is SMARCA2 deficient cancer.

### [Item A13]

The CBP/P300 inhibitor of item A12, wherein the SMARCA2 deficient cancer is pulmonary adenocarcinoma, large cell lung carcinoma, lung neuroendocrine tumor, esophageal cancer, gastroesophageal junction cancer, or malignant rhabdoid tumor.

### [Item A14]

The CBP/P300 inhibitor of item A12, wherein the SMARCA2 deficient cancer is pulmonary adenocarcinoma.

### [Item A15]

The CBP/P300 inhibitor of item A5, wherein the SMARC deficient cancer is SMARCA4 deficient cancer.

### [Item A16]

The CBP/P300 inhibitor of item A15, wherein the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

### [Item A17]

The CBP/P300 inhibitor of item A15, wherein the SMARCA4 deficient cancer is pulmonary adenocarcinoma.

### [Item A18]

The CBP/P300 inhibitor of item A5, wherein the SMARC deficient cancer is SMARCA2/A4 deficient cancer.

### [Item A19]

The CBP/P300 inhibitor of item A18, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

### [Item A20]

The CBP/P300 inhibitor of item A18, wherein the SMARCA2/A4 deficient cancer is pulmonary adenocarcinoma.

### [Item A21]

The CBP/P300 inhibitor of item A1, wherein the cancer is ARID deficient cancer.

### [Item A22]

The CBP/P300 inhibitor of item A21, wherein the ARID deficient cancer is cancer deficient of at least one agent selected from the group consisting of ARID1A and ARID1B.

### [Item A23]

The CBP/P300 inhibitor of item A21, wherein the ARID deficient cancer is ARID1A deficient cancer, ARID1B deficient cancer, or ARID1A/1B deficient cancer.

### [Item A24]

The CBP/P300 inhibitor of item A21, wherein the ARID deficient cancer is ARID1A deficient cancer.

### [Item A25]

The CBP/P300 inhibitor of item A24, wherein the ARID1A deficient cancer is ovarian cancer, gastric cancer, bile duct cancer, pancreatic cancer, uterine cancer, neuroblastoma, colon cancer, or bladder cancer.

### [Item A26]

The CBP/P300 inhibitor of item A25, wherein the ARID1A deficient cancer is ovarian cancer.

### [Item A27]

The CBP/P300 inhibitor of item A21, wherein the ARID deficient cancer is ARID1B deficient cancer.

### [Item A28]

The CBP/P300 inhibitor of item A27, wherein the ARID1B deficient cancer comprises at least one selected from the group consisting of ovarian cancer, colon cancer, pancreatic cancer, liver cancer, melanoma, breast cancer, medulloblastoma, uterine cancer, bladder cancer, and gastric cancer.

### [Item A29]

The CBP/P300 inhibitor of item A27, wherein the ARID1B deficient cancer is ovarian cancer.

### [Item A30]

The CBP/P300 inhibitor of item A21, wherein the ARID deficient cancer is ARID1A/1B deficient cancer.

### [Item A31]

The CBP/P300 inhibitor of item A30, wherein the ARID1A/1B deficient cancer comprises at least one selected from the group consisting of ovarian cancer, colon cancer, uterine cancer, neuroblastoma, bladder cancer, and gastric cancer.

### [Item A32]

The CBP/P300 inhibitor of item A30, wherein the ARID1A/1B deficient cancer is ovarian cancer.

### [Item A33]

The CBP/P300 inhibitor of item A1, wherein the cancer is SS18-SSX fusion cancer.

### [Item A34]

The CBP/P300 inhibitor of item A33, wherein the SS18-SSX fusion cancer is synovial sarcoma or Ewing's sarcoma.

### [Item A35]

The CBP/P300 inhibitor of item A33, wherein the SS18-SSX fusion cancer is synovial sarcoma.

### [Item A36]

The CBP/P300 inhibitor of any one of items A1 to A35, wherein the CBP/P300 inhibitor is a HAT inhibitor, a BRD inhibitor, an antisense nucleic acid for a transcriptional product of a gene encoding CBP or P300, a ribozyme for a transcriptional product of a gene encoding CBP or P300, a nucleic acid having RNAi activity for a transcriptional product of a gene encoding CBP or P300, or a precursor thereof.

### [Item A37]

The CBP/P300 inhibitor of any one of items A1 to A36, wherein the CBP/P300 inhibitor is a HAT inhibitor or a BRD inhibitor.

### [Item A38]

The CBP/P300 inhibitor of any one of items A1 to A37, wherein the CBP/P300 inhibitor is a HAT inhibitor.

### [Item A39]

The CBP/P300 inhibitor of any one of items A36 to A38, wherein activity of the HAT inhibitor inhibits histone acetyltransferase (HAT) activity of CBP and/or P300 by 50% or more at 20 μM.

### [Item A40]

The CBP/P300 inhibitor of any one of items A36 to A38, wherein activity of the HAT inhibitor inhibits histone acetyltransferase (HAT) activity of CBP and/or P300 by 80% or more at 20 μM.

### [Item A41]

The CBP/P300 inhibitor of any one of items A1 to A40, wherein the CBP/P300 inhibitor is a nucleic acid or a low molecular weight compound.

### [Item A42]

The CBP/P300 inhibitor of any one of items A36 to A41, wherein the HAT inhibitor is a low molecular weight compound.

### [Item A43]

An SWI/SNF complex inhibitor for use in the treatment and/or prevention of cancer.

### [Item A44]

The SWI/SNF complex inhibitor of item A43, wherein the cancer is CBP/P300 deficient cancer.

### [Item A45]

The SWI/SNF complex inhibitor of item A44, wherein the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

### [Item A46]

The SWI/SNF complex inhibitor of any one of items A43 to A45, wherein the SWI/SNF complex inhibitor is a BAF complex inhibitor.

### [Item A47]

The BAF complex inhibitor of item A46, wherein the BAF complex inhibitor is at least one inhibitor selected from the group consisting of an SMARC inhibitor and an ARID inhibitor.

### [Item A48]

The BAF complex inhibitor of item A46 or A47, wherein the BAF complex inhibitor is an SMARC inhibitor.

### [Item A49]

The SMARC inhibitor of item A47 or A48, wherein the SMARC inhibitor is at least one inhibitor selected from the group consisting of an SMARCB1 inhibitor, an SMARCA2 inhibitor, an SMARCA4 inhibitor, and an SMARCA2/A4 inhibitor.

### [Item A50]

The SMARC inhibitor of item A47 or A48, wherein the SMARC inhibitor is an SMARCB1 inhibitor.

### [Item A51]

The SMARC inhibitor of item A50, wherein the SMARCB1 inhibitor is a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, or a precursor thereof.

### [Item A52]

The SMARC inhibitor of item A50, wherein SMARCB1 inhibitor is a low molecular weight compound that inhibits a function of SMARCB1.

### [Item A53]

The SMARC inhibitor of item A47 or A48, wherein the SMARC inhibitor is an SMARCA2 inhibitor.

### [Item A54]

The SMARC inhibitor of item A53, wherein the SMARCA2 inhibitor is a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, or a precursor thereof.

### [Item A55]

The SMARC inhibitor of item A53, wherein the SMARCA2 inhibitor is a low molecular weight compound that inhibits a function of SMARCA2.

### [Item A56]

The SMARC inhibitor of item A47 or A48, wherein the SMARC inhibitor is an SMARCA4 inhibitor.

### [Item A57]

The SMARC inhibitor of item A56, wherein the SMARCA4 inhibitor is a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, or a precursor thereof.

### [Item A58]

The SMARC inhibitor of item A56, wherein SMARCA4 inhibitor is a low molecular weight compound that inhibits a function of SMARCA4.

### [Item A59]

The SMARC inhibitor of item A47 or A48, wherein the SMARC inhibitor is an SMARCA2/A4 inhibitor.

### [Item A60]

The SMARC inhibitor of item A59, wherein the SMARCA2/4 inhibitor is a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, or a precursor thereof.

### [Item A61]

The SMARC inhibitor of item A59, wherein the SMARCA2/4 inhibitor is a low molecular weight compound that inhibits a function of SMARCA2 and SMARCA4.

### [Item A62]

The SWI/SNF complex inhibitor of item A46, wherein the BAF complex inhibitor is an ARID inhibitor.

### [Item A63]

The SWI/SNF complex inhibitor of item A62, wherein the ARID inhibitor is at least one inhibitor selected from the group consisting of an ARID1A inhibitor, an ARID1B inhibitor, and an ARID1A/1B inhibitor.

### [Item A64]

The SWI/SNF complex inhibitor of item A62 or A63, wherein the ARID inhibitor is an ARID1A inhibitor.

### [Item A65]

The SWI/SNF complex inhibitor of item A65, wherein the ARID1A inhibitor is a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, or a precursor thereof.

### [Item A66]

The SWI/SNF complex inhibitor of item A65, wherein ARID1A inhibitor is a low molecular weight compound that inhibits a function of ARID1A.

### [Item A67]

The SWI/SNF complex inhibitor of item A62 or A63, wherein the ARID inhibitor is an ARID1B inhibitor.

### [Item A68]

The SWI/SNF complex inhibitor of item A67, wherein the ARID1B inhibitor is a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, or a precursor thereof.

### [Item A69]

The SWI/SNF complex inhibitor of item A67, wherein the ARID1B inhibitor is a low molecular weight compound that inhibits a function of ARID1B.

### [Item A70]

The SWI/SNF complex inhibitor of item A62 or A63, wherein the ARID inhibitor is an ARID1A/1B inhibitor.

### [Item A71]

The SWI/SNF complex inhibitor of item A70, wherein the ARID1A/1B inhibitor is a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, or a precursor thereof.

### [Item A72]

The SWI/SNF complex inhibitor of item A70, wherein ARID1A/1B inhibitor is a low molecular weight compound that inhibits a function of ARID1A and ARID1B.

### [Item B1]

A method of treating and/or preventing cancer in a subject, comprising the step of administering an effective amount of a CBP/P300 inhibitor to the subject.

### [Item B2]

The method of item B1, wherein the cancer is SWI/SNF complex dysfunction cancer.

### [Item B3]

The method of item B1, wherein the cancer is BAF complex dysfunction cancer.

### [Item B4]

The method of item B1, wherein the cancer is SMARC deficient cancer, SS18-SSX fusion cancer, or ARID deficient cancer.

### [Item B5]

The method of item B1, wherein the cancer is SMARC deficient cancer.

### [Item B6]

The method of item B5, wherein the SMARC deficient cancer is cancer deficient of at least one agent selected from the group consisting of SMARCB1, SMARCA2, and SMARCA4.

### [Item B7]

The method of item B5, wherein the SMARC deficient cancer is SMARCB1 deficient cancer, SMARCA2 deficient cancer, SMARCA4 deficient cancer, or SMARCA2/A4 deficient cancer.

### [Item B8]

The method of item B5, wherein the SMARC deficient cancer is SMARCB1 deficient cancer.

### [Item B9]

The method of item B8, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

### [Item B10]

The method of item B8, wherein the SMARCB1 deficient cancer is malignant rhabdoid tumor, epithelioid sarcoma, or atypical teratoid/rhabdoid tumor.

### [Item B11]

The method of item B8, wherein the SMARCB1 deficient cancer is malignant rhabdoid tumor.

### [Item B12]

The method of item B5, wherein the SMARC deficient cancer is SMARCA2 deficient cancer.

### [Item B13]

The method of item B12, wherein the SMARCA2 deficient cancer comprises at least one selected from the group consisting of pulmonary adenocarcinoma, large cell lung carcinoma, lung neuroendocrine tumor, esophageal cancer, gastroesophageal junction cancer, and malignant rhabdoid tumor.

### [Item B14]

The method of item B12, wherein the SMARCA2 deficient cancer is pulmonary adenocarcinoma.

### [Item B15]

The method of item B5, wherein the SMARC deficient cancer is SMARCA4 deficient cancer.

### [Item B16]

The method of item B15, wherein the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

### [Item B17]

The method of item B15, wherein the SMARCA4 deficient cancer is pulmonary adenocarcinoma.

### [Item B18]

The method of item B5, wherein the SMARC deficient cancer is SMARCA2/A4 deficient cancer.

### [Item B19]

The method of item B18, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

### [Item B20]

The method of item B18, wherein the SMARCA2/A4 deficient cancer is pulmonary adenocarcinoma.

### [Item B21]

The method of item B1, wherein the cancer is ARID deficient cancer.

### [Item B22]

The method of item B21, wherein the ARID deficient cancer is cancer deficient of at least one agent selected from the group consisting of ARID1A and ARID1B.

### [Item B23]

The method of item B21, wherein the ARID deficient cancer is ARID1A deficient cancer, ARID1B deficient cancer, or ARID1A/1B deficient cancer.

### [Item B24]

The method of item B21, wherein the ARID deficient cancer is ARID1A deficient cancer.

### [Item B25]

The method of item B24, wherein the ARID1A deficient cancer comprises at least one selected from the group consisting of ovarian cancer, gastric cancer, bile duct cancer, pancreatic cancer, uterine cancer, neuroblastoma, colon cancer, and bladder cancer.

### [Item B26]

The method of item B24, wherein the ARID1A deficient cancer is ovarian cancer.

### [Item B27]

The method of item B21, wherein the ARID deficient cancer is ARID1B deficient cancer.

### [Item B28]

The method of item B27, wherein the ARID1B deficient cancer comprises at least one selected from the group consisting of ovarian cancer, colon cancer, pancreatic cancer, liver cancer, melanoma, breast cancer, medulloblastoma, uterine cancer, bladder cancer, and gastric cancer.

### [Item B29]

The method of item B27, wherein the ARID1B deficient cancer is ovarian cancer.

### [Item B30]

The method of item B21, wherein the ARID deficient cancer is ARID1A/1B deficient cancer.

### [Item B31]

The method of item B30, wherein the ARID1A/1B deficient cancer is ovarian cancer, colon cancer, uterine cancer, neuroblastoma, bladder cancer, or gastric cancer.

### [Item B32]

The method of item B30, wherein the ARID1A/1B deficient cancer is ovarian cancer.

### [Item B33]

The method of item B1, wherein the cancer is SS18-SSX fusion cancer.

### [Item B34]

The method of item B33, wherein the SS18-SSX fusion cancer is synovial sarcoma or Ewing's sarcoma.

### [Item B35]

The method of item B33, wherein the SS18-SSX fusion cancer is synovial sarcoma.

### [Item B36]

The method of any one of items B1 to B35, wherein the CBP/P300 inhibitor comprises at least one selected from the group consisting of a HAT inhibitor, a BRD inhibitor, an antisense nucleic acid for a transcriptional product of a gene encoding CBP or P300, a ribozyme for a transcriptional product of a gene encoding CBP or P300, a nucleic acid having RNAi activity for a transcriptional product of a gene encoding CBP or P300, and precursors thereof.

### [Item B37]

The method of any one of items B1 to B36, wherein the CBP/P300 inhibitor is a HAT inhibitor or a BRD inhibitor.

### [Item B38]

The method of any one of items B1 to B37, wherein the CBP/P300 inhibitor is a HAT inhibitor.

### [Item B39]

The method of any one of items B36 to B38, wherein activity of the HAT inhibitor inhibits histone acetyltransferase (HAT) activity of CBP and/or P300 by 50% or more at 20 μM.

### [Item B40]

The method of any one of items B36 to B39, wherein activity of the HAT inhibitor inhibits histone acetyltransferase (HAT) activity of CBP and/or P300 by 80% or more at 20 μM.

### [Item B41]

The method of any one of items B36 to B40, wherein the HAT inhibitor is a nucleic acid or a low molecular weight compound.

### [Item B42]

The method of any one of items B36 to B41, wherein the HAT inhibitor is a low molecular weight compound.

### [Item B43]

A method of treating and/or preventing cancer in a subject, comprising the step of administering an effective amount of an SWI/SNF complex inhibitor to the subject.

### [Item B44]

The method of item B43, wherein the cancer is CBP/P300 deficient cancer.

### [Item B45]

The method of item B44, wherein the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

### [Item B46]

The method of any one of items B43 to B45, wherein the SWI/SNF complex inhibitor is a BAF complex inhibitor.

### [Item B47]

The method of item B46, wherein the BAF complex inhibitor is at least one inhibitor selected from the group consisting of an SMARC inhibitor and an ARID inhibitor.

### [Item B48]

The method of item B46, wherein the BAF complex inhibitor is an SMARC inhibitor.

### [Item B49]

The method of item B47 or B48, wherein the SMARC inhibitor is at least one inhibitor selected from the group consisting of an SMARCB1 inhibitor, an SMARCA2 inhibitor, an SMARCA4 inhibitor, and an SMARCA2/A4 inhibitor.

### [Item B50]

The method of any one of items B47 to B49, wherein the SMARC inhibitor is an SMARCB1 inhibitor.

### [Item B51]

The method of item B50, wherein the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

### [Item B52]

The method of item B50, wherein the SMARCB1 inhibitor is a low molecular weight compound.

### [Item B53]

The method of any one of items B47 to B49, wherein the SMARC inhibitor is an SMARCA2 inhibitor.

### [Item B54]

The method of item B53, wherein the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

### [Item B55]

The method of item B53, wherein the SMARCA2 inhibitor is a low molecular weight compound.

### [Item B56]

The method of any one of items B47 to B49, wherein the SMARC inhibitor is an SMARCA4 inhibitor.

### [Item B57]

The method of item B56, wherein the SMARCA4 inhibitor is a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, or a precursor thereof.

### [Item B58]

The method of item B56, wherein the SMARCA4 inhibitor is a low molecular weight compound.

### [Item B59]

The method of any one of items B47 to B49, wherein the SMARC inhibitor is an SMARCA2/A4 inhibitor.

### [Item B60]

The method of item B59, wherein the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

### [Item B61]

The method of item B59, wherein the SMARCA2/A4 inhibitor is a low molecular weight compound that inhibits a function of SMARCA2 and SMARCA4.

### [Item B62]

The method of item B46, wherein the BAF complex inhibitor is an ARID inhibitor.

### [Item B63]

The method of item B62, wherein the ARID inhibitor is at least one inhibitor selected from the group consisting of an ARID1A inhibitor, an ARID1B inhibitor, and an ARID1A/1B inhibitor.

### [Item B64]

The method of item B62, wherein the ARID inhibitor is an ARID1A inhibitor.

### [Item B65]

The method of item B64, wherein the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

### [Item B66]

The method of item B64, wherein the ARID1A inhibitor is a low molecular weight compound.

### [Item B67]

The method of item B62 or B63, wherein the ARID inhibitor is an ARID1B inhibitor.

### [Item B68]

The method of item B67, wherein the ARID1B inhibitor is a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, or a precursor thereof.

### [Item B69]

The method of item B67, wherein the ARID1B inhibitor is a low molecular weight compound.

### [Item B70]

The method of item B62 or B63, wherein the ARID inhibitor is an ARID1A/1B inhibitor.

### [Item B71]

The method of item B70, wherein the ARID1A/1B inhibitor is a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, or a precursor thereof.

### [Item B72]

The method of item B70, wherein the ARID1A/1B inhibitor is a low molecular weight compound that inhibits a function of ARID1A and ARID1B.

### [Item C1]

Use of a CBP/P300 inhibitor in the manufacture of a medicament for use in treating and/or preventing cancer.

### [Item C2]

The use of item C1, wherein the cancer is SWI/SNF complex dysfunction cancer.

### [Item C3]

The use of item C1, wherein the cancer is BAF complex dysfunction cancer.

### [Item C4]

The use of item C1, wherein the cancer is SMARC deficient cancer, SS18-SSX fusion cancer, or ARID deficient cancer.

### [Item C5]

The use of item C1, wherein the cancer is SMARC deficient cancer.

### [Item C6]

The use of item C5, wherein the SMARC deficient cancer is cancer deficient of at least one agent selected from the group consisting of SMARCB1, SMARCA2, and SMARCA4.

### [Item C7]

The use of item C5, wherein the SMARC deficient cancer is SMARCB1 deficient cancer, SMARCA2 deficient cancer, SMARCA4 deficient cancer, or SMARCA2/A4 deficient cancer.

### [Item C8]

The use of item C5, wherein the SMARC deficient cancer is SMARCB1 deficient cancer.

### [Item C9]

The use of item C8, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

### [Item C10]

The use of item C8, wherein the SMARCB1 deficient cancer comprises at least one selected from the group consisting of malignant rhabdoid tumor, epithelioid sarcoma, and atypical teratoid/rhabdoid tumor.

### [Item C11]

The use of item C8, wherein the SMARCB1 deficient cancer is malignant rhabdoid tumor.

### [Item C12]

The use of item C5, wherein the SMARC deficient cancer is SMARCA2 deficient cancer.

### [Item C13]

The use of item C12, wherein the SMARCA2 deficient cancer comprises at least one selected from the group consisting of pulmonary adenocarcinoma, large cell lung carcinoma, lung neuroendocrine tumor, esophageal cancer, gastroesophageal junction cancer, and malignant rhabdoid tumor.

### [Item C14]

The use of item C12, wherein the SMARCA2 deficient cancer is pulmonary adenocarcinoma.

### [Item C15]

The use of item C5, wherein the SMARC deficient cancer is SMARCA4 deficient cancer.

### [Item C16]

The use of item C15, wherein the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

### [Item C17]

The use of item C15, wherein the SMARCA4 deficient cancer is pulmonary adenocarcinoma.

### [Item C18]

The use of item C5, wherein the SMARC deficient cancer is SMARCA2/A4 deficient cancer.

### [Item C19]

The use of item C18, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

### [Item C20]

The use of item C18, wherein the SMARCA2/A4 deficient cancer is pulmonary adenocarcinoma.

### [Item C21]

The use of item C1, wherein the cancer is ARID deficient cancer.

### [Item C22]

The use of item C21, wherein the ARID deficient cancer is cancer deficient of at least one agent selected from the group consisting of ARID1A and ARID1B.

### [Item C23]

The use of item C21, wherein the ARID deficient cancer is ARID1A deficient cancer, ARID1B deficient cancer, or ARID1A/1B deficient cancer.

### [Item C24]

The use of item C21, wherein the ARID deficient cancer is ARID1A deficient cancer.

### [Item C25]

The use of item C24, wherein the ARID1A deficient cancer is ovarian cancer, gastric cancer, bile duct cancer, pancreatic cancer, uterine cancer, neuroblastoma, colon cancer, or bladder cancer.

### [Item C26]

The use of item C24, wherein the ARID1A deficient cancer is ovarian cancer.

### [Item C27]

The use of item C21, wherein the ARID deficient cancer is ARID1B deficient cancer.

### [Item C28]

The use of item C27, wherein the ARID1B deficient cancer is ovarian cancer, colon cancer, pancreatic cancer, liver cancer, melanoma, breast cancer, medulloblastoma, uterine cancer, bladder cancer, or gastric cancer.

### [Item C29]

The use of item C27, wherein the ARID1B deficient cancer is ovarian cancer.

### [Item C30]

The use of item C21, wherein the ARID deficient cancer is ARID1A/1B deficient cancer.

### [Item C31]

The use of item C30, wherein the ARID1A/1B deficient cancer is ovarian cancer, colon cancer, uterine cancer, neuroblastoma, bladder cancer, or gastric cancer.

### [Item C32]

The use of item C30, wherein the ARID1A/1B deficient cancer is ovarian cancer.

### [Item C33]

The use of item C1, wherein the cancer is SS18-SSX fusion cancer.

### [Item C34]

The use of item C33, wherein the SS18-SSX fusion cancer is synovial sarcoma or Ewing's sarcoma.

### [Item C35]

The use of item C33, wherein the SS18-SSX fusion cancer is synovial sarcoma.

### [Item C36]

The use of any one of items C1 to C35, wherein the CBP/P300 inhibitor is a HAT inhibitor, a BRD inhibitor, an antisense nucleic acid for a transcriptional product of a gene encoding CBP or P300, a ribozyme for a transcriptional product of a gene encoding CBP or P300, a nucleic acid having RNAi activity for a transcriptional product of a gene encoding CBP or P300, or a precursor thereof.

### [Item C37]

The use of any one of items C1 to C36, wherein the CBP/P300 inhibitor is a HAT inhibitor or a BRD inhibitor.

### [Item C38]

The use of any one of items C1 to C36, wherein the CBP/P300 inhibitor is a HAT inhibitor.

### [Item C39]

The use of any one of items C36 to C38, wherein activity of the HAT inhibitor inhibits histone acetyltransferase (HAT) activity of CBP and/or P300 by 50% or more at 20 μM.

### [Item C40]

The use of any one of items C36 to C39, wherein activity of the HAT inhibitor inhibits histone acetyltransferase (HAT) activity of CBP and/or P300 by 80% or more at 20 μM.

### [Item C41]

The use of any one of items C36 to C40, wherein the HAT inhibitor is a nucleic acid or a low molecular weight compound.

### [Item C42]

The use of any one of items C36 to C41, wherein the HAT inhibitor is a low molecular weight compound.

### [Item C43]

Use of an SWI/SNF inhibitor in the manufacture of a medicament for use in treating and/or preventing cancer.

### [Item C44]

The use of item C43, wherein the cancer is CBP/P300 deficient cancer.

### [Item C45]

The use of item C44, wherein the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

### [Item C46]

The use of any one of items C43 to C45, wherein the SWI/SNF complex inhibitor is a BAF complex inhibitor.

### [Item C47]

The use of item C46, wherein the BAF complex inhibitor is at least one inhibitor selected from the group consisting of an SMARC inhibitor and an ARID inhibitor.

### [Item C48]

The use of item C46 or C47, wherein the BAF complex inhibitor is an SMARC inhibitor.

### [Item C49]

The use of item C47 or C48, wherein the SMARC inhibitor is at least one inhibitor selected from the group consisting of an SMARCB1 inhibitor, an SMARCA2 inhibitor, an SMARCA4 inhibitor, and an SMARCA2/A4 inhibitor.

### [Item C50]

The use of item C47 or C48, wherein the SMARC inhibitor is an SMARCB1 inhibitor.

### [Item C51]

The use of item C50, wherein the SMARCB1 inhibitor is a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, or a precursor thereof.

### [Item C52]

The use of item C50, wherein the SMARCB1 inhibitor is a low molecular weight compound.

### [Item C53]

The use of item C47 or C48, wherein the SMARC inhibitor is an SMARCA2 inhibitor.

### [Item C54]

The use of item C53, wherein the SMARCA2 inhibitor is a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, or a precursor thereof.

### [Item C55]

The use of item C53, wherein the SMARCA2 inhibitor is a low molecular weight compound.

### [Item C56]

The use of item C47 or C48, wherein the SMARC inhibitor is an SMARCA4 inhibitor.

### [Item C57]

The use of item C56, wherein the SMARCA4 inhibitor is a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, or a precursor thereof.

### [Item C58]

The use of item C56, wherein the SMARCA4 inhibitor is a low molecular weight compound.

### [Item C59]

The use of item C47 or C48, wherein the SMARC inhibitor is an SMARCA2/A4 inhibitor.

### [Item C60]

The use of item C59, wherein the SMARCA2/4 inhibitor is a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, or a precursor thereof.

### [Item C61]

The use of item C59, wherein the SMARCA2/4 inhibitor is a low molecular weight compound that inhibits a function of SMARCA2 and SMARCA4.

### [Item C62]

The use of item C46, wherein the BAF complex inhibitor is an ARID inhibitor.

### [Item C63]

The use of item C62, wherein the ARID inhibitor is at least one inhibitor selected from the group consisting of an ARID1A inhibitor, an ARID1B inhibitor, and an ARID1A/1B inhibitor.

### [Item C64]

The use of item C62, wherein the ARID inhibitor is an ARID1A inhibitor.

### [Item C65]

The use of item C64, wherein the ARID1A inhibitor is a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, or a precursor thereof.

### [Item C66]

The use of item C64, wherein the ARID1A inhibitor is a low molecular weight compound.

### [Item C67]

The use of item C62, wherein the ARID inhibitor is an ARID1B inhibitor.

### [Item C68]

The use of item C67, wherein the ARID1B inhibitor is a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, or a precursor thereof.

### [Item C69]

The use of item C62, wherein the ARID1B inhibitor is a low molecular weight compound.

### [Item C70]

The use of item C62, wherein the ARID inhibitor is an ARID1A/1B inhibitor.

### [Item C71]

The use of item C70, wherein the ARID1A/1B inhibitor is a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, or a precursor thereof.

### [Item C72]

The use of item C70, wherein the ARID1A/1B inhibitor is a low molecular weight compound.

### Advantageous Effects of Invention

The CBP/P300 inhibitor of the present disclosure is effective for use in treating and/or preventing SWI/SNF complex dysfunction cancer.

### BRIEF DESCRIPTION OF DRAWINGS

FIG. 1 is a diagram from detecting the SMARCB1 protein expression level in JMU-RTK-2 cells, which are SMARCB1 deficient cells, and JMU-RTK-2+SMARCB1, which are JMU-RTK-2 cells overexpressing SMARCB1, by Western blot. The β-Actin protein level is shown as a loading control.

FIG. 2 is a diagram showing cytotoxic activity of compound 4 on JMU-RTK-2 cells, which are SMARCB1 deficient cells, and JMU-RTK-2+SMARCB1 cells, which are JMU-RTK-2 cells overexpressing SMARCB1. The vertical axis represents the cell viability (% with respect to negative control group added with a medium DMSO). The horizontal axis represents the treatment concentration (μM) of the compound. Black dots indicate results for JMU-RTK-2 cells. Squares indicate results for JMU-RTK-2+SMARCB1 cells.

FIG. 3 is a diagram comparing cytotoxic activity of compound 16 or a BRD inhibitor SGC-CBP30 on SMARCB1 deficient cells, i.e., G-401 cells, G-402 cells, JMU-RTK-2 cells, and HS-ES-1 cells, and SMARCB1 wild-type cells, i.e., 786-O cells, VMRC-RCZ cells, Caki-1 cells, H446 cells, ES2 cells, H460 cells, H2228 cells, HEK293T cells, and H358 cells. The vertical axis represents the IC₅₀ values (μM) of each compound. Black dots indicate the IC₅₀ values for individual SMARCB1 wild-type cells. Squares indicate the IC₅₀ values for individual SMARCB1 deficient cells. Bar graphs indicate the mean value±standard error of IC₅₀ values for each group.

FIG. 4 is a diagram showing the expression level of mRNA of each gene when expression of gene CREBBP encoding CBP and/or gene EP300 encoding P300 was suppressed with siRNA in SMARCB1 deficient cells, i.e., G-402 cells, JMU-RTK-2 cells, and HS-ES-1 cells, SMARCB1 wild-type cells, i.e., 786-O cells and VMRC-RCZ cells, and JMU-RTK-2+SMARCB1 cells, which are JMU-RTK-2 cells overexpressing SMARCB1. The vertical axis represents the relative mRNA expression level. Data is indicated as mean value±standard deviation. siNT indicates the negative control of siRNA.

FIG. 5 is a diagram showing the cell viability rate when expression of gene CREBBP encoding CBP and/or gene EP300 encoding P300 was suppressed with siRNA in SMARCB1 deficient cells, i.e., G-402 cells, JMU-RTK-2 cells, and HS-ES-1 cells, SMARCB1 wild-type cells, i.e., 786-0 cells and VMRC-RCZ cells, and JMU-RTK-2+SMARCB1 cells, which are JMU-RTK-2 cells overexpressing SMARCB1. The vertical axis represents the cell viability rate (%) for the negative control group siNT. Data is indicated as mean value±standard deviation.

FIG. 6 is a diagram showing the ability to form a colony when expression of gene CREBBP encoding CBP and/or gene EP300 encoding P300 was suppressed with siRNA in SMARCB1 deficient cells, i.e., G-402 cells and JMU-RTK-2 cells, SMARCB1 wild-type cells, i.e., 786-O cells and VMRC-RCZ cells. siNT indicates the negative control of siRNA.

FIG. 7 is a diagram from detecting the histone H3K27 acetylation level in G-401 cells and CHLA-06-ATRT cells for compounds 1 to 16 by Western blot. β-Actin protein level is shown as a loading control.

FIG. 8 is a diagram comparing cytotoxic activity of compounds 4, 16, and BRD inhibitor CCS-1477 on SMARCA2/A4 deficient cells, i.e., H23 cells, A427 cells, SW13 cells, COV434 cells, DMS114 cells, and TOV112D cells, and SMARCA2/A4 wild-type cells, i.e., H1048 cells, H460 cells, 786-O cells, H2228 cells, H2009 cells, and H358 cells. The vertical axis represents the IC50 value (μM) of each compound. Black dots indicate the IC50 values of individual SMARCA2/A4 wild-type cells. Triangles indicate the IC50 values of individual SMARCA2/A4 deficient cells. The bar graphs indicate the mean value±standard error of IC50 values of each group.

FIG. 9 is a diagram showing the mRNA expression level of each gene when expression of gene CREBBP encoding CBP and/or gene EP300 encoding P300 was suppressed with siRNA in SMARCA2/A4 deficient cells, i.e., H23 cells and DMS114 cells, and SMARCA2/A4 wild-type cells, i.e., H460 cells. The vertical axis represents the relative mRNA expression level. Data is indicated as mean value±standard deviation. siNT indicates the negative control of siRNA.

FIG. 10 is a diagram showing the cell viability rate when expression of gene CREBBP encoding CBP and/or gene EP300 encoding P300 was suppressed with siRNA in SMARCA2/A4 deficient cells, i.e., H23 cells and DMS114 cells, and SMARCA2/A4 wild-type cells, i.e., H460 cells. The vertical axis represents the cell viability rate (%) for the negative control group siNT. Data is indicated as mean value±standard deviation.

FIG. 11 is a diagram comparing cytotoxic activity of compounds 4, 16, and BRD inhibitor CCS-1477 on SS18-SSX fusion cancer cells, i.e., Fuji cells, Aska-SS cells, Yamato-SS cells, HS-SY-II cells, and NCC-SS1-C₁ cells, and SS18/SSX wild-type cells, i.e., H1048 cells, H460 cells, 786-O cells, H2228 cells, H2009 cells, and H358 cells. The vertical axis represents the IC50 value (μM) of each compound. Black dots indicate the IC50 values of individual SS18/SSX wild-type cells. Triangles indicate the IC50 values of individual SS18-SSX fusion cancer cells. The bar graphs indicate the mean value±standard error of IC50 values of each group.

FIG. 12 is a diagram comparing cytotoxic activity of compounds 4, 16, and BRD inhibitor CCS-1477 on ARID1 deficient cancer cells, i.e., A2780 cells, RMG-V cells, TOV21G cells, and OVISE cells, and ARID1 wild-type cells, i.e., H1048 cells, H460 cells, 786-O cells, H2228 cells, H2009 cells, and H358 cells. The vertical axis represents the IC50 value (μM) of each compound. Black dots indicate the IC50 values of individual ARID1 wild-type cells. Triangles indicate the IC50 values of individual ARID1 deficient cancer cells. The bar graphs indicate the mean value±standard error of IC50 values of each group.

FIG. 13 is a diagram explaining the types of SWI/SNF complexes, each constituent, and the relationship between deficiency/fusion of each constituent and complex dysfunction. SWI/SNF complex is the general term for protein complexes comprised of a plurality of constituents regulating overall gene expression by ATP dependently changing the chromatin structure. Such complexes are roughly classified into three types of complexes (BAF complex, PBAF complex, and ncBAF complex) with different constituents. SMARCB1 is a constituent of a BAF complex and a PBAF complex. Deficiency thereof results in dysfunction of BAF complexes and PBAF complexes. SMARCA2 and SMARCA4 are constituents of all three complexes. Deficiency thereof results in dysfunction in all complexes. ARID1A and ARID1B are constituents of a BAF complex. Deficiency thereof results in dysfunction of a BAF complex. SS18 is a constituent of a BAF complex and an ncBAF complex. SS18 fuses to SSX that is inherently not a constituent of an SWI/SNF complex and pushes out SMARCB1 in the vicinity thereof from a BAF complex, resulting in BAF complex dysfunction in the same manner as SMARCB1 deficiency.

### DESCRIPTION OF EMBODIMENTS

The present disclosure is described in more detail hereinafter. Throughout the entire specification, a singular expression should be understood as encompassing the concept thereof in the plural form, unless specifically noted otherwise. Thus, singular articles (e.g., “a”, “an”, “the”, and the like in case of English) should also be understood as encompassing the concept thereof in the plural form, unless specifically noted otherwise. The terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present disclosure pertains. In case of a contradiction, the present specification (including the definitions) takes precedence.

“CBP” and “P300” herein are both histone acetyltransferases involved in the regulation of chromatin and are paralogs in relation to each other. Histone acetyltransferase is mainly, but not solely, an enzyme which transfers an acetyl group to a lysine residue that is present on the amino terminal tail of a histone protein. CBP and P300 mainly, but not solely, acetylate histone H2A, H2B, H3, or H4. In particular, histone H3 mainly, but not solely, acetylates lysine 18, lysine 27, lysine 56, and lysine 122 (H3K18, H3K27, H3K56, and H3K122, respectively) residues. In particular, acetylation of histone H3K27 is known as a marker for an open chromatin, and serves a critical role in the regulation of gene expression (J Hum Genet. 2013 July; 58(7): 439-45). p53 (Cell. 1997 August; 90(4): 595-606), MyoD (J Biol Chem. 2000 November; 275(44): 34359-34364), STAT3 (Science. 2005 January; 307 (5707): 269-273), Androgen receptor (J Biol Chem. 2000 July; 275 (27): 20853-20860), etc. have been reported as substrates other than histone. When expressed as “CBP” and “P300” herein, a protein is generally referred, but may refer to a nucleic acid encoding the same or a gene as a concept in accordance with the situation. Those skilled in the art can appropriately understand the term in accordance with the context.

Important functional domains of CBP and P300 include HAT domain, bromo domain (BRD), CH1/CH2/CH3 domain (cysteine-histidine rich domains), KIX domain, etc. (Mol Genet Metab. 2016. 119 (1-2): 37-43). HAT domain is mainly, but not solely, a domain that has activity to transfer an acetyl group to a lysine residue that is present on an amino terminal tail of a histone protein. A bromo domain is mainly, but not solely, a protein domain that recognizes an N-acetylated lysine residue found on an amino terminal tail of a histone protein.

The term “CBP” as used herein refers to any naturally-occurring CBP derived from any vertebrate source including mammals such as primates (e.g., human) and rodents (e.g., mouse and rat), unless specified otherwise. The term encompasses unprocessed CBP and any form of CBP resulting from processing in a cell. The term also encompasses naturally-occurring variants of CBP such as splice variants and allelic variants. Human CBP is registered as UniProt Accession Number: Q92793. Representative amino acid sequences of human CBP are set forth in UniProt Q92793-1 (SEQ ID NO: 1) and UniProt Q92793-2 (SEQ ID NO: 2).

The term “P300” as used herein refers to any naturally-occurring P300 derived from any vertebrate source including mammals such as primates (e.g., human) and rodents (e.g., mouse and rat), unless specified otherwise. The term encompasses unprocessed P300 and any form of P300 resulting from processing in a cell. The term also encompasses naturally-occurring variants of P300 such as splice variants and allelic variants. Human P300 is registered as UniProt Accession Number: Q09472. A representative amino acid sequence of human P300 is set forth in UniProt 009472-1 (SEQ ID NO: 3).

“CBP/P300 inhibitor” is a substance that inactivates, reduces the activity of, and/or reduces the expression of CBP and/or P300. “Reduced expression of CBP/P300” may be expression manifested at any stage such as the level prior to transcription (e.g., genome stage), transcription level, post-transcription regulation level, translation level, or post-translation modification level.

“CBP/P300 inhibitor” is preferably a HAT inhibitor or BRD inhibitor, and more preferably a HAT inhibitor.

“HAT inhibitor” is a compound that inhibits the histone acetyltransferase (HAT) activity of CBP and/or P300. For example, a method of detecting CoA-SH generated as a byproduct in a histone acetyltransferase reaction by fluorescence (e.g., Gao T. et al., Methods Mol Biol. 2013; 981: 229-38), a method of detection using a radioisotope (e.g., Lau O D et al. J Biol Chem. 2000; 275(29): 21953-9), a method of detecting acetylated histone peptide by TR-FRET (e.g., PerkinElmer, LANCE Ultra or AlphaLISA products), a method of detection using NADH (e.g., Berndsen et al., Methods. 2005; 36(4): 321-31), etc. can be utilized for the detection of histone acetyltransferase activity. Examples of HAT inhibitors include compounds disclosed in WO 2016/044770, WO 2016/044771, WO 2016/044777, WO 2018/235966, WO 2019/111980, WO 2019/049061, WO 2019/161162, WO 2019/161157, WO 2019/201291, and WO 2020/108500.

“BRD inhibitor” is a compound that inhibits the function of a bromo domain (BRD) of CBP and/or P300. For example, a method of detecting a bond between a bromo domain and an acetylated lysine residue by TR-FRET (e.g., Acta Pharmacol Sin. 2020; 41(2): 286-292), etc. can be utilized for the detection of the function of a bromo domain. Examples of BRD inhibitors include compounds disclosed in WO 2017/205538, WO 2016/086200, WO 2018/073586, WO 2019/055877, WO 2017/140728, WO 2019/191667, and WO 2019/195846.

Histone acetyltransferase (HAT) activity is enzymatic activity that transfers an acetyl group to a lysine residue of a substrate protein. Examples of the substrate include a histone protein and p53.

Bromo domain is a protein domain that recognizes an N-acetylated lysine residue. An N-acetylated lysine residue is found on, for example, an amino terminal tail of a histone protein.

“Cancer” refers to malignancies, and encompasses carcinoma, sarcoma, and hematologic malignancy. Specific examples of “cancer” include acoustic neuroma, acute leukemia, acute lymphocytic leukemia, acute myelocytic leukemia (monocytic, myeloblastic, adenocarcinoma, angiosarcoma, astrocytoma, myelomonocytic, and promyelocytic), acute T-cell leukemia, basal cell carcinoma, gall bladder/bile duct cancer, bladder cancer, brain cancer, breast cancer, bronchial cancer, cervical cancer, chondrosarcoma, choriocarcinoma, chorioepithelioma, urothelial carcinoma, chronic leukemia, chronic lymphocytic leukemia, chronic myelocytic (granulocytic) leukemia, chronic myleogeneous leukemia, colorectal cancer, cystadenocarcinoma, diffuse large B-cell lymphoma, dysproliferative changes (dysplasias and metaplasias), embryonal carcinoma, endometrial cancer, epithelial sarcoma, ependymoma, epithelial cancer, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinoma, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, stomach cancer, squamous cell carcinoma, synovioma, sweat gland carcinoma, thyroid cancer, Waldenstrom's macroglobulinemia, testicular tumor, uterine cancer, Wilms' tumor, malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, mesothelioma, etc. Specific examples of “tumor” include acute leukemia, chronic lymphocytic leukemia, chronic myelocytic leukemia, myelodysplastic syndrome, adult T cell leukemia/lymphoma, polycythemia vera, malignant lymphoma, myeloma, brain tumor, head and neck tumor, testicular tumor, Wilms' tumor, malignant melanoma, neuroblastoma, osteosarcoma, Ewing's sarcoma, chondrosarcoma, soft tissue sarcoma, skin cancer, etc.

“Cancer” is preferably SWI/SNF complex dysfunction cancer.

“SWI/SNF complex” is the general term for protein complexes comprised of a plurality of constituents regulating overall gene expression by ATP dependently changing the chromatin structure. Such complexes are roughly classified into three types of complexes (BAF complex, PBAF complex, and ncBAF complex) with different constituents (FIG. 13).

“SWI/SNF complex dysfunction cancer” is cancer with deficiency in the function of an SWI/SNF complex, and/or lack of or attenuation of expression of an SWI/SNF complex protein. Preferably, this is cancer with deficiency in the function of an SWI/SNF complex, and/or lack of expression of an SWI/SNF complex protein. More preferably, this is BAF complex dysfunction cancer.

“BAF complex dysfunction cancer” is cancer with deficiency in the function of a BAF complex and/or lack of or attenuation of expression of a BAF complex protein. Preferably, this is cancer with deficiency in the function of a BAF complex, and/or lack of or attenuation of expression of a BAF complex protein. More preferably, this is cancer with lack of or attenuation of expression of SMARCB1, INI1, SNF5, BAF47, SMARCA2, BAF190, BIS, BRM, NCBRS, SNF2, SNF2LA, SNF2L2, SMARCA4, BAF190A, BRG1, CSS4, MRD16, RTPS2, SNF2, SNF2B, SNF2L4, SNF2LB, ARID1A, B120, BAF250, BAF250a, BM029, C₁orf4, CSS2, ELD, MRD14, OSA1, P270, SMARCF1, hELD, hOSA1, ARID1B, 6A3-5, BAF250B, BRIGHT, CSS1, DAN15, ELD, OSA1, MRD12, OSA2, P250R, SMARCF2, SS18, SMARCL1, SSXT, or SYT. Most preferably, this is “SMARC deficient cancer”, “ARID deficient cancer”, or “SS18-SSX fusion cancer”.

“SMARC deficient cancer” is cancer with deficiency of an SMARC gene, and/or lack of or attenuation of expression of an SMARC protein. Preferably, this is cancer with deficiency of an SMARC gene, and/or lack of expression of an SMARC protein. More preferably, this is cancer with deficiency of an SMARCB1 gene, SMARCA2 gene, SMARCA4 gene, or SMARCA2/A4 gene. Specific examples thereof include malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, mesothelioma, pulmonary adenocarcinoma, large cell lung carcinoma, lung neuroendocrine tumor, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, thoracic cavity sarcoma, pleomorphic carcinoma, thoracic sarcoma, small cell carcinoma of the ovary, primary gallbladder tumor, and uterine sarcoma. Preferred examples thereof include malignant rhabdoid tumor and pulmonary adenocarcinoma.

“SMARCB1 deficient cancer” is cancer with deficiency of an SMARCB1 gene, and/or lack of or attenuation of expression of an SMARCB1 protein. Preferably, this is cancer with deficiency of an SMARCB1 gene, and/or loss of expression of an SMARCB1 protein. More preferably, this is cancer with deficiency of an SMARCB1 gene. Specific examples thereof include malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma. Preferably, this is malignant rhabdoid tumor.

“SMARCA2 deficient cancer” is cancer with deficiency of an SMARCA2 gene, and/or lack of or attenuation of expression of an SMARCA2 protein. Preferably, this is cancer with deficiency of an SMARCA2 gene, and/or lack of expression of an SMARCA2 protein. More preferably, this is cancer with deficiency of an SMARCA2 gene. Specific examples thereof include pulmonary adenocarcinoma, large cell lung carcinoma, lung neuroendocrine tumor, esophageal cancer, gastroesophageal junction cancer, and malignant rhabdoid tumor. Preferably, this is pulmonary adenocarcinoma.

“SMARCA4 deficient cancer” is cancer with deficiency of an SMARCA4 gene, and/or lack of or attenuation of expression of an SMARCA4 protein. Preferably, this is cancer with deficiency of an SMARCA4 gene, and/or lack of expression of an SMARCA4 protein. More preferably, this is cancer with deficiency of an SMARCA4 gene. Specific examples thereof include pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma. Preferably, this is pulmonary adenocarcinoma.

“SMARCA2 and SMARCA4 deficient cancer” is cancer with deficiency of an SMARCA2 gene and SMARCA4 gene, and/or lack of or attenuation of expression of an SMARCA2 protein and SMARCA4 protein. Preferably, this is cancer with deficiency of an SMARCA2 gene and SMARCA4 gene, and/or lack of expression of an SMARCA2 protein and SMARCA4 protein. More preferably, this is cancer with deficiency of an SMARCA2 gene and SMARCA4 gene. Specific examples thereof include pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer. Preferably, this is pulmonary adenocarcinoma.

“Deficiency of an SMARCB1 gene” is homozygous deficiency and/or heterozygous deficiency of an SMARCB1 gene, and preferably homozygous deficiency of an SMARCB1 gene.

“Deficiency of an SMARCA2 gene” is homozygous deficiency and/or heterozygous deficiency of an SMARCA2 gene, and preferably homozygous deficiency of an SMARCA2 gene.

“Deficiency of an SMARCA4 gene” is homozygous deficiency and/or heterozygous deficiency of an SMARCA4 gene, and preferably homozygous deficiency of an SMARCA4 gene.

“Deficiency of an SMARCA2 gene and SMARCA4 gene” is homozygous deficiency and/or heterozygous deficiency of an SMARCA2 gene and SMARCA4 gene, and preferably homozygous deficiency of an SMARCA2 gene and SMARCA4 gene.

“Lack of or attenuation of expression of an SMARCB1 protein”, “lack of or attenuation of expression of an SMARCA2 protein”, “lack of or attenuation of expression of an SMARCA4 protein”, and “lack of or attenuation of expression of an SMARCA2 protein and SMARCA4 protein” refer to either a case where expression is completely lost within tumor tissue, a case where lack of expression is observed in a mosaic pattern within tumor tissue, or a case where expression is attenuated in tumor tissue.

“SWI/SNF complex inhibitor” is a substance that suppresses the function of, reduces the function of, and/or reduces the expression of an SWI/SNF complex. “Reduced expression of an SWI/SNF complex” may manifest at any stage such as the level prior to transcription (e.g., genome stage), transcription level, post-transcription regulation level, translation level, or post-translation modification level.

“BAF complex inhibitor” is a substance that suppresses the function of, reduces the function of, and/or reduces the expression of a BAF complex. “Reduced expression of a BAF complex” may manifest at any stage such as the level prior to transcription (e.g., genome stage), transcription level, post-transcription regulation level, translation level, or post-translation modification level.

“SMARC inhibitor” is a substance that suppresses the function of, reduces the function of, and/or reduces the expression of SMARC. “Reduced expression of SMARC” may manifest at any stage such as the level prior to transcription (e.g., genome stage), transcription level, post-transcription regulation level, translation level, or post-translation modification level.

“SMARCB1 inhibitor” is a substance that suppresses the function of, reduces the function of, and/or reduces the expression of SMARCB1. “Reduced expression of SMARCB1” may manifest at any stage such as the level prior to transcription (e.g., genome stage), transcription level, post-transcription regulation level, translation level, or post-translation modification level.

“SMARCA2 inhibitor” is a substance that suppresses the function of, reduces the function of, and/or reduces the expression of SMARCA2. “Reduced expression of SMARCA2” may manifest at any stage such as the level prior to transcription (e.g., genome stage), transcription level, post-transcription regulation level, translation level, or post-translation modification level.

“SMARCA4 inhibitor” is a substance that suppresses the function of, reduces the function of, and/or reduces the expression of SMARCA4. “Reduced expression of SMARCA4” may manifest at any stage such as the level prior to transcription (e.g., genome stage), transcription level, post-transcription regulation level, translation level, or post-translation modification level.

“SMARCA2/A4 inhibitor” is a substance that suppresses the function of, reduces the function of, and/or reduces the expression of SMARCA2 and SMARCA4, including combined agents and concomitant use of an SMARCA2 inhibitor and an SMARCA4 inhibitor. “Reduced expression of SMARCA2 and SMARCA4” may manifest at any stage such as the level prior to transcription (e.g., genome stage), transcription level, post-transcription regulation level, translation level, or post-translation modification level.

“ARID deficient cancer” is cancer with deficiency of an ARID gene and/or lack of or attenuation of expression of an ARID protein. Preferably, this is cancer with deficiency of an ARID gene and/or lack of expression of an ARID protein. More preferably, this is cancer with deficiency of an ARID1A gene, ARID1B gene, or ARID1A/1B gene. Specific examples thereof include ovarian cancer, gastric cancer, bile duct cancer, pancreatic cancer, uterine cancer, neuroblastoma, colon cancer, bladder cancer, liver cancer, melanoma, breast cancer, medulloblastoma, and neuroblastoma. Preferably, this is ovarian cancer.

“ARID1A deficient cancer” is cancer with deficiency of an ARID1A gene and/or lack of or attenuation of expression of an ARID1A protein. Preferably, this is cancer with deficiency of an ARID1A gene and/or lack of expression of an ARID1A protein. More preferably, this is cancer with deficiency of an ARID1A gene. Specific examples thereof include ovarian cancer, gastric cancer, bile duct cancer, pancreatic cancer, uterine cancer, neuroblastoma, colon cancer, and bladder cancer. Preferably, this is ovarian cancer.

“ARID1B deficient cancer” is cancer with deficiency of an ARID1B gene and/or lack of or attenuation of expression of an ARID1B protein. Preferably, this is cancer with deficiency of an ARID1B gene and/or lack of expression of an ARID1B protein. More preferably, this is cancer with deficiency of an ARID1B gene. Specific examples thereof include ovarian cancer, colon cancer, pancreatic cancer, liver cancer, melanoma, breast cancer, medulloblastoma, uterine cancer, bladder cancer, and gastric cancer. Preferably, this is ovarian cancer.

“ARID1A and ARID1B deficient cancer” is cancer with deficiency of an ARID1A gene and ARID1B gene and/or lack of or attenuation of expression of an ARID1A protein and ARID1B protein. Preferably, this is cancer with deficiency of an ARID1A gene and ARID1B gene and/or lack of expression of an ARID1A protein and ARID1B protein. More preferably, this is cancer with deficiency of an ARID1A gene and ARID1B gene. Specific examples thereof include ovarian cancer, colon cancer, uterine cancer, neuroblastoma, bladder cancer, and gastric cancer. Preferably, this is ovarian cancer.

“Deficiency of an ARID1A gene” is homozygous deficiency and/or heterozygous deficiency of an ARID1A gene, and preferably homozygous deficiency of an ARID1A gene.

“Deficiency of an ARID1B gene” is homozygous deficiency and/or heterozygous deficiency of an ARID1B gene, and preferably homozygous deficiency of an ARID1B gene.

“Deficiency of an ARID1A gene and ARID1B gene” is homozygous deficiency and/or heterozygous deficiency of an ARID1A gene and ARID1B gene, and preferably homozygous deficiency of an ARID1A gene and ARID1B gene.

“Lack of or attenuation of expression of an ARID1A protein”, “lack of or attenuation of expression of an ARID1B protein”, and “lack of or attenuation of expression of an ARID1A protein and ARID1B protein” refer to either a case where expression is completely lost within tumor tissue, a case where lack of expression is observed in a mosaic pattern within tumor tissue, or a case where expression is attenuated in tumor tissue.

“ARID inhibitor” is a substance that suppresses the function of, reduces the function of, and/or reduces the expression of ARID. “Reduced expression of ARID” may manifest at any stage such as the level prior to transcription (e.g., genome stage), transcription level, post-transcription regulation level, translation level, or post-translation modification level.

“ARID1A inhibitor” is a substance that suppresses the function of, reduces the function of, and/or reduces the expression of ARID1A. “Reduced expression of ARID1A” may manifest at any stage such as the level prior to transcription (e.g., genome stage), transcription level, post-transcription regulation level, translation level, or post-translation modification level.

“ARID1B inhibitor” is a substance that suppresses the function of, reduces the function of, and/or reduces the expression of ARID1B. “Reduced expression of ARID1B” may manifest at any stage such as the level prior to transcription (e.g., genome stage), transcription level, post-transcription regulation level, translation level, or post-translation modification level.

“ARID1A/1B inhibitor” is a substance that suppresses the function of, reduces the function of, and/or reduces the expression of ARID1A and ARID1B, including combined agents and concomitant use of an ARID1A inhibitor and an ARID1B inhibitor. “Reduced expression of ARID1A and ARID1B” may manifest at any stage such as the level prior to transcription (e.g., genome stage), transcription level, post-transcription regulation level, translation level, or post-translation modification level.

“SS18-SSX fusion cancer” is cancer wherein a SS18 gene is fused to an SSX gene. Specific examples thereof include synovial sarcoma and Ewing's sarcoma. Preferably, this is synovial sarcoma.

“Fusion of an SS18 gene to an SSX gene” refers to fusion of an SS18 gene on the 18^{th} chromosome to an SSX1, SSX2, or SSX4 gene on the X chromosome.

“CBP/P300 deficient cancer” is cancer with deficiency of a CBP and/or P300 gene and/or lack of or attenuation of expression of a CBP and/or P300 protein. Preferably, this is cancer with deficiency of a CBP and/or P300 gene and/or lack of expression of a CBP and/or P300 protein. More preferably, this is cancer with deficiency of a CBP and/or P300 gene. Specific examples thereof include lung cancer, bladder cancer, lymphoma, and adenoid cystic carcinoma.

“Deficiency of a CBP/P300 gene” is homozygous deficiency and/or heterozygous deficiency of a CBP and/or P300 gene, and preferably homozygous deficiency of a CBP and/or P300 gene.

“Lack of or attenuation of expression of a CBP/P300 protein” refers to either a case where expression is completely lost within tumor tissue, a case where lack of expression is observed in a mosaic pattern within tumor tissue, or a case where expression is attenuated in tumor tissue.

“Low molecular weight compound” refers to an “organic low molecular weight compound” or “inorganic low molecular weight compound” with a molecular weight of less than 10000. Preferred examples of “low molecular weight compound” include “organic low molecular weight compound”.

The molecular weight of a “low molecular weight compound” is preferably 5000 or less, more preferably 3000 or less, still more preferably 2000 or less, and most preferably 1000 or less.

“Nucleic acid” refers to a molecule with nucleotides consisting of a base, saccharide, and phosphoric acid connected via a phosphodiester bond. Nucleic acids include ribonucleic acids (RNA) and deoxyribonucleic acids (DNA), including artificially modified or substituted nucleic acids and nucleic acid precursors converted into a nucleic acid in vivo. Examples of artificially modified or substituted nucleic acids include 5-substituted pyrimidine, 6-azapyrimidine, and N-2, N-6, and O-6 substituted purine (including 2-aminopropyladenine), 5-propynyluracil, 5-propynylcytosine, etc. As an artificially modified or substituted nucleic acid, a modified nucleic acid (bridged nucleic acid (BNA)) having position 2′ and position 4′ of the nucleic acid linked (bridged) and having two cyclic structures (bicyclic), etc. can be used. Modified nucleic acids such as a peptide nucleic acid, locked nucleic acid, morpholino nucleic acid, and thio nucleic acid can also be used. Examples of “nucleic acid” include “antisense nucleic acid”, “ribozyme”, and “nucleic acid having RNAi activity”. Preferred examples thereof include antisense nucleic acid, ribozyme, nucleic acid having RNAi activity for a transcriptional product of a gene encoding CBP or P300, and precursors thereof.

Examples of “antisense nucleic acid” include a polydeoxyribonucleotide comprising 2-deoxy-D-ribose, polyribonucleotide comprising D-ribose, other types of polynucleotides which are N-glycoside of a pyrimidine base or purine, other polymers having a non-nucleotide backbone (e.g., commercially available protein nucleic acids and synthetic sequence specific nucleic acid polymers), other polymers comprising a special bond (provided that the polymer comprises a nucleotide with a configuration that allows pairing of bases or attachment of a base as seen in a DNA or RNA), etc. Antisense nucleic acids may be a double stranded DNA, single stranded DNA, double stranded RNA, single stranded RNA, DNA:RNA hybrid, unmodified polynucleotide (or unmodified oligonucleotide), those added with a known modification such as those with a label known in the art, those that are capped, those that are methylated, those with one or more naturally-occurring nucleotides substituted with an analog, those with intramolecular nucleotide modification, such as those with a non-charged bond (e.g., methylphosphonate, phosphotriester, phosphoramidate, carbamate, etc.), those with a charged bond or sulfur containing bond (e.g., phosphorothioate, phosphorodithioate, etc.), such as those having a side chain group of a protein (e.g., nuclease, nuclease inhibitor, toxin, antibody, signal peptide, poly-L-lysine, etc.) or a saccharide (e.g., monosaccharide, etc.), those having an intercalating compound (e.g., acridine, psoralen, etc.), those containing a chelate compound (e.g., metals, radioactive metals, boron, oxidizing metals, etc.), those containing an alkylating agent, or those having a modified bond (for example, α-anomer nucleic acid, etc.). In this regard, “nucleoside”, “nucleotide”, and “nucleic acid” may include not only those containing purine and pyrimidine bases, but also those containing another modified heterocyclic base. Such modified products may contain a methylated purine and pyrimidine, acylated purine and pyrimidine, or other heterocycles. Modified nucleosides and modified nucleotides may also have a modified saccharide moiety, e.g., one or more hydroxyl groups substituted with halogen, aliphatic group, etc., or converted to a functional group such as ether or amine.

As described above, an antisense nucleic acid may be a DNA or RNA, or a DNA:RNA chimera. If an antisense nucleic acid is a DNA, an RNA:DNA hybrid formed by a target RNA and antisense DNA can be recognized by endogenous RNase H and induce selective degradation of the target RNA. Thus, in case of an antisense DNA directing degradation by RNase H, a target sequence may be not only a sequence in an mRNA, but also a sequence of an intron region in an early translation product of a CBP gene or P300 gene. An intron sequence can be determined by comparing the genomic sequence with a cDNA base sequence by using a homology search program such as BLAST or FASTA.

“Ribozyme”, in a narrow sense, refers to an RNA having enzymatic activity for cleaving a nucleic acid, but is used as a concept that also encompasses DNAs herein, as long as it has sequence specific nucleic acid cleaving activity. Ribozymes with highest versatility are self-splicing RNAs seen in infectious RNAs such as viroids and virusoids. Hammerhead ribozymes, hairpin ribozymes, etc. are known. Hammerhead ribozymes exert enzymatic activity with about 40 bases, and can specifically cleave only the target mRNA by having sequences complementary to the desired site of cleavage of mRNA at several bases each (total of about 10 bases) at both ends adjacent to a portion having a hammerhead structure. This type of ribozyme uses only RNA as a substrate and thus has an additional advantage of not attacking genomic DNAs. When an mRNA of a CBP gene or P300 gene itself has a double stranded structure, a target sequence can be a single strand by using a hybrid ribozyme linked to an RNA motif derived from a viral nucleic acid that can specifically bind to an RNA helicase (Proc. Natl. Acad. Sci. USA. 2001; 98(10): 5572-5577). Furthermore, when a ribozyme is used in a form of an expression vector comprising a DNA encoding the same, a hybrid ribozyme further linked to a sequence with an altered tRNA can be prepared to promote migration of a transcriptional product into the cytoplasm (Nucleic Acids Res. 2001; 29(13): 2780-2788).

“Nucleic acid having RNAi activity” refers to a nucleic acid that induces a phenomenon of degrading an mRNA of a target gene when introduced into a cell, known as RNA interference (RNAi). Representative examples include siRNA and shRNA. An siRNA is a double stranded RNA consisting of an oligo RNA complementary to an mRNA of a target gene and a complementary strand thereof. An siRNA can be designed in accordance with rules proposed by, for example, Elbashir et al. (Genes Dev., 2001; 15(2): 188-200) or Teramoto et al. (FEBS Lett. 2005; 579(13): 2878-2882), based on cDNA sequence information of a target gene. The target sequence of an siRNA has a length of, in principle, 15 to 50 bases, and preferably 19 to 27 bases. An siRNA may have an additional base on the 5′ or 3′ terminus. The length of the additional bases is generally about 2 to 4 bases, and the full length of an siRNA is 19 bases or longer. The additional bases may be a DNA or RNA, but the stability of a nucleic acid may be improved when a DNA is used. Examples of the sequence of such additional bases include, but are not limited to, ug-3′, uu-3′, tg-3′, tt-3′, ggg-3′, guuu-3′, gttt-3′, ttttt-3′, uuuuu-3′, etc. An siRNA also may have an overhang on the 3′ terminus. Specific examples thereof include those with an addition of dTdT (dT represents a deoxythimidine residue of a deoxyribonucleic acid). This may also be a blunt end with no addition to a terminus. An siRNA may have different number of bases on the sense strand and antisense strand. Examples thereof include an aiRNA with an antisense strand having an overhand at the 3′ terminus and the 5′ terminus. A typical aiRNA has an antisense strand consisting of 21 bases and a sense strand consisting of 15 bases, with an overhang structure of 3 bases on both ends of the antisense strand (Nat. Biotechnol. 2008; 26(12): 1379-1382, International Publication No. WO 2009/029688). Further, a short hairpin RNA (shRNA) which is a precursor of an siRNA can be designed by selecting any appropriate linker sequence (e.g., about 5 to 25 bases) that can form a loop structure and linking the sense strand and the antisense strand described above via said linker sequence.

A ribonucleoside molecule constituting an siRNA may also be modified in the same manner as the antisense nucleic acid described above in order to improve the stability, specific activity, etc. However for an siRNA, RNAi activity may be lost if all ribonucleoside molecules in a naturally-occurring RNA are replaced with a modified form. Thus, it is necessary to introduce the minimum number of modified nucleosides with which an RISC complex can function. As a specific example of said modification, some of nucleotide molecules constituting an siRNA can be replaced with a naturally-occurring DNA or an RNA subjected to various chemical modifications in order to improve stability (chemical and/or to an enzyme) of specific activity (affinity with RNA) (see Trends Biochem Sci. 1992; 17(9): 334-339). For example, to prevent degradation due to a hydrolase such as nuclease, a phosphoric acid residue (phosphate) of each nucleotide constituting an siRNA can be replaced with, for example, a chemically modified phosphoric acid residue such as phosphorothioate (PS), methylphosphonate, or phosphorodithionate. A hydroxyl group at position 2′ of a saccharide (ribose) of each nucleotide may be substituted with —OR(R═CH₃ (2′-O-M^{e}), CH₂CH₂OCH₃ (2′-O-MOE), CH₂CH₂NHC(NH)NH₂, CH₂CONHCH₃, CH₂CH₂CN, etc.), or a fluorine atom (—F). Furthermore, a chemical modification may be applied to a base moiety (pyrimidine, purine). Examples thereof include introduction of a methyl group or cationic functional group to position 5 of a pyrimidine base, substitution of a carbonyl group at position 2 to a thiocarbonyl, etc. In addition, the modification method for the antisense nucleic acid described above can be used. Alternatively, a chemical modification that replaces a portion of an RNA in an siRNA with a DNA (2′-deoxy conversion, 2′-H) may be applied. Further, an artificial nucleic acid wherein position 2′ and position 4′ of a saccharide (ribose) are bridged with —O—CH₂— to immobilize the conformation to an N-form (LNA, Locked nucleic acid) may be used. Further, a sense strand and antisense strand constituting an siRNA may be chemically attached to, via a linker, a ligand specifically recognizing a receptor that is present on the cell surface layer, peptide, sugar chain, antibody, lipid, positive charge, oligoarginine which molecular structurally adsorbs to and penetrates a cell membrane surface layer, Tat peptide, Rev peptide, Ant peptide, etc.

With regard to information on gene sequence that can be targeted by an antisense nucleic acid, ribozyme, or siRNA, the position of a human CREBBP gene encoding human CBP in the genome is known as GenBank Accession Number: NC_000016.10 (3725054 to 3880727, complementary strand, Assembly: GRCh38.p13). A representative mRNA sequence of human CREBBP is set forth in GenBank Accession Number: NM_001079846.1 (SEQ ID NO: 4) or NM_004380.3 (SEQ ID NO: 5). The position of a human EP300 gene encoding human P300 in the genome is known as GenBank Accession Number: NC_000022.11 (41092592 to 41180077, Assembly: GRCh38.p13). A representative mRNA sequence of human EP300 is set forth in GenBank Accession Number: NM 001362843.2 (SEQ ID NO: 6) or NM_001429.4 (SEQ ID NO: 7).

With regard to information on gene sequence that can be targeted by an antisense nucleic acid, ribozyme, or siRNA, the position of a human SMARCB1 gene encoding human SMARCB1 in the genome is known as GenBank Accession Number: NC_000022.11 (23786966 to 23838009, Assembly: GRCh38.p13). A representative mRNA sequence of human SMARCB1 is set forth in GenBank Accession Number: NM_003073.5 (SEQ ID NO: 8) or NM 001007468.3 (SEQ ID NO: 9).

With regard to information on gene sequence that can be targeted by an antisense nucleic acid, ribozyme, or siRNA, the position of a human SMARCA2 gene encoding human SMARCA2 in the genome is known as GenBank Accession Number: NC_000009.12 (2015347 to 2193624, Assembly: GRCh38.p13). A representative mRNA sequence of human SMARCA2 is set forth in GenBank Accession Number: NM_003070.5 (SEQ ID NO: 10) or NM_139045.4 (SEQ ID NO: 11).

With regard to information on gene sequence that can be targeted by an antisense nucleic acid, ribozyme, or siRNA, the position of a human SMARCA4 gene encoding human SMARCA4 in the genome is known as GenBank Accession Number: NC_000019.10 (10960999 to 11062277, Assembly: GRCh38.p13). A representative mRNA sequence of human SMARCA4 is set forth in GenBank Accession Number: NM 001387283.1 (SEQ ID NO: 12) or NM_001128844.3 (SEQ ID NO: 13).

With regard to information on gene sequence that can be targeted by an antisense nucleic acid, ribozyme, or siRNA, the position of a human ARID1A gene encoding human ARID1A in the genome is known as GenBank Accession Number: NC_000001.11 (26696015 to 26782104, Assembly: GRCh38.p13). A representative mRNA sequence of human ARID1A is set forth in GenBank Accession Number: NM_006015.6 (SEQ ID NO: 14) or NM_139135.4 (SEQ ID NO: 15).

With regard to information on gene sequence that can be targeted by an antisense nucleic acid, ribozyme, or siRNA, the position of a human ARID1B gene encoding human ARID1B in the genome is known as GenBank Accession Number: NC_000006.12 (156776026 to 157210779, Assembly: GRCh38.p13). A representative mRNA sequence of human ARID1B is set forth in GenBank Accession Number: NM 001363725.2 (SEQ ID NO: 16), NM_001371656.1 (SEQ ID NO: 17), NM_001374820.1 (SEQ ID NO: 18), NM_001374828.1 (SEQ ID NO: 19), or NM_017519.3 (SEQ ID NO: 20).

The term “SMARCB1” as used herein refers to any naturally-occurring SMARCB1 derived from any vertebrate source including mammals such as primates (e.g., human) and rodents (e.g., mouse and rat), unless specifically noted otherwise. The term encompasses unprocessed SMARCB1 and any form of SMARCB1 resulting from processing in a cell. The term also encompasses naturally-occurring variants of SMARCB1 such as splice variants and allelic variants. Human SMARCB1 is registered as UniProt Accession Number: Q12824. Representative amino acid sequences of human SMARCB1 are set forth in UniProt Q12824-1 (SEQ ID NO: 21) and UniProt Q12824-2 (SEQ ID NO: 22).

The term “SMARCA2” as used herein refers to any naturally-occurring SMARCA2 derived from any vertebrate source including mammals such as primates (e.g., human) and rodents (e.g., mouse and rat), unless specifically noted otherwise. The term encompasses unprocessed SMARCA2 and any form of SMARCA2 resulting from processing in a cell. The term also encompasses naturally-occurring variants of SMARCA2 such as splice variants and allelic variants. Human SMARCA2 is registered as UniProt Accession Number: P51531. Representative amino acid sequences of human SMARCA2 are set forth in UniProt P51531-1 (SEQ ID NO: 23) and UniProt P51531-2 (SEQ ID NO: 24).

The term “SMARCA4” as used herein refers to any naturally-occurring SMARCA4 derived from any vertebrate source including mammals such as primates (e.g., human) and rodents (e.g., mouse and rat), unless specifically noted otherwise. The term encompasses unprocessed SMARCA4 and any form of SMARCA4 resulting from processing in a cell. The term also encompasses naturally-occurring variants of SMARCA4 such as splice variants and allelic variants. Human SMARCA4 is registered as UniProt Accession Number: P51532. Representative amino acid sequences of human SMARCA4 are set forth in UniProt P51532-1 (SEQ ID NO: 25) and UniProt P51532-2 (SEQ ID NO: 26).

The term “ARID1A” as used herein refers to any naturally-occurring ARID1A derived from any vertebrate source including mammals such as primates (e.g., human) and rodents (e.g., mouse and rat), unless specifically noted otherwise. The term encompasses unprocessed ARID1A and any form of ARID1A resulting from processing in a cell. The term also encompasses naturally-occurring variants of ARID1A such as splice variants and allelic variants. Human ARID1A is registered as UniProt Accession Number: 014497. Representative amino acid sequences of human ARID1A are set forth in UniProt 014497-1 (SEQ ID NO: 27), 014497-2 (SEQ ID NO: 28), and UniProt 014497-3 (SEQ ID NO: 29).

The term “ARID1B” as used herein refers to any naturally-occurring ARID1B derived from any vertebrate source including mammals such as primates (e.g., human) and rodents (e.g., mouse and rat), unless specifically noted otherwise. The term encompasses unprocessed ARID1B and any form of ARID1B resulting from processing in a cell. The term also encompasses naturally-occurring variants of ARID1B such as splice variants and allelic variants. Human ARID1B is registered as UniProt Accession Number: Q8NFD5. Representative amino acid sequences of human ARID1B are set forth in UniProt Q8NFD5-1 (SEQ ID NO: 30), Q8NFD5-2 (SEQ ID NO: 31), Q8NFD5-3 (SEQ ID NO: 32), and UniProt Q8NFD5-4 (SEQ ID NO: 33).

The term “SS18” as used herein refers to any naturally-occurring SS18 derived from any vertebrate source including mammals such as primates (e.g., human) and rodents (e.g., mouse and rat), unless specifically noted otherwise. The term encompasses unprocessed SS18 and any form of SS18 resulting from processing in a cell. The term also encompasses naturally-occurring variants of SS18 such as splice variants and allelic variants. Human SS18 is registered as UniProt Accession Number: Q15532. Representative amino acid sequences of human SS18 are set forth in UniProt Q15532-1 (SEQ ID NO: 34) and UniProt Q15532-2 (SEQ ID NO: 35).

As used herein, “prevention” is an act of administering an active ingredient in the present disclosure to an individual who has not been diagnosed as having developed the target disease, and is intended to for example prevent development of the disease.

As used herein, “therapy” is an act of administering an active ingredient of the present disclosure to an individual (patient) diagnosed as having developed a disease by a physician, intended to, for example, alleviate the disease or symptom, not increase carcinoma, or revert back to the state before the development of the disease.

Even if the objective of administration is prevention of exacerbation of the disease or symptom or prevention of increase in the carcinoma, the administration is a therapeutic act if administered to a patient.

When administering the CBP/P300 inhibitor of the present disclosure, the amount used varies depending on the symptom, age, administration method, etc., but an effect is expected by administering, for intravenous injection, 0.01 mg (preferably 0.1 mg) as the lower limit to 1000 mg (preferably 100 mg) as the upper limit per day for an adult, separated into one or several doses, depending on the symptom. Examples of the dosing schedule thereof include a single dose, once daily administration for 3 consecutive days, twice daily administration for 7 consecutive days, etc. Each of the administration methods described above can also be repeated with an interval of about 1 day to about 60 days.

The CBP/P300 inhibitor of the present disclosure can be administered directly or after being formulated into a suitable dosage form through parenteral or oral administration. Examples of the dosage form include, but are not limited to, a tablet, a capsule, powder, a granule, a liquid agent, a suspension, an injection, a patch, a poultice, etc. A formulation can be manufactured by a known method using a pharmaceutically acceptable additive.

An excipient, disintegrant, binding agent, fluidizer, lubricant, coating agent, solubilizing agent, solubilizing adjuvant, thickener, dispersant, stabilizing agent, sweetener, flavoring agent, etc. can be used as an additive in accordance with the objective. Specific examples of the additive include lactose, mannitol, crystalline cellulose, low substituted hydroxypropyl cellulose, corn starch, partially pregelatinized starch, carmellose calcium, croscarmellose sodium, hydroxypropyl cellulose, hydroxypropylmethyl cellulose, polyvinyl alcohol, magnesium stearate, sodium stearyl fumarate, polyethylene glycol, propylene glycol, titanium oxide, talc, etc.

The CBP/P300 inhibitor of the present disclosure can be parenterally or orally administered, but is preferably administered by an oral method.

The CBP/P300 inhibitor of the present disclosure can be used concomitantly with another drug in order to enhance the effect thereof. Specifically, the CBP/P300 inhibitor of the present disclosure can be used concomitantly with a drug such as a hormonal therapy agent, a chemotherapeutic agent, an immunotherapeutic agent, or an agent inhibiting a cell growth factor and its receptor action. Hereinafter, a drug that can be concomitantly used with the CBP/P300 inhibitor of the present disclosure is abbreviated as the concomitantly used drug.

Although the CBP/P300 inhibitor of the present disclosure exhibits excellent anticancer action when used as a single agent, the effect thereof can be further enhanced, or the QOL of a patient can be improved, by concomitantly using one or several of the concomitantly used drugs described above (concomitant use of multiple drugs).

Examples of “hormonal therapy agent” include fosfestrol, diethylstilbestrol, chlorotrianisene, medroxyprogesterone acetate, megestrol acetate, chlormadinone acetate, cyproterone acetate, danazol, dienogest, asoprisnil, allylestrenol, gestrinone, nomegestol, tadenan, mepartricin, raloxifene, ormeroxifene, levormeloxifene, antiestrogens (e.g., tamoxifen citrate, toremifene citrate, etc.), pill formulations, mepitiostane, testolactone, aminoglutethimide, LH-RH derivatives (LH-RH agonists (e.g., goserelin acetate, buserelin, leuprorelin, etc.) and LH-RH antagonists), droloxifene, epitiostanol, ethinylestradiol sulfonate, aromatase inhibitors (e.g., fadrozole hydrochloride, anastrozole, letrozole, exemestane, vorozole, formestane, etc.), flutamide, bicalutamide, nilutamide, androgen receptor antagonists (e.g., apalutamide and enzalutamide), androgen synthesis inhibitors (e.g., abiraterone, etc.), adrenocortical hormone agents (e.g., dexamethasone, prednisolone, betamethasone, triamcinolone, etc.), retinoids, drugs that slow the metabolism of retinoids (e.g., liarozole, etc.), etc.

For example, an alkylating agent, antimetabolite, anticancer antibiotic, plant derived anticancer agent, molecularly targeted therapy agent, immunomodulator, other chemotherapeutic agent, etc. are used as a “chemotherapeutic agent”. Representative examples thereof are described below.

Examples of “alkylating agents” include nitrogen mustard, nitrogen mustard N-oxide hydrochloride, chlorambucil, cyclophosphamide, ifosfamide, thiotepa, carboquone, improsulfan tosylate, busulfan, nimustine hydrochloride, mitobronitol, melphalan, dacarbazine, ranimustine, estramustine phosphate sodium, triethylenemelamine, carmustine, lomustine, streptozocin, pipobroman, etoglucide, carboplatin, cisplatin, miboplatin, nedaplatin, oxaliplatin, altretamine, ambamustine, dibrospidium chloride, fotemustine, prednimustine, pumitepa, ribomustin, temozolomide, thiotepa, treosulfan, trofosfamide, zinostatin stimalamer, adozelesin, cystemustine, bizelesin, DDS formulations thereof, etc.

Examples of “antimetabolite” include mercaptopurine, 6-mercaptopurine riboside, thioinosine, methotrexate, pemetrexed, enocitabine, cytarabine, cytarabine ocfosfate, ancitabine hydrochloride, 5-FU based agents (e.g., fluorouracil, tegafur, UFT, doxifluridine, carmofur, galocitabine, emitefur, capecitabine, etc.), aminopterin, nelzarabine, leucovorin calcium, tabloid, butocin, calcium folinate, calcium levofolinate, cladribine, emitefur, fludarabine, gemcitabine, hydroxycarbamide, pentostatin, piritrexim, idoxuridine, mitoguazone, tiazofurin, ambamustine, bendamustine, DDS formulations thereof, etc.

Examples of “anticancer antibiotic” include actinomycin D, actinomycin C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarkomycin, carzinophilin, mitotane, zorubicin hydrochloride, mitoxantrone hydrochloride, idarubicin hydrochloride, DDS formulations thereof, etc.

Examples of “plant derived anticancer agent” include etoposide, etoposide phosphate, vinblastine sulfate, vincristine sulfate, vindesine sulfate, teniposide, paclitaxel, docetaxel, DJ-927, vinorelbine, irinotecan, topotecan, DDS formulations thereof, etc.

Examples of “molecularly targeted therapy agent” include imatinib, gefitinib, erlotinib, sorafenib, dasatinib, sunitinib, nilotinib, lapatinib, pazopanib, ruxolitinib, crizotinib, vemurafenib, vandetanib, ponatinib, cabozantinib, tofacitinib, regorafenib, bosutinib, axitinib, dabrafenib, trametinib, nintedanib, idelalisib, ceritinib, lenvatinib, palbociclib, alectinib, afatinib, osimertinib, ribociclib, abemaciclib, brigatinib, neratinib, copanlisib, cobimetinib, ibrutinib, acalabrutinib, encorafenib, binimetinib, baricitinib, fostamatinib, lorlatinib, erdafitinib, entrectinib, dacomitinib, sirolimus, everolimus, temsirolimus, olaparib, rucaparib, niraparib, venetoclax, azacitidine, decitabine, vorinostat, panobinostat, romidepsin, bortezomib, carfilzomib, tazemetostat, ixazomib, etc.

Examples of “immunomodulator” include lenalidomide, pomalidomide, etc.

Examples of “other chemotherapeutic agent” include sobuzoxane, etc.

Examples of “immunotherapeutic agent (BRM)” include picibanil, krestin, sizofiran, lentinan, ubenimex, interferon, interleukin, macrophage colony stimulating factor, granulocyte-colony stimulating factor, erythropoietin, lymphotoxin, BCG vaccine, Corynebacterium parvum, levamisole, polysaccharide K, procodazole, anti-CTLA4 antibody, anti-PD-1 antibody, anti-PD-L1 antibody, and Toll-like Receptor agonists (e.g., TLR7 agonist, TLR8 agonist, TLR9 agonist, etc.).

The cell growth factor in an agent inhibiting a cell growth factor and its receptor action can be any substance, as long as it is a substance promoting cell growth. A cell growth factor is generally a peptide having a molecular weight of 20,000 or less and exerting action at a low concentration by binding with a receptor. Specific examples thereof include EGF (epidermal growth factor) or substances having substantially the same activity as EGF (e.g., TGF-alpha, etc.), insulin or substances having substantially the same activity as insulin (e.g., insulin, IGF (insulin-like growth factor)-1, IGF-2, etc.), FGF (fibroblast growth factor) or substances having substantially the same activity as FGF (e.g., acidic FGF, basic FGF, KGK (keratinocyte growth factor), FGF-10, etc.), and other cell growth factors (e.g., CSF (colony stimulating factor), EPO (erythropoietin), IL-2 (interleukin-2), NGF (nerve growth factor), PDGF (platelet-derived growth factor), TGF-beta (transforming growth factor beta), HGF (hepatocyte growth factor), VEGF (vascular endothelial growth factor), heregulin, angiopoietin, etc.).

The dosing period of the substance of the present disclosure and a concomitantly used drug is not limited. They can be administered simultaneously or differentially to a target of administration. The substance of the present disclosure and a concomitantly used drug can also be prepared as a combined drug. The amount of concomitantly used drug to be administered can be appropriately selected based on clinically used doses. The blend ratio of the substance of the present disclosure and a concomitantly used drug can be appropriately selected depending on the subject of administration, route of administration, target disease, symptom, combination, etc. If, for example, the subject of administration is a human, 0.01 to 100 parts by weight of concomitantly used drug can be used with respect to 1 part by weight of the compound of the present disclosure. They can also be used in combination with an agent (concomitantly used drug) such as an antiemetic, sleep inducing agent, or anticonvulsive in order to suppress side effects thereof.

Examples of “pharmaceutically acceptable salt” include acid addition salts and base addition salts. Examples of acid addition salts include inorganic acid salts such as hydrochloric acid salt, hydrobromic acid salt, sulfuric acid salt, hydroiodic acid salt, nitric acid salt, and phosphoric acid salt, and organic acid salts such as citric acid salt, oxalic acid salt, phthalic acid salt, fumaric acid salt, maleic acid salt, succinic acid salt, malic acid salt, acetic acid salt, formic acid salt, propionic acid salt, benzoic acid salt, trifluoroacetic acid salt, methanesulfonic acid salt, benzenesulfonic acid salt, p-toluenesulfonic acid salt, and camphorsulfonic acid salt. Examples of base addition salts include inorganic base salts such as sodium salt, potassium salt, calcium salt, magnesium salt, barium salt, and aluminum salt, organic base salts such as trimethylamine, triethylamine, pyridine, picoline, 2,6-lutidine, ethanolamine, diethanolamine, triethanolamine, tromethamine [tris(hydroxymethyl)methylamine], tert-butylamine, cyclohexylamine, dicyclohexylamine, and N—N-dibenzylethylamine, etc. Furthermore, examples thereof include amino acid salts of a basic or acidic amino acid such as arginine, lysine, ornithine, aspartic acid, and glutamic acid.

Deuterated compounds prepared by converting any one or two or more of 1H of compounds represented by formulas (1) to (23) to 2H(D) are also encompassed by the compounds represented by formulas (1) to (23) in the present disclosure.

The present disclosure encompasses the compounds represented by formulas (1) to (23) and pharmaceutically acceptable salts thereof. The compound of the present disclosure can also be in a form of a hydrate and/or solvate of various solvents (ethanolate, etc.) Thus, such hydrates and/or solvates are also encompassed by the compound of the present disclosure. The present disclosure also encompasses any tautomer, any existing stereoisomers, crystalline forms in any form of formulas (1) to (23) of the present disclosure, and mixtures thereof.

Formulas (1) to (23) may have enantiomers based on an optically-active center, atropisomers based on axial or planar chirality resulting from restriction of intramolecular rotation, other stereoisomers, tautomers, geometric isomers, etc., which are encompassed by formulas (1) to (23) including all possible isomers and mixtures thereof.

As used herein, “C₁-6” means that the number of carbon atoms is 1 to 6. The same applies to other numbers. For example, “C₁₋₄” means that the number of carbon atoms is 1 to 4.

As used herein, “heteroatom” refers to an oxygen atom, a nitrogen atom, a sulfur atom, a phosphorous atom, a silicon atom, etc. (including oxidized forms of nitrogen, sulfur, phosphorous, or silicon and any quaternized form of nitrogen).

As used herein, “halogen atom” refers to a fluorine atom, chlorine atom, bromine atom, or iodine atom. A “halogen atom” is also referred to as “halogen”. A halogen atom may also be referred to “halo” or “halogeno” when substituted with another group.

As used herein, “alkyl” or “alkyl group” refers to a linear or branched saturated hydrocarbon group. For example, “C₁₋₆ alkyl” or “C₁₋₆ alkyl group” refers to a linear or branched saturated hydrocarbon group with 1 to 6 carbon atoms. Examples of a C₁₋₆ alkyl group include a “C₁₋₄ alkyl group”, and a “C₁₋₃ alkyl group”. Specific examples of “C₁₋₃ alkyl group” include methyl, ethyl, propyl, 1-methylethyl, etc. Specific examples of “C₁₋₄ alkyl group” include, in addition to the specific examples for the “C₁₋₃ alkyl group” described above, butyl, 1,1-dimethylethyl, 1-methylpropyl, 2-methylpropyl, etc. Specific examples of “C₁₋₆ alkyl group” include, in addition to the specific examples for the “C₁₋₄ alkyl group” described above, pentyl, 1,1-dimethylpropyl, 1,2-dimethylpropyl, 1-methylbutyl, 2-methylbutyl, 4-methylpentyl, 3-methylpentyl, 2-methylpentyl, 1-methylpentyl, hexyl, etc.

“Alkyl” or “alkyl group” is optionally substituted. “Optionally substituted alkyl” or “optionally substituted alkyl group” is alkyl or alkyl group optionally substituted with any substituent described herein.

For example, “alkyl” or “alkyl group” is optionally substituted with a halogen atom. “C₁₋₆ alkyl substituted with a halogen atom” refers to “C₁₋₆ alkyl” substituted with the “halogen atom” specified herein, and is also referred to as “halo C₁₋₆ alkyl”, “C₁₋₆ haloalkyl”, “halogeno C₁₋₆ alkyl”, or “C₁₋₆ halogeno alkyl”, or when “C₁₋₆ alkyl” is substituted with hydroxy, “hydroxy C₁₋₆ alkyl” or “C₁₋₆ hydroxyalkyl”. The same applies when substituted with other groups.

As used herein, “alkenyl” or “alkenyl group” refers to a linear or branched unsaturated hydrocarbon group having one or more carbon-carbon double bonds. For example, “C₂₋₆ alkenyl” or “C₂₋₆ alkenyl group” refers to a linear or branched unsaturated hydrocarbon group with 2 to 6 carbon atoms, having one or more carbon-carbon double bonds. Examples of “C₂₋₆ alkenyl group” include “C₂₋₄ alkenyl group” Specific examples of “C₂₋₆ alkenyl group” include, but are not limited to, vinyl groups, 1-propylenyl groups, 2-propylenyl groups, 1-butenyl groups, 2-butenyl groups, 3-butenyl groups, 2-methyl-1-propylenyl groups, 2-methyl-2-propylenyl groups, etc. “Alkenyl” or “alkenyl group” is optionally substituted, just like “alkyl” or “alkyl group”.

As used herein, “alkynyl” or “alkynyl group” refers to a linear or branched unsaturated aliphatic hydrocarbon group with one or more triple bonds. For example, “C₂₋₆ alkynyl” or “C₂₋₆ alkynyl group” refers to a linear or branched unsaturated aliphatic hydrocarbon group with 2 to 6 carbon atoms, having one or more triple bonds. Examples of “C₂₋₆ alkynyl group” include “C₂₋₄ alkynyl group”. Specific examples thereof include, but are not limited to, an ethynyl group, 1-propynyl group, 2-propynyl group, 1-butynyl group, 1-methyl-2-propynyl group, 3-butynyl group, 1-pentynyl group, 1-hexynyl group, etc. “Alkynyl” or “alkynyl group” is optionally substituted, just like “alkyl” or “alkyl group”.

As used herein, “alkylene” or “alkylene group” refers to an alkanediyl group, i.e., linear or branched divalent noncyclic hydrocarbon group. For example, “C₁₋₆ alkylene” refers to alkylene with 1 to 6 carbon atoms, and “C₀₋₃ alkylene” refers to a covalent bond (corresponding to “C₀ alkylene”) or C₁₋₃ alkylene. Examples of alkylene groups include methylene (—CH₂—), ethylene (e.g., —CH₂—CH₂— or —CH(—CH₃)—), propylene (e.g., —CH₂—CH₂—CH₂, —CH(—CH₂—CH₃)—, or CH(—CH₃)—CH₂—), and butylene (e.g., —CH₂—CH₂—CH₂—CH₂—). Examples of “C₁₋₆ alkylene” include “C₁₋₅ alkylene”, “C₁₋₄ alkylene”, and particularly liner C₁₋₄ alkylene. “Alkylene” or “alkylene group” is optionally substituted just like “alkyl” or “alkyl group”, and can be “optionally substituted C₁₋₆ alkylene”.

As used herein, “heteroalkylene” or “heteroalkylene group” refers to a heteroalkanediyl group, i.e., linear or branched divalent noncyclic hydrocarbon group having a heteroatom.

As used herein, “alkenylene” or “alkenylene group” refers to an alkenediyl group, i.e., linear or branched divalent unsaturated hydrocarbon group comprising 1 to 3 double bonds. Specific examples of “C₂₋₇ alkenylene” include a vinylene group, vinylidene group, propenyl group, methylpropenylene group, butenylene group, etc.

As used herein, “alkynylene” or “alkynylene group” refers to an alkynediyl group, i.e., linear or branched divalent unsaturated hydrocarbon group comprising 1 to 3 double bonds. Specific examples of “C₂₋₇ alkynylene” include an ethynylene group, propinylene group, butinylene group, etc.

As used herein, “cycloalkylene” or “cycloalkylene group” refers to a cycloalkanediyl group, i.e., cyclic divalent saturated hydrocarbon group, including those with a partially unsaturated bond and those with a bridged structure. Specific examples of “C₃₋₉ cycloalkylene” include cyclopropylene, cyclobutylene, cyclopentylene, cyclohexylene, cycloheptylene, etc.

“Cycloalkenylene” refers to a cyclic divalent unsaturated hydrocarbon group, including those that have a bridged structure. Specific examples of “C₄₋₆ cycloalkenylene” include cyclobutenylene, cyclopentenylene, cyclohexenylene, etc.

As used herein, “carbocyclyl”, “carbocyclyl group”, “carbocyclic ring”, or “carbocyclic group” can encompass alicyclic groups and aryl groups. “Carbocyclyl”, “carbocyclyl group”, “carbocyclic ring”, or “carbocyclic group” is optionally substituted.

As used herein, “heterocyclyl”, “heterocyclyl group”, “heterocycle”, “heterocyclic group”, “heterocycle”, or “heterocyclic group” can encompass non-aryl heterocyclic groups and heteroaryl groups. “Heterocyclyl”, “heterocyclyl group”, “heterocyclic ring”, “heterocyclic group”, “heterocycle”, or “heterocyclic ring” is optionally substituted.

As used herein, “alicyclic group” refers to a monocyclic or polycyclic monovalent nonaromatic hydrocarbon ring group, including those with a partially unsaturated bond, those with a partially bridged structure, those with a partially spiro form, those that are partially fused, and those with one or more carbonyl structure. An “alicyclic group” can be a “C₃₋₁₀ alicyclic group” with 3 to 10 carbon atoms. “Alicyclic group” encompasses a cycloalkyl group, cycloalkenyl group, and cycloalkynyl group. Examples of “C₃₋₁₀ alicyclic group” include “C₃₋₆ alicyclic group” and “C₅₋₆ alicyclic group”. Specific examples of “C₅₋₆ alicyclic group” include cyclopentyl, cyclohexyl, etc. Specific examples of “C₃₋₆ alicyclic group” include, in addition to the specific examples for the “C₅₋₆ alicyclic group” described above, cyclopropyl, cyclobutyl, etc. Specific examples of “C₃₋₁₀ alicyclic group” include, in addition to the specific examples for the “C₃₋₆ alicyclic group” described above, cycloheptyl, cyclooctyl, cyclononyl, cyclodecyl, and adamantyl.

Specific examples of “C₃₋₁₀ alicyclic group” with a partially bridged structure include, but are not limited to, those with a structure shown below, etc.

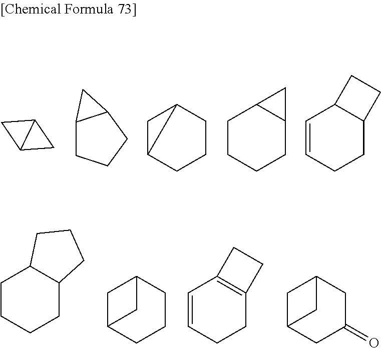

“C₃₋₁₀ alicyclic group” also encompasses compounds fused to an aromatic ring. Specific examples thereof include groups represented by the following, etc.

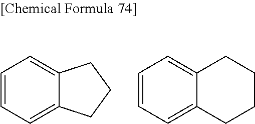

As used herein, “aryl” refers to a monocyclic, bicyclic, tricyclic, or tetracyclic aromatic hydrocarbon group. “C₆₋₁₀ aryl” refers to a monocyclic, bicyclic, tricyclic, or tetracyclic aromatic hydrocarbon group with 6 to 10 carbon atoms. “C₆₋₁₀ aryl” may be fused to the “alicyclic group” or “non-aryl heterocycle” described above at any possible position. Specific examples of “C₆₋₁₀ aryl” include phenyl, 1-naphthyl, 2-naphthyl, etc. Examples of “C₆₋₁₀ aryl” include phenyl. Specific examples of the fused structure include the groups represented by the following, etc.

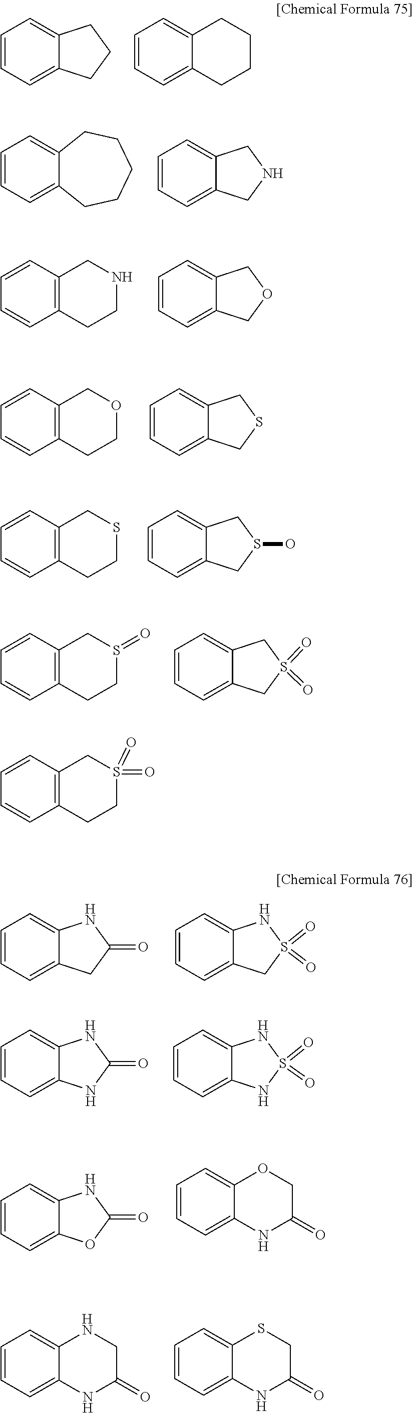

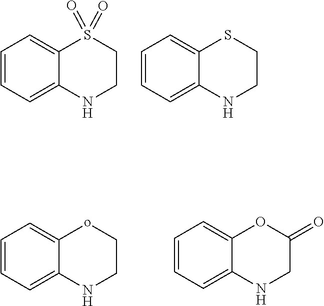

As used herein, “heteroaryl” refers to a monocyclic, bicyclic, tricyclic, or tetracyclic aromatic heterocyclic group comprising an atom independent selected from the group consisting of a nitrogen atom, an oxygen atom, a phosphorous atom, and a sulfur atom. “5- to 10-membered heteroaryl” refers to a monocyclic, bicyclic, tricyclic, or tetracyclic aromatic heterocyclic group comprised of 5 to 10 atoms, comprising 1 to 4 atoms independently selected from the group consisting of a nitrogen atom, an oxygen atom, a phosphorous atom, and a sulfur atom. “5- to 10-membered heteroaryl” may be fused to the “alicyclic group” or “non-aryl heterocycle” described above at any possible position. Examples of “5- to 10-membered heteroaryl” include “5- or 6-membered heteroaryl”, “6- to 10-membered heteroaryl”, and “9- or 10-membered heteroaryl”. Specific examples of “5- or 6-membered heteroaryl” include furyl, thienyl, pyrrolyl, pyrazolyl, oxazolyl, thiazolyl, imidazolyl, isooxazolyl, pyridyl, pyrazinyl, pyrimidinyl, and pyridazinyl. Specific examples of “6- to 10-membered heteroaryl” include pyridyl, pyrazinyl, pyrimidinyl, pyridazinyl, quinoxalyl, triazolopyridyl, etc. Specific examples of “5- to 10-membered heteroaryl” include the specific examples for the “6- to 10-membered heteroaryl” and “5- to 6-membered heteroaryl” described above.

Specific examples of “9- or 10-membered heteroaryl” include, but are not limited to, those with a structure shown below, etc.

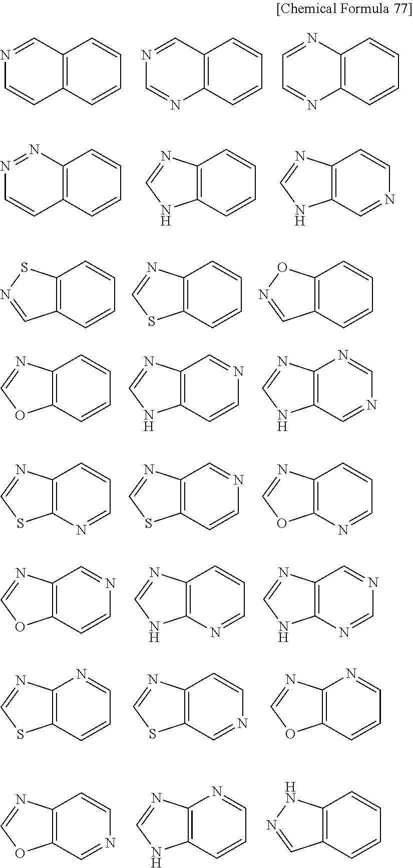

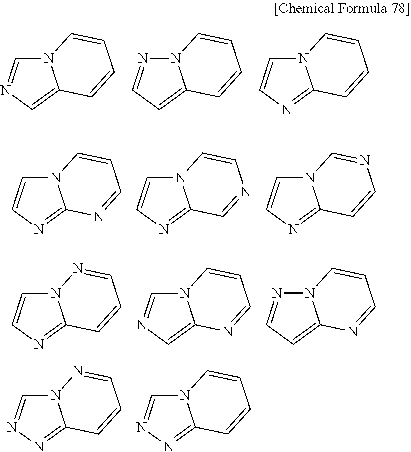

The “5- or 6-membered heteroaryl” or “5- to 10-membered heteroaryl” may form a fused structure with a C₅₋₁₀ alicyclic group, or a fused structure with a 5- to 10-membered non-aryl heterocycle. Specific examples thereof include the groups represented by the following, etc.

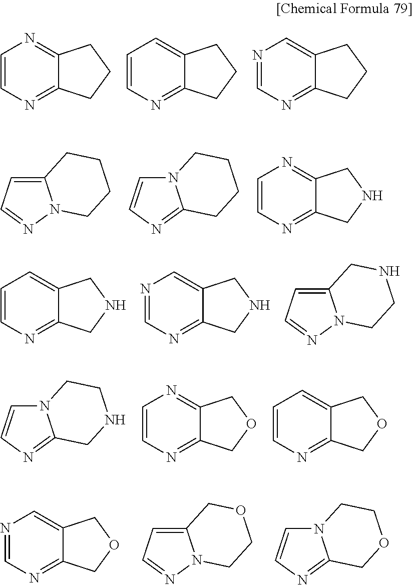

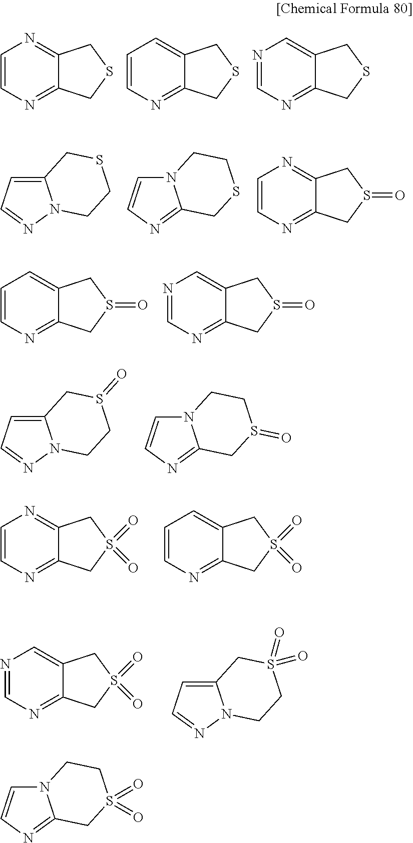

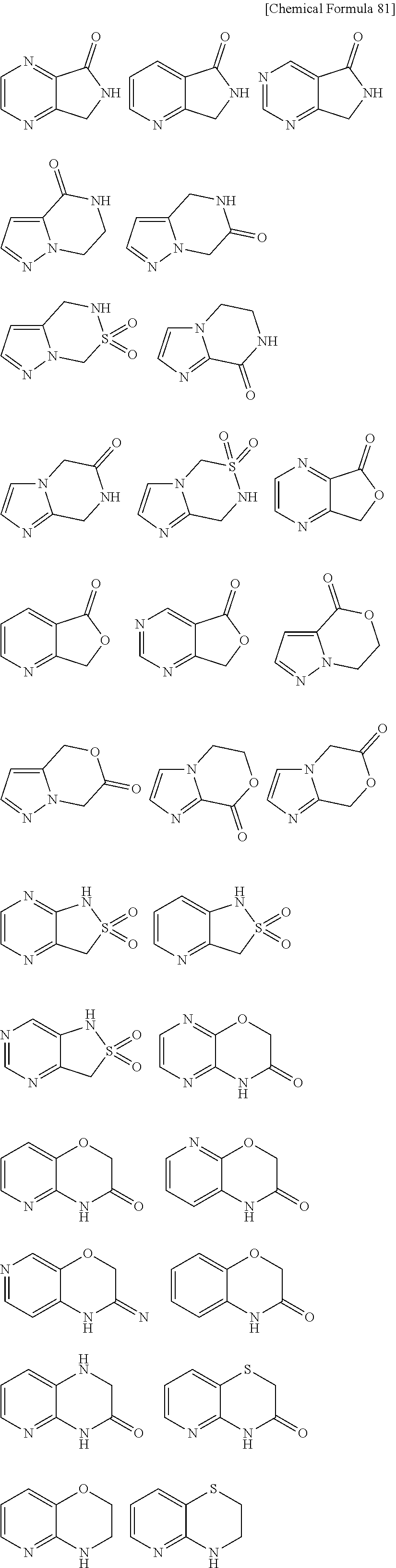

As used herein, “N-containing heteroaryl” refers to heteroaryl having a nitrogen atom. The heteroaryl moiety is defined the same as the “heteroaryl” described above. Specific examples of “5-membered N-containing heteroaryl” include pyrrolyl, pyrazolyl, oxazolyl, thiazolyl, imidazolyl, and isooxazolyl.

As used herein, “non-aryl heterocyclic group” refers to a monocyclic, bicyclic, tricyclic, or tetracyclic nonaromatic heterocycle comprising the same or different heteroatoms independently selected from the group consisting of a nitrogen atom, an oxygen atom, and a sulfur atom, other than a carbon atom, including those with a partially unsaturated bond, those with a partially bridged structure, and/or those with a partially spiro form. “4- to 10-membered non-aryl heterocyclic group” refers to a monocyclic, bicyclic, tricyclic, or tetracyclic nonaromatic heterocycle comprised of 4 to 10 atoms, comprising the same or different 1 to 2 heteroatoms independently selected from the group consisting of a nitrogen atom, an oxygen atom, and a sulfur atom, other than a carbon atom, including those with a partially unsaturated bond, those with a partially bridged structure, and/or those with a partially spiro form. “4- to 10-membered non-aryl heterocyclic group” is preferably a “4- to 6-membered non-aryl heterocyclic group”. Specific examples of “4- to 6-membered non-aryl heterocyclic group” include azetidinyl, pyrrolidinyl, piperidyl, piperazinyl, morpholinyl, tetrahydrofuranyl, tetrahydropyranyl, etc. In particular, azetidinyl, pyrrolidinyl, piperidyl, morpholinyl, and oxetanyl are preferred. A non-aryl heterocycle may form a fused ring with aryl or heteroaryl. For example, those fused to C₆₋₁₀ aryl or 5- or 6-membered heteroaryl is also encompassed by non-aryl heterocycle. Further, the non-aryl heterocycle may be comprised by including one or more carbonyl, thiocarbonyl, sulfinyl, or sulfonyl. The non-aryl heterocycles also encompass, for example, lactam, thiolactam, lactone, thiolactone, cyclic imide, cyclic carbamate, cyclic thiocarbamate, and other cyclic groups. In this regard, oxygen atoms of carbonyl, sulfinyl, and sulfonyl and sulfur atoms of thiocarbonyl are not included in the number of 4 to 10 members (size of ring) or in the number of heteroatoms constituting a ring. Examples of “4- to 10-membered non-aryl heterocycle” include “4- to 6-membered non-aryl heterocycle”. Specific examples of “4- to 6-membered non-aryl heterocycle” include azetidine, pyrrolidine, piperidine, piperazine, morpholine, homopiperidine, oxetane, tetrahydrofuran, tetrahydropyran, etc. Specific examples of “4- to 10-membered non-aryl heterocycle” include, in addition to the specific examples for the “4- to 6-membered non-aryl heterocycle” described above, those with the structure shown below, etc.

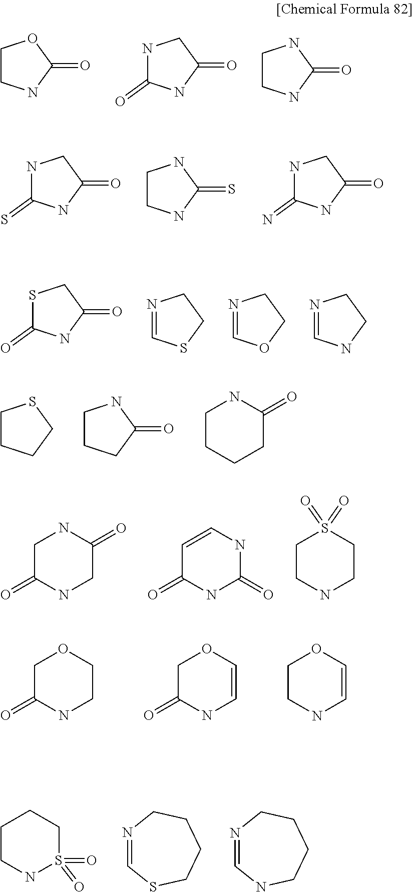

Specific examples of “4- to 10-membered non-aryl heterocycle” having a partially bridged and/or spiro structure include, but are not limited to, those with a structure shown below, etc.

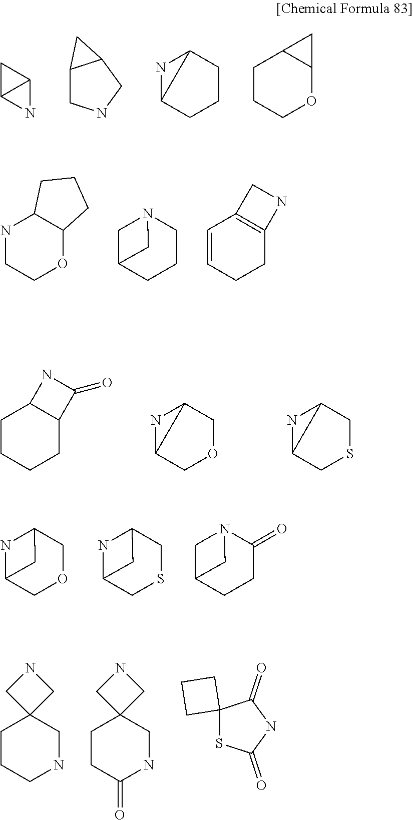

Specific examples of “4-membered non-aryl heterocycle” having a partially unsaturated bond include, but are not limited to, those with a structure shown below, etc.

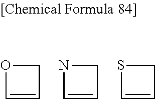

Specific examples “5-membered non-aryl heterocycle” having a partially unsaturated bond include, but are not limited to, those with a structure shown below, etc.

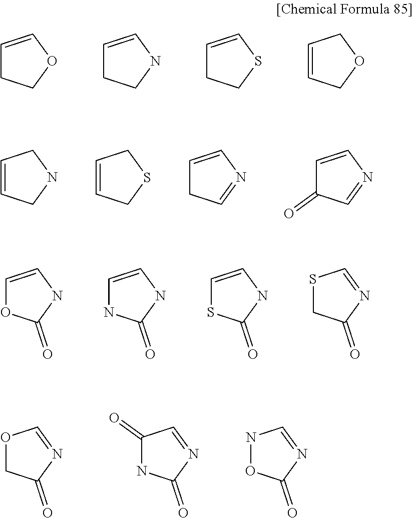

Specific examples of “5-membered non-aryl heterocycle” having a partially bridged structure include, but are not limited to, those with a structure shown below, etc.

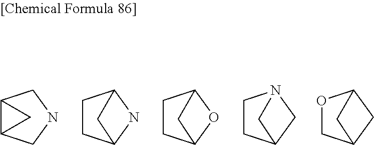

Specific examples of “5-membered non-aryl heterocycle” comprising carbonyl, thiocarbonyl, etc. include, but are not limited to, those with a structure shown below, etc.

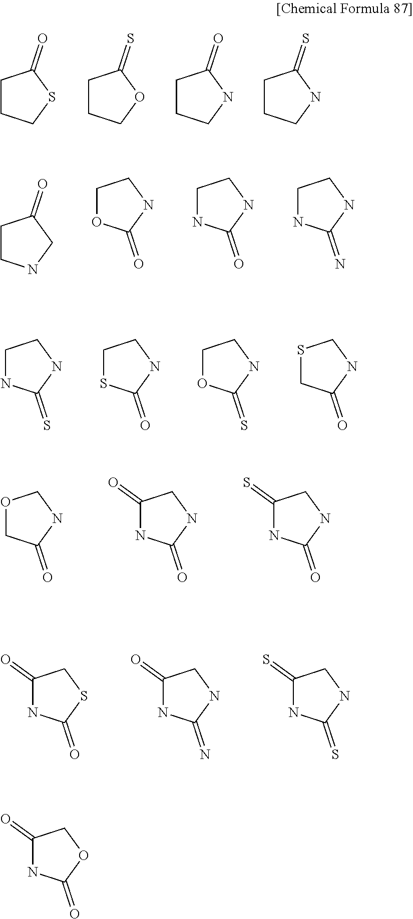

Specific examples of “6-membered non-aryl heterocycle” having a partially unsaturated bond include, but are not limited to, those with a structure shown below, etc.

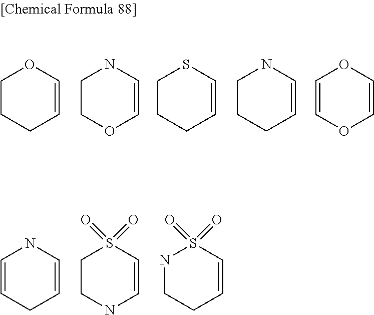

Specific examples of “6-membered non-aryl heterocycle” having a partially bridged structure include, but are not limited to, those with a structure shown below, etc.

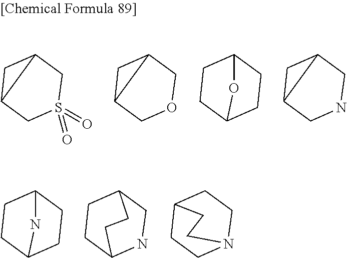

“Alkoxy” or “alkoxy group” refers to “alkyloxy”, and the “alkyl” moiety is defined the same as the “alkyl” described above. “Alkoxy” or “alkoxy group” can be “C₁₋₆ alkoxy” or “C₁₋₆ alkoxy group”. Examples of “C₁₋₆ alkoxy” include “C₁₋₄ alkoxy” and “C₁₋₃ alkoxy”. Specific examples of “C₁₋₃ alkoxy” include methoxy, ethoxy, propoxy, 1-methylethoxy, etc. Specific examples of “C₁₋₄ alkoxy” include, in addition to the specific examples for the “C₁₋₃ alkyl” described above, butoxy, 1,1-dimethylethoxy, 1-methylpropoxy, 2-methylpropoxy, etc. Specific examples of “C₁₋₆ alkoxy” include, in addition to the specific examples for the “C₁₋₄ alkyl” described above, pentyloxy, 1,1-dimethylpropoxy, 1,2-dimethylpropoxy, 1-methylbutoxy, 2-methylbutoxy, 4-methylpentyloxy, 3-methylpentyloxy, 2-methylpentyloxy, 1-methylpentyloxy, hexyloxy, etc.

“Alicyclic oxy” or “alicyclic oxy group” refers to an (alicyclic group)-O— group, and the alicylic moiety is defined the same as an alicyclic group. A description such as “—O-cycloalkyl” refers to an “(alicyclic group)-O— group”. “Alicyclic oxy” or “alicyclic oxy group” can be “C₃₋₇ alicyclic oxy” or “C₃₋₇ alicyclic oxy group”. “C₃₋₇ alicyclic oxy group” encompasses “C₃₋₇ cycloalkoxy group”. “Cycloalkoxy group” refers to “cycloalkyloxy”, and the “cycloalkyl” moiety is defined the same as the “cycloalkyl” described above. Specific examples of “C₃₋₆ alicyclic oxy group” include a cyclopropoxy group, cyclobutoxy group, cyclopentoxy group, cyclohexoxy group, etc.

The C₆₋₁₀ aryl moiety of “C₆₋₁₀ aryloxy group” is defined the same as C₆₋₁₀ aryl described above. Preferred examples of “C₆₋₁₀ aryloxy group” include “C₆ or C₁₀ aryloxy group”. Specific examples of “C₆₋₁₀ aryloxy group” include, but are not limited to, phenoxy group, 1-naphthyloxy group, 2-naphthyloxy group, etc.

The heterocyclyl moiety of “heterocyclyloxy group” is defined the same as the “heterocyclyl” described above. Examples of “heterocyclyloxy group” include, but are not limited to, a heteroaryloxy group, non-aryl heterocyclyl oxy group, etc.

The 5- or 6-membered heteroaryl moiety of “5- or 6-membered heteroaryloxy group” is defined the same as the “5-membered heteroaryl” or “6-membered heteroaryl” described above. Specific examples of “5- or 6-membered heteroaryloxy group” include, but are not limited to, pyrazoyloxy group, triazoyloxy group, thiazoyloxy group, thiadiazoyloxy group, pyridyloxy group, pyridazoyloxy group, etc.

The 4- to 10-membered non-aryl heterocycle moiety of “4- to 10-membered non-aryl heterocyclyl oxy group” is defined the same as the “4- to 10-membered non-aryl heterocycle” described above. Examples of “4- to 10-membered non-aryl heterocyclyl oxy group” include “4- to 6-membered non-aryl heterocyclyl oxy group”. Specific examples of “4- to 10-membered non-aryl heterocyclyl oxy group” include, but are not limited to, a tetrahydrofuranyloxy group, tetrahydropyranyloxy group, azetidinyloxy group, pyrrolidinyloxy group, piperidinyloxy group, etc.

The C₁₋₆ alkyl moiety of “C₁₋₆ alkylthio group” is defined the same as the C₁₋₆ alkyl described above. “C₁₋₆ alkylthio group” can be a “C₁₋₄ alkylthio group”, or a “C₁₋₃ alkylthio group”. Specific examples of “C₁₋₆ alkylthio group” include, but are not limited to, a methylthio group, ethylthio group, propylthio group, butylthio group, isopropylthio group, isobutylthio group, tert-butylthio group, sec-butylthio group, isopentylthio group, neopentylthio group, tert-pentylthio group, 1,2-dimethylpropylthio group, etc.

“C₃₋₁₀ alicyclic thio” or “C₃₋₁₀ alicyclic thio group” refers to a (C₃₋₁₀ alicyclic group)-S— group, and the C₃₋₁₀ alicyclic moiety is defined the same as the C₃₋₁₀ alicyclic group described above. “C₃₋₁₀ alicyclic thio group” is preferably a “C₃₋₆ alicyclic thio group”. Specific examples of “C₃₋₆ alicyclic thio group” include, but are not limited to, a cyclopropylthio group, cyclobutylthio group, cyclopentylthio group, cyclohexylthio group, etc.

The C₆₋₁₀ aryl moiety of “C₆₋₁₀ arylthio” or “C₆₋₁₀ arylthio group” is defined the same as the C₆₋₁₀ aryl described above. “C₆₋₁₀ arylthio group” is preferably a “C₆ or C₁₀ arylthio group”. Specific examples of “C₆₋₁₀ arylthio group” include, but are not limited to, a phenylthio group, 1-naphthylthio group, 2-naphthylthio group, etc.

The 5- or 6-membered heteroaryl moiety of “5- or 6-membered heteroarylthio” or “5- or 6-membered heteroarylthio group” is defined the same as the “5-membered heteroaryl” or “6-membered heteroaryl” described above. Specific examples of “5- or 6-membered heteroarylthio group” include, but are not limited to, a pyrazoylthio group, triazoylthio group, thiazoylthio group, thiadiazoylthio group, pyridylthio group, pyridazoylthio group, etc.

The 4- to 10-membered non-aryl heterocycle moiety of “4- to 10-membered non-aryl heterocyclyl thio” or “4- to 10-membered non-aryl heterocyclyl thio group” is defined the same as the “4- to 10-membered non-aryl heterocycle” described above. “4- to 10-membered non-aryl heterocyclyl thio group” is preferably a “4- to 6-membered non-aryl heterocyclyl thio group”. Specific examples of “4- to 10-membered non-aryl heterocyclyl thio group” include, but are not limited to, a tetrahydropyranylthio group, piperidinylthio group, etc.

“C₁₋₆ alkylcarbonyl” or “C₁₋₆ alkylcarbonyl group” refers to a carbonyl group substituted with the “C₁₋₆ alkyl group” described above. “C₁₋₆ alkylcarbonyl group” is preferably a “C₁₋₄ alkylcarbonyl group”. Specific examples of “C₁₋₆ alkylcarbonyl group” include, but are not limited to, an acetyl group, propionyl group, butyryl group, etc. “C₂₋₇ alkanoyl group” indicates a group in which the “C₁₋₆ alkyl group” described above is attached to a carbon atom of a carbonyl group. Examples thereof include an acetyl group, propionyl group, butyryl group, isobutyryl group, pivaloyl group, valeryl group, isovaleryl group, hexanoyl group, heptanoyl group, etc.

“C₃₋₁₀ alicyclic carbonyl” or “C₃₋₁₀ alicyclic carbonyl group” refers to a carbonyl group substituted with the “C₃-10 alicyclic group” described above. “C₃₋₁₀ alicyclic carbonyl group” is preferably a “C₃₋₆ alicyclic carbonyl group”. Specific examples of “C₃₋₁₀ alicyclic carbonyl group” include, but are not limited to, a cyclopropylcarbonyl group, cyclopentylcarbonyl group, etc.

“C₆₋₁₀ arylcarbonyl” or “C₆₋₁₀ arylcarbonyl group” refers to a carbonyl group substituted with the “C₆₋₁₀ aryl” described above. “C₆₋₁₀ arylcarbonyl group” is preferably a “C₆ or C₁₀ arylcarbonyl group”. Specific examples of “C₆₋₁₀ arylcarbonyl group” include, but are not limited to, a benzoyl group, 1-naphthylcarbonyl group, 2-naphthylcarbonyl group, etc.

“5- or 6-membered heteroarylcarbonyl” or “5- or 6-membered heteroarylcarbonyl group” refers to a carbonyl group substituted with the “5- or 6-membered heteroaryl” described above. Specific examples of “5- or 6-membered heteroarylcarbonyl group” include, but are not limited to, a pyrazoylcarbonyl group, triazoylcarbonyl group, thiazoylcarbonyl group, thiadiazoylcarbonyl group, pyridylcarbonyl group, pyridazoylcarbonyl group, etc.

“4- to 10-membered non-aryl heterocyclyl carbonyl” or “4- to 10-membered non-aryl heterocyclyl carbonyl group” refers to a carbonyl group substituted with the “4- to 10-membered non-aryl heterocycle” described above. “4- to 10-membered non-aryl heterocyclyl carbonyl group” is preferably a “4- to 6-membered non-aryl heterocyclyl carbonyl group”. Specific examples of “4- to 10-membered non-aryl heterocyclyl carbonyl group” include, but are not limited to, an azetidinylcarbonyl group, pyrrolidinylcarbonyl group, piperidinylcarbonyl group, morpholinylcarbonyl group, etc.

“C₁₋₆ alkylsulfonyl” or “C₁₋₆ alkylsulfonyl group” refers to a sulfonyl group substituted with the “C₁₋₆ alkyl group” described above. “C₁₋₆ alkylsulfonyl group” is preferably a “C₁₋₄ alkylsulfonyl group”. Specific examples of “C₁₋₆ alkylsulfonyl group” include, but are not limited to, a methylsulfonyl group, propionylsulfonyl group, butyrylsulfonyl group, etc.

“C₃₋₁₀ alicyclic sulfonyl” or “C₃₋₁₀ alicyclic sulfonyl group” refers to a sulfonyl group substituted with the “C₃₋₁₀ alicyclic group” described above. “C₃₋₁₀ alicyclic sulfonyl group” is preferably a “C₃₋₆ alicyclic sulfonyl group”. Specific examples of “C₃₋₁₀ alicyclic sulfonyl group” include, but are not limited to, a cyclopropylsulfonyl group, cyclobutylsulfonyl group, cyclopentylsulfonyl group, cyclohexylsulfonyl group, etc.

“C₆₋₁₀ arylsulfonyl” or “C₆₋₁₀ arylsulfonyl group” refers to a sulfonyl group substituted with the “C₆₋₁₀ aryl” described above. “C₆₋₁₀ arylsulfonyl group” is preferably a “C₆ or C₁₀ arylsulfonyl group”. Specific examples of “C₆₋₁₀ arylsulfonyl group” include, but are not limited to, a phenylsulfonyl group, 1-naphthylsulfonyl group, 2-naphthylsulfonyl group, etc.

“5- or 6-membered heteroarylsulfonyl” or “5- or 6-membered heteroarylsulfonyl group” refers to a sulfonyl group substituted with the “5- or 6-membered heteroaryl” described above. Specific examples of “5- or 6-membered heteroarylsulfonyl group” include a pyrazoylsulfonyl group, triazoylsulfonyl group, thiazoylsulfonyl group, thiadiazoylsulfonyl group, pyridylsulfonyl group, pyridazoylsulfonyl group, etc.

As used herein, “amino” or “amino group” refers to an —NH₂ group. An amino group may be substituted with any substituent disclosed herein. Examples thereof include a C₂₋₇ alkanoylamino group, C₁₋₆ alkylsulfonylamino group, C₃₋₇ cycloalkylsulfonylamino group, phenylsulfonylamino group, C₁₋₆ alkylamino group, etc.

As used herein, “amide” or “amide group” refers to an aminocarbonyl group (—C(═O)NH₂) or a carbonylamino group (R—C(═O)—NH—) (wherein R can be alkyl, alkenyl, alkynyl, cycloalkyl, aryl, or heteroaryl). “Substituted amide” refers to a group with an amino moiety of amide substituted. —C(═O)—NH₂ is also referred to as a carbamoyl group. A carbamoyl group can have a substituted amino moiety.

As used herein, “ester group” can be an alkoxycarbonyl group or an alkylcarbonyloxy group (alkanoyloxy group). Examples thereof include a C₁₋₄ alkoxycarbonyl group and C₁₋₄ alkanoyloxy group.

As used herein, “urea group” refers to —NH—C(═O)—NH₂, and “substituted urea group” refers to a group with an amine moiety substituted.

As used herein, “halogeno C₁₋₆ alkylsulfonyloxy group” refers to a sulfonyloxy group having halogenated C₁₋₆ alkyl.

As used herein, “arylene” or “arylene group” refers to an aryldiyl group, i.e., divalent aryl group. For example, “C₆₋₁₀ arylene” can be phenylene, and can attach to other groups at positions 1, 6, 1, 5, 1, 4, 1, 3, or 1, 2. “Arylene” or “arylene group” is optionally substituted and can be “optionally substituted C₆₋₁₀ arylene”.

As used herein, “heteroarylene” or “heteroarylene group” refers to a heteroaryldiyl group, i.e., divalent heteroaryl group. For example, “C₆₋₁₀ heteroarylene” can be pyridylene, and can attach to other groups at any two positions. “Heteroarylene” or “heteroarylene group” is optionally substituted and can be “optionally substituted C₆₋₁₀ heteroarylene”.

As used herein, “substituted guanidino” means that any amino group or imino group of a guanidino group (—NH—C(═NH)—NH₂) is substituted with any group.

As used herein, “Degron” refers to a moiety that attaches to an E3 ubiquitin ligase.

As used herein, “E3 ubiquitin ligase” refers to cerebron (CRBRN) or von Hippel-Lindau (VHL).

As used herein, “C₁₋₄ sulfoalkyl” refers to a linear or branched sulfur atom-containing saturated hydrocarbon group with 1 to 4 carbon atoms.

(Pharmaceutical composition) The present disclosure provides a pharmaceutical composition for use in treating and/or preventing cancer, comprising a CBP/P300 inhibitor

In one embodiment, the cancer is SWI/SNF complex dysfunction cancer.

In one embodiment, the SWI/SNF complex dysfunction cancer is BAF complex dysfunction cancer.

In one embodiment, the BAF complex dysfunction cancer is SMARC deficient cancer, SS18-SSX fusion cancer, or ARID deficient cancer.

In one embodiment, the cancer is SMARC deficient cancer.

In one embodiment, the SMARC deficient cancer is SMARCB1 deficient cancer.

In one embodiment, the SMARCB1 deficient cancer is malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, or mesothelioma.

In one embodiment, the SMARCB1 deficient cancer is malignant rhabdoid tumor, epithelioid sarcoma, or atypical teratoid/rhabdoid tumor.

In one embodiment, the SMARCB1 deficient cancer is malignant rhabdoid tumor.

In one embodiment, the SMARC deficient cancer is SMARCA2 deficient cancer.

In one embodiment, the SMARCA2 deficient cancer is pulmonary adenocarcinoma, large cell lung carcinoma, lung neuroendocrine tumor, esophageal cancer, gastroesophageal junction cancer, or malignant rhabdoid tumor.

In one embodiment, the SMARCA2 deficient cancer is pulmonary adenocarcinoma.

In one embodiment, the SMARC deficient cancer is SMARCA4 deficient cancer.

In one embodiment, the SMARCA4 deficient cancer is pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, or thoracic cavity sarcoma.

In one embodiment, the SMARCA4 deficient cancer is pulmonary adenocarcinoma.

In one embodiment, the SMARC deficient cancer is SMARCA2/A4 deficient cancer.

In one embodiment, the SMARCA2/A4 deficient cancer is pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, or small cell lung cancer.

In one embodiment, the SMARCA2/A4 deficient cancer is pulmonary adenocarcinoma.

In one embodiment, the cancer is ARID deficient cancer.

In one embodiment, the ARID deficient cancer is ARID1A deficient cancer.

In one embodiment, the ARID1A deficient cancer is ovarian cancer, colon cancer, uterine cancer, neuroblastoma, bladder cancer, or gastric cancer.

In one embodiment, the ARID1A deficient cancer is ovarian cancer.

In one embodiment, the ARID deficient cancer is ARID1B deficient cancer.

In one embodiment, the ARID1B deficient cancer is ovarian cancer, colon cancer, uterine cancer, neuroblastoma, bladder cancer, or gastric cancer.

In one embodiment, the ARID1B deficient cancer is ovarian cancer.

In one embodiment, the ARID deficient cancer is ARID1A/1B deficient cancer.

In one embodiment, the ARID1A/1B deficient cancer is ovarian cancer, colon cancer, uterine cancer, neuroblastoma, bladder cancer, or gastric cancer.

In one embodiment, the ARID1A/1B deficient cancer is ovarian cancer.

In one embodiment, the cancer is SS18-SSX fusion cancer.

In one embodiment, the SS18-SSX fusion cancer is synovial sarcoma or Ewing's sarcoma.

In one embodiment, the SS18-SSX fusion cancer is synovial sarcoma.

In one embodiment, the CBP/P300 inhibitor is a HAT inhibitor, a BRD inhibitor, an antisense nucleic acid for a transcriptional product of a gene encoding CBP or P300, a ribozyme for a transcriptional product of a gene encoding CBP or P300, a nucleic acid having RNAi activity for a transcriptional product of a gene encoding CBP or P300, or a precursor thereof.

In one embodiment, the CBP/P300 inhibitor is a HAT inhibitor or a BRD inhibitor.

In one embodiment, the CBP/P300 inhibitor is a HAT inhibitor.

In one embodiment, activity of the HAT inhibitor inhibits histone acetyltransferase (HAT) activity of CBP and/or P300 by 50% or more at 20 μM.

In one embodiment, activity of the HAT inhibitor inhibits histone acetyltransferase (HAT) activity of CBP and/or P300 by 80% or more at 20 μM.

In one embodiment, the CBP/P300 inhibitor is a nucleic acid or a low molecular weight compound.

In one embodiment, the HAT inhibitor is a low molecular weight compound.

In one embodiment, the low molecular weight compound is a compound listed below.

While the preferred variables in the compound of the present disclosure represented by formula (1) are described below, the technical scope of the present disclosure is not limited to the scope of the compounds described below.

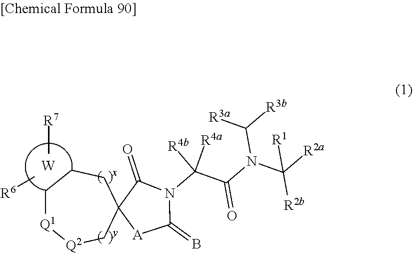

(1-1) Q¹ - - - Q² is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or C(R¹⁰)═C(R¹⁴)—;
(1-1-1) Q¹ - - - Q² is —C(R¹⁰)₂—C(R¹⁴)₂—.
(1-2) A is —NR⁸—, —O—, or —S—;
(1-2-1) A is —NR⁸—;
(1-2-2) A is —O—.
(1-3) B is O or NH;
(1-3-1) B is O.
(1-4) W is arylene or heteroarylene;
(1-4-1) W is arylene;
(1-4-2) W is phenylene.
(1-5) R¹ is carbocyclyl or heterocyclyl;
(1-5-1) R¹ is carbocyclyl;
(1-5-2) R¹ is optionally substituted phenyl.
(1-6) R^{2a} and R^{2b} are each independently a hydrogen atom, a deuterium atom, C₁₋₆ alkyl, C₁₋₆ alkenyl, or C₁₋₆ alkynyl;
(1-6-1) R^{2a} and R^{2b} are hydrogen atoms.
(1-7) R^{3a} is a hydrogen atom, C(O)NH₂, C₁₋₆ alkyl, C₁₋₆ alkenyl or C₁₋₆ alkynylaryl, cycloalkyl, or heterocyclyl;
(1-7-1) R^{3a} is C₁₋₆ alkyl.
(1-8) R^{3b} is C₁₋₆ alkyl, aryl, cycloalkyl, or heterocyclyl;
(1-8-1) R^{3b} is C₁₋₆ alkyl.
(1-9) R^{4a} and R^{4b} are each independently a hydrogen atom, a deuterium atom, C₁₋₆ alkyl, C₁₋₆ alkenyl, or C₁₋₆ alkynyl;
(1-9-1) R^{4a} and R^{4b} are hydrogen atoms.
(1-10) R⁶ and R⁷ are each independently a hydrogen atom, a halogen atom, —OH, —CN, —CO₂H, C₁₋₆ alkyl, C₁₋₆ alkenyl, C₁₋₆ alkynyl, alkoxy, haloalkoxy, alkoxyalkyl, haloalkoxyalkyl, hydroxyalkyl, hydroxyalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, NHC(═O)NR¹²S(O)₂OR¹², —NHC(═O)NR¹²S(O)₂N(R¹²)₂, —NHC(═S)N(R¹²)₂, —NHC(═N—C≡N) NR¹², —NHC(═N—C≡N) SR¹², or —NHS(O)ₘR¹²;
(1-10-1) R⁶ is heterocyclyl, and R⁷ is H;
(1-10-2) R⁶ is optionally substituted pyrazolyl, and R⁷ is H;
(1-10-3) R⁶ is —NHC(O)NHCH₃, and R⁷ is H.
(1-11) R⁸ and R⁹ are each independently a hydrogen atom, C₁₋₆ alkyl, C₁₋₆ alkenyl, or C₁₋₆ alkynyl;
(1-11-1) R⁸ and R⁹ are each hydrogen atoms.
(1-12) R¹⁰ are, for each instance, each independently a hydrogen atom, —OH, a halogen atom, —CN, —C₀₂R¹², —C(═O)NHR¹³, —NHR¹², C₁₋₆ alkyl, C₁₋₆ alkenyl, C₁₋₆ alkynyl, or alkoxy; or wherein two R¹⁰ together may form oxo or ═N—OR¹¹;
(1-12-1) R¹⁰ are, for each instance, each independently a hydrogen atom, —OH, or a halogen atom.
(1-13) R¹¹ and R¹³ are each independently a hydrogen atom, —OH, C₁₋₆ alkyl, C₁₋₆ alkenyl, or C₁₋₆ alkynyl.
(1-14) R¹² are, for each instance, each independently a hydrogen atom, C₁₋₆ alkyl, C₁₋₆ alkenyl, C₁₋₆ alkynylaryl, cycloalkyl, or heterocyclyl.
(1-15) R¹⁴ are, for each instance, each independently a hydrogen atom, C₁₋₆ alkyl, C₁₋₆ alkenyl, or C₁₋₆ alkynyl;
(1-15) R¹⁴ is a hydrogen atom.
(1-16) m are, for each instance, each independently 0, 1, or 2.
(1-17) x and y are each independently 0 or 1, wherein x and y are chosen so that the sum of x+y is 0 or 1;
(1-17-1) x is 0, and y is 0.

In formula (1), Q¹- - - Q² can be 1-1-1, B can be 1-3-1, R^{2a} and R^{2b} can be 1-6-1, R^{3a} can be 1-7-1, R^{3b} can be 1-8-1, R^{4a} and R^{4b} can be 1-9-1, R⁸ and R⁹ can be 1-11-1, R¹⁴ can be 1-15-1, x and y can be 1-17-1, and A, W, R¹, R⁶, and R⁷ can be the following.

TABLE 43

| A | W | R¹ | R⁶, R⁷ |
|---|---|---|---|
| 1-2-1 | 1-4-1 | 1-5-1 | 1-10-1 |
| 1-2-2 | 1-4-1 | 1-5-1 | 1-10-1 |
| 1-2-1 | 1-4-2 | 1-5-1 | 1-10-1 |
| 1-2-2 | 1-4-2 | 1-5-1 | 1-10-1 |
| 1-2-1 | 1-4-1 | 1-5-2 | 1-10-1 |
| 1-2-2 | 1-4-1 | 1-5-2 | 1-10-1 |
| 1-2-1 | 1-4-2 | 1-5-2 | 1-10-1 |
| 1-2-2 | 1-4-2 | 1-5-2 | 1-10-1 |
| 1-2-1 | 1-4-1 | 1-5-1 | 1-10-2 |
| 1-2-2 | 1-4-1 | 1-5-1 | 1-10-2 |
| 1-2-1 | 1-4-2 | 1-5-1 | 1-10-2 |
| 1-2-2 | 1-4-2 | 1-5-1 | 1-10-2 |
| 1-2-1 | 1-4-1 | 1-5-2 | 1-10-2 |
| 1-2-2 | 1-4-1 | 1-5-2 | 1-10-2 |
| 1-2-1 | 1-4-2 | 1-5-2 | 1-10-2 |
| 1-2-2 | 1-4-2 | 1-5-2 | 1-10-2 |
| 1-2-1 | 1-4-1 | 1-5-1 | 1-10-3 |
| 1-2-2 | 1-4-1 | 1-5-1 | 1-10-3 |
| 1-2-1 | 1-4-2 | 1-5-1 | 1-10-3 |
| 1-2-2 | 1-4-2 | 1-5-1 | 1-10-3 |
| 1-2-1 | 1-4-1 | 1-5-2 | 1-10-3 |
| 1-2-2 | 1-4-1 | 1-5-2 | 1-10-3 |
| 1-2-1 | 1-4-2 | 1-5-2 | 1-10-3 |
| 1-2-2 | 1-4-2 | 1-5-2 | 1-10-3 |

While the preferred variables in the compound of the present disclosure represented by formula (2) are described below, the technical scope of the present disclosure is not limited to the scope of the compounds described below.

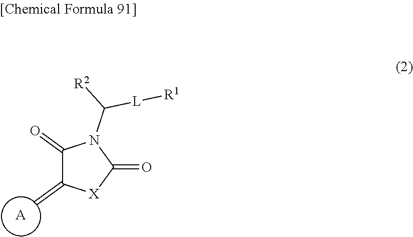

(2-1) A is carbocyclyl or heterocyclyl with a 6-, 7-, or 8-membered ring, and heterocyclyl is comprised of a carbon atom and one or more heteroatoms selected from O and S;
(2-1-1) A is

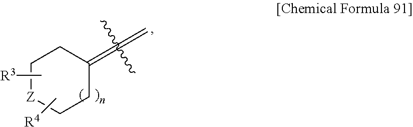

Z is —(CR⁵)(R⁶)—, —O—, —S—, or —S(O)₂—,
R³, R⁴, R⁵, and R⁶ are each independently selected from H, D, hydroxyl, halo, carboxyl, nitrile, C₁₋₆ alkyl, alkoxy, alkoxyalkyl, haloalkyl, haloalkoxy, haloalkoxyalkyl, heteroaryl, and alkoxycarbonyl; or R³ and R⁴ or R⁵ and R⁶ together form a heterocycle or a carbocyclic ring, R³ and R⁴ attach to the same or different carbon atom, and n is 1, 2, or 3;
(2-1-2) A is one of

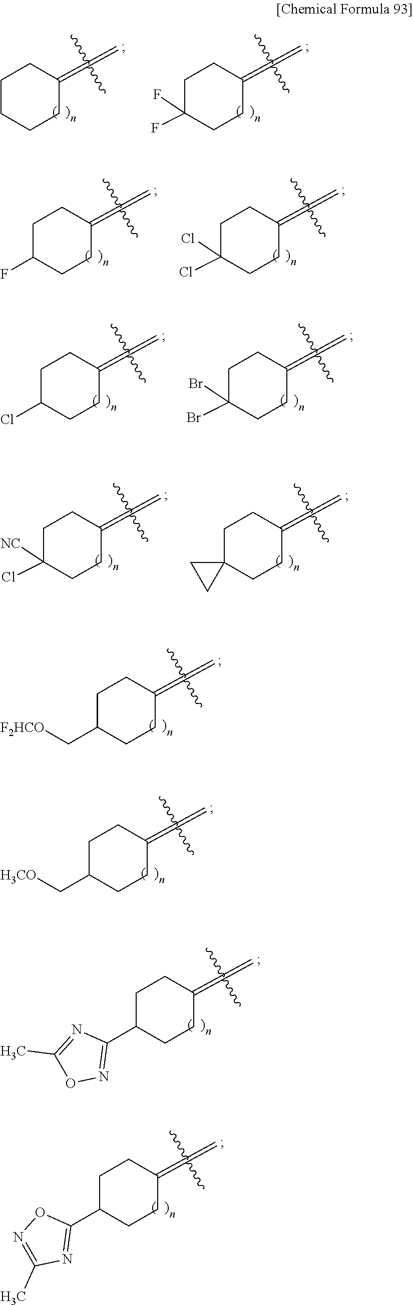

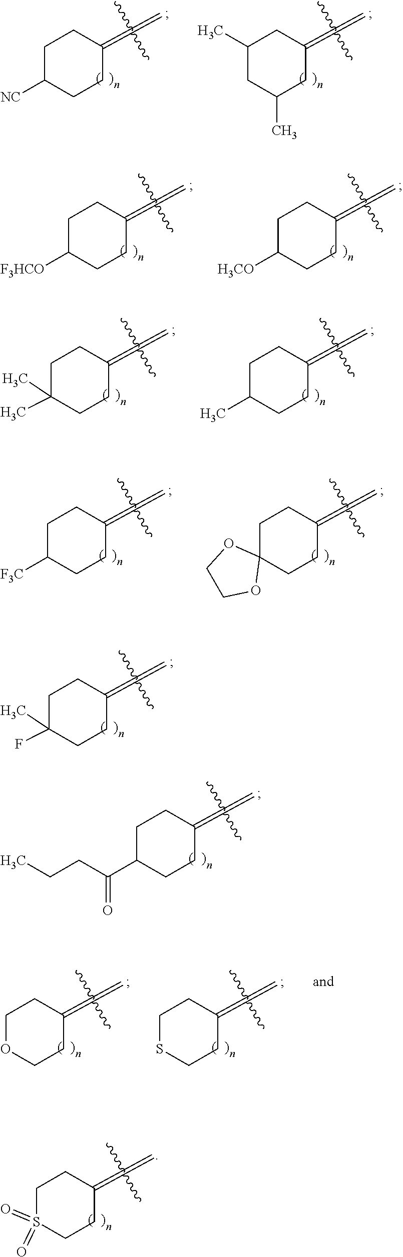

(2-2) X is —S— or —NH—;
(2-2-1) X is —S—.
(2-3) L is a direct bond or a linker;
(2-3-1) L is alkylene, alkenylene, alkynylene, carbonyl, or amidyl (—C(═O)NH— or —NHC(═O)—).
(2-4) R¹ is aryl, heteroaryl, or cycloalkyl;
(2-4-1) R¹ is optionally substituted phenyl, optionally substituted pyrazolyl, optionally substituted piperazinyl, optionally substituted pyridyl, optionally substituted pyrazyl, optionally substituted pyridazinyl, optionally substituted pyrimidinyl, or optionally substituted thiazolyl, wherein the “optionally substituted” includes a fused ring.
(2-4-2) R¹ is selected from

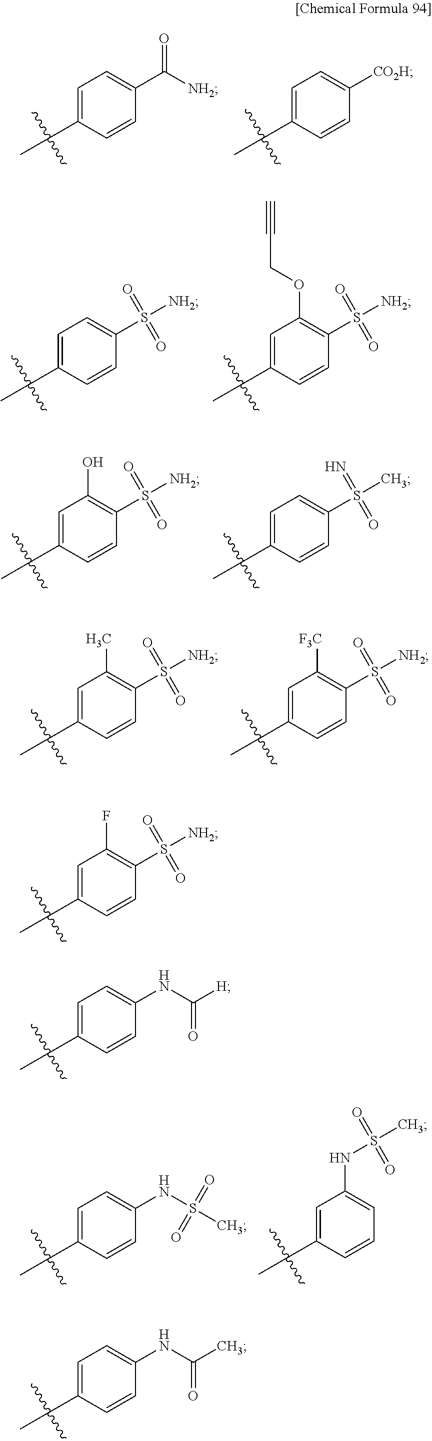

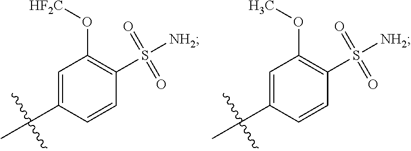

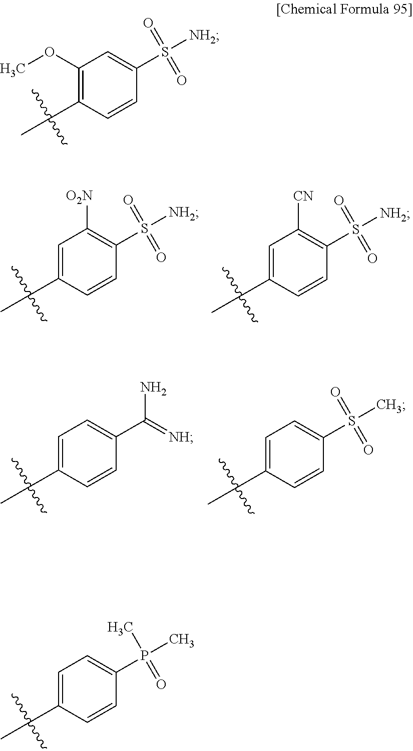 [SMILES: *c1ccc(S(N)(=O)=O)cc1OC ; *c1ccc(S(N)(=O)=O)c([N+](=O)[O-])c1 ; *c1ccc(S(N)(=O)=O)c(C#N)c1 ; *c1ccc(C(=N)N)cc1 ; *c1ccc(S(C)(=O)=O)cc1 ; *c1ccc(P(C)(C)=O)cc1]

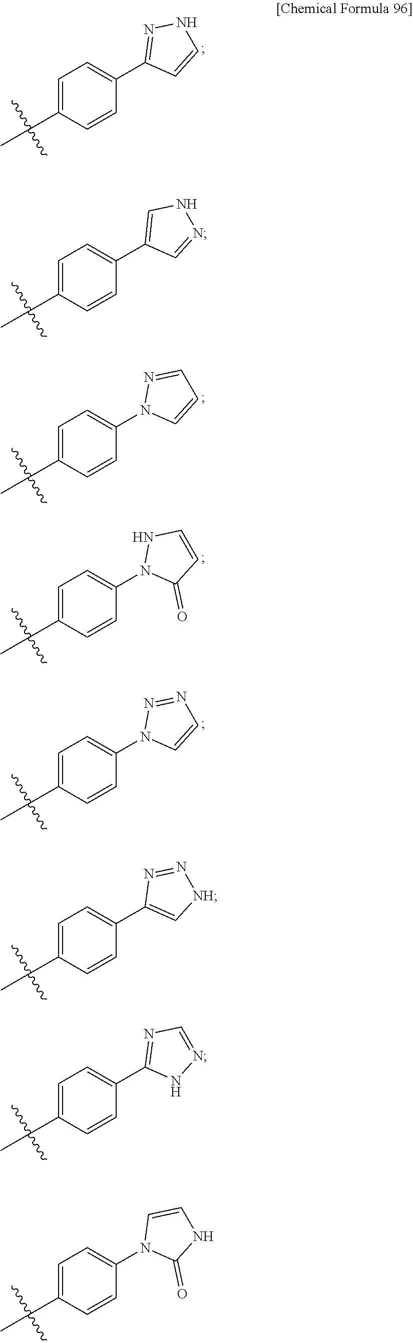

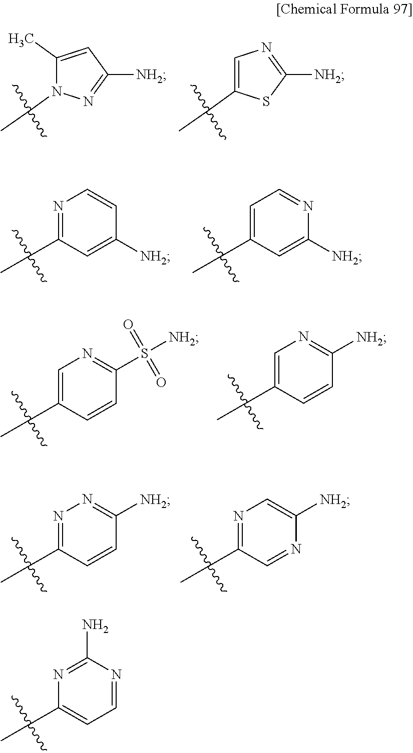

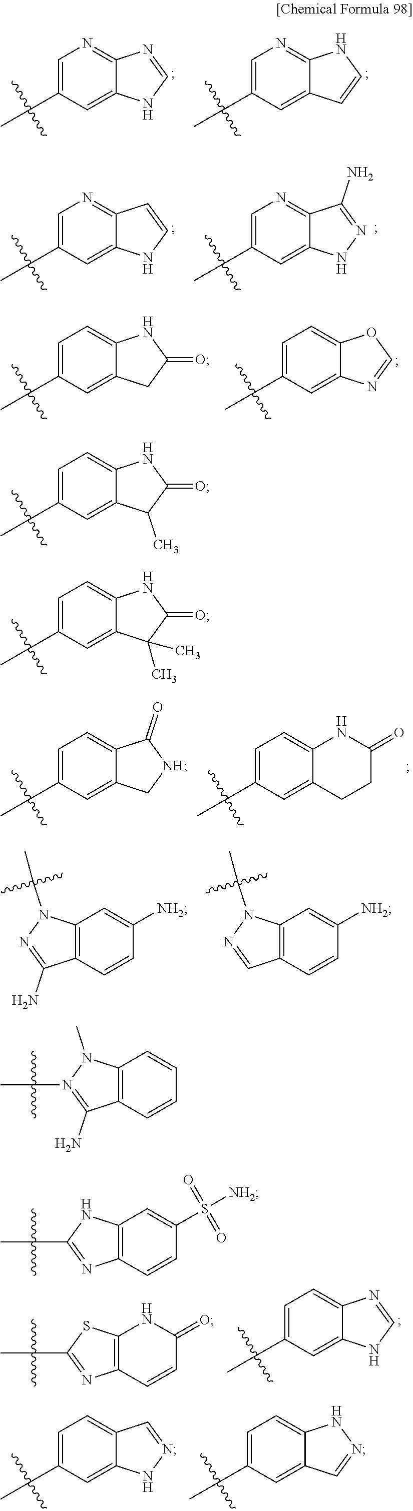

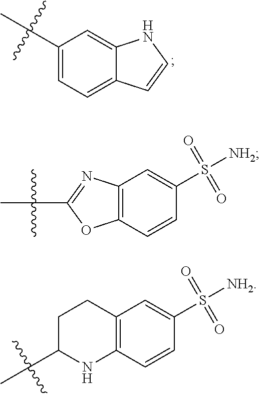 [SMILES: *c1ccc2cc[nH]c2c1 ; *c1nc2cc(S(N)(=O)=O)ccc2o1 ; *C1CCc2cc(S(N)(=O)=O)ccc2N1]

(2-5) R² is a hydrogen atom, a deuterium atom, C₁₋₆ alkyl, C₁₋₆ alkenyl, or C₁₋₆ alkynyl;
(2-5-1) R² is a hydrogen atom.

In formula (2), variables can be the following.

TABLE 44

| A | X | L | R¹ | R² |
|---|---|---|---|---|
| 2-1-1 | 2-2-1 | 2-3-1 | 2-4-1 | 2-5-1 |
| 2-1-2 | 2-2-1 | 2-3-1 | 2-4-1 | 2-5-1 |
| 2-1-1 | 2-2-1 | 2-3-1 | 2-4-2 | 2-5-1 |
| 2-1-2 | 2-2-1 | 2-3-1 | 2-4-2 | 2-5-1 |

While the preferred variables in the compound of the present disclosure represented by formula (3) are described below, the technical scope of the present disclosure is not limited to the scope of the compounds described below.

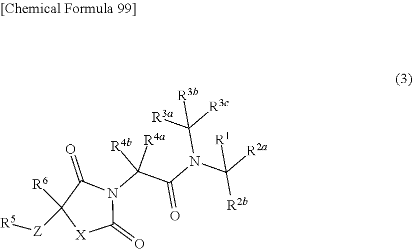

(3-1)
   X is —NH— or O—;
(3-1-1)
   X is —NH—;
(3-1-2)
   X is —O—.
(3-2)
   Z is a direct bond or —C(R^{7a})(R^{7b})—;
(3-2-1)
   Z is a direct bond.
(3-2-2)
   Z is —C(R^{7a})(R^{7b})—.
(3-3)
   R¹ is carbocyclyl or heterocyclyl;
(3-3-1)
   R¹ is carbocyclyl;
(3-3-2)
   R¹ is phenyl;
(3-4)
   R^{2a} and R^{2b} are each independently a hydrogen atom, a deuterium atom, C₁₋₆ alkyl, C₁₋₆ alkenyl, or C₁₋₆ alkynyl;
(3-4-1)
   R^{2a} and R^{2b} are each a hydrogen atom;
(3-5)
   R^{3a} is carbocyclyl or heterocyclyl, and R^{3b} is C₁₋₆ alkyl, C₁₋₆ alkenyl, C₁₋₆ alkynyl, or carbocyclyl, or R^{3a} and R^{3b} are each independently C₁₋₆ alkyl, C₁₋₆ alkenyl, or C₁₋₆ alkynyl, wherein R^{3a} and R^{3b}, together with the carbon atom to which they are attached, may form carbocyclyl or heterocyclyl;
(3-5-1)
   R^{3a} is carbocyclyl, and R^{3b} is C₁₋₆ alkyl.
(3-5-2)
   R^{3a} is cycloalkyl, and R^{3b} is C₁₋₆ alkyl.
(3-5-3)
   R^{3a} is cyclopropyl, and R^{3b} is methyl.
(3-6)
   R^{3c} is a hydrogen atom or a deuterium atom;
(3-6-1)
   R^{3c} is a hydrogen atom.
(3-7)
   R^{4a} and R^{4b} are each independently a hydrogen atom, a deuterium atom, C₁₋₆ alkyl, C₁₋₆ alkenyl, or C₁₋₆ alkynyl;
(3-7-1)
   R^{4a} and R^{4b} are each a hydrogen atom.
(3-8)
   R⁵ is carbocyclyl or heterocyclyl;
(3-8-1)
   R⁵ is aryl or heteroaryl;
(3-8-2)
   R⁵ has one of the following structures:

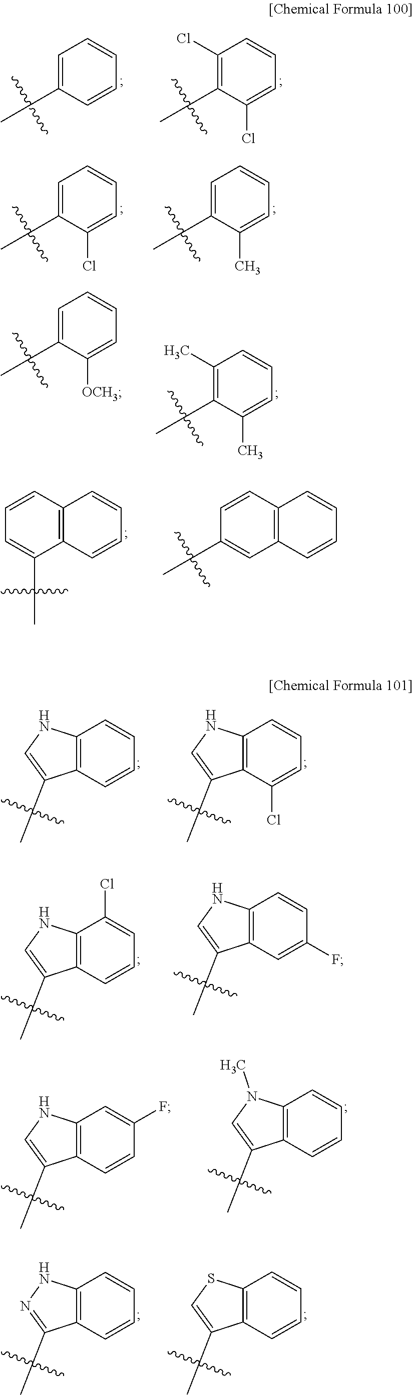

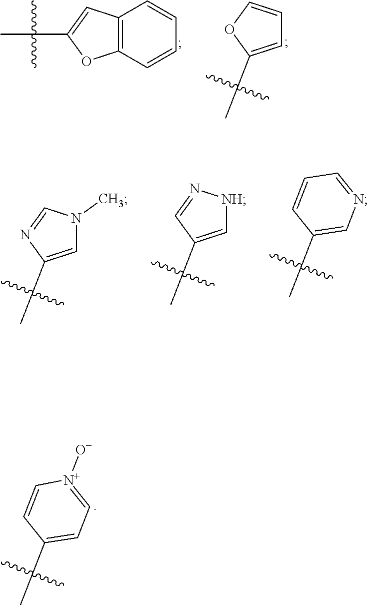 [SMILES: *c1cc2ccccc2o1 ; *c1ccco1 ; *c1cn(C)cn1 ; *c1cn[nH]c1 ; *c1cccnc1 ; *c1cc[n+]([O-])cc1]

(3-9)
   R⁶ is, when Z is a direct bond, a hydrogen atom or a deuterium atom, or is, when Z is —C(R^{7a})(R^{7b})—, a hydrogen atom, a deuterium atom, C₁₋₆ alkyl, C₁₋₆ alkenyl, or C₁₋₆ alkynyl;
(3-9-1)
   R⁶ is a hydrogen atom.
(3-10)
   R^{7a} and R^{7b} are each independently a hydrogen atom, a deuterium atom, C₁₋₆ alkyl, C₁₋₆ alkenyl, or C₁₋₆ alkynyl.

In formula (3), variables can be the following.

TABLE 45

| | | | [symbol] and | [symbol] and | | [symbol] and | | |
|---|---|---|---|---|---|---|---|---|
| X | Z | R¹ | [symbol] | [symbol] | R [symbol] | [symbol] | R [symbol] | R [symbol] |
| 3-1-1 | 3-2-1 | 3-3-2 | 3-4-1 | 3-5-1 | 3-6-1 | 3-7-1 | 3-8-2 | 3-9-1 |
| 3-1-2 | 3-2-1 | 3-3-2 | 3-4-1 | 3-5-1 | 3-6-1 | 3-7-1 | 3-8-2 | 3-9-1 |
| 3-1-1 | 3-2-2 | 3-3-2 | 3-4-1 | 3-5-1 | 3-6-1 | 3-7-1 | 3-8-2 | 3-9-1 |
| 3-1-2 | 3-2-2 | 3-3-2 | 3-4-1 | 3-5-1 | 3-6-1 | 3-7-1 | 3-8-2 | 3-9-1 |

[symbol]

indicates data missing or illegible when filed

While the preferred variables in the compound of the present disclosure represented by formula (4) are described below, the technical scope of the present disclosure is not limited to the scope of the compounds described below.

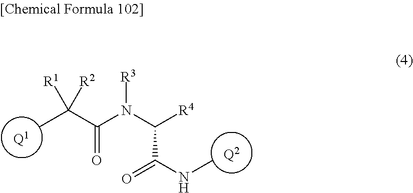

(4-1)
   ring Q³ represents a phenyl group optionally having 1 to 3 substituents independently selected from group A described below, or a 5- or 6-membered aromatic heterocyclic group having 1 to 3 nitrogen atoms having 1 to 3 substituents independently selected from group A described below within a ring,
   wherein group A is a halogen atom, a hydroxy group, a carboxy group, a C₁₋₆ alkyl group, a hydroxy C₁₋₆ alkyl group, a C₁₋₆ alkoxy group, a halogeno C₁₋₆ alkoxy group, a C₁₋₆ alkoxycarbonyl group, a C₂₋₇ alkanoyl group, a halogeno C₂₋₇ alkanoyl group, a C₂₋₁₀ alkanoylamino group, a C₁₋₆ alkylsulfonyl group, a C₁₋₆ alkylsulfonylamino group, a C₃₋₇ cycloalkylsulfonylamino group, a phenyl group, a phenylsulfonylamino group, a carbamoyl group, a C₁₋₆ alkylcarbamoyl group, a di-C₁₋₆ alkylcarbamoyl group, a benzyloxycarbonyl group, a C₃₋₇ cycloalkylsulfonylcarbamoyl group, a halogeno C₁₋₆ alkylsulfonyloxy group, and a phenylsulfonyl group;
(4-1-1)
   ring Q¹ is a p-hydroxyphenyl group, a p-methoxyphenyl group, a p-deuterated methyloxyphenyl group, a p-fluoromethoxyphenyl group, a p-difluoromethoxyphenyl group, a p-acetylphenyl group, a p-trifluoromethoxyphenyl group, a p-trifluoromethylmethoxyphenyl group, a p-trifluoroacetylphenyl group, a p-(2-hydroxypropan-2-yl)phenyl group, a 6-methoxy-3-pyridinyl group, an m-fluoro-p-methoxyphenyl group, or an m-fluoro-p-difluoromethoxyphenyl group.
(4-2)
   ring Q² represents a phenyl group optionally having 1 to 3 substituents independently selected from group B described below, a naphthyl group optionally having 1 to 3 substituents independently selected from group B described below, a 5- or 6-membered aromatic heterocyclic group having 1 to 3 nitrogen atoms optionally having 1 to 3 substituents independently selected from group B described below within a ring, or an 8- to 10-membered bicyclic aromatic heterocyclic group optionally having 1 to 4 heteroatoms independently selected from the group consisting of a nitrogen atom, an oxygen atom, and a sulfur atom optionally having 1 to 3 substituents independently selected from group B described below within a ring,
   wherein group B is a halogen atom, a cyano group, an amino group, a C₁₋₆ alkyl group, a C₁₋₆ alkoxy group, a hydroxy C₁₋₆ alkyl group, a C₁₋₆ alkylamino group, a C₁₋₆ alkylamino C₁₋₆ alkyl group, a morpholinyl C₁₋₆ alkyloxy group, a phenyl group, a benzyloxy group, a C₁₋₆ alkoxy C₁₋₆ alkyl group, a hydroxy group, a halogeno C₁₋₆ alkyl group, a C₁₋₆ alkoxycarbonyl group, a C₂₋₇ alkanoylamino group, a halogeno C₁₋₆ alkoxy group, a C₁₋₆ alkoxy C₁₋₆ alkoxy group, a C₁₋₆ alkylsulfonylamino group, a morpholinyl C₁₋₆ alkyl group, and a C₁₋₆ alkylsulfonyl group;
(4-2-1)
   ring Q² is

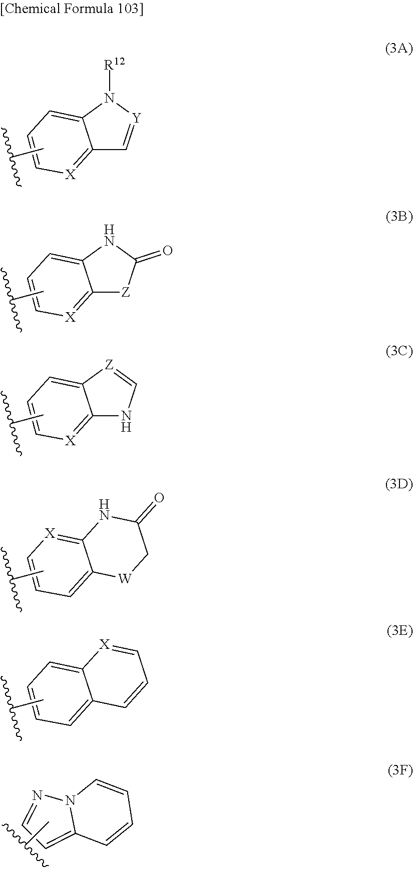

wherein
   X represents a nitrogen atom or —CR¹³,
   Y represents a nitrogen atom or —CR¹⁴,
   Z represents —NH or —CH in formula (3B), and a nitrogen atom or —CH in formula (3C),
   W represents an oxygen atom or —CH₂,
   R¹² represents a hydrogen atom, a fluorine atom, or a cyano group,
   R¹³ represents a hydrogen atom, a fluorine atom, or a cyano group, and
   R¹⁴ represents a hydrogen atom, a C₁₋₆ alkyl group, a hydroxy C₁₋₆ alkyl group, a C₁₋₆ alkylamino C₁₋₆ alkyl group, or a phenyl group.
(4-3)
   R¹ and R² each independently represents a C₁₋₆ alkyl group or a C₁₋₆ alkoxy group, or
   R¹ and R², together with the carbon atom to which R¹ and R² are attached, are a 3- to 7-membered cycloalkyl ring optionally having 1 to 3 substituents independently selected from group C described below, a tetrahydropyran ring optionally having 1 to 3 substituents independently selected from group C described below, or a dioxane ring optionally having 1 to 3 substituents independently selected from group C described below, and
   group C is a halogen atom, a C₁₋₆ alkyl group, and a C₁₋₆ alkoxy group;
(4-3-1)
   R¹ and R² each independently represents a methyl group, or
   R¹ and R², together with the carbon atom to which R¹ and R² are attached, form a 3,3-difluorocyclobutane ring, a 3,3-dimethylcyclobutane ring, a cyclopentane ring, a cyclohexane ring, a 4,4-difluorocyclohexane ring, or a 4-tetrahydropyran ring.
(4-4)
   R³ represents a hydrogen atom, a C₁₋₆ alkyl group, or a hydroxy C₁₋₆ alkyl group,
   R⁴ represents a hydrogen atom, a C₁₋₆ alkyl group, a hydroxy C₁₋₆ alkyl group, or a C₁₋₆ alkylsulfonyl C₁₋₆ alkyl group, or
   R³ and R⁴, together with the nitrogen atom to which R³ is attached and the carbon atom to which R⁴ is attached, may form an azetidine ring optionally having 1 to 3 substituents independently selected from group D described below, a pyrrolidine ring optionally having 1 to 3 substituents independently selected from group D described below, a hexamethyleneimine ring optionally having 1 to 3 substituents independently selected from group D described below, a thiazolidine ring optionally having 1 to 3 substituents independently selected from group D described below, a 1-oxothiazolidine ring optionally having 1 to 3 substituents independently selected from group D described below, a 1,1′-dioxothiazolidine ring optionally having 1 to 3 substituents independently selected from group D described below, or a 4-oxopyrrolidine ring optionally having 1 to 3 substituents independently selected from group D described below;
   group D is a halogen atom, a hydroxy group, a C₁₋₆ alkyl group, a C₁₋₆ alkoxy group, a C₁₋₆ alkoxy C₁₋₆ alkoxy group, a C₂₋₆ alkynyl group, a C₂₋₇ alkanoylamino group, an amino group, and a di-C₁₋₆ alkylamino group,
or a prodrug thereof or a pharmaceutically acceptable salt thereof;
(4-4-1)
   R³ represents a methyl group or a deuterated methyl group, and R⁴ represents a hydroxymethyl group or a 1-hydroxyethyl group, or
   R³ and R⁴, together with the nitrogen atom to which R³ is attached and the carbon atom to which R⁴ is attached, represents,

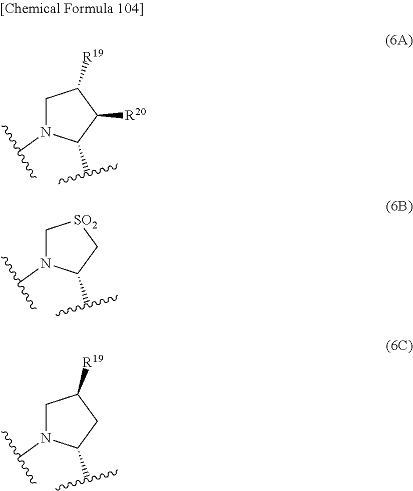

wherein
   R¹⁹ represents a hydrogen atom, a fluorine atom, or a hydroxy group, and
   R²⁰ represents a hydrogen atom or a hydroxy group.

In formula (4), variables can be 4-1-1, 4-2-1, 4-3-1, and 4-4-1.

While the preferred variables in the compound of the present disclosure represented by formula (5) are described below, the technical scope of the present disclosure is not limited to the scope of the compounds described below.

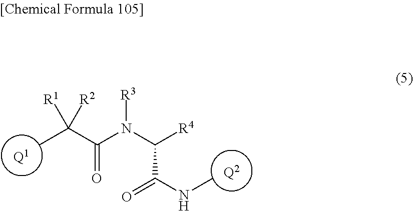

(5-1)
   ring Q¹ represents a 3- to 7-membered cycloalkyl group optionally having 1 to 3 substituents independently selected from group A described below, a 3- to 7-membered heterocycloalkyl group having 1 to 2 heteroatoms independently selected from the group consisting of a nitrogen atom, an oxygen atom, and a sulfur atom optionally having 1 to 3 substituents independently selected from group A described below within a ring, or an 8- to 10-membered bicyclic heterocycloalkyl group having 1 to 3 heteroatoms independently selected from the group consisting of a nitrogen atom, an oxygen atom, and a sulfur atom optionally having 1 to 3 substituents independently selected from group A described below within a ring,
   wherein group A is a halogen atom, a hydroxy group, a carboxy group, an amino group, a C₁₋₆ alkyl group, a halogeno C₁₋₆ alkyl group, a hydroxy C₁₋₆ alkyl group, a C₁₋₆ alkoxy C₁₋₆ alkyl group, a C₁₋₆ alkoxy group, a halogeno C₁₋₆ alkoxy group, a C₁₋₆ alkoxy C₁₋₆ alkoxy group, a C₂₋₇ alkanoyl group, a hydroxy C₂₋₇ alkanoyl group, a C₂₋₇ alkanoylamino group, a C₁₋₆ alkylsulfonyl group, a C₁₋₆ alkylsulfonylamino group, a benzyl group, a benzyloxy group, and an oxo group;
(5-1-1)
   ring Q¹ is

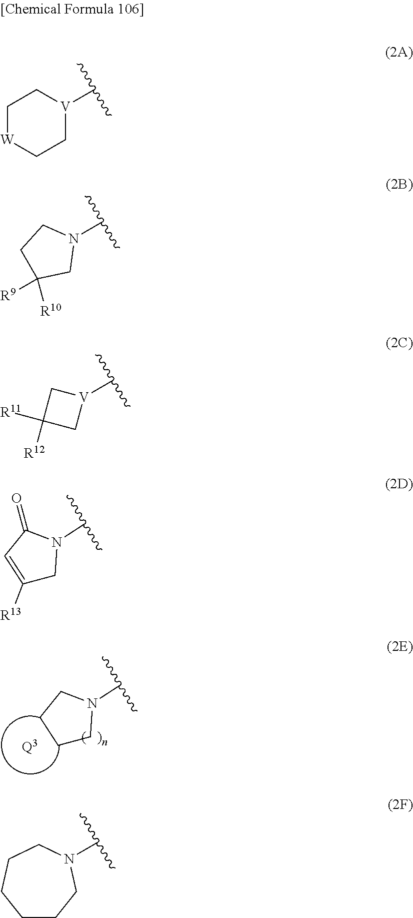

wherein
   V represents a nitrogen atom or —CR⁵,
   W represents an oxygen atom, —NR⁶, —CR⁷R⁸, or —SO₂,
   R⁵ represents a hydrogen atom or a hydroxy group,
   R⁶ represents a hydrogen atom, a C₁₋₆ alkyl group, a C₂₋₇ alkanoyl group, a hydroxy C₂₋₇ alkanoyl group, a C₁₋₆ alkylsulfonyl group, or a benzyl group,
   R⁷ and R⁸ each independently represents a hydrogen atom, a halogen atom, each independently a hydrogen atom, a halogen atom, a hydroxy group, a carboxy group, an amino group, a C₁₋₆ alkyl group, a halogeno C₁₋₆ alkyl group, a hydroxy C₁₋₆ alkyl group, an alkoxy C₁₋₆ alkyl group, a C₁₋₆ alkoxy group, a C₁₋₆ alkoxy C₁₋₆ alkoxy group, a halogeno C₁₋₆ alkoxy group, a C₂₋₇ alkanoylamino group, a C₁₋₆ alkylsulfonylamino group, or a benzyl group,
   R⁹ and R¹⁰ each independently represents a hydrogen atom, a halogen atom, or a C₁₋₆ alkoxy group,
   R¹¹ and R¹² each independently represents a hydrogen atom, a hydroxy group, a C₁₋₆ alkoxy group, or a benzyloxy group, or
   R¹¹ and R¹² together form an oxo group,
   R¹³ represents a C₁₋₆ alkoxy group,
   ring Q³ represents a benzene ring, a pyrazole ring, or a tetrahydrofuran ring, and
   n represents 1 or 2.
(5-1-2)
   ring Q¹ is

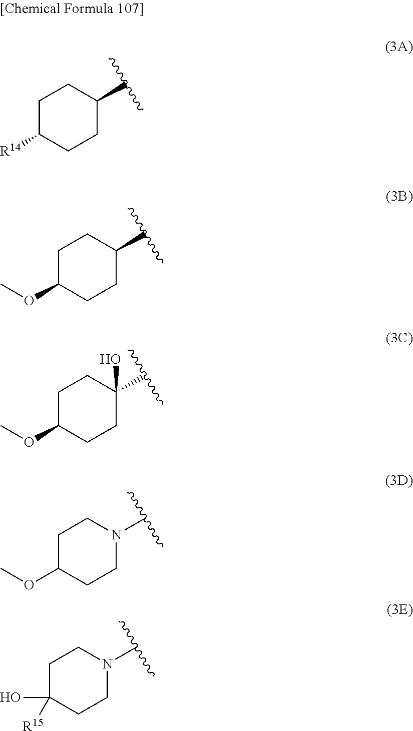

wherein
   R¹⁴ represents a methoxy group, a difluoromethoxy group, or a trifluoromethoxy group, and
   R¹⁵ represents a methyl group or a trifluoromethyl group.
(5-2)
   ring Q² represents a phenyl group optionally having 1 to 3 substituents independently selected from group B described below, a naphthyl group optionally having 1 to 3 substituents independently selected from group B described below, a 5- or 6-membered aromatic heterocyclic group having 1 to 3 nitrogen atoms optionally having 1 to 3 substituents independently selected from group B described below within a ring, or an 8- to 10-membered bicyclic aromatic heterocyclic group having 1 to 4 heteroatoms independently selected from the group consisting of a nitrogen atom, an oxygen atom, and a sulfur atom optionally having 1 to 3 substituents independently selected from group B described below within a ring,
   group B is a halogen atom, a cyano group, an amino group, a C₁₋₆ alkyl group, a C₁₋₆ alkoxy group, a hydroxy C₁₋₆ alkyl group, a C₁₋₆ alkylamino group, a C₁₋₆ alkylamino C₁₋₆ alkyl group, a morpholinyl C₁₋₆ alkyloxy group, a phenyl group, a benzyloxy group, a C₁₋₆ alkoxy C₁₋₆ alkyl group, a hydroxy group, a halogeno C₁₋₆ alkyl group, a C₁₋₆ alkoxycarbonyl group, a C₂₋₇ alkanoylamino group, a halogeno C₁₋₆ alkoxy group, a C₁₋₆ alkoxy C₁₋₆ alkoxy group, a C₁₋₆ alkylsulfonylamino group, a morpholinyl C₁₋₆ alkyl group, and a C₁₋₆ alkylsulfonyl group;
(5-2-1)
   ring Q² is

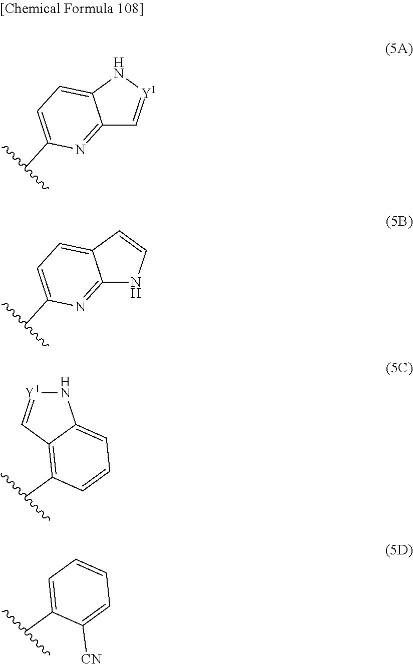

wherein
   Y¹ represents a nitrogen atom or —CH.
(5-3)
   R¹ and R² each independently represents a C₁₋₆ alkyl group or a C₁₋₆ alkoxy group, or
   R¹ and R², together with the carbon atom to which R¹ and R² are attached, represent a 3- to 7-membered cycloalkyl ring optionally having 1 to 3 substituents independently selected from group C described below, a tetrahydropyran ring optionally having 1 to 3 substituents independently selected from group C described below, or a dioxane ring optionally having 1 to 3 substituents independently selected from group C described below, and
   group C is a halogen atom, a C₁₋₆ alkyl group, and a C₁₋₆ alkoxy group;
(5-3-1)
   R¹ and R² are each a methyl group, or
   R¹ and R², together with the carbon atom to which R¹ and R² are attached, form a cyclopentane ring, a cyclohexane ring, or a 4,4-difluorocyclohexane ring.
(5-4)
   R³ represents a hydrogen atom, a C₁₋₆ alkyl group, or a hydroxy C₂₋₆ alkyl group,
   R⁴ represents a hydrogen atom, a C₁₋₆ alkyl group, a hydroxy C₁₋₆ alkyl group, or a C₁₋₆ alkylsulfonyl C₁₋₆ alkyl group, or
   R³ and R⁴, together with the nitrogen atom to which R³ is attached and the carbon atom to which R⁴ is attached, may form an azetidine ring optionally having 1 to 3 substituents independently selected from group D described below, a pyrrolidine ring optionally having 1 to 3 substituents independently selected from group D described below, a hexamethyleneimine ring optionally having 1 to 3 substituents independently selected from group D described below, a thiazolidine ring optionally having 1 to 3 substituents independently selected from group D described below, a 1-oxothiazolidine ring optionally having 1 to 3 substituents independently selected from group D described below, a 1,1-dioxothiazolidine ring optionally having 1 to 3 substituents independently selected from group D described below, or a 4-oxopyrrolidine ring optionally having 1 to 3 substituents independently selected from group D described below,
   group D is a halogen atom, a hydroxy group, a C₁₋₆ alkyl group, a C₁₋₆ alkoxy group, a C₁₋₆ alkoxy C₁₋₆ alkoxy group, a C₂₋₆ alkynyl group, a C₂₋₇ alkanoylamino group, an amino group, and a di-C₁₋₆ alkylamino group;
(5-4-1)
   R³ is a methyl group, and R⁴ is a methyl group or a hydroxymethyl group, or
   R³ and R⁴, together with the nitrogen atom to which R³ is attached and the carbon atom to which R³ is attached, are

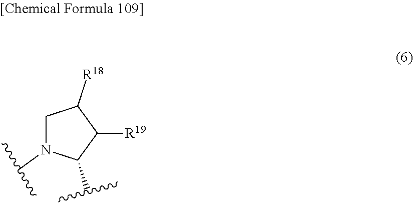

wherein
   R¹⁸ represents a hydrogen atom, a halogen atom, a hydroxy group, a C₁₋₆ alkoxy group, or a C₁₋₆ alkoxy C₁₋₆ alkoxy group, and
   R¹⁹ represents a hydrogen atom or a hydroxy group.

In formula (5), variables can be the following.

TABLE 46

| Q¹ | Q² | R¹ and R² | R³ |
|---|---|---|---|
| 5-1-1 | 5-2-1 | 5-3-1 | 5-4-1 |
| 5-2-1 | 5-2-1 | 5-3-1 | 5-4-1 |

While the preferred variables in the compound of the present disclosure represented by formula (6) are described below, the technical scope of the present disclosure is not limited to the scope of the compounds described below.

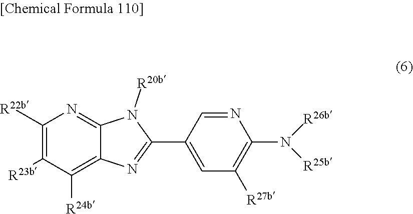

(6-1)
   R^{20b′} is C₁₋₂ alkyl (wherein the alkyl group is substituted with phenyl substituted with pyrimidinyl, pyrazolyl, pyrazolyl substituted with C₁₋₃ alkyl, pyrazinyl, pyrazinyl substituted with C₁₋₃ alkyl, piperazinyl, piperazinyl substituted with oxo, piperazinyl substituted with C₁₋₃ alkyl, oxazolyl, oxazolyl substituted with C₁₋₃ alkyl, imidazolyl, imidazolyl substituted with C₁₋₃ alkyl, morpholinyl, morpholinyl substituted with 1 to 2 C₁₋₃ alkyl, morpholinyl substituted with oxo, dioxanyl, dioxanyl substituted with C₁₋₃ alkyl, 4,5,6,7-tetrahydropyrazolo[1,5-a]pyrazine, triazolyl, triazolyl substituted with C₁₋₃ alkyl, thiazolyl, thiazolyl substituted with C₁₋₃ alkyl, cyclopentyloxy, C₁₋₆ alkoxy, C₁₋₆ alkoxy substituted with 1 to 6 fluoro, C₁₋₆ alkoxy substituted with hydroxy, tetrahydrofuran, pyridyl, pyridyl substituted with bromo, or pyridyl substituted with pyrimidinyl);
(6-1-1)
   R^{20b′} is C₁₋₂ alkyl (wherein the alkyl group is substituted with pyrazolyl substituted with C₁₋₃ alkyl or C₁₋₆ alkoxy).
(6-2)
   R^{22b′}, R^{23b′}, and R^{24b′} are each independently selected from a hydrogen atom, fluoro, chloro, bromo, —OH, boronic acid, 1,3,6,2-dioxazaborocane-4,8-dione, —CN, —C(O)NHCH₃, —C(O)NHCH₂CH₃, —C(O)NHCH₂CF₂H, —C(O)NHCH₂CH₂OH, —C(O)NHCH₂CH₂SO₂CH₃, —C(O)NHOCH₃, —C(O)NH₂, —C(O)OCH₃, —C(O)NHCH₂ cyclopropyl, —C(O)NH cyclobutyl (wherein the group is optionally substituted with hydroxy), —CH₂ morpholinyl, —CH₂OH, —CH₂NHCH₂CF₃, —CH₂NHCH₂CH₂SO₂CH₃, —CH₂SO₂CH₃, —CH(OH)CF₃, —CH₃, —CF₃, —OCH₃, —OCD₃, —NHC(O)CH₃, —NH₂, —NHSO₂CH₃, morpholinyl, pyrazolyl, oxazolyl, or oxazolyl substituted with 1 to 2 methyl;
   R^{23b′} and R^{21b′}, together with the carbon atom to which they are attached, may form oxaborolyl (wherein the group is optionally substituted with hydroxy);
(6-2-1)
   R^{22b′} is a hydrogen atom, R^{23b′} is —C(O)NHCH₃, boronic acid, or —C(O)NH₂, and R^{24b′} is a hydrogen atom or —CH₃.
(6-3)
   R^{25b′} and R^{26b′} are each independently selected from C₁₋₃ alkyl, C₁₋₃ alkyl substituted with 1 to 3 fluoro, or cyclopropyl;
   wherein R^{25b′} and R^{26b′}, together with the nitrogen atom to which they are attached, may form azetidinyl or pyrrolidinyl (wherein the group is optionally substituted with 1 to 2 C₁₋₃ alkyl, or C₁₋₃ alkyl substituted with 1 to 3 fluoro), or
   one of R^{25b′} and R^{26b′} may form pyrrolidinyl or morpholinyl with R^{27b′} and any one heteroatom (wherein the group is optionally substituted with 1 to 4 C₁₋₃ alkyl);
(6-3-1)
   R^{25b′} and R^{26b′} are each independently C₁₋₃ alkyl, or R^{25b′} and R^{26b′}, together with the nitrogen atom to which they are attached, form pyrrolidinyl (wherein the group is optionally substituted with 1 to 2 C₁₋₃ alkyl).
(6-4)
   R^{27b′} is selected from a hydrogen atom and fluoro;
   wherein one of R^{25b′} and R^{26b′} may form pyrrolidinyl or morpholinyl with R^{27b′} and any one heteroatom (wherein the group is optionally substituted with 1 to 4 C₁₋₃ alkyl);
(6-4-1)
   R^{27b′} is a hydrogen atom.

In formula (6), variables can be 6-1-1, 6-2-1, 6-3-1, and 6-4-1.

While the preferred variables in the compound of the present disclosure represented by formula (7) are described below, the technical scope of the present disclosure is not limited to the scope of the compounds described below.

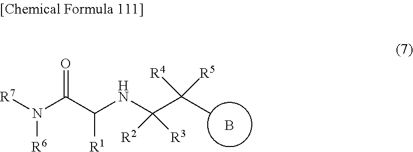

(7-1)
   ring B is aryl, heterocyclyl, or heteroaryl (wherein the ring is each optionally substituted with 1 to 4 substituents selected from R^{b});
(7-1-1)
   ring B is phenyl optionally substituted with 1 to 3 substituents selected from R^{b}.
(7-2)
   R⁶ is a hydrogen atom or C₁₋₆ alkyl;
   R⁷ is aryl or heteroaryl (wherein the group is each substituted with a substituent selected from R^{f}, and optionally substituted with 1 to 4 substituents selected from R^{a});
   wherein R⁶ and R⁷, together with the nitrogen ring to which they are attached, may form a fused bicyclic heterocyclyl optionally substituted with 1 to 4 groups selected from R^{a};
(7-2-1)
   R⁶ is a hydrogen atom;
   R⁷ is selected from phenyl, 2-pyridinyl, 3-pyridinyl, 3-pyridinyl, pyrimidin-5-yl, and quinolin-6-, and the group is each substituted with a substituent selected from R^{f}, and optionally substituted with 1 to 4 substituents selected from R^{a},
(7-3)
   R¹ is C₁₋₆ alkyl, C₁₋₆ haloalkyl, C₂₋₆ alkenyl, —C₁₋₆ alkyl OR^{c}, —C₁₋₆ alkyl N(R^{d})₂, —C₁₋₆ alkyl C(O)OR^{d}, —C₁₋₆ alkyl OC₁₋₆ alkyl N(R^{d})₂, —C₁₋₆ alkyl SOR^{d}, —C₁₋₆ alkyl S(O)₂R^{d}, —C₁₋₆ alkyl SON(R^{d})₂, —C₁₋₆ alkyl SO₂N(R^{d})₂, —C₁₋₆ alkylcycloalkyl, —C₁₋₆ alkylheterocyclyl, —C₁₋₆ alkylheteroaryl, —C₁₋₆ alkylaryl, cycloalkyl, aryl, heteroaryl, or heterocyclyl (wherein each of the cycloalkyl, heterocyclyl, aryl, and heteroaryl, alone or attached to —C₁₋₆ alkylcycloalkyl, —C₁₋₆ alkylheterocyclyl, —C₁₋₆ alkylheteroaryl, or —C₁₋₆ alkylaryl, is optionally substituted with 1 to 3 groups selected from R^{c});
(7-3-1)
   R¹ is phenyl or 4-pyrazolyl optionally substituted with 1 to 3 groups selected from R^{c};
(7-4)
   R¹, R³, R⁴, and R⁵ are each independently a hydrogen atom or C₁₋₆ alkyl (wherein the C₁₋₆ alkyl is optionally substituted with 1 to 2 substituents selected from a halogen atom, —C(O)OR^{d}, —OC₁₋₆ alkyl N(R^{d})₂, —C₁₋₆ alkyl N(R^{d})₂, —N(R^{d})₂, —NR^{d}C₁₋₆ alkyl OR^{d}, —SOR^{d}, —S(O)₂R^{d}, —SON(R^{d})₂, —SO₂N(R^{d})₂, C₃₋₁₀ cycloalkyl, C₅₋₁₀ heterocyclyl, C₅₋₁₀ heteroaryl, and C₆₋₁₀ aryl);
(7-4-1)
   R² is hydrogen or methyl, R³ is hydrogen, R⁴ is hydrogen or methyl, and R⁵ is hydrogen,
(7-5)
   R^{a}, R^{b}, and R^{c} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O)OR^{d}, —C(O)N(R^{d})₂, —C(O)NR^{d}C₁₋₆ alkyl OR^{d}, —OC₁₋₆ alkyl N(R^{d})₂, —C₁₋₆ alkyl C(O)N(R^{d})₂, —C₁₋₆ alkyl N(R^{d})₂, —N(R^{d})₂, —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)₂R^{d}, —SON(R^{d})₂, —SO₂N(R^{d})₂, —SF₅, —O— cycloalkyl, —O—C₁₋₄ alkyl-aryl, —C₁₋₆ alkylcycloalkyl, —C₁₋₆ alkylaryl, —C₁₋₆ alkylheteroaryl, —C₁₋₆ alkylheterocyclyl, cycloalkyl, heterocyclyl, heteroaryl, or aryl (wherein each of the cycloalkyl, heterocyclyl, aryl, and heteroaryl, alone or attached to —O-cycloalkyl, —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 groups selected from halogen, C₁₋₆ alkyl, C₁₋₆ haloalkyl, C₁₋₆ alkoxy, C₁₋₆ haloalkoxy, —N(R^{d})₂, —C(O)R^{d}, and —C₁₋₆ alkyl OR^{d});
(7-5-1)
   R^{a} is methyl or halo,
   R^{b} is halo, cyano, or —SO₂NH₂, and
   R^{c} is C₁₋₆ alkyl, halo, or CN.
(7-6)
   R^{d} is independently a hydrogen atom, C₁₋₆ haloalkyl, or C₁₋₆ alkyl;
(7-6-1)
   R^{d} is a hydrogen atom or C₁₋₃ alkyl.
(7-7)
   R^{f} is independently cycloalkyl, heterocyclyl, heteroaryl, or aryl (wherein each of the cycloalkyl, heterocyclyl, aryl, and heteroaryl is optionally substituted with 1 to 3 substituents selected from a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O)OR^{d}, —C(O)N(R^{d})₂, —C(O)NR^{d}C₁₋₆ alkyl OR^{d}, —OC₁₋₆ alkyl N(R^{d})₂, —C₁₋₆ alkyl C(O)N(R^{d})₂, —C₁₋₆ alkyl N(R^{d})₂, —N(R^{d})₂, —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)₂R^{d}, —SON(R^{d})₂, —SO₂N(R^{d})₂, —SF₅, and —O-cycloalkyl);
(7-7-1)
   R^{f} is optionally substituted with 1 to 3 substituents selected from pyrazolyl, pyridazinyl, azetidinyl, piperazinyl, piperidinyl, triazolyl, pyridinyl, and pyrrolidinyl, and each of said groups is optionally substituted with 1 to 3 substituents selected from methyl, difluoromethyl, methoxy, oxo, methylsulfonyl, acetyl, and dimethylaminocarbonylmethyl.

In formula (7), variables can be 7-1-1, 7-2-1, 7-3-1, 7-4-1, 7-5-1, 6-6-1, and 7-7-1.

While the preferred variables in the compound of the present disclosure represented by formula (8) are described below, the technical scope of the present disclosure is not limited to the scope of the compounds described below.

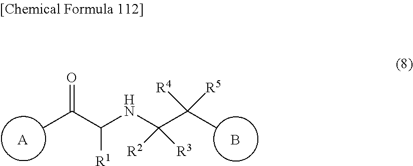

(8-1)
   ring A is bicyclic heteroaryl optionally substituted with 1 to 4 substituents selected from R^{a};
(8-1-1)
   ring A is selected from

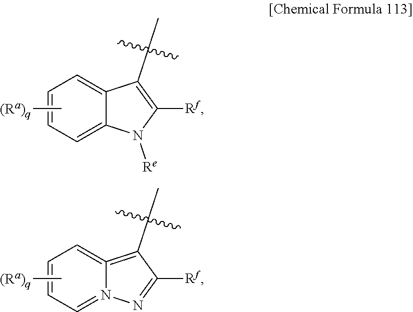

wherein q is 0, 1, or 2, R^{e} hydrogen, and R^{f} hydrogen.
(8-2)
   ring B is aryl, heterocyclyl, or heteroaryl optionally substituted with 1 to 4 substituents selected from R^{b};
(8-2-1)
   ring B is phenyl optionally substituted with 1 to 3 substituents selected from R^{b};
(8-3)
   R¹ is C₁₋₆ alkyl, C₁₋₆ haloalkyl, C₂₋₆ alkenyl, —C₁₋₆ alkyl OR^{c}, —C₁₋₆ alkyl N(R^{d})₂, —C₁₋₆ alkyl C(O)OR^{d}, —C₁₋₆ alkyl OC₁₋₆ alkyl N(R^{d})₂, —C₁₋₆ alkyl SOR^{d}, —C₁₋₆ alkyl S(O)₂R^{d}, —C₁₋₆ alkyl SON(R^{d})₂, —C₁₋₆ alkyl SO₂N(R^{d})₂, —C₁₋₆ alkylcycloalkyl, —C₁₋₆ alkylheterocyclyl, —C₁₋₆ alkylheteroaryl, —C₁₋₆ alkylaryl, cycloalkyl, aryl, heteroaryl, or heterocyclyl (wherein each of the cycloalkyl, heterocyclyl, aryl, and heteroaryl, alone or for —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 groups selected from R^{c});
(8-3-1)
   R¹ is phenyl.
(8-4)
   R², R³, R⁴, and R⁵ are each independently a hydrogen atom or C₁₋₆ alkyl (wherein the C₁₋₆ alkyl is optionally substituted with 1 to 2 substituents selected from a halogen atom, —C(O)OR^{d}, —OC₁₋₆ alkyl N(R^{d})₂, —C₁₋₆ alkyl N(R^{d})₂, —N(R^{d})₂, —NR^{d}C₁₋₆ alkyl OR^{d}, —SOR^{d}, —S(O)₂R^{d}, —SON(R^{d})₂, —SO₂N(R^{d})₂, cycloalkyl, heterocyclyl, heteroaryl, and aryl);
(8-4-1)
   R² is hydrogen or methyl, R³ is hydrogen, R⁴ is hydrogen or methyl, and R⁵ is hydrogen.
(8-5)
   R^{a}, R^{b}, and R^{c} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O)OR^{d}, —C(O)N(R^{d})₂, —C(O)NR^{d}C₁₋₆ alkyl OR^{d}, —OC₁₋₆ alkyl N(R^{d})₂, C₁₋₆ alkyl C(O)N(R^{d})₂, —C₁₋₆ alkyl N(R^{d})₂, —N(R^{d})₂, —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)R^{d}, —SON(R^{d})₂, —SO₂N(R^{d})₂, —SF₅, —O— cycloalkyl, —O-heterocyclyl, —O—C₁₋₄ alkyl-aryl, —C₁₋₆ alkylcycloalkyl, —C₁₋₆ alkylaryl, —C₁₋₆ alkylheteroaryl, —C₁₋₆ alkylheterocyclyl, cycloalkyl, heterocyclyl, heteroaryl, or aryl (wherein each of the cycloalkyl, heterocyclyl, aryl, and heteroaryl, alone or attached to —O-cycloalkyl, —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 of a halogen atom, oxo, C₁₋₆ alkyl, C₁₋₆ haloalkyl, C₁₋₆ alkoxy, C₁₋₆ haloalkoxy, —N(R^{d})₂, —C(O)R^{d}, and —C₁₋₆ alkyl OR^{d});
(8-5-1)
   R^{a} is —(CH₂)C(O)N(M^{e})₂, —C(O)NHCH₂CH₃, 1-methyl-4-pyrazolyl, 1-methyl-4-piperidyloxy, —C(O)NHCH₂CH₂N(M^{e})₂, —C(O)NHCH₂CH₂OCH₃, or —C(O)NHCH₂CH₂-(2-oxo-1-pyrrolidyl), and
   R^{b} is —CN or C₁.
(8-6)
   R^{d} are each independently a hydrogen atom, heterocyclyl, C₁₋₆ haloalkyl, or C₁₋₆ alkyl, wherein the heterocyclyl is optionally substituted with 1 to 2 substituents selected from C₁₋₄ haloalkyl and C₁₋₄ alkyl, and the C₁₋₆ alkyl is optionally substituted with SO₂C₁₋₄ alkyl or heterocyclyl (wherein the group is optionally substituted with oxo).

In formula (8), variables can be 8-1-1, 8-2-1, 8-3-1, 8-4-1, and 8-5-1.

While the preferred variables in the compound of the present disclosure represented by formula (9) are described below, the technical scope of the present disclosure is not limited to the scope of the compounds described below.

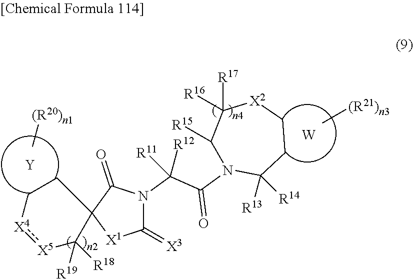

(9-1)
   X¹ is independently —O—, —NR¹—, or —S—;
(9-1-1)
   X¹ is —O— or —NH—;
(9-2)
   R¹ is independently a hydrogen atom, C₁₋₆ alkyl, or C₃₋₆ cycloalkyl;
(9-3)
   X² is independently —C(R²)(R³)—, —O—, —N(R⁴)—, or —S(O)ₙ₁—;
(9-3-1)
   X² is —C(R²)(R³)— or O—.
(9-4)
   R² and R³ are each independently a hydrogen atom, a deuterium atom, C₁₋₆ alkyl, C₁₋₆ haloalkyl, or C₃₋₆ cycloalkyl;
(9-4-1)
   R² and R³ are each a hydrogen atom.
(9-5)
   R⁴ is each independently a hydrogen atom, C₁₋₆ alkyl, C₃₋₆ cycloalkyl, —C(═O)(C₁₋₆ alkyl), —S(O)(C₁₋₆ alkyl), —C(═O)(C₃₋₆ cycloalkyl), or —S(O)₂ (C₃₋₆ cycloalkyl);
(9-6)
   X³ is each independently O or NH;
(9-6-1)
   X³ is O.
(9-7)

[symbol]

[Chemical Formula 115]
is a single bond or a double bond;
   wherein if

[symbol]

[Chemical Formula 116]
is a single bond, X⁴ is independently —C(R⁵)(R⁶)—, —O—, —C(═O)—, —NR⁷—, or —S(O)ₙ₁—;
   wherein if

[symbol]

[Chemical Formula 117]
is a single bond, X⁵ is independently —C(R⁸)(R⁹)—, —O—, —C(═O)—, —NR¹⁰—, —S(O)ₙ₁—, or a direct bond;
   wherein if

[symbol]

[Chemical Formula 118]
is a double bond, X⁴ is independently —C(R⁵)—;
   wherein if

[symbol]

[Chemical Formula 119]
is a double bond, X⁵ is independently —C(R⁸)—;
(9-7-1)

[symbol]

[Chemical Formula 120]
is a single bond, X⁴ is —C(R⁵)(R⁶)—, and X⁵ is a direct bond.
(9-8)
   R⁵ and R⁶ are each independently a hydrogen atom, OH, a halogen atom, CN, C₁₋₆ alkyl, C₁₋₆ haloalkyl, C₃₋₆ cycloalkyl, or C₁₋₆ alkoxy;
(9-8-1)
   R⁵ and R⁶ are each a hydrogen atom.
(9-9)
   R⁸ and R⁹ are each independently a hydrogen atom, OH, a halogen atom, or C₁₋₆ alkyl;
(9-10)
   R⁷ is each independently a hydrogen atom, C₁₋₆ alkyl, or C₃₋₆ cycloalkyl;
(9-11)
   R¹⁰ is each independently a hydrogen atom, C₁₋₆ alkyl, or C₃₋₆ cycloalkyl;
(9-12)
   Y is independently a C₆-10 aromatic ring or a C₅₋₁₀ heteroaromatic ring (wherein the group is each independently unsubstituted, or optionally substituted with 1 to 2 R²⁰);
(9-12-1)
   Y is benzene-1,2-diyl (wherein the group is substituted with one R²⁰).
(9-13)
   R¹¹ and R¹² are each independently a hydrogen atom, a deuterium atom, C₁₋₆ alkyl, C₁₋₆ haloalkyl, or C₃₋₆ cycloalkyl;
(9-13-1)
   R¹¹ and R¹² are hydrogen atoms.
(9-14)
   R¹³ and R¹⁴ are each independently a hydrogen atom, a deuterium atom, C₁₋₆ alkyl, C₁₋₆ haloalkyl, or C₃₋₆ cycloalkyl;
(9-14-1)
   R¹³ and R¹⁴ are each a hydrogen atom.
(9-15)
   R¹⁶ and R¹⁷ are each independently a hydrogen atom, a deuterium atom, C₁₋₆ alkyl, C₁₋₆ haloalkyl, or C₃₋₆ cycloalkyl;
(9-15-1)
   R¹⁶ and R¹⁷ are each a hydrogen atom.
(9-16)
   R¹⁸ and R¹⁹ are each independently a hydrogen atom, a halogen atom, or C₁₋₆ alkyl;
(9-16-1)
   R¹⁸ and R¹⁹ are each a hydrogen atom.
(9-17)
   R¹⁵ is each independently a hydrogen atom, C₁₋₆ alkyl substituted with 0 to 2 R^{a}, C₁₋₆ haloalkyl, or M^{a};
(9-17-1)
   R¹⁵ is 2-propyl, 1-propyl, trifluoromethyl, or M^{a}.
(9-18)
   R^{a} is independently C₁₋₆ alkyl, C₂₋₆ alkenyl, C₂₋₆ alkynyl, a halogen atom, C₁₋₆ haloalkyl, C₁₋₆ haloalkoxy, —CN, hydroxyl, —OM^{e}, —SM^{e}, —S(O)₂M^{e}, —C(O)NM^{f}M^{g}, —NM^{f}M^{g}, —N(M^{e})C(O)M^{h}, —N(M^{e})S(O)M^{h}, —N(M^{e})C(O)OM^{h}, —N(M^{e})C(O)NM^{f}M^{g}, or M^{b}.
(9-19)
   R²⁰ is independently a hydrogen atom, a halogen atom, —OH, —CN, —COOH, C₁₋₆ alkyl, C₁₋₆ alkoxy, C₁₋₆ haloalkoxy, C₂₋₁₀ alkoxyalkyl, C₄₋₂₀ alkoxyalkylalkynyl, C₂₋₁₀ haloalkoxyalkyl, C₁₋₆ hydroxyalkyl, C₃₋₁₀ hydroxyalkylalkynyl, C₂₋₁₀ hydroxyalkynyl, —B(R^{b})(R^{d}), —S(O)ₙ₁R^{c}, —N(R^{c})₂, —C(═O)N(R^{c})₂, —NHC(═O)R^{c}, —NHC(═O)OR^{c}, —NHC(═O)C(═O)N(R^{c})₂, —NHC(═O)C(═O)OR^{c}, —NHC(═O)N(R^{c})₂, —NHC(═O)NR^{c}C(═O)N(R^{c})₂, —NHC(═O)NR^{c}S(O)₂OR^{c}, —NHC(═O)NR^{c}S(O)₂N(R^{c})₂, —NHC(═S)N(R^{c})₂, —NHC(═NC≡N)NR^{c}, —NHC(═NC≡N)SR^{c}, —NHS(O)ₙ₁R^{c}, M^{c}, —(C₁₋₆ alkylene)-B(R^{b})(R^{d}), —(C₁₋₆ alkylene)-S(O)ₙ₁R^{c}, —(C₁₋₆ alkylene)-N(R^{c})₂, —(C₁₋₆ alkylene)-C(═O)N(R^{c})₂, —(C₁₋₆ alkylene)-NHC(═O)R^{c}, —(C₁₋₆ alkylene)-NHC(═O)OR^{c}, —(C₁₋₆ alkylene)-NHC(═O)C(═O)N(R^{c})₂, —(C₁₋₆ alkylene)-NHC(═O)C(═O)OR^{c}, —(C₁₋₆ alkylene)-NHC(═O)N(R^{c})₂, —(C₁₋₆ alkylene)-NHC(═O)NR^{c}C(═O)N(R^{c})₂, —(C₁₋₆ alkylene)-NHC(═O)NR^{c}S(O)₂OR^{c}, —(C₁₋₆ alkylene)-NHC(═O)NRCS(O)₂N(R^{c})₂, —(C₁₋₆ alkylene)-NHC(═S)N(R^{c})₂, —(C₁₋₆ alkylene)-NHC(═NC≡N)NR^{c}, (C₁₋₆ alkylene)-NHC(═NC≡N)SR^{c}, —(C₁₋₆ alkylene)-NHS(O)ₙ₁R^{c}, —(C₁₋₆ alkylene)-M^{c}, —CH≡CH—(C₁₋₆ alkyl), —CH═CH-M^{c}, —OM^{c}, —SM^{c}, or —N(R^{c})M^{c};
(9-19-1)
   R²⁰ is —NHC(O)NHCH₃, —NHC(O)C(CH₃)₂NH₂, —NHC(O)-(cyclobutane-1,1-diyl)-NH₂, or 1-methyl-4-pyrazolyl.
(9-20)
   R^{b} and R^{d} are each independently a hydrogen atom, hydroxyl, or C₁₋₆ alkyl;
(9-21)
   R^{c} is each independently a hydrogen atom, C₁₋₆ alkyl, C₆-10 aryl, 5- to 10-membered cyclic heteroaryl, a 3- to 10-membered cyclic non-aromatic heterocyclic group, C₃₋₁₀ cycloalkyl, or C₅₋₁₀ cycloalkenyl (wherein the group is each independently not substituted, or optionally substituted with 1 to 2 substituents selected from amino, hydroxy, methoxy, C₁₋₆ alkyl, C₃₋₁₀ cycloalkyl, and CN).
(9-22)
   M^{a}, M^{b}, and M^{c} are each independently C₆₋₁₀ aryl, C₅₋₁₀ heteroaryl, a C₃₋₁₀ non-aromatic heterocyclic group, C₃₋₁₀ cycloalkyl, or C₃₋₁₀ cycloalkenyl (wherein the groups are each independently not substituted, or optionally substituted with 1 to 2 M^{d});
(9-21-1)
   M^{a} is cyclopropyl.
(9-23)
   M^{d} is each independently C₁₋₆ alkyl, C₂₋₆ alkenyl, C₂₋₆ alkynyl, halogen, C₁₋₆ haloalkyl, —CN, oxo, —OM^{e}, —OC(O)M^{h}, —OC(O)NM^{f}M^{g}, —SM^{e}, —S(O)₂M^{e}, —S(O)₂NM^{f}M^{g}, —C(O)M^{e}, —C(O)-5 to 10-membered monocyclic cycloheteroaryl, —C(O)-5 to 10-membered monocyclic heteroaryl, —C(O)OM^{e}, —C(O)NM^{f}M^{g}, —NM^{f}M^{g}, —N(M^{e})C(O)M^{h}, —N(M^{e})S(O)₂M^{h}, —N(M^{e})C(O)OM^{h}, —N(M^{e})C(O)NM^{f}M^{g}, —(C₁₋₆ alkylene)OM^{e}, —(C₁₋₆ alkylene)-OC(O)M^{h}, —(C₁₋₆ alkylene)-OC(O)NM^{f}M^{g}, —(C₁₋₆ alkylene)-S(O)₂M^{e}, —(C₁₋₆ alkylene)-S(O)₂NM^{f}M^{g}, —(C₁₋₆ alkylene)-C(O)M^{c}, —(C₁₋₆ alkylene)-C(O)OM^{e}, —(C₁₋₆ alkylene)-C(O)NM^{f}M^{g}, —(C₁₋₆ alkylene)-NM^{f}M^{g}, —(C₁₋₆ alkylene)-N(M^{c})C(O)M^{h}, —(C₁₋₆ alkylene)-N(M^{e})S(O)₂M^{h}, —(C₁₋₆ alkylene)-N(M^{e})C(O)OM^{h}, —(C₁₋₆ alkylene)-N(M^{e})C(O)NM^{f}M^{g}, or (C₁₋₆ alkylene)-CN;
(9-24)
   W is independently a C₆₋₁₀ aromatic ring or a C₅₋₁₀ heteroaromatic ring (wherein the group is independently not substituted, or optionally substituted with 1 to 3 R²¹);
(9-24-1)
   W is benzene-1,2-diyl, thiophene-2,3-diyl, or pyridine-3,4-diyl (wherein the group is independently not substituted, or optionally substituted with 1 to 2 R²¹)
(9-25)
   R²¹ is each independently C₁₋₆ alkyl, a halogen atom, C₁₋₆ haloalkyl, C₁₋₆ haloalkoxy, C₁₋₆ cycloalkyl, —OM^{e}, —OC(O)M^{h}, —OC(O)NM^{f}M^{g}, —SM^{e}, —S(O)₂M^{e}, —S(O)₂NM^{f}M^{g}, —C(O)M^{e}, —C(O)OM^{e}, —C(O)NM^{f}M^{g}, —N(M^{e})C(O)M^{h}, —N(M^{e})S(O)M^{h}, —N(M^{e})C(O)OM^{h}, or —N(M^{e})C(O)NM^{f}M^{g};
(9-25-1)
   R²¹ is a halogen atom or methyl.
(9-26)
   M^{e}, M^{f}, and M^{g} are each independently a hydrogen atom, C₁₋₆ alkyl, C₁₋₆ haloalkyl, or C₃₋₆ cycloalkyl.
(9-27)
   M^{h} is each independently C₁₋₆ alkyl, C₁₋₆ haloalkyl, or C₃₋₆ cycloalkyl.
(9-28)
   n1 and n2 are, for each instance, independently 0, 1, or 2;
(9-27-1)
   n1 is 1, and n2 is 1.
(9-29)
   n3 and n4 are, for each instance, independently 0, 1, 2, or 3;
(9-29-1)
   n3 is 1 or 2, and n4 is 1.

In formula (9), variables can be 9-1-1, 9-3-1, 9-4-1, 9-6-1, 9-7-1, 9-8-1, 9-12-1, 9-13-1, 9-14-1, 9-15-1, 9-16-1, 9-17-1, 9-19-1, 9-24-1, 9-25-1, and 9-29-1.

While the preferred variables in the compound of the present disclosure represented by formula (10) are described below, the technical scope of the present disclosure is not limited to the scope of the compounds described below.

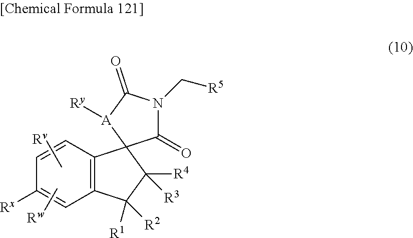

(10-1)
   A is independently selected from O, N, and S;
(10-1-1)
   A is O.
(10-2)
   R^{y} is absent, a hydrogen atom, alkyl, substituted alkyl, or alkenyl;
(10-2-1)
   R^{y} is absent.
(10-3)
   R^{v}, R^{w}, and R^{x} are each independently a hydrogen atom, a halogen atom, cyano, nitro, alkyl, substituted alkyl, alkenyl, alkynyl, cycloalkyl, substituted cycloalkyl, a heterocycle, a substituted heterocycle, aryl, substituted aryl, an aromatic heterocycle, a substituted aromatic heterocycle, substituted amide, substituted guanidino, substituted urea, amino, substituted amino, alkoxy, or substituted alkoxy;
(10-3-1)
   R^{v} is a hydrogen atom or a halogen atom, R^{w} is a hydrogen atom, and R^{x} is —NHC(O)NHCH₃ or 3,5-dimethyl-4-isooxazolyl.
(10-4)
   R¹, R², R³, and R⁴ are each independently a hydrogen atom, alkyl, or a halogen atom;
   wherein R¹ and R², R² and R³, or R³ and R⁴ together may from a ring;
(10-4-1)
   R¹ and R² are each a hydrogen atom, or R¹ and R² together form a cyclopropyl ring, and R³ and R⁴ are each a hydrogen atom.
(10-4)
   R⁵ is alkyl, alkoxy, amino, substituted amino, amide, substituted amide, ester, carbonyl, a heterocycle, or a substituted heterocycle;
(10-4-1)
   R⁵ is

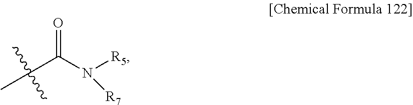

R₆ is 6-fluorophenylmethyl, and R⁷ is 1,1,1-trifluoro-2-propyl, or
R₆ and R⁷, together with a nitrogen atom, form

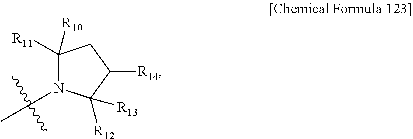

R₁₀ and R₁₁ are each independently a hydrogen atom, methyl, ethyl, methoxymethyl, or cyclopropyl, R₁₄ is a hydrogen atom, and R₁₂ and R₁₃ are each independently 4-fluorophenyl, 4-fluoro-2-methylphenyl, 3,4-difluorophenyl, 3,4,5-trifluorophenyl, 3,3-difluorocyclohexyl, 3-chloro, 4-fluorophenyl, 2,4-difluorophenyl, 2-methyl-4-chlorophenyl, 2-methyl-4-fluorophenyl, or cyclohexyl.

In formula (10), variables can be 10-1-1, 10-2-1, 10-3-1, and 10-4-1.

While the preferred variables in the compound of the present disclosure represented by formula (11) are described below, the technical scope of the present disclosure is not limited to the scope of the compounds described below.

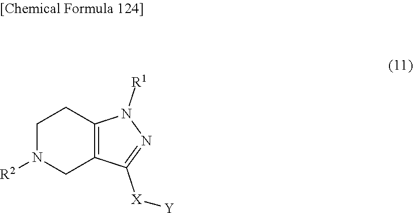

(11-1)
   R¹ is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R¹ is optionally substituted with one or more R^{d};
(11-1-1)
   R¹ is 1-methylcarbonyl-4-piperidinyl or 4-tetrahydropyranyl.
(11-2)
   R² is —C(O)—N(R^{e})₂, —S(O)—N(R^{e})₂, —S(O)₂—N(R^{e})₂, —C(O)—R⁶, —C(O)—O—(R^{e}), —S(O)—R^{e}, or —S(O)₂—R^{e};
(11-2-1)
   R² is —C(O)NHCH₃ or C(O)CH₃.
(11-3)
   X is absent, —C(O), or C₁₋₃ alkyl;
   Y is phenyl, a 9-membered bicyclic carbocyclic ring, a 10-membered bicyclic carbocyclic ring, a 9-membered bicyclic heterocycle, or a 10-membered bicyclic heterocycle;
   wherein Y is optionally substituted with R^{a}, and Y is optionally further substituted with one or more R^{b}; or
   X combined with Y is selected from the group consisting of

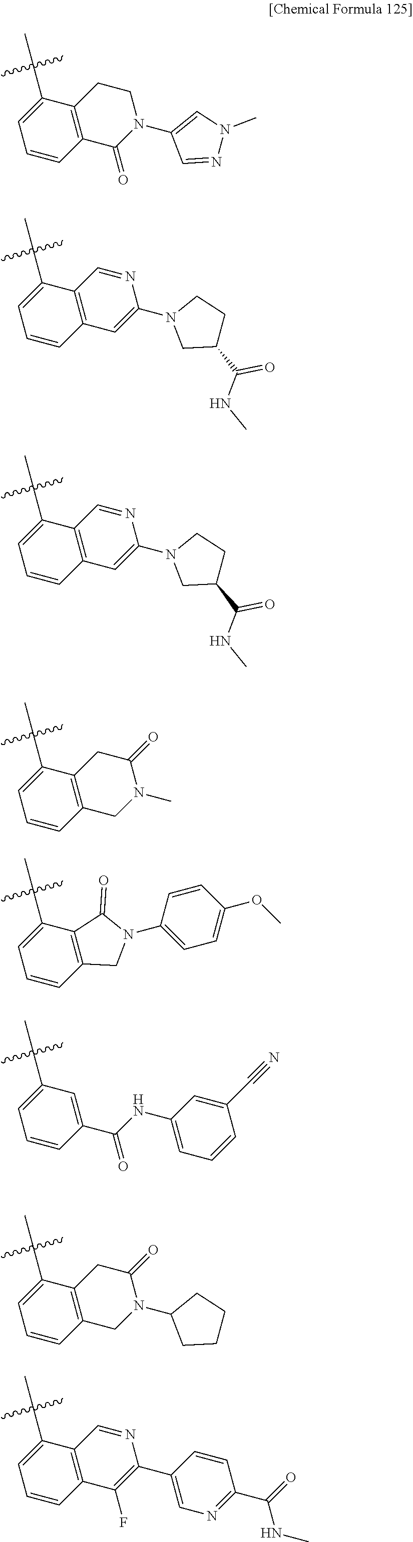

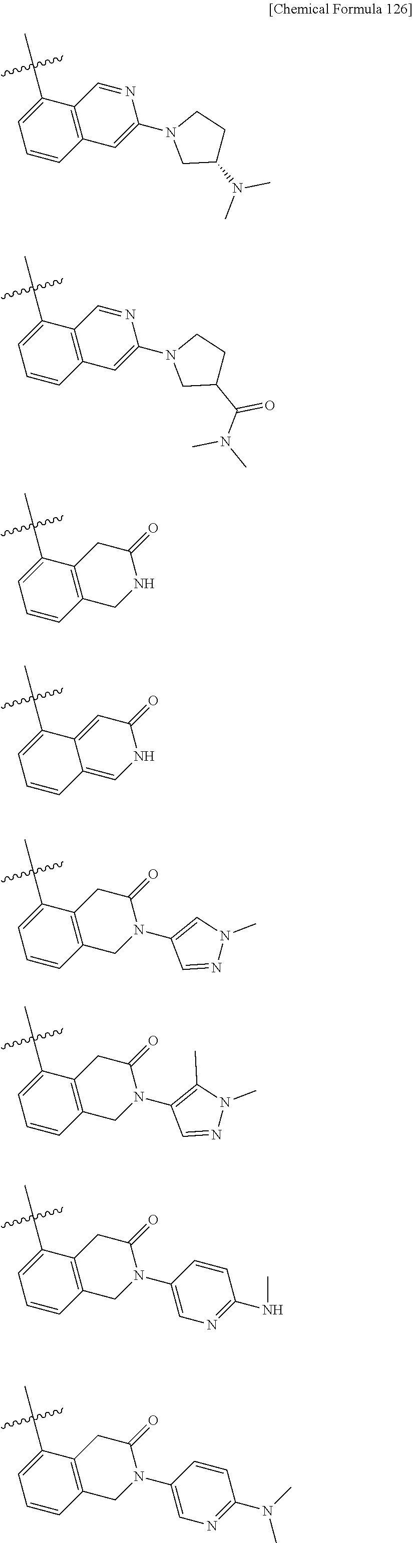

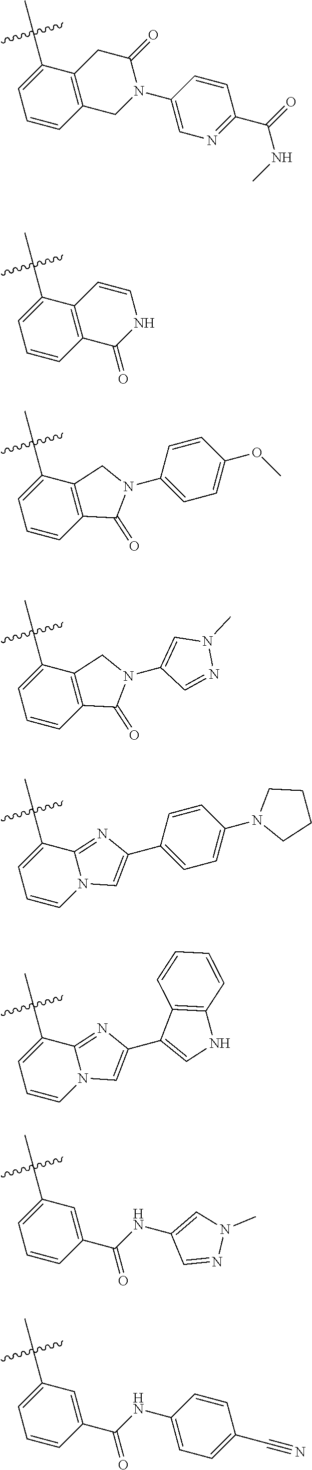

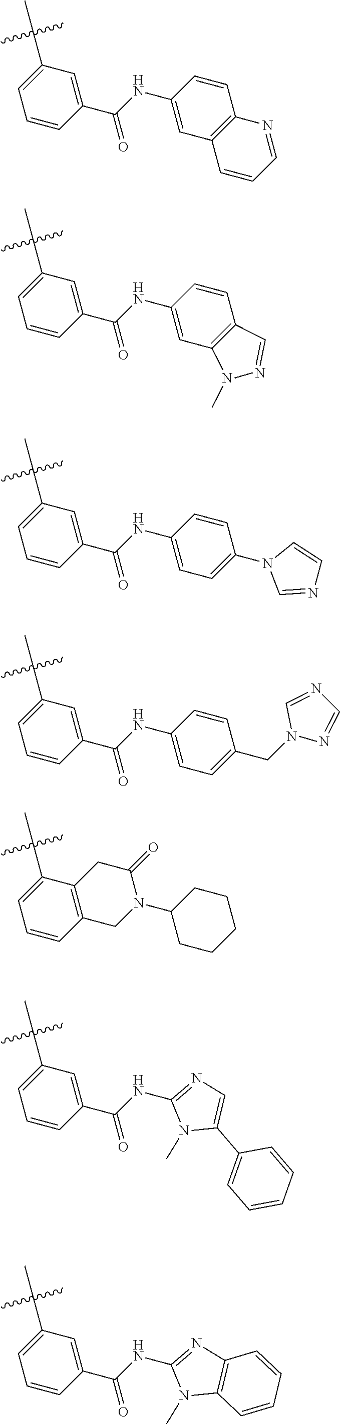

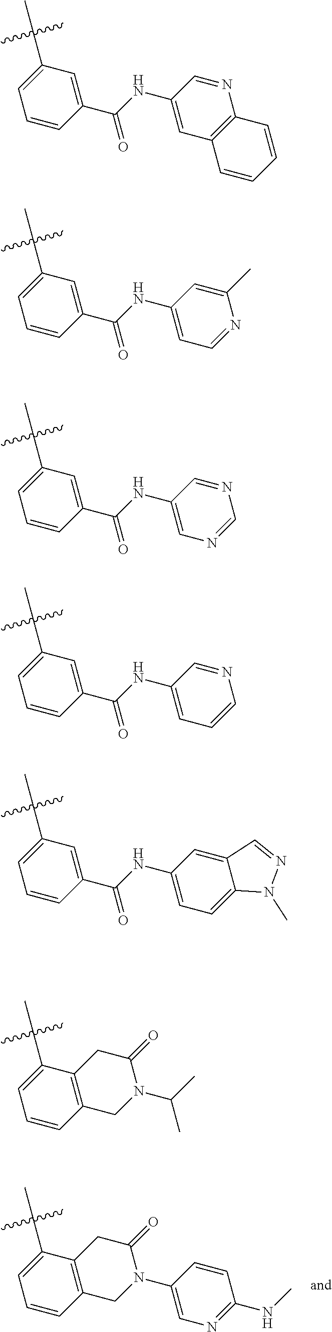

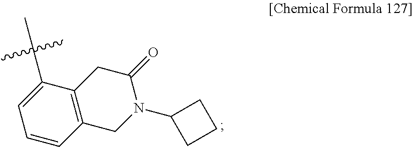 [SMILES: *c1cccc2c1CC(=O)N(C1CCC1)C2]

(11-3-1)
   X combined with Y is selected from the group consisting of

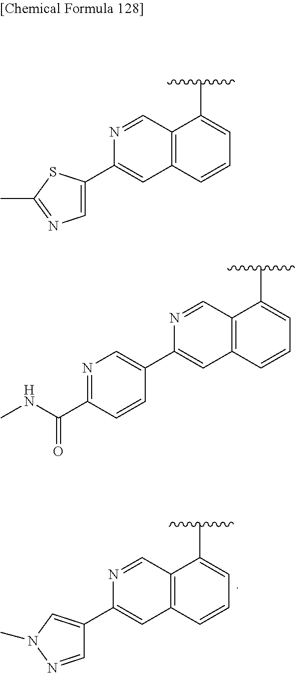 [SMILES: *c1cccc2cc(-c3cnc(C)s3)ncc12 ; *c1cccc2cc(-c3ccc(C(=O)NC)nc3)ncc12 ; *c1cccc2cc(-c3cnn(C)c3)ncc12]

(11-4)
   each R^{a} is independently selected from the group consisting of a 5-membered carbocyclic ring, a 6-membered carbocyclic ring, a 5-membered heterocycle, and a 6-membered heterocycle, wherein the 5-membered carbocyclic ring, 6-membered carbocyclic ring, 5-membered heterocycle, and 6-membered heterocycle are optionally substituted with one or more R^{c};
   each R^{b} is independently selected from the group consisting of a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarboryl, C₁₋₄ alkanoyl, —C(O)—N(R^{f})₂, —N(R^{f})C(O)—R^{f}, and C₁₋₄ alkanoyloxy, wherein each of C₂₋₄ alkyl, C₂₋₄ alkenyl, C₁₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₄ alkoxy, and C₁₋₃ alkyl that is optionally substituted with one or more groups independently selected from halogen;
   R^{c} is independently selected from the group consisting of a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl) C₁₋₄ alkyl, C₁₋₄ alkoxy, C₁₋₄ alkoxycarbonyl, C₁₋₄ alkanoyl, and C₁₋₄ alkanoyloxy, wherein each of C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₃ alkoxy, and C₁₋₃ alkyl, and C₁₋₃ alkyl that is optionally substituted with one or more groups independently selected from halogen;
   each R^{d} is independently selected from the group consisting of oxo, a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy, wherein each of C₁₋₄ alkyl, C₂₋₄ alkenyl, C₂₋₄ alkynyl, C₂₋₆ cycloalkyl, (C₂₋₆ cycloalkyl) C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₃ alkoxy, and C₁₋₃ alkyl that is optionally substituted with one or more groups independently selected from halogen;
   each R^{e} is independently selected from a hydrogen atom, C₁₋₄ alkyl, C₁₋₄ alkenyl, C₁₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkyl, and C₁₋₃ alkyl that is optionally substituted with one or more groups independently selected from halogen; and
   each R^{f} is a hydrogen atom or C₁₋₄ alkyl; or
selected from the group consisting of

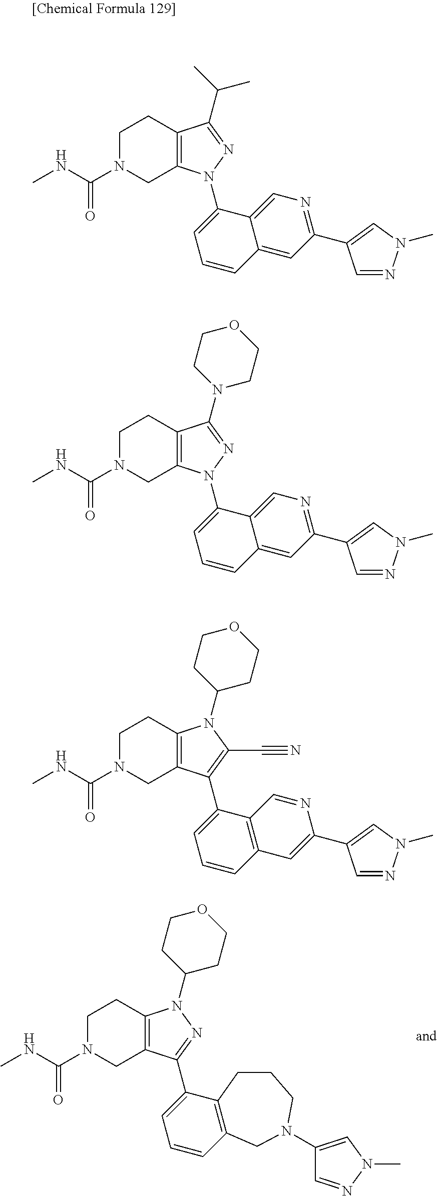

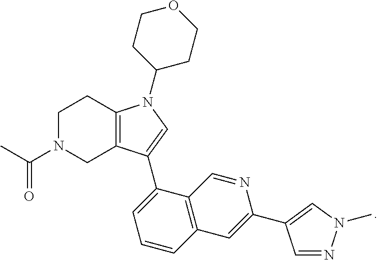 [SMILES: CC(=O)N1CCc2c(c(-c3cccc4cc(-c5cnn(C)c5)ncc34)cn2C2CCOCC2)C1]

In formula (11), variables can be 11-1-1, 11-2-1, and 11-3-1.

While the preferred variables in the compound of the present disclosure represented by formulas (12) and (13) are described below, the technical scope of the present disclosure is not limited to the scope of the compounds described below.

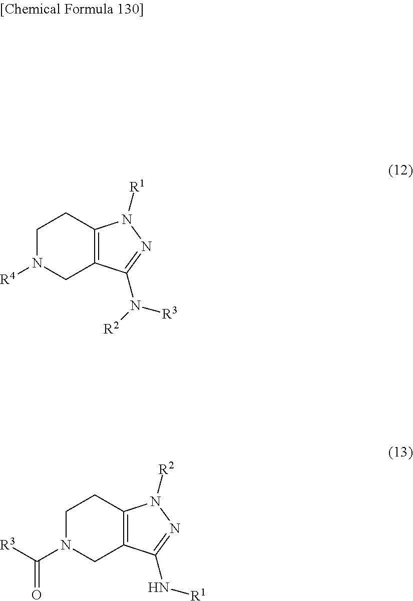

(12-1)
   R¹ in formula (12) is C₁₋₁₂ alkyl, C₂₋₁₂ alkenyl, C₂₋₁₂ alkynyl, a 3- to 12-membered carbocyclic ring, or a 3- to 12-membered heterocycle, wherein C₁₋₁₂ alkyl, C₂₋₁₂ alkenyl, C₂₋₁₂ alkynyl, 3- to 12-membered carbocyclic ring, and 3- to 12-membered heterocycle of R¹ is optionally substituted with one or more R^{b};
(12-1-1)
   R¹ in formula (12) is 4-oxanyl, 1,1-dioxo-4-thianyl, 1-methylcarbonyl-4-piperidinyl, 1,1,1-trifluoroethyl-4-piperidinyl, 1,1-difluoroethyl-4-piperidinyl, 2-methyl-4-oxanyl, 1,1-difluoro-4-cyclohexanyl, 1-methylsulfonyl-4-piperidinyl, 1-cyanomethyl-4-piperidinyl, 1-cyclopropylcarbonyl-4-piperidinyl, 2-propyl, 4-oxepanyl, 2-cyclopropylethyl, 4-methoxycyclohexyl, or 4-cyanocyclohexyl.
(12-2)
   R² in formula (12) is selected from C₆₋₂₀ aryl, C₁₋₂₀ heteroaryl, —(C₆₋₂₀ aryl)-(C₁₋₂₀ heteroaryl), —(C₁₋₂₀ heteroaryl)-(C₆₋₂₀ aryl), and —(C₁₋₂₀ heteroaryl)-(C₁₋₂₀ heteroaryl), wherein each of C₆₋₂₀ aryl, C₁₋₂₀ heteroaryl, —(C₆₋₂₀ aryl)-(C₁₋₂₀ heteroaryl), and (C₁₋₂₀ heteroaryl)-(C₁₋₂₀ heteroaryl) is independently optionally substituted with one or more substituents independently selected from R⁷, oxo, fluorine, chlorine, bromine, iodine, —NO₂, —N(R^{a})₂, —CN, —C(O)—N(R^{a})₂, —S(O)—N(R^{a})₂, —S(O)₂—N(R^{a})₂, —O—R^{a}, —S—R^{a}, —O—C(O)—R^{a}, —O—C(O)—O—R^{a}, —C(O)—R^{a}, —C(O)—O—R^{a}, —S(O)—R^{d}, —S(O)₂—R^{a}, —O—C(O)—N(R^{a})₂, —N(R^{a})—C(O)—OR^{a}, —N(R^{a})—C(O)—N(R^{a})₂, —N(R^{a})—C(O)—R^{a}, —N(R^{a})—S(O)—R^{a}, —N(R^{a})—S(O)₂—R^{a}, —N(R^{a})—S—(O)—N(R^{a})₂, and —N(R^{a})—S(O)₂—N(R^{a})₂;
   R³ in formula (12) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R³ is optionally substituted with one or more R^{e}; or
   R² and R³ in formula (12), together with the nitrogen to which they are attached, form a 3- to 12-membered heterocycle optionally substituted with one or more R^{e};
(12-2-1)
   R² and R³ in formula (12), together with the nitrogen to which they are attached, form a group selected from the group consisting of

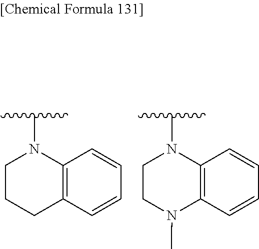

wherein the group is optionally substituted with one or more R^{e}.
(12-3)
   R⁴ in formula (12) is C₁₋₄ alkyl, C₂₋₄ alkenyl, C₂₋₄ alkynyl, a 3- to 5-membered carbocyclic ring, a 3- to 5-membered heterocycle, —C(O)—N(R^{h})₂, —S(O)—N(R^{h})₂, —S(O)₂—N(R^{h}), —C(O)—R^{h}, —C(O)—OR^{h}, —S(O)—R^{h}, or —S(O)—R^{a}, wherein any of C₁₋₄ alkyl, C₂₋₄ alkenyl, C₂₋₄ alkynyl, 3- to 5-membered carbocyclic ring, and 3- to 5-membered heterocycle is optionally substituted with one or more substituents independently selected from fluorine, chlorine, bromine, iodine, a 3- to 5-membered carbocyclic ring, —C(O)—N(R^{h})₂, —S(O)—N(R^{h})₂, —S(O)₂—N(R^{h})₂, —O—R^{h}, —S—R^{h}, —O—C(O)—R^{h}, —O—C(O)—O—R^{h}, —C(O)—R^{h}, —C(O)—O—R^{h}, —S(O)—R^{h}, —S(O)₂—R^{h}, —O—C(O)—N(R^{h})₂, —N(R^{h})—C(O)—OR^{h}, —N(R^{h})—C(O)—N(R^{h})₂, —N(R^{h})—C(O)—R^{h}, —N(R^{h})—S(O)—R^{h}, —N(R^{h})—S(O)₂—R^{h}, —N(R^{h})—S(O)—N(R^{h})₂, and —N(R^{h})—S(O)₂—N(R^{h})₂;
(12-3-1)
   R⁴ in formula (12) is methylcarbonyl, methylaminocarbonyl, or aminocarbonyl.
(12-4)
   each R^{a} in formula (12) is independently selected from a hydrogen atom, C₁₋₆ alkyl, C₂₋₆ alkenyl, a C₂₋₆ alkynyl, a carbocyclic ring, and a heterocycle, wherein each of C₁₋₆ alkyl, C₂₋₆ alkenyl, C₂₋₆ alkynyl, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
   two R³, together with the nitrogen to which they are attached, form a heterocycle optionally substituted with one or more groups independently selected from oxo, halogen, and C₁₋₃ alkyl that is optionally substituted with one or more groups independently selected from oxo and halogen.
(12-5)
   each R^{b} in formula (12) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —O—C(O)—O—R^{c}, —C(O)—R^{c}, —C(O)—O—R^{c}, —S(O)—R^{c}, —S(O)₂—R^{c}, —O—C(O)—N(R^{c})₂, —N(R^{c})—C(O)—OR^{c}, —N(R^{c})—C(O)—N(R^{c})₂, —N(R^{c})—C(O)—R^{c}, —N(R^{c})—S(O)—R^{c}, —N(R^{c})—S(O)₂—R^{c}, —N(R^{c})—S(O)—N(R^{c})₂, and —N(R^{c})—S(O)₂—N(R^{c})₂, wherein any of C₁₋₆ alkyl, C₂₋₆ alkenyl, C₂₋₆ alkynyl, carbocyclic ring, heterocycle, aryl, and heteroaryl is optionally substituted with one or more groups independently selected from oxo, halogen, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —C(O)—R^{c}, —C(O)—O—R^{c}, —S(O)—R^{e}, —S(O)₂—R^{c}, —C(O)—N(R^{c})₂, —N(R^{c})—C(O)—R^{c}, —N(R^{c})—S(O)—R^{c}, —N(R^{c})—S(O)₂—R^{c}, and C₁₋₆ alkyl that is optionally substituted with one or more groups independently selected from oxo and halogen.
(12-6)
   each R^{c} of formula (12) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —O—C(O)—R^{d}, —C(O)—R^{d}, —C(O)—O—R^{d}, —S(O)—R^{d}, —S(O)₂—R^{d}, —C(O)—N(R^{d})₂, —N(R^{d})—C(O)—R^{d}, —N(R^{d})—S(O)—R^{d}, —N(R^{d})—S(O)₂—R^{d}, and C₁₋₆ alkyl, and the carbocyclic ring and C₁₋₆ alkyl are optionally substituted with one or more groups independently selected from oxo, halogen, C₁₋₆ alkyl, cyano, —N(R^{d}), —O—R^{d}, a heterocycle, and a carbocyclic ring that is optionally substituted with one or more groups independently selected from halogen and C₁₋₆ alkyl.
(12-7)
   each R^{d} in formula (12) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is independently optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or,
   two R^{d}, together with the nitrogen to which they are attached, form a heterocycle optionally substituted with one or more groups independently selected from oxo, halogen, and C₁₋₃ alkyl that is optionally substituted with one or more groups independently selected from oxo and halogen.
(12-8)
   each R^{e} in formula (12) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{f})₂, —CN, —C(O)—N(R^{f})₂, —S(O)—N(R^{f})₂, —S(O)₂—N(R^{f})₂, —O—R^{f}, —S—R^{f}, —O—C(O)—R^{f}, —O—C(O)—O—R^{f}, —C(O)—R^{f}, —C(O)—O—R^{f}, —S(O)—R^{f}, —S(O)₂—R^{f}, —O—C(O)—N(R^{f})₂, —N(R^{f})—C(O)—OR^{f}, —N(R^{f})—C(O)—N(R^{f})₂, —N(R^{f})—C(O)—R^{f}, —N(R^{f})—S(O)—R^{f}, —N(R^{f})—S(O)₂—R^{f}, —N(R^{f})—S(O)—N(R^{f})₂, and —N(R^{f})—S(O)₂—N(R^{f})₂, wherein any of C₁₋₆ alkyl, C₂₋₆ alkenyl, C₂₋₆ alkynyl, carbocyclic ring, heterocycle, aryl, and heteroaryl is optionally substituted with one or more groups independently selected from oxo, halogen, —NO₂, —N(R^{f})₂, —CN, —C(O)—N(R^{f})₂, —S(O)—N(R^{f})₂, —S(O)₂—N(R^{f})₂, —O—R^{f}, —S—R^{f}, —O—C(O)—R^{f}, —C(O)—R^{f}, —C(O)—O—R^{f}, —S(O)—R^{f}, —S(O)₂—R^{f}, —C(O)—N(R^{f}), —N(R^{f})—C(O)—R^{f}, —N(R^{f})—S(O)—R^{f}, and —N(R^{f})—S(O)₂—R^{f}, a carbocyclic ring, and C₁₋₆ alkyl that is optionally substituted with one or more groups independently selected from oxo and halogen;
(12-8-1)
   each R^{e} in formula (12) is methyl, difluoromethyl, 2-methoxy-4-pyridyl, 1-methyl-4-pyrazolyl, 6-methylcarbonylamino-3-pyridinyl, trifluoromethyl, cyano, 1,5-dimethyl-4-pyrazolyl, or 2-thiophenyl.
(12-9)
   each R^{f} in formula (12) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{g})₂, —CN, —C(O)—N(R^{g})₂, —S(O)—N(R^{g})₂, —S(O)₂—N(R^{g})₂, —O—R^{g}, —S—R^{g}, —O—C(O)—R^{g}, —C(O)—R^{g}, —C(O)—O—R^{g}, —S(O)—R^{g}, —S(O)₂—R^{g}, —C(O)—N(R^{e})₂, —N(R^{g})—C(O)—R^{g}, —N(R^{g})—S(O)—R^{g}, —N(R^{g})—S(O)₂—R^{g}, and C₁₋₆ alkyl, and the carbocyclic ring and C₁₋₆ alkyl are optionally substituted with one or more groups independently selected from oxo, halogen, C₁₋₆ alkyl, cyano, —N(R^{g})₂, —O—R^{g}, a heterocycle, and a carbocyclic ring that is optionally substituted with one or more groups independently selected from halogen and C₁₋₆ alkyl.
(12-10)
   each R^{g} in formula (12) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
   two R^{g}, together with the nitrogen to which they are attached, form a heterocycle optionally substituted with one or more groups independently selected from oxo, halogen, and C₁₋₃ alkyl that is optionally substituted with one or more groups independently selected from oxo and halogen.
(12-11)
   each R^{h} in formula (12) is independently selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkoxy, and C₁₋₃ alkyl that is optionally substituted with one or more groups independently selected from halogen.
(13-1)
   R¹ in formula (13) is selected from C₆₋₂₀ aryl, C₁₋₂₀ heteroaryl, —(C₆₋₂₀ aryl)-(C₁₋₂₀ heteroaryl), and —(C₁₋₂₀ heteroaryl)-(C₁₋₂₀ heteroaryl), wherein each of C₆₋₂₀ aryl, C₁₋₂₀ heteroaryl, —(C₆₋₂₀ aryl)-(C₁₋₂₀ heteroaryl), and (C₁₋₂₀ heteroaryl)-(C₁₋₂₀ heteroaryl) is independently optionally substituted with one or more substituents independently selected from R^{c}, oxo, fluorine, chlorine, bromine, iodine, —NO₂, —N(R^{a})₂, —CN, —C(O)—N(R^{a})₂, —S(O)—N(R^{a})₂, —S(O)₂—N(R^{a})₂, —O—R^{a}, —S—R^{a}, —O—C(O)—R^{a}, —O—C(O)—O—R^{a}, —C(O)—R^{a}, —C(O)—O—R^{a}, —S(O)—R^{a}, —S(O)₂—R^{a}, —O—C(O)—N(R^{a})₂, —N(R^{a})—C(O)—OR^{a}, —N(R^{a})—C(O)—N(R^{a})₂, —N(R^{a})—C(O)—R^{a}, —N(R^{a})—S(O)—R^{a}, —N(R^{a})—S(O)₂—R^{a}, —N(R^{a})—S(O)—N(R^{a})₂, and —N(R^{a})—S(O)₂—N(R^{a})₂;
(13-1-1)
   R¹ in formula (13) is -(phenylene)-(pyrazole), wherein the group is optionally substituted with one or more R^{e}.
(13-2)
   R² in formula (13) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R² is optionally substituted with one or more R^{b};
(13-2-1)
   R² in formula (13) is 3-oxolanyl.
(13-3)
   R³ in formula (13) is C₁₋₄ alkyl, C₂₋₄ alkenyl, C₂₋₄ alkynyl, a 3- to 5-membered carbocyclic ring, a 3- to 5-membered heterocycle, —C(O)—N(R^{e})₂, —S(O)—N(R^{e})₂, —S(O)₂—N(R^{e})₂, —C(O)—R^{e}, —C(O)—OR^{e}, —S(O)—R^{e}, or —S(O)₂—R^{e}, wherein any of C₁₋₄ alkyl, C₂₋₄ alkenyl, C₂₋₄ alkynyl, 3- to 5-membered carbocyclic ring, and 3- to 5-membered heterocycle is optionally substituted with one or more substituents independently selected from fluorine, chlorine, bromine, iodine, a 3- to 5-membered carbocyclic ring, —C(O)—N(R^{e})₂, —S(O)—N(R^{c})₂, —S(O)₂—N(R^{c})₂, —O—R^{c}, —S—R^{c}, —O—C(O)—R^{c}, —O—C(O)—O—R^{e}, —C(O)—R, —C(O)—O—R^{e}, —S(O)—R^{e}, —S(O)₂—R^{e}, —O—C(O)—N(R^{c})₂, —N(R^{c})—C(O)—OR^{c}, —N(R^{c})—C(O)—N(R^{c})₂, —N(R^{c})—C(O)—R^{e}, —N(R^{e})—S(O)—R^{e}, —N(R^{e})—S(O)₂—R^{e}, —N(R^{e})—S(O)—N(R^{e})₂, and —N(R^{c})—S(O)₂—N(R^{c})₂;
(13-3-1)
   R³ in formula (13) is CH₃.
(13-4)
   each R^{a} in formula (13) is independently selected from a hydrogen atom, C₁₋₆ alkyl, C₂₋₆ alkenyl, C₂₋₆ alkynyl, a carbocyclic ring, and a heterocycle, wherein each of C₁₋₆ alkyl, C₂₋₆ alkenyl, C₂₋₆ alkynyl, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
   two R^{a}, together with the nitrogen to which they are attached, form a heterocycle optionally substituted with one or more groups independently selected from oxo, halogen, and C₁₋₃ alkyl that is optionally substituted with one or more groups independently selected from oxo and halogen.
(13-5)
   each R^{b} in formula (13) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —O—C(O)—O—R^{c}, —C(O)—R^{c}, —C(O)—O—R^{c}, —S(O)—R^{c}, —S(O)₂—R^{c}, —O—C(O)—N(R^{c})₂, —N(R^{c})—C(O)—OR^{c}, —N(R^{c})—C(O)—N(R^{c})₂, —N(R^{c})—C(O)—R^{c}, —N(R^{c})—S(O)—R^{c}, —N(R^{c})—S(O)₂—R^{c}, —N(R^{c})—S(O)—N(R^{c})₂, and —N(R^{c})—S(O)₂—N(R^{c})₂, wherein any of C₁₋₆ alkyl, C₂₋₆ alkenyl, C₂₋₆ alkynyl, carbocyclic ring, heterocycle, aryl, and heteroaryl is optionally substituted with one or more groups independently selected from oxo, halogen, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —C(O)—R^{c}, —C(O)—O—R^{c}, —S(O)—R^{c}, —S(O)₂—R^{c}, —C(O)—N(R^{c})₂, —N(R^{c})—C(O)—R^{c}, —N(R^{c})—S(O)—R^{c}, —N(R^{c})—S(O)₂—R^{c}, and C₁₋₆ alkyl that is optionally substituted with one or more groups independently selected from oxo and halogen.
(13-6)
   each R^{c} in formula (13) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —O—C(O)—R^{d}, —C(O)—R^{d}, —C(O)—O—R^{d}, —S(O)—R^{d}, —S(O)₂—R^{d}, —C(O)—N(R^{d})₂, —N(R^{d})—C(O)—R^{d}, —N(R^{d})—S(O)—R^{d}, —N(R^{d})—S(O)₂—R^{d}, and C₁₋₆ alkyl, and the carbocyclic ring and C₁₋₆ alkyl are optionally substituted with one or more groups independently selected from oxo, halogen, C₁₋₆ alkyl, cyano, —N(R^{d})₂, —O—R^{d}, a heterocycle, and a carbocyclic ring that is optionally substituted with one or more groups independently selected from halogen and C₁₋₆ alkyl.
(13-7)
   each of R^{d} in formula (13) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
   two R^{d}, together with the nitrogen to which they are attached, form a heterocycle optionally substituted with one or more groups independently selected from oxo, halogen, and C₁₋₃ alkyl that is optionally substituted with one or more groups independently selected from oxo and halogen.
(13-8)
   each R^{e} in formula (13) is selectively selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkoxy, and C₁₋₃ alkyl that is optionally substituted with one or more groups independently selected from halogen;
(13-8-1)
   each R^{e} in formula (13) is methyl or fluorine.

In formula (12), variables can be 12-1-1, 12-2-1, 12-3-1, and 12-8-1.

In formula (13), variables can be 13-1-1, 13-2-1, 13-3-1, and 13-8-1.

While the preferred variables in the compound of the present disclosure represented by formula (14) are described below, the technical scope of the present disclosure is not limited to the scope of the compounds described below.

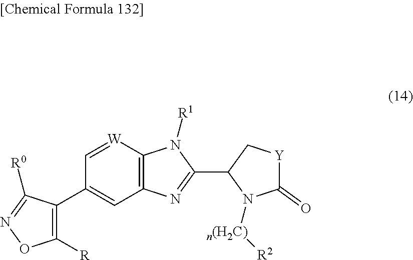

(14-1)
   R⁰ and R are the same or different, each a hydrogen atom, or C₁₋₆ alkyl, which is unsubstituted or substituted with OH, —OC(O)R′, or OR′ (wherein R′ is unsubstituted C₁₋₆ alkyl);
(14-1-1)
   R⁰ and R are each methyl.
(14-2)
   W is N or CH;
(14-2-1)
   W is CH.
(14-3)
   R¹ is an unsubstituted or substituted group, which is C-linked 4- to 6-membered heterocyclyl, C₃₋₆ cycloalkyl, or C₁₋₆ alkyl that is unsubstituted or substituted with C₆₋₁₀ aryl, 5- to 12-membered N-containing heteroaryl, C₃₋₆ cycloalkyl, OH, —OC(O)R′, or OR′ (wherein R′ is as defined above, or a group represented by:

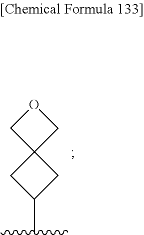 [SMILES: *C1CC2(COC2)C1]

(14-3-1)
   R¹ is 4-methoxycyclohexyl.
(14-4)
   Y is —CH₂—, —CH₂CH₂—, or CH₂CH₂CH₂—;
(14-4-1)
   Y is —CH₂—.
(14-5)
   n is 0 or 1;
(14-5-1)
   n is 0.
(14-6)
   R² is a group selected from C₆₋₁₀ aryl, 5- to 12-membered N-containing heteroaryl, C₃₋₆ cycloalkyl, and C₅₋₆ cycloalkenyl, which are unsubstituted or substituted, and the C₆₋₁₀ aryl may be fused to a 5- or 6-membered heterocycle;
(14-6-1)
   R² is 3,4-difluorophenyl.

In formula (14), variables can be 14-1-1, 14-2-1, 14-3-1, 14-4-1, 14-5-1, and 14-6-1.

While the preferred variables in the compound of the present disclosure represented by formula (15) are described below, the technical scope of the present disclosure is not limited to the scope of the compounds described below.

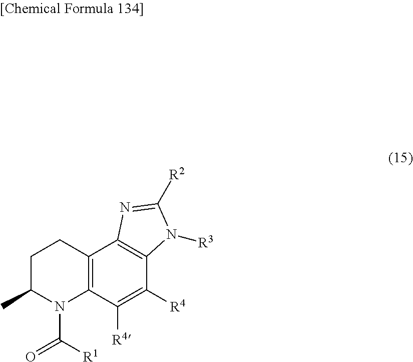

(15-1)
   R¹ is —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, or OR⁵;
(15-1-1)
   R¹ is methyl.
(15-2)
   R² is hydrogen, —C₁₋₆ alkyl, —C₂₋₆ alkenyl, C₂₋₆ alkynyl, —C₃₋₈ cycloalkyl, —C₄₋₈ cycloalkenyl, heterocyclyl, heteroaryl, or aryl, wherein each of alkyl, alkenyl, alkynyl, cycloalkyl, cycloalkenyl, heterocyclyl, heteroaryl, and aryl is optionally substituted with one or more R⁶, and a —C₁₋₆ alkyl group has one or more methylene units optionally substituted with —NR⁶—, —O—, or —S—;
(15-2-1)
   R² is methyl substituted with phenyl, wherein the phenyl is optionally substituted with one or more R¹⁰, and the methyl is optionally substituted with one or more R⁶.
(15-3)
   R³ is hydrogen, —C₁₋₆ alkyl, —C₂₋₆ alkenyl, —C₂₋₆ alkynyl, —C₃₋₈ cycloalkyl, —C₄₋₈ cycloalkenyl, spirocycloalkyl, spiroheterocyclyl, heterocyclyl, heteroaryl, or aryl, and each of alkyl, alkenyl, alkynyl, cycloalkyl, cycloalkenyl, spirocycloalkyl, spiroheterocyclyl, heterocyclyl, heteroaryl, and aryl is optionally substituted with one or more R⁷;
(15-3-1)
   R³ is cyclohexyl optionally substituted with one or more R⁷.
(15-4)
   R⁴ and R^{4′} are each independently —H, halogen, —OH, —CN, or NH₂;
(15-4-1)
   R⁴ and R^{4′} are each —H.
(15-5)
   R⁵ is —C₁₋₆ alkyl, —C₃₋₈ cycloalkyl, heterocyclyl, aryl, or heteroaryl.
(15-6)
   R⁶ and R⁷ are each independently, for each instance, hydrogen, —C₁₋₆ alkyl, —C₃₋₈ cycloalkyl, —C₄₋₈ cycloalkenyl, heterocyclyl, aryl, spirocycloalkyl, spiroheterocyclyl, heteroaryl, —OH, halogen, oxo, —CN, —SR⁸, —OR⁸, —(CH₂)ₙ—OR⁸, —NHR⁸, —NR⁸R⁹, —S(O)₂NR⁸R⁹, —S(O)₂R^{8′}, —C(O)R^{8′}, —C(O)OR⁸, —C(O)NR⁸R⁹, —NR⁸C(O)R^{9′}, —NR⁸S(O)₂R^{9′}, —S(O)R^{8′}, —S(O)NR⁸R⁹, or NR⁸S(O)R^{9′}, wherein each of alkyl, cycloalkyl, heterocyclyl, spirocycloalkyl, spiroheterocyclyl, heteroaryl, and aryl is optionally substituted with one or more R¹⁰;
   wherein any two R⁶ or any two R⁷, when on non-adjacent atoms, can attach and form bridged cycloalkyl or heterocyclyl, wherein any two R⁶ or any two R⁷, when on adjacent atoms, can attach and form cycloalkyl, heterocyclyl, aryl, or heteroaryl;
(15-6-1)
   R⁶ is methyl, and R⁷ is —C(O)OH or methyl.
(15-7)
   R⁸ and R⁹ are each independently, for each instance, —H, —C₁₋₆ alkyl, —C₂₋₆ alkenyl, —C₂₋₆ alkynyl, —C₃₋₈ cycloalkyl, —C₄₋₈ cycloalkenyl, heterocyclyl, aryl, or heteroaryl, wherein each of alkyl, alkenyl, alkynyl, cycloalkyl, cycloalkenyl, heterocyclyl, aryl, and heteroaryl is optionally substituted with one or more R¹⁰ or R¹¹; or
   R⁸ and R⁹, attached to an atom to which they are both attached, may form —C₃₋₈ cycloalkyl, —C₄₋₈ cycloalkenyl, spirocycloalkyl, spiroheterocyclyl, heterocyclyl, heteroaryl, or aryl, and the formed —C₃₋₈ cycloalkyl, —C₄₋₈ cycloalkenyl, spirocycloalkyl, spiroheterocyclyl, heterocyclyl, heteroaryl, or aryl is optionally substituted with one or more R¹⁰ or R¹¹;
(15-8)
   R^{8′} and R^{9′} are each independently, for each instance, —C₁₋₆ alkyl, —C₂₋₆ alkenyl, —C₂₋₆ alkynyl, —C₃₋₈ cycloalkyl, —C₄₋₈ cycloalkenyl, heterocyclyl, aryl, or heteroaryl, wherein each of alkyl, alkenyl, alkynyl, cycloalkyl, cycloalkenyl, heterocyclyl, aryl, and heteroaryl is optionally substituted with one or more R¹⁰ or R¹; or
   R⁸ and R⁹¹, attached to an atom to which they are both attached, may form —C₃₋₈ cycloalkyl, —C₄₋₈ cycloalkenyl, spirocycloalkyl, spiroheterocyclyl, heterocyclyl, heteroaryl, or aryl, and —C₃₋₈ cycloalkyl, —C₄₋₈ cycloalkenyl, spirocycloalkyl, spiroheterocyclyl, heterocyclyl, heteroaryl, or aryl is optionally substituted with one or more R¹⁰ or R¹¹;
(15-9)
   R¹⁰ and R¹¹ are each independently, for each instance, hydrogen, —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, —OH, halogen, oxo, —NO₂, —CN, —NH₂, —OC₁₋₆ alkyl, —NHC₁₋₆ alkyl, —N(C₁₋₆ alkyl)₂, —S(O)₂NH(C₁₋₆ alkyl), —S(O)₂N(C₁₋₆ alkyl)₂, —S(O)₂C₁₋₆ alkyl, —C(O) C₁₋₆ alkyl, —C(O)NH₂, —C(O)NH(C₁₋₆ alkyl), —C(O)N(C₁₋₆ alkyl)₂, —C(O) OC₁₋₆ alkyl, —N(C₁₋₆ alkyl) SO₂C₁₋₆ alkyl, —S(O)(C₁₋₆ alkyl), —S(O)N(C₁₋₆ alkyl)₂, or N(C₁₋₆ alkyl)S(O)(C₁₋₆ alkyl), wherein each of alkyl, alkenyl, alkynyl, cycloalkyl, cycloalkenyl, heterocyclyl, heteroaryl, and aryl is optionally substituted with one or more R¹²;
   wherein any two R¹⁰ or any two R¹¹, when on non-adjacent atoms, can attach and form crosslinked cycloalkyl or heterocyclyl;
   wherein any two R¹⁰ or any two R¹¹, when on adjacent atoms, can attach and form cycloalkyl, heterocyclyl, aryl, or heteroaryl;
(15-9-1)
   R¹⁰ is methoxy or fluoro.
(15-10)
   R¹² is each independently, for each instance, —H, —C₁₋₆ alkyl, —C₂₋₆ alkenyl, —C₁₋₆ alkynyl, —C₃₋₈ cycloalkyl, —C₄₋₈ cycloalkenyl, heterocyclyl, heteroaryl, aryl, —OH, halogen, oxo, —NO₂, —CN, —NH—, —OC₁₋₆ alkyl, —NHC₁₋₆ alkyl, —N(C₁₋₆ alkyl)₂, —S(O)₂NH(C₁₋₆ alkyl), —S(O)₂N(C₁₋₆ alkyl)₂, —S(O)₂C₁₋₆ alkyl, —C(O)C₁₋₆ alkyl, —C(O)NH₂, —C(O)NH(C₁₋₆ alkyl), —C(O)N(C₁₋₆ alkyl)₂, —C(O) OC₁₋₆ alkyl, —N(C₁₋₆ alkyl) SO₂C₁₋₆ alkyl, —S(O)(C₁₋₆ alkyl), —S(O)N(C₁₋₆ alkyl)₂, or N(C₁₋₆ alkyl)S(O)(C₁₋₆ alkyl).
(15-11)
   n is an integer from 1 to 4.

In formula (15), variables can be 15-1-1, 15-2-1, 15-3-1, 15-4-1, 15-6-1, and 15-9-1.

While the preferred variables in the compound of the present disclosure represented by formula (16) are described below, the technical scope of the present disclosure is not limited to the scope of the compounds described below.

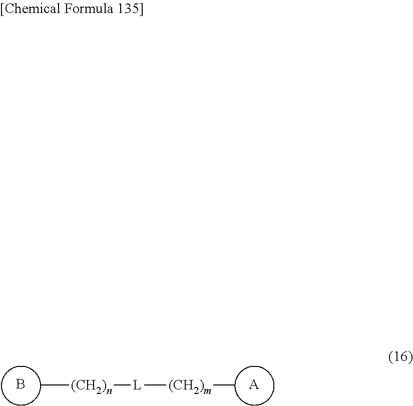

(16-1)
   ring B is a group having the following structure;

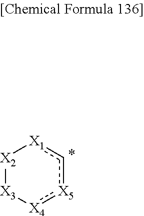

one of ring atoms X₂ and X₃ is N(R^{Kl}), and the other one of the ring atoms X₂ and X₃ is C(═O);
ring atom X₁ is selected from N(R^{X1}), C(R^{X2}), and C(═O), and ring atoms X₄ and X₅ are each independently selected from N(R^{X1}), C(R^{X3}), and C(═O); wherein at least one of the ring atoms X₁, X₄, and X₅ is different from N(R^{X1}) and C(═O); and wherein if X₃ and X₅ are C(═O), X₄ is N(R^{X1}), and X₁ is C(R^{X2}), X₂ is N(H);
each

[symbol]

[Chemical Formula 137]
is independently a single bond or a double bond; wherein at least one of any two adjacent bonds

[symbol]

[Chemical Formula 138]
is a single bond;
   each R^{X1} is independently selected from hydrogen, C₁₋₅ alkyl, —CO(C₁₋₅ alkyl), —(C₀₋₃ alkylene)-aryl, and heteroaryl,
   wherein aryl in the —(C₀₋₃ alkylene)-aryl and the heteroaryl are each optionally substituted with one or more groups R^{X11};
   R^{X2} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
   two groups R^{X3} are linked to each other and, together with the ring carbon to which they are attached, form a 5- or 6-membered cyclyl group optionally substituted with one or more groups R^{X31}, or two groups R^{X3} are each independently selected from hydrogen, C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl) (C₁₋₅ alkyl), halogen, C₁₋₅ haloalkyl, —O—(C₁₋₅ haloalkyl), —CF₃, —CN, —NO₂, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl) (C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl) (C₁₋₅ alkyl), —NH—SO₂—(C₁₋₅ alkyl), and —N(C₁₋₅ alkyl)-SO₂—(C₁₋₅ alkyl);
   each R^{X11} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)—NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl)-(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
   each R^{X31} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—N(C₁₋₅ alkyl), —(C₀₋₃ alkylene)-SO₂—N(C₁₋₅ alkyl) (C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
   ring B is attached to the remaining portion of the compound of formula (16) via a ring carbon atom marked with an asterisk (*), or if X₄ and X₅ are each C(R^{X3}) and two groups R^{X3} are linked to each other and, together with the ring carbon atom to which they are attach, form a 5- or 6-membered cyclyl group optionally substituted with one or more groups R^{X31}, ring B may be attached to the remaining portion of the compound of formula (16) via any carbocyclic ring atom of the 5- or 6-membered cyclyl group;
(16-1-1)
   ring B is

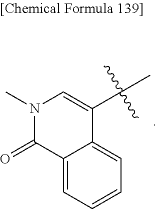 [SMILES: *c1cn(C)c(=O)c2ccccc12]

(16-2)
   ring A is aryl or heteroaryl, wherein the aryl and the heteroaryl are optionally substituted with one or more groups R^{A}, wherein the heteroaryl is selected from 1,4-benzodioxanyl, benzoxanyl, 1,3-benzodioxolanyl, benzoxolanyl, and 1,5-benzodioxepanyl;
   each R^{A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl), —(C₀₋₃ alkylene)-NH—SO—(C₁₋₅ alkyl), —(C₀₋₃ alkylene)-N(C₁₋₅ alkyl)-SO₂—(C₁₋₅ alkyl), —(C₀₋₃ alkylene)-cycloalkyl, —(C₀₋₃ alkylene)-O-cycloalkyl, —(C₀₋₃ alkylene)-O(C₁₋₅ alkylene)-cycloalkyl, —(C₀₋₃ alkylene)-heterocycloalkyl, —(C₀₋₃ alkylene)-O-heterocycloalkyl, and —(C₀₋₃ alkylene)-O(C₁₋₅ alkylene)-heterocycloalkyl;
(16-2-1)
   ring A is phenyl, 1,4-benzodioxanyl, 1,5-benzodioxepanyl, or benzooxanyl, wherein the group is optionally substituted with one or more group R^{A}, and group R^{A} is methoxy or 2-oxolanylmethyloxy.
(16-3)
   L is selected from —CO—N(R^{L1})—, —N(R^{L1})—CO—, —CO—O—, —O—CO—, —C(═N—R^{L2})—N(R^{L1})—, —N(R^{L1})—C(═N—R^{L2})—, —C(═S)—N(R^{L1})—, —N(R^{L1})—C(═S)—, —N(R^{L1})—CO—N(R^{L1})—, —O—CO—N(R^{L1})—, —N(R^{L1})—CO—O—, —N(R^{L1})—C(═N—R^{L2})—N(R^{L1}), —O—C(═N—R^{L2})—N(R^{L1})—, —N(R^{L1})—C(═N—R^{L2})—O—, —S—C(═N—R^{L2})—N(R^{L1})—, —N(R^{L1})—C(═N—R^{L2})—S—, —N(R^{L1})—C(═S)—N(R^{L1}), —O—C(═S)—N(R^{L1}), —N(R^{L1})—C(═S)—O—, —S—CO—N(R^{L1})—, and —N(R^{L1})—CO—O—;
   each R^{L1} is independently selected from hydrogen and C₁₋₅ alkyl; and
   each R^{L2} is independently selected from hydrogen, C₁₋₅ alkyl, —CN, and —N02;
(16-3-1) L is —C(O)—NH—.
(16-4)
   n is 0 or 1;
   m is 0 or 1;
(16-4-1) n and m are 0.

In formula (16), variables can be 16-1-1, 16-2-1, and 16-3-1.

While the preferred variables in the compound of the present disclosure represented by formula (17) are described below, the technical scope of the present disclosure is not limited to the scope of the compounds described below.

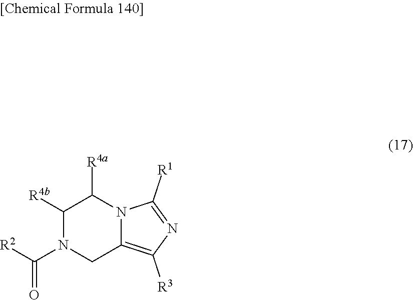

(17-1)
   R¹ is hydrogen, or selected from alkyl, amino, alkoxy, heteroalkyl, cycloalkyl, heterocycloalkyl, halogen, haloalkyl, sulfonylalkyl, aryl, and heteroaryl, which is optionally substituted with 1, 2, or 3 group R⁵;
(17-1-1)
   R¹ is cyclopropyl, 4-oxanyl, or 4,4-fluorocyclohexanyl.
(17-2)
   R² is hydrogen, or selected from alkyl, haloalkyl, amino, alkoxy, cycloalkyl, and heterocycloalkyl, which is optionally substituted with 1 or 2 group R⁶;
(17-2-1)
   R² is methylamino.
(17-3)
   R³ is selected from alkyl, amino, alkoxy, heteroalkyl, cycloalkyl, heterocycloalkyl, carbonyl, sulfonyl, aryl, and heteroaryl, wherein the R³ is:
(a) optionally substituted with 1, 2, or 3 group R⁷, and
(b) optionally substituted with one R⁸;
(22-3-1)
   R³ is isoquinoline, and (a) is optionally substituted with one R⁷, and (b) substituted with one R⁸.
(17-4)
   R^{4a} and R^{4b} are hydrogen;
(17-5)
   R⁵, R⁶, and R⁷ are each independently selected from alkyl, alkoxy, cyano, carboxy, halogen, haloalkyl, haloalkoxyl, hydroxy, and oxo;
(17-5-1)
   R⁷ is selected from difluoromethyl, trifluoromethyl, and a fluorine atom.
(17-6)
   R⁸ is selected from aryl, heteroaryl, and heterocycloalkyl, wherein the R⁸ is optionally substituted with 1, 2, or 3 group R¹⁰;
(17-6-1)
   R⁸ is selected from pyridyl, thiazolyl, and pyrazolyl,
   wherein the group is optionally substituted with one group R¹⁰.
(17-6)
   R¹⁰ is each independently selected from alkyl, cycloalkyl, (cycloalkyl)alkyl, heterocycloalkyl, (heterocycloalkyl)alkyl, aryl, (aryl)alkyl, (heteroaryl)alkyl, alkoxy, cyano, carboxy, halogen, haloalkyl, haloalkoxy, hydroxy, hydroxyalkyl, oxo, CONH₂, CONHCH₃, SO₂CH₃, and SO₂NH₂;
(17-6-1)
   R¹⁰ is selected from —C(O)NHCH₃ and methyl.

In formula (17), variables can be 17-1-1, 17-2-1, 17-3-1, 17-5-1, and 17-6-1.

While the preferred variables in the compound of the present disclosure represented by formula (18) are described below, the technical scope of the present disclosure is not limited to the scope of the compounds described below.

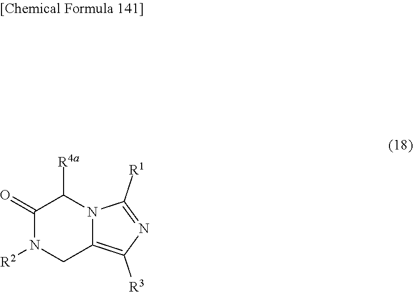

(18-1)
   R¹ is hydrogen, or selected from alkyl, amino, alkoxy, heteroalkyl, cycloalkyl, heterocycloalkyl, halogen, haloalkyl, sulfonylalkyl, aryl, and heteroaryl, which is optionally substituted with 1, 2 or 3 group R⁵;
(18-1-1)
   R¹ is cyclopropyl, 4-oxanyl, or 4-methyl-4-oxanyl.
(18-2)
   R² is hydrogen, or selected from alkyl, heteroalkyl, cycloalkyl, heterocycloalkyl, and haloalkyl, which is optionally substituted with 1, 2, or 3 group R⁶;
(18-2-1)
   R² is methyl.
(18-3)
   R³ is selected from alkyl, amino, alkoxy, heteroalkyl, cycloalkyl, heterocycloalkyl, alkylcarbonyl, alkylsulfonyl, arylcarbonyl, arylsulfonyl, aryl, and heteroaryl, wherein the R³ is: (a) optionally substituted with 1, 2, or 3 group R⁷, and (b) optionally substituted with one R⁸;
(18-3-1)
   R³ is isoquinoline, and (a) is optionally substituted with one R⁷, and (b) substituted with one R⁸.
(18-4)
   R^{4a} is selected from hydrogen, halogen, alkyl, heteroalkyl, cycloalkyl, and heterocycloalkyl, wherein the R^{4a} is optionally substituted with 1, 2 or 3 group R⁹;
(18-4-1)
   R^{4a} is methyl.
(18-5)
   R⁵ is each independently selected from alkyl, alkoxy, alkoxyalkyl, alkylcarbonyl, alkylsulfonyl, amino, aminocarbonyl, cyano, carboxy, halogen, haloalkoxy, haloalkyl, hydroxy, hydroxyalkyl, and oxo;
(18-6)
   R⁶ and R⁷ are each independently selected from alkyl, alkoxy, cyano, carboxy, halogen, haloalkyl, hydroxy, and oxo;
(18-6-1)
   R⁷ is selected from difluoromethyl, trifluoromethyl, and cyano.
(18-7)
   R⁸ is selected from heterocycloalkyl, aryl, and heteroaryl, wherein the R⁸ is optionally substituted with 1, 2, or 3 group R¹⁰;
(18-7-1)
   R⁸ is selected from thiazolyl, tetrazolyl, thiadiazole, oxanyl, oxenyl, pyrazolyl, imidazolyl, and oxadiazolyl,
   wherein the group is optionally substituted with 1 or 2 group R¹⁰.
(18-8)
   R⁹ is each independently selected from alkyl, alkoxy, cyano, carboxy, halogen, haloalkyl, hydroxy, and oxo.
(18-9)
   R¹⁰ is each independently selected from alkyl, alkoxy, cyano, carboxy, halogen, haloalkyl, and hydroxy;
(18-9-1)
   R¹⁰ is selected from methyl and oxo.

In formula (18), variables can be 18-1-1, 18-2-1, 18-3-1, 18-4-1, 18-6-1, 18-7-1, and 18-9-1.

While the preferred variables in the compound of the present disclosure represented by formula (19) are described below, the technical scope of the present disclosure is not limited to the scope of the compounds described below.

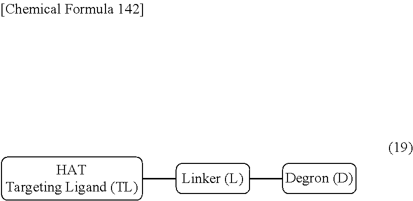

(19-1)
Targeting Ligand (TL) represents a structure that attaches to P300.
(19-1-1)
Targeting Ligand (TL) is represented by

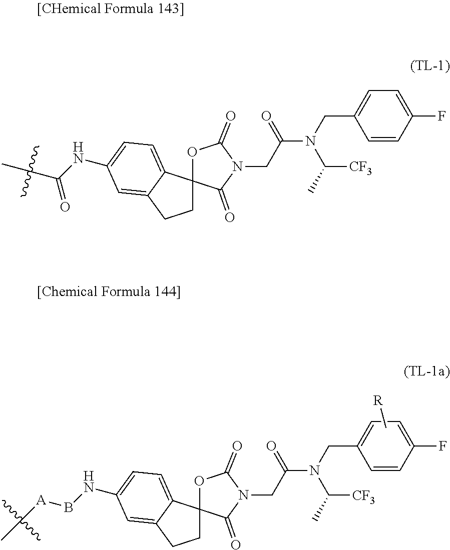

(wherein A is CH₂, NH, or O, B is CH₂ or CO, and R is hydrogen, halogen, CN, CF₃, alkyl, or alkoxy)

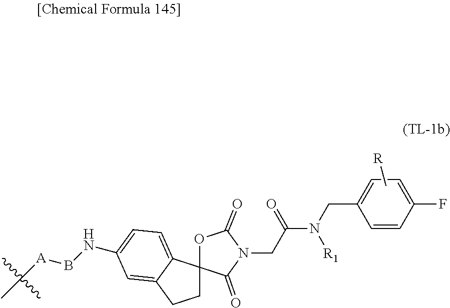

(wherein R¹ is a C3-C5 carbocyclic ring group, an alkylcarbocyclic ring group, a 3- to 5-membered N-heterocyclic group, or an alkyl N heterocyclic group, and an alkyl group is a C1-C10 alkyl group)

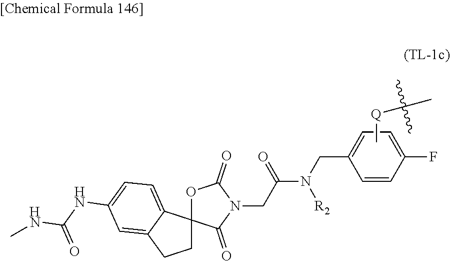

(wherein Q is CH₂, O, N, CO, C(O)O, C(O)N, CH₂N, CH₂C(O), CH₂C(O)N, or CH₂CH₂N, R₂ is

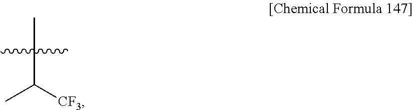 [SMILES: *C(C)C(F)(F)F]

a C3-C5 carbocyclic ring group, an alkylcarbocyclic ring group, a 3- to 5-membered N-heterocyclic group, or an alkyl N heterocyclic group, and an alkyl group is a C1-C10 alkyl group)

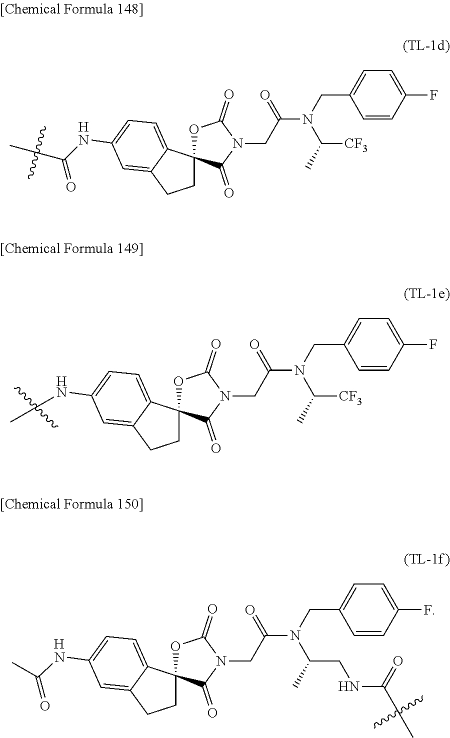

(19-2)
Linker represents a structure that covalently attaches to Degron and Targeting Ligand.
(19-2-1)
Linker is represented by

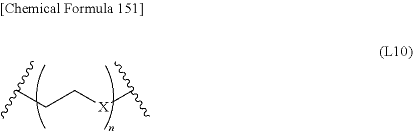

(wherein X is CH₂, NH, NMe, or O, and n is an integer from 0 to 11).
(19-2-2)
Linker is represented by

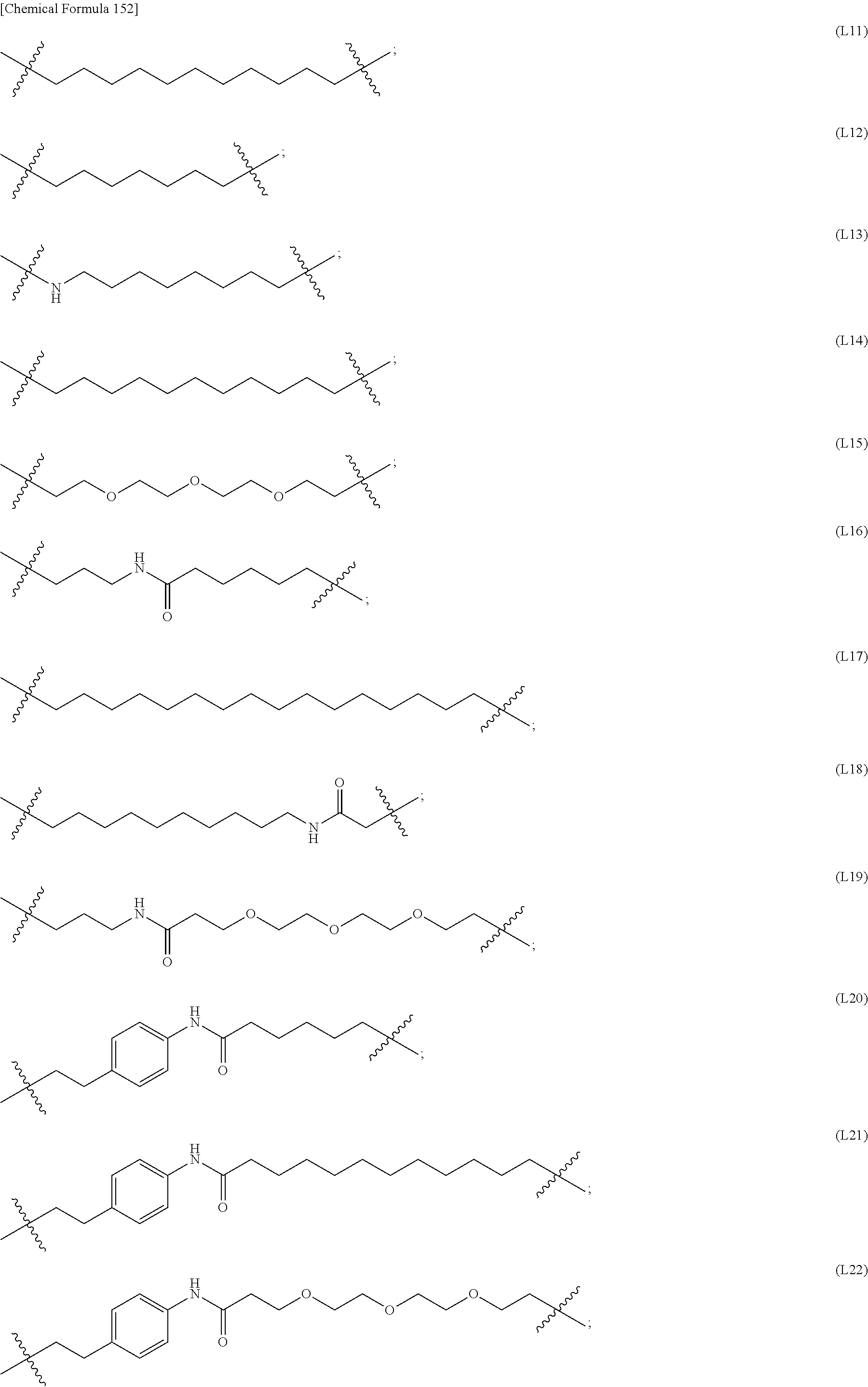

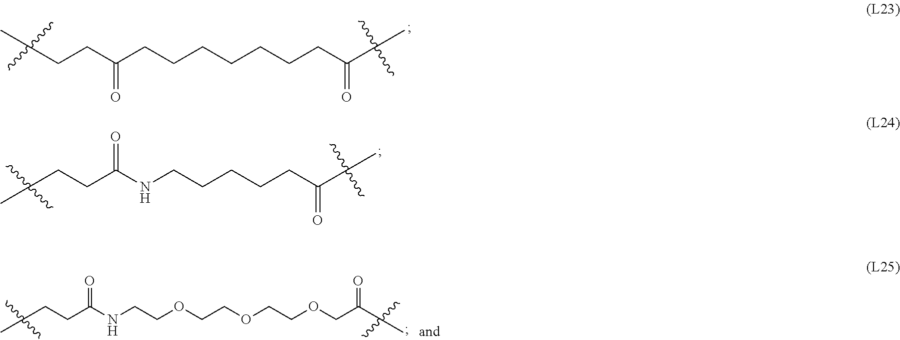

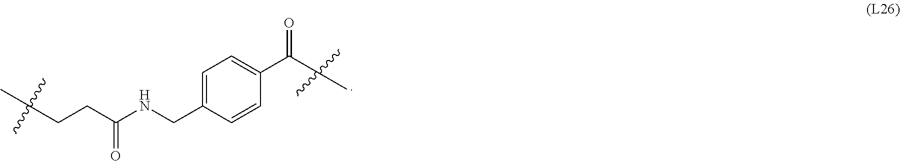 [SMILES: *CCC(=O)NCc1ccc(C(*)=O)cc1]

(19-3)
Degron represents a structure that attaches to an E3 ubiquitin ligase.
(19-3-1)
Degron is represented by

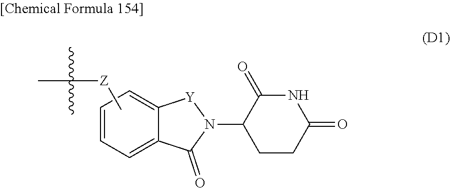

(wherein Y is CH₂ or CO, Z is NH, O, or OCH₂CO, and a wavy line represents a point of attachment to Linker)

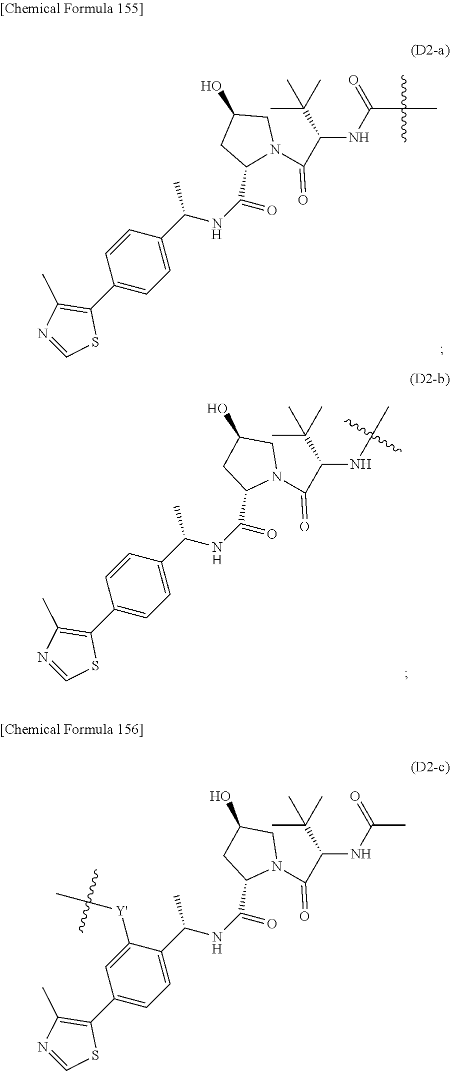

(wherein Y′ is a bond, N, O, or C)

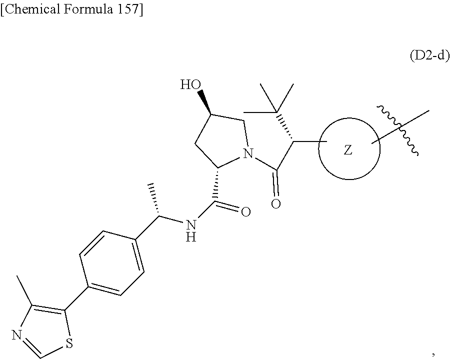

(wherein Z is a C5-C6 carbocyclic ring group or a C5-C6 heterocyclic group)

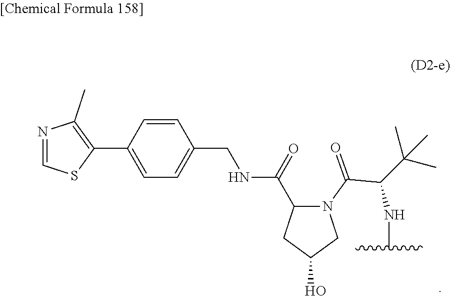 [SMILES: *N[C@H](C(=O)N1C[C@H](O)CC1C(=O)NCc1ccc(-c2scnc2C)cc1)C(C)(C)C]

In formula (19), variables can be 19-1-1, 19-2-1, and 19-3-1.

While the preferred variables in the compound of the present disclosure represented by formula (20) are described below, the technical scope of the present disclosure is not limited to the scope of the compounds described below.

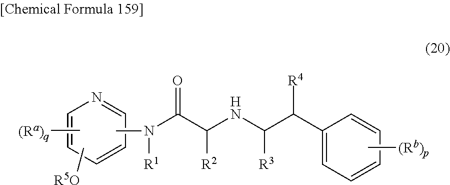

(20-1)
   R¹, R³, and R⁴ are each independently hydrogen or C₁₋₄ alkyl.
(20-1-1)
   R¹ and R³ are hydrogen, and R⁴ is hydrogen or methyl.
(20-2)
   R² is phenyl or 5- to 6-membered heteroaryl, each optionally substituted with 1 to 3 R^{C}.
(20-2-1)
   R² is phenyl or pyrazolyl, each optionally substituted with 1 to 3 groups selected from R^{C}, wherein R^{c} is halogen, C₁₋₆ alkyl, halo(C₁₋₆ alkyl), C₁₋₆ alkoxy, and halo(C₁₋₆ alkoxy).
(20-3)
   R⁵ is C₁₋₆ alkyl substituted with 4- to 6-membered heterocyclyl or 5- to 6-membered heteroaryl (wherein the heterocyclyl and heteroaryl are optionally substituted with 1 to 3 R^{d}), 4- to 6-membered heterocyclyl (wherein the heterocyclyl is optionally substituted with 1 to 3 R^{d}), or 5 to 6-membered heteroaryl (wherein the heteroaryl is optionally substituted with 1 to 3 R^{d}).
(20-3-1)
   R⁵ is C₁₋₄ alkyl substituted with pyrazolyl or oxazolidinyl, each optionally substituted with 1 to 3 groups selected from R^{d}, or R⁵ is piperidinyl, azetidinyl, hexahydropyrimidinyl, tetrahydrofuranyl, tetrahydropyranyl, oxetanyl, pyrazolyl, pyrrolidinyl, or pyrimidinyl, each optionally substituted with 1 to 3 groups selected from R^{d}, and R^{d} is selected from halogen, oxo, C₁₋₄ alkyl, C₁₋₄ alkoxy, halo(C₁₋₄ alkyl), —C₁₋₄ alkyl OR^{e}, —C(O)R^{f}, —C(O)N(R^{e})₂, —C₁₋₆ alkyl C(O)N(R^{e})₂, and —S(O)₂R^{e}.
(20-4)
R^{a}, R^{b}, R^{c}, and R^{d} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{e}, —C(O)R^{f}, —C(O)OR, —C₁₋₆ alkyl C(O)OR^{e}, —C(O)N(R^{e})₂, —C(O)NR^{e}C₁₋₆ alkyl OR^{e}, —OC₁₋₆ alkyl N(R^{e}), —C₁₋₆ alkyl C(O)N(R^{e}), —C₁₋₆ alkyl N(R^{e}), —N(R^{e})₂, —C(O)NR^{e}C₁₋₆ alkyl N(R^{e})₂, —NR^{e}C₁₋₆ alkyl N(R^{e})₂, —NR^{e}C₁₋₆ alkyl OR^{e}, —SOR^{e}, —S(O)₂R^{e}, —SON(R^{e})₂, —SO₂N(R^{e}), —O(C₃₋₆) cycloalkyl, —O—C₁₋₄ alkyl-aryl, —C₁₋₆ alkyl(C₃₋₆) cycloalkyl, —C₁₋₆ alkylaryl, —C₁₋₆ alkylheteroaryl, —C₁₋₆ alkylheterocyclyl, C₃₋₆ cycloalkyl, heterocyclyl, heteroaryl, or aryl (wherein each of them, alone or attached to —O(C₃₋₆)cycloalkyl, —C₁₋₆ alkyl(C₃₋₆) cycloalkyl, —C₁₋₆ alkylaryl, —C₁₋₆ alkylheteroaryl, or —C₁₋₆ alkylheterocyclyl, is optionally substituted with 1 to 3 groups selected from halogen, C₁₋₆ alkyl, C₁₋₆ haloalkyl, C₁₋₆ alkoxy, C₁₋₆ haloalkoxy, —N(R^{e})₂, —C(O)R^{f}, and —C₁₋₆ alkyl OR^{e}).
(20-4-1)
   R^{a} is C₁₋₃ alkyl, C₁₋₃ alkoxy, halo(C₁₋₃ alkyl), halo C₁₋₃ alkoxy, or halogen,
   R^{b} is halogen, cyano, or —SO₂NH₂,
   R^{c} is halogen, C₁₋₆ alkyl, halo(C₁₋₆ alkyl), C₁₋₆ alkoxy, or halo(C₁₋₆ alkoxy),
   R^{d} is selected from halogen, oxo, C₁₋₄ alkyl, C₁₋₄ alkoxy, halo(C₁₋₄ alkyl), —C₁₋₄ alkyl, OR^{e}, —C(O)R^{f}, —C(O)N(R^{e})₂, —C₁₋₆ alkyl C(O)M(R^{c})₂, and —S(O)₂R^{c}.
(20-4)
   each R^{c} is hydrogen, C₁₋₄ haloalkyl, or C₁₋₄ alkyl,
   each R^{f} is hydrogen, C₁₋₄ haloalkyl, C₁₋₄ alkyl, or C₃₋₄ cycloalkyl,
(20-4-1)
   R^{c} is hydrogen or C₁₋₃ alkyl, and
   R^{f} is hydrogen or C₁₋₄ alkyl.
(20-5)
   q is 0, 1, or 2, and
   p is 0, 1, 2, or 3,

In formula (20), variables can be 20-1-1, 20-2-1, 20-3-1, 20-4-1, and 20-5.

While the preferred variables in the compound of the present disclosure represented by formula (21) are described below, the technical scope of the present disclosure is not limited to the scope of the compounds described below.

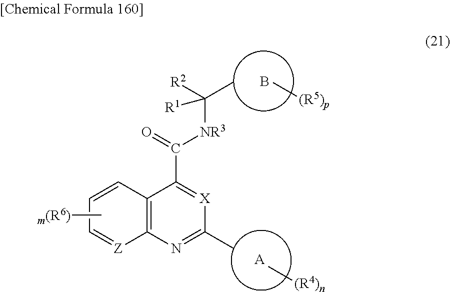

(21-1)
   X is CH or N;
   Z is N, CH, or CR⁶.
(21-1-1)
   X is N, and Z is N, or only one of X and Z is N.
(21-2)
   ring A is monocyclic aryl, bicyclic aryl, monocyclic heterocyclyl, or bicyclic heterocyclyl.
(21-2-1)
   ring A is phenyl, 5- or 6-membered heteroaryl, 9- or 10-membered bicyclic heteroaryl, 5- to 7-membered saturated monocyclic heterocyclyl, or 9- to 10-membered bicyclic non-aromatic heterocyclyl.
(21-3)
   ring B is 5-membered N-containing heteroaryl.
(21-3-1)
   ring B is pyrrole, pyrazole, imidazole, oxazole, isoxazole, thiazole, or isothiazole.
(21-4)
   R¹ and R² are each independently hydrogen, C₁₋₆ alkyl, a halogen atom, CN, —C(O)R^{1a}, —C(O)OR^{1a}, —C(O)N(R^{1a})₂, —N(R^{1a}), —N(R^{1a})C(O)R^{1a}, —N(R^{1a})C(O)OR^{1a}, —N(R^{1a})C(O)N(R^{1a})₂, —N(R^{1a})S(O)OR^{1a}, —OR^{1a}, —OC(O)R^{1a}, —OC(O)N(R^{1a})₂, —SR^{1a}, —S(O)R^{1a}, —S(O)₂R^{1a}, —S(O)N(R^{1a})₂, or —S(O)₂N(R^{1a})₂;
   R^{1a} is each independently hydrogen, C₁₋₆ alkyl, C₂₋₆ alkenyl, C₂₋₆ alkynyl, carbocyclyl, or heterocyclyl, or two R^{1a}, together with the nitrogen atom to which they are attached, may form a 4- to 7-membered ring (wherein the 4- to 7-membered ring may contain 1 to 2 each independently selected nitrogen atoms, oxygen atoms, or sulfur atoms).
(21-4-1)
   R¹ and R² are each independently selected from hydrogen, C₁₋₆ alkyl, and halogen.
(21-5)
   R³ is hydrogen or C₁₋₆ alkyl.
(21-5-1)
   R³ is hydrogen, or C₁₋₆ alkyl optionally substituted with halo, —OR⁷, or —N(R⁷)₂, and R⁷ is hydrogen or C₁₋₃ alkyl.
(21-6)
   R⁴ is each independently C₁₋₆ alkyl, C₂₋₆ alkenyl, C₂₋₆ alkynyl, carbocyclyl, heterocyclyl, a halogen atom, CN, —C(O)R^{4a}, —C(O)OR^{4a}, —C(O)N(R^{4a}), —N(R^{4a}), —N(R^{4a})C(O)R^{4a}, —N(R^{4a})C(O)OR^{4a}, —N(R^{4a})C(O)N(R^{4a})₂, —N(R^{4a})S(O)OR^{4a}, —OR^{4a}, —OC(O)R^{4a}, —OC(O)N(R^{4a}), —SR^{4a}, —S(O)R^{4a}, —S(O)₂R^{4a}, —S(O)N(R^{4a})₂, —S(O)₂N(R^{4a})₂, or —P(O)(R^{4a})₂;
   R^{4a} is each independently hydrogen, C₁₋₆ alkyl, C₂₋₆ alkenyl, C₂₋₆ alkynyl, carbocyclyl, heterocyclyl, or —P(O)(R^{7a})₂, or two R^{4a}, together with the nitrogen atom to which they are attached, may form a 4- to 7-membered ring (wherein the group may contain 1 to 2 each independently selected nitrogen atoms, oxygen atoms, or sulfur atoms.
(21-6-1)
   R⁴ is each independently selected from C₁₋₆ alkyl, C₃₋₆ cycloalkyl, 5- to 6-membered heterocyclyl, halogen, —CN, —C(O)R^{4a}, —C(O)₂R^{4a}, —C(O)N(R^{4a})₂, —N(R^{4a})₂, —N(R^{4a}) C(O) R^{4a}, —N(R^{4a}) C(O)₂R^{4a}, —N(R^{4a}) C(O)N(R^{4a})₂, —N(R^{a}) S(O)₂R^{4a}, —OR^{4a}, —OC(O) R^{4a}, —OC(O)N(R^{4a})₂, and —S(O)₂R^{4a},
   R^{4a} is each independently hydrogen, C₁₋₆ alkyl, C₃₋₆ cycloalkyl, or 5- to 6-membered heterocyclyl.
(21-7)
   R⁵ is each independently C₁₋₆ alkyl or carbocyclyl, or two R⁵, together with the atom to which they are attached, may form a 4- to 7-membered ring (wherein the 4- to 7-membered ring may contain 1 to 2 each independently selected nitrogen atoms, oxygen atoms, or sulfur atoms).
(21-7-1)
   R⁵ is each independently selected from C₁₋₄ alkyl and C₃₋₆ cycloalkyl, each optionally substituted with 1 to 3 halogen.
(21-8)
   R⁶ is each independently C₁₋₆ alkyl, C₂₋₆ alkenyl, C₂₋₆ alkynyl, carbocyclyl, heterocyclyl, a halogen atom, —CN, —C(O) R^{6a}, —C(O)OR^{6a}, —C(O)N(R^{6a}), —N(R^{6a}), —N(R^{6a}) C(O) R^{6a}, —N(R^{6a}) C(O) OR^{6a}, —N(R^{6a}) C(O)N(R^{6a})₂, —N(R^{6a}) S(O) OR^{6a}, —OR^{6a}, —OC(O)R^{6a}, —OC(O)N(R^{6a})₂, —SR^{6a}, —S(O)R^{6a}, —S(O)₂R^{6a}, —S(O)N(R^{6a})₂, —S(O)₂N(R^{6a})₂, or —P(O)(R^{6a})₂;
   R^{6a} is each independently hydrogen, C₁₋₆ alkyl, C₂₋₆ alkenyl, C₂₋₆ alkynyl, carbocyclyl, or heterocyclyl, or two R^{6a}, together with the nitrogen atom to which they are attached, may form a 4- to 7-membered ring (wherein the 4- to 7-membered ring may contain 1 to 2 each independently selected nitrogen atoms, oxygen atoms, or sulfur atoms).
(21-8-1)
   R⁶ is C₁, Br, F, —CN, —OCH₃, —CH₃, —CH₂CH₃, —OCH₂CH₃, —NH₂, —NHCH₃, —N(CH₃)₂, —C₂H₄NHCH₃, —OCH₂CH(OH) CH₂NHCH₃, morpholine, or —CH₂OCH₃.
(21-9)
   m is 0, 1, 2, or 3;
   p is 0, 1, 2, or 3;
   n is 0, 1, 2, 3, 4, 5, or 6;
   the C₁₋₆ alkyl, C₂₋₆ alkenyl, C₂₋₆ alkynyl, carbocyclyl, and heterocyclyl is optionally substituted with one or two or more independent R⁷, halogen atom, —CN, —C(O)R⁷, —C(O)OR⁷, —C(O)N(R⁷)₂, —N(R⁷)₂, —N(R⁷)C(O)R⁷, —N(R⁷)C(O)OR⁷, —N(R⁷)C(O)N(R⁷)₂, —N(R⁷) S(O) OR⁷, —OR⁷, —OC(O) R⁷, —OC(O)N(R⁷)₂, —SR⁷, —S(O)R⁷, —S(O)₂R⁷, —S(O)N(R⁷)₂, —S(O)₂N(R⁷)₂, or —P(O)(R⁷)₂;
(21-10)
   R⁷ is each independently hydrogen, C₁₋₆ alkyl, C₂₋₆ alkenyl, C₂₋₆ alkynyl, carbocyclyl, or heterocyclyl, wherein the C₁₋₆ alkyl, the C₂₋₆ alkenyl, the C₂₋₆ alkynyl, the carbocyclyl, or the heterocyclyl is optionally substituted with one or two or more substituents selected from R^{7a}, a halogen atom, —CN, —C(O)R^{7a}, —C(O)OR^{7a}, —C(O)N(R^{7a})₂, —N(R^{7a})₂, —N(R^{7a}) C(O) R^{7a}, —N(R^{7a}) C(O) OR^{7a}, —N(R^{7a})C(O)N(R^{7a})₂, —N(R^{7a}) S(O) OR^{7a}, —OR^{7a}, —OC(O) R^{7a}, —OC(O)N(R^{7a})₂, —SR^{7a}, —S(O)R^{7a}, —S(O)₂R^{7a}, —S(O)N(R^{7a})₂, —S(O)₂N(R^{7a})₂, and —P(O)(R^{7a})₂;
   R^{7a} is each independently hydrogen or C₁₋₄ alkyl.
(21-10-1)
   R⁷ is hydrogen or C₁₋₃ alkyl.

In formula (21), variables can be 21-1-1, 21-2-1, 21-3-1, 21-4-1, 21-5-1, 21-6-1, 21-7-1, 21-8-1, 21-9, and 21-10-1.

While the preferred variables in the compound of the present disclosure represented by formula (22) are described below, the technical scope of the present disclosure is not limited to the scope of the compounds described below.

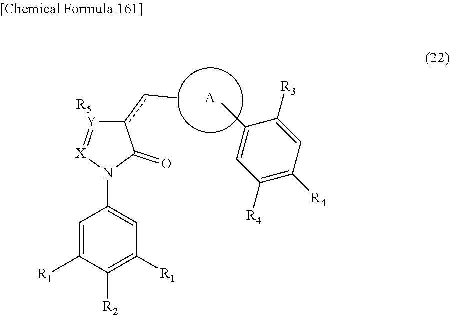

(22-1)
   ring A is 5- or 6-membered aryl, or heteroaryl comprising a nitrogen, oxygen, or sulfur atom, and 1 to 4 carbons.
(22-1-1)
   ring A is

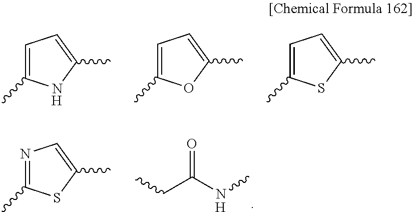

(22-2)
   R¹ is hydrogen or halogen;
   R² is a hydroxyl group, carboxyl, C₁₋₄ sulfoalkyl, boronic acid, or nitrogen-containing 5-membered heteroaryl;
   R³ is trifluoromethyl, trifluoromethoxy, phosphinyl, nitro, difluoromethyl, or cyclopentanone-containing carbocyclyl.
(22-2-1)

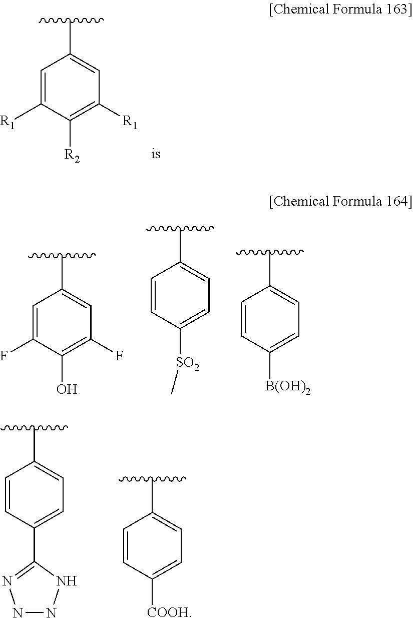

(22-3)
   R⁴ is hydrogen or methyl;
   R⁵ is hydrogen, C₁₋₄ alkyl, or cycloalkyl.
(22-3-1)

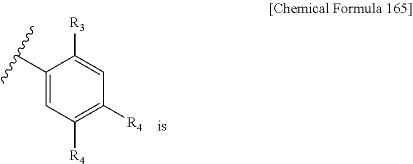

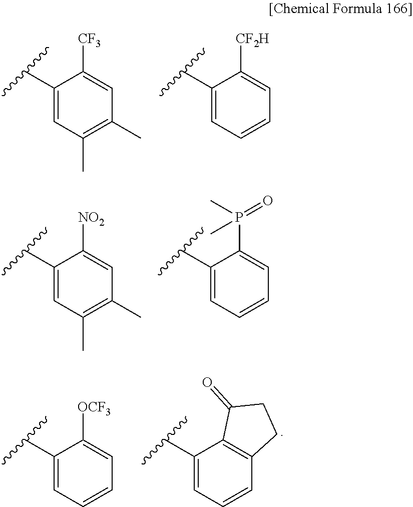

(22-4)
   X is —C(O)— or —N—;
   Y is a carbon atom, a sulfur atom, or —NH—,
   if X is —N═, Y is a carbon atom, and there is a double bond between X and Y, and if X is —C(O)—, Y is a sulfur atom or —NH—, and there is a single bond between X and Y, but an R⁵ group is absent.
(22-4-1)

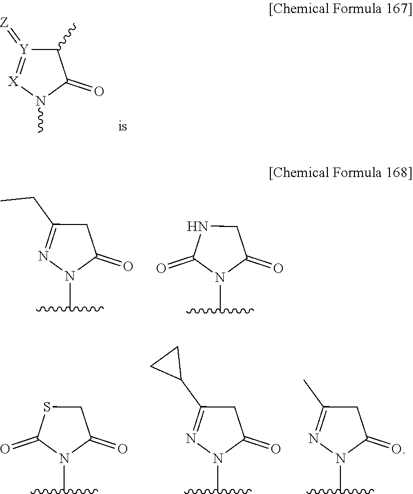

In formula (22), variables can be 22-1-1, 22-2-1, 22-3-1, and 22-4-1.

While the preferred variables in the compound of the present disclosure represented by formula (23) are described below, the technical scope of the present disclosure is not limited to the scope of the compounds described below.

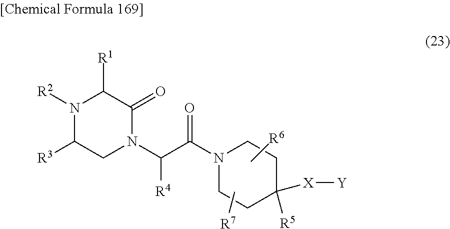

(23-1)
   R¹ is hydrogen, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with cycloalkyl, aryl, or heteroaryl (wherein the cycloalkyl, the aryl, or the heteroaryl is optionally substituted with halogen, C₁₋₄ alkyl, or C₃₋₅ cycloalkyl).
(23-1-1)
   R¹ is C₃₋₇ alkyl, C₃₋₇ cycloalkyl, or C₁₋₇ alkyl substituted with aryl or heteroaryl.
(23-2)
   R² is each independently hydrogen, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkylene-O—C₁₋₃ alkylene-O—C₁₋₃ alkylene, C₁₋₅ alkyl-NHCOR¹³, or C₁₋₃ alkyl substituted with cycloalkyl, aryl, or heteroaryl (wherein the cycloalkyl, the aryl, or the heteroaryl is optionally substituted with a halogen atom, C₁₋₄ alkyl, or C₃₋₅ cycloalkyl); with the proviso that if R² is C(O)NR¹⁵R¹⁵, both R¹⁵ may form a ring comprising a nitrogen atom of NR¹⁵R¹⁵ (wherein the ring may further comprise a heteroatom selected from an oxygen atom and a nitrogen atom, and if a nitrogen atom is contained, it is optionally substituted with R⁸).
(23-2-1)
   R² is hydrogen, C(O)R¹⁴ (wherein R¹⁴ is C₁₋₇ alkyl), C₁₋₇ alkyl, C₃₋₇ cycloalkyl, C₁₋₅ alkyl-OR⁸, C₁₋₅ alkyl-NHCOR¹³ (wherein R¹³ is pentylamino-5-oxopentyl-7-thia-2,4-diazabicyclo[3.3.0]octan-3-one), or C₁₋₃ alkyl substituted with aryl, and aryl is optionally substituted with halogen, C₁₋₄ alkyl, or C₃₋₅ cycloalkyl.
(23-3)
   R³ and R⁷ are each independently hydrogen, C₁₋₇ alkyl, C₂₋₇ alkenyl, C₂₋₇ alkynyl, C₃₋₇ cycloalkyl, or C₄₋₇ cycloalkenyl, which are optionally substituted with a halogen atom, OR⁸, NR⁸R¹¹, or C₁₋₃ alkyl substituted with aryl and heteroaryl (wherein the aryl and the heteroaryl are optionally substituted with a halogen atom, C₁₋₄ alkyl, or C₃₋₅ heteroalkyl).
(23-3-1)
   R³ and R⁷ are hydrogen.
(23-4)
   R⁴ is C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with cycloalkyl, aryl, or heteroaryl (wherein the cycloalkyl, the aryl, or the heteroaryl is optionally substituted with a halogen atom, C₁₋₄ alkyl, or C₃₋₅ heteroalkyl);
(23-4-1)
   R⁴ is C₃₋₇ alkyl, C₃₋₇ cycloalkyl, or C₁₋₃ alkyl substituted with aryl or heteroaryl.
(23-5)
   R⁵ is hydrogen, C₁₋₇ alkyl, C₂₋₇ alkenyl, C₂₋₇ alkynyl, C₃₋₇ cycloalkyl, C₄₋₇ cycloalkenyl, OR^{B}, C₁₋₃ alkyl-OR^{B}, or SR^{B}, wherein R⁵, together with X and Y, may form a ring that may comprise a carbonyl group.
(23-5-1)
   R⁵ is selected from hydrogen, C₁₋₇ alkyl, OR⁸, and SR⁸, C₁₋₇ alkyl, OR⁸, or SR⁸ of R⁵, together with X and Y, may form a ring, and the ring may comprise a carbonyl group.
(23-6)
   R⁶ is hydrogen, C₁₋₇ alkyl, C₂₋₇ alkenyl, C₂₋₇ alkynyl, C₃₋₇ cycloalkyl, C₄₋₇ cycloalkenyl (wherein the C₁₋₇ alkyl, the C₂₋₇ alkenyl, the C₂₋₇ alkynyl, the C₃₋₇ cycloalkyl, or the C₄₋₇ cycloalkenyl is optionally substituted with a halogen atom, OR⁸, NR⁸R¹¹, C₁₋₃ alkyl substituted with C(O)NR⁸R¹¹, or C₁₋₃ alkyl substituted with aryl or heteroaryl (wherein the aryl or the heteroaryl is optionally substituted with a halogen atom, C₁₋₄ alkyl, or C₃₋₅ cycloalkyl), wherein R⁶ may form a ring with any part of X, or is imidazolidinone;
(23-6-1)
   R⁶ is hydrogen, C₁₋₇ alkyl, or imidazolidinone.
(23-7)
   R⁸ and R¹¹ are each independently hydrogen, C₁₋₇ alkyl, C₂₋₇ alkenyl, C₂₋₇ alkynyl, C₃₋₇ cycloalkyl, or C₄₋₇ cycloalkenyl.
(23-7-1)
   R⁸ and R¹¹ are each independently hydrogen, C₁₋₇ alkyl, C₂₋₇ alkenyl, or C₃₋₇ cycloalkyl.
(23-8)
   X is a bond, C₁₋₇ alkylene, C₂₋₇ alkenylene, C₂₋₇ alkynylene, C₃₋₉ cycloalkylene, C₄₋₆ cycloalkenylene, —O—, C₁₋₃ alkylene-O—, —O—C₁₋₇ alkylene, —O—C₃₋₉ cycloalkylene, C₁₋₃ alkylene-O—C₁₋₇ alkylene, C₁₋₇ heteroalkylene, or —S—C₁₋₇ alkylene, wherein X, together with R⁵, R⁶, and Y, may form a polycyclic system or a ring that may comprise a carbonyl group.
(23-8-1)
   X is selected from a bond, —O—C₁₋₇ alkylene, —S—C₁₋₇ alkylene, and C₁₋₇ alkylene, and —O—C₁₋₇ alkylene, —S—C₁₋₇ alkylene, or C₁₋₇ alkylene of X, together with R⁵, may form a ring, wherein the ring may comprise a carbonyl group.
(23-9)
   Y is hydrogen, C(O)NR¹⁰R¹², C(O) OR¹⁰, R¹⁰NC(O)NR¹⁰R¹², OC(O) R¹⁰, OC(O)NR¹⁰R¹², S(O)ₙR⁸ wherein n is 0, 1, or 2, SO₂NR¹⁰R¹², NR¹⁰SO₂R¹⁰, NR¹⁰R¹², HNCOR⁸, CN, C₃₋₇ cycloalkyl that may comprise a nitrogen atom optionally substituted with R⁸ or an oxygen atom within a ring, S-aryl, O-aryl, S-heteroaryl, O-heteroaryl (wherein the S-aryl, the O-aryl, the S-heteroaryl, or the O-heteroaryl is optionally substituted with one or two or more R⁹ or R¹⁴), aryl, or heteroaryl (wherein the aryl or the heteroaryl is optionally substituted with one or two or more R⁸); wherein Y may form a ring that may comprise a carbonyl group at any position on X or R⁵, but if Y is C(O)NR¹⁰R¹² or NR¹⁰R¹², R¹⁰ and R¹² may form a ring comprising a nitrogen atom of NR¹⁰R¹² (wherein the ring may further comprise a heteroatom selected from an oxygen atom and a nitrogen atom, and if a nitrogen atom is contained, it is optionally substituted with R⁸).
(23-9-1)
   Y is selected from C(O)NR¹⁰R¹², NR¹⁰R¹², C₃₋₇-cycloalkyl that may comprise a heteroatom selected from 0 and N in a ring, wherein the heteroatom, in case of N, is optionally substituted with R⁸; S-aryl, O-aryl, S-heteroaryl, O-heteroaryl optionally substituted with one or more R⁹ or R¹⁴; and heteroaryl optionally substituted with one or more R^{A}; and Y can form with ring with any part of X or R¹, wherein the ring may comprise a carbonyl group; provided that if Y is C(O)NR¹⁰R¹² or NR¹⁰R¹², R¹⁰ and R¹² can form a ring, wherein the ring comprises N of NR¹⁰R¹², and an additional heteroatom is selected from any of O and N, wherein if the additional heteroatom is N, it is optionally substituted with R⁸.
(23-9)
   R⁹ is hydrogen, a halogen atom, C₁₋₅ alkyl, C₂₋₅ alkenyl, C₂₋₅ alkynyl, C₃₋₅ cycloalkyl, C₁₋₅ alkyl-OR^{B}, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR₈C(O)NR⁸R¹¹, OC(O)NR⁸R¹¹, SO₂NR⁸R¹¹, NR⁸SO₂R⁸, OR⁸, NR⁸R¹¹, or S(O)nR⁸ wherein n is 0, 1, or 2.
(23-9-1)
   R⁹ is selected from H, C₁₋₅ alkyl, halogen, C₁₋₅ alkyl-NR⁸R¹¹, C₁₋₅ alkyl-C(O)OR⁸, C₁₋₅ alkyl-C(O)NR⁸R¹¹, CN, C(O)R⁸, C(O)NR⁸R¹¹, C(O)OR⁸, and OR⁸.
(23-10)
   R¹⁰ and R¹² are each independently hydrogen, C₁₋₇ alkyl, C₂₋₇ alkenyl, C₂₋₇ alkynyl, C₃₋₇ cycloalkyl, or C₄₋₇ cycloalkenyl, C₁₋₃ alkylene-O—C₁₋₃ alkylene-O—C₁₋₃ alkylene, C₁₋₃ alkyl-aryl, or C₁₋₃ alkyl-heteroaryl, wherein R¹⁰ and R¹² are optionally substituted with a halogen atom, OR₈, or NR₈R¹¹.
(23-10-1)
   R¹⁰ and R¹² are each independently selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, and C₁₋₃ alkyl-heteroaryl, wherein these groups are optionally substituted with halogen or OR⁸.
(23-11)
   R¹³ is C₁₋₇ alkyl substituted with a bicycle that may comprise at least one heteroatom or carbonyl group.
(23-12)
   R¹⁴ is hydrogen, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with aryl or heteroaryl (wherein the aryl or the heteroaryl is optionally substituted with a halogen atom, C₁₋₄ alkyl, or C₃₋₅ heteroalkyl).
(23-12-1)
   R¹⁴ is C₁₋₇ alkyl.
(23-13)
   R¹⁵ is each independently hydrogen, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.
(23-13-1)
   R¹⁵ is hydrogen or C₁₋₇ alkyl.

In formula (23), the variables can be 23-1-1, 23-2-1, 23-3-1, 23-4-1, 23-5-1, 23-6-1, 23-7-1, 23-8-1, 23-9-1, 23-10-1, 23-11, 23-12-1, and 23-13-1.

The present disclosure provides a pharmaceutical composition for use in treating and/or preventing cancer, comprising a CBP/P300 inhibitor as an active ingredient, characterized by being administered to a subject comprising at least one selected from the group consisting of a dysfunction of an SWI/SNF complex, and lack of or attenuation of expression of an SWI/SNF complex protein.

In one embodiment, the subject comprising at least one selected from the group consisting of a dysfunction of an SWI/SNF complex, and lack of or attenuation of expression of an SWI/SNF complex protein is determined by steps comprising
(1) a step comprising at least one selected from the group consisting of a step of detecting a mutation in an SWI/SNF complex gene of a cancer cell obtained from the subject, and a step of measuring expression of an SWI/SNF complex protein, and
(2) a step of determining that the subject comprises at least one selected from the group consisting of a dysfunction of an SWI/SNF complex, and lack of or attenuation of expression of an SWI/SNF complex protein based on at least one selected from the group consisting of the presence/absence of a mutation in an SWI/SNF complex gene and a result of expression of an SWI/SNF complex protein detected in (1).

In one embodiment, the SWI/SNF complex is a BAF complex, the SWI/SNF complex gene is a BAF complex gene, and the SWI/SNF complex protein is a BAF complex protein.

In one embodiment, the BAF complex gene comprises at least one gene selected from the group consisting of an SMARC gene, an SS18-SSX fusion gene, and an ARID gene, and
the BAF complex protein comprises at least one protein selected from the group consisting of an SMARC protein, an SS18-SSX fusion protein, and an ARID protein.

In one embodiment, the BAF complex gene is an SMARC gene, and
the BAF complex protein is an SMARC protein.

In one embodiment, the SMARC gene comprises at least one gene selected from the group consisting of an SMARCB1 gene, an SMARCA2 gene, and an SMARCA4 gene, and
the SMARC protein comprises at least one protein selected from the group consisting of an SMARCB1 protein, an SMARCA2 protein, and an SMARCA4 protein.

In one embodiment, the SMARC gene is an SMARCB1 gene, and the SMARC protein is an SMARCB1 protein.

In one embodiment, the SMARC gene is an SMARCA2 gene, and the SMARC protein is an SMARCA2 protein.

In one embodiment, the SMARC gene is an SMARCA4 gene, and the SMARC protein is an SMARCA4 protein.

In one embodiment, the SMARC gene comprises an SMARCA2 gene and an SMARCA4 gene, and the SMARC protein comprises an SMARCA2 protein and an SMARCA4 protein.

In one embodiment, the cancer is SMARC deficient cancer.

In one embodiment, the SMARC deficient cancer is SMARCB1 deficient cancer.

In one embodiment, the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

In one embodiment, the SMARCB1 deficient cancer is malignant rhabdoid tumor.

In one embodiment, the SMARC deficient cancer is SMARCA2 deficient cancer.

In one embodiment, the SMARCA2 deficient cancer comprises at least one selected from the group consisting of pulmonary adenocarcinoma, large cell lung carcinoma, lung neuroendocrine tumor, esophageal cancer, gastroesophageal junction cancer, and malignant rhabdoid tumor.

In one embodiment, the SMARCA2 deficient cancer is pulmonary adenocarcinoma.

In one embodiment, the SMARC deficient cancer is SMARCA4 deficient cancer.

In one embodiment, the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

In one embodiment, the SMARCA4 deficient cancer is pulmonary adenocarcinoma.

In one embodiment, the SMARC deficient cancer is SMARCA2/A4 deficient cancer.

In one embodiment, the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

In one embodiment, the SMARCA2/A4 deficient cancer is pulmonary adenocarcinoma.

In one embodiment, the BAF complex gene is an ARID gene, and the BAF complex protein is an ARID protein.

In one embodiment, the ARID gene comprises at least one gene selected from the group consisting of an ARID1A gene and an ARID1B gene, and
the ARID protein comprises at least one protein selected from the group consisting of an ARID1A protein and an ARID1B protein.

In one embodiment, the ARID gene is an ARID1A gene, and
the ARID protein is an ARID1A protein.

In one embodiment, the ARID gene is an ARID1B gene, and
the ARID protein is an ARID1B protein.

In one embodiment, the ARID gene is an ARID1A gene and an ARID1B gene, and the ARID protein is an ARID1A protein and an ARID1B protein.

In one embodiment, the cancer is ARID deficient cancer.

In one embodiment, the ARID deficient cancer is ARID1A deficient cancer.

In one embodiment, the ARID1A deficient cancer comprises at least one selected from the group consisting of ovarian cancer, gastric cancer, bile duct cancer, pancreatic cancer, uterine cancer, neuroblastoma, colon cancer, and bladder cancer.

In one embodiment, the ARID1A deficient cancer is ovarian cancer.

In one embodiment, the ARID deficient cancer is ARID1B deficient cancer.

In one embodiment, the ARID1B deficient cancer comprises at least one selected from the group consisting of ovarian cancer, colon cancer, pancreatic cancer, liver cancer, melanoma, breast cancer, medulloblastoma, uterine cancer, bladder cancer, and gastric cancer.

In one embodiment, the ARID1B deficient cancer is ovarian cancer.

In one embodiment, the ARID deficient cancer is ARID1A/1B deficient cancer.

In one embodiment, the ARID1A/1B deficient cancer comprises at least one selected from the group consisting of ovarian cancer, colon cancer, uterine cancer, neuroblastoma, bladder cancer, and gastric cancer.

In one embodiment, the ARID1A/1B deficient cancer is ovarian cancer.

In one embodiment, the BAF complex is an SS18-SSX fusion gene, and the BAF complex protein is an SS18-SSX fusion protein.

In one embodiment, the cancer is SS18-SSX fusion cancer.

In one embodiment, the SS18-SSX fusion cancer is synovial sarcoma or Ewing's sarcoma.

In one embodiment, the SS18-SSX fusion cancer is synovial sarcoma.

In one embodiment, the CBP/P300 inhibitor reduces expression of CBP and/or P300, and/or suppresses a function of CBP and/or P300.

In one embodiment, the CBP/P300 inhibitor is a nucleic acid or a low molecular weight compound.

In one embodiment, the CBP/P300 inhibitor is a low molecular weight compound.

The present disclosure provides a pharmaceutical composition comprising a CBP/P300 inhibitor in combination with at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, other antitumor agents, and agents classified as other antitumor agents.

The present disclosure provides a pharmaceutical composition comprising a CBP/P300 inhibitor for use in treating and/or preventing cancer by concomitantly using at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, and agents classified as other antitumor agents.

The present disclosure provides a method for assisting prediction of efficacy of a CBP/P300 inhibitor on a subject, comprising at least one selected from the group consisting of detecting a dysfunction of an SWI/SNF complex in a cancer cell of the subject, and measuring expression of an SWI/SNF complex protein.

In one embodiment, the at least one selected from the group consisting of detecting a dysfunction of an SWI/SNF complex in a cancer cell, and measuring expression of an SWI/SNF complex protein is determined by steps comprising
(1) at least one selected from the group consisting of a step of detecting a mutation in an SWI/SNF complex gene of a cancer cell obtained from the subject and a step of measuring expression of an SWI/SNF complex protein, and
(2) a step of determining that the subject comprises at least one selected from the group consisting of a dysfunction of an SWI/SNF complex, and lack of or attenuation of expression of an SWI/SNF complex protein based on at least one selected from the group consisting of the presence/absence of a mutation in an SWI/SNF complex gene and a result of expression of an SWI/SNF complex protein detected in (1).

The present disclosure provides a method of using at least one selected from the group consisting of the presence/absence or level of a mutation in an SWI/SNF complex gene in a cancer cell of a subject and the presence/absence or level of expression of an SWI/SNF complex protein as an indicator for predicting efficacy of a CBP/P300 inhibitor on the subject.

In one embodiment, the SWI/SNF complex is a BAF complex, the SWI/SNF complex gene is a BAF complex gene, and the SWI/SNF complex protein is a BAF complex protein.

In one embodiment, the BAF complex gene comprises at least one gene selected from the group consisting of an SMARC gene, an SS18-SSX fusion gene, and an ARID gene, and
the BAF complex protein comprises at least one protein selected from the group consisting of an SMARC protein, an SS18-SSX fusion protein, and an ARID protein.

In one embodiment, the BAF complex gene is an SMARC gene, and
the BAF complex protein is an SMARC protein.

In one embodiment, the SMARC gene comprises at least one gene selected from the group consisting of an SMARCB1 gene, an SMARCA2 gene, and an SMARCA4 gene, and the SMARC protein comprises at least one protein selected from the group consisting of an SMARCB1 protein, an SMARCA2 protein, and an SMARCA4 protein.

In one embodiment, the SMARC gene is an SMARCB1 gene, and the SMARC protein is an SMARCB1 protein.

In one embodiment, the SMARC gene is an SMARCA2 gene, and the SMARC protein is an SMARCA2 protein.

In one embodiment, the SMARC gene is an SMARCA4 gene, and the SMARC protein is an SMARCA4 protein.

In one embodiment, the SMARC gene comprises an SMARCA2 gene and an SMARCA4 gene, and the SMARC protein comprises an SMARCA2 protein and an SMARCA4 protein.

In one embodiment, the cancer is SMARC deficient cancer.

In one embodiment, the SMARC deficient cancer is SMARCB1 deficient cancer.

In one embodiment, the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

In one embodiment, the SMARCB1 deficient cancer is malignant rhabdoid tumor.

In one embodiment, the SMARC deficient cancer is SMARCA2 deficient cancer.

In one embodiment, the SMARCA2 deficient cancer is pulmonary adenocarcinoma, large cell lung carcinoma, lung neuroendocrine tumor, esophageal cancer, gastroesophageal junction cancer, or malignant rhabdoid tumor.

In one embodiment, the SMARCA2 deficient cancer is pulmonary adenocarcinoma.

In one embodiment, the SMARC deficient cancer is SMARCA4 deficient cancer.

In one embodiment, the SMARCA4 deficient cancer comprises at least one selected from the group consisting of pulmonary adenocarcinoma, esophageal cancer, gastroesophageal junction cancer, gastric cancer, bladder cancer, squamous cell lung carcinoma, pancreatic cancer, medulloblastoma, clear cell renal cell carcinoma, liver cancer, small cell carcinoma of the ovary, mucinous ovarian tumor, endometrial cancer, uterine sarcoma, nasal and paranasal sinus cancer, rhabdoid tumor, and thoracic cavity sarcoma.

In one embodiment, the SMARCA4 deficient cancer is pulmonary adenocarcinoma.

In one embodiment, the SMARC deficient cancer is SMARCA2/A4 deficient cancer.

In one embodiment, the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

In one embodiment, the SMARCA2/A4 deficient cancer is pulmonary adenocarcinoma.

In one embodiment, the BAF complex gene is an ARID gene, and the BAF complex protein is an ARID protein.

In one embodiment, the ARID gene comprises at least one gene selected from the group consisting of an ARID1A gene and an ARID1B gene, and the ARID protein comprises at least one protein selected from the group consisting of an ARID1A protein and an ARID1B protein.

In one embodiment, the ARID gene is an ARID1A gene, and
the ARID protein is an ARID1A protein.

In one embodiment, the ARID gene is an ARID1B gene, and
the ARID protein is an ARID1B protein.

In one embodiment, the ARID gene comprises an ARID1A gene and an ARID1B gene, and the ARID protein comprises an ARID1A protein and an ARID1B protein.

In one embodiment, the cancer is ARID deficient cancer.

In one embodiment, the ARID deficient cancer is ARID1A deficient cancer.

In one embodiment, the ARID1A deficient cancer comprises at least one selected from the group consisting of ovarian cancer, gastric cancer, bile duct cancer, pancreatic cancer, uterine cancer, neuroblastoma, colon cancer, and bladder cancer.

In one embodiment, the ARID1A deficient cancer is ovarian cancer.

In one embodiment, the ARID deficient cancer is ARID1B deficient cancer.

In one embodiment, the ARID1B deficient cancer comprises at least one selected from the group consisting of ovarian cancer, colon cancer, pancreatic cancer, liver cancer, melanoma, breast cancer, medulloblastoma, uterine cancer, bladder cancer, and gastric cancer.

In one embodiment, the ARID1B deficient cancer is ovarian cancer.

In one embodiment, the ARID deficient cancer is ARID1A/1B deficient cancer.

In one embodiment, the ARID1A/1B deficient cancer comprises at least one selected from the group consisting of ovarian cancer, colon cancer, uterine cancer, neuroblastoma, bladder cancer, and gastric cancer.

In one embodiment, the ARID1A/1B deficient cancer is ovarian cancer.

In one embodiment, the BAF complex is an SS18-SSX fusion gene, and the BAF complex protein is an SS18-SSX fusion gene protein.

In one embodiment, the cancer is SS18-SSX fusion cancer.

In one embodiment, the SS18-SSX fusion cancer is synovial sarcoma or Ewing's sarcoma.

In one embodiment, the SS18-SSX fusion cancer is synovial sarcoma.

In one embodiment, the CBP/P300 inhibitor reduces expression of CBP and/or P300, and/or suppresses a function of CBP and/or P300.

In one embodiment, the CBP/P300 inhibitor is a nucleic acid or a low molecular weight compound.

In one embodiment, the CBP/P300 inhibitor is a low molecular weight compound.

The present disclosure provides a pharmaceutical composition for use in treating and/or preventing cancer, comprising an SWI/SNF complex inhibitor.

In one embodiment, the cancer is CBP/P300 deficient cancer.

In one embodiment, the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

In one embodiment, the SWI/SNF complex inhibitor is a BAF complex inhibitor.

In one embodiment, the BAF complex inhibitor is at least one inhibitor selected from the group consisting of an SMARC inhibitor and an ARID inhibitor.

In one embodiment, the BAF complex inhibitor is an SMARC inhibitor.

In one embodiment, the SMARC inhibitor comprises at least one inhibitor selected from the group consisting of an SMARCB1 inhibitor, an SMARCA2 inhibitor, an SMARCA4 inhibitor, and an SMARCA2/A4 inhibitor.

In one embodiment, the SMARC inhibitor is an SMARCB1 inhibitor.

In one embodiment, the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

In one embodiment, the SMARCB1 inhibitor is a low molecular weight compound that inhibits a function of SMARCB1.

In one embodiment, the SMARC inhibitor is an SMARCA2 inhibitor.

In one embodiment, the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

In one embodiment, the SMARCA2 inhibitor is a low molecular weight compound that inhibits a function of SMARCA2.

In one embodiment, the SMARC inhibitor is an SMARCA4 inhibitor.

In one embodiment, the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

In one embodiment, the SMARCA4 inhibitor is a low molecular weight compound that inhibits a function of SMARCA4.

In one embodiment, the SMARC inhibitor is an SMARCA2/A4 inhibitor.

In one embodiment, the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

In one embodiment, the SMARCA2/4 inhibitor is a low molecular weight compound that inhibits a function of SMARCA2 and SMARCA4.

In one embodiment, the BAF complex inhibitor is an ARID inhibitor.

In one embodiment, the ARID inhibitor comprises at least one inhibitor selected from the group consisting of an ARID1A inhibitor, an ARID1B inhibitor, and an ARID1A/1B inhibitor.

In one embodiment, the ARID inhibitor is an ARID1A inhibitor.

In one embodiment, the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

In one embodiment, the ARID1A inhibitor is a low molecular weight compound that inhibits a function of ARID1A.

In one embodiment, the ARID inhibitor is an ARID1B inhibitor.

In one embodiment, the ARID1B inhibitor is a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, and precursors thereof.

In one embodiment, the ARID1B inhibitor is a low molecular weight compound that inhibits a function of ARID1B.

In one embodiment, the ARID inhibitor is an ARID1A/1B inhibitor.

In one embodiment, the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

In one embodiment, the ARID1A/1B inhibitor is a low molecular weight compound that inhibits a function of ARID1A and ARID1B.

The present disclosure provides a pharmaceutical composition for use in treating and/or preventing cancer, comprising an SWI/SNF complex inhibitor as an active ingredient, characterized by being administered to a subject comprising at least one selected from the group consisting of a deficiency of a CBP/P300 gene, and lack of or attenuation of expression of a CBP/P300 protein.

In one embodiment, the subject comprising at least one selected from the group consisting of a deficiency of a CBP/P300 gene, and lack of or attenuation of expression of a CBP/P300 protein is determined by steps comprising
(1) at least one selected from the group consisting of a step of detecting a mutation in a CBP/P300 gene of a cancer cell obtained from the subject, and a step of measuring expression of a CBP/P300 protein, and
(2) a step of determining that the subject comprises at least one selected from the group consisting of a deficiency of a CBP/P300 gene, and lack of or attenuation of expression of a CBP/P300 gene based on at least one selected from the group consisting of the presence/absence of a mutation in a CBP/P300 gene and a result of expression of a CBP/P300 protein detected in (1).

In one embodiment, the cancer is CBP/P300 deficient cancer.

In one embodiment, the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

In one embodiment, the SWI/SNF complex inhibitor is a BAF complex inhibitor.

In one embodiment, the BAF complex inhibitor is at least one inhibitor selected from the group consisting of an SMARC inhibitor and an ARID inhibitor.

In one embodiment, the BAF complex inhibitor is an SMARC inhibitor.

In one embodiment, the SMARC inhibitor is at least one inhibitor selected from the group consisting of an SMARCB1 inhibitor, an SMARCA2 inhibitor, an SMARCA4 inhibitor, and an SMARCA2/A4 inhibitor.

In one embodiment, the SMARC inhibitor is an SMARCB1 inhibitor.

In one embodiment, the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

In one embodiment, the SMARCB1 inhibitor is a low molecular weight compound that inhibits a function of SMARCB1.

In one embodiment, the SMARC inhibitor is an SMARCA2 inhibitor.

In one embodiment, the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

In one embodiment, the SMARCA2 inhibitor is a low molecular weight compound that inhibits a function of SMARCA2.

In one embodiment, the SMARC inhibitor is an SMARCA4 inhibitor.

In one embodiment, the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

In one embodiment, the SMARCA4 inhibitor is a low molecular weight compound that inhibits a function of SMARCA4.

In one embodiment, the SMARC inhibitor is an SMARCA2/A4 inhibitor.

In one embodiment, the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

In one embodiment, the SMARCA2/4 inhibitor is a low molecular weight compound that inhibits a function of SMARCA2 and SMARCA4.

In one embodiment, the BAF complex inhibitor is an ARID inhibitor.

In one embodiment, the ARID inhibitor is at least one inhibitor selected from the group consisting of an ARID1A inhibitor, an ARID1B inhibitor, and an ARID1A/1B inhibitor.

In one embodiment, the ARID inhibitor is an ARID1A inhibitor.

In one embodiment, the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

In one embodiment, the ARID1A inhibitor is a low molecular weight compound that inhibits a function of ARID1A.

In one embodiment, the ARID inhibitor is an ARID1B inhibitor.

In one embodiment, the ARID1B inhibitor comprises at least one selected from the group consisting of a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, and precursors thereof.

In one embodiment, the ARID1B inhibitor is a low molecular weight compound that inhibits a function of ARID1B.

In one embodiment, the ARID inhibitor is an ARID1A/1B inhibitor.

In one embodiment, the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

In one embodiment, the ARID1A/1B inhibitor is a low molecular weight compound that inhibits a function of ARID1A and ARID1B.

The present disclosure provides a pharmaceutical composition comprising an SWI/SNF complex inhibitor in combination with at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, other antitumor agents, and agents classified as other antitumor agents.

The present disclosure provides a pharmaceutical composition comprising an SWI/SNF complex inhibitor for use in treating and/or preventing cancer by concomitantly using at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, and agents classified as other antitumor agents.

In one embodiment, the SWI/SNF complex inhibitor is a BAF complex inhibitor.

In one embodiment, the BAF complex inhibitor comprises at least one inhibitor selected from the group consisting of an SMARC inhibitor and an ARID inhibitor.

In one embodiment, the BAF complex inhibitor is an SMARC inhibitor.

In one embodiment, the SMARC inhibitor comprises at least one inhibitor selected from the group consisting of an SMARCB1 inhibitor, an SMARCA2 inhibitor, an SMARCA4 inhibitor, and an SMARCA2/A4 inhibitor.

In one embodiment, the SMARC inhibitor is an SMARCB1 inhibitor.

In one embodiment, the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

In one embodiment, the SMARCB1 inhibitor is a low molecular weight compound that inhibits a function of SMARCB1.

In one embodiment, the SMARC inhibitor is an SMARCA2 inhibitor.

In one embodiment, the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

In one embodiment, the SMARCA2 inhibitor is a low molecular weight compound that inhibits a function of SMARCA2.

In one embodiment, the SMARC inhibitor is an SMARCA4 inhibitor.

In one embodiment, the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

In one embodiment, the SMARCA4 inhibitor is a low molecular weight compound that inhibits a function of SMARCA4.

In one embodiment, the SMARC inhibitor is an SMARCA2/A4 inhibitor.

In one embodiment, the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

In one embodiment, the SMARCA2/4 inhibitor is a low molecular weight compound that inhibits a function of SMARCA2 and SMARCA4.

In one embodiment, the BAF complex inhibitor is an ARID inhibitor.

In one embodiment, the ARID inhibitor comprises at least one inhibitor selected from the group consisting of an ARID1A inhibitor, an ARID1B inhibitor, and an ARID1A/1B inhibitor.

In one embodiment, the ARID inhibitor is an ARID1A inhibitor.

In one embodiment, the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

In one embodiment, the ARID1A inhibitor is a low molecular weight compound that inhibits a function of ARID1A.

In one embodiment, the ARID inhibitor is an ARID1B inhibitor.

In one embodiment, the ARID1B inhibitor comprises at least one selected from the group consisting of a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, and precursors thereof.

In one embodiment, the ARID1B inhibitor is a low molecular weight compound that inhibits a function of ARID1B.

In one embodiment, the ARID inhibitor is an ARID1A/1B inhibitor.

In one embodiment, the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

In one embodiment, the ARID1A/1B inhibitor is a low molecular weight compound that inhibits a function of ARID1A and ARID1B.

The present disclosure provides a method of predicting efficacy of an SWI/SNF complex inhibitor on a subject, comprising at least one selected from the group consisting of detecting a mutation in a CBP/P300 gene of a cancer cell of the subject, and measuring expression of a CBP/P300 protein.

In one embodiment, the at least one selected from the group consisting of detecting a mutation in a CBP/P300 gene of a cancer cell, and measuring expression of a CBP/P300 protein is determined by steps comprising
(1) at least one selected from the group consisting of a step of detecting a mutation in a CBP/P300 gene of a cancer cell obtained from the subject and a step of measuring expression of a CBP/P300 gene, and
(2) a step of determining that the subject comprises at least one selected from the group consisting of a deficiency of a CBP/P300 gene, and lack of or attenuation of expression of a CBP/P300 gene based on at least one selected from the group consisting of the presence/absence of a mutation in a CBP/P300 gene and a result of expression of a CBP/P300 protein detected in (1).

In one embodiment, the cancer is CBP/P300 deficient cancer.

In one embodiment, the CBP/P300 deficient cancer comprises at least one selected from the group consisting of lung cancer, bladder cancer, lymphoma, adenoid cystic carcinoma, head and neck squamous cell carcinoma, cervical cancer, esophageal cancer, gastric cancer, melanoma, endometrial cancer, cholangiolocellular carcinoma, renal cell carcinoma, hepatocellular carcinoma, adrenal cancer, pancreatic cancer, colon cancer, prostate cancer, breast cancer, acute myeloid leukemia, ovarian cancer, oral cavity cancer, meningioma, nerve sheath tumor, and pheochromocytoma.

In one embodiment, the SWI/SNF complex inhibitor is a BAF complex inhibitor.

In one embodiment, the BAF complex inhibitor comprises at least one inhibitor selected from the group consisting of an SMARC inhibitor and an ARID inhibitor.

In one embodiment, the BAF complex inhibitor is an SMARC inhibitor.

In one embodiment, the SMARC inhibitor comprises at least one inhibitor selected from the group consisting of an SMARCB1 inhibitor, an SMARCA2 inhibitor, an SMARCA4 inhibitor, and an SMARCA2/A4 inhibitor.

In one embodiment, the SMARC inhibitor is an SMARCB1 inhibitor.

In one embodiment, the SMARCB1 inhibitor comprises at least one selected from the group consisting of a low molecular weight compound that inhibits a function of SMARCB1, an antisense nucleic acid for a transcriptional product of a gene encoding SMARCB1, a ribozyme for a transcriptional product of a gene encoding SMARCB1, a nucleic acid having RNAi activity for a transcriptional product of a gene encoding SMARCB1, and precursors thereof.

In one embodiment, the SMARCB1 inhibitor is a low molecular weight compound that inhibits a function of SMARCB1.

In one embodiment, the SMARC inhibitor is an SMARCA2 inhibitor.

In one embodiment, the SMARCA2 inhibitor comprises at least one selected from the group consisting of a low molecular weight compound that inhibits a function of SMARCA2, an antisense nucleic acid for a transcriptional product of a gene encoding SMARCA2, a ribozyme for a transcriptional product of a gene encoding SMARCA2, a nucleic acid having RNAi activity for a transcriptional product of a gene encoding SMARCA2, and precursors thereof.

In one embodiment, the SMARCA2 inhibitor is a low molecular weight compound that inhibits a function of SMARCA2.

In one embodiment, the SMARC inhibitor is an SMARCA4 inhibitor.

In one embodiment, the SMARCA4 inhibitor comprises at least one selected from the group consisting of a low molecular weight compound that inhibits a function of SMARCA4, an antisense nucleic acid for a transcriptional product of a gene encoding SMARCA4, a ribozyme for a transcriptional product of a gene encoding SMARCA4, a nucleic acid having RNAi activity for a transcriptional product of a gene encoding SMARCA4, and precursors thereof.

In one embodiment, the SMARCA4 inhibitor is a low molecular weight compound that inhibits a function of SMARCA4.

In one embodiment, the SMARC inhibitor is an SMARCA2/A4 inhibitor.

In one embodiment, the SMARCA2/4 inhibitor comprises at least one selected from the group consisting of a low molecular weight compound that inhibits a function of SMARCA2 and SMARCA4, an antisense nucleic acid for a transcriptional product of a gene encoding SMARCA2 and SMARCA4, a ribozyme for a transcriptional product of a gene encoding SMARCA2 and SMARCA4, a nucleic acid having RNAi activity for a transcriptional product of a gene encoding SMARCA2 and SMARCA4, and precursors thereof.

In one embodiment, the SMARCA2/4 inhibitor is a low molecular weight compound that inhibits a function of SMARCA2 and SMARCA4.

In one embodiment, the BAF complex inhibitor is an ARID inhibitor.

In one embodiment, the ARID inhibitor is at least one inhibitor selected from the group consisting of an ARID1A inhibitor, an ARID1B inhibitor, and an ARID1A/1B inhibitor.

In one embodiment, the ARID inhibitor is an ARID1A inhibitor.

In one embodiment, the ARID1A inhibitor comprises at least one selected from the group consisting of a low molecular weight compound that inhibits a function of ARID1A, an antisense nucleic acid for a transcriptional product of a gene encoding ARID1A, a ribozyme for a transcriptional product of a gene encoding ARID1A, a nucleic acid having RNAi activity for a transcriptional product of a gene encoding ARID1A, and precursors thereof.

In one embodiment, the ARID1A inhibitor is a low molecular weight compound that inhibits a function of ARID1A.

In one embodiment, the ARID inhibitor is an ARID1B inhibitor.

In one embodiment, the ARID1B inhibitor comprises at least one selected from the group consisting of a low molecular weight compound that inhibits a function of ARID1B, an antisense nucleic acid for a transcriptional product of a gene encoding ARID1B, a ribozyme for a transcriptional product of a gene encoding ARID1B, a nucleic acid having RNAi activity for a transcriptional product of a gene encoding ARID1B, and precursors thereof.

In one embodiment, the ARID1B inhibitor is a low molecular weight compound that inhibits a function of ARID1B.

In one embodiment, the ARID inhibitor is an ARID1A/1B inhibitor.

In one embodiment, the ARID1A/1B inhibitor comprises at least one selected from the group consisting of a low molecular weight compound that inhibits a function of ARID1A and ARID1B, an antisense nucleic acid for a transcriptional product of a gene encoding ARID1A and ARID1B, a ribozyme for a transcriptional product of a gene encoding ARID1A and ARID1B, a nucleic acid having RNAi activity for a transcriptional product of a gene encoding ARID1A and ARID1B, and precursors thereof.

In one embodiment, the ARID1A/1B inhibitor is a low molecular weight compound that inhibits a function of ARID1A and ARID1B.

As used herein, “or” is used when “at least one” of the elements listed in the sentence can be used. When explicitly described herein as “within a range of two values”, the two values themselves are included in the range.

Reference literatures such as scientific literatures, patents, and patent applications cited herein are incorporated herein by reference to the same extent that the entirety of each document is specifically described.

As described above, the present invention has been described while showing preferred embodiments to facilitate understanding. While the present invention is described hereinafter based on Examples, the above descriptions and the following Examples are not provided to limit the present invention, but for the sole purpose of exemplification. Thus, the scope of the invention is not limited to the embodiments and Examples specifically described herein and is limited only by the scope of claims.

SEQ ID NO: 1
MAENLLDGPPNPKRAKLSSPGFSANDSTDFGSLFDLENDLPDELIPNGGELGLLNSGNL
VPDAASKHKQLSELLRGGSGSSINPGIGNVSASSPVQQGLGGQAQGQPNSANMASLSAM
GKSPLSQGDSSAPSLPKQAASTSGPTPAASQALNPQAQKQVGLATSSPATSQTGPGICM
NANFNQTHPGLLNSNSGHSLINQASQGQAQVMNGSLGAAGRGRGAGMPYPTPAMQGASS
SVLAETLTQVSPQMTGHAGLNTAQAGGMAKMGITGNTSPFGQPFSQAGGQPMGATGVNP
QLASKQSMVNSLPTFPTDIKNTSVINVPNMSQMQTSVGIVPTQAIATGPTADPEKRKLI
QQQLVLLLHAHKCQRREQANGEVRACSLPHCRTMKNVLNHMTHCQAGKACQVAHCASSR
QIISHWKNCTRHDCPVCLPLKNASDKRNQQTILGSPASGIQNTIGSVGTGQQNATSLSN
PNPIDPSSMQRAYAALGLPYMNQPQTQLQPQVPGQQPAQPQTHQQMRTLNPLGNNPMNI
PAGGITTDQQPPNLISESALPTSLGATNPLMNDGSNSGNIGTLSTIPTAAPPSSTGVRK
GWHEHVTQDLRSHLVHKLVQAIFPTPDPAALKDRRMENLVAYAKKVEGDMYESANSRDE
YYHLLAEKIYKIQKELEEKRRSRLHKQGILGNQPALPAPGAQPPVIPQAQPVRPPNGPL
SLPVNRMQVSQGMNSFNPMSLGNVQLPQAPMGPRAASPMNHSVQMNSMGSVPGMAISPS
RMPQPPNMMGAHTNNMMAQAPAQSQFLPQNQFPSSSGAMSVGMGQPPAQTGVSQGQVPG
AALPNPLNMLGPQASQLPCPPVTQSPLHPTPPPASTAAGMPSLQHTTPPGMTPPQPAAP
TQPSTPVSSSGQTPTPTPGSVPSATQTQSTPTVQAAAQAQVTPQPQTPVQPPSVATPQS
SQQQPTPVHAQPPGTPLSQAAASIDNRVPTPSSVASAETNSQQPGPDVPVLEMKTETQA
EDTEPDPGESKGEPRSEMMEEDLQGASQVKEETDIAEQKSEPMEVDEKKPEVKVEVKEE
EESSSNGTASQSTSPSQPRKKIFKPEELRQALMPTLEALYRQDPESLPFRQPVDPQLLG
IPDYFDIVKNPMDLSTIKRKLDTGQYQEPWQYVDDVWLMENNAWLYNRKTSRVYKFCSK
LAEVFEQEIDPVMQSLGYCCGRKYEFSPQTLCCYGKQLCTIPRDAAYYSYQNRYHFCEK
CFTEIQGENVTLGDDPSQPQTTISKDQFEKKKNDTLDPEPFVDCKECGRKMHQICVLHY
DIIWPSGFVCDNCLKKTGRPRKENKFSAKRLQTTRLGNHLEDRVNKFLRRQNHPEAGEV
FVRVVASSDKTVEVKPGMKSRFVDSGEMSESFPYRTKALFAFEEIDGVDVCFFGMHVQE
YGSDCPPPNTRRVYISYLDSIHFFRPRCLRTAVYHEILIGYLEYVKKLGYVTGHIWACP
PSEGDDYIFHCHPPDQKIPKPKRLQEWYKKMLDKAFAERIIHDYKDIFKQATEDRLTSA
KELPYFEGDFWPNVLEESIKELEQEEEERKKEESTAASETTEGSQGDSKNAKKKNNKKT
NKNKSSISRANKKKPSMPNVSNDLSQKLYATMEKHKEVFFVIHLHAGPVINTLPPIVDP
DPLLSCDLMDGRDAFLTLARDKHWEFSSLRRSKWSTLCMLVELHTQGQDRFVYTCNECK
HHVETRWHCTVCEDYDLCINCYNTKSHAHKMVKWGLGLDDEGSSQGEPQSKSPQESRRL
SIQRCIQSLVHACQCRNANCSLPSCQKMKRVVQHTKGCKRKTNGGCPVCKQLIALCCYH
AKHCQENKCPVPFCLNIKHKLRQQQIQHRLQQAQLMRRRMATMNTRNVPQQSLPSPTSA
PPGTPTQQPSTPQTPQPPAQPQPSPVSMSPAGFPSVARTQPPTTVSTGKPTSQVPAPPP
PAQPPPAAVEAARQIEREAQQQQHLYRVNINNSMPPGRTGMGTPGSQMAPVSLNVPRPN
QVSGPVMPSMPPGQWQQAPLPQQQPMPGLPRPVISMQAQAAVAGPRMPSVQPPRSISPS
ALQDLLRTLKSPSSPQQQQQVLNILKSNPQLMAAFIKQRTAKYVANQPGMQPQPGLQSQ
PGMQPQPGMHQQPSLQNLNAMQAGVPRPGVPPQQQAMGGLNPQGQALNIMNPGHNPNMA
SMNPQYREMLRRQLLQQQQQQQQQQQQQQQQQQGSAGMAGGMAGHGQFQQPQGPGGYPP
AMQQQQRMQQHLPLQGSSMGQMAAQMGQLGQMGQPGLGADSTPNIQQALQQRILQQQQM
KQQIGSPGQPNPMSPQQHMLSGQPQASHLPGQQIATSLSNQVRSPAPVQSPRPQSQPPH
SSPSPRIQPQPSPHHVSPQTGSPHPGLAVTMASSIDQGHLGNPEQSAMLPQLNTPSRSA
LSSELSLVGDTTGDTLEKFVEGL
SEQ ID NO: 2
MAENLLDGPPNPKRAKLSSPGFSANDSTDFGSLFDLENDLPDELIPNGGELGLLNSGNL
VPDAASKHKQLSELLRGGSGSSINPGIGNVSASSPVQQGLGGQAQGQPNSANMASLSAM
GKSPLSQGDSSAPSLPKQAASTSGPTPAASQALNPQAQKQVGLATSSPATSQTGPGICM
NANFNQTHPGLLNSNSGHSLINQASQGQAQVMNGSLGAAGRGRGAGMPYPTPAMQGASS
SVLAETLTQVSPQMTGHAGLNTAQAGGMAKMGITGNTSPFGQPFSQAGGQPMGATGVNP
QLASKQSMVNSLPTFPTDIKNTSVTNVPNMSQMQTSVGIVPTQAIATGPTADPEKRKLI
QQQLVLLLHAHKCQRREQANGEVRACSLPHCRTMKNVLNHMTHCQAGKACQAILGSPAS
GIQNTIGSVGTGQQNATSLSNPNPIDPSSMQRAYAALGLPYMNQPQTQLQPQVPGQQPA
QPQTHQQMRTLNPLGNNPMNIPAGGITTDQQPPNLISESALPTSLGATNPLMNDGSNSG
NIGTLSTIPTAAPPSSTGVRKGWHEHVTQDLRSHLVHKLVQAIFPTPDPAALKDRRMEN
LVAYAKKVEGDMYESANSRDEYYHLLAEKIYKIQKELEEKRRSRLHKQGILGNQPALPA
PGAQPPVIPQAQPVRPPNGPLSLPVNRMQVSQGMNSFNPMSLGNVQLPQAPMGPRAASP
MNHSVQMNSMGSVPGMAISPSRMPQPPNMMGAHTNNMMAQAPAQSQFLPQNQFPSSSGA
MSVGMGQPPAQTGVSQGQVPGAALPNPLNMLGPQASQLPCPPVTQSPLHPTPPPASTAA
GMPSLQHTTPPGMTPPQPAAPTQPSTPVSSSGQTPTPTPGSVPSATQTQSTPTVQAAAQ
AQVTPQPQTPVQPPSVATPQSSQQQPTPVHAQPPGTPLSQAAASIDNRVPTPSSVASAE
TNSQQPGPDVPVLEMKTETQAEDTEPDPGESKGEPRSEMMEEDLQGASQVKEETDIAEQ
KSEPMEVDEKKPEVKVEVKEEEESSSNGTASQSTSPSQPRKKIFKPEELRQALMPTLEA
LYRQDPESLPFRQPVDPQLLGIPDYFDIVKNPMDLSTIKRKLDTGQYQEPWQYVDDVWL
MFNNAWLYNRKTSRVYKFCSKLAEVFEQEIDPVMQSLGYCCGRKYEFSPQTLCCYGKQL
CTIPRDAAYYSYQNRYHFCEKCFTEIQGENVTLGDDPSQPQTTISKDQFEKKKNDTLDP
EPFVDCKECGRKMHQICVLHYDIIWPSGFVCDNCLKKTGRPRKENKFSAKRLQTTRLGN
HLEDRVNKFLRRQNHPEAGEVFVRVVASSDKTVEVKPGMKSRFVDSGEMSESFPYRTKA
LFAFEEIDGVDVCFFGMHVQEYGSDCPPPNTRRVYISYLDSIHFFRPRCLRTAVYHEIL
IGYLEYVKKLGYVTGHIWACPPSEGDDYIFHCHPPDQKIPKPKRLQEWYKKMLDKAFAE
RIIHDYKDIFKQATEDRLTSAKELPYFEGDFWPNVLEESIKELEQEEEERKKEESTAAS
ETTEGSQGDSKNAKKKNNKKTNKNKSSISRANKKKPSMPNVSNDLSQKLYATMEKHKEV
FFVIHLHAGPVINTLPPIVDPDPLLSCDLMDGRDAFLTLARDKHWEFSSLRRSKWSTLC
MLVELHTQGQDRFVYTCNECKHHVETRWHCTVCEDYDLCINCYNTKSHAHKMVKWGLGL
DDEGSSQGEPQSKSPQESRRLSIQRCIQSLVHACQCRNANCSLPSCQKMKRVVQHTKGC
KRKTNGGCPVCKQLIALCCYHAKHCQENKCPVPFCLNIKHKLRQQQIQHRLQQAQLMRR
RMATMNTRNVPQQSLPSPTSAPPGTPTQQPSTPQTPQPPAQPQPSPVSMSPAGFPSVAR
TQPPTTVSTGKPTSQVPAPPPPAQPPPAAVEAARQIEREAQQQQHLYRVNINNSMPPGR
TGMGTPGSQMAPVSLNVPRPNQVSGPVMPSMPPGQWQQAPLPQQQPMPGLPRPVISMQA
QAAVAGPRMPSVQPPRSISPSALQDLLRTLKSPSSPQQQQQVLNILKSNPQLMAAFIKQ
RTAKYVANQPGMQPQPGLQSQPGMQPQPGMHQQPSLQNLNAMQAGVPRPGVPPQQQAMG
GLNPQGQALNIMNPGHNPNMASMNPQYREMLRRQLLQQQQQQQQQQQQQQQQQQGSAGM
AGGMAGHGQFQQPQGPGGYPPAMQQQQRMQQHLPLQGSSMGQMAAQMGQLGQMGQPGLG
ADSTPNIQQALQQRILQQQQMKQQIGSPGQPNPMSPQQHMLSGQPQASHLPGQQIATSL
SNQVRSPAPVQSPRPQSQPPHSSPSPRIQPQPSPHHVSPQTGSPHPGLAVTMASSIDQG
HLGNPEQSAMLPQLNTPSRSALSSELSLVGDTTGDTLEKFVEGL
SEQ ID NO: 3
MAENVVEPGPPSAKRPKLSSPALSASASDGTDFGSLFDLEHDLPDELINSTELGLTNGG
DINQLQTSLGMVQDAASKHKQLSELLRSGSSPNLNMGVGGPGQVMASQAQQSSPGLGLI
NSMVKSPMTQAGLTSPNMGMGTSGPNQGPTQSTGMMNSPVNQPAMGMNTGMNAGMNPGM
LAAGNGQGIMPNQVMNGSIGAGRGRQNMQYPNPGMGSAGNLLTEPLQQGSPQMGGQTGL
RGPQPLKMGMMNNPNPYGSPYTQNPGQQIGASGLGLQIQTKTVLSNNLSPFAMDKKAVP
GGGMPNMGQQPAPQVQQPGLVTPVAQGMGSGAHTADPEKRKLIQQQLVLLLHAHKCQRR
EQANGEVRQCNLPHCRTMKNVLNHMTHCQSGKSCQVAHCASSRQIISHWKNCTRHDCPV
CLPLKNAGDKRNQQPILTGAPVGLGNPSSLGVGQQSAPNLSTVSQIDPSSIERAYAALG
LPYQVNQMPTQPQVQAKNQQNQQPGQSPQGMRPMSNMSASPMGVNGGVGVQTPSLLSDS
MLHSAINSQNPMMSENASVPSLGPMPTAAQPSTTGIRKQWHEDITQDLRNHLVHKLVQA
IFPTPDPAALKDRRMENLVAYARKVEGDMYESANNRAEYYHLLAEKIYKIQKELEEKRR
TRLQKQNMLPNAAGMVPVSMNPGPNMGQPQPGMTSNGPLPDPSMIRGSVPNQMMPRITP
QSGLNQFGQMSMAQPPIVPRQTPPLQHHGQLAQPGALNPPMGYGPRMQQPSNQGQFLPQ
TQFPSQGMNVTNIPLAPSSGQAPVSQAQMSSSSCPVNSPIMPPGSQGSHIHCPQLPQPA
LHQNSPSPVPSRTPTPHHTPPSIGAQQPPATTIPAPVPTPPAMPPGPQSQALHPPPRQT
PTPPTTQLPQQVQPSLPAAPSADQPQQQPRSQQSTAASVPTPTAPLLPPQPATPLSQPA
VSIEGQVSNPPSTSSTEVNSQAIAEKQPSQEVKMEAKMEVDQPEPADTQPEDISESKVE
DCKMESTETEERSTELKTEIKEEEDQPSTSATQSSPAPGQSKKKIFKPEELRQALMPTL
EALYRQDPESLPFRQPVDPQLLGIPDYFDIVKSPMDLSTIKRKLDTGQYQEPWQYVDDI
WLMFNNAWLYNRKTSRVYKYCSKLSEVFEQEIDPVMQSLGYCCGRKLEFSPQTLCCYGK
QLCTIPRDATYYSYQNRYHFCEKCFNEIQGESVSLGDDPSQPQTTINKEQFSKRKNDTL
DPELFVECTECGRKMHQICVLHHEIIWPAGFVCDGCLKKSARTRKENKFSAKRLPSTRL
GTFLENRVNDFLRRQNHPESGEVTVRVVHASDKTVEVKPGMKARFVDSGEMAESFPYRT
KALFAFEEIDGVDLCFFGMHVQEYGSDCPPPNQRRVYISYLDSVHFFRPKCLRTAVYHE
ILIGYLEYVKKLGYTTGHIWACPPSEGDDYIFHCHPPDQKIPKPKRLQEWYKKMLDKAV
SERIVHDYKDIFKQATEDRLTSAKELPYFEGDFWPNVLEESIKELEQEEEERKREENTS
NESTDVTKGDSKNAKKKNNKKTSKNKSSLSRGNKKKPGMPNVSNDLSQKLYATMEKHKE
VFFVIRLIAGPAANSLPPIVDPDPLIPCDLMDGRDAFLTLARDKHLEFSSLRRAQWSTM
CMLVELHTQSQDRFVYTCNECKHHVETRWHCTVCEDYDLCITCYNTKNHDHKMEKLGLG
LDDESNNQQAAATQSPGDSRRLSIQRCIQSLVHACQCRNANCSLPSCQKMKRVVQHTKG
CKRKTNGGCPICKQLIALCCYHAKHCQENKCPVPFCLNIKQKLRQQQLQHRLQQAQMLR
RRMASMQRTGVVGQQQGLPSPTPATPTTPTGQQPTTPQTPQPTSQPQPTPPNSMPPYLP
RTQAAGPVSQGKAAGQVTPPTPPQTAQPPLPGPPPAAVEMAMQIQRAAETQRQMAHVQI
FQRPIQHQMPPMTPMAPMGMNPPPMTRGPSGHLEPGMGPTGMQQQPPWSQGGLPQPQQL
QSGMPRPAMMSVAQHGQPLNMAPQPGLGQVGISPLKPGTVSQQALQNLLRTLRSPSSPL
QQQQVLSILHANPQLLAAFIKQRAAKYANSNPQPIPGQPGMPQGQPGLQPPTMPGQQGV
HSNPAMQNMNPMQAGVQRAGLPQQQPQQQLQPPMGGMSPQAQQMNMNHNTMPSQFRDIL
RRQQMMQQQQQQGAGPGIGPGMANHNQFQQPQGVGYPPQQQQRMQHHMQQMQQGNMGQI
GQLPQALGAEAGASLQAYQQRLLQQQMGSPVQPNPMSPQQHMLPNQAQSPHLQGQQIPN
SLSNQVRSPQPVPSPRPQSQPPHSSPSPRMQPQPSPHHVSPQTSSPHPGLVAAQANPME
QGHFASPDQNSMLSQLASNPGMANLHGASATDLGLSTDNSDLNSNLSQSTLDIH
SEQ ID NO: 4
ctgcggggcgctgttgctgtggctgagatttggccgccgcctcccccacccggcctgcg
ccctccctctccctcggcgcccgcccgcccgctcgcggcccgcgctcgctcctctccct
cgcagccggcagggcccccgacccccgtccgggccctcgccggcccggccgcccgtgcc
cggggctgttttcgcgagcaggtgaaaatggctgagaacttgctggacggaccgcccaa
ccccaaaagagccaaactcagctcgcccggtttctcggcgaatgacagcacagattttg
gatcattgtttgacttggaaaatgatcttcctgatgagctgatacccaatggaggagaa
ttaggccttttaaacagtgggaaccttgttccagatgctgcttccaaacataaacaact
gtcggagcttctacgaggaggcagcggctctagtatcaacccaggaataggaaatgtga
gcgccagcagccccgtgcagcagggcctgggtggccaggctcaagggcagccgaacagt
gctaacatggccagcctcagtgccatgggcaagagccctctgagccagggagattcttc
agcccccagcctgcctaaacaggcagccagcacctctgggcccacccccgctgcctccc
aagcactgaatccgcaagcacaaaagcaagtggggctggcgactagcagccctgccacg
tcacagactggacctggtatctgcatgaatgctaactttaaccagacccacccaggcct
cctcaatagtaactctggccatagcttaattaatcaggcttcacaagggcaggcgcaag
tcatgaatggatctcttggggctgctggcagaggaaggggagctggaatgccgtaccct
actccagccatgcagggcgcctcgagcagcgtgctggctgagaccctaacgcaggtttc
cccgcaaatgactggtcacgcgggactgaacaccgcacaggcaggaggcatggccaaga
tgggaataactgggaacacaagtccatttggacagccctttagtcaagctggagggcag
ccaatgggagccactggagtgaacccccagttagccagcaaacagagcatggtcaacag
tttgcccaccttccctacagatatcaagaatacttcagtcaccaacgtgccaaatatgt
ctcagatgcaaacatcagtgggaattgtacccacacaagcaattgcaacaggccccact
gcagatcctgaaaaacgcaaactgatacagcagcagctggttctactgcttcatgctca
taagtgtcagagacgagagcaagcaaacggagaggttcgggcctgctcgctcccgcatt
gtcgaaccatgaaaaacgttttgaatcacatgacgcattgtcaggctgggaaagcctgc
caagccatcctggggtctccagctagtggaattcaaaacacaattggttctgttggcac
agggcaacagaatgccacttctttaagtaacccaaatcccatagaccccagctccatgc
agcgagcctatgctgctctcggactcccctacatgaaccagccccagacgcagctgcag
cctcaggttcctggccagcaaccagcacagcctcaaacccaccagcagatgaggactct
caaccccctgggaaataatccaatgaacattccagcaggaggaataacaacagatcagc
agcccccaaacttgatttcagaatcagctcttccgacttccctgggggccacaaaccca
ctgatgaacgatggctccaactctggtaacattggaaccctcagcactataccaacagc
agctcctccttctagcaccggtgtaaggaaaggctggcacgaacatgtcactcaggacc
tgcggagccatctagtgcataaactcgtccaagccatcttcccaacacctgatcccgca
gctctaaaggatcgccgcatggaaaacctggtagcctatgctaagaaagtggaagggga
catgtacgagtctgccaacagcagggatgaatattatcacttattagcagagaaaatct
acaagatacaaaaagaactagaagaaaaacggaggtcgcgtttacataaacaaggcatc
ttggggaaccagccagccttaccagccccgggggctcagccccctgtgattccacaggc
acaacctgtgagacctccaaatggacccctgtccctgccagtgaatcgcatgcaagttt
ctcaagggatgaattcatttaaccccatgtccttggggaacgtccagttgccacaagca
cccatgggacctcgtgcagcctccccaatgaaccactctgtccagatgaacagcatggg
ctcagtgccagggatggccatttctccttcccgaatgcctcagcctccgaacatgatgg
gtgcacacaccaacaacatgatggcccaggcgcccgctcagagccagtttctgccacag
aaccagttcccgtcatccagcggggcgatgagtgtgggcatggggcagccgccagccca
aacaggcgtgtcacagggacaggtgcctggtgctgctcttcctaaccctctcaacatgc
tggggcctcaggccagccagctaccttgccctccagtgacacagtcaccactgcaccca
acaccgcctcctgcttccacggctgctggcatgccatctctccagcacacgacaccacc
tgggatgactcctccccagccagcagctcccactcagccatcaactcctgtgtcgtctt
ccgggcagactcccaccccgactcctggctcagtgcccagtgctacccaaacccagagc
acccctacagtccaggcagcagcccaggcccaggtgaccccgcagcctcaaaccccagt
tcagcccccgtctgtggctacccctcagtcatcgcagcaacagccgacgcctgtgcacg
cccagcctcctggcacaccgctttcccaggcagcagccagcattgataacagagtccct
accccctcctcggtggccagcgcagaaaccaattcccagcagccaggacctgacgtacc
tgtgctggaaatgaagacggagacccaagcagaggacactgagcccgatcctggtgaat
ccaaaggggagcccaggtctgagatgatggaggaggatttgcaaggagcttcccaagtt
aaagaagaaacagacatagcagagcagaaatcagaaccaatggaagtggatgaaaagaa
acctgaagtgaaagtagaagttaaagaggaagaagagagtagcagtaacggcacagcct
ctcagtcaacatctccttcgcagccgcgcaaaaaaatctttaaaccagaggagttacgc
caggccctcatgccaaccctagaagcactgtatcgacaggacccagagtcattaccttt
ccggcagcctgtagatccccagctcctcggaattccagactattttgacatcgtaaaga
atcccatggacctctccaccatcaagcggaagctggacacagggcaataccaagagccc
tggcagtacgtggacgacgtctggctcatgttcaacaatgcctggctctataatcgcaa
gacatcccgagtctataagttttgcagtaagcttgcagaggtctttgagcaggaaattg
accctgtcatgcagtcccttggatattgctgtggacgcaagtatgagttttccccacag
actttgtgctgctatgggaagcagctgtgtaccattcctcgcgatgctgcctactacag
ctatcagaataggtatcatttctgtgagaagtgtttcacagagatccagggcgagaatg
tgaccctgggtgacgacccttcacagccccagacgacaatttcaaaggatcagtttgaa
aagaagaaaaatgataccttagaccccgaacctttcgttgattgcaaggagtgtggccg
gaagatgcatcagatttgcgttctgcactatgacatcatttggccttcaggttttgtgt
gcgacaactgcttgaagaaaactggcagacctcgaaaagaaaacaaattcagtgctaag
aggctgcagaccacaagactgggaaaccacttggaagaccgagtgaacaaatttttgcg
gcgccagaatcaccctgaagccggggaggtttttgtccgagtggtggccagctcagaca
agacggtggaggtcaagcccgggatgaagtcacggtttgtggattctggggaaatgtct
gaatctttcccatatcgaaccaaagctctgtttgcttttgaggaaattgacggcgtgga
tgtctgcttttttggaatgcacgtccaagaatacggctctgattgcccccctccaaaca
cgaggcgtgtgtacatttcttatctggatagtattcatttcttccggccacgttgcctc
cgcacagccgtttaccatgagatccttattggatatttagagtatgtgaagaaattagg
gtatgtgacagggcacatctgggcctgtcctccaagtgaaggagatgattacatcttcc
attgccacccacctgatcaaaaaatacccaagccaaaacgactgcaggagtggtacaaa
aagatgctggacaaggcgtttgcagagcggatcatccatgactacaaggatattttcaa
acaagcaactgaagacaggctcaccagtgccaaggaactgccctattttgaaggtgatt
tctggcccaatgtgttagaagagagcattaaggaactagaacaagaagaagaggagagg
aaaaaggaagagagcactgcagccagtgaaaccactgagggcagtcagggcgacagcaa
gaatgccaagaagaagaacaacaagaaaaccaacaagaacaaaagcagcatcagccgcg
ccaacaagaagaagcccagcatgcccaacgtgtccaatgacctgtcccagaagctgtat
gccaccatggagaagcacaaggaggtcttcttcgtgatccacctgcacgctgggcctgt
catcaacaccctgccccccatcgtcgaccccgaccccctgctcagctgtgacctcatgg
atgggcgcgacgccttcctcaccctcgccagagacaagcactgggagttctcctccttg
cgccgctccaagtggtccacgctctgcatgctggtggagctgcacacccagggccagga
ccgctttgtctacacctgcaacgagtgcaagcaccacgtggagacgcgctggcactgca
ctgtgtgcgaggactacgacctctgcatcaactgctataacacgaagagccatgcccat
aagatggtgaagtgggggctgggcctggatgacgagggcagcagccagggcgagccaca
gtcaaagagcccccaggagtcacgccggctgagcatccagcgctgcatccagtcgctgg
tgcacgcgtgccagtgccgcaacgccaactgctcgctgccatcctgccagaagatgaag
cgggtggtgcagcacaccaagggctgcaaacgcaagaccaacgggggctgcccggtgtg
caagcagctcatcgccctctgctgctaccacgccaagcactgccaagaaaacaaatgcc
ccgtgcccttctgcctcaacatcaaacacaagctccgccagcagcagatccagcaccgc
ctgcagcaggcccagctcatgcgccggcggatggccaccatgaacacccgcaacgtgcc
tcagcagagtctgccttctcctacctcagcaccgcccgggacccccacacagcagccca
gcacaccccagacgccgcagccccctgcccagccccaaccctcacccgtgagcatgtca
ccagctggcttccccagcgtggcccggactcagccccccaccacggtgtccacagggaa
gcctaccagccaggtgccggcccccccacccccggcccagccccctcctgcagcggtgg
aagcggctcggcagatcgagcgtgaggcccagcagcagcagcacctgtaccgggtgaac
atcaacaacagcatgcccccaggacgcacgggcatggggaccccggggagccagatggc
ccccgtgagcctgaatgtgccccgacccaaccaggtgagcgggcccgtcatgcccagca
tgcctcccgggcagtggcagcaggcgccccttccccagcagcagcccatgccaggcttg
cccaggcctgtgatatccatgcaggcccaggcggccgtggctgggccccggatgcccag
cgtgcagccacccaggagcatctcacccagcgctctgcaagacctgctgcggaccctga
agtcgcccagctcccctcagcagcaacagcaggtgctgaacattctcaaatcaaacccg
cagctaatggcagctttcatcaaacagcgcacagccaagtacgtggccaatcagcccgg
catgcagccccagcctggcctccagtcccagcccggcatgcaaccccagcctggcatgc
accagcagcccagcctgcagaacctgaatgccatgcaggctggcgtgccgcggcccggt
gtgcctccacagcagcaggcgatgggaggcctgaacccccagggccaggccttgaacat
catgaacccaggacacaaccccaacatggcgagtatgaatccacagtaccgagaaatgt
tacggaggcagctgctgcagcagcagcagcaacagcagcagcaacaacagcagcaacag
cagcagcagcaagggagtgccggcatggctgggggcatggcggggcacggccagttcca
gcagcctcaaggacccggaggctacccaccggccatgcagcagcagcagcgcatgcagc
agcatctccccctccagggcagctccatgggccagatggcggctcagatgggacagctt
ggccagatggggcagccggggctgggggcagacagcacccccaacatccagcaagccct
gcagcagcggattctgcagcaacagcagatgaagcagcagattgggtccccaggccagc
cgaaccccatgagcccccagcaacacatgctctcaggacagccacaggcctcgcatctc
cctggccagcagatcgccacgtcccttagtaaccaggtgcggtctccagcccctgtcca
gtctccacggccccagtcccagcctccacattccagcccgtcaccacggatacagcccc
agccttcgccacaccacgtctcaccccagactggttccccccaccccggactcgcagtc
accatggccagctccatagatcagggacacttggggaaccccgaacagagtgcaatgct
cccccagctgaacacccccagcaggagtgcgctgtccagcgaactgtccctggtcgggg
acaccacgggggacacgctagagaagtttgtggagggcttgtagcattgtgagagcatc
accttttccctttcatgttcttggaccttttgtactgaaaatccaggcatctaggttct
ttttattcctagatggaactgcgacttccgagccatggaagggtggattgatgtttaaa
gaaacaatacaaagaatatatttttttgttaaaaaccagttgatttaaatatctggtct
ctctctttggtttttttttggcgggggggtggggggggttcttttttttccgttttgtt
tttgtttggggggaggggggttttgtttggattctttttgtcgtcattgctggtgactc
atgcctttttttaacgggaaaaacaagttcattatattcatattttttatttgtatttt
caagactttaaacatttatgtttaaaagtaagaagaaaaataatattcagaactgattc
ctgaaataatgcaagcttataatgtatcccgataactttgtgatgtttcgggaagattt
ttttctatagtgaactctgtgggcgtctcccagtattaccctggatgataggaattgac
tccggcgtgcacacacgtacacacccacacacatctatctatacataatggctgaagcc
aaacttgtcttgcagatgtagaaattgttgctttgtttctctgataaaactggttttag
acaaaaaatagggatgatcactcttagaccatgctaatgttactagagaagaagccttc
ttttctttcttctatgtgaaacttgaaatgaggaaaagcaattctagtgtaaatcatgc
aagcgctctaattcctataaatacgaaactcgagaagattcaatcactgtatagaatgg
taaaataccaactcatttcttatatcatattgttaaataaactgtgtgcaacagacaaa
aagggtggtccttcttgaattcatgtacatggtattaacacttagtgttcggggttttt
tgttatgaaaatgctgttttcaacattgtatttggactatgcatgtgttttttccccat
tgtatataaagtaccgcttaaaattgatataaattactgaggtttttaacatgtattct
gttctttaagatccctgtaagaatgtttaaggtttttatttatttatatatattttttg
agtctgttctttgtaagacatggttctggttgttcgctcatagcggagaggctggggct
gcggttgtggttgtggcggcgtgggtggtggctgggaactgtggcccaggcttagcggc
cgcccggaggcttttcttcccggagactgaggtgggcgactgaggtgggcggctcagcg
ttggccccacacattcgaggctcacaggtgattgtcgctcacacagttagggtcgtcag
ttggtctgaaactgcatttggcccactcctccatcctccctgtccgtcgtagctgccac
ccccagaggcggcgcttcttcccgtgttcaggcggctccccccccccgtacacgactcc
cagaatctgaggcagagagtgctccaggctcgcgaggtgctttctgacttccccccaaa
tcctgccgctgccgcgcagcatgtcccgtgtggcgtttgaggaaatgctgagggacaga
caccttggagcaccagctccggtccctgttacagtgagaaaggtcccccacttcggggg
atacttgcacttagccacatggtcctgcctcccttggagtccagttccaggctccctta
ctgagtgggtgagacaagttcacaaaaaccgtaaaactgagaggaggaccatgggcagg
ggagctgaagttcatcccctaagtctaccacccccagcacccagagaacccactttatc
cctagtcccccaacaaaggctggtctaggtgggggtgatggtaattttagaaatcacgc
cccaaatagcttccgtttgggcccttacattcacagataggttttaaatagctgaatac
ttggtttgggaatctgaattcgaggaacctttctaagaagttggaaaggtccgatctag
ttttagcacagagctttgaaccttgagttataaaatgcagaataattcaagtaaaaata
agaccaccatctggcacccctgaccagcccccattcaccccatcccaggaggggaagca
caggccgggcctccggtggagattgctgccactgctcggcctgctgggttcttaacctc
cagtgtcctcttcatcttttccacccgtagggaaaccttgagccatgtgttcaaacaag
aagtggggctagagcccgagagcagcagctctaagcccacactcagaaagtggcgccct
cctggttgtgcagccttttaatgtgggcagtggaggggcctctgtttcaggttatcctg
gaattcaaaacgttatgtaccaacctcatcctctttggagtctgcatcctgtgcaaccg
tcttgggcaatccagatgtcgaaggatgtgaccgagagcatggtctgtggatgctaacc
ctaagtttgtcgtaaggaaatttctgtaagaaacctggaaagccccaacgctgtgtctc
atgctgtatacttaagaggagaagaaaaagtcctatatttgtgatcaaaaagaggaaac
ttgaaatgtgatggtgtttataataaaagatggtaaaactacttggattcaaa
SEQ ID NO: 5
aattgaggaatcaacagccgccatcttgtcgcggacccgaccggggcttcgagcgcgat
ctactcggccccgccggtcccgggccccacaaccgcccgcgcaccccgctccgcccggc
cggcccgctccgcccggccctcggcgcccgccccggcggccccgctcgcctctcggctc
ggcctcccggagcccggcggcggcggcggcggcagcggcggcggcggcggcggaacggg
gggtgggggggccgcggcggcggcggcgaccccgctcggcgcattgtttttcctcacgg
cggcggcggcggcgggccgcgggccgggagcggagcccggagccccctcgtcgtcgggc
cgcgagcgaattcattaagtggggcgcggggggggagcgaggcggcggcggcggcggca
ccatgttctcggggactgcctgagccgcccggccgggcgccgtcgctgccagccgggcc
cgggggggcggccgggccgccggggcgcccccaccgcggagtgtcgcgctcgggaggcg
ggcaggggatgagggggccgcggccggcggcggcggcggcggccgggggcgggcggtga
gcgctgcggggcgctgttgctgtggctgagatttggccgccgcctcccccacccggcct
gcgccctccctctccctcggcgcccgcccgcccgctcgcggcccgcgctcgctcctctc
cctcgcagccggcagggcccccgacccccgtccgggccctcgccggcccggccgcccgt
gcccggggctgttttcgcgagcaggtgaaaatggctgagaacttgctggacggaccgcc
caaccccaaaagagccaaactcagctcgcccggtttctcggcgaatgacagcacagatt
ttggatcattgtttgacttggaaaatgatcttcctgatgagctgatacccaatggagga
gaattaggccttttaaacagtgggaaccttgttccagatgctgcttccaaacataaaca
actgtcggagcttctacgaggaggcagcggctctagtatcaacccaggaataggaaatg
tgagcgccagcagccccgtgcagcagggcctgggtggccaggctcaagggcagccgaac
agtgctaacatggccagcctcagtgccatgggcaagagccctctgagccagggagattc
ttcagcccccagcctgcctaaacaggcagccagcacctctgggcccacccccgctgcct
cccaagcactgaatccgcaagcacaaaagcaagtggggctggcgactagcagccctgcc
acgtcacagactggacctggtatctgcatgaatgctaactttaaccagacccacccagg
cctcctcaatagtaactctggccatagcttaattaatcaggcttcacaagggcaggcgc
aagtcatgaatggatctcttggggctgctggcagaggaaggggagctggaatgccgtac
cctactccagccatgcagggcgcctcgagcagcgtgctggctgagaccctaacgcaggt
ttccccgcaaatgactggtcacgcgggactgaacaccgcacaggcaggaggcatggcca
agatgggaataactgggaacacaagtccatttggacagccctttagtcaagctggaggg
cagccaatgggagccactggagtgaacccccagttagccagcaaacagagcatggtcaa
cagtttgcccaccttccctacagatatcaagaatacttcagtcaccaacgtgccaaata
tgtctcagatgcaaacatcagtgggaattgtacccacacaagcaattgcaacaggcccc
actgcagatcctgaaaaacgcaaactgatacagcagcagctggttctactgcttcatgc
tcataagtgtcagagacgagagcaagcaaacggagaggttcgggcctgctcgctcccgc
attgtcgaaccatgaaaaacgttttgaatcacatgacgcattgtcaggctgggaaagcc
tgccaagttgcccattgtgcatcttcacgacaaatcatctctcattggaagaactgcac
acgacatgactgtcctgtttgcctccctttgaaaaatgccagtgacaagcgaaaccaac
aaaccatcctggggtctccagctagtggaattcaaaacacaattggttctgttggcaca
gggcaacagaatgccacttctttaagtaacccaaatcccatagaccccagctccatgca
gcgagcctatgctgctctcggactcccctacatgaaccagccccagacgcagctgcagc
ctcaggttcctggccagcaaccagcacagcctcaaacccaccagcagatgaggactctc
aaccccctgggaaataatccaatgaacattccagcaggaggaataacaacagatcagca
gcccccaaacttgatttcagaatcagctcttccgacttccctgggggccacaaacccac
tgatgaacgatggctccaactctggtaacattggaaccctcagcactataccaacagca
gctcctccttctagcaccggtgtaaggaaaggctggcacgaacatgtcactcaggacct
gcggagccatctagtgcataaactcgtccaagccatcttcccaacacctgatcccgcag
ctctaaaggatcgccgcatggaaaacctggtagcctatgctaagaaagtggaaggggac
atgtacgagtctgccaacagcagggatgaatattatcacttattagcagagaaaatcta
caagatacaaaaagaactagaagaaaaacggaggtcgcgtttacataaacaaggcatct
tggggaaccagccagccttaccagccccgggggctcagccccctgtgattccacaggca
caacctgtgagacctccaaatggacccctgtccctgccagtgaatcgcatgcaagtttc
tcaagggatgaattcatttaaccccatgtccttggggaacgtccagttgccacaagcac
ccatgggacctcgtgcagcctccccaatgaaccactctgtccagatgaacagcatgggc
tcagtgccagggatggccatttctccttcccgaatgcctcagcctccgaacatgatggg
tgcacacaccaacaacatgatggcccaggcgcccgctcagagccagtttctgccacaga
accagttcccgtcatccagcggggcgatgagtgtgggcatggggcagccgccagcccaa
acaggcgtgtcacagggacaggtgcctggtgctgctcttcctaaccctctcaacatgct
ggggcctcaggccagccagctaccttgccctccagtgacacagtcaccactgcacccaa
caccgcctcctgcttccacggctgctggcatgccatctctccagcacacgacaccacct
gggatgactcctccccagccagcagctcccactcagccatcaactcctgtgtcgtcttc
cgggcagactcccaccccgactcctggctcagtgcccagtgctacccaaacccagagca
cccctacagtccaggcagcagcccaggcccaggtgaccccgcagcctcaaaccccagtt
cagcccccgtctgtggctacccctcagtcatcgcagcaacagccgacgcctgtgcacgc
ccagcctcctggcacaccgctttcccaggcagcagccagcattgataacagagtcccta
ccccctcctcggtggccagcgcagaaaccaattcccagcagccaggacctgacgtacct
gtgctggaaatgaagacggagacccaagcagaggacactgagcccgatcctggtgaatc
caaaggggagcccaggtctgagatgatggaggaggatttgcaaggagcttcccaagtta
aagaagaaacagacatagcagagcagaaatcagaaccaatggaagtggatgaaaagaaa
cctgaagtgaaagtagaagttaaagaggaagaagagagtagcagtaacggcacagcctc
tcagtcaacatctccttcgcagccgcgcaaaaaaatctttaaaccagaggagttacgcc
aggccctcatgccaaccctagaagcactgtatcgacaggacccagagtcattacctttc
cggcagcctgtagatccccagctcctcggaattccagactattttgacatcgtaaagaa
tcccatggacctctccaccatcaagcggaagctggacacagggcaataccaagagccct
ggcagtacgtggacgacgtctggctcatgttcaacaatgcctggctctataatcgcaag
acatcccgagtctataagttttgcagtaagcttgcagaggtctttgagcaggaaattga
ccctgtcatgcagtcccttggatattgctgtggacgcaagtatgagttttccccacaga
ctttgtgctgctatgggaagcagctgtgtaccattcctcgcgatgctgcctactacagc
tatcagaataggtatcatttctgtgagaagtgtttcacagagatccagggcgagaatgt
gaccctgggtgacgacccttcacagccccagacgacaatttcaaaggatcagtttgaaa
agaagaaaaatgataccttagaccccgaacctttcgttgattgcaaggagtgtggccgg
aagatgcatcagatttgcgttctgcactatgacatcatttggccttcaggttttgtgtg
cgacaactgcttgaagaaaactggcagacctcgaaaagaaaacaaattcagtgctaaga
ggctgcagaccacaagactgggaaaccacttggaagaccgagtgaacaaatttttgcgg
cgccagaatcaccctgaagccggggaggtttttgtccgagtggtggccagctcagacaa
gacggtggaggtcaagcccgggatgaagtcacggtttgtggattctggggaaatgtctg
aatctttcccatatcgaaccaaagctctgtttgcttttgaggaaattgacggcgtggat
gtctgcttttttggaatgcacgtccaagaatacggctctgattgcccccctccaaacac
gaggcgtgtgtacatttcttatctggatagtattcatttcttccggccacgttgcctcc
gcacagccgtttaccatgagatccttattggatatttagagtatgtgaagaaattaggg
tatgtgacagggcacatctgggcctgtcctccaagtgaaggagatgattacatcttcca
ttgccacccacctgatcaaaaaatacccaagccaaaacgactgcaggagtggtacaaaa
agatgctggacaaggcgtttgcagagcggatcatccatgactacaaggatattttcaaa
caagcaactgaagacaggctcaccagtgccaaggaactgccctattttgaaggtgattt
ctggcccaatgtgttagaagagagcattaaggaactagaacaagaagaagaggagagga
aaaaggaagagagcactgcagccagtgaaaccactgagggcagtcagggcgacagcaag
aatgccaagaagaagaacaacaagaaaaccaacaagaacaaaagcagcatcagccgcgc
caacaagaagaagcccagcatgcccaacgtgtccaatgacctgtcccagaagctgtatg
ccaccatggagaagcacaaggaggtcttcttcgtgatccacctgcacgctgggcctgtc
atcaacaccctgccccccatcgtcgaccccgaccccctgctcagctgtgacctcatgga
tgggcgcgacgccttcctcaccctcgccagagacaagcactgggagttctcctccttgc
gccgctccaagtggtccacgctctgcatgctggtggagctgcacacccagggccaggac
cgctttgtctacacctgcaacgagtgcaagcaccacgtggagacgcgctggcactgcac
tgtgtgcgaggactacgacctctgcatcaactgctataacacgaagagccatgcccata
agatggtgaagtgggggctgggcctggatgacgagggcagcagccagggcgagccacag
tcaaagagcccccaggagtcacgccggctgagcatccagcgctgcatccagtcgctggt
gcacgcgtgccagtgccgcaacgccaactgctcgctgccatcctgccagaagatgaagc
gggtggtgcagcacaccaagggctgcaaacgcaagaccaacgggggctgcccggtgtgc
aagcagctcatcgccctctgctgctaccacgccaagcactgccaagaaaacaaatgccc
cgtgcccttctgcctcaacatcaaacacaagctccgccagcagcagatccagcaccgcc
tgcagcaggcccagctcatgcgccggcggatggccaccatgaacacccgcaacgtgcct
cagcagagtctgccttctcctacctcagcaccgcccgggacccccacacagcagcccag
cacaccccagacgccgcagccccctgcccagccccaaccctcacccgtgagcatgtcac
cagctggcttccccagcgtggcccggactcagccccccaccacggtgtccacagggaag
cctaccagccaggtgccggcccccccacccccggcccagccccctcctgcagcggtgga
agcggctcggcagatcgagcgtgaggcccagcagcagcagcacctgtaccgggtgaaca
tcaacaacagcatgcccccaggacgcacgggcatggggaccccggggagccagatggcc
cccgtgagcctgaatgtgccccgacccaaccaggtgagcgggcccgtcatgcccagcat
gcctcccgggcagtggcagcaggcgccccttccccagcagcagcccatgccaggcttgc
ccaggcctgtgatatccatgcaggcccaggcggccgtggctgggccccggatgcccagc
gtgcagccacccaggagcatctcacccagcgctctgcaagacctgctgcggaccctgaa
gtcgcccagctcccctcagcagcaacagcaggtgctgaacattctcaaatcaaacccgc
agctaatggcagctttcatcaaacagcgcacagccaagtacgtggccaatcagcccggc
atgcagccccagcctggcctccagtcccagcccggcatgcaaccccagcctggcatgca
ccagcagcccagcctgcagaacctgaatgccatgcaggctggcgtgccgcggcccggtg
tgcctccacagcagcaggcgatgggaggcctgaacccccagggccaggccttgaacatc
atgaacccaggacacaaccccaacatggcgagtatgaatccacagtaccgagaaatgtt
acggaggcagctgctgcagcagcagcagcaacagcagcagcaacaacagcagcaacagc
agcagcagcaagggagtgccggcatggctgggggcatggcggggcacggccagttccag
cagcctcaaggacccggaggctacccaccggccatgcagcagcagcagcgcatgcagca
gcatctccccctccagggcagctccatgggccagatggcggctcagatgggacagcttg
gccagatggggcagccggggctgggggcagacagcacccccaacatccagcaagccctg
cagcagcggattctgcagcaacagcagatgaagcagcagattgggtccccaggccagcc
gaaccccatgagcccccagcaacacatgctctcaggacagccacaggcctcgcatctcc
ctggccagcagatcgccacgtcccttagtaaccaggtgcggtctccagcccctgtccag
tctccacggccccagtcccagcctccacattccagcccgtcaccacggatacagcccca
gccttcgccacaccacgtctcaccccagactggttccccccaccccggactcgcagtca
ccatggccagctccatagatcagggacacttggggaaccccgaacagagtgcaatgctc
ccccagctgaacacccccagcaggagtgcgctgtccagcgaactgtccctggtcgggga
caccacgggggacacgctagagaagtttgtggagggcttgtagcattgtgagagcatca
ccttttccctttcatgttcttggaccttttgtactgaaaatccaggcatctaggttctt
tttattcctagatggaactgcgacttccgagccatggaagggtggattgatgtttaaag
aaacaatacaaagaatatatttttttgttaaaaaccagttgatttaaatatctggtctc
tctctttggtttttttttggcgggggggtggggggggttcttttttttccgttttgttt
ttgtttggggggaggggggttttgtttggattctttttgtcgtcattgctggtgactca
tgcctttttttaacgggaaaaacaagttcattatattcatattttttatttgtattttc
aagactttaaacatttatgtttaaaagtaagaagaaaaataatattcagaactgattcc
tgaaataatgcaagcttataatgtatcccgataactttgtgatgtttcgggaagatttt
tttctatagtgaactctgtgggcgtctcccagtattaccctggatgataggaattgact
ccggcgtgcacacacgtacacacccacacacatctatctatacataatggctgaagcca
aacttgtcttgcagatgtagaaattgttgctttgtttctctgataaaactggttttaga
caaaaaatagggatgatcactcttagaccatgctaatgttactagagaagaagccttct
tttctttcttctatgtgaaacttgaaatgaggaaaagcaattctagtgtaaatcatgca
agcgctctaattcctataaatacgaaactcgagaagattcaatcactgtatagaatggt
aaaataccaactcatttcttatatcatattgttaaataaactgtgtgcaacagacaaaa
agggtggtccttcttgaattcatgtacatggtattaacacttagtgttcggggtttttt
gttatgaaaatgctgttttcaacattgtatttggactatgcatgtgttttttccccatt
gtatataaagtaccgcttaaaattgatataaattactgaggtttttaacatgtattctg
ttctttaagatccctgtaagaatgtttaaggtttttatttatttatatatattttttga
gtctgttctttgtaagacatggttctggttgttcgctcatagcggagaggctggggctg
cggttgtggttgtggcggcgtgggtggtggctgggaactgtggcccaggcttagcggcc
gcccggaggcttttcttcccggagactgaggtgggcgactgaggtgggcggctcagcgt
tggccccacacattcgaggctcacaggtgattgtcgctcacacagttagggtcgtcagt
tggtctgaaactgcatttggcccactcctccatcctccctgtccgtcgtagctgccacc
cccagaggcggcgcttcttcccgtgttcaggcggctccccccccccgtacacgactccc
agaatctgaggcagagagtgctccaggctcgcgaggtgctttctgacttccccccaaat
cctgccgctgccgcgcagcatgtcccgtgtggcgtttgaggaaatgctgagggacagac
accttggagcaccagctccggtccctgttacagtgagaaaggtcccccacttcggggga
tacttgcacttagccacatggtcctgcctcccttggagtccagttccaggctcccttac
tgagtgggtgagacaagttcacaaaaaccgtaaaactgagaggaggaccatgggcaggg
gagctgaagttcatcccctaagtctaccacccccagcacccagagaacccactttatcc
ctagtcccccaacaaaggctggtctaggtgggggtgatggtaattttagaaatcacgcc
ccaaatagcttccgtttgggcccttacattcacagataggttttaaatagctgaatact
tggtttgggaatctgaattcgaggaacctttctaagaagttggaaaggtccgatctagt
tttagcacagagctttgaaccttgagttataaaatgcagaataattcaagtaaaaataa
gaccaccatctggcacccctgaccagcccccatt
caccccatcccaggaggggaagcacaggccgggcctccggtggagattgctgccactgc
tcggcctgctgggttcttaacctccagtgtcctcttcatcttttccacccgtagggaaa
ccttgagccatgtgttcaaacaagaagtggggctagagcccgagagcagcagctctaag
cccacactcagaaagtggcgccctcctggttgtgcagccttttaatgtgggcagtggag
gggcctctgtttcaggttatcctggaattcaaaacgttatgtaccaacctcatcctctt
tggagtctgcatcctgtgcaaccgtcttgggcaatccagatgtcgaaggatgtgaccga
gagcatggtctgtggatgctaaccctaagtttgtcgtaaggaaatttctgtaagaaacc
tggaaagccccaacgctgtgtctcatgctgtatacttaagaggagaagaaaaagtccta
tatttgtgatcaaaaagaggaaacttgaaatgtgatggtgtttataataaaagatggta
aaactacttggattcaaa
SEQ ID NO: 6
gagaaggaggaggacagcgccgaggaggaagaggttgatggcggcggcggagctccgag
agacctcggctgggcaggggccggccgtggcgggccggggactgcgcctctagagccgc
gagttctcgggaattcgccgcagcggacgcgctcggcgaatttgtgctcttgtgccctc
ctccgggcttgggcccaggcccggcccctcgcacttgcccttaccttttctatcgagtc
cgcatccctctccagccactgcgacccggcgaagagaaaaaggaacttcccccaccccc
tcgggtgccgtcggagccccccagcccacccctgggtgcggcgcggggaccccgggccg
aagaagagatttcctgaggattctggttttcctcgcttgtatctccgaaagaattaaaa
atggccgagaatgtggtggaaccggggccgccttcagccaagcggcctaaactctcatc
tccggccctctcggcgtccgccagcgatggcacagattttggctctctatttgacttgg
agcacgacttaccagatgaattaatcaactctacagaattgggactaaccaatggtggt
gatattaatcagcttcagacaagtcttggcatggtacaagatgcagcttctaaacataa
acagctgtcagaattgctgcgatctggtagttcccctaacctcaatatgggagttggtg
gcccaggtcaagtcatggccagccaggcccaacagagcagtcctggattaggtttgata
aatagcatggtcaaaagcccaatgacacaggcaggcttgacttctcccaacatggggat
gggcactagtggaccaaatcagggtcctacgcagtcaacaggtatgatgaacagtccag
taaatcagcctgccatgggaatgaacacagggatgaatgcgggcatgaatcctggaatg
ttggctgcaggcaatggacaagggataatgcctaatcaagtcatgaacggttcaattgg
agcaggccgagggcgacagaatatgcagtacccaaacccaggcatgggaagtgctggca
acttactgactgagcctcttcagcagggctctccccagatgggaggacaaacaggattg
agaggcccccagcctcttaagatgggaatgatgaacaaccccaatccttatggttcacc
atatactcagaatcctggacagcagattggagccagtggccttggtctccagattcaga
caaaaactgtactatcaaataacttatctccatttgctatggacaaaaaggcagttcct
ggtggaggaatgcccaacatgggtcaacagccagccccgcaggtccagcagccaggcct
ggtgactccagttgcccaagggatgggttctggagcacatacagctgatccagagaagc
gcaagctcatccagcagcagcttgttctccttttgcatgctcacaagtgccagcgccgg
gaacaggccaatggggaagtgaggcagtgcaaccttccccactgtcgcacaatgaagaa
tgtcctaaaccacatgacacactgccagtcaggcaagtcttgccaagtggcacactgtg
catcttctcgacaaatcatttcacactggaagaattgtacaagacatgattgtcctgtg
tgtctccccctcaaaaatgctggtgataagagaaatcaacagccaattttgactggagc
acccgttggacttggaaatcctagctctctaggggtgggtcaacagtctgcccccaacc
taagcactgttagtcagattgatcccagctccatagaaagagcctatgcagctcttgga
ctaccctatcaagtaaatcagatgccgacacaaccccaggtgcaagcaaagaaccagca
gaatcagcagcctgggcagtctccccaaggcatgcggcccatgagcaacatgagtgcta
gtcctatgggagtaaatggaggtgtaggagttcaaacgccgagtcttctttctgactca
atgttgcattcagccataaattctcaaaacccaatgatgagtgaaaatgccagtgtgcc
ctccctgggtcctatgccaacagcagctcaaccatccactactggaattcggaaacagt
ggcacgaagatattactcaggatcttcgaaatcatcttgttcacaaactcgtccaagcc
atatttcctacgccggatcctgctgctttaaaagacagacggatggaaaacctagttgc
atatgctcggaaagttgaaggggacatgtatgaatctgcaaacaatcgagcggaatact
accaccttctagctgagaaaatctataagatccagaaagaactagaagaaaaacgaagg
accagactacagaagcagaacatgctaccaaatgctgcaggcatggttccagtttccat
gaatccagggcctaacatgggacagccgcaaccaggaatgacttctagtttgaatcaat
ttggccagatgagcatggcccagccccctattgtaccccggcaaacccctcctcttcag
caccatggacagttggctcaacctggagctctcaacccgcctatgggctatgggcctcg
tatgcaacagccttccaaccagggccagttccttcctcagactcagttcccatcacagg
gaatgaatgtaacaaatatccctttggctccgtccagcggtcaagctccagtgtctcaa
gcacaaatgtctagttcttcctgcccggtgaactctcctataatgcctccagggtctca
ggggagccacattcactgtccccagcttcctcaaccagctcttcatcagaattcaccct
cgcctgtacctagtcgtacccccacccctcaccatactcccccaagcataggggctcag
cagccaccagcaacaacaattccagcccctgttcctacacctcctgccatgccacctgg
gccacagtcccaggctctacatccccctccaaggcagacacctacaccaccaacaacac
aacttccccaacaagtgcagccttcacttcctgctgcaccttctgctgaccagccccag
cagcagcctcgctcacagcagagcacagcagcgtctgttcctaccccaacagcaccgct
gcttcctccgcagcctgcaactccactttcccagccagctgtaagcattgaaggacagg
tatcaaatcctccatctactagtagcacagaagtgaattctcaggccattgctgagaag
cagccttcccaggaagtgaagatggaggccaaaatggaagtggatcaaccagaaccagc
agatactcagccggaggatatttcagagtctaaagtggaagactgtaaaatggaatcta
ccgaaacagaagagagaagcactgagttaaaaactgaaataaaagaggaggaagaccag
ccaagtacttcagctacccagtcatctccggctccaggacagtcaaagaaaaagatttt
caaaccagaagaactacgacaggcactgatgccaactttggaggcactttaccgtcagg
atccagaatcccttccctttcgtcaacctgtggaccctcagcttttaggaatccctgat
tactttgatattgtgaagagccccatggatctttctaccattaagaggaagttagacac
tggacagtatcaggagccctggcagtatgtcgatgatatttggcttatgttcaataatg
cctggttatataaccggaaaacatcacgggtatacaaatactgctccaagctctctgag
gtctttgaacaagaaattgacccagtgatgcaaagccttggatactgttgtggcagaaa
gttggagttctctccacagacactgtgttgctacggcaaacagttgtgcacaatacctc
gtgatgccacttattacagttaccagaacaggtatcatttctgtgagaagtgtttcaat
gagatccaaggggagagcgtttctttgggggatgacccttcccagcctcaaactacaat
aaataaagaacaattttccaagagaaaaaatgacacactggatcctgaactgtttgttg
aatgtacagagtgcggaagaaagatgcatcagatctgtgtccttcaccatgagatcatc
tggcctgctggattcgtctgtgatggctgtttaaagaaaagtgcacgaactaggaaaga
aaataagttttctgctaaaaggttgccatctaccagacttggcacctttctagagaatc
gtgtgaatgactttctgaggcgacagaatcaccctgagtcaggagaggtcactgttaga
gtagttcatgcttctgacaaaaccgtggaagtaaaaccaggcatgaaagcaaggtttgt
ggacagtggagagatggcagaatcctttccataccgaaccaaagccctctttgcctttg
aagaaattgatggtgttgacctgtgcttctttggcatgcatgttcaagagtatggctct
gactgccctccacccaaccagaggagagtatacatatcttacctcgatagtgttcattt
cttccgtcctaaatgcttgaggactgcagtctatcatgaaatcctaattggatatttag
aatatgtcaagaaattaggttacacaacagggcatatttgggcatgtccaccaagtgag
ggagatgattatatcttccattgccatcctcctgaccagaagatacccaagcccaagcg
actgcaggaatggtacaaaaaaatgcttgacaaggctgtatcagagcgtattgtccatg
actacaaggatatttttaaacaagctactgaagatagattaacaagtgcaaaggaattg
ccttatttcgagggtgatttctggcccaatgttctggaagaaagcattaaggaactgga
acaggaggaagaagagagaaaacgagaggaaaacaccagcaatgaaagcacagatgtga
ccaagggagacagcaaaaatgctaaaaagaagaataataagaaaaccagcaaaaataag
agcagcctgagtaggggcaacaagaagaaacccgggatgcccaatgtatctaacgacct
ctcacagaaactatatgccaccatggagaagcataaagaggtcttctttgtgatccgcc
tcattgctggccctgctgccaactccctgcctcccattgttgatcctgatcctctcatc
ccctgcgatctgatggatggtcgggatgcgtttctcacgctggcaagggacaagcacct
ggagttctcttcactccgaagagcccagtggtccaccatgtgcatgctggtggagctgc
acacgcagagccaggaccgctttgtctacacctgcaatgaatgcaagcaccatgtggag
acacgctggcactgtactgtctgtgaggattatgacttgtgtatcacctgctataacac
taaaaaccatgaccacaaaatggagaaactaggccttggcttagatgatgagagcaaca
accagcaggctgcagccacccagagcccaggcgattctcgccgcctgagtatccagcgc
tgcatccagtctctggtccatgcttgccagtgtcggaatgccaattgctcactgccatc
ctgccagaagatgaagcgggttgtgcagcataccaagggttgcaaacggaaaaccaatg
gcgggtgccccatctgcaagcagctcattgccctctgctgctaccatgccaagcactgc
caggagaacaaatgcccggtgccgttctgcctaaacatcaagcagaagctccggcagca
acagctgcagcaccgactacagcaggcccaaatgcttcgcaggaggatggccagcatgc
agcggactggtgtggttgggcagcaacagggcctcccttcccccactcctgccactcca
acgacaccaactggccaacagccaaccaccccgcagacgccccagcccacttctcagcc
tcagcctacccctcccaatagcatgccaccctacttgcccaggactcaagctgctggcc
ctgtgtcccagggtaaggcagcaggccaggtgacccctccaacccctcctcagactgct
cagccaccccttccagggcccccacctgcagcagtggaaatggcaatgcagattcagag
agcagcggagacgcagcgccagatggcccacgtgcaaatttttcaaaggccaatccaac
accagatgcccccgatgactcccatggcccccatgggtatgaacccacctcccatgacc
agaggtcccagtgggcatttggagccagggatgggaccgacagggatgcagcaacagcc
accctggagccaaggaggattgcctcagccccagcaactacagtctgggatgccaaggc
cagccatgatgtcagtggcccagcatggtcaacctttgaacatggctccacaaccagga
ttgggccaggtaggtatcagcccactcaaaccaggcactgtgtctcaacaagccttaca
aaaccttttgcggactctcaggtctcccagctctcccctgcagcagcaacaggtgctta
gtatccttcacgccaacccccagctgttggctgcattcatcaagcagcgggctgccaag
tatgccaactctaatccacaacccatccctgggcagcctggcatgccccaggggcagcc
agggctacagccacctaccatgccaggtcagcagggggtccactccaatccagccatgc
agaacatgaatccaatgcaggcgggcgttcagagggctggcctgccccagcagcaacca
cagcagcaactccagccacccatgggagggatgagcccccaggctcagcagatgaacat
gaaccacaacaccatgccttcacaattccgagacatcttgagacgacagcaaatgatgc
aacagcagcagcaacagggagcagggccaggaataggccctggaatggccaaccataac
cagttccagcaaccccaaggagttggctacccaccacagcagcagcagcggatgcagca
tcacatgcaacagatgcaacaaggaaatatgggacagataggccagcttccccaggcct
tgggagcagaggcaggtgccagtctacaggcctatcagcagcgactccttcagcaacag
atggggtcccctgttcagcccaaccccatgagcccccagcagcatatgctcccaaatca
ggcccagtccccacacctacaaggccagcagatccctaattctctctccaatcaagtgc
gctctccccagcctgtcccttctccacggccacagtcccagcccccccactccagtcct
tccccaaggatgcagcctcagccttctccacaccacgtttccccacagacaagttcccc
acatcctggactggtagctgcccaggccaaccccatggaacaagggcattttgccagcc
cggaccagaattcaatgctttctcagcttgctagcaatccaggcatggcaaacctccat
ggtgcaagcgccacggacctgggactcagcaccgataactcagacttgaattcaaacct
ctcacagagtacactagacatacactagagacaccttgtagtattttgggagcaaaaaa
attattttctcttaacaagactttttgtactgaaaacaatttttttgaatctttcgtag
cctaaaagacaattttccttggaacacataagaactgtgcagtagccgtttgtggttta
aagcaaacatgcaagatgaacctgagggatgatagaatacaaagaatatatttttgtta
tggctggttaccaccagcctttcttcccctttgtgtgtgtggttcaagtgtgcactggg
aggaggctgaggcctgtgaagccaaacaatatgctcctgccttgcacctccaataggtt
ttattattttttttaaattaatgaacatatgtaatattaatagttattatttactggtg
cagatggttgacatttttccctattttcctcactttatggaagagttaaaacatttcta
aaccagaggacaaaaggggttaatgttactttaaaattacattctatatatatataaat
atatataaatatatattaaaataccagttttttttctctgggtgcaaagatgttcattc
ttttaaaaaatgtttaaaaaaaaaaaaaaactgcctttcttcccctcaagtcaactttt
gtgctccagaaaattttctattctgtaagtctgagcgtaaaacttcaagtattaaaata
atttgtacatgtagagagaaaaatgactttttcaaaaatatacaggggcagctgccaaa
ttgatgtattatatattgtggtttctgtttcttgaaagaatttttttcgttatttttac
atctaacaaagtaaaaaaattaaaaagagggtaagaaacgattccggtgggatgatttt
aacatgcaaaatgtccctgggggtttcttctttgcttgctttcttcctccttaccctac
cccccactcacacacacacacacacacacacacacacacacacacacacactttctata
aaacttgaaaatagcaaaaaccctcaactgttgtaaatcatgcaattaaagttgattac
ttataaatatgaactttggatcactgtatagactgttaaatttgatttcttattaccta
ttgttaaataaactgtgtgagacagaca
SEQ ID NO: 7
gagaaggaggaggacagcgccgaggaggaagaggttgatggcggcggcggagctccgag
agacctcggctgggcaggggccggccgtggcgggccggggactgcgcctctagagccgc
gagttctcgggaattcgccgcagcggacgcgctcggcgaatttgtgctcttgtgccctc
ctccgggcttgggcccaggcccggcccctcgcacttgcccttaccttttctatcgagtc
cgcatccctctccagccactgcgacccggcgaagagaaaaaggaacttcccccaccccc
tcgggtgccgtcggagccccccagcccacccctgggtgcggcgcggggaccccgggccg
aagaagagatttcctgaggattctggttttcctcgcttgtatctccgaaagaattaaaa
atggccgagaatgtggtggaaccggggccgccttcagccaagcggcctaaactctcatc
tccggccctctcggcgtccgccagcgatggcacagattttggctctctatttgacttgg
agcacgacttaccagatgaattaatcaactctacagaattgggactaaccaatggtggt
gatattaatcagcttcagacaagtcttggcatggtacaagatgcagcttctaaacataa
acagctgtcagaattgctgcgatctggtagttcccctaacctcaatatgggagttggtg
gcccaggtcaagtcatggccagccaggcccaacagagcagtcctggattaggtttgata
aatagcatggtcaaaagcccaatgacacaggcaggcttgacttctcccaacatggggat
gggcactagtggaccaaatcagggtcctacgcagtcaacaggtatgatgaacagtccag
taaatcagcctgccatgggaatgaacacagggatgaatgcgggcatgaatcctggaatg
ttggctgcaggcaatggacaagggataatgcctaatcaagtcatgaacggttcaattgg
agcaggccgagggcgacagaatatgcagtacccaaacccaggcatgggaagtgctggca
acttactgactgagcctcttcagcagggctctccccagatgggaggacaaacaggattg
agaggcccccagcctcttaagatgggaatgatgaacaaccccaatccttatggttcacc
atatactcagaatcctggacagcagattggagccagtggccttggtctccagattcaga
caaaaactgtactatcaaataacttatctccatttgctatggacaaaaaggcagttcct
ggtggaggaatgcccaacatgggtcaacagccagccccgcaggtccagcagccaggcct
ggtgactccagttgcccaagggatgggttctggagcacatacagctgatccagagaagc
gcaagctcatccagcagcagcttgttctccttttgcatgctcacaagtgccagcgccgg
gaacaggccaatggggaagtgaggcagtgcaaccttccccactgtcgcacaatgaagaa
tgtcctaaaccacatgacacactgccagtcaggcaagtcttgccaagtggcacactgtg
catcttctcgacaaatcatttcacactggaagaattgtacaagacatgattgtcctgtg
tgtctccccctcaaaaatgctggtgataagagaaatcaacagccaattttgactggagc
acccgttggacttggaaatcctagctctctaggggtgggtcaacagtctgcccccaacc
taagcactgttagtcagattgatcccagctccatagaaagagcctatgcagctcttgga
ctaccctatcaagtaaatcagatgccgacacaaccccaggtgcaagcaaagaaccagca
gaatcagcagcctgggcagtctccccaaggcatgcggcccatgagcaacatgagtgcta
gtcctatgggagtaaatggaggtgtaggagttcaaacgccgagtcttctttctgactca
atgttgcattcagccataaattctcaaaacccaatgatgagtgaaaatgccagtgtgcc
ctccctgggtcctatgccaacagcagctcaaccatccactactggaattcggaaacagt
ggcacgaagatattactcaggatcttcgaaatcatcttgttcacaaactcgtccaagcc
atatttcctacgccggatcctgctgctttaaaagacagacggatggaaaacctagttgc
atatgctcggaaagttgaaggggacatgtatgaatctgcaaacaatcgagcggaatact
accaccttctagctgagaaaatctataagatccagaaagaactagaagaaaaacgaagg
accagactacagaagcagaacatgctaccaaatgctgcaggcatggttccagtttccat
gaatccagggcctaacatgggacagccgcaaccaggaatgacttctaatggccctctac
ctgacccaagtatgatccgtggcagtgtgccaaaccagatgatgcctcgaataactcca
caatctggtttgaatcaatttggccagatgagcatggcccagccccctattgtaccccg
gcaaacccctcctcttcagcaccatggacagttggctcaacctggagctctcaacccgc
ctatgggctatgggcctcgtatgcaacagccttccaaccagggccagttccttcctcag
actcagttcccatcacagggaatgaatgtaacaaatatccctttggctccgtccagcgg
tcaagctccagtgtctcaagcacaaatgtctagttcttcctgcccggtgaactctccta
taatgcctccagggtctcaggggagccacattcactgtccccagcttcctcaaccagct
cttcatcagaattcaccctcgcctgtacctagtcgtacccccacccctcaccatactcc
cccaagcataggggctcagcagccaccagcaacaacaattccagcccctgttcctacac
ctcctgccatgccacctgggccacagtcccaggctctacatccccctccaaggcagaca
cctacaccaccaacaacacaacttccccaacaagtgcagccttcacttcctgctgcacc
ttctgctgaccagccccagcagcagcctcgctcacagcagagcacagcagcgtctgttc
ctaccccaacagcaccgctgcttcctccgcagcctgcaactccactttcccagccagct
gtaagcattgaaggacaggtatcaaatcctccatctactagtagcacagaagtgaattc
tcaggccattgctgagaagcagccttcccaggaagtgaagatggaggccaaaatggaag
tggatcaaccagaaccagcagatactcagccggaggatatttcagagtctaaagtggaa
gactgtaaaatggaatctaccgaaacagaagagagaagcactgagttaaaaactgaaat
aaaagaggaggaagaccagccaagtacttcagctacccagtcatctccggctccaggac
agtcaaagaaaaagattttcaaaccagaagaactacgacaggcactgatgccaactttg
gaggcactttaccgtcaggatccagaatcccttccctttcgtcaacctgtggaccctca
gcttttaggaatccctgattactttgatattgtgaagagccccatggatctttctacca
ttaagaggaagttagacactggacagtatcaggagccctggcagtatgtcgatgatatt
tggcttatgttcaataatgcctggttatataaccggaaaacatcacgggtatacaaata
ctgctccaagctctctgaggtctttgaacaagaaattgacccagtgatgcaaagccttg
gatactgttgtggcagaaagttggagttctctccacagacactgtgttgctacggcaaa
cagttgtgcacaatacctcgtgatgccacttattacagttaccagaacaggtatcattt
ctgtgagaagtgtttcaatgagatccaaggggagagcgtttctttgggggatgaccctt
cccagcctcaaactacaataaataaagaacaattttccaagagaaaaaatgacacactg
gatcctgaactgtttgttgaatgtacagagtgcggaagaaagatgcatcagatctgtgt
ccttcaccatgagatcatctggcctgctggattcgtctgtgatggctgtttaaagaaaa
gtgcacgaactaggaaagaaaataagttttctgctaaaaggttgccatctaccagactt
ggcacctttctagagaatcgtgtgaatgactttctgaggcgacagaatcaccctgagtc
aggagaggtcactgttagagtagttcatgcttctgacaaaaccgtggaagtaaaaccag
gcatgaaagcaaggtttgtggacagtggagagatggcagaatcctttccataccgaacc
aaagccctctttgcctttgaagaaattgatggtgttgacctgtgcttctttggcatgca
tgttcaagagtatggctctgactgccctccacccaaccagaggagagtatacatatctt
acctcgatagtgttcatttcttccgtcctaaatgcttgaggactgcagtctatcatgaa
atcctaattggatatttagaatatgtcaagaaattaggttacacaacagggcatatttg
ggcatgtccaccaagtgagggagatgattatatcttccattgccatcctcctgaccaga
agatacccaagcccaagcgactgcaggaatggtacaaaaaaatgcttgacaaggctgta
tcagagcgtattgtccatgactacaaggatatttttaaacaagctactgaagatagatt
aacaagtgcaaaggaattgccttatttcgagggtgatttctggcccaatgttctggaag
aaagcattaaggaactggaacaggaggaagaagagagaaaacgagaggaaaacaccagc
aatgaaagcacagatgtgaccaagggagacagcaaaaatgctaaaaagaagaataataa
gaaaaccagcaaaaataagagcagcctgagtaggggcaacaagaagaaacccgggatgc
ccaatgtatctaacgacctctcacagaaactatatgccaccatggagaagcataaagag
gtcttctttgtgatccgcctcattgctggccctgctgccaactccctgcctcccattgt
tgatcctgatcctctcatcccctgcgatctgatggatggtcgggatgcgtttctcacgc
tggcaagggacaagcacctggagttctcttcactccgaagagcccagtggtccaccatg
tgcatgctggtggagctgcacacgcagagccaggaccgctttgtctacacctgcaatga
atgcaagcaccatgtggagacacgctggcactgtactgtctgtgaggattatgacttgt
gtatcacctgctataacactaaaaaccatgaccacaaaatggagaaactaggccttggc
ttagatgatgagagcaacaaccagcaggctgcagccacccagagcccaggcgattctcg
ccgcctgagtatccagcgctgcatccagtctctggtccatgcttgccagtgtcggaatg
ccaattgctcactgccatcctgccagaagatgaagcgggttgtgcagcataccaagggt
tgcaaacggaaaaccaatggcgggtgccccatctgcaagcagctcattgccctctgctg
ctaccatgccaagcactgccaggagaacaaatgcccggtgccgttctgcctaaacatca
agcagaagctccggcagcaacagctgcagcaccgactacagcaggcccaaatgcttcgc
aggaggatggccagcatgcagcggactggtgtggttgggcagcaacagggcctcccttc
ccccactcctgccactccaacgacaccaactggccaacagccaaccaccccgcagacgc
cccagcccacttctcagcctcagcctacccctcccaatagcatgccaccctacttgccc
aggactcaagctgctggccctgtgtcccagggtaaggcagcaggccaggtgacccctcc
aacccctcctcagactgctcagccaccccttccagggcccccacctgcagcagtggaaa
tggcaatgcagattcagagagcagcggagacgcagcgccagatggcccacgtgcaaatt
tttcaaaggccaatccaacaccagatgcccccgatgactcccatggcccccatgggtat
gaacccacctcccatgaccagaggtcccagtgggcatttggagccagggatgggaccga
cagggatgcagcaacagccaccctggagccaaggaggattgcctcagccccagcaacta
cagtctgggatgccaaggccagccatgatgtcagtggcccagcatggtcaacctttgaa
catggctccacaaccaggattgggccaggtaggtatcagcccactcaaaccaggcactg
tgtctcaacaagccttacaaaaccttttgcggactctcaggtctcccagctctcccctg
cagcagcaacaggtgcttagtatccttcacgccaacccccagctgttggctgcattcat
caagcagcgggctgccaagtatgccaactctaatccacaacccatccctgggcagcctg
gcatgccccaggggcagccagggctacagccacctaccatgccaggtcagcagggggtc
cactccaatccagccatgcagaacatgaatccaatgcaggcgggcgttcagagggctgg
cctgccccagcagcaaccacagcagcaactccagccacccatgggagggatgagccccc
aggctcagcagatgaacatgaaccacaacaccatgccttcacaattccgagacatcttg
agacgacagcaaatgatgcaacagcagcagcaacagggagcagggccaggaataggccc
tggaatggccaaccataaccagttccagcaaccccaaggagttggctacccaccacagc
agcagcagcggatgcagcatcacatgcaacagatgcaacaaggaaatatgggacagata
ggccagcttccccaggccttgggagcagaggcaggtgccagtctacaggcctatcagca
gcgactccttcagcaacagatggggtcccctgttcagcccaaccccatgagcccccagc
agcatatgctcccaaatcaggcccagtccccacacctacaaggccagcagatccctaat
tctctctccaatcaagtgcgctctccccagcctgtcccttctccacggccacagtccca
gcccccccactccagtccttccccaaggatgcagcctcagccttctccacaccacgttt
ccccacagacaagttccccacatcctggactggtagctgcccaggccaaccccatggaa
caagggcattttgccagcccggaccagaattcaatgctttctcagcttgctagcaatcc
aggcatggcaaacctccatggtgcaagcgccacggacctgggactcagcaccgataact
cagacttgaattcaaacctctcacagagtacactagacatacactagagacaccttgta
gtattttgggagcaaaaaaattattttctcttaacaagactttttgtactgaaaacaat
ttttttgaatctttcgtagcctaaaagacaattttccttggaacacataagaactgtgc
agtagccgtttgtggtttaaagcaaacatgcaagatgaacctgagggatgatagaatac
aaagaatatatttttgttatggctggttaccaccagcctttcttcccctttgtgtgtgt
ggttcaagtgtgcactgggaggaggctgaggcctgtgaagccaaacaatatgctcctgc
cttgcacctccaataggttttattattttttttaaattaatgaacatatgtaatattaa
tagttattatttactggtgcagatggttgacatttttccctattttcctcactttatgg
aagagttaaaacatttctaaaccagaggacaaaaggggttaatgttactttaaaattac
attctatatatatataaatatatataaatatatattaaaataccagttttttttctctg
ggtgcaaagatgttcattcttttaaaaaatgtttaaaaaaaaaaaaaaactgcctttct
tcccctcaagtcaacttttgtgctccagaaaattttctattctgtaagtctgagcgtaa
aacttcaagtattaaaataatttgtacatgtagagagaaaaatgactttttcaaaaata
tacaggggcagctgccaaattgatgtattatatattgtggtttctgtttcttgaaagaa
tttttttcgttatttttacatctaacaaagtaaaaaaattaaaaagagggtaagaaacg
attccggtgggatgattttaacatgcaaaatgtccctgggggtttcttctttgcttgct
ttcttcctccttaccctaccccccactcacacacacacacacacacacacacacacaca
cacacacacactttctataaaacttgaaaatagcaaaaaccctcaactgttgtaaatca
tgcaattaaagttgattacttataaatatgaactttggatcactgtatagactgttaaa
tttgatttcttattacctattgttaaataaactgtgtgagacagaca
SEQ ID NO: 8
gccagcgcctgcgcactgagggcggcctggtcgtcgtctgcggcggcggcggcggctga
ggagcccggctgaggcgccagtacccggcccggtccgcatttcgccttccggcttcggt
ttccctcggcccagcacgccccggccccgccccagccctcctgatccctcgcagcccgg
ctccggccgcccgcctctgccgccgcaatgatgatgatggcgctgagcaagaccttcgg
gcagaagcccgtgaagttccagctggaggacgacggcgagttctacatgatcggctccg
aggtgggaaactacctccgtatgttccgaggttctctgtacaagagatacccctcactc
tggaggcgactagccactgtggaagagaggaagaaaatagttgcatcgtcacatggtaa
aaaaacaaaacctaacactaaggatcacggatacacgactctagccaccagtgtgaccc
tgttaaaagcctcggaagtggaagagattctggatggcaacgatgagaagtacaaggct
gtgtccatcagcacagagccccccacctacctcagggaacagaaggccaagaggaacag
ccagtgggtacccaccctgcccaacagctcccaccacttagatgccgtgccatgctcca
caaccatcaacaggaaccgcatgggccgagacaagaagagaaccttccccctttgcttt
gatgaccatgacccagctgtgatccatgagaacgcatctcagcccgaggtgctggtccc
catccggctggacatggagatcgatgggcagaagctgcgagacgccttcacctggaaca
tgaatgagaagttgatgacgcctgagatgttttcagaaatcctctgtgacgatctggat
ttgaacccgctgacgtttgtgccagccatcgcctctgccatcagacagcagatcgagtc
ctaccccacggacagcatcctggaggaccagtcagaccagcgcgtcatcatcaagctga
acatccatgtgggaaacatttccctggtggaccagtttgagtgggacatgtcagagaag
gagaactcaccagagaagtttgccctgaagctgtgctcggagctggggttgggcgggga
gtttgtcaccaccatcgcatacagcatccggggacagctgagctggcatcagaagacct
acgccttcagcgagaaccctctgcccacagtggagattgccatccggaacacgggcgat
gcggaccagtggtgcccactgctggagactctgacagacgctgagatggagaagaagat
ccgcgaccaggacaggaacacgaggcggatgaggcgtcttgccaacacggccccggcct
ggtaaccagcccatcagcacacggctcccacggagcatctcagaagattgggccgcctc
tcctccatcttctggcaaggacagaggcgaggggacagcccagcgccatcctgaggatc
gggtgggggtggagtgggggcttccaggtggcccttcccggcacacattccatttgttg
agccccagtcctgccccccaccccaccctccctacccctccccagtctctggggtcagg
aagaaaccttattttaggttgtgttttgtttttgtataggagccccaggcagggctagt
aacagtttttaaataaaaggcaacaggtcatgttcaatttcttcaacaggtcatgttca
atttcttcaaagttttaacataaaaataatgagagccaggagtggggccggggcctggg
gggacgaaggtggtatgtgaacaaggttggcacacaggcctcaccctcctctgcctcag
attcccaagtgggcaggtgggggtgaatggggctccgggtagcacctcagctcctctca
gctcccctcagcctgttctccttccagacccagagagctgagaagagtagctgtgaggc
tcagggcaagaggctctctgcctttcaggaacagccctaaccctgctccccttgcttgg
cctcaggaaggtgccgcgagctctcctgccgtccctgggccgccctggctctgctgtgt
ccagatggtcaggctactgccagctggggccttgctgctctgaagtcccctgcggaggg
cccagtcctgtgtgggcactgctgggctgtcgccagcctgggtgcaggagggctgttct
agctccagtggcacccatagccaggtcagctggggccctttcccaccccagcaggtgct
gtggcctgggccagctcctgccttacaagccagctgtgaggaatatgggaatagccctc
ccggcctggtgccagctcttggagttgacacggtacagggaggagacacagcccagggt
cccttcccagccctgcctccaaggagttcatgtcccctctgttctcatctgtaataggg
aggtgtccccattcttcagaatggacacaggatctgggagggcagcaaactggctcgca
gctccagccttactgaagagaatgggcacagatccgggcacagatcccagcacagactg
ctgccaccctcagctgttggcaggtcccatgctgccagggcagggctagggtcagaggc
tgctgtgctccctggaagtggggtagggccccatgtggggcagaggcagagctctgatt
agggattggggttcttggtcgctgagatgtgagaggagggctcctttgagcacatgtta
gcatgggactcttcccagggagtttgcactcagggcctctgccctccatcaaagagtgg
aactccccagagccccatgcacagcaaggggacagctgggccttactggaaggccttga
acaaaggggaagattcccagcccagctgctcttagacatgaacaggtttcattgctgag
gtgtttgttctgtccatgaggtaggaacctcggcaatgaaagggtgaggcagccctgtg
tctccacaactggggggatggaaggaaccttggctgcctcaccccacaggtcgggcagg
gccacctggctgggaggtgccgggaaggctgggccctcactcctgaccgccagctcaca
ccgccgcaaagccatctccacaaggtctggctacaacacggagggcagactcaacagag
aacagtgttgttaccatgaaaatgacaacctgtctttggaggaggccccgtgccactga
gcatccagaaataaaccacaacatggacaggcttagaacaacaaggaaagctgccaggt
cagaagagaaaaatgagccacaggggtcggataaggctcacacacgtcctcagctaaaa
agggcaggaacagaaccttccagaagtccctgcctcacccagtctcagaactctgctaa
ggtgaaaacttaggctctgaggtcatagaaagggcagaagacctagtcctggccctctt
ctgcacctgaatccatggggctttggcatcaccagatgaaaaatgaggcatacgcccac
ctgtcagggtggctgatgagagacaggagaggctagattggcatcagcctgaaggcacc
actggcaggaacatctgtaggctggtttggcacaacctaggagacgcctgtcctggccc
cagcagccgaaatctggtgaacttccccgctgactggcaggtagcagaggcctatggtg
ggcaggacttgcccaaggccctggtggggccaggatgagaaccctgagcctgtcacctg
tgagctcaaaagctctgcctggcaacctgtgagctcaaagctctgccaggcaaccatgg
gcagtttctttgccctctgtgggcacccctatcctaccacctgcagttgggctgagagg
ccacactgagtgaggacggggcaggcatagaaggatgtggccaggtgagatggggaagc
cagtgctgtgggccaagagactgcagctcattctgtttattcaggtgggcccttgcatg
ggcccagcctttaggatgggttttttctgccccaagtaggggtcatgggtaggatggaa
gctgccagaagcctcttaggcctggccctgggtgggggtcactgctgcgggggtggcag
atggggtcctggctgttcctcagggaggggcaggtaattggggtcttctgcaggggcat
ccaggagcagctttctgtggggaggggcccgtgttgagcacaggccagcacaggtcccc
atcggtggggatccttctgagggtggggagagggagggagggctctcaacactcacagg
aagccaggggtctgcaggagcctcttgcctccaggctggttggggaagacgtcctccag
gaagtagtagatatggcccaccgcaatccctgtgagacagccacggactgtggggtcac
cctccacagcccagagtcctagaccagcagagcctgccccaggcccccatccacagcct
ggtggccctgcaggccccacagcatgagtgccccaaagccttgcacagagtgccagccc
cgggttggccgtgaaggacaagcttaaaaggcccagaagcaggcaggacccagggaggg
gagggcctgagaatagtggaggagtgggagccatggggcaggaaccctgaccctcccat
cctcactcccatcaggaccgtgcaagcatcagtagatccgtcctgacgatgcaaattat
gtgggccggctggcttgaggggctgtaagagcacagcagctgggagggcaggaagatgg
ggatggagccaggtgtgaggagaactccagcaaggatgggagaggggccccagggcata
agcagcgtgtcctgaggggagtggccagcctggggcggactagatgtaccgggaggctc
acccagcaggtccacgaggatggagttgcccagcagcagcgagaagcccatgagcgccc
aaggcaggaacggtgcctggaaagtgagcaggccgaagaagttgaccctcacccgaggg
ctgcggcggctccacacgtacaccagcatggccatgagggcctggcccaggaagaacag
gctgcccaggagtcccagcagctgggccagagtcaaggtgctccggtgcaggcctcagc
ccaagcccagggcccctctgacttcccaagaccctggaattcttcccctcatctcccct
atgtgctattccctcatcaagatgagccagtccaataaaggcgacacactccacgggc
SEQ ID NO: 9
gccagcgcctgcgcactgagggcggcctggtcgtcgtctgcggcggcggcggcggctga
ggagcccggctgaggcgccagtacccggcccggtccgcatttcgccttccggcttcggt
ttccctcggcccagcacgccccggccccgccccagccctcctgatccctcgcagcccgg
ctccggccgcccgcctctgccgccgcaatgatgatgatggcgctgagcaagaccttcgg
gcagaagcccgtgaagttccagctggaggacgacggcgagttctacatgatcggctccg
aggtgggaaactacctccgtatgttccgaggttctctgtacaagagatacccctcactc
tggaggcgactagccactgtggaagagaggaagaaaatagttgcatcgtcacatgatca
cggatacacgactctagccaccagtgtgaccctgttaaaagcctcggaagtggaagaga
ttctggatggcaacgatgagaagtacaaggctgtgtccatcagcacagagccccccacc
tacctcagggaacagaaggccaagaggaacagccagtgggtacccaccctgcccaacag
ctcccaccacttagatgccgtgccatgctccacaaccatcaacaggaaccgcatgggcc
gagacaagaagagaaccttccccctttgctttgatgaccatgacccagctgtgatccat
gagaacgcatctcagcccgaggtgctggtccccatccggctggacatggagatcgatgg
gcagaagctgcgagacgccttcacctggaacatgaatgagaagttgatgacgcctgaga
tgttttcagaaatcctctgtgacgatctggatttgaacccgctgacgtttgtgccagcc
atcgcctctgccatcagacagcagatcgagtcctaccccacggacagcatcctggagga
ccagtcagaccagcgcgtcatcatcaagctgaacatccatgtgggaaacatttccctgg
tggaccagtttgagtgggacatgtcagagaaggagaactcaccagagaagtttgccctg
aagctgtgctcggagctggggttgggcggggagtttgtcaccaccatcgcatacagcat
ccggggacagctgagctggcatcagaagacctacgccttcagcgagaaccctctgccca
cagtggagattgccatccggaacacgggcgatgcggaccagtggtgcccactgctggag
actctgacagacgctgagatggagaagaagatccgcgaccaggacaggaacacgaggcg
gatgaggcgtcttgccaacacggccccggcctggtaaccagcccatcagcacacggctc
ccacggagcatctcagaagattgggccgcctctcctccatcttctggcaaggacagagg
cgaggggacagcccagcgccatcctgaggatcgggtgggggtggagtgggggcttccag
gtggcccttcccggcacacattccatttgttgagccccagtcctgccccccaccccacc
ctccctacccctccccagtctctggggtcaggaagaaaccttattttaggttgtgtttt
gtttttgtataggagccccaggcagggctagtaacagtttttaaataaaaggcaacagg
tcatgttcaatttcttcaacaggtcatgttcaatttcttcaaagttttaacataaaaat
aatgagagccaggagtggggccggggcctggggggacgaaggtggtatgtgaacaaggt
tggcacacaggcctcaccctcctctgcctcagattcccaagtgggcaggtgggggtgaa
tggggctccgggtagcacctcagctcctctcagctcccctcagcctgttctccttccag
acccagagagctgagaagagtagctgtgaggctcagggcaagaggctctctgcctttca
ggaacagccctaaccctgctccccttgcttggcctcaggaaggtgccgcgagctctcct
gccgtccctgggccgccctggctctgctgtgtccagatggtcaggctactgccagctgg
ggccttgctgctctgaagtcccctgcggagggcccagtcctgtgtgggcactgctgggc
tgtcgccagcctgggtgcaggagggctgttctagctccagtggcacccatagccaggtc
agctggggccctttcccaccccagcaggtgctgtggcctgggccagctcctgccttaca
agccagctgtgaggaatatgggaatagccctcccggcctggtgccagctcttggagttg
acacggtacagggaggagacacagcccagggtcccttcccagccctgcctccaaggagt
tcatgtcccctctgttctcatctgtaatagggaggtgtccccattcttcagaatggaca
caggatctgggagggcagcaaactggctcgcagctccagccttactgaagagaatgggc
acagatccgggcacagatcccagcacagactgctgccaccctcagctgttggcaggtcc
catgctgccagggcagggctagggtcagaggctgctgtgctccctggaagtggggtagg
gccccatgtggggcagaggcagagctctgattagggattggggttcttggtcgctgaga
tgtgagaggagggctcctttgagcacatgttagcatgggactcttcccagggagtttgc
actcagggcctctgccctccatcaaagagtggaactccccagagccccatgcacagcaa
ggggacagctgggccttactggaaggccttgaacaaaggggaagattcccagcccagct
gctcttagacatgaacaggtttcattgctgaggtgtttgttctgtccatgaggtaggaa
cctcggcaatgaaagggtgaggcagccctgtgtctccacaactggggggatggaaggaa
ccttggctgcctcaccccacaggtcgggcagggccacctggctgggaggtgccgggaag
gctgggccctcactcctgaccgccagctcacaccgccgcaaagccatctccacaaggtc
tggctacaacacggagggcagactcaacagagaacagtgttgttaccatgaaaatgaca
acctgtctttggaggaggccccgtgccactgagcatccagaaataaaccacaacatgga
caggcttagaacaacaaggaaagctgccaggtcagaagagaaaaatgagccacaggggt
cggataaggctcacacacgtcctcagctaaaaagggcaggaacagaaccttccagaagt
ccctgcctcacccagtctcagaactctgctaaggtgaaaacttaggctctgaggtcata
gaaagggcagaagacctagtcctggccctcttctgcacctgaatccatggggctttggc
atcaccagatgaaaaatgaggcatacgcccacctgtcagggtggctgatgagagacagg
agaggctagattggcatcagcctgaaggcaccactggcaggaacatctgtaggctggtt
tggcacaacctaggagacgcctgtcctggccccagcagccgaaatctggtgaacttccc
cgctgactggcaggtagcagaggcctatggtgggcaggacttgcccaaggccctggtgg
ggccaggatgagaaccctgagcctgtcacctgtgagctcaaaagctctgcctggcaacc
tgtgagctcaaagctctgccaggcaaccatgggcagtttctttgccctctgtgggcacc
cctatcctaccacctgcagttgggctgagaggccacactgagtgaggacggggcaggca
tagaaggatgtggccaggtgagatggggaagccagtgctgtgggccaagagactgcagc
tcattctgtttattcaggtgggcccttgcatgggcccagcctttaggatgggttttttc
tgccccaagtaggggtcatgggtaggatggaagctgccagaagcctcttaggcctggcc
ctgggtgggggtcactgctgcgggggtggcagatggggtcctggctgttcctcagggag
gggcaggtaattggggtcttctgcaggggcatccaggagcagctttctgtggggagggg
cccgtgttgagcacaggccagcacaggtccccatcggtggggatccttctgagggtggg
gagagggagggagggctctcaacactcacaggaagccaggggtctgcaggagcctcttg
cctccaggctggttggggaagacgtcctccaggaagtagtagatatggcccaccgcaat
ccctgtgagacagccacggactgtggggtcaccctccacagcccagagtcctagaccag
cagagcctgccccaggcccccatccacagcctggtggccctgcaggccccacagcatga
gtgccccaaagccttgcacagagtgccagccccgggttggccgtgaaggacaagcttaa
aaggcccagaagcaggcaggacccagggaggggagggcctgagaatagtggaggagtgg
gagccatggggcaggaaccctgaccctcccatcctcactcccatcaggaccgtgcaagc
atcagtagatccgtcctgacgatgcaaattatgtgggccggctggcttgaggggctgta
agagcacagcagctgggagggcaggaagatggggatggagccaggtgtgaggagaactc
cagcaaggatgggagaggggccccagggcataagcagcgtgtcctgaggggagtggcca
gcctggggcggactagatgtaccgggaggctcacccagcaggtccacgaggatggagtt
gcccagcagcagcgagaagcccatgagcgcccaaggcaggaacggtgcctggaaagtga
gcaggccgaagaagttgaccctcacccgagggctgcggcggctccacacgtacaccagc
atggccatgagggcctggcccaggaagaacaggctgcccaggagtcccagcagctgggc
cagagtcaaggtgctccggtgcaggcctcagcccaagcccagggcccctctgacttccc
aagaccctggaattcttcccctcatctcccctatgtgctattccctcatcaagatgagc
cagtccaataaaggcgacacactccacgggc
SEQ ID NO: 10
gtactctgggtgactcagagagggaagagattcagccagcacactcctcgcgagcaagc
attactctactgactggcagagacaggagaggtagatgtccacgcccacagaccctggt
gcgatgccccacccagggccttcgccggggcctgggccttcccctgggccaattcttgg
gcctagtccaggaccaggaccatccccaggttccgtccacagcatgatggggccaagtc
ctggacctccaagtgtctcccatcctatgccgacgatggggtccacagacttcccacag
gaaggcatgcatcaaatgcataagcccatcgatggtatacatgacaaggggattgtaga
agacatccattgtggatccatgaagggcactggtatgcgaccacctcacccaggcatgg
gccctcc
ccagagtccaatggatcaacacagccaaggttatatgtcaccacacccatctccattag
gagccccagagcacgtctccagccctatgtctggaggaggcccaactccacctcagatg
ccaccaagccagccgggggccctcatcccaggtgatccgcaggccatgagccagcccaa
cagaggtccctcacctttcagtcctgtccagctgcatcagcttcgagctcagattttag
cttataaaatgctggcccgaggccagcccctccccgaaacgctgcagcttgcagtccag
gggaaaaggacgttgcctggcttgcagcaacaacagcagcagcaacagcagcagcagca
gcagcagcagcagcagcagcagcagcaacagcagccgcagcagcagccgccgcaaccac
agacgca
gcaacaacagcagccggcccttgttaactacaacagaccatctggcccggggccggagc
tgagcggcccgagcaccccgcagaagctgccggtgcccgcgcccggcggccggccctcg
cccgcgccccccgcagccgcgcagccgcccgcggccgcagtgcccgggccctcagtgcc
gcagccggccccggggcagccctcgcccgtcctccagctgcagcagaagcagagccgca
tcagccccatccagaaaccgcaaggcctggaccccgtggaaattctgcaagagcgggaa
tacagacttcaggcccgcatagctcataggatacaagaactggaaaatctgcctggctc
tttgccaccagatttaagaaccaaagcaaccgtggaactaaaagcacttcggttactca
atttcca
gcgtcagctgagacaggaggtggtggcctgcatgcgcagggacacgaccctggagacgg
ctctcaactccaaagcatacaaacggagcaagcgccagactctgagagaagctcgcatg
accgagaagctggagaagcagcagaagattgagcaggagaggaaacgccgtcagaaaca
ccaggaatacctgaacagtattttgcaacatgcaaaagattttaaggaatatcatcggt
ctgtggccggaaagatccagaagctctccaaagcagtggcaacttggcatgccaacact
gaaagagagcagaagaaggagacagagcggattgaaaaggagagaatgcggcgactgat
ggctgaagatgaggagggttatagaaaactgattgatcaaaagaaagacaggcgtttag
cttacct
tttgcagcagaccgatgagtatgtagccaatctgaccaatctggtttgggagcacaagc
aagcccaggcagccaaagagaagaagaagaggaggaggaggaagaagaaggctgaggag
aatgcagagggtggggagtctgccctgggaccggatggagagcccatagatgagagcag
ccagatgagtgacctccctgtcaaagtgactcacacagaaaccggcaaggttctgttcg
gaccagaagcacccaaagcaagtcagctggacgcctggctggaaatgaatcctggttat
gaagttgcccctagatctgacagtgaagagagtgattctgattatgaggaagaggatga
ggaagaagagtccagtaggcaggaaaccgaagagaaaatactcctggatccaaatagcg
aagaagt
ttctgagaaggatgctaagcagatcattgagacagctaagcaagacgtggatgatgaat
acagcatgcagtacagtgccaggggctcccagtcctactacaccgtggctcatgccatc
tcggagagggtggagaaacagtctgccctcctaattaatgggaccctaaagcattacca
gctccagggcctggaatggatggtttccctgtataataacaacttgaacggaatcttag
ccgatgaaatggggcttggaaagaccatacagaccattgcactcatcacttatctgatg
gagcacaaaagactcaatggcccctatctcatcattgttcccctttcgactctatctaa
ctggacatatgaatttgacaaatgggctccttctgtggtgaagatttcttacaagggta
ctcctgc
catgcgtcgctcccttgtcccccagctacggagtggcaaattcaatgtcctcttgacta
cttatgagtatattataaaagacaagcacattcttgcaaagattcggtggaaatacatg
atagtggacgaaggccaccgaatgaagaatcaccactgcaagctgactcaggtcttgaa
cactcactatgtggcccccagaaggatcctcttgactgggaccccgctgcagaataagc
tccctgaactctgggccctcctcaacttcctcctcccaacaatttttaagagctgcagc
acatttgaacaatggttcaatgctccatttgccatgactggtgaaagggtggacttaaa
tgaagaagaaactatattgatcatcaggcgtctacataaggtgttaagaccatttttac
taaggagactgaagaaagaagttgaatcccagcttcccgaaaaagtggaatatgtgatc
aagtgtgacatgtcagctctgcagaagattctgtatcgccatatgcaagccaaggggat
ccttctcacagatggttctgagaaagataagaaggggaaaggaggtgctaagacactta
tgaacactattatgcagttgagaaaaatctgcaaccacccatatatgtttcagcacatt
gaggaatcctttgctgaacacctaggctattcaaatggggtcatcaatggggctgaact
gtatcgggcctcagggaagtttgagctgcttgatcgtattctgccaaaattgagagcga
ctaatcaccgagtgctgcttttctgccagatgacatctctcatgaccatcatggaggat
tattttgcttttcg
gaacttcctttacctacgccttgatggcaccaccaagtctgaagatcgtgctgctttgc
tgaagaaattcaatgaacctggatcccagtatttcattttcttgctgagcacaagagct
ggtggcctgggcttaaatcttcaggcagctgatacagtggtcatctttgacagcgactg
gaatcctcatcaggatctgcaggcccaagaccgagctcaccgcatcgggcagcagaacg
aggtccgggtactgaggctctgtaccgtgaacagcgtggaggaaaagatcctcgcggcc
gcaaaatacaagctgaacgtggatcagaaagtgatccaggcgggcatgtttgaccaaaa
gtcttcaagccacgagcggagggcattcctgcaggccatcttggagcatgaggaggaaa
atgagga
agaagatgaagtaccggacgatgagactctgaaccaaatgattgctcgacgagaagaag
aatttgacctttttatgcggatggacatggaccggcggagggaagatgcccggaacccg
aaacggaagccccgtttaatggaggaggatgagctgccctcctggatcattaaggatga
cgctgaagtagaaaggctcacctgtgaagaagaggaggagaaaatatttgggagggggt
cccgccagcgccgtgacgtggactacagtgacgccctcacggagaagcagtggctaagg
gccatcgaagacggcaatttggaggaaatggaagaggaagtacggcttaagaagcgaaa
aagacgaagaaatgtggataaagatcctgcaaaagaagatgtggaaaaagctaagaaga
gaagagg
ccgccctcccgctgagaaactgtcaccaaatccccccaaactgacaaagcagatgaacg
ctatcatcgatactgtgataaactacaaagataggtgtaacgtggagaaggtgcccagt
aattctcagttggaaatagaaggaaacagttcagggcgacagctcagtgaagtcttcat
tcagttaccttcaaggaaagaattaccagaatactatgaattaattaggaagccagtgg
atttcaaaaaaataaaggaaaggattcgtaatcataagtaccggagcctaggcgacctg
gagaaggatgtcatgcttctctgtcacaacgctcagacgttcaacctggagggatccca
gatctatgaagactccatcgtcttacagtcagtgtttaagagtgcccggcagaaaattg
ccaaaga
ggaagagagtgaggatgaaagcaatgaagaggaggaagaggaagatgaagaagagtcag
agtccgaggcaaaatcagtcaaggtgaaaattaagctcaataaaaaagatgacaaaggc
cgggacaaagggaaaggcaagaaaaggccaaatcgaggaaaagccaaacctgtagtgag
cgattttgacagcgatgaggagcaggatgaacgtgaacagtcagaaggaagtgggacgg
atgatgagtgatcagtatggacctttttccttggtagaactgaattccttcctcccctg
tctcatttctacccagtgagttcatttgtcatataggcactgggttgtttctatatcat
catcgtctataaactagctttaggatagtgccagacaaacatatgatatcatggtgtaa
aaaacac
acacatacacaaatatttgtaacatattgtgaccaaatgggcctcaaagattcagattg
aaacaaacaaaaagcttttgatggaaaatatgtgggtggatagtatatttctatgggtg
ggtctaatttggtaacggtttgattgtgcctggttttatcacctgttcagatgagaaga
tttttgtcttttgtagcactgataaccaggagaagccattaaaagccactggttatttt
atttttcatcaggcaattttcgaggtttttatttgttcggtattgtttttttacactgt
ggtacatataagcaactttaataggtgataaatgtacagtagttagatttcacctgcat
atacatttttccattttatgctctatgatctgaacaaaagctttttgaattgtataaga
tttatgt
ctactgtaaacattgcttaatttttttgctcttgatttaaaaaaaagttttgttgaaag
cgctattgaatattgcaatctatatagtgtattggatggcttcttttgtcaccctgatc
tcctatgttaccaatgtgtatcgtctccttctccctaaagtgtacttaatctttgcttt
ctttgcacaatgtctttggttgcaagtcataagcctgaggcaaataaaattccagtaat
ttcgaagaatgtggtgttggtgctttcctaataaagaaataatttagcttga
[SMARCA2 sequence-2]
SEQ ID NO: 11
gtactctgggtgactcagagagggaagagattcagccagcacactcctcgcgagcaagc
attactctactgactggcagagacaggagaggtagatgtccacgcccacagaccctggt
gcgatgccccacccagggccttcgccggggcctgggccttcccctgggccaattcttgg
gcctagtccaggaccaggaccatccccaggttccgtccacagcatgatggggccaagtc
ctggacctccaagtgtctcccatcctatgccgacgatggggtccacagacttcccacag
gaaggcatgcatcaaatgcataagcccatcgatggtatacatgacaaggggattgtaga
agacatccattgtggatccatgaagggcactggtatgcgaccacctcacccaggcatgg
gccctcc
ccagagtccaatggatcaacacagccaaggttatatgtcaccacacccatctccattag
gagccccagagcacgtctccagccctatgtctggaggaggcccaactccacctcagatg
ccaccaagccagccgggggccctcatcccaggtgatccgcaggccatgagccagcccaa
cagaggtccctcacctttcagtcctgtccagctgcatcagcttcgagctcagattttag
cttataaaatgctggcccgaggccagcccctccccgaaacgctgcagcttgcagtccag
gggaaaaggacgttgcctggcttgcagcaacaacagcagcagcaacagcagcagcagca
gcagcagcagcagcagcagcagcagcaacagcagccgcagcagcagccgccgcaaccac
agacgca
gcaacaacagcagccggcccttgttaactacaacagaccatctggcccggggccggagc
tgagcggcccgagcaccccgcagaagctgccggtgcccgcgcccggcggccggccctcg
cccgcgccccccgcagccgcgcagccgcccgcggccgcagtgcccgggccctcagtgcc
gcagccggccccggggcagccctcgcccgtcctccagctgcagcagaagcagagccgca
tcagccccatccagaaaccgcaaggcctggaccccgtggaaattctgcaagagcgggaa
tacagacttcaggcccgcatagctcataggatacaagaactggaaaatctgcctggctc
tttgccaccagatttaagaaccaaagcaaccgtggaactaaaagcacttcggttactca
atttcca
gcgtcagctgagacaggaggtggtggcctgcatgcgcagggacacgaccctggagacgg
ctctcaactccaaagcatacaaacggagcaagcgccagactctgagagaagctcgcatg
accgagaagctggagaagcagcagaagattgagcaggagaggaaacgccgtcagaaaca
ccaggaatacctgaacagtattttgcaacatgcaaaagattttaaggaatatcatcggt
ctgtggccggaaagatccagaagctctccaaagcagtggcaacttggcatgccaacact
gaaagagagcagaagaaggagacagagcggattgaaaaggagagaatgcggcgactgat
ggctgaagatgaggagggttatagaaaactgattgatcaaaagaaagacaggcgtttag
cttacct
tttgcagcagaccgatgagtatgtagccaatctgaccaatctggtttgggagcacaagc
aagcccaggcagccaaagagaagaagaagaggaggaggaggaagaagaaggctgaggag
aatgcagagggtggggagtctgccctgggaccggatggagagcccatagatgagagcag
ccagatgagtgacctccctgtcaaagtgactcacacagaaaccggcaaggttctgttcg
gaccagaagcacccaaagcaagtcagctggacgcctggctggaaatgaatcctggttat
gaagttgcccctagatctgacagtgaagagagtgattctgattatgaggaagaggatga
ggaagaagagtccagtaggcaggaaaccgaagagaaaatactcctggatccaaatagcg
aagaagt
ttctgagaaggatgctaagcagatcattgagacagctaagcaagacgtggatgatgaat
acagcatgcagtacagtgccaggggctcccagtcctactacaccgtggctcatgccatc
tcggagagggtggagaaacagtctgccctcctaattaatgggaccctaaagcattacca
gctccagggcctggaatggatggtttccctgtataataacaacttgaacggaatcttag
ccgatgaaatggggcttggaaagaccatacagaccattgcactcatcacttatctgatg
gagcacaaaagactcaatggcccctatctcatcattgttcccctttcgactctatctaa
ctggacatatgaatttgacaaatgggctccttctgtggtgaagatttcttacaagggta
ctcctgc
catgcgtcgctcccttgtcccccagctacggagtggcaaattcaatgtcctcttgacta
cttatgagtatattataaaagacaagcacattcttgcaaagattcggtggaaatacatg
atagtggacgaaggccaccgaatgaagaatcaccactgcaagctgactcaggtcttgaa
cactcactatgtggcccccagaaggatcctcttgactgggaccccgctgcagaataagc
tccctgaactctgggccctcctcaacttcctcctcccaacaatttttaagagctgcagc
acatttgaacaatggttcaatgctccatttgccatgactggtgaaagggtggacttaaa
tgaagaagaaactatattgatcatcaggcgtctacataaggtgttaagaccatttttac
taaggag
actgaagaaagaagttgaatcccagcttcccgaaaaagtggaatatgtgatcaagtgtg
acatgtcagctctgcagaagattctgtatcgccatatgcaagccaaggggatccttctc
acagatggttctgagaaagataagaaggggaaaggaggtgctaagacacttatgaacac
tattatgcagttgagaaaaatctgcaaccacccatatatgtttcagcacattgaggaat
cctttgctgaacacctaggctattcaaatggggtcatcaatggggctgaactgtatcgg
gcctcagggaagtttgagctgcttgatcgtattctgccaaaattgagagcgactaatca
ccgagtgctgcttttctgccagatgacatctctcatgaccatcatggaggattattttg
cttttcg
gaacttcctttacctacgccttgatggcaccaccaagtctgaagatcgtgctgctttgc
tgaagaaattcaatgaacctggatcccagtatttcattttcttgctgagcacaagagct
ggtggcctgggcttaaatcttcaggcagctgatacagtggtcatctttgacagcgactg
gaatcctcatcaggatctgcaggcccaagaccgagctcaccgcatcgggcagcagaacg
aggtccgggtactgaggctctgtaccgtgaacagcgtggaggaaaagatcctcgcggcc
gcaaaatacaagctgaacgtggatcagaaagtgatccaggcgggcatgtttgaccaaaa
gtcttcaagccacgagcggagggcattcctgcaggccatcttggagcatgaggaggaaa
atgagga
agaagatgaagtaccggacgatgagactctgaaccaaatgattgctcgacgagaagaag
aatttgacctttttatgcggatggacatggaccggcggagggaagatgcccggaacccg
aaacggaagccccgtttaatggaggaggatgagctgccctcctggatcattaaggatga
cgctgaagtagaaaggctcacctgtgaagaagaggaggagaaaatatttgggagggggt
cccgccagcgccgtgacgtggactacagtgacgccctcacggagaagcagtggctaagg
gccatcgaagacggcaatttggaggaaatggaagaggaagtacggcttaagaagcgaaa
aagacgaagaaatgtggataaagatcctgcaaaagaagatgtggaaaaagctaagaaga
gaagagg
ccgccctcccgctgagaaactgtcaccaaatccccccaaactgacaaagcagatgaacg
ctatcatcgatactgtgataaactacaaagatagttcagggcgacagctcagtgaagtc
ttcattcagttaccttcaaggaaagaattaccagaatactatgaattaattaggaagcc
agtggatttcaaaaaaataaaggaaaggattcgtaatcataagtaccggagcctaggcg
acctggagaaggatgtcatgcttctctgtcacaacgctcagacgttcaacctggaggga
tcccagatctatgaagactccatcgtcttacagtcagtgtttaagagtgcccggcagaa
aattgccaaagaggaagagagtgaggatgaaagcaatgaagaggaggaagaggaagatg
aagaaga
gtcagagtccgaggcaaaatcagtcaaggtgaaaattaagctcaataaaaaagatgaca
aaggccgggacaaagggaaaggcaagaaaaggccaaatcgaggaaaagccaaacctgta
gtgagcgattttgacagcgatgaggagcaggatgaacgtgaacagtcagaaggaagtgg
gacggatgatgagtgatcagtatggacctttttccttggtagaactgaattccttcctc
ccctgtctcatttctacccagtgagttcatttgtcatataggcactgggttgtttctat
atcatcatcgtctataaactagctttaggatagtgccagacaaacatatgatatcatgg
tgtaaaaaacacacacatacacaaatatttgtaacatattgtgaccaaatgggcctcaa
agattca
gattgaaacaaacaaaaagcttttgatggaaaatatgtgggtggatagtatatttctat
gggtgggtctaatttggtaacggtttgattgtgcctggttttatcacctgttcagatga
gaagatttttgtcttttgtagcactgataaccaggagaagccattaaaagccactggtt
attttatttttcatcaggcaattttcgaggtttttatttgttcggtattgtttttttac
actgtggtacatataagcaactttaataggtgataaatgtacagtagttagatttcacc
tgcatatacatttttccattttatgctctatgatctgaacaaaagctttttgaattgta
taagatttatgtctactgtaaacattgcttaatttttttgctcttgatttaaaaaaaag
ttttgtt
gaaagcgctattgaatattgcaatctatatagtgtattggatggcttcttttgtcaccc
tgatctcctatgttaccaatgtgtatcgtctccttctccctaaagtgtacttaatcttt
gctttctttgcacaatgtctttggttgcaagtcataagcctgaggcaaataaaattcca
gtaatttcgaagaatgtggtgttggtgctttcctaataaagaaataatttagcttga
[SMARCA4 sequence-1]
SEQ ID NO: 12
gggcgcgcgcgcgaggcttcccctcgtttggcggcggcggcggcttctttgtttcgtga
agagaagcgagacgcccattctgcccccggccccgcgcggaggggcgggggaggcgccg
ggaagtcgacggcgccggcggctcctgcaggaggccactgtctgcagctcccgtgaaga
tgtccactccagacccacccctgggcggaactcctcggccaggtccttccccgggccct
ggcccttcccctggagccatgctgggccctagcccgggtccctcgccgggctccgccca
cagcatgatggggcccagcccagggccgccctcagcaggacaccccatccccacccagg
ggcctggagggtaccctcaggacaacatgcaccagatgcacaagcccatggagtccatg
catgaga
agggcatgtcggacgacccgcgctacaaccagatgaaaggaatggggatgcggtcaggg
ggccatgctgggatggggcccccgcccagccccatggaccagcactcccaaggttaccc
ctcgcccctgggtggctctgagcatgcctctagtccagttccagccagtggcccgtctt
cggggccccagatgtcttccgggccaggaggtgccccgctggatggtgctgacccccag
gccttggggcagcagaaccggggcccaaccccatttaaccagaaccagctgcaccagct
cagagctcagatcatggcctacaagatgctggccagggggcagcccctccccgaccacc
tgcagatggcggtgcagggcaagcggccgatgcccgggatgcagcagcagatgccaacg
ctacctc
caccctcggtgtccgcaacaggacccggccctggccctggccctggccccggcccgggt
cccggcccggcacctccaaattacagcaggcctcatggtatgggagggcccaacatgcc
tcccccaggaccctcgggcgtgccccccgggatgccaggccagcctcctggagggcctc
ccaagccctggcctgaaggacccatggcgaatgctgctgcccccacgagcacccctcag
aagctgattcccccgcagccaacgggccgcccttcccccgcgccccctgccgtcccacc
cgccgcctcgcccgtgatgccaccgcagacccagtcccccgggcagccggcccagcccg
cgcccatggtgccactgcaccagaagcagagccgcatcacccccatccagaagccgcgg
ggcctcg
accctgtggagatcctgcaggagcgcgagtacaggctgcaggctcgcatcgcacaccga
attcaggaacttgaaaaccttcccgggtccctggccggggatttgcgaaccaaagcgac
cattgagctcaaggccctcaggctgctgaacttccagaggcagctgcgccaggaggtgg
tggtgtgcatgcggagggacacagcgctggagacagccctcaatgctaaggcctacaag
cgcagcaagcgccagtccctgcgcgaggcccgcatcactgagaagctggagaagcagca
gaagatcgagcaggagcgcaagcgccggcagaagcaccaggaatacctcaatagcattc
tccagcatgccaaggatttcaaggaatatcacagatccgtcacaggcaaaatccagaag
ctgacca
aggcagtggccacgtaccatgccaacacggagcgggagcagaagaaagagaacgagcgg
atcgagaaggagcgcatgcggaggctcatggctgaagatgaggaggggtaccgcaagct
catcgaccagaagaaggacaagcgcctggcctacctcttgcagcagacagacgagtacg
tggctaacctcacggagctggtgcggcagcacaaggctgcccaggtcgccaaggagaaa
aagaagaaaaagaaaaagaagaaggcagaaaatgcagaaggacagacgcctgccattgg
gccggatggcgagcctctggacgagaccagccagatgagcgacctcccggtgaaggtga
tccacgtggagagtgggaagatcctcacaggcacagatgcccccaaagccgggcagctg
gaggcct
ggctcgagatgaacccggggtatgaagtagctccgaggtctgatagtgaagaaagtggc
tcagaagaagaggaagaggaggaggaggaagagcagccgcaggcagcacagcctcccac
cctgcccgtggaggagaagaagaagattccagatccagacagcgatgacgtctctgagg
tggacgcgcggcacatcattgagaatgccaagcaagatgtcgatgatgaatatggcgtg
tcccaggcccttgcacgtggcctgcagtcctactatgccgtggcccatgctgtcactga
gagagtggacaagcagtcagcgcttatggtcaatggtgtcctcaaacagtaccagatca
aaggtttggagtggctggtgtccctgtacaacaacaacctgaacggcatcctggccgac
gagatgg
gcctggggaagaccatccagaccatcgcgctcatcacgtacctcatggagcacaaacgc
atcaatgggcccttcctcatcatcgtgcctctctcaacgctgtccaactgggcgtacga
gtttgacaagtgggccccctccgtggtgaaggtgtcttacaagggatccccagcagcaa
gacgggcctttgtcccccagctccggagtgggaagttcaacgtcttgctgacgacgtac
gagtacatcatcaaagacaagcacatcctcgccaagatccgttggaagtacatgattgt
ggacgaaggtcaccgcatgaagaaccaccactgcaagctgacgcaggtgctcaacacgc
actatgtggcaccccgccgcctgctgctgacgggcacaccgctgcagaacaagcttccc
gagctct
gggcgctgctcaacttcctgctgcccaccatcttcaagagctgcagcaccttcgagcag
tggtttaacgcaccctttgccatgaccggggaaaaggtggacctgaatgaggaggaaac
cattctcatcatccggcgtctccacaaagtgctgcggcccttcttgctccgacgactca
agaaggaagtcgaggcccagttgcccgaaaaggtggagtacgtcatcaagtgcgacatg
tctgcgctgcagcgagtgctctaccgccacatgcaggccaagggcgtgctgctgactga
tggctccgagaaggacaagaagggcaaaggcggcaccaagaccctgatgaacaccatca
tgcagctgcggaagatctgcaaccacccctacatgttccagcacatcgaggagtccttt
tccgagc
acttggggttcactggcggcattgtccaagggctggacctgtaccgagcctcgggtaaa
tttgagcttcttgatagaattcttcccaaactccgagcaaccaaccacaaagtgctgct
gttctgccaaatgacctccctcatgaccatcatggaagattactttgcgtatcgcggct
ttaaatacctcaggcttgatggaaccacgaaggcggaggaccggggcatgctgctgaaa
accttcaacgagcccggctctgagtacttcatcttcctgctcagcacccgggctggggg
gctcggcctgaacctccagtcggcagacactgtgatcatttttgacagcgactggaatc
ctcaccaggacctgcaagcgcaggaccgagcccaccgcatcgggcagcagaacgaggtg
cgtgtgc
tccgcctctgcaccgtcaacagcgtggaggagaagatcctagctgcagccaagtacaag
ctcaacgtggaccagaaggtgatccaggccggcatgttcgaccagaagtcctccagcca
tgagcggcgcgccttcctgcaggccatcctggagcacgaggagcaggatgagagcagac
actgcagcacgggcagcggcagtgccagcttcgcccacactgcccctccgccagcgggc
gtcaaccccgacttggaggagccacctctaaaggaggaagacgaggtgcccgacgacga
gaccgtcaaccagatgatcgcccggcacgaggaggagtttgatctgttcatgcgcatgg
acctggaccgcaggcgcgaggaggcccgcaaccccaagcggaagccgcgcctcatggag
gaggacg
agctcccctcgtggatcatcaaggacgacgcggaggtggagcggctgacctgtgaggag
gaggaggagaagatgttcggccgtggctcccgccaccgcaaggaggtggactacagcga
ctcactgacggagaagcagtggctcaagaaaattacaggaaaagatatccatgacacag
ccagcagtgtggcacgtgggctacaattccagcgtggccttcagttctgcacacgtgcg
tcaaaggccatcgaggagggcacgctggaggagatcgaagaggaggtccggcagaagaa
atcatcacggaagcgcaagcgagacagcgacgccggctcctccaccccgaccaccagca
cccgcagccgcgacaaggacgacgagagcaagaagcagaagaagcgcgggcggccgcct
gccgaga
aactctcccctaacccacccaacctcaccaagaagatgaagaagattgtggatgccgtg
atcaagtacaaggacagcagcagtggacgtcagctcagcgaggtcttcatccagctgcc
ctcgcgaaaggagctgcccgagtactacgagctcatccgcaagcccgtggacttcaaga
agataaaggagcgcattcgcaaccacaagtaccgcagcctcaacgacctagagaaggac
gtcatgctcctgtgccagaacgcacagaccttcaacctggagggctccctgatctatga
agactccatcgtcttgcagtcggtcttcaccagcgtgcggcagaaaatcgagaaggagg
atgacagtgaaggcgaggagagtgaggaggaggaagagggcgaggaggaaggctccgaa
tccgaat
ctcggtccgtcaaagtgaagatcaagcttggccggaaggagaaggcacaggaccggctg
aagggcggccggcggcggccgagccgagggtcccgagccaagccggtcgtgagtgacga
tgacagtgaggaggaacaagaggaggaccgctcaggaagtggcagcgaagaagactgag
ccccgacattccagtctcgaccccgagcccctcgttccagagctgagatggcataggcc
ttagcagtaacgggtagcagcagatgtagtttcagacttggagtaaaactgtataaaca
aaagaatcttccatatttatacagcagagaagctgtaggactgtttgtgactggccctg
tcctggcatcagtagcatctgtaacagcattaactgtcttaaagagagagagagagaat
tccgaat
tggggaacacacgatacctgtttttcttttccgttgctggcagtactgttgcgccgcag
tttggagtcactgtagttaagtgtggatgcatgtgcgtcaccgtccactcctcctactg
tattttattggacaggtcagactcgccgggggcccggcgagggtatgtcagtgtcactg
gatgtcaaacagtaataaattaaaccaacaacaaaa
[SMARCA4 sequence-2]
SEQ ID NO: 13
gggcgcgcgcgcgaggcttcccctcgtttggcggcggcggcggcttctttgtttcgtga
agagaagcgagacgcccattctgcccccggccccgcgcggaggggcgggggaggcgccg
ggaagtcgacggcgccggcggctcctgcgtctcgcccttttgcccaggctagagtgcag
tggtgcggtcatggttcactgcagcctcaacctcctggactcagcaggaggccactgtc
tgcagctcccgtgaagatgtccactccagacccacccctgggcggaactcctcggccag
gtccttccccgggccctggcccttcccctggagccatgctgggccctagcccgggtccc
tcgccgggctccgcccacagcatgatggggcccagcccagggccgccctcagcaggaca
ccccatc
cccacccaggggcctggagggtaccctcaggacaacatgcaccagatgcacaagcccat
ggagtccatgcatgagaagggcatgtcggacgacccgcgctacaaccagatgaaaggaa
tggggatgcggtcagggggccatgctgggatggggcccccgcccagccccatggaccag
cactcccaaggttacccctcgcccctgggtggctctgagcatgcctctagtccagttcc
agccagtggcccgtcttcggggccccagatgtcttccgggccaggaggtgccccgctgg
atggtgctgacccccaggccttggggcagcagaaccggggcccaaccccatttaaccag
aaccagctgcaccagctcagagctcagatcatggcctacaagatgctggccagggggca
gcccctc
cccgaccacctgcagatggcggtgcagggcaagcggccgatgcccgggatgcagcagca
gatgccaacgctacctccaccctcggtgtccgcaacaggacccggccctggccctggcc
ctggccccggcccgggtcccggcccggcacctccaaattacagcaggcctcatggtatg
ggagggcccaacatgcctcccccaggaccctcgggcgtgccccccgggatgccaggcca
gcctcctggagggcctcccaagccctggcctgaaggacccatggcgaatgctgctgccc
ccacgagcacccctcagaagctgattcccccgcagccaacgggccgcccttcccccgcg
ccccctgccgtcccacccgccgcctcgcccgtgatgccaccgcagacccagtcccccgg
gcagccg
gcccagcccgcgcccatggtgccactgcaccagaagcagagccgcatcacccccatcca
gaagccgcggggcctcgaccctgtggagatcctgcaggagcgcgagtacaggctgcagg
ctcgcatcgcacaccgaattcaggaacttgaaaaccttcccgggtccctggccggggat
ttgcgaaccaaagcgaccattgagctcaaggccctcaggctgctgaacttccagaggca
gctgcgccaggaggtggtggtgtgcatgcggagggacacagcgctggagacagccctca
atgctaaggcctacaagcgcagcaagcgccagtccctgcgcgaggcccgcatcactgag
aagctggagaagcagcagaagatcgagcaggagcgcaagcgccggcagaagcaccagga
atacctc
aatagcattctccagcatgccaaggatttcaaggaatatcacagatccgtcacaggcaa
aatccagaagctgaccaaggcagtggccacgtaccatgccaacacggagcgggagcaga
agaaagagaacgagcggatcgagaaggagcgcatgcggaggctcatggctgaagatgag
gaggggtaccgcaagctcatcgaccagaagaaggacaagcgcctggcctacctcttgca
gcagacagacgagtacgtggctaacctcacggagctggtgcggcagcacaaggctgccc
aggtcgccaaggagaaaaagaagaaaaagaaaaagaagaaggcagaaaatgcagaagga
cagacgcctgccattgggccggatggcgagcctctggacgagaccagccagatgagcga
cctcccg
gtgaaggtgatccacgtggagagtgggaagatcctcacaggcacagatgcccccaaagc
cgggcagctggaggcctggctcgagatgaacccggggtatgaagtagctccgaggtctg
atagtgaagaaagtggctcagaagaagaggaagaggaggaggaggaagagcagccgcag
gcagcacagcctcccaccctgcccgtggaggagaagaagaagattccagatccagacag
cgatgacgtctctgaggtggacgcgcggcacatcattgagaatgccaagcaagatgtcg
atgatgaatatggcgtgtcccaggcccttgcacgtggcctgcagtcctactatgccgtg
gcccatgctgtcactgagagagtggacaagcagtcagcgcttatggtcaatggtgtcct
caaacag
taccagatcaaaggtttggagtggctggtgtccctgtacaacaacaacctgaacggcat
cctggccgacgagatgggcctggggaagaccatccagaccatcgcgctcatcacgtacc
tcatggagcacaaacgcatcaatgggcccttcctcatcatcgtgcctctctcaacgctg
tccaactgggcgtacgagtttgacaagtgggccccctccgtggtgaaggtgtcttacaa
gggatccccagcagcaagacgggcctttgtcccccagctccggagtgggaagttcaacg
tcttgctgacgacgtacgagtacatcatcaaagacaagcacatcctcgccaagatccgt
tggaagtacatgattgtggacgaaggtcaccgcatgaagaaccaccactgcaagctgac
gcaggtg
ctcaacacgcactatgtggcaccccgccgcctgctgctgacgggcacaccgctgcagaa
caagcttcccgagctctgggcgctgctcaacttcctgctgcccaccatcttcaagagct
gcagcaccttcgagcagtggtttaacgcaccctttgccatgaccggggaaaaggtggac
ctgaatgaggaggaaaccattctcatcatccggcgtctccacaaagtgctgcggccctt
cttgctccgacgactcaagaaggaagtcgaggcccagttgcccgaaaaggtggagtacg
tcatcaagtgcgacatgtctgcgctgcagcgagtgctctaccgccacatgcaggccaag
ggcgtgctgctgactgatggctccgagaaggacaagaagggcaaaggcggcaccaagac
cctgatg
aacaccatcatgcagctgcggaagatctgcaaccacccctacatgttccagcacatcga
ggagtccttttccgagcacttggggttcactggcggcattgtccaagggctggacctgt
accgagcctcgggtaaatttgagcttcttgatagaattcttcccaaactccgagcaacc
aaccacaaagtgctgctgttctgccaaatgacctccctcatgaccatcatggaagatta
ctttgcgtatcgcggctttaaatacctcaggcttgatggaaccacgaaggcggaggacc
ggggcatgctgctgaaaaccttcaacgagcccggctctgagtacttcatcttcctgctc
agcacccgggctggggggctcggcctgaacctccagtcggcagacactgtgatcatttt
tgacagc
gactggaatcctcaccaggacctgcaagcgcaggaccgagcccaccgcatcgggcagca
gaacgaggtgcgtgtgctccgcctctgcaccgtcaacagcgtggaggagaagatcctag
ctgcagccaagtacaagctcaacgtggaccagaaggtgatccaggccggcatgttcgac
cagaagtcctccagccatgagcggcgcgccttcctgcaggccatcctggagcacgagga
gcaggatgagagcagacactgcagcacgggcagcggcagtgccagcttcgcccacactg
cccctccgccagcgggcgtcaaccccgacttggaggagccacctctaaaggaggaagac
gaggtgcccgacgacgagaccgtcaaccagatgatcgcccggcacgaggaggagtttga
tctgttc
atgcgcatggacctggaccgcaggcgcgaggaggcccgcaaccccaagcggaagccgcg
cctcatggaggaggacgagctcccctcgtggatcatcaaggacgacgcggaggtggagc
ggctgacctgtgaggaggaggaggagaagatgttcggccgtggctcccgccaccgcaag
gaggtggactacagcgactcactgacggagaagcagtggctcaaggccatcgaggaggg
cacgctggaggagatcgaagaggaggtccggcagaagaaatcatcacggaagcgcaagc
gagacagcgacgccggctcctccaccccgaccaccagcacccgcagccgcgacaaggac
gacgagagcaagaagcagaagaagcgcgggcggccgcctgccgagaaactctcccctaa
cccaccc
aacctcaccaagaagatgaagaagattgtggatgccgtgatcaagtacaaggacagcag
cagtggacgtcagctcagcgaggtcttcatccagctgccctcgcgaaaggagctgcccg
agtactacgagctcatccgcaagcccgtggacttcaagaagataaaggagcgcattcgc
aaccacaagtaccgcagcctcaacgacctagagaaggacgtcatgctcctgtgccagaa
cgcacagaccttcaacctggagggctccctgatctatgaagactccatcgtcttgcagt
cggtcttcaccagcgtgcggcagaaaatcgagaaggaggatgacagtgaaggcgaggag
agtgaggaggaggaagagggcgaggaggaaggctccgaatccgaatctcggtccgtcaa
agtgaag
atcaagcttggccggaaggagaaggcacaggaccggctgaagggcggccggcggcggcc
gagccgagggtcccgagccaagccggtcgtgagtgacgatgacagtgaggaggaacaag
aggaggaccgctcaggaagtggcagcgaagaagactgagccccgacattccagtctcga
ccccgagcccctcgttccagagctgagatggcataggccttagcagtaacgggtagcag
cagatgtagtttcagacttggagtaaaactgtataaacaaaagaatcttccatatttat
acagcagagaagctgtaggactgtttgtgactggccctgtcctggcatcagtagcatct
gtaacagcattaactgtcttaaagagagagagagagaattccgaattggggaacacacg
atacctg
tttttcttttccgttgctggcagtactgttgcgccgcagtttggagtcactgtagttaa
gtgtggatgcatgtgcgtcaccgtccactcctcctactgtattttattggacaggtcag
actcgccgggggcccggcgagggtatgtcagtgtcactggatgtcaaacagtaataaat
taaaccaacaacaaaa
[ARID1A sequence-1]
SEQ ID NO: 14
ctcctttctccggcagcagaaagcggagagtcacagcggggccaggccctggggagcgg
agcctccaccgcccccctcattcccaggcaagggcttggggggaatgagccgggagagc
cgggtcccgagcctacagagccgggagcagctgagccgccggcgcctcggccgccgccg
ccgcctcctcctcctccgccgccgccagcccggagcctgagccggcggggcggggggga
gaggagcgagcgcagcgcagcagcggagccccgcgaggcccgcccgggcgggtggggag
ggcagcccgggggactgggccccggggcggggtgggagggggggagaagacgaagacag
ggccgggtctctccgcggacgagacagcggggatcatggccgcgcaggtcgcccccgcc
gccgccagcagcctgggcaacccgccgccgccgccgccctcggagctgaagaaagccga
gcagcagcagcgggaggaggcggggggcgaggcggcggcggcggcagcggccgagcgcg
gggaaatgaaggcagccgccgggcaggaaagcgagggccccgccgtggggccgccgcag
ccgctgggaaaggagctgcaggacggggccgagagcaatgggggtggcggcggcggcgg
agccggcagcggcggcgggcccggcgcggagccggacctgaagaactcgaacgggaacg
cgggccctaggcccgccctgaacaataacctcacggagccgcccggcggcggcggtggc
ggcagcagcgatggggtgggggcgcctcctcactcagccgcggccgccttgccgccccc
agcctacggcttcgggcaaccctacggccggagcccgtctgccgtcgccgccgccgcgg
ccgccgtcttccaccaacaacatggcggacaacaaagccctggcctggcagcgctgcag
agcggcggcggcgggggcctggagccctacgcggggccccagcagaactctcacgacca
cggcttccccaaccaccagtacaactcctactaccccaaccgcagcgcctaccccccgc
ccgccccggcctacgcgctgagctccccgagaggtggcactccgggctccggcgcggcg
gcggctgccggctccaagccgcctccctcctccagcgcctccgcctcctcgtcgtcttc
gtccttcgctcagcagcgcttcggggccatggggggaggcggcccctccgcggccggcg
ggggaactccccagcccaccgccacccccaccctcaaccaactgctcacgtcgcccagc
tcggcccggggctaccagggctaccccgggggcgactacagtggcgggccccaggacgg
gggcgccggcaagggcccggcggacatggcctcgcagtgttggggggctgcggcggcgg
cagctgcggcggcggccgcctcgggaggggcccaacaaaggagccaccacgcgcccatg
agccccgggagcagcggcggcggggggcagccgctcgcccggacccctcagccatccag
tccaatggatcagatgggcaagatgagacctcagccatatggcgggactaacccatact
cgcagcaacagggacctccgtcaggaccgcagcaaggacatgggtacccagggcagcca
tacgggtcccagaccccgcagcggtacccgatgaccatgcagggccgggcgcagagtgc
catgggcggcctctcttatacacagcagattcctccttatggacaacaaggccccagcg
ggtatggtcaacagggccagactccatattacaaccagcaaagtcctcaccctcagcag
cagcagccaccctactcccagcaaccaccgtcccagacccctcatgcccaaccttcgta
tcagcagcagccacagtctcaaccaccacagctccagtcctctcagcctccatactccc
agcagccatcccagcctccacatcagcagtccccggctccatacccctcccagcagtcg
acgacacagcagcacccccagagccagcccccctactcacagccacaggctcagtctcc
ttaccagcagcagcaacctcagcagccagcaccctcgacgctctcccagcaggctgcgt
atcctcagccccagtctcagcagtcccagcaaactgcctattcccagcagcgcttccct
ccaccgcaggagctatctcaagattcatttgggtctcaggcatcctcagccccctcaat
gacctccagtaagggagggcaagaagatatgaacctgagccttcagtcaagaccctcca
gcttgcctgatctatctggttcaatagatgacctccccatggggacagaaggagctctg
agtcctggagtgagcacatcagggatttccagcagccaaggagagcagagtaatccagc
tcagtctcctttctctcctcatacctcccctcacctgcctggcatccgaggcccttccc
cgtcccctgttggctctcccgccagtgttgctcagtctcgctcaggaccactctcgcct
gctgcagtgccaggcaaccagatgccacctcggccacccagtggccagtcggacagcat
catgcatccttccatgaaccaatcaagcattgcccaagatcgaggttatatgcagagga
acccccagatgccccagtacagttccccccagcccggctcagccttatctccgcgtcag
ccttccggaggacagatacacacaggcatgggctcctaccagcagaactccatggggag
ctatggtccccaggggggtcagtatggcccacaaggtggctaccccaggcagccaaact
ataatgccttgcccaatgccaactaccccagtgcaggcatggctggaggcataaacccc
atgggtgccggaggtcaaatgcatggacagcctggcatcccaccttatggcacactccc
tccagggaggatgagtcacgcctccatgggcaaccggccttatggccctaacatggcca
atatgccacctcaggttgggtcagggatgtgtcccccaccagggggcatgaaccggaaa
acccaagaaactgctgtcgccatgcatgttgctgccaactctatccaaaacaggccgcc
aggctaccccaatatgaatcaagggggcatgatgggaactggacctccttatggacaag
ggattaatagtatggctggcatgatcaaccctcagggacccccatattccatgggtgga
accatggccaacaattctgcagggatggcagccagcccagagatgatgggccttgggga
tgtaaagttaactccagccaccaaaatgaacaacaaggcagatgggacacccaaga
cagaatccaaatccaagaaatccagttcttctactacaaccaatgagaagatcaccaag
ttgtatgagctgggtggtgagcctgagaggaagatgtgggtggaccgttatctggcctt
cactgaggagaaggccatgggcatgacaaatctgcctgctgtgggtaggaaacctctgg
acctctatcgcctctatgtgtctgtgaaggagattggtggattgactcaggtcaacaag
aacaaaaaatggcgggaacttgcaaccaacctcaatgtgggcacatcaagcagtgctgc
cagctccttgaaaaagcagtatatccagtgtctctatgcctttgaatgcaagattgaac
ggggagaagaccctcccccagacatctttgcagctgctgattccaagaagtcccagccc
aagatcc
agcctccctctcctgcgggatcaggatctatgcaggggccccagactccccagtcaacc
agcagttccatggcagaaggaggagacttaaagccaccaactccagcatccacaccaca
cagtcagatccccccattgccaggcatgagcaggagcaattcagttgggatccaggatg
cctttaatgatggaagtgactccacattccagaagcggaattccatgactccaaaccct
gggtatcagcccagtatgaatacctctgacatgatggggcgcatgtcctatgagccaaa
taaggatccttatggcagcatgaggaaagctccagggagtgatcccttcatgtcctcag
ggcagggccccaacggcgggatgggtgacccctacagtcgtgctgccggccctgggcta
ggaaatg
tggcgatgggaccacgacagcactatccctatggaggtccttatgacagagtgaggacg
gagcctggaatagggcctgagggaaacatgagcactggggccccacagccgaatctcat
gccttccaacccagactcggggatgtattctcctagccgctaccccccgcagcagcagc
agcagcagcagcaacgacatgattcctatggcaatcagttctccacccaaggcacccct
tctggcagccccttccccagccagcagactacaatgtatcaacagcaacagcagaatta
caagcggccaatggatggcacatatggccctcctgccaagcggcacgaaggggagatgt
acagcgtgccatacagcactgggcaggggcagcctcagcagcagcagttgcccccagcc
cagccccagcctgccagccagcaacaagctgcccagccttcccctcagcaagatgtata
caaccagtatggcaatgcctatcctgccactgccacagctgctactgagcgccgaccag
caggcggcccccagaaccaatttccattccagtttggccgagaccgtgtctctgcaccc
cctggcaccaatgcccagcaaaacatgccaccacaaatgatgggcggccccatacaggc
atcagctgaggttgctcagcaaggcaccatgtggcaggggcgtaatgacatgacctata
attatgccaacaggcagagcacgggctctgccccccagggccccgcctatcatggcgtg
aaccgaacagatgaaatgctgcacacagatcagagggccaaccacgaaggctcgtggcc
ttcccatggcacacgccagcccccatatggtccctctgcccctgtgccccccatgacaa
ggccccctccatctaactaccagcccccaccaagcatgcagaatcacattcctcaggta
tccagccctgctcccctgccccggccaatggagaaccgcacctctcctagcaagtctcc
attcctgcactctgggatgaaaatgcagaaggcaggtcccccagtacctgcctcgcaca
tagcacctgcccctgtgcagccccccatgattcggcgggatatcaccttcccacctggc
tctgttgaagccacacagcctgtgttgaagcagaggaggcggctcacaatgaaagacat
tggaaccccggaggcatggcgggtaatgatgtccctcaagtctggtctcctggcagaga
gcacatgggcattagatacca
tcaacatcctgctgtatgatgacaacagcatcatgaccttcaacctcagtcagctccca
gggttgctagagctccttgtagaatatttccgacgatgcctgattgagatctttggcat
tttaaaggagtatgaggtgggtgacccaggacagagaacgctactggatcctgggaggt
tcagcaaggtgtctagtccagctcccatggagggtggggaagaagaagaagaacttcta
ggtcctaaactagaagaggaagaagaagaggaagtagttgaaaatgatgaggagatagc
cttttcaggcaaggacaagccagcttcagagaatagtgaggagaagctgatcagtaagt
ttgacaagcttccagtaaagatcgtacagaagaatgatccatttgtggtggactgctca
gataagc
ttgggcgtgtgcaggagtttgacagtggcctgctgcactggcggattggtgggggggac
accactgagcatatccagacccacttcgagagcaagacagagctgctgccttcccggcc
tcacgcaccctgcccaccagcccctcggaagcatgtgacaacagcagagggtacaccag
ggacaacagaccaggaggggcccccacctgatggacctccagaaaaacggatcacagcc
actatggatgacatgttgtctactcggtctagcaccttgaccgaggatggagctaagag
ttcagaggccatcaaggagagcagcaagtttccatttggcattagcccagcacagagcc
accggaacatcaagatcctagaggacgaaccccacagtaaggatgagaccccactgtgt
acccttctggactggcaggattctcttgccaagcgctgcgtctgtgtgtccaataccat
tcgaagcctgtcatttgtgccaggcaatgactttgagatgtccaaacacccagggctgc
tgctcatcctgggcaagctgatcctgctgcaccacaagcacccagaacggaagcaggca
ccactaacttatgaaaaggaggaggaacaggaccaaggggtgagctgcaacaaagtgga
gtggtggtgggactgcttggagatgctccgggaaaacaccttggttacactcgccaaca
tctcggggcagttggacctatctccataccccgagagcatttgcctgcctgtcctggac
ggactcctacactgggcagtttgcccttcagctgaagcccaggaccccttttccaccct
gggccccaatgccgtcctttccccgcagagactggtcttggaaaccctcagcaaactca
gcatccaggacaacaatgtggacctgattctggccacaccccccttcagccgcctggag
aagttgtatagcactatggtgcgcttcctcagtgaccgaaagaacccggtgtgccggga
gatggctgtggtactgctggccaacctggctcagggggacagcctggcagctcgtgcca
ttgcagtgcagaagggcagtatcggcaacctcctgggcttcctagaggacagccttgcc
gccacacagttccagcagagccaggccagcctcctccacatgcagaacccaccctttga
gccaactagtgtggacatgatgcggcgggctgcccgcgcgctgcttgccttggccaagg
tggacgagaaccactcagagtttactctgtacgaatcacggctgttggacatctcggta
tcaccgttgatgaactcattggtttcacaagtcatttgtgatgtactgtttttgattgg
ccagtcatgacagccgtgggacacctcccccccccgtgtgtgtgtgcgtgtgtggagaa
cttagaaactgactgttgccctttatttatgcaaaaccacctcagaatccagtttaccc
tgtgctgtccagcttctcccttgggaaaaagtctctcctgtttctctctcctccttcca
cctcccctccctccatcacctcacgcctttctgttccttgtcctcaccttactcccctc
aggaccctaccccaccctctttgaaaagacaaagctctgcctacatagaagactttttt
tattttaaccaaagttactgttgtttac
agtgagtttggggaaaaaaaataaaataaaaatggctttcccagtccttgcatcaacgg
gatgccacatttcataactgtttttaatggtaaaaaaaaaaaaaaaaaatacaaaaaaa
aattctgaaggacaaaaaaggtgactgctgaactgtgtgtggtttattgttgtacattc
acaatcttgcaggagccaagaagttcgcagttgtgaacagaccctgttcactggagagg
cctgtgcagtagagtgtagaccctttcatgtactgtactgtacacctgatactgtaaac
atactgtaataataatgtctcacatggaaacagaaaacgctgggtcagcagcaagctgt
agtttttaaaaatgtttttagttaaacgttgaggagaaaaaaaaaaaaggcttttcccc
caaagtatcatgtgtgaacctacaacaccctgacctctttctctcctccttgattgtat
gaataaccctgagatcacctcttagaactggttttaacctttagctgcagcggctacgc
tgccacgtgtgtatatatatgacgttgtacattgcacatacccttggatccccacagtt
tggtcctcctcccagctacccctttatagtatgacgagttaacaagttggtgacctgca
caaagcgagacacagctatttaatctcttgccagatatcgcccctcttggtgcgatgct
gtacaggtctctgtaaaaagtccttgctgtctcagcagccaatcaacttatagtttatt
tttttctgggtttttgttttgttttgttttctttctaatcgaggtgtgaaaaagttcta
ggttcagttgaagt
tctgatgaagaaacacaattgagattttttcagtgataaaatctgcatatttgtatttc
aacaatgtagctaaaacttgatgtaaattcctcctttttttccttttttggcttaatga
atatcatttattcagtatgaaatctttatactatatgttccacgtgttaagaataaatg
tacattaaatcttggtaa
[ARID1A sequence-2]
SEQ ID NO: 15
ctcctttctccggcagcagaaagcggagagtcacagcggggccaggccctggggagcgg
agcctccaccgcccccctcattcccaggcaagggcttggggggaatgagccgggagagc
cgggtcccgagcctacagagccgggagcagctgagccgccggcgcctcggccgccgccg
ccgcctcctcctcctccgccgccgccagcccggagcctgagccggcggggcggggggga
gaggagcgagcgcagcgcagcagcggagccccgcgaggcccgcccgggcgggtggggag
ggcagcccgggggactgggccccggggcggggtgggagggggggagaagacgaagacag
ggccgggtctctccgcggacgagacagcggggatcatggccgcgcaggtcgcccccgcc
gccgccagcagcctgggcaacccgccgccgccgccgccctcggagctgaagaaagccga
gcagcagcagcgggaggaggcggggggcgaggcggcggcggcggcagcggccgagcgcg
gggaaatgaaggcagccgccgggcaggaaagcgagggccccgccgtggggccgccgcag
ccgctgggaaaggagctgcaggacggggccgagagcaatgggggtggcggcggcggcgg
agccggcagcggcggcgggcccggcgcggagccggacctgaagaactcgaacgggaacg
cgggccctaggcccgccctgaacaataacctcacggagccgcccggcggcggcggtggc
ggcagcagcgatggggtgggggcgcctcctcactcagccgcggccgccttgccgccccc
agcctacggcttcg
ggcaaccctacggccggagcccgtctgccgtcgccgccgccgcggccgccgtcttccac
caacaacatggcggacaacaaagccctggcctggcagcgctgcagagcggcggcggcgg
gggcctggagccctacgcggggccccagcagaactctcacgaccacggcttccccaacc
accagtacaactcctactaccccaaccgcagcgcctaccccccgcccgccccggcctac
gcgctgagctccccgagaggtggcactccgggctccggcgcggcggcggctgccggctc
caagccgcctccctcctccagcgcctccgcctcctcgtcgtcttcgtccttcgctcagc
agcgcttcggggccatggggggaggcggcccctccgcggccggcgggggaactccccag
cccaccg
ccacccccaccctcaaccaactgctcacgtcgcccagctcggcccggggctaccagggc
taccccgggggcgactacagtggcgggccccaggacgggggcgccggcaagggcccggc
ggacatggcctcgcagtgttggggggctgcggcggcggcagctgcggcggcggccgcct
cgggaggggcccaacaaaggagccaccacgcgcccatgagccccgggagcagcggcggc
ggggggcagccgctcgcccggacccctcagccatccagtccaatggatcagatgggcaa
gatgagacctcagccatatggcgggactaacccatactcgcagcaacagggacctccgt
caggaccgcagcaaggacatgggtacccagggcagccatacgggtcccagaccccgcag
cggtacc
cgatgaccatgcagggccgggcgcagagtgccatgggcggcctctcttatacacagcag
attcctccttatggacaacaaggccccagcgggtatggtcaacagggccagactccata
ttacaaccagcaaagtcctcaccctcagcagcagcagccaccctactcccagcaaccac
cgtcccagacccctcatgcccaaccttcgtatcagcagcagccacagtctcaaccacca
cagctccagtcctctcagcctccatactcccagcagccatcccagcctccacatcagca
gtccccggctccatacccctcccagcagtcgacgacacagcagcacccccagagccagc
ccccctactcacagccacaggctcagtctccttaccagcagcagcaacctcagcagcca
gcaccct
cgacgctctcccagcaggctgcgtatcctcagccccagtctcagcagtcccagcaaact
gcctattcccagcagcgcttccctccaccgcaggagctatctcaagattcatttgggtc
tcaggcatcctcagccccctcaatgacctccagtaagggagggcaagaagatatgaacc
tgagccttcagtcaagaccctccagcttgcctgatctatctggttcaatagatgacctc
cccatggggacagaaggagctctgagtcctggagtgagcacatcagggatttccagcag
ccaaggagagcagagtaatccagctcagtctcctttctctcctcatacctcccctcacc
tgcctggcatccgaggcccttccccgtcccctgttggctctcccgccagtgttgctcag
tctcgctcaggaccactctcgcctgctgcagtgccaggcaaccagatgccacctcggcc
acccagtggccagtcggacagcatcatgcatccttccatgaaccaatcaagcattgccc
aagatcgaggttatatgcagaggaacccccagatgccccagtacagttccccccagccc
ggctcagccttatctccgcgtcagccttccggaggacagatacacacaggcatgggctc
ctaccagcagaactccatggggagctatggtccccaggggggtcagtatggcccacaag
gtggctaccccaggcagccaaactataatgccttgcccaatgccaactaccccagtgca
ggcatggctggaggcataaaccccatgggtgccggaggtcaaatgcatggacagcctgg
catcccaccttatg
gcacactccctccagggaggatgagtcacgcctccatgggcaaccggccttatggccct
aacatggccaatatgccacctcaggttgggtcagggatgtgtcccccaccagggggcat
gaaccggaaaacccaagaaactgctgtcgccatgcatgttgctgccaactctatccaaa
acaggccgccaggctaccccaatatgaatcaagggggcatgatgggaactggacctcct
tatggacaagggattaatagtatggctggcatgatcaaccctcagggacccccatattc
catgggtggaaccatggccaacaattctgcagggatggcagccagcccagagatgatgg
gccttggggatgtaaagttaactccagccaccaaaatgaacaacaaggcagatgggaca
cccaaga
cagaatccaaatccaagaaatccagttcttctactacaaccaatgagaagatcaccaag
ttgtatgagctgggtggtgagcctgagaggaagatgtgggtggaccgttatctggcctt
cactgaggagaaggccatgggcatgacaaatctgcctgctgtgggtaggaaacctctgg
acctctatcgcctctatgtgtctgtgaaggagattggtggattgactcaggtcaacaag
aacaaaaaatggcgggaacttgcaaccaacctcaatgtgggcacatcaagcagtgctgc
cagctccttgaaaaagcagtatatccagtgtctctatgcctttgaatgcaagattgaac
ggggagaagaccctcccccagacatctttgcagctgctgattccaagaagtcccagccc
aagatccagcctccctctcctgcgggatcaggatctatgcaggggccccagactcccca
gtcaaccagcagttccatggcagaaggaggagacttaaagccaccaactccagcatcca
caccacacagtcagatccccccattgccaggcatgagcaggagcaattcagttgggatc
caggatgcctttaatgatggaagtgactccacattccagaagcggaattccatgactcc
aaaccctgggtatcagcccagtatgaatacctctgacatgatggggcgcatgtcctatg
agccaaataaggatccttatggcagcatgaggaaagctccagggagtgatcccttcatg
tcctcagggcagggccccaacggcgggatgggtgacccctacagtcgtgctgccggccc
tgggctaggaaatg
tggcgatgggaccacgacagcactatccctatggaggtccttatgacagagtgaggacg
gagcctggaatagggcctgagggaaacatgagcactggggccccacagccgaatctcat
gccttccaacccagactcggggatgtattctcctagccgctaccccccgcagcagcagc
agcagcagcagcaacgacatgattcctatggcaatcagttctccacccaaggcacccct
tctggcagccccttccccagccagcagactacaatgtatcaacagcaacagcaggtatc
cagccctgctcccctgccccggccaatggagaaccgcacctctcctagcaagtctccat
tcctgcactctgggatgaaaatgcagaaggcaggtcccccagtacctgcctcgcacata
gcacctgcccctgtgcagccccccatgattcggcgggatatcaccttcccacctggctc
tgttgaagccacacagcctgtgttgaagcagaggaggcggctcacaatgaaagacattg
gaaccccggaggcatggcgggtaatgatgtccctcaagtctggtctcctggcagagagc
acatgggcattagataccatcaacatcctgctgtatgatgacaacagcatcatgacctt
caacctcagtcagctcccagggttgctagagctccttgtagaatatttccgacgatgcc
tgattgagatctttggcattttaaaggagtatgaggtgggtgacccaggacagagaacg
ctactggatcctgggaggttcagcaaggtgtctagtccagctcccatggagggtgggga
agaagaagaagaac
ttctaggtcctaaactagaagaggaagaagaagaggaagtagttgaaaatgatgaggag
atagccttttcaggcaaggacaagccagcttcagagaatagtgaggagaagctgatcag
taagtttgacaagcttccagtaaagatcgtacagaagaatgatccatttgtggtggact
gctcagataagcttgggcgtgtgcaggagtttgacagtggcctgctgcactggcggatt
ggtgggggggacaccactgagcatatccagacccacttcgagagcaagacagagctgct
gccttcccggcctcacgcaccctgcccaccagcccctcggaagcatgtgacaacagcag
agggtacaccagggacaacagaccaggaggggcccccacctgatggacctccagaaaaa
cggatcacagccactatggatgacatgttgtctactcggtctagcaccttgaccgagga
tggagctaagagttcagaggccatcaaggagagcagcaagtttccatttggcattagcc
cagcacagagccaccggaacatcaagatcctagaggacgaaccccacagtaaggatgag
accccactgtgtacccttctggactggcaggattctcttgccaagcgctgcgtctgtgt
gtccaataccattcgaagcctgtcatttgtgccaggcaatgactttgagatgtccaaac
acccagggctgctgctcatcctgggcaagctgatcctgctgcaccacaagcacccagaa
cggaagcaggcaccactaacttatgaaaaggaggaggaacaggaccaaggggtgagctg
caacaaagtggagt
ggtggtgggactgcttggagatgctccgggaaaacaccttggttacactcgccaacatc
tcggggcagttggacctatctccataccccgagagcatttgcctgcctgtcctggacgg
actcctacactgggcagtttgcccttcagctgaagcccaggaccccttttccaccctgg
gccccaatgccgtcctttccccgcagagactggtcttggaaaccctcagcaaactcagc
atccaggacaacaatgtggacctgattctggccacaccccccttcagccgcctggagaa
gttgtatagcactatggtgcgcttcctcagtgaccgaaagaacccggtgtgccgggaga
tggctgtggtactgctggccaacctggctcagggggacagcctggcagctcgtgccatt
gcagtgc
agaagggcagtatcggcaacctcctgggcttcctagaggacagccttgccgccacacag
ttccagcagagccaggccagcctcctccacatgcagaacccaccctttgagccaactag
tgtggacatgatgcggcgggctgcccgcgcgctgcttgccttggccaaggtggacgaga
accactcagagtttactctgtacgaatcacggctgttggacatctcggtatcaccgttg
atgaactcattggtttcacaagtcatttgtgatgtactgtttttgattggccagtcatg
acagccgtgggacacctcccccccccgtgtgtgtgtgcgtgtgtggagaacttagaaac
tgactgttgccctttatttatgcaaaaccacctcagaatccagtttaccctgtgctgtc
cagcttc
tcccttgggaaaaagtctctcctgtttctctctcctccttccacctcccctccctccat
cacctcacgcctttctgttccttgtcctcaccttactcccctcaggaccctaccccacc
ctctttgaaaagacaaagctctgcctacatagaagactttttttattttaaccaaagtt
actgttgtttacagtgagtttggggaaaaaaaataaaataaaaatggctttcccagtcc
ttgcatcaacgggatgccacatttcataactgtttttaatggtaaaaaaaaaaaaaaaa
aatacaaaaaaaaattctgaaggacaaaaaaggtgactgctgaactgtgtgtggtttat
tgttgtacattcacaatcttgcaggagccaagaagttcgcagttgtgaacagaccctgt
tcactgg
agaggcctgtgcagtagagtgtagaccctttcatgtactgtactgtacacctgatactg
taaacatactgtaataataatgtctcacatggaaacagaaaacgctgggtcagcagcaa
gctgtagtttttaaaaatgtttttagttaaacgttgaggagaaaaaaaaaaaaggcttt
tcccccaaagtatcatgtgtgaacctacaacaccctgacctctttctctcctccttgat
tgtatgaataaccctgagatcacctcttagaactggttttaacctttagctgcagcggc
tacgctgccacgtgtgtatatatatgacgttgtacattgcacatacccttggatcccca
cagtttggtcctcctcccagctacccctttatagtatgacgagttaacaagttggtgac
ctgcaca
aagcgagacacagctatttaatctcttgccagatatcgcccctcttggtgcgatgctgt
acaggtctctgtaaaaagtccttgctgtctcagcagccaatcaacttatagtttatttt
tttctgggtttttgttttgttttgttttctttctaatcgaggtgtgaaaaagttctagg
ttcagttgaagttctgatgaagaaacacaattgagattttttcagtgataaaatctgca
tatttgtatttcaacaatgtagctaaaacttgatgtaaattcctcctttttttcctttt
ttggcttaatgaatatcatttattcagtatgaaatctttatactatatgttccacgtgt
taagaataaatgtacattaaatcttggtaa
[ARID1B sequence-1]
SEQ ID NO: 16
acacatgcatagtgagattgctctacaaagggcagcttcccctcactgtctgtcagtaa
aagaccaaaaggcatatgcaacatgttgtgatttaggttgaagtcttggcctcggagga
tgcagcctttggactcaaggatctgtctggctccattgatgacctccccacgggaacgg
aagcaactttgagctcagcagtcagtgcatccgggtccacgagcagccaaggggatcag
agcaacccggcgcagtcgcctttctccccacatgcgtcccctcatctctccagcatccc
ggggggcccatctccctctcctgttggctctcctgtaggaagcaaccagtctcgatctg
gcccaatctctcctgcaagtatcccaggtagtcagatgcctccgcagccacccgggagc
cagtcag
aatccagttcccatcccgccttgagccagtcaccaatgccacaggaaagaggttttatg
gcaggcacacaaagaaaccctcagatggctcagtatggacctcaacagacaggaccatc
catgtcgcctcatccttctcctgggggccagatgcatgctggaatcagtagctttcagc
agagtaactcaagtgggacttacggtccacagatgagccagtatggaccacaaggtaac
tactccagacccccagcgtatagtggggtgcccagtgcaagctacagcggcccagggcc
cggtatgggtatcagtgccaacaaccagatgcatggacaagggccaagccagccatgtg
gtgctgtgcccctgggacgaatgccatcagctgggatgcagaacagaccatttcctgga
aatatga
gcagcatgacccccagttctcctggcatgtctcagcagggagggccaggaatggggccg
ccaatgccaactgtgaaccgtaaggcacaggaggcagccgcagcagtgatgcaggctgc
tgcgaactcagcacaaagcaggcaaggcagtttccccggcatgaaccagagtggactta
tggcttccagctctccctacagccagcccatgaacaacagctctagcctgatgaacacg
caggcgccgccctacagcatggcgcccgccatggtgaacagctcggcagcatctgtggg
tcttgcagatatgatgtctcctggtgaatccaaactgcccctgcctctcaaagcagacg
gcaaagaagaaggcactccacagcccgagagcaagtcaaaggatagctacagctctcag
ggtattt
ctcagcccccaaccccaggcaacctgccagtcccttccccaatgtcccccagctctgct
agcatctcctcatttcatggagatgaaagtgatagcattagcagcccaggctggccaaa
gactccatcaagccctaagtccagctcctccaccactactggggagaagatcacgaagg
tgtacgagctggggaatgagccagagagaaagctctgggtcgaccgatacctcaccttc
atggaagagagaggctctcctgtctcaagtctgcctgccgtgggcaagaagcccctgga
cctgttccgactctacgtctgcgtcaaagagatcgggggtttggcccaggttaataaaa
acaagaagtggcgtgagctggcaaccaacctaaacgttggcacctcaagcagtgcagcg
agctccc
tgaaaaagcagtatattcagtacctgtttgcctttgagtgcaagatcgaacgtggggag
gagcccccgccggaagtcttcagcaccggggacaccaaaaagcagcccaagctccagcc
gccatctcctgctaactcgggatccttgcaaggcccacagaccccccagtcaactggca
gcaattccatggcagaggttccaggtgacctgaagccacctaccccagcctccacccct
cacggccagatgactccaatgcaaggtggaagaagcagtacaatcagtgtgcacgaccc
attctcagatgtgagtgattcatccttcccgaaacggaactccatgactccaaacgccc
cctaccagcagggcatgagcatgcccgatgtgatgggcaggatgccctatgagcccaac
aaggacc
cctttgggggaatgagaaaagtgcctggaagcagcgagccctttatgacgcaaggacag
atgcccaacagcagcatgcaggacatgtacaaccaaagtccctccggagcaatgtctaa
cctgggcatggggcagcgccagcagtttccctatggagccagttacgaccgaaggcatg
aaccttatgggcagcagtatccaggccaaggccctccctcgggacagccgccgtatgga
gggcaccagcccggcctgtacccacagcagccgaattacaaacgccatatggacggcat
gtacgggcccccagccaagcgccacgagggcgacatgtacaacatgcagtacagcagcc
agcagcaggagatgtacaaccagtatggaggctcctactcgggcccggaccgcaggccc
atccagg
gccagtacccgtatccctacagcagggagaggatgcagggcccggggcagatccagaca
cacggaatcccgcctcagatgatgggcggcccgctgcagtcgtcctccagtgaggggcc
tcagcagaatatgtgggcagcacgcaatgatatgccttatccctaccagaacaggcagg
gccctggcggccctacacaggcgcccccttacccaggcatgaaccgcacagacgatatg
atggtacccgatcagaggataaatcatgagagccagtggccttctcacgtcagccagcg
tcagccttatatgtcgtcctcagcctccatgcagcccatcacacgcccaccacagccgt
cctaccagacgccaccgtcactgccaaatcacatctccagggcgcccagcccagcgtcc
ttccagc
gctccctggagaaccgcatgtctccaagcaagtctccttttctgccgtctatgaagatg
cagaaggtcatgcccacggtccccacatcccaggtcaccgggccaccaccccaaccacc
cccaatcagaagggagatcacctttcctcctggctcagtagaagcatcacaaccagtct
tgaaacaaaggcgaaagattacctccaaagatatcgttactcctgaggcgtggcgtgtg
atgatgtcccttaaatcaggtcttttggctgagagtacgtgggctttggacactattaa
tattcttctgtatgatgacagcactgttgctactttcaatctctcccagttgtctggat
ttctcgaacttttagtcgagtactttagaaaatgcctgattgacatttttggaattctt
atggaat
atgaagtgggagaccccagccaaaaagcacttgatcacaacgcagcaaggaaggatgac
agccagtccttggcagacgattctgggaaagaggaggaagatgctgaatgtattgatga
cgacgaggaagacgaggaggatgaggaggaagacagcgagaagacagaaagcgatgaaa
agagcagcatcgctctgactgccccggacgccgctgcagacccaaaggagaagcccaag
caagccagtaagttcgacaagctgccaataaagatagtcaaaaagaacaacctgtttgt
tgttgaccgatctgacaagttggggcgtgtgcaggagttcaatagtggccttctgcact
ggcagctcggcgggggtgacaccaccgagcacattcagactcactttgagagcaagatg
gaaattc
ctcctcgcaggcgcccacctccccccttaagctccgcaggtagaaagaaagagcaagaa
ggcaaaggcgactctgaagagcagcaagagaaaagcatcatagcaaccatcgatgacgt
cctctctgctcggccaggggcattgcctgaagacgcaaaccctgggccccagaccgaaa
gcagtaagtttccctttggtatccagcaagccaaaagtcaccggaacatcaagctgctg
gaggacgagcccaggagccgagacgagactcctctgtgtaccatcgcgcactggcagga
ctcgctggctaagcgatgcatctgtgtgtccaatattgtccgtagcttgtcattcgtgc
ctggcaatgatgccgaaatgtccaaacatccaggcctggtgctgatcctggggaagctg
attcttcttcaccacgagcatccagagagaaagcgagcaccgcagacctatgagaaaga
ggaggatgaggacaagggggtggcctgcagcaaagatgagtggtggtgggactgcctcg
aggtcttgagggataacacgttggtcacgttggccaacatttccgggcagctagacttg
tctgcttacacggaaagcatctgcttgccaattttggatggcttgctgcactggatggt
gtgcccgtctgcagaggcacaagatccctttccaactgtgggacccaactcggtcctgt
cgcctcagagacttgtgctggagaccctctgtaaactcagtatccaggacaataatgtg
gacctgatcttggccactcctccatttagtcgtcaggagaaattctatgctacattagt
taggtacgttggggatcgcaaaaacccagtctgtcgagaaatgtccatggcgcttttat
cgaaccttgcccaaggggacgcactagcagcaagggccatagctgtgcagaaaggaagc
attggaaacttgataagcttcctagaggatggggtcacgatggcccagtaccagcagag
ccagcacaacctcatgcacatgcagcccccgcccctggaaccacctagcgtagacatga
tgtgcagggcggccaaggctttgctagccatggccagagtggacgaaaaccgctcggaa
ttccttttgcacgagggccggttgctggatatctcgatatcagctgtcctgaactctct
ggttgcatctgtcatctgtgatgtactgtttcagattgggcagttatgacataagtgag
aaggcaagcatgtgtgagtgaagattagagggtcacatataactggctgttttctgttc
ttgtttatccagcgtaggaagaaggaaaagaaaatctttgctcctctgccccattcact
atttaccaattgggaattaaagaaataattaatttgaacagttatgaaattaatatttg
ctgtctgtgtgtataagtacatcctttggggttttttttttctcttttttttaaccaaa
gttgctgtctagtgcattcaaaggtcactttttgttcttcacagatctttttaatgttc
tttcccatgttgtattgcatttttgggggaagcaaattgactttaaagaaaaaagttgt
ggcaaaagatgctaagatgcgaaaatttcaccacactgagtcaaaaaggtgaaaaatta
tccatttcctatgcgttttactcctcag
agaatgaaaaaaactgcatcccatcacccaaagttctgtgcaatagaaatttctacaga
tacaggtataggggctcaaggaggtatgtcggtcagtagtcaaaactatgaaatgatac
tggtttctccacaggaatatggttccattaggctgggagcaaaaacaatgttttttaag
attgagaatacatacctgacaacgatccggaaactgctcctcaccactcccgtcatgcc
tgctgtcggcgtttgaccttccacgtgacagttcttcacaattcctttcatcatttttt
aaatattttttttactgcctatgggctgtgatgtatatagaagttgtacattaaacata
ccctcatttttttcttttcttttttttttttttttttagtacaaagttttagtttcttt
ttcatga
tgtggtaactacgaagtgatggtagatttaaataattttttatttttattttatatatt
ttttcattagggccatatctccaaaaaaagaaagaaaaaatacaaaaaacaaaaacaaa
aaaaaaagagggtaatgtacaagtttctgtatgtataaagtcatgctcgatttcaggag
agcagctgatcacaatttgcttcatgaatcaaggtgtggaaatggttatatatggattg
atttagaaaatggttaccagtacagtcaaaaaagagaaaatgaaaaaaatacaactaaa
aggaagaaacacaacttcaaagatttttcagtgatgagaatccacatttgtatttcaag
ataatgtagtttaaaaaaaaaaaaaagaaaaaaacttgatgtaaattcctccttttcct
ctggctt
aatgaatatcatttattcagtataaaatctttatatgttccacatgttaagaataaatg
tacattaaatcttgttaagcactgtgatgggtgttcttgaatactgttctagtttcctt
aaagtggtttcctagtaatcaagttatttacaagaaataggggaatgcagcagtgtatt
cacattataaaaccctacatttggaagagacctttaggggttacctactttagagtggg
gagcaacagtttgattttctcaaattacttagctaattagtctttctttgaagcaatta
actctaacgacattgaggtatgatcattttcagtatttatgggaggtggctgctgaccc
acttgaggtgagatctcagaagcttaactggcctgaaaatgtaacattctgccttttac
taactcc
atcttagtttaatcaaagttcaatctattccttgtttcttctgtgtgcctcagagttat
tttgcatttagtttactccaccgtgtataatatttatactgtgcaatgttaaaaaagaa
tctgttatattgtatgtggtgtacatagtgcaaagtgatgatttctatttcagggcata
ttatggttctcatattccttcctacctggtgcacagtagctttttaatactagtcactt
ctaatttaaactttctcttcctgggtcattgactgttactgtgtaataatcgatttctt
tgaaactgctgcataattatgctgttagtggacctctacctcttctcttccctctccca
atcacagtatactcagaatccccagcccctcgcatacattgtgtcggttcacattactc
acagtaa
tatatggaagagttagacaagaacatgcagttacagtcattgtgagacgtgactctcca
gtgtcacgaggaaaaaaatcatcttttctgcaaacagtctctcatctgtcaactcccac
attactgagtcaaacagtcttcttacataacaatgcaaccaaatatatgttgaattaaa
gacccatttataattctgctttaaatacatctgcttgctaagaacagatttcagtgctc
caagcttcaaatatggagatttgtaagagggaattcaatattattctaatttctctctt
acagagtacaaataaaaggtgtatacaaactccgaacatatccagtattccaattcctt
tgtcaatcagaagagtaaaataattaacaaaagactgttgttatggtttgcattgtaac
cgatacg
cagagtctgaccgttgggcaacaagtttttctatcctgatgcgcaacacagtctctaga
gactaatccaggaagactttagcctcctttccatattctcacccccgaatcaagattta
cagaagcccacgaagaatttacagcctgcttgagatcatcttgcctataaactgagtta
ttgctttgtcctaaaaattagtcggtttttttttttctatgaggcttttcagaaattta
caggatgcccagactttacatgtgtaccaaaaaaaaaaaaaagataaaaaataaaggtg
caaagaaagtttagtattttggaatggtgctataaagttgaa
[ARID1B sequence-2]
SEQ ID NO: 17
gtcaaacaaataaccactgctgcagtggaaatgaactaaaatagtttttatggaaagct
gaagaatgaaataattttggaaaactatactctaccaaaaccttggaagaagttgtggg
aattacgccgatggcccgaaatcaaggcagaggcgaacgttctgaaaattacataactc
ctgggagagaccgtgcttttgcagattgcctaaatattttctttctccgtcggctcctt
gtaacctctgaggccaatagacaactttgtttcccagcccttctccctcttttgtctgc
tcgtcagactcgttttccacctttgtatcgttccttttagacgtgaaaacaaacatgcg
aacgcccggttggcaaggagacagcggcccggggggaaagtgcaggttccggccgcccc
acgccgc
cgcgctccgagcggccgccgagccgccccggccccggccccggccccagccttagccca
agcccggctgggtcccgcgaggctgcgccggggcgggcggcggcggcccggggacgagt
ccagtccgcgttttgcgagtgcgcgggagtaatgcgagcgaagtggataatagttgctc
gagctcgcccgctgcctctcaagccatgctgggcccgataggctcagctagtcgtgtat
ttacccatatccgggctagagaggaaaagagaaaagtttcatttaaacctgaactaaaa
actttcaccatgaaagcacacagcaggagcaggcccagagcgtaaggcgtgcccggccc
ggcgctccggcggggcctgcggagggggagggggtcgcggcttcccggcgggccgcgtg
gatgcgcacaggaggggccgcggcctgaaaagtgggggttattgtctccccccgccccc
cgcccggcctcgccacgccgcggcgatcatggccgcgcgggcagcagcggcggcggcgg
cggcggcggcgcgggcgcgggcgcgggcaggcagcggcgaacggcgggcgccccccggg
ccgcggccggcgcccggagcccgggacctggaggcgggggcgcgcggcgcggcggcggc
ggcggcggcaccgggacccatgctggggggcggcggcgacggcggcggcggcctgaaca
gtgtgcaccaccaccccctgctcccccgtcacgaactcaacatggcccataacgcgggc
gccgcggccgccgccggcacccacagcgccaagagcggcggctccgaggcggctctcaa
ggagggtggaagcgccgccgcgctgtcctcctcctcctcctcctccgcggcggcagcgg
cggcatcctcttcctcctcgtcgggcccgggctcggccatggagacggggctgctcccc
aaccacaaactgaaaaccgttggcgaagcccccgccgcgccgccccaccagcagcacca
ccaccaccaccatgcccaccaccaccaccaccatgcccaccacctccaccaccaccacg
cactacagcagcagctaaaccagttccagcagcagcagcagcagcagcaacagcagcag
cagcagcagcagcaacagcaacatcccatttccaacaacaacagcttgggcggcgcggg
cggcggcgcgcctcagcccggccccgacatggagcagccgcaacatggaggcgccaagg
acagtgctgcgggcggccaggccgaccccccgggcccgccgctgctgagcaagccgggc
gacgaggacgacgcgccgcccaagatgggggagccggcgggcggccgctacgagcaccc
gggcttgggcgccctgggcacgcagcagccgccggtcgccgtgcccgggggcggcggcg
gcccggcggccgtcccggagtttaataattactatggcagcgctgcccctgcgagcggc
ggccccggcggccgcgctgggccttgctttgatcaacatggcggacaacaaagccccgg
gatggggatgatgcactccgcctccgccgccgccgccggggcccccggcagcatggacc
ccctgcagaactcccacgaagggtaccccaacagccagtgcaaccattatccgggctac
agccggcccggcgcgggcggcggcggcggcggcggcggcggaggaggaggaggcagcgg
aggaggaggaggaggaggaggagcaggagcaggaggagcaggagcgggagctgtggcgg
cggcggccgcggcggcggcggcagcagcaggaggcggcggcggcggcggctatgggggc
tcgtccgcggggtacggggtgctgagctccccccggcagcagggcggcggcatgatgat
gggccccgggggcggcggggccgcgagcctcagcaaggcggccgccggctcggcggcgg
ggggcttccagcgcttcgccggccagaaccagcacccgtcgggggccaccccgaccctc
aatcagctgctcacctcgcccagccccatgatgcggagctacggcggcagctaccccga
gtacagcagccccagcgcgccgccgccgccgccgtcgcagccccagtcccaggcggcgg
cggcgggggcggcggcgggcggccagcaggcggccgcgggcatgggcttgggcaaggac
atgggcgcccagtacgccgctgccagcccggcctgggcggccgcgcaacaaaggagtca
cccggcgatgagccccggcacccccggaccgaccatgggcagatcccagggcagcccaa
tggatccaatggtgatgaagagacctcagttgtatggcatgggcagtaaccctcattct
cagcctcagcagagcagtccgtacccaggaggttcctatggccctccaggcccacagcg
gtatccaattggcatccagggtcggactcccggggccatggccggaatgcagtaccctc
agcagcaggactctggagatgccacatggaaagaaacattctggttgatgccacctcag
tatggacagcaaggtgtgagtggttactgccagcagggccaacagccatattacagcca
gcagccgcagcccccgcacctcccaccccaggcgcagtatctgccgtcccagtcccagc
agaggtaccagccgcagcaggacatgtctcaggaaggctatggaactagatctcaacct
cctctggcccccggaaaacctaaccatgaagacttgaacttaatacagcaagaaagacc
atcaagtttaccagatctgtctggctccattgatgacctccccacgggaacggaagcaa
ctttgagctcagcagtcagtgcatccgggtccacgagcagccaaggggatcagagcaac
ccggcgcagtcgcctttctccccacatgcgtcccctcatctctccagcatcccgggggg
cccatctccctctcctgttggctctcctgtaggaagcaaccagtctcgatctggcccaa
tctctcctgcaagtatcccaggtagtcagatgcctccgcagccacccgggagccagtca
gaatccagttcccatcccgccttgagccagtcaccaatgccacaggaaagaggttttat
ggcaggcacacaaagaaaccctcagatggctcagtatggacctcaacagacaggaccat
ccatgtcgcctcatccttctcctgggggccagatgcatgctggaatcagtagctttcag
cagagtaactcaagtgggacttacggtccacagatgagccagtatggaccacaaggtaa
ctactccagacccccagcgtatagtggggtgcccagtgcaagctacagcggcccagggc
ccggtatgggtatcagtgccaacaaccagatgcatggacaagggccaagccagccatgt
ggtgctgtgcccctgggacgaatgccatcagctgggatgcagaacagaccatttcctgg
aaatatgagcagcatgacccccagttctcctggcatgtctcagcagggagggccaggaa
tggggccgccaatgccaactgtgaaccgtaaggcacaggaggcagccgcagcagtgatg
caggctgctgcgaactcagcacaaagcaggcaaggcagtttccccggcatgaaccagag
tggacttatggcttccagctctccctacagccagcccatgaacaacagctctagcctga
tgaacacgcaggcgccgccctacagcatggcgcccgccatggtgaacagctcggcagca
tctgtgggtcttgcagatatgatgtctcctggtgaatccaaactgcccctgcctctcaa
agcagacggcaaagaagaaggcactccacagcccgagagcaagtcaaagaagtccagct
cctccaccactactggggagaagatcacgaaggtgtacgagctggggaatgagccagag
agaaagctctgggtcgaccgatacctcaccttcatggaagagagaggctctcctgtctc
aagtctgcctgccgtgggcaagaagcccctggacctgttccgactctacgtctgcgtca
aagagatcgggggtttggcccaggttaataaaaacaagaagtggcgtgagctggcaacc
aacctaaacgttggcacctcaagcagtgcagcgagctccctgaaaaagcagtatattca
gtacctgtttgcctttgagtgcaagatcgaacgtggggaggagcccccgccggaagtct
tcagcaccggggacaccaaaaagcagcccaagctccagccgccatctcctgctaactcg
ggatccttgcaaggcccacagaccccccagtcaactggcagcaattccatggcagaggt
tccaggtgacctgaagccacctaccccagcctccacccctcacggccagatgactccaa
tgcaaggtggaagaagcagtacaatcagtgtgcacgacccattctcagatgtgagtgat
tcatccttcccgaaacggaactccatgactccaaacgccccctaccagcagggcatgag
catgcccgatgtgatgggcaggatgccctatgagcccaacaaggacccctttgggggaa
tgagaaaagtgcctggaagcagcgagccctttatgacgcaaggacagatgcccaacagc
agcatgcaggacatgtacaaccaaagtccctccggagcaatgtctaacctgggcatggg
gcagcgccagcagtttccctatggagccagttacgaccgaaggcatgaaccttatgggc
agcagtatccaggccaaggccctccctcgggacagccgccgtatggagggcaccagccc
ggcctgtacccacagcagccgaattacaaacgccatatggacggcatgtacgggccccc
agccaagcgccacgagggcgacatgtacaacatgcagtacagcagccagcagcaggaga
tgtacaaccagtatggaggctcctactcgggcccggaccgcaggcccatccagggccag
tacccgtatccctacagcagggagaggatgcagggcccggggcagatccagacacacgg
aatcccgcctcagatgatgggcggcccgctgcagtcgtcctccagtgaggggcctcagc
agaatatgtgggcagcacgcaatgatatgccttatccctaccagaacaggcagggccct
ggcggccctacacaggcgcccccttacccaggcatgaaccgcacagacgatatgatggt
acccgatcagaggataaatcatgagagccagtggccttctcacgtcagccagcgtcagc
cttatatgtcgtcctcagcctccatgcagcccatcacacgcccaccacagccgtcctac
cagacgccaccgtcactgccaaatcacatctccagggcgcccagcccagcgtccttcca
gcgctccctggagaaccgcatgtctccaagcaagtctccttttctgccgtctatgaaga
tgcagaaggtcatgcccacggtccccacatcccaggtcaccgggccaccaccccaacca
cccccaatcagaagggagatcacctttcctcctggctcagtagaagcatcacaaccagt
cttgaaacaaaggcgaaagattacctccaaagatatcgttactcctgaggcgtggcgtg
tgatgatgtcccttaaatcaggtcttttggctgagagtacgtgggctttggacactatt
aatattcttctgtatgatgacagcactgttgctactttcaatctctcccagttgtctgg
atttctcgaacttttagtcgagtactttagaaaatgcctgattgacatttttggaattc
ttatggaatatgaagtgggagaccccagccaaaaagcacttgatcacaacgcagcaagg
aaggatgacagccagtccttggcagacgattctgggaaagaggaggaagatgctgaatg
tattgatgacgacgaggaagacgaggaggatgaggaggaagacagcgagaagacagaaa
gcgatgaaaagagcagcatcgctctgactgccccggacgccgctgcagacccaaaggag
aagcccaagcaagccagtaagttcgacaagctgccaataaagatagtcaaaaagaacaa
cctgtttgttgttgaccgatctgacaagttggggcgtgtgcaggagttcaatagtggcc
ttctgcactggcagctcggcgggggtgacaccaccgagcacattcagactcactttgag
agcaagatggaaattcctcctcgcaggcgcccacctccccccttaagctccgcaggtag
aaagaaagagcaagaaggcaaaggcgactctgaagagcagcaagagaaaagcatcatag
caaccatcgatgacgtcctctctgctcggccaggggcattgcctgaagacgcaaaccct
gggccccagaccgaaagcagtaagtttccctttggtatccagcaagccaaaagtcaccg
gaacatcaagctgctggaggacgagcccaggagccgagacgagactcctctgtgtacca
tcgcgcactggcaggactcgctggctaagcgatgcatctgtgtgtccaatattgtccgt
agcttgtcattcgtgcctggcaatgatgccgaaatgtccaaacatccaggcctggtgct
gatcctggggaagctgattcttcttcaccacgagcatccagagagaaagcgagcaccgc
agacctatgagaaagaggaggatgaggacaagggggtggcctgcagcaaagatgagtgg
tggtgggactgcctcgaggtcttgagggataacacgttggtcacgttggccaacatttc
cgggcagctagacttgtctgcttacacggaaagcatctgcttgccaattttggatggct
tgctgcactggatggtgtgcccgtctgcagaggcacaagatccctttccaactgtggga
cccaactcggtcctgtcgcctcagagacttgtgctggagaccctctgtaaactcagtat
ccaggacaataatgtggacctgatcttggccactcctccatttagtcgtcaggagaaat
tctatgctacattagttaggtacgttggggatcgcaaaaacccagtctgtcgagaaatg
tccatggcgcttttatcgaaccttgcccaaggggacgcactagcagcaagggccatagc
tgtgcagaaaggaagcattggaaacttgataagcttcctagaggatggggtcacgatgg
cccagtaccagcagagccagcacaacctcatgcacatgcagcccccgcccctggaacca
cctagcgtagacatgatgtgcagggcggccaaggctttgctagccatggccagagtgga
cgaaaaccgctcggaattccttttgcacgagggccggttgctggatatctcgatatcag
ctgtcctgaactctctggttgcatctgtcatctgtgatgtactgtttcagattgggcag
ttatgacataagtgagaaggcaagcatgtgtgagtgaagattagagggtcacatataac
tggctgttttctgttcttgtttatccagcgtaggaagaaggaaaagaaaatctttgctc
ctctgccccattcactatttaccaattgggaattaaagaaataattaatttgaacagtt
atgaaattaatatttgctgtctgtgtgtataagtacatcctttggggttttttttttct
cttttttttaaccaaagttgctgtctagtgcattcaaaggtcactttttgttcttcaca
gatctttttaatgttctttcccatgttgtattgcatttttgggggaagcaaattgactt
taaagaaaaaagttgtggcaaaagatgctaagatgcgaaaatttcaccacactgagtca
aaaaggtgaaaaattatccatttcctatgcgttttactcctcagagaatgaaaaaaact
gcatcccatcaccca
aagttctgtgcaatagaaatttctacagatacaggtataggggctcaaggaggtatgtc
ggtcagtagtcaaaactatgaaatgatactggtttctccacaggaatatggttccatta
ggctgggagcaaaaacaatgttttttaagattgagaatacatacctgacaacgatccgg
aaactgctcctcaccactcccgtcatgcctgctgtcggcgtttgaccttccacgtgaca
gttcttcacaattcctttcatcattttttaaatattttttttactgcctatgggctgtg
atgtatatagaagttgtacattaaacataccctcatttttttcttttcttttttttttt
tttttttagtacaaagttttagtttctttttcatgatgtggtaactacgaagtgatggt
agatttaaataattttttatttttattttatatattttttcattagggccatatctcca
aaaaaagaaagaaaaaatacaaaaaacaaaaacaaaaaaaaaagagggtaatgtacaag
tttctgtatgtataaagtcatgctcgatttcaggagagcagctgatcacaatttgcttc
atgaatcaaggtgtggaaatggttatatatggattgatttagaaaatggttaccagtac
agtcaaaaaagagaaaatgaaaaaaatacaactaaaaggaagaaacacaacttcaaaga
tttttcagtgatgagaatccacatttgtatttcaagataatgtagtttaaaaaaaaaaa
aaagaaaaaaacttgatgtaaattcctccttttcctctggcttaatgaatatcatttat
tcagtataaaatct
ttatatgttccacatgttaagaataaatgtacattaaatcttgttaagcactgtgatgg
gtgttcttgaatactgttctagtttccttaaagtggtttcctagtaatcaagttattta
caagaaataggggaatgcagcagtgtattcacattataaaaccctacatttggaagaga
cctttaggggttacctactttagagtggggagcaacagtttgattttctcaaattactt
agctaattagtctttctttgaagcaattaactctaacgacattgaggtatgatcatttt
cagtatttatgggaggtggctgctgacccacttgaggtgagatctcagaagcttaactg
gcctgaaaatgtaacattctgccttttactaactccatcttagtttaatcaaagttcaa
tctattccttgtttcttctgtgtgcctcagagttattttgcatttagtttactccaccg
tgtataatatttatactgtgcaatgttaaaaaagaatctgttatattgtatgtggtgta
catagtgcaaagtgatgatttctatttcagggcatattatggttctcatattccttcct
acctggtgcacagtagctttttaatactagtcacttctaatttaaactttctcttcctg
ggtcattgactgttactgtgtaataatcgatttctttgaaactgctgcataattatgct
gttagtggacctctacctcttctcttccctctcccaatcacagtatactcagaatcccc
agcccctcgcatacattgtgtcggttcacattactcacagtaatatatggaagagttag
acaagaacatgcagttacagtcattgtgagacgtgactctccagtgtcacgaggaaaaa
aatcatcttttctgcaaacagtctctcatctgtcaactcccacattactgagtcaaaca
gtcttcttacataacaatgcaaccaaatatatgttgaattaaagacccatttataattc
tgctttaaatacatctgcttgctaagaacagatttcagtgctccaagcttcaaatatgg
agatttgtaagagggaattcaatattattctaatttctctcttacagagtacaaataaa
aggtgtatacaaactccgaacatatccagtattccaattcctttgtcaatcagaagagt
aaaataattaacaaaagactgttgttatggtttgcattgtaaccgatacgcagagtctg
accgttgggcaacaagtttttctatcctgatgcgcaacacagtctctagagactaatcc
aggaagactttagcctcctttccatattctcacccccgaatcaagatttacagaagccc
acgaagaatttacagcctgcttgagatcatcttgcctataaactgagttattgctttgt
cctaaaaattagtcggtttttttttttctatgaggcttttcagaaatttacaggatgcc
cagactttacatgtgtaccaaaaaaaaaaaaaagataaaaaataaaggtgcaaagaaag
tttagtattttggaatggtgctataaagttgaa
[ARID1B sequence-3]
SEQ ID NO: 18
aagccatgctgggcccgataggctcagctagtcgtgtatttacccatatccgggctaga
gaggaaaagagaaaagtttcatttaaacctgaactaaaaactttcaccatgaaagcaca
cagcaggagcaggcccagagcgtaaggcgtgcccggcccggcgctccggcggggcctgc
ggagggggagggggtcgcggcttcccggcgggccgcgtggatgcgcacaggaggggccg
cggcctgaaaagtgggggttattgtctccccccgccccccgcccggcctcgccacgccg
cggcgatcatggccgcgcgggcagcagcggcggcggcggcggcggcggcgcgggcgcgg
gcgcgggcaggcagcggcgaacggcgggcgccccccgggccgcggccggcgcccggagc
ccgggacctggaggcgggggcgcgcggcgcggcggcggcggcggcggcaccgggaccca
tgctggggggcggcggcgacggcggcggcggcctgaacagtgtgcaccaccaccccctg
ctcccccgtcacgaactcaacatggcccataacgcgggcgccgcggccgccgccggcac
ccacagcgccaagagcggcggctccgaggcggctctcaaggagggtggaagcgccgccg
cgctgtcctcctcctcctcctcctccgcggcggcagcggcggcatcctcttcctcctcg
tcgggcccgggctcggccatggagacggggctgctccccaaccacaaactgaaaaccgt
tggcgaagcccccgccgcgccgccccaccagcagcaccaccaccaccaccatgcccacc
accaccaccaccatgcccaccacctccaccaccaccacgcactacagcagcagctaaac
cagttccagcagcagcagcagcagcagcaacagcagcagcagcagcagcagcaacagca
acatcccatttccaacaacaacagcttgggcggcgcgggcggcggcgcgcctcagcccg
gccccgacatggagcagccgcaacatggaggcgccaaggacagtgctgcgggcggccag
gccgaccccccgggcccgccgctgctgagcaagccgggcgacgaggacgacgcgccgcc
caagatgggggagccggcgggcggccgctacgagcacccgggcttgggcgccctgggca
cgcagcagccgccggtcgccgtgcccgggggcggcggcggcccggcggccgtcccggag
tttaataattactatggcagcgctgcccctgcgagcggcggccccggcggccgcgctgg
gccttgctttgatcaacatggcggacaacaaagccccgggatggggatgatgcactccg
cctccgccgccgccgccggggcccccggcagcatggaccccctgcagaactcccacgaa
gggtaccccaacagccagtgcaaccattatccgggctacagccggcccggcgcgggcgg
cggcggcggcggcggcggcggaggaggaggaggcagcggaggaggaggaggaggaggag
gagcaggagcaggaggagcaggagcgggagctgtggcggcggcggccgcggcggcggcg
gcagcagcaggaggcggcggcggcggcggctatgggggctcgtccgcggggtacggggt
gctgagctccccccggcagcagggcggcggcatgatgatgggccccgggggcggcgggg
ccgcgagcctcagcaaggcggccgccggctcggcggcggggggcttccagcgcttcgcc
ggccagaaccagcacccgtcgggggccaccccgaccctcaatcagctgctcacctcgcc
cagccccatgatgcggagctacggcggcagctaccccgagtacagcagccccagcgcgc
cgccgccgccgccgtcgcagccccagtcccaggcggcggcggcgggggcggcggcgggc
ggccagcaggcggccgcgggcatgggcttgggcaaggacatgggcgcccagtacgccgc
tgccagcccggcctgggcggccgcgcaacaaaggagtcacccggcgatgagccccggca
cccccggaccgaccatgggcagatcccagggcagcccaatggatccaatggtgatgaag
agacctcagttgtatggcatgggcagtaaccctcattctcagcctcagcagagcagtcc
gtacccaggaggttcctatggccctccaggcccacagcggtatccaattggcatccagg
gtcggactcccggggccatggccggaatgcagtaccctcagcagcaggactctggagat
gccacatggaaagaaacattctggttgatgccacctcagtatggacagcaaggtgtgag
tggttactgccagcagggccaacagccatattacagccagcagccgcagcccccgcacc
tcccaccccaggcgcagtatctgccgtcccagtcccagcagaggtaccagccgcagcag
gacatgtctcaggaaggctatggaactagatctcaacctcctctggcccccggaaaacc
taaccatgaagacttgaacttaatacagcaagaaagaccatcaagtttaccagatctgt
ctggctccattgatgacctccccacgggaacggaagcaactttgagctcagcagtcagt
gcatccgggtccacgagcagccaaggggatcagagcaacccggcgcagtcgcctttctc
cccacatgcgtcccctcatctctccagcatcccggggggcccatctccctctcctgttg
gctctcctgtaggaagcaaccagtctcgatctggcccaatctctcctgcaagtatccca
ggtagtcagatgcctccgcagccacccgggagccagtcagaatccagttcccatcccgc
cttgagccagtcaccaatgccacaggaaagaggttttatggcaggcacacaaagaaacc
ctcagatggctcagtatggacctcaacagacaggaccatccatgtcgcctcatccttct
cctgggggccagatgcatgctggaatcagtagctttcagcagagtaactcaagtgggac
ttacggtccacagatgagccagtatggaccacaaggtaactactccagacccccagcgt
atagtggggtgcccagtgcaagctacagcggcccagggcccggtatgggtatcagtgcc
aacaaccagatgcatggacaagggccaagccagccatgtggtgctgtgcccctgggacg
aatgccatcagctgggatgcagaacagaccatttcctggaaatatgagcagcatgaccc
ccagttctcctggcatgtctcagcagggagggccaggaatggggccgccaatgccaact
gtgaaccgtaaggcacaggaggcagccgcagcagtgatgcaggctgctgcgaactcagc
acaaagcaggcaaggcagtttccccggcatgaaccagagtggacttatggcttccagct
ctccctacagccagcccatgaacaacagctctagcctgatgaacacgcaggcgccgccc
tacagcatggcgcccgccatggtgaacagctcggcagcatctgtgggtcttgcagatat
gatgtctcctggtgaatccaaactgcccctgcctctcaaagcagacggcaaagaagaag
gcactccacagcccgagagcaagtcaaagaagtccagctcctccaccactactggggag
aagatcacgaaggtgtacgagctggggaatgagccagagagaaagctctgggtcgaccg
atacctcaccttcatggaagagagaggctctcctgtctcaagtctgcctgccgtgggca
agaagcccctggacctgttccgactctacgtctgcgtcaaagagatcgggggtttggcc
caggttaataaaaacaagaagtggcgtgagctggcaaccaacctaaacgttggcacctc
aagcagtgcagcgagctccctgaaaaagcagtatattcagtacctgtttgcctttgagt
gcaagatcgaacgtggggaggagcccccgccggaagtcttcagcaccggggacaccaaa
aagcagcccaagctccagccgccatctcctgctaactcgggatccttgcaaggcccaca
gaccccccagtcaactggcagcaattccatggcagaggttccaggtgacctgaagccac
ctaccccagcctccacccctcacggccagatgactccaatgcaaggtggaagaagcagt
acaatcagtgtgcacgacccattctcagatgtgagtgattcatccttcccgaaacggaa
ctccatgactccaaacgccccctaccagcagggcatgagcatgcccgatgtgatgggca
ggatgccctatgagcccaacaaggacccctttgggggaatgagaaaagtgcctggaagc
agcgagccctttatgacgcaaggacagatgcccaacagcagcatgcaggacatgtacaa
ccaaagtccctccggagcaatgtctaacctgggcatggggcagcgccagcagtttccct
atggagccagttacgaccgaaggcatgaaccttatgggcagcagtatccaggccaaggc
cctccctcgggacagccgccgtatggagggcaccagcccggcctgtacccacagcagcc
gaattacaaacgccatatggacggcatgtacgggcccccagccaagcgccacgagggcg
acatgtacaacatgcagtacagcagccagcagcaggagatgtacaaccagtatggaggc
tcctactcgggcccggaccgcaggcccatccagggccagtacccgtatccctacagcag
ggagaggatgcagggcccggggcagatccagacacacggaatcccgcctcagatgatgg
gcggcccgctgcagtcgtcctccagtgaggggcctcagcagaatatgtgggcagcacgc
aatgatatgccttatccctaccagaacaggcagggccctggcggccctacacaggcgcc
cccttacccaggcatgaaccgcacagacgatatgatggtacccgatcagaggataaatc
atgagagccagtggccttctcacgtcagccagcgtcagccttatatgtcgtcctcagcc
tccatgcagcccatcacacgcccaccacagccgtcctaccagacgccaccgtcactgcc
aaatcacatctccagggcgcccagcccagcgtccttccagcgctccctggagaaccgca
tgtctccaagcaagtctccttttctgccgtctatgaagatgcagaaggtcatgcccacg
gtccccacatcccaggtcaccgggccaccaccccaaccacccccaatcagaagggagat
cacctttcctcctggctcagtagaagcatcacaaccagtcttgaaacaaaggcgaaaga
ttacctccaaagatatcgttactcctgaggcgtggcgtgtgatgatgtcccttaaatca
ggtcttttggctgagagtacgtgggctttggacactattaatattcttctgtatgatga
cagcactgttgctactttcaatctctcccagttgtctggatttctcgaacttttagtcg
agtactttagaaaatgcctgattgacatttttggaattcttatggaatatgaagtggga
gaccccagccaaaaagcacttgatcacaacgcagcaaggaaggatgacagccagtcctt
ggcagacgattctgggaaagaggaggaagatgctgaatgtattgatgacgacgaggaag
acgaggaggatgaggaggaagacagcgagaagacagaaagcgatgaaaagagcagcatc
gctctgactgccccggacgccgctgcagacccaaaggagaagcccaagcaagccagtaa
gttcgacaagctgccaataaagatagtcaaaaagaacaacctgtttgttgttgaccgat
ctgacaagttggggcgtgtgcaggagttcaatagtggccttctgcactggcagctcggc
gggggtgacaccaccgagcacattcagactcactttgagagcaagatggaaattcctcc
tcgcaggcgcccacctccccccttaagctccgcaggtagaaagaaagagcaagaaggca
aaggcgactctgaagagcagcaagagaaaagcatcatagcaaccatcgatgacgtcctc
tctgctcggccaggggcattgcctgaagacgcaaaccctgggccccagaccgaaagcag
taagtttccctttggtatccagcaagccaaaagtcaccggaacatcaagctgctggagg
acgagcccaggagccgagacgagactcctctgtgtaccatcgcgcactggcaggactcg
ctggctaagcgatgcatctgtgtgtccaatattgtccgtagcttgtcattcgtgcctgg
caatgatgccgaaatgtccaaacatccaggcctggtgctgatcctggggaagctgattc
ttcttcaccacgagcatccagagagaaagcgagcaccgcagacctatgagaaagaggag
gatgaggacaagggggtggcctgcagcaaagatgagtggtggtgggactgcctcgaggt
cttgagggataacacgttggtcacgttggccaacatttccgggcagctagacttgtctg
cttacacggaaagcatctgcttgccaattttggatggcttgctgcactggatggtgtgc
ccgtctgcagaggcacaagatccctttccaactgtgggacccaactcggtcctgtcgcc
tcagagacttgtgctggagaccctctgtaaactcagtatccaggacaataatgtggacc
tgatcttggccactcctccatttagtcgtcaggagaaattctatgctacattagttagg
tacgttggggatcgcaaaaacccagtctgtcgagaaatgtccatggcgcttttatcgaa
ccttgcccaaggggacgcactagcagcaagggccatagctgtgcagaaaggaagcattg
gaaacttgataagcttcctagaggatggggtcacgatggcccagtaccagcagagccag
cacaacctcatgcacatgcagcccccgcccctggaaccacctagcgtagacatgatgtg
cagggcggccaaggctttgctagccatggccagagtggacgaaaaccgctcggaattcc
ttttgcacgagggccggttgctggatatctcgatatcagctgtcctgaactctctggtt
gcatctgtcatctgtgatgtactgtttcagattgggcagttatgacataagtgagaagg
caagcatgtgtgagtgaagattagagggtcacatataactggctgttttctgttcttgt
ttatccagcgtaggaagaaggaaaagaaaatctttgctcctctgccccattcactattt
accaattgggaattaaagaaataattaatttgaacagttatgaaattaatatttgctgt
ctgtgtgtataagtacatcctttggggttttttttttctcttttttttaaccaaagttg
ctgtctagtgcattcaaaggtcactttttgttcttcacagatctttttaatgttctttc
ccatgttgtattgcatttttgggggaagcaaattgactttaaagaaaaaagttgtggca
aaagatgctaagatgcgaaaatttcaccacactgagtcaaaaaggtgaaaaattatcca
tttcctatgcgttttactcctcagagaatgaaaaaaactgcatcccatcacccaaagtt
ctgtgcaatagaaatttctacagatacaggtataggggctcaaggaggtatgtcggtca
gtagtcaaaactatgaaatgatactggtttctccacaggaatatggttccattaggctg
ggagcaaaaacaatgttttttaagattgagaatacatacctgacaacgatccggaaact
gctcctcaccactcccgtcatgcctgctgtcggcgtttgaccttccacgtgacagttct
tcacaattcctttcatcattttttaaatattttttttactgcctatgggctgtgatgta
tatagaagttgtacattaaacataccctcatttttttcttttctttttttttttttttt
ttagtacaaagttttagtttctttttcatgatgtggtaactacgaagtgatggtagatt
taaataattttttatttttattttatatattttttcattagggccatatctccaaaaaa
agaaagaaaaaatacaaaaaacaaaaacaaaaaaaaaagagggtaatgtacaagtttct
gtatgtataaagtcatgctcgatttcaggagagcagctgatcacaatttgcttcatgaa
tcaaggtgtggaaatggttatatatggattgatttagaaaatggttaccagtacagtca
aaaaagagaaaatgaaaaaaatacaactaaaaggaagaaacacaacttcaaagattttt
cagtgatgagaatccacatttgtatttcaagataatgtagtttaaaaaaaaaaaaaaga
aaaaaacttgatgtaaattcctccttttcctctggcttaatgaatatcatttattcagt
ataaaatctttatatgttccacatgttaagaataaatgtacattaaatcttgttaagca
ctgtgatgggtgttcttgaatactgttctagtttccttaaagtggtttcctagtaatca
agttatttacaagaaataggggaatgcagcagtgtattcacattataaaaccctacatt
tggaagagacctttaggggttacctactttagagtggggagcaacagtttgattttctc
aaattacttagctaattagtctttctttgaagcaattaactctaacgacattgaggtat
gatcattttcagtatttatgggaggtggctgctgacccacttgaggtgagatctcagaa
gcttaactggcctgaaaatgtaacattctgccttttactaactccatcttagtttaatc
aaagttcaatctattccttgtttcttctgtgtgcctcagagttattttgcatttagttt
actccaccgtgtataatatttatactgtgcaatgttaaaaaagaatctgttatattgta
tgtggtgtacatagtgcaaagtgatgatttctatttcagggcatattatggttctcata
ttccttcctacctggtgcacagtagctttttaatactagtcacttctaatttaaacttt
ctcttcctgggtcattgactgttactgtgtaataatcgatttctttgaaactgctgcat
aattatgctgttagtggacctctacctcttctcttccctctcccaatcacagtatactc
agaatccccagcccctcgcatacattgtgtcggttcacattactcacagtaatatatgg
aagagttagacaagaacatgcagttacagtcattgtgagacgtgactctccagtgtcac
gaggaaaaaaatcatcttttctgcaaacagtctctcatctgtcaactcccacattactg
agtcaaacagtcttcttacataacaatgcaaccaaatatatgttgaattaaagacccat
ttataattctgctttaaatacatctgcttgctaagaacagatttcagtgctccaagctt
caaatatggagatttgtaagagggaattcaatattattctaatttctctcttacagagt
acaaataaaaggtgtatacaaactccgaacatatccagtattccaattcctttgtcaat
cagaagagtaaaataattaacaaaagactgttgttatggtttgcattgtaaccgatacg
cagagtctgaccgttgggcaacaagtttttctatcctgatgcgcaacacagtctctaga
gactaatccaggaagactttagcctcctttccatattctcacccccgaatcaagattta
Cagaagcccacgaagaatttacagcctgcttgagatcatcttgcctataaactgagtta
ttgctttgtcctaaaaattagtcggtttttttttttctatgaggcttttcagaaattta
caggatgcccagactttacatgtgtaccaaaaaaaaaaaaaagataaaaaataaaggtg
caaagaaagtttagtattttggaatggtgctataaagttgaa
[ARID1B sequence-4]
SEQ ID NO: 19
aagccatgctgggcccgataggctcagctagtcgtgtatttacccatatccgggctaga
gaggaaaagagaaaagtttcatttaaacctgaactaaaaactttcaccatgaaagcaca
cagcaggagcaggcccagagcgtaaggcgtgcccggcccggcgctccggcggggcctgc
ggagggggagggggtcgcggcttcccggcgggccgcgtggatgcgcacaggaggggccg
cggcctgaaaagtgggggttattgtctccccccgccccccgcccggcctcgccacgccg
cggcgatcatggccgcgcgggcagcagcggcggcggcggcggcggcggcgcgggcgcgg
gcgcgggcaggcagcggcgaacggcgggcgccccccgggccgcggccggcgcccggagc
ccgggacctggaggcgggggcgcgcggcgcggcggcggcggcggcggcaccgggaccca
tgctggggggcggcggcgacggcggcggcggcctgaacagtgtgcaccaccaccccctg
ctcccccgtcacgaactcaacatggcccataacgcgggcgccgcggccgccgccggcac
ccacagcgccaagagcggcggctccgaggcggctctcaaggagggtggaagcgccgccg
cgctgtcctcctcctcctcctcctccgcggcggcagcggcggcatcctcttcctcctcg
tcgggcccgggctcggccatggagacggggctgctccccaaccacaaactgaaaaccgt
tggcgaagcccccgccgcgccgccccaccagcagcaccaccaccaccaccatgcccacc
accaccaccaccatgcccaccacctccaccaccaccacgcactacagcagcagctaaac
cagttccagcagcagcagcagcagcagcaacagcagcagcagcagcagcagcaacagca
acatcccatttccaacaacaacagcttgggcggcgcgggcggcggcgcgcctcagcccg
gccccgacatggagcagccgcaacatggaggcgccaaggacagtgctgcgggcggccag
gccgaccccccgggcccgccgctgctgagcaagccgggcgacgaggacgacgcgccgcc
caagatgggggagccggcgggcggccgctacgagcacccgggcttgggcgccctgggca
cgcagcagccgccggtcgccgtgcccgggggcggcggcggcccggcggccgtcccggag
tttaataattactatggcagcgctgcccctgcgagcggcggccccggcggccgcgctgg
gccttgctttgatcaacatggcggacaacaaagccccgggatggggatgatgcactccg
cctccgccgccgccgccggggcccccggcagcatggaccccctgcagaactcccacgaa
gggtaccccaacagccagtgcaaccattatccgggctacagccggcccggcgcgggcgg
cggcggcggcggcggcggcggaggaggaggaggcagcggaggaggaggaggaggaggag
gagcaggagcaggaggagcaggagcgggagctgtggcggcggcggccgcggcggcggcg
gcagcagcaggaggcggcggcggcggcggctatgggggctcgtccgcggggtacggggt
gctgagctccccccggcagcagggcggcggcatgatgatgggccccgggggcggcgggg
ccgcgagcctcagcaaggcggccgccggctcggcggcggggggcttccagcgcttcgcc
ggccagaaccagcacccgtcgggggccaccccgaccctcaatcagctgctcacctcgcc
cagccccatgatgcggagctacggcggcagctaccccgagtacagcagccccagcgcgc
cgccgccgccgccgtcgcagccccagtcccaggcggcggcggcgggggcggcggcgggc
ggccagcaggcggccgcgggcatgggcttgggcaaggacatgggcgcccagtacgccgc
tgccagcccggcctgggcggccgcgcaacaaaggagtcacccggcgatgagccccggca
cccccggaccgaccatgggcagatcccagggcagcccaatggatccaatggtgatgaag
agacctcagttgtatggcatgggcagtaaccctcattctcagcctcagcagagcagtcc
gtacccaggaggttcctatggccctccaggcccacagcggtatccaattggcatccagg
gtcggactcccggggccatggccggaatgcagtaccctcagcagcagatgccacctcag
tatggacagcaaggtgtgagtggttactgccagcagggccaacagccatattacagcca
gcagccgcagcccccgcacctcccaccccaggcgcagtatctgccgtcccagtcccagc
agaggtaccagccgcagcaggacatgtctcaggaaggctatggaactagatctcaacct
cctctggcccccggaaaacctaaccatgaagacttgaacttaatacagcaagaaagacc
atcaagtttaccagatctgtctggctccattgatgacctccccacgggaacggaagcaa
ctttgagctcagcagtcagtgcatccgggtccacgagcagccaaggggatcagagcaac
ccggcgcagtcgcctttctccccacatgcgtcccctcatctctccagcatcccgggggg
cccatctccctctcctgttggctctcctgtaggaagcaaccagtctcgatctggcccaa
tctctcctgcaagtatcccaggtagtcagatgcctccgcagccacccgggagccagtca
gaatccagttcccatcccgccttgagccagtcaccaatgccacaggaaagaggttttat
ggcaggcacacaaagaaaccctcagatggctcagtatggacctcaacagacaggaccat
ccatgtcgcctcatccttctcctgggggccagatgcatgctggaatcagtagctttcag
cagagtaactcaagtgggacttacggtccacagatgagccagtatggaccacaaggtaa
ctactccagacccccagcgtatagtggggtgcccagtgcaagctacagcggcccagggc
ccggtatgggtatcagtgccaacaaccagatgcatggacaagggccaagccagccatgt
ggtgctgtgcccctgggacgaatgccatcagctgggatgcagaacagaccatttcctgg
aaatatgagcagcatgacccccagttctcctggcatgtctcagcagggagggccaggaa
tggggccgccaatgccaactgtgaaccgtaaggcacaggaggcagccgcagcagtgatg
caggctgctgcgaactcagcacaaagcaggcaaggcagtttccccggcatgaaccagag
tggacttatggcttccagctctccctacagccagcccatgaacaacagctctagcctga
tgaacacgcaggcgccgccctacagcatggcgcccgccatggtgaacagctcggcagca
tctgtgggtcttgcagatatgatgtctcctggtgaatccaaactgcccctgcctctcaa
agcagacggcaaagaagaaggcactccacagcccgagagcaagtcaaaggatagctaca
gctctcagggtatttctcagcccccaaccccaggcaacctgccagtcccttccccaatg
tcccccagctctgctagcatctcctcatttcatggagatgaaagtgatagcattagcag
cccaggctggccaaagactccatcaagccctaagtccagctcctccaccactactgggg
agaagatcacgaaggtgtacgagctggggaatgagccagagagaaagctctgggtcgac
cgatacctcaccttcatggaagagagaggctctcctgtctcaagtctgcctgccgtggg
caagaagcccctggacctgttccgactctacgtctgcgtcaaagagatcgggggtttgg
cccaggttaataaaaacaagaagtggcgtgagctggcaaccaacctaaacgttggcacc
tcaagcagtgcagcgagctccctgaaaaagcagtatattcagtacctgtttgcctttga
gtgcaagatcgaacgtggggaggagcccccgccggaagtcttcagcaccggggacacca
aaaagcagcccaagctccagccgccatctcctgctaactcgggatccttgcaaggccca
cagaccccccagtcaactggcagcaattccatggcagaggttccaggtgacctgaagcc
acctaccccagcctccacccctcacggccagatgactccaatgcaaggtggaagaagca
gtacaatcagtgtgcacgacccattctcagatgtgagtgattcatccttcccgaaacgg
aactccatgactccaaacgccccctaccagcagggcatgagcatgcccgatgtgatggg
caggatgccctatgagcccaacaaggacccctttgggggaatgagaaaagtgcctggaa
gcagcgagccctttatgacgcaaggacagatgcccaacagcagcatgcaggacatgtac
aaccaaagtccctccggagcaatgtctaacctgggcatggggcagcgccagcagtttcc
ctatggagccagttacgaccgaaggcatgaaccttatgggcagcagtatccaggccaag
gccctccctcgggacagccgccgtatggagggcaccagcccggcctgtacccacagcag
ccgaattacaaacgccatatggacggcatgtacgggcccccagccaagcgccacgaggg
cgacatgtacaacatgcagtacagcagccagcagcaggagatgtacaaccagtatggag
gctcctactcgggcccggaccgcaggcccatccagggccagtacccgtatccctacagc
agggagaggatgcagggcccggggcagatccagacacacggaatcccgcctcagatgat
gggcggcccgctgcagtcgtcctccagtgaggggcctcagcagaatatgtgggcagcac
gcaatgatatgccttatccctaccagaacaggcagggccctggcggccctacacaggcg
cccccttacccaggcatgaaccgcacagacgatatgatggtacccgatcagaggataaa
tcatgagagccagtggccttctcacgtcagccagcgtcagccttatatgtcgtcctcag
cctccatgcagcccatcacacgcccaccacagccgtcctaccagacgccaccgtcactg
ccaaatcacatctccagggcgcccagcccagcgtccttccagcgctccctggagaaccg
catgtctccaagcaagtctccttttctgccgtctatgaagatgcagaaggtcatgccca
cggtccccacatcccaggtcaccgggccaccaccccaaccacccccaatcagaagggag
atcacctttcctcctggctcagtagaagcatcacaaccagtcttgaaacaaaggcgaaa
gattacctccaaagatatcgttactcctgaggcgtggcgtgtgatgatgtcccttaaat
caggtcttttggctgagagtacgtgggctttggacactattaatattcttctgtatgat
gacagcactgttgctactttcaatctctcccagttgtctggatttctcgaacttttagt
cgagtactttagaaaatgcctgattgacatttttggaattcttatggaatatgaagtgg
gagaccccagccaaaaagcacttgatcacaacgcagcaaggaaggatgacagccagtcc
ttggcagacgattctgggaaagaggaggaagatgctgaatgtattgatgacgacgagga
agacgaggaggatgaggaggaagacagcgagaagacagaaagcgatgaaaagagcagca
tcgctctgactgccccggacgccgctgcagacccaaaggagaagcccaagcaagccagt
aagttcgacaagctgccaataaagatagtcaaaaagaacaacctgtttgttgttgaccg
atctgacaagttggggcgtgtgcaggagttcaatagtggccttctgcactggcagctcg
gcgggggtgacaccaccgagcacattcagactcactttgagagcaagatggaaattcct
cctcgcaggcgcccacctccccccttaagctccgcaggtagaaagaaagagcaagaagg
caaaggcgactctgaagagcagcaagagaaaagcatcatagcaaccatcgatgacgtcc
tctctgctcggccaggggcattgcctgaagacgcaaaccctgggccccagaccgaaagc
agtaagtttccctttggtatccagcaagccaaaagtcaccggaacatcaagctgctgga
ggacgagcccaggagccgagacgagactcctctgtgtaccatcgcgcactggcaggact
cgctggctaagcgatgcatctgtgtgtccaatattgtccgtagcttgtcattcgtgcct
ggcaatgatgccgaaatgtccaaacatccaggcctggtgctgatcctggggaagctgat
tcttcttcaccacgagcatccagagagaaagcgagcaccgcagacctatgagaaagagg
aggatgaggacaagggggtggcctgcagcaaagatgagtggtggtgggactgcctcgag
gtcttgagggataacacgttggtcacgttggccaacatttccgggcagctagacttgtc
tgcttacacggaaagcatctgcttgccaattttggatggcttgctgcactggatggtgt
gcccgtctgcagaggcacaagatccctttccaactgtgggacccaactcggtcctgtcg
cctcagagacttgtgctggagaccctctgtaaactcagtatccaggacaataatgtgga
cctgatcttggccactcctccatttagtcgtcaggagaaattctatgctacattagtta
ggtacgttggggatcgcaaaaacccagtctgtcgagaaatgtccatggcgcttttatcg
aaccttgcccaaggggacgcactagcagcaagggccatagctgtgcagaaaggaagcat
tggaaacttgataagcttcctagaggatggggtcacgatggcccagtaccagcagagcc
agcacaacctcatgcacatgcagcccccgcccctggaaccacctagcgtagacatgatg
tgcagggcggccaaggctttgctagccatggccagagtggacgaaaaccgctcggaatt
ccttttgcacgagggccggttgctggatatctcgatatcagctgtcctgaactctctgg
ttgcatctgtcatctgtgatgtactgtttcagattgggcagttatgacataagtgagaa
ggcaagcatgtgtgagtgaagattagagggtcacatataactggctgttttctgttctt
gtttatccagcgtaggaagaaggaaaagaaaatctttgctcctctgccccattcactat
ttaccaattgggaattaaagaaataattaatttgaacagttatgaaattaatatttgct
gtctgtgtgtataagtacatcctttggggttttttttttctcttttttttaaccaaagt
tgctgtctagtgcattcaaaggtcactttttgttcttcacagatctttttaatgttctt
tcccatgttgtattgcatttttgggggaagcaaattgactttaaagaaaaaagttgtgg
caaaagatgctaagatgcgaaaatttcaccacactgagtcaaaaaggtgaaaaattatc
catttcctatgcgttttactcctcagagaatgaaaaaaactgcatcccatcacccaaag
ttctgtgcaatagaaatttctacagatacaggtataggggctcaaggaggtatgtcggt
cagtagtcaaaactatgaaatgatactggtttctccacaggaatatggttccattaggc
tgggagcaaaaacaatgttttttaagattgagaatacatacctgacaacgatccggaaa
ctgctcctcaccactcccgtcatgcctgctgtcggcgtttgaccttccacgtgacagtt
cttcacaattcctttcatcattttttaaatattttttttactgcctatgggctgtgatg
tatatagaagttgtacattaaacataccctcatttttttcttttctttttttttttttt
ttttagtacaaagttttagtttctttttcatgatgtggtaactacgaagtgatggtaga
tttaaataattttttatttttattttatatattttttcattagggccatatctccaaaa
aaagaaagaaaaaatacaaaaaacaaaaacaaaaaaaaaagagggtaatgtacaagttt
ctgtatgtataaagtcatgctcgatttcaggagagcagctgatcacaatttgcttcatg
aatcaaggtgtggaaatggttatatatggattgatttagaaaatggttaccagtacagt
Caaaaaagagaaaatgaaaaaaatacaactaaaaggaagaaacacaacttcaaagattt
ttcagtgatgagaatccacatttgtatttcaagataatgtagtttaaaaaaaaaaaaaa
gaaaaaaacttgatgtaaattcctccttttcctctggcttaatgaatatcatttattca
gtataaaatctttatatgttccacatgttaagaataaatgtacattaaatcttgttaag
cactgtgatgggtgttcttgaatactgttctagtttccttaaagtggtttcctagtaat
caagttatttacaagaaataggggaatgcagcagtgtattcacattataaaaccctaca
tttggaagagacctttaggggttacctactttagagtggggagcaacagtttgattttc
tcaaattacttagctaattagtctttctttgaagcaattaactctaacgacattgaggt
atgatcattttcagtatttatgggaggtggctgctgacccacttgaggtgagatctcag
aagcttaactggcctgaaaatgtaacattctgccttttactaactccatcttagtttaa
tcaaagttcaatctattccttgtttcttctgtgtgcctcagagttattttgcatttagt
ttactccaccgtgtataatatttatactgtgcaatgttaaaaaagaatctgttatattg
tatgtggtgtacatagtgcaaagtgatgatttctatttcagggcatattatggttctca
tattccttcctacctggtgcacagtagctttttaatactagtcacttctaatttaaact
ttctcttcctgggtcattgactgttactgtgtaataatcgatttctttgaaactgctgc
ataattatgctgttagtggacctctacctcttctcttccctctcccaatcacagtatac
tcagaatccccagcccctcgcatacattgtgtcggttcacattactcacagtaatatat
ggaagagttagacaagaacatgcagttacagtcattgtgagacgtgactctccagtgtc
acgaggaaaaaaatcatcttttctgcaaacagtctctcatctgtcaactcccacattac
tgagtcaaacagtcttcttacataacaatgcaaccaaatatatgttgaattaaagaccc
atttataattctgctttaaatacatctgcttgctaagaacagatttcagtgctccaagc
ttcaaatatggagatttgtaagagggaattcaatattattctaatttctctcttacaga
gtacaaataaaaggtgtatacaaactccgaacatatccagtattccaattcctttgtca
atcagaagagtaaaataattaacaaaagactgttgttatggtttgcattgtaaccgata
cgcagagtctgaccgttgggcaacaagtttttctatcctgatgcgcaacacagtctcta
gagactaatccaggaagactttagcctcctttccatattctcacccccgaatcaagatt
tacagaagcccacgaagaatttacagcctgcttgagatcatcttgcctataaactgagt
tattgctttgtcctaaaaattagtcggtttttttttttctatgaggcttttcagaaatt
tacaggatgcccagactttacatgtgtaccaaaa
aaaaaaaaaagataaaaaataaaggtgcaaagaaagtttagtattttggaatggtgcta
taaagttgaa
[ARID1B sequence-5]
SEQ ID NO: 20
aagccatgctgggcccgataggctcagctagtcgtgtatttacccatatccgggctaga
gaggaaaagagaaaagtttcatttaaacctgaactaaaaactttcaccatgaaagcaca
cagcaggagcaggcccagagcgtaaggcgtgcccggcccggcgctccggcggggcctgc
ggagggggagggggtcgcggcttcccggcgggccgcgtggatgcgcacaggaggggccg
cggcctgaaaagtgggggttattgtctccccccgccccccgcccggcctcgccacgccg
cggcgatcatggccgcgcgggcagcagcggcggcggcggcggcggcggcgcgggcgcgg
gcgcgggcaggcagcggcgaacggcgggcgccccccgggccgcggccggcgcccggagc
ccgggacctggaggcgggggcgcgcggcgcggcggcggcggcggcggcaccgggaccca
tgctggggggcggcggcgacggcggcggcggcctgaacagtgtgcaccaccaccccctg
ctcccccgtcacgaactcaacatggcccataacgcgggcgccgcggccgccgccggcac
ccacagcgccaagagcggcggctccgaggcggctctcaaggagggtggaagcgccgccg
cgctgtcctcctcctcctcctcctccgcggcggcagcggcggcatcctcttcctcctcg
tcgggcccgggctcggccatggagacggggctgctccccaaccacaaactgaaaaccgt
tggcgaagcccccgccgcgccgccccaccagcagcaccaccaccaccaccatgcccacc
accaccaccaccatgcccaccacctccaccaccaccacgcactacagcagcagctaaac
cagttccagcagcagcagcagcagcagcaacagcagcagcagcagcagcagcaacagca
acatcccatttccaacaacaacagcttgggcggcgcgggcggcggcgcgcctcagcccg
gccccgacatggagcagccgcaacatggaggcgccaaggacagtgctgcgggcggccag
gccgaccccccgggcccgccgctgctgagcaagccgggcgacgaggacgacgcgccgcc
caagatgggggagccggcgggcggccgctacgagcacccgggcttgggcgccctgggca
cgcagcagccgccggtcgccgtgcccgggggcggcggcggcccggcggccgtcccggag
tttaataattactatggcagcgctgcccctgcgagcggcggccccggcggccgcgctgg
gccttgctttgatcaacatggcggacaacaaagccccgggatggggatgatgcactccg
cctccgccgccgccgccggggcccccggcagcatggaccccctgcagaactcccacgaa
gggtaccccaacagccagtgcaaccattatccgggctacagccggcccggcgcgggcgg
cggcggcggcggcggcggcggaggaggaggaggcagcggaggaggaggaggaggaggag
gagcaggagcaggaggagcaggagcgggagctgtggcggcggcggccgcggcggcggcg
gcagcagcaggaggcggcggcggcggcggctatgggggctcgtccgcggggtacggggt
gctgagctccccccggcagcagggcggcggcatgatgatgggccccgggggcggcgggg
ccgcgagcctcagcaaggcggccgccggctcggcggcggggggcttccagcgcttcgcc
ggccagaaccagcacccgtcgggggccaccccgaccctcaatcagctgctcacctcgcc
cagccccatgatgcggagctacggcggcagctaccccgagtacagcagccccagcgcgc
cgccgccgccgccgtcgcagccccagtcccaggcggcggcggcgggggcggcggcgggc
ggccagcaggcggccgcgggcatgggcttgggcaaggacatgggcgcccagtacgccgc
tgccagcccggcctgggcggccgcgcaacaaaggagtcacccggcgatgagccccggca
cccccggaccgaccatgggcagatcccagggcagcccaatggatccaatggtgatgaag
agacctcagttgtatggcatgggcagtaaccctcattctcagcctcagcagagcagtcc
gtacccaggaggttcctatggccctccaggcccacagcggtatccaattggcatccagg
gtcggactcccggggccatggccggaatgcagtaccctcagcagcagatgccacctcag
tatggacagcaaggtgtgagtggttactgccagcagggccaacagccatattacagcca
gcagccgcagcccccgcacctcccaccccaggcgcagtatctgccgtcccagtcccagc
agaggtaccagccgcagcaggacatgtctcaggaaggctatggaactagatctcaacct
cctctggcccccggaaaacctaaccatgaagacttgaacttaatacagcaagaaagacc
atcaagtttaccagatctgtctggctccattgatgacctccccacgggaacggaagcaa
ctttgagctcagcagtcagtgcatccgggtccacgagcagccaaggggatcagagcaac
ccggcgcagtcgcctttctccccacatgcgtcccctcatctctccagcatcccgggggg
cccatctccctctcctgttggctctcctgtaggaagcaaccagtctcgatctggcccaa
tctctcctgcaagtatcccaggtagtcagatgcctccgcagccacccgggagccagtca
gaatccagttcccatcccgccttgagccagtcaccaatgccacaggaaagaggttttat
ggcaggcacacaaagaaaccctcagatggctcagtatggacctcaacagacaggaccat
ccatgtcgcctcatccttctcctgggggccagatgcatgctggaatcagtagctttcag
cagagtaactcaagtgggacttacggtccacagatgagccagtatggaccacaaggtaa
ctactccagacccccagcgtatagtggggtgcccagtgcaagctacagcggcccagggc
ccggtatgggtatcagtgccaacaaccagatgcatggacaagggccaagccagccatgt
ggtgctgtgcccctgggacgaatgccatcagctgggatgcagaacagaccatttcctgg
aaatatgagcagcatgacccccagttctcctggcatgtctcagcagggagggccaggaa
tggggccgccaatgccaactgtgaaccgtaaggcacaggaggcagccgcagcagtg
atgcaggctgctgcgaactcagcacaaagcaggcaaggcagtttccccggcatgaacca
gagtggacttatggcttccagctctccctacagccagcccatgaacaacagctctagcc
tgatgaacacgcaggcgccgccctacagcatggcgcccgccatggtgaacagctcggca
gcatctgtgggtcttgcagatatgatgtctcctggtgaatccaaactgcccctgcctct
caaagcagacggcaaagaagaaggcactccacagcccgagagcaagtcaaagaagtcca
gctcctccaccactactggggagaagatcacgaaggtgtacgagctggggaatgagcca
gagagaaagctctgggtcgaccgatacctcaccttcatggaagagagaggctctcctgt
ctcaagtctgcctgccgtgggcaagaagcccctggacctgttccgactctacgtctgcg
tcaaagagatcgggggtttggcccaggttaataaaaacaagaagtggcgtgagctggca
accaacctaaacgttggcacctcaagcagtgcagcgagctccctgaaaaagcagtatat
tcagtacctgtttgcctttgagtgcaagatcgaacgtggggaggagcccccgccggaag
tcttcagcaccggggacaccaaaaagcagcccaagctccagccgccatctcctgctaac
tcgggatccttgcaaggcccacagaccccccagtcaactggcagcaattccatggcaga
ggttccaggtgacctgaagccacctaccccagcctccacccctcacggccagatgactc
caatgcaaggtgga
agaagcagtacaatcagtgtgcacgacccattctcagatgtgagtgattcatccttccc
gaaacggaactccatgactccaaacgccccctaccagcagggcatgagcatgcccgatg
tgatgggcaggatgccctatgagcccaacaaggacccctttgggggaatgagaaaagtg
cctggaagcagcgagccctttatgacgcaaggacagatgcccaacagcagcatgcagga
catgtacaaccaaagtccctccggagcaatgtctaacctgggcatggggcagcgccagc
agtttccctatggagccagttacgaccgaaggcatgaaccttatgggcagcagtatcca
ggccaaggccctccctcgggacagccgccgtatggagggcaccagcccggcctgtaccc
acagcagccgaattacaaacgccatatggacggcatgtacgggcccccagccaagcgcc
acgagggcgacatgtacaacatgcagtacagcagccagcagcaggagatgtacaaccag
tatggaggctcctactcgggcccggaccgcaggcccatccagggccagtacccgtatcc
ctacagcagggagaggatgcagggcccggggcagatccagacacacggaatcccgcctc
agatgatgggcggcccgctgcagtcgtcctccagtgaggggcctcagcagaatatgtgg
gcagcacgcaatgatatgccttatccctaccagaacaggcagggccctggcggccctac
acaggcgcccccttacccaggcatgaaccgcacagacgatatgatggtacccgatcaga
ggataaatcatgagagccagtggccttctcacgtcagccagcgtcagccttatatgtcg
tcctcagcctccatgcagcccatcacacgcccaccacagccgtcctaccagacgccacc
gtcactgccaaatcacatctccagggcgcccagcccagcgtccttccagcgctccctgg
agaaccgcatgtctccaagcaagtctccttttctgccgtctatgaagatgcagaaggtc
atgcccacggtccccacatcccaggtcaccgggccaccaccccaaccacccccaatcag
aagggagatcacctttcctcctggctcagtagaagcatcacaaccagtcttgaaacaaa
ggcgaaagattacctccaaagatatcgttactcctgaggcgtggcgtgtgatgatgtcc
cttaaatcaggtcttttggctgagagtacgtgggctttggacactattaatattcttct
gtatgatgacagcactgttgctactttcaatctctcccagttgtctggatttctcgaac
ttttagtcgagtactttagaaaatgcctgattgacatttttggaattcttatggaatat
gaagtgggagaccccagccaaaaagcacttgatcacaacgcagcaaggaaggatgacag
ccagtccttggcagacgattctgggaaagaggaggaagatgctgaatgtattgatgacg
acgaggaagacgaggaggatgaggaggaagacagcgagaagacagaaagcgatgaaaag
agcagcatcgctctgactgccccggacgccgctgcagacccaaaggagaagcccaagca
agccagtaagttcgacaagctgccaataaagatagtcaaaaagaacaacctgtttgttg
ttgaccgatctgacaagttggggcgtgtgcaggagttcaatagtggccttctgcactgg
cagctcggcgggggtgacaccaccgagcacattcagactcactttgagagcaagatgga
aattcctcctcgcaggcgcccacctccccccttaagctccgcaggtagaaagaaagagc
aagaaggcaaaggcgactctgaagagcagcaagagaaaagcatcatagcaaccatcgat
gacgtcctctctgctcggccaggggcattgcctgaagacgcaaaccctgggccccagac
cgaaagcagtaagtttccctttggtatccagcaagccaaaagtcaccggaacatcaagc
tgctggaggacgagcccaggagccgagacgagactcctctgtgtaccatcgcgcactgg
caggactcgctggctaagcgatgcatctgtgtgtccaatattgtccgtagcttgtcatt
cgtgcctggcaatgatgccgaaatgtccaaacatccaggcctggtgctgatcctgggga
agctgattcttcttcaccacgagcatccagagagaaagcgagcaccgcagacctatgag
aaagaggaggatgaggacaagggggtggcctgcagcaaagatgagtggtggtgggactg
cctcgaggtcttgagggataacacgttggtcacgttggccaacatttccgggcagctag
acttgtctgcttacacggaaagcatctgcttgccaattttggatggcttgctgcactgg
atggtgtgcccgtctgcagaggcacaagatccctttccaactgtgggacccaactcggt
cctgtcgcctcagagacttgtgctggagaccctctgtaaactcagtatccaggacaata
atgtggacctgatcttggccactcctccatttagtcgtcaggagaaattctatgctaca
ttagttaggtacgttggggatcgcaaaaacccagtctgtcgagaaatgtccatggcgct
tttatcgaaccttgcccaaggggacgcactagcagcaagggccatagctgtgcagaaag
gaagcattggaaacttgataagcttcctagaggatggggtcacgatggcccagtaccag
cagagccagcacaacctcatgcacatgcagcccccgcccctggaaccacctagcgtaga
catgatgtgcagggcggccaaggctttgctagccatggccagagtggacgaaaaccgct
cggaattccttttgcacgagggccggttgctggatatctcgatatcagctgtcctgaac
tctctggttgcatctgtcatctgtgatgtactgtttcagattgggcagttatgacataa
gtgagaaggcaagcatgtgtgagtgaagattagagggtcacatataactggctgttttc
tgttcttgtttatccagcgtaggaagaaggaaaagaaaatctttgctcctctgccccat
tcactatttaccaattgggaattaaagaaataattaatttgaacagttatgaaattaat
atttgctgtctgtgtgtataagtacatcctttggggttttttttttctcttttttttaa
ccaaagttgctgtctagtgcattcaaaggtcactttttgttcttcacagatctttttaa
tgttctttcccatgttgtattgcatttttgggggaagcaaattgactttaaagaaaaaa
gttgtggcaaaagatgctaagatgcgaaaatttcaccacactgagtcaaaaaggtgaaa
aattatccatttcctatgcgttttactcctcagagaatgaaaaaaactgcatcccatca
cccaaagttctgtgcaatagaaatttctacagatacaggtataggggctcaaggaggta
tgtcggtcagtagtcaaaactatgaaatgatactggtttctccacaggaatatggttcc
attaggctgggagcaaaaacaatgttttttaagattgagaatacatacctgacaacgat
ccggaaactgctcctcaccactcccgtcatgcctgctgtcggcgtttgaccttccacgt
gacagttcttcacaattcctttcatcattttttaaatattttttttactgcctatgggc
tgtgatgtatatagaagttgtacattaaacataccctcatttttttcttttcttttttt
tttttttttttagtacaaagttttagtttctttttcatgatgtggtaactacgaagtga
tggtagatttaaataattttttatttttattttatatattttttcattagggccatatc
tccaaaaaaagaaagaaaaaatacaaaaaacaaaaacaaaaaaaaaagagggtaatgta
caagtttctgtatgtataaagtcatgctcgatttcaggagagcagctgatcacaatttg
cttcatgaatcaaggtgtggaaatggttatatatggattgatttagaaaatggttacca
gtacagtcaaaaaagagaaaatgaaaaaaatacaactaaaaggaagaaacacaacttca
aagatttttcagtgatgagaatccacatttgtatttcaagataatgtagtttaaaaaaa
aaaaaaagaaaaaaacttgatgtaaattcctccttttcctctggcttaatgaatatcat
ttattcagtataaaatctttatatgttccacatgttaagaataaatgtacattaaatct
tgttaagcactgtgatgggtgttcttgaatactgttctagtttccttaaagtggtttcc
tagtaatcaagttatttacaagaaataggggaatgcagcagtgtattcacattataaaa
ccctacatttggaagagacctttaggggttacctactttagagtggggagcaacagttt
gattttctcaaattacttagctaattagtctttctttgaagcaattaactctaacgaca
ttgaggtatgatcattttcagtatttatgggaggtggctgctgacccacttgaggtgag
atctcagaagcttaactggcctgaaaatgtaacattctgccttttactaactccatctt
agtttaatcaaagttcaatctattccttgtttcttctgtgtgcctcagagttattttgc
atttagtttactccaccgtgtataatatttatactgtgcaatgttaaaaaagaatctgt
tatattgtatgtggtgtacatagtgcaaagtgatgatttctatttcagggcatattatg
gttctcatattccttcctacctggtgcacagtagctttttaatactagtcacttctaat
ttaaactttctcttcctgggtcattgactgttactgtgtaataatcgatttctttgaaa
ctgctgcataattatgctgttagtggacctctacctcttctcttccctctcccaatcac
agtatactcagaatccccagcccctcgcatacattgtgtcggttcacattactcacagt
aatatatggaagagttagacaagaacatgcagttacagtcattgtgagacgtgactctc
cagtgtcacgaggaaaaaaatcatcttttctgcaaacagtctctcatctgtcaactccc
acattactgagtcaaacagtcttcttacataacaatgcaaccaaatatatgttgaatta
aagacccatttataattctgctttaaatacatctgcttgctaagaacagatttcagtgc
tccaagcttcaaatatggagatttgtaagagggaattcaatattattctaatttctctc
ttacagagtacaaataaaaggtgtatacaaactccgaacatatccagtattccaattcc
tttgtcaatcagaagagtaaaataattaacaaaagactgttgttatggtttgcattgta
accgatacgcagagtctgaccgttgggcaacaagtttttctatcctgatgcgcaacaca
gtctctagagactaatccaggaagactttagcctcctttccatattctcacccccgaat
caagatttacagaagcccacgaagaatttacagcctgcttgagatcatcttgcctataa
actgagttattgctttgtcctaaaaattagtcggtttttttttttctatgaggcttttc
agaaatttacaggatgcccagactttacatgtgtaccaaaaaaaaaaaaaagataaaaa
ataaaggtgcaaagaaagtttagtattttggaatggtgctataaagttgaa
SEQ ID NO: 21
MMMMALSKTFGQKPVKFQLEDDGEFYMIGSEVGNYLRMFRGSLYKRYPSLWRRLATVEE
RKKIVASSHGKKTKPNTKDHGYTTLATSVTLLKASEVEEILDGNDEKYKAVSISTEPPT
YLREQKAKRNSQWVPTLPNSSHHLDAVPCSTTINRNRMGRDKKRTFPLCFDDHDPAVIH
ENASQPEVLVPIRLDMEIDGQKLRDAFTWNMNEKLMTPEMFSEILCDDLDLNPLTFVPA
IASAIRQQIESYPTDSILEDQSDQRVIIKLNIHVGNISLVDQFEWDMSEKENSPEKFAL
KLCSELGLGGEFVTTIAYSIRGQLSWHQKTYAFSENPLPTVEIAIRNTGDADQWCPLLE
TLTDAEMEKKIRDQDRNTRRMRRLANTAPAW
SEQ ID NO: 22
MMMMALSKTFGQKPVKFQLEDDGEFYMIGSEVGNYLRMFRGSLYKRYPSLWRRLATVEE
RKKIVASSHDHGYTTLATSVTLLKASEVEEILDGNDEKYKAVSISTEPPTYLREQKAKR
NSQWVPTLPNSSHHLDAVPCSTTINRNRMGRDKKRTFPLCFDDHDPAVIHENASQPEVL
VPIRLDMEIDGQKLRDAFTWNMNEKLMTPEMFSEILCDDLDLNPLTFVPAIASAIRQQI
ESYPTDSILEDQSDQRVIIKLNIHVGNISLVDQFEWDMSEKENSPEKFALKLCSELGLG
GEFVTTIAYSIRGQLSWHQKTYAFSENPLPTVEIAIRNTGDADQWCPLLETLTDAEMEK
KIRDQDRNTRRMRRLANTAPAW
SEQ ID NO: 23
MSTPTDPGAMPHPGPSPGPGPSPGPILGPSPGPGPSPGSVHSMMGPSPGPPSVSHPMPT
MGSTDFPQEGMHQMHKPIDGIHDKGIVEDIHCGSMKGTGMRPPHPGMGPPQSPMDQHSQ
GYMSPHPSPLGAPEHVSSPMSGGGPTPPQMPPSQPGALIPGDPQAMSQPNRGPSPFSPV
QLHQLRAQILAYKMLARGQPLPETLQLAVQGKRTLPGLQQQQQQQQQQQQQQQQQQQQQ
QQPQQQPPQPQTQQQQQPALVNYNRPSGPGPELSGPSTPQKLPVPAPGGRPSPAPPAAA
QPPAAAVPGPSVPQPAPGQPSPVLQLQQKQSRISPIQKPQGLDPVEILQEREYRL
QARIAHRIQELENLPGSLPPDLRTKATVELKALRLLNFQRQLRQEVVACMRRDTTLETA
LNSKAYKRSKRQTLREARMTEKLEKQQKIEQERKRRQKHQEYLNSILQHAKDFKEYHRS
VAGKIQKLSKAVATWHANTEREQKKETERIEKERMRRLMAEDEEGYRKLIDQKKDRRLA
YLLQQTDEYVANLINLVWEHKQAQAAKEKKKRRRRKKKAEENAEGGESALGPDGEPIDE
SSQMSDLPVKVTHTETGKVLFGPEAPKASQLDAWLEMNPGYEVAPRSDSEESDSDYEEE
DEEEESSRQETEEKILLDPNSEEVSEKDAKQIIETAKQDVDDEYSMQYSARGSQS
YYTVAHAISERVEKQSALLINGTLKHYQLQGLEWMVSLYNNNLNGILADEMGLGKTIQT
IALITYLMEHKRLNGPYLIIVPLSTLSNWTYEFDKWAPSVVKISYKGTPAMRRSLVPQL
RSGKFNVLLTTYEYIIKDKHILAKIRWKYMIVDEGHRMKNHHCKLTQVLNTHYVAPRRI
LLTGTPLQNKLPELWALLNFLLPTIFKSCSTFEQWENAPFAMTGERVDLNEEETILIIR
RLHKVLRPFLLRRLKKEVESQLPEKVEYVIKCDMSALQKILYRHMQAKGILLTDGSEKD
KKGKGGAKTLMNTIMQLRKICNHPYMFQHIEESFAEHLGYSNGVINGAELYRASG
KFELLDRILPKLRATNHRVLLFCQMTSLMTIMEDYFAFRNFLYLRLDGTTKSEDRAALL
KKENEPGSQYFIFLLSTRAGGLGLNLQAADTVVIFDSDWNPHQDLQAQDRAHRIGQQNE
VRVLRLCTVNSVEEKILAAAKYKLNVDQKVIQAGMFDQKSSSHERRAFLQAILEHEEEN
EEEDEVPDDETLNQMIARREEEFDLFMRMDMDRRREDARNPKRKPRLMEEDELPSWIIK
DDAEVERLTCEEEEEKIFGRGSRQRRDVDYSDALTEKQWLRAIEDGNLEEMEEEVRLKK
RKRRRNVDKDPAKEDVEKAKKRRGRPPAEKLSPNPPKLTKQMNAIIDTVINYKDR
CNVEKVPSNSQLEIEGNSSGRQLSEVFIQLPSRKELPEYYELIRKPVDFKKIKERIRNH
KYRSLGDLEKDVMLLCHNAQTFNLEGSQIYEDSIVLQSVFKSARQKIAKEEESEDESNE
EEEEEDEEESESEAKSVKVKIKLNKKDDKGRDKGKGKKRPNRGKAKPVVSDFDSDEEQD
EREQSEGSGTDDE
SEQ ID NO: 24
MSTPTDPGAMPHPGPSPGPGPSPGPILGPSPGPGPSPGSVHSMMGPSPGPPSVSHPMPT
MGSTDFPQEGMHQMHKPIDGIHDKGIVEDIHCGSMKGTGMRPPHPGMGPPQSPMDQHSQ
GYMSPHPSPLGAPEHVSSPMSGGGPTPPQMPPSQPGALIPGDPQAMSQPNRGPSPFSPV
QLHQLRAQILAYKMLARGQPLPETLQLAVQGKRTLPGLQQQQQQQQQQQQQQQQQQQQQ
QQPQQQPPQPQTQQQQQPALVNYNRPSGPGPELSGPSTPQKLPVPAPGGRPSPAPPAAA
QPPAAAVPGPSVPQPAPGQPSPVLQLQQKQSRISPIQKPQGLDPVEILQEREYRL
QARIAHRIQELENLPGSLPPDLRTKATVELKALRLLNFQRQLRQEVVACMRRDTTLETA
LNSKAYKRSKRQTLREARMTEKLEKQQKIEQERKRRQKHQEYLNSILQHAKDFKEYHRS
VAGKIQKLSKAVATWHANTEREQKKETERIEKERMRRLMAEDEEGYRKLIDQKKDRRLA
YLLQQTDEYVANLTNLVWEHKQAQAAKEKKKRRRRKKKAEENAEGGESALGPDGEPIDE
SSQMSDLPVKVTHTETGKVLFGPEAPKASQLDAWLEMNPGYEVAPRSDSEESDSDYEEE
DEEEESSRQETEEKILLDPNSEEVSEKDAKQIIETAKQDVDDEYSMQYSARGSQS
YYTVAHAISERVEKQSALLINGTLKHYQLQGLEWMVSLYNNNLNGILADEMGLGKTIQT
IALITYLMEHKRLNGPYLIIVPLSTLSNWTYEFDKWAPSVVKISYKGTPAMRRSLVPQL
RSGKFNVLLTTYEYIIKDKHILAKIRWKYMIVDEGHRMKNHHCKLTQVLNTHYVAPRRI
LLTGTPLQNKLPELWALLNFLLPTIFKSCSTFEQWFNAPFAMTGERVDLNEEETILIIR
RLHKVLRPFLLRRLKKEVESQLPEKVEYVIKCDMSALQKILYRHMQAKGILLTDGSEKD
KKGKGGAKTLMNTIMQLRKICNHPYMFQHIEESFAEHLGYSNGVINGAELYRASG
KFELLDRILPKLRATNHRVLLFCQMTSLMTIMEDYFAFRNFLYLRLDGTTKSEDRAALL
KKFNEPGSQYFIFLLSTRAGGLGLNLQAADTVVIFDSDWNPHQDLQAQDRAHRIGQQNE
VRVLRLCTVNSVEEKILAAAKYKLNVDQKVIQAGMFDQKSSSHERRAFLQAILEHEEEN
EEEDEVPDDETLNQMIARREEEFDLEMRMDMDRRREDARNPKRKPRLMEEDELPSWIIK
DDAEVERLTCEEEEEKIFGRGSRQRRDVDYSDALTEKQWLRAIEDGNLEEMEEEVRLKK
RKRRRNVDKDPAKEDVEKAKKRRGRPPAEKLSPNPPKLTKQMNAIIDTVINYKDS
SGRQLSEVFIQLPSRKELPEYYELIRKPVDFKKIKERIRNHKYRSLGDLEKDVMLLCHN
AQTENLEGSQIYEDSIVLQSVFKSARQKIAKEEESEDESNEEEEEEDEEESESEAKSVK
VKIKLNKKDDKGRDKGKGKKRPNRGKAKPVVSDFDSDEEQDEREQSEGSGTDDE
SEQ ID NO: 25
MSTPDPPLGGTPRPGPSPGPGPSPGAMLGPSPGPSPGSAHSMMGPSPGPPSAGHPIPTQ
GPGGYPQDNMHQMHKPMESMHEKGMSDDPRYNQMKGMGMRSGGHAGMGPPPSPMDQHSQ
GYPSPLGGSEHASSPVPASGPSSGPQMSSGPGGAPLDGADPQALGQQNRGPTPFNQNQL
HQLRAQIMAYKMLARGQPLPDHLQMAVQGKRPMPGMQQQMPTLPPPSVSATGPGPGPGP
GPGPGPGPAPPNYSRPHGMGGPNMPPPGPSGVPPGMPGQPPGGPPKPWPEGPMANAAAP
TSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRL
LNFQRQLRQEVVVCMRRDTALETALNAKAYKRSKRQSLREARITEKLEKQQKIEQERKR
RQKHQEYLNSILQHAKDFKEYHRSVTGKIQKLTKAVATYHANTEREQKKENERIEKERM
RRLMAEDEEGYRKLIDQKKDKRLAYLLQQTDEYVANLTELVRQHKAAQVAKEKKKKKKK
KKAENAEGQTPAIGPDGEPLDETSQMSDLPVKVIHVESGKILTGTDAPKAGQLEAWLEM
NPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQI
KGLEWLVSLYNNNLNGILADEMGLGKTIQTIALITYLMEHKRINGPFLIIVPLSTLSNW
AYEFDKWAPSVVKVSYKGSPAARRAFVPQLRSGKFNVLLTTYEYIIKDKHILAKIRWKY
MIVDEGHRMKNHHCKLTQVLNTHYVAPRRLLLIGTPLQNKLPELWALLNFLLPTIFKSC
STFEQWFNAPFAMTGEKVDLNEEETILIIRRLHKVLRPFLLRRLKKEVEAQLPEKVEYV
IKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLM
TIMEDYFAYRGFKYLRLDGTTKAEDRGMLLKTFNEPGSEYFIFLLSTRAGGLGLNLQSA
DTVIIFDSDWNPHQDLQAQDRAHRIGQQNEVRVLRLCTVNSVEEKILAAAKYKLNVDQK
VIQAGMFDQKSSSHERRAFLQAILEHEEQDESRHCSTGSGSASFAHTAPPPAGVNPDLE
EPPLKEEDEVPDDETVNQMIARHEEEFDLEMRMDLDRRREEARNPKRKPRLMEEDELPS
WIIKDDAEVERLTCEEEEEKMFGRGSRHRKEVDYSDSLTEKQWLKAIEEGTLEEI
EEEVRQKKSSRKRKRDSDAGSSTPTTSTRSRDKDDESKKQKKRGRPPAEKLSPNPPNLT
KKMKKIVDAVIKYKDSSSGRQLSEVFIQLPSRKELPEYYELIRKPVDFKKIKERIRNHK
YRSLNDLEKDVMLLCQNAQTFNLEGSLIYEDSIVLQSVFTSVRQKIEKEDDSEGEESEE
EEEGEEEGSESESRSVKVKIKLGRKEKAQDRLKGGRRRPSRGSRAKPVVSDDDSEEEQE
EDRSGSGSEED
SEQ ID NO: 26
MSTPDPPLGGTPRPGPSPGPGPSPGAMLGPSPGPSPGSAHSMMGPSPGPPSAGHPIPTQ
GPGGYPQDNMHQMHKPMESMHEKGMSDDPRYNQMKGMGMRSGGHAGMGPPPSPMDQHSQ
GYPSPLGGSEHASSPVPASGPSSGPQMSSGPGGAPLDGADPQALGQQNRGPTPFNQNQL
HQLRAQIMAYKMLARGQPLPDHLQMAVQGKRPMPGMQQQMPTLPPPSVSATGPGPGPGP
GPGPGPGPAPPNYSRPHGMGGPNMPPPGPSGVPPGMPGQPPGGPPKPWPEGPMANAAAP
TSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRL
LNFQRQLRQEVVVCMRRDTALETALNAKAYKRSKRQSLREARITEKLEKQQKIEQERKR
RQKHQEYLNSILQHAKDFKEYHRSVTGKIQKLTKAVATYHANTEREQKKENERIEKERM
RRLMAEDEEGYRKLIDQKKDKRLAYLLQQTDEYVANLTELVRQHKAAQVAKEKKKKKKK
KKAENAEGQTPAIGPDGEPLDETSQMSDLPVKVIHVESGKILTGTDAPKAGQLEAWLEM
NPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQI
KGLEWLVSLYNNNLNGILADEMGLGKTIQTIALITYLMEHKRINGPFLIIVPLSTLSNW
AYEFDKWAPSVVKVSYKGSPAARRAFVPQLRSGKENVLLTTYEYIIKDKHILAKIRWKY
MIVDEGHRMKNHHCKLTQVLNTHYVAPRRLLLIGTPLQNKLPELWALLNFLLPTIFKSC
STFEQWENAPFAMTGEKVDLNEEETILIIRRLHKVLRPFLLRRLKKEVEAQLPEKVEYV
IKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLM
TIMEDYFAYRGFKYLRLDGTTKAEDRGMLLKTFNEPGSEYFIFLLSTRAGGLGLNLQSA
DTVIIFDSDWNPHQDLQAQDRAHRIGQQNEVRVLRLCTVNSVEEKILAAAKYKLNVDQK
VIQAGMFDQKSSSHERRAFLQAILEHEEQDEEEDEVPDDETVNQMIARHEEEFDLFMRM
DLDRRREEARNPKRKPRLMEEDELPSWIIKDDAEVERLTCEEEEEKMFGRGSRHRKEVD
YSDSLTEKQWLKAIEEGTLEEIEEEVRQKKSSRKRKRDSDAGSSTPTTSTRSRDK
DDESKKQKKRGRPPAEKLSPNPPNLTKKMKKIVDAVIKYKDSSSGRQLSEVFIQLPSRK
ELPEYYELIRKPVDFKKIKERIRNHKYRSLNDLEKDVMLLCQNAQTFNLEGSLIYEDSI
VLQSVFTSVRQKIEKEDDSEGEESEEEEEGEEEGSESESRSVKVKIKLGRKEKAQDRLK
GGRRRPSRGSRAKPVVSDDDSEEEQEEDRSGSGSEED
ARID1A-1
SEQ ID NO: 27
MAAQVAPAAASSLGNPPPPPPSELKKAEQQQREEAGGEAAAAAAAERGEMKAAAGQESE
GPAVGPPQPLGKELQDGAESNGGGGGGGAGSGGGPGAEPDLKNSNGNAGPRPALNNNLT
EPPGGGGGGSSDGVGAPPHSAAAALPPPAYGFGQPYGRSPSAVAAAAAAVFHQQHGGQQ
SPGLAALQSGGGGGLEPYAGPQQNSHDHGFPNHQYNSYYPNRSAYPPPAPAYALSSPRG
GTPGSGAAAAAGSKPPPSSSASASSSSSSFAQQRFGAMGGGGPSAAGGGTPQPTATPTL
NQLLTSPSSARGYQGYPGGDYSGGPQDGGAGKGPADMASQCWGAAAAAAAAAAASGGAQ
QRSHHAPMSPGSSGGGGQPLARTPQPSSPMDQMGKMRPQPYGGTNPYSQQQGPPSGPQQ
GHGYPGQPYGSQTPQRYPMTMQGRAQSAMGGLSYTQQIPPYGQQGPSGYGQQGQTPYYN
QQSPHPQQQQPPYSQQPPSQTPHAQPSYQQQPQSQPPQLQSSQPPYSQQPSQPPHQQSP
APYPSQQSTTQQHPQSQPPYSQPQAQSPYQQQQPQQPAPSTLSQQAAYPQPQSQQSQQT
AYSQQRFPPPQELSQDSFGSQASSAPSMTSSKGGQEDMNLSLQSRPSSLPDLSGSIDDL
PMGTEGALSPGVSTSGISSSQGEQSNPAQSPFSPHTSPHLPGIRGPSPSPVGSPASVAQ
SRSGPLSPAAVPGNQMPPRPPSGQSDSIMHPSMNQSSIAQDRGYMQRNPQMPQYSSPQP
GSALSPRQPSGGQIHTGMGSYQQNSMGSYGPQGGQYGPQGGYPRQPNYNALPNANYPSA
GMAGGINPMGAGGQMHGQPGIPPYGTLPPGRMSHASMGNRPYGPNMANMPPQVGSGMCP
PPGGMNRKTQETAVAMHVAANSIQNRPPGYPNMNQGGMMGTGPPYGQGINSMAGMINPQ
GPPYSMGGTMANNSAGMAASPEMMGLGDVKLTPATKMNNKADGTPKTESKSKKSSSSTT
TNEKITKLYELGGEPERKMWVDRYLAFTEEKAMGMTNLPAVGRKPLDLYRLYVSVKEIG
GLTQVNKNKKWRELATNLNVGTSSSAASSLKKQYIQCLYAFECKIERGEDPPPDIFAAA
DSKKSQPKIQPPSPAGSGSMQGPQTPQSTSSSMAEGGDLKPPTPASTPHSQIPPLPGMS
RSNSVGIQDAFNDGSDSTFQKRNSMTPNPGYQPSMNTSDMMGRMSYEPNKDPYGSMRKA
PGSDPFMSSGQGPNGGMGDPYSRAAGPGLGNVAMGPRQHYPYGGPYDRVRTEPGIGPEG
NMSTGAPQPNLMPSNPDSGMYSPSRYPPQQQQQQQQRHDSYGNQFSTQGTPSGSPFPSQ
QTTMYQQQQQNYKRPMDGTYGPPAKRHEGEMYSVPYSTGQGQPQQQQLPPAQPQPASQQ
QAAQPSPQQDVYNQYGNAYPATATAATERRPAGGPQNQFPFQFGRDRVSAPPGTNAQQN
MPPQMMGGPIQASAEVAQQGTMWQG
RNDMTYNYANRQSTGSAPQGPAYHGVNRTDEMLHTDQRANHEGSWPSHGTRQPPYGPSA
PVPPMTRPPPSNYQPPPSMQNHIPQVSSPAPLPRPMENRTSPSKSPFLHSGMKMQKAGP
PVPASHIAPAPVQPPMIRRDITFPPGSVEATQPVLKQRRRLTMKDIGTPEAWRVMMSLK
SGLLAESTWALDTINILLYDDNSIMTFNLSQLPGLLELLVEYFRRCLIEIFGILKEYEV
GDPGQRTLLDPGRFSKVSSPAPMEGGEEEEELLGPKLEEEEEEEVVENDEEIAFSGKDK
PASENSEEKLISKFDKLPVKIVQKNDPFVVDCSDKLGRVQEFDSGLLHWRIGGGDTTEH
IQTHFESKTELLPSRPHAPCPPAPRKHVTTAEGTPGTTDQEGPPPDGPPEKRITATMDD
MLSTRSSTLTEDGAKSSEAIKESSKFPFGISPAQSHRNIKILEDEPHSKDETPLCTLLD
WQDSLAKRCVCVSNTIRSLSFVPGNDFEMSKHPGLLLILGKLILLHHKHPERKQAPLTY
EKEEEQDQGVSCNKVEWWWDCLEMLRENTLVTLANISGQLDLSPYPESICLPVLDGLLH
WAVCPSAEAQDPFSTLGPNAVLSPQRLVLETLSKLSIQDNNVDLILATPPFSRLEKLYS
TMVRFLSDRKNPVCREMAVVLLANLAQGDSLAARAIAVQKGSIGNLLGFLEDSLAATQF
QQSQASLLHMQNPPFEPTSVDMMRRAARALLALAKVDENHSEFTLYESRLLDISVSPLM
NSLVSQVICDVLFLIGQS
ARID1A-2
SEQ ID NO: 28
MAAQVAPAAASSIGNPPPPPPSELKKAEQQQREEAGGEAAAAAAAERGEMKAAAGQESE
GPAVGPPQPLGKELQDGAESNGGGGGGGAGSGGGPGAEPDLKNSNGNAGPRPALNNNLT
EPPGGGGGGSSDGVGAPPHSAAAALPPPAYGFGQPYGRSPSAVAAAAAAVFHQQHGGQQ
SPGLAALQSGGGGGLEPYAGPQQNSHDHGFPNHQYNSYYPNRSAYPPPAPAYALSSPRG
GTPGSGAAAAAGSKPPPSSSASASSSSSSFAQQRFGAMGGGGPSAAGGGTPQPTATPTL
NQLLTSPSSARGYQGYPGGDYSGGPQDGGAGKGPADMASQCWGAAAAAAAAAAASGGAQ
QRSHHAPMSPGSSGGGGQPLARTPQPSSPMDQMGKMRPQPYGGINPYSQQQGPPSGPQQ
GHGYPGQPYGSQTPQRYPMTMQGRAQSAMGGLSYTQQIPPYGQQGPSGYGQQGQTPYYN
QQSPHPQQQQPPYSQQPPSQTPHAQPSYQQQPQSQPPQLQSSQPPYSQQPSQPPHQQSP
APYPSQQSTTQQHPQSQPPYSQPQAQSPYQQQQPQQPAPSTLSQQAAYPQPQSQQSQQT
AYSQQRFPPPQELSQDSFGSQASSAPSMTSSKGGQEDMNLSLQSRPSSLPDLSGSIDDL
PMGTEGALSPGVSTSGISSSQGEQSNPAQSPFSPHTSPHLPGIRGPSPSPVGSPASVAQ
SRSGPLSPAAVPGNQMPPRPPSGQSDSIMHPSMNQSSIAQDRGYMQRNPQMPQYSSPQP
GSALSPRQPSGGQIHTGMGSYQQNSMGSYGPQGGQYGPQGGYPRQPNYNALPNANYPSA
GMAGGINPMGAGGQMHGQPGIPPYGTLPPGRMSHASMGNRPYGPNMANMPPQVGSGMCP
PPGGMNRKTQETAVAMHVAANSIQNRPPGYPNMNQGGMMGTGPPYGQGINSMAGMINPQ
GPPYSMGGTMANNSAGMAASPEMMGLGDVKLTPATKMNNKADGTPKTESKSKKSSSSTT
TNEKITKLYELGGEPERKMWVDRYLAFTEEKAMGMTNLPAVGRKPLDLYRLYVSVKEIG
GLTQVNKNKKWRELATNLNVGTSSSAASSLKKQYIQCLYAFECKIERGEDPPPDIFAAA
DSKKSQPKIQPPSPAGSGSMQGPQTPQSTSSSMAEGGDLKPPTPASTPHSQIPPLPGMS
RSNSVGIQDAFNDGSDSTFQKRNSMTPNPGYQPSMNTSDMMGRMSYEPNKDPYGSMRKA
PGSDPFMSSGQGPNGGMGDPYSRAAGPGLGNVAMGPRQHYPYGGPYDRVRTEPGIGPEG
NMSTGAPQPNLMPSNPDSGMYSPSRYPPQQQQQQQQRHDSYGNQFSTQGTPSGSPFPSQ
QTTMYQQQQQVSSPAPLPRPMENRTSPSKSPFLHSGMKMQKAGPPVPASHIAPAPVQPP
MIRRDITFPPGSVEATQPVLKQRRRLTMKDIGTPEAWRVMMSLKSGLLAESTWALDTIN
ILLYDDNSIMTFNLSQLPGLLELLVEYFRRCLIEIFGILKEYEVGDPGQRTLLDPGRFS
KVSSPAPMEGGEEEEELLGPKLEEEEEEEVVENDEEIAFSGKDKPASENSEEKLISKFD
KLPVKIVQKNDPFVVDCSDKLGRVQEFDSGLLHWRIGGGDTTEHIQTHFESKTELLPSR
PHAPCPPAPRKHVTTAEGTPGTTDQEGPPPDGPPEKRITATMDDMLSTRSSTLTEDGAK
SSEAIKESSKFPFGISPAQSHRNIKILEDEPHSKDETPLCTLLDWQDSLAKRCVCVSNT
IRSLSFVPGNDFEMSKHPGLLLILGKLILLHHKHPERKQAPLTYEKEEEQDQGVSCNKV
EWWWDCLEMLRENTLVTLANISGQLDLSPYPESICLPVLDGLLHWAVCPSAEAQDPFST
LGPNAVLSPQRLVLETLSKLSIQDNNVDLILATPPFSRLEKLYSTMVRFLSDRKNPVCR
EMAVVLLANLAQGDSLAARAIAVQKGSIGNLLGFLEDSLAATQFQQSQASLLHMQNPPF
EPTSVDMMRRAARALLALAKVDENHSEFTLYESRLLDISVSPLMNSLVSQVICDVLFLI
GQS
ARID1A-3
SEQ ID NO: 29
MDQMGKMRPQPYGGTNPYSQQQGPPSGPQQGHGYPGQPYGSQTPQRYPMTMQGRAQSAM
GGLSYTQQIPPYGQQGPSGYGQQGQTPYYNQQSPHPQQQQPPYSQQPPSQTPHAQPSYQ
QQPQSQPPQLQSSQPPYSQQPSQPPHQQSPAPYPSQQSTTQQHPQSQPPYSQPQAQSPY
QQQQPQQPAPSTLSQQAAYPQPQSQQSQQTAYSQQRFPPPQELSQDSFGSQASSAPSMT
SSKGGQEDMNLSLQSRPSSLPDLSGSIDDLPMGTEGALSPGVSTSGISSSQGEQSNPAQ
SPFSPHTSPHLPGIRGPSPSPVGSPASVAQSRSGPLSPAAVPGNQMPPRPPSGQSDSIM
HPSMNQSSIAQDRGYMQRNPQMPQYSSPQPGSALSPRQPSGGQIHTGMGSYQQNSMGSY
GPQGGQYGPQGGYPRQPNYNALPNANYPSAGMAGGINPMGAGGQMHGQPGIPPYGTLPP
GRMSHASMGNRPYGPNMANMPPQVGSGMCPPPGGMNRKTQETAVAMHVAANSIQNRPPG
YPNMNQGGMMGTGPPYGQGINSMAGMINPQGPPYSMGGTMANNSAGMAASPEMMGLGDV
KLTPATKMNNKADGTPKTESKSKKSSSSTTTNEKITKLYELGGEPERKMWVDRYLAFTE
EKAMGMTNLPAVGRKPLDLYRLYVSVKEIGGLTQVNKNKKWRELATNLNVGTSSSAASS
LKKQYIQCLYAFECKIERGEDPPPDIFAAADSKKSQPKIQPPSPAGSGSMQGPQTPQST
SSSMAEGGDLKPPTPASTPHSQIPPLPGMSRSNSVGIQDAFNDGSDSTFQKRNSMTPNP
GYQPSMNTSDMMGRMSYEPNKDPYGSMRKAPGSDPFMSSGQGPNGGMGDPYSRAAGPGL
GNVAMGPRQHYPYGGPYDRVRTEPGIGPEGNMSTGAPQPNLMPSNPDSGMYSPSRYPPQ
QQQQQQQRHDSYGNQFSTQGTPSGSPFPSQQTTMYQQQQQNYKRPMDGTYGPPAKRHEG
EMYSVPYSTGQGQPQQQQLPPAQPQPASQQQAAQPSPQQDVYNQYGNAYPATATAATER
RPAGGPQNQFPFQFGRDRVSAPPGTNAQQNMPPQMMGGPIQASAEVAQQGTMWQGRNDM
TYNYANRQSTGSAPQGPAYHGVNRTDEMLHTDQRANHEGSWPSHGTRQPPYGPSAPVPP
MTRPPPSNYQPPPSMQNHIPQVSSPAPLPRPMENRTSPSKSPFLHSGMKMQKAGPPVPA
SHIAPAPVQPPMIRRDITFPPGSVEATQPVLKQRRRLTMKDIGTPEAWRVMMSLKSGLL
AESTWALDTINILLYDDNSIMTFNLSQLPGLLELLVEYFRRCLIEIFGILKEYEVGDPG
QRTLLDPGRFSKVSSPAPMEGGEEEEELLGPKLEEEEEEEVVENDEEIAFSGKDKPASE
NSEEKLISKFDKLPVKIVQKNDPFVVDCSDKLGRVQEFDSGLLHWRIGGGDTTEHIQTH
FESKTELLPSRPHAPCPPAPRKHVTTAEGTPGTTDQEGPPPDGPPEKRITATMDDMLST
RSSTLTEDGAKSSEAIKESSKFPFGISPAQSHRNIKILEDEPHSKDETPLCTLLDWQDS
LAKRCVCVSNTIRSLSFVPGNDFEMSKHPGLLLILGKLILLHHKHPERKQAPLTYEKEE
EQDQGVSCNKVEWWWDCLEMLRENTLVTLANISGQLDLSPYPESICLPVLDGLLHWAVC
PSAEAQDPFSTLGPNAVLSPQRLVLETLSKLSIQDNNVDLILATPPFSRLEKLYSTMVR
FLSDRKNPVCREMAVVLLANLAQGDSLAARAIAVQKGSIGNLLGFLEDSLAATQFQQSQ
ASLLHMQNPPFEPTSVDMMRRAARALLALAKVDENHSEFTLYESRLLDISVSPLMNSLV
SQVICDVLFLIGQS
ARID1B-1
SEQ ID NO: 30
MAHNAGAAAAAGTHSAKSGGSEAALKEGGSAAALSSSSSSSAAAAAASSSSSSGPGSAM
ETGLLPNHKLKTVGEAPAAPPHQQHHHHHHAHHHHHHAHHLHHHHALQQQLNQFQQQQQ
QQQQQQQQQQQQQHPISNNNSLGGAGGGAPQPGPDMEQPQHGGAKDSAAGGQADPPGPP
LLSKPGDEDDAPPKMGEPAGGRYEHPGLGALGTQQPPVAVPGGGGGPAAVPEFNNYYGS
AAPASGGPGGRAGPCFDQHGGQQSPGMGMMHSASAAAAGAPGSMDPLQNSHEGYPNSQC
NHYPGYSRPGAGGGGGGGGGGGGGSGGGGGGGGAGAGGAGAGAVAAAAAAAAAAAGGGG
GGGYGGSSAGYGVLSSPRQQGGGMMMGPGGGGAASLSKAAAGSAAGGFQRFAGQNQHPS
GATPTLNQLLTSPSPMMRSYGGSYPEYSSPSAPPPPPSQPQSQAAAAGAAAGGQQAAAG
MGLGKDMGAQYAAASPAWAAAQQRSHPAMSPGTPGPTMGRSQGSPMDPMVMKRPQLYGM
GSNPHSQPQQSSPYPGGSYGPPGPQRYPIGIQGRTPGAMAGMQYPQQQMPPQYGQQGVS
GYCQQGQQPYYSQQPQPPHLPPQAQYLPSQSQQRYQPQQDMSQEGYGTRSQPPLAPGKP
NHEDLNLIQQERPSSLPDLSGSIDDLPTGTEATLSSAVSASGSTSSQGDQSNPAQSPFS
PHASPHLSSIPGGPSPSPVGSPVGSNQSRSGPISPASIPGSQMPPQPPGSQSESSSHPA
LSQSPMPQERGFMAGTQRNPQMAQYGPQQTGPSMSPHPSPGGQMHAGISSFQQSNSSGT
YGPQMSQYGPQGNYSRPPAYSGVPSASYSGPGPGMGISANNQMHGQGPSQPCGAVPLGR
MPSAGMQNRPFPGNMSSMTPSSPGMSQQGGPGMGPPMPTVNRKAQEAAAAVMQAAANSA
QSRQGSFPGMNQSGLMASSSPYSQPMNNSSSLMNTQAPPYSMAPAMVNSSAASVGLADM
MSPGESKLPLPLKADGKEEGTPQPESKSKKSSSSTTTGEKITKVYELGNEPERKLWVDR
YLTFMEERGSPVSSLPAVGKKPLDLFRLYVCVKEIGGLAQVNKNKKWRELATNLNVGTS
SSAASSLKKQYIQYLFAFECKIERGEEPPPEVFSTGDTKKQPKLQPPSPANSGSLQGPQ
TPQSTGSNSMAEVPGDLKPPTPASTPHGQMTPMQGGRSSTISVHDPFSDVSDSSFPKRN
SMTPNAPYQQGMSMPDVMGRMPYEPNKDPFGGMRKVPGSSEPFMTQGQMPNSSMQDMYN
QSPSGAMSNLGMGQRQQFPYGASYDRRHEPYGQQYPGQGPPSGQPPYGGHQPGLYPQQP
NYKRHMDGMYGPPAKRHEGDMYNMQYSSQQQEMYNQYGGSYSGPDRRPIQGQYPYPYSR
ERMQGPGQIQTHGIPPQMMGGPLQSSSSEGPQQNMWAARNDMPYPYQNRQGPGGPTQAP
PYPGMNRTDDMMVPDQRINHESQWPSHVSQRQPYMSSSASMQPITRPPQPSYQTPPSLP
NHISRAPSPASFQRSLENRMSPSKSPFLPSMKMQKVMPTVPTSQVTGPPPQPPPIRREI
TFPPGSVEASQPVLKQRRKITSKDIVTPEAWRVMMSLKSGLLAESTWALDTINILLYDD
STVATFNLSQLSGFLELLVEYFRKCLIDIFGILMEYEVGDPSQKALDHNAARKDDSQSL
ADDSGKEEEDAECIDDDEEDEEDEEEDSEKTESDEKSSIALTAPDAAADPKEKPKQASK
FDKLPIKIVKKNNLFVVDRSDKLGRVQEFNSGLLHWQLGGGDTTEHIQTHFESKMEIPP
RRRPPPPLSSAGRKKEQEGKGDSEEQQEKSIIATIDDVLSARPGALPEDANPGPQTESS
KFPFGIQQAKSHRNIKLLEDEPRSRDETPLCTIAHWQDSLAKRCICVSNIVRSLSFVPG
NDAEMSKHPGLVLILGKLILLHHEHPERKRAPQTYEKEEDEDKGVACSKDEWWWDCLEV
LRDNTLVTLANISGQLDLSAYTESICLPILDGLLHWMVCPSAEAQDPFPTVGPNSVLSP
QRLVLETLCKLSIQDNNVDLILATPPFSRQEKFYATLVRYVGDRKNPVCREMSMALLSN
LAQGDALAARAIAVQKGSIGNLISFLEDGVTMAQYQQSQHNLMHMQPPPLEPPSVDMMC
RAAKALLAMARVDENRSEFLLHEGRLLDISISAVLNSLVASVICDVLFQIGQL
ARID1B-2
SEQ ID NO: 31
MAHNAGAAAAAGTHSAKSGGSEAALKEGGSAAALSSSSSSSAAAAAASSSSSSGPGSAM
ETGLLPNHKLKTVGEAPAAPPHQQHHHHHHAHHHHHHAHHLHHHHALQQQLNQFQQQQQ
QQQQQQQQQQQQQHPISNNNSLGGAGGGAPQPGPDMEQPQHGGAKDSAAGGQADPPGPP
LLSKPGDEDDAPPKMGEPAGGRYEHPGLGALGTQQPPVAVPGGGGGPAAVPEFNNYYGS
AAPASGGPGGRAGPCFDQHGGQQSPGMGMMHSASAAAAGAPGSMDPLQNSHEGYPNSQC
NHYPGYSRPGAGGGGGGGGGGGGGSGGGGGGGGAGAGGAGAGAVAAAAAAAAAAAGGGG
GGGYGGSSAGYGVLSSPRQQGGGMMMGPGGGGAASLSKAAAGSAAGGFQRFAGQNQHPS
GATPTLNQLLTSPSPMMRSYGGSYPEYSSPSAPPPPPSQPQSQAAAAGAAAGGQQAAAG
MGLGKDMGAQYAAASPAWAAAQQRSHPAMSPGTPGPTMGRSQGSPMDPMVMKRPQLYGM
GSNPHSQPQQSSPYPGGSYGPPGPQRYPIGIQGRTPGAMAGMQYPQQQDSGDATWKETF
WLMPPQYGQQGVSGYCQQGQQPYYSQQPQPPHLPPQAQYLPSQSQQRYQPQQDMSQEGY
GTRSQPPLAPGKPNHEDLNLIQQERPSSLPDLSGSIDDLPTGTEATLSSAVSASGSTSS
QGDQSNPAQSPFSPHASPHLSSIPGGPSPSPVGSPVGSNQSRSGPISPASIPGSQMPPQ
PPGSQSESSSHPALSQSPMPQERGFMAGTQRNPQMAQYGPQQTGPSMSPHPSPGGQMHA
GISSFQQSNSSGTYGPQMSQYGPQGNYSRPPAYSGVPSASYSGPGPGMGISANNQMHGQ
GPSQPCGAVPLGRMPSAGMQNRPFPGNMSSMTPSSPGMSQQGGPGMGPPMPTVNRKAQE
AAAAVMQAAANSAQSRQGSFPGMNQSGLMASSSPYSQPMNNSSSLMNTQAPPYSMAPAM
VNSSAASVGLADMMSPGESKLPLPLKADGKEEGTPQPESKSKKSSSSTTTGEKITKVYE
LGNEPERKLWVDRYLTFMEERGSPVSSLPAVGKKPLDLFRLYVCVKEIGGLAQVNKNKK
WRELATNLNVGTSSSAASSLKKQYIQYLFAFECKIERGEEPPPEVFSTGDTKKQPKLQP
PSPANSGSLQGPQTPQSTGSNSMAEVPGDLKPPTPASTPHGQMTPMQGGRSSTISVHDP
FSDVSDSSFPKRNSMTPNAPYQQGMSMPDVMGRMPYEPNKDPFGGMRKVPGSSEPFMTQ
GQMPNSSMQDMYNQSPSGAMSNLGMGQRQQFPYGASYDRRHEPYGQQYPGQGPPSGQPP
YGGHQPGLYPQQPNYKRHMDGMYGPPAKRHEGDMYNMQYSSQQQEMYNQYGGSYSGPDR
RPIQGQYPYPYSRERMQGPGQIQTHGIPPQMMGGPLQSSSSEGPQQNMWAARNDMPYPY
QNRQGPGGPTQAPPYPGMNRTDDMMVPDQRINHESQWPSHVSQRQPYMSSSASMQPITR
PPQPSYQTPPSLPNHISRAPSPASFQRSLENRMSPSKSPFLPSMKMQKVMPTVPTSQVT
GPPPQPPPIRREITFPPGSVEASQPVLKQRRKITSKDIVTPEAWRVMMSLKSGLLAEST
WALDTINILLYDDSTVATFNLSQLSGFLELLVEYFRKCLIDIFGILMEYEVGDPSQKAL
DHNAARKDDSQSLADDSGKEEEDAECIDDDEEDEEDEEEDSEKTESDEKSSIALTAPDA
AADPKEKPKQASKFDKLPIKIVKKNNLFVVDRSDKLGRVQEFNSGLLHWQLGGGDTTEH
IQTHFESKMEIPPRRRPPPPLSSAGRKKEQEGKGDSEEQQEKSIIATIDDVLSARPGAL
PEDANPGPQTESSKFPFGIQQAKSHRNIKLLEDEPRSRDETPLCTIAHWQDSLAKRCIC
VSNIVRSLSFVPGNDAEMSKHPGLVLILGKLILLHHEHPERKRAPQTYEKEEDEDKGVA
CSKDEWWWDCLEVLRDNTLVTLANISGQLDLSAYTESICLPILDGLLHWMVCPSAEAQD
PFPTVGPNSVLSPQRLVLETLCKLSIQDNNVDLILATPPFSRQEKFYATLVRYVGDRKN
PVCREMSMALLSNLAQGDALAARAIAVQKGSIGNLISFLEDGVTMAQYQQSQHNLMHMQ
PPPLEPPSVDMMCRAAKALLAMARVDENRSEFLLHEGRLLDISISAVLNSLVASVICDV
LFQIGQL
ARID1B-3
SEQ ID NO: 32
MAHNAGAAAAAGTHSAKSGGSEAALKEGGSAAALSSSSSSSAAAAAASSSSSSGPGSAM
ETGLLPNHKLKTVGEAPAAPPHQQHHHHHHAHHHHHHAHHLHHHHALQQQLNQFQQQQQ
QQQQQQQQQQQQQHPISNNNSLGGAGGGAPQPGPDMEQPQHGGAKDSAAGGQADPPGPP
LLSKPGDEDDAPPKMGEPAGGRYEHPGLGALGTQQPPVAVPGGGGGPAAVPEFNNYYGS
AAPASGGPGGRAGPCFDQHGGQQSPGMGMMHSASAAAAGAPGSMDPLQNSHEGYPNSQC
NHYPGYSRPGAGGGGGGGGGGGGGSGGGGGGGGAGAGGAGAGAVAAAAAAAAAAAGGGG
GGGYGGSSAGYGVLSSPRQQGGGMMMGPGGGGAASLSKAAAGSAAGGFQRFAGQNQHPS
GATPTLNQLLTSPSPMMRSYGGSYPEYSSPSAPPPPPSQPQSQAAAAGAAAGGQQAAAG
MGLGKDMGAQYAAASPAWAAAQQRSHPAMSPGTPGPTMGRSQGSPMDPMVMKRPQLYGM
GSNPHSQPQQSSPYPGGSYGPPGPQRYPIGIQGRTPGAMAGMQYPQQQMPPQYGQQGVS
GYCQQGQQPYYSQQPQPPHLPPQAQYLPSQSQQRYQPQQDMSQEGYGTRSQPPLAPGKP
NHEDLNLIQQERPSSLPDLSGSIDDLPTGTEATLSSAVSASGSTSSQGDQSNPAQSPFS
PHASPHLSSIPGGPSPSPVGSPVGSNQSRSGPISPASIPGSQMPPQPPGSQSESSSHPA
LSQSPMPQERGEMAGTQRNPQMAQYGPQQTGPSMSPHPSPGGQMHAGISSFQQSNSSGT
YGPQMSQYGPQGNYSRPPAYSGVPSASYSGPGPGMGISANNQMHGQGPSQPCGAVPLGR
MPSAGMQNRPFPGNMSSMTPSSPGMSQQGGPGMGPPMPTVNRKAQEAAAAVMQAAANSA
QSRQGSFPGMNQSGLMASSSPYSQPMNNSSSLMNTQAPPYSMAPAMVNSSAASVGLADM
MSPGESKLPLPLKADGKEEGTPQPESKSKDSYSSQGISQPPTPGNLPVPSPMSPSSASI
SSFHGDESDSISSPGWPKTPSSPKSSSSTTTGEKITKVYELGNEPERKLWVDRYLTFME
ERGSPVSSLPAVGKKPLDLFRLYVCVKEIGGLAQVNKNKKWRELATNLNVGTSSSAASS
LKKQYIQYLFAFECKIERGEEPPPEVFSTGDTKKQPKLQPPSPANSGSLQGPQTPQSTG
SNSMAEVPGDLKPPTPASTPHGQMTPMQGGRSSTISVHDPFSDVSDSSFPKRNSMTPNA
PYQQGMSMPDVMGRMPYEPNKDPFGGMRKVPGSSEPFMTQGQMPNSSMQDMYNQSPSGA
MSNLGMGQRQQFPYGASYDRRHEPYGQQYPGQGPPSGQPPYGGHQPGLYPQQPNYKRHM
DGMYGPPAKRHEGDMYNMQYSSQQQEMYNQYGGSYSGPDRRPIQGQYPYPYSRERMQGP
GQIQTHGIPPQMMGGPLQSSSSEGPQQNMWAARNDMPYPYQNRQGPGGPTQAPPYPGMN
RTDDMMVPDQRINHESQWPSHVSQRQPYMSSSASMQPITRPPQPSYQTPPSLPNHISRA
PSPASFQRSLENRMSPSKSPFLPSMKMQKVMPTVPTSQVTGPPPQPPPIRREITFPPGS
VEASQPVLKQRRKITSKDIVTPEAWRVMMSLKSGLLAESTWALDTINILLYDDSTVATF
NLSQLSGFLELLVEYFRKCLIDIFGILMEYEVGDPSQKALDHNAARKDDSQSLADDSGK
EEEDAECIDDDEEDEEDEEEDSEKTESDEKSSIALTAPDAAADPKEKPKQASKFDKLPI
KIVKKNNLFVVDRSDKLGRVQEFNSGLLHWQLGGGDTTEHIQTHFESKMEIPPRRRPPP
PLSSAGRKKEQEGKGDSEEQQEKSIIATIDDVLSARPGALPEDANPGPQTESSKFPFGI
QQAKSHRNIKLLEDEPRSRDETPLCTIAHWQDSLAKRCICVSNIVRSLSFVPGNDAEMS
KHPGLVLILGKLILLHHEHPERKRAPQTYEKEEDEDKGVACSKDEWWWDCLEVLRDNTL
VTLANISGQLDLSAYTESICLPILDGLLHWMVCPSAEAQDPFPTVGPNSVLSPQRLVLE
TLCKLSIQDNNVDLILATPPFSRQEKFYATLVRYVGDRKNPVCREMSMALLSNLAQGDA
LAARAIAVQKGSIGNLISFLEDGVTMAQYQQSQHNLMHMQPPPLEPPSVDMMCRAAKAL
LAMARVDENRSEFLLHEGRLLDISISAVLNSLVASVICDVLFQIGQL
ARID1B-4
SEQ ID NO: 33
MPPQPPGSQSESSSHPALSQSPMPQERGFMAGTQRNPQMAQYGPQQTGPSMSPHPSPGG
QMHAGISSFQQSNSSGTYGPQMSQYGPQGNYSRPPAYSGVPSASYSGPGPGMGISANNQ
MHGQGPSQPCGAVPLGRMPSAGMQNRPFPGNMSSMTPSSPGMSQQGGPGMGPPMPTVNR
KAQEAAAAVMQAAANSAQSRQGSFPGMNQSGLMASSSPYSQPMNNSSSLMNTQAPPYSM
APAMVNSSAASVGLADMMSPGESKLPLPLKADGKEEGTPQPESKSKKSSSSTTTGEKIT
KVYELGNEPERKLWVDRYLTFMEERGSPVSSLPAVGKKPLDLFRLYVCVKEIGGLAQVN
KNKKWRELATNLNVGTSSSAASSLKKQYIQYLFAFECKIERGEEPPPEVFSTGDTKKQP
KLQPPSPANSGSLQGPQTPQSTGSNSMAEVPGDLKPP
TPASTPHGQMTPMQGGRSSTISVHDPFSDVSDSSFPKRNSMTPNAPYQQGMSMPDVMGR
MPYEPNKDPFGGMRKVPGSSEPFMTQGQMPNSSMQDMYNQSPSGAMSNLGMGQRQQFPY
GASYDRRHEPYGQQYPGQGPPSGQPPYGGHQPGLYPQQPNYKRHMDGMYGPPAKRHEGD
MYNMQYSSQQQEMYNQYGGSYSGPDRRPIQGQYPYPYSRERMQGPGQIQTHGIPPQMMG
GPLQSSSSEGPQQNMWAARNDMPYPYQNRQGPGGPTQAPPYPGMNRTDDMMVPDQRINH
ESQWPSHVSQRQPYMSSSASMQPITRPPQPSYQTPPSLPNHISRAPSPASFQRSLENRM
SPSKSPFLPSMKMQKVMPTVPTSQVTGPPPQPPPIRREITFPPGSVEASQPVLKQRRKI
TSKDIVTPEAWRVMMSLKSGLLAESTWALDTINILLYDDSTVATFNLSQLSGFLELLVE
YFRKCLIDIFGILMEYEVGDPSQKALDHNAARKDDSQSLADDSGKEEEDAECIDDDEED
EEDEEEDSEKTESDEKSSIALTAPDAAADPKEKPKQASKFDKLPIKIVKKNNLFVVDRS
DKLGRVQEFNSGLLHWQLGGGDTTEHIQTHFESKMEIPPRRRPPPPLSSAGRKKEQEGK
GDSEEQQEKSIIATIDDVLSARPGALPEDANPGPQTESSKFPFGIQQAKSHRNIKLLED
EPRSRDETPLCTIAHWQDSLAKRCICVSNIVRSLSFVPGNDAEMSKHPGLVLILGKLIL
LHHEHPERKRAPQTYEKEEDEDKGVACSKDEWWWDCLEVLRDNTLVTLANISGQLDLSA
YTESICLPILDGLLHWMVCPSAEAQDPFPTVGPNSVLSPQRLVLETLCKLSIQDNNVDL
ILATPPFSRQEKFYATLVRYVGDRKNPVCREMSMALLSNLAQGDALAARAIAVQKGSIG
NLISFLEDGVTMAQYQQSQHNLMHMQPPPLEPPSVDMMCRAAKALLAMARVDENRSEFL
LHEGRLLDISISAVLNSLVASVICDVLFQIGQL
SS18-1
SEQ ID NO: 34
MSVAFAAPRQRGKGEITPAAIQKMLDDNNHLIQCIMDSQNKGKTSECSQYQQMLHTNLV
YLATIADSNQNMQSLLPAPPTQNMPMGPGGMNQSGPPPPPRSHNMPSDGMVGGGPPAPH
MQNQMNGQMPGPNHMPMQGPGPNQLNMTNSSMNMPSSSHGSMGGYNHSVPSSQSMPVQN
QMTMSQGQPMGNYGPRPNMSMQPNQGPMMHQQPPSQQYNMPQGGGQHYQGQQPPMGMMG
QVNQGNHMMGQRQIPPYRPPQQGPPQQYSGQEDYYGDQYSHGGQGPPEGMNQQYYPDGH
NDYGYQQPSYPEQGYDRPYEDSSQHYYEGGNSQYGQQQDAYQGPPPQQGYPPQQQQYPG
QQGYPGQQQGYGPSQGGPGPQYPNYPQGQGQQYGGYRPTQPGPPQPPQQRPYGYDQGQY
GNYQQ
SS18-2
SEQ ID NO: 35
MSVAFAAPRQRGKGEITPAAIQKMLDDNNHLIQCIMDSQNKGKTSECSQYQQMLHTNLV
YLATIADSNQNMQSLLPAPPTQNMPMGPGGMNQSGPPPPPRSHNMPSDGMVGGGPPAPH
MQNQMNGQMPGPNHMPMQGPGPNQLNMTNSSMNMPSSSHGSMGGYNHSVPSSQSMPVQN
QMTMSQGQPMGNYGPRPNMSMQPNQGPMMHQQPPSQQYNMPQGGGQHYQGQQPPMGMMG
QVNQGNHMMGQRQIPPYRPPQQGPPQQYSGQEDYYGDQYSHGGQGPPEGMNQQYYPDGN
SQYGQQQDAYQGPPPQQGYPPQQQQYPGQQGYPGQQQGYGPSQGGPGPQYPNYPQGQGQ
QYGGYRPTQPGPPQPPQQRPYGYDQGQYGNYQQ

### EXAMPLES

### Example 1: Cell Growth Suppression Experiment

The cytotoxic activity of a CBP/P300 inhibitor on SMARCB1 deficient cells, i.e., G-401 cells (malignant rhabdoid tumor derived), G-402 cells (malignant rhabdoid tumor derived), and CHLA-06-ATRT cells (atypical teratoid/rhabdoid tumor derived), and SMARCB1 wild-type cells, i.e., 786-O cells (renal cancer derived) was compared.

The G-401 cells, G-402 cells, CHLA-06-ATRT cells, and 786-O cells were obtained from the American Type Culture Collection (ATCC). G-401 cells and G-402 cells were cultured under the conditions of 5% CO₂ at 37° C. in a 10% fetal bovine serum and 1% penicillin/streptomycin-containing McCoy's 5A medium. CHLA-06-ATRT cells were cultured under the conditions of 5% CO₂ at 37° C. in a 10% B-27 supplement, 20 ng/mL EGF, 20 ng/mL FGF, and 1% penicillin/streptomycin-containing DMEM:F12 medium. 786-0 cells were cultured under the conditions of 5% CO₂ at 37° C. in a 10% fetal bovine serum and 1% penicillin/streptomycin-containing RPMI-1640 medium.

A 384-well plate was seeded at 500 cells per well. After one day from seeding, compounds 1 to 19 (Table 47) were added so that the final concentration of DMSO would be 0.1%, and the cells were cultured for 3 days. After completion of culture, the cell survival rate was measured using CellTiter-Glo Luminescent Cell Viability Assay (Promega, G7570). The IC₅₀ value corresponding to the concentration of evaluated compounds at which a cell growth suppression ratio of 50% is exhibited was calculated from a cell survival curve. The results are shown in Table 48. Tests in which the cell survival rate was higher than 50% at 10 μM that is the maximum treatment concentration of each compound are indicated with an IC₅₀ value of “>10 μM”, and the cell survival rate (%) at treatment with 10 μM is indicated within the parenthesis. The compounds subjected to this experiment are shown in Table 21.

TABLE 47-1

| Compound | Structure |
|---|---|
| 1 (Single diastereomer) | 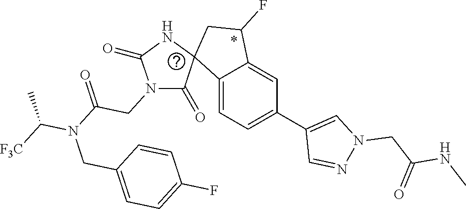 [SMILES: CNC(=O)Cn1cc(-c2ccc3c(c2)C(F)CC32NC(=O)N(CC(=O)N(Cc3ccc(F)cc3)[C@@H](C)C(F)(F)F)C2=O)cn1] |
| 2 (Single diastereomer) | 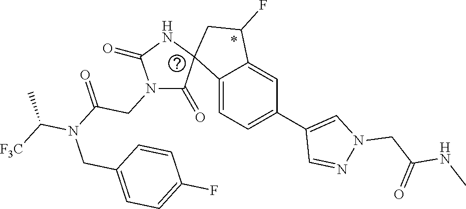 [SMILES: CNC(=O)Cn1cc(-c2ccc3c(c2)C(F)CC32NC(=O)N(CC(=O)N(Cc3ccc(F)cc3)[C@@H](C)C(F)(F)F)C2=O)cn1] |
| 3 (Single diastereomer) | 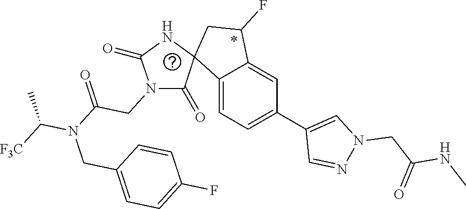 [SMILES: CNC(=O)Cn1cc(-c2ccc3c(c2)C(F)CC32NC(=O)N(CC(=O)N(Cc3ccc(F)cc3)[C@@H](C)C(F)(F)F)C2=O)cn1] |
| 4 (Single diastereomer) | 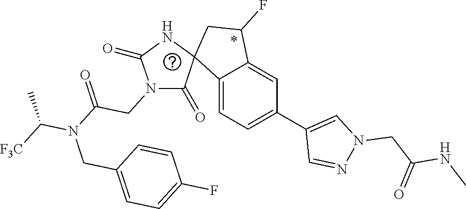 [SMILES: CNC(=O)Cn1cc(-c2ccc3c(c2)C(F)CC32NC(=O)N(CC(=O)N(Cc3ccc(F)cc3)[C@@H](C)C(F)(F)F)C2=O)cn1] |
| 5 | 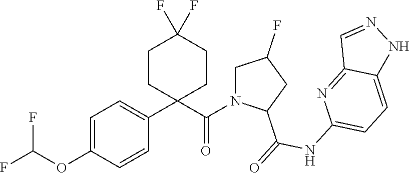 [SMILES: O=C(Nc1ccc2[nH]ncc2n1)C1CC(F)CN1C(=O)C1(c2ccc(OC(F)F)cc2)CCC(F)(F)CC1] |
| 6 | 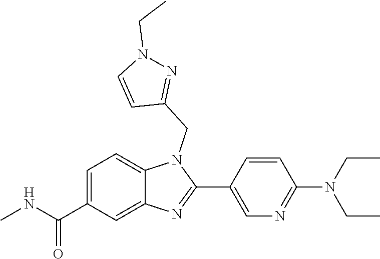 [SMILES: CCN(CC)c1ccc(-c2nc3cc(C(=O)NC)ccc3n2Cc2ccn(CC)n2)cn1] |
| 7 | 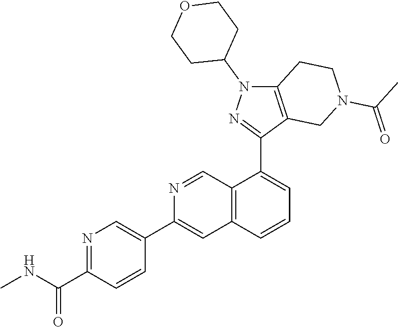 [SMILES: CNC(=O)c1ccc(-c2cc3cccc(-c4nn(C5CCOCC5)c5c4CN(C(C)=O)CC5)c3cn2)cn1] |
| 8 | 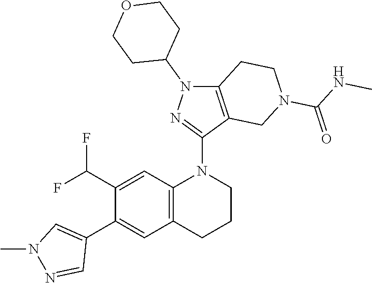 |

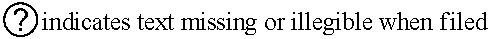

TABLE 47-2

| | |
|---|---|
| 9 | 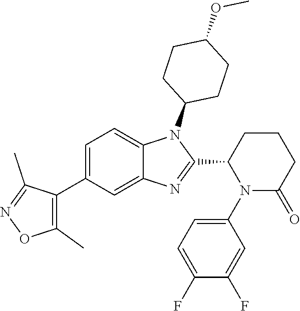 [SMILES: CO[C@H]1CC[C@H](n2c([C@@H]3CCCC(=O)N3c3ccc(F)c(F)c3)nc3cc(-c4c(C)noc4C)ccc32)CC1] |
| 10 | 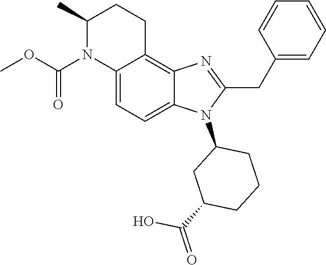 [SMILES: COC(=O)N1c2ccc3c(nc(Cc4ccccc4)n3[C@H]3CCC[C@H](C(=O)O)C3)c2CC[C@@H]1C] |
| 11 | 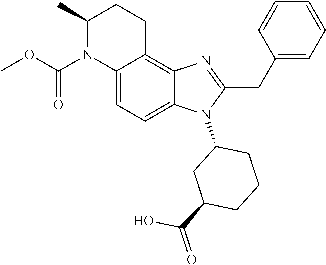 [SMILES: COC(=O)N1c2ccc3c(nc(Cc4ccccc4)n3[C@@H]3CCC[C@@H](C(=O)O)C3)c2CC[C@@H]1C] |
| 12 | 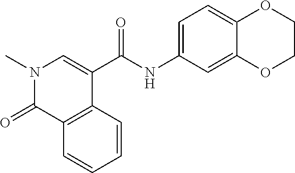 [SMILES: Cn1cc(C(=O)Nc2ccc3c(c2)OCCO3)c2ccccc2c1=O] |
| 13 | 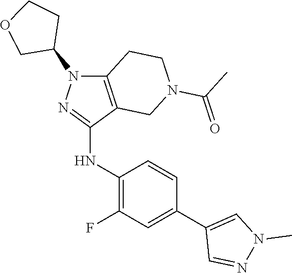 [SMILES: CC(=O)N1CCc2c(c(Nc3ccc(-c4cnn(C)c4)cc3F)nn2[C@@H]2CCOC2)C1] |
| 14 | 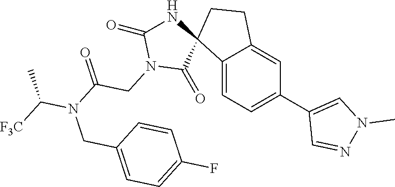 [SMILES: C[C@H](N(Cc1ccc(F)cc1)C(=O)CN1C(=O)N[C@]2(CCc3cc(-c4cnn(C)c4)ccc32)C1=O)C(F)(F)F] |
| 15 | 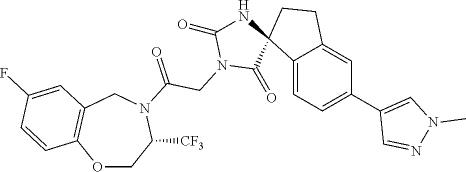 [SMILES: Cn1cc(-c2ccc3c(c2)CC[C@]32NC(=O)N(CC(=O)N3Cc4cc(F)ccc4OC[C@H]3C(F)(F)F)C2=O)cn1] |
| 16 | 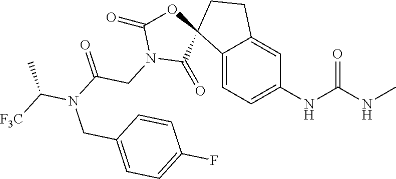 [SMILES: CNC(=O)Nc1ccc2c(c1)CC[C@]21OC(=O)N(CC(=O)N(Cc2ccc(F)cc2)[C@@H](C)C(F)(F)F)C1=O] |

TABLE 47-3

| | |
|---|---|
| 17 | 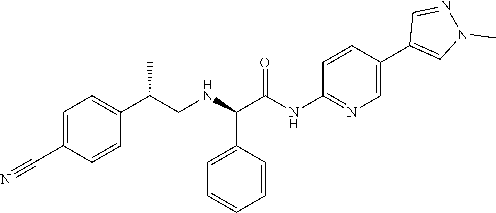 [SMILES: C[C@H](CN[C@@H](C(=O)Nc1ccc(-c2cnn(C)c2)cn1)c1ccccc1)c1ccc(C#N)cc1] |
| 18 | 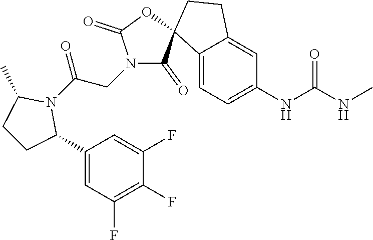 [SMILES: CNC(=O)Nc1ccc2c(c1)CC[C@@]21OC(=O)N(CC(=O)N2[C@@H](C)CC[C@H]2c2cc(F)c(F)c(F)c2)C1=O] |
| 19 | 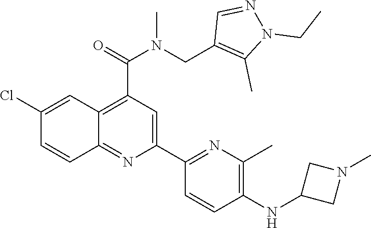 [SMILES: CCn1ncc(CN(C)C(=O)c2cc(-c3ccc(NC4CN(C)C4)c(C)n3)nc3ccc(Cl)cc23)c1C] |
| SGC-CB P30 | 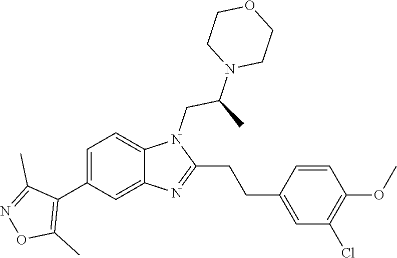 [SMILES: COc1ccc(CCc2nc3cc(-c4c(C)noc4C)ccc3n2C[C@H](C)N2CCOCC2)cc1Cl] |

TABLE 48

| | IC₅₀ (μM) | | | |
|---|---|---|---|---|
| | | | CHLA-06- | |
| Compound | G-401 | G-402 | ATRT | 786-O |
| 1 | 2.7 | — | — | >10 (104%) |
| 2 | >10 (62%) | — | — | >10 (108%) |
| 3 | 4.9 | — | — | >10 (107%) |
| 4 | 0.018 | 0.93 | 0.18 | >10 (93%) |
| 5 | 0.44 | — | — | >10 (102%) |
| 6 | 0.26 | — | — | >10 (83%) |
| 7 | >10 (72%) | — | — | >10 (96%) |
| 8 | >10 (65%) | — | — | >10 (92%) |
| 9 | 2.6 | — | — | >10 (83%) |
| 10 | 1.8 | — | — | >10 (82%) |
| 11 | >10 (53%) | — | — | >10 (93%) |
| 12 | >10 (83%) | — | — | >10 (104%) |
| 13 | >10 (78%) | — | — | >10 (104%) |
| 14 | 0.15 | — | 1.3 | >10 (74%) |
| 15 | 0.041 | — | 0.42 | >10 (51%) |
| 16 | 0.35 | 8.9 | — | >10 (94%) |
| 17 | 0.73 | — | — | >10 (94%) |
| 18 | 0.068 | — | — | — |
| 19 | 0.08 | — | — | >10 (69%) |

As shown in Table 48, GBP/P300 inhibitors, i.e., compounds 1, 3, 4, 5, 6, 9, 10, 14, 15, 16, 17, 18, and 19 exhibited a selective and potent Cell growth suppression effect on SMARCB1 deficient cells, i.e., G-401 cells, G-402 cells, and CHLA-06-ATRT cells. Meanwhile, a cell growth suppression effect was hardly exhibited on human renal cancer cell line and SMARCB1 expressing 786-O cells.

In view of the above, CBP/P300 inhibitors exhibited a selective and potent cell growth suppression effect on SMARCB1 deficient malignant rhabdoid tumor cells and atypical teratoid/rhabdoid tumor cells.

### Example 2: Experiment Evaluating Selectivity in Cell Growth Suppression

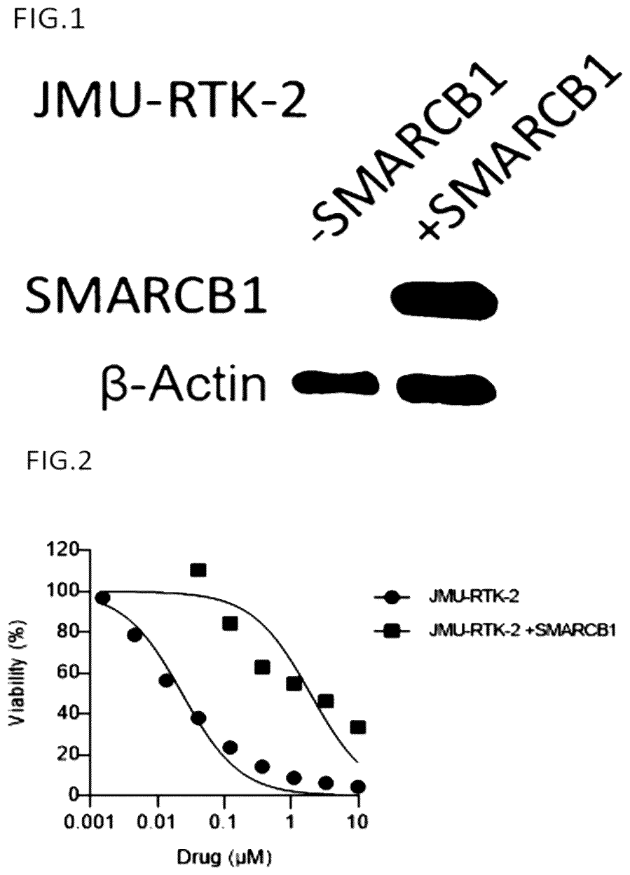

An SMARCB1 overexpressing vector (Precision LentiORF Human SMARCB1 with Stop Codon, Dharmacon, OHS5897-202617080) was introduced into SMARCB1 deficient cells, i.e., JMU-RTK-2 cells (malignant rhabdoid tumor derived), with a lentivirus. After selection with the agent Blasticidine, the cells were cloned to create JMU-RTK-2+SMARCB1 cells (FIG. 1). The cytotoxic activity of a CBP/P300 inhibitor on JMU-RTK-2 cells and JMU-RTK-2+SMARCB1 cells was compared.

JMU-RTK-2 cells and JMU-RTK-2+SMARCB1 cells were seeded on a 96-well plate at 500 to 2000 cells/well. After one day from seeding, compounds 1 to 16 were added so that the final concentration of DMSO would be 0.1%, and the cells were cultured for 6 days. After completion of culture, the cell survival rate was measured using CellTiter-Glo Luminescent Cell Viability Assay (Promega, G7570). The IC₅₀ value corresponding to the concentration of evaluated compounds at which a cell growth suppression ratio of 50% is exhibited was calculated from a cell survival curve. The IC₅₀ value of JMU-RTK-2+SMARCB1 cells with respect to the IC₅₀ value of JMU-RTK-2 cells was calculated as a Selective Index for SMARCB1 deficient cells. The results are shown in Table 49.

TABLE 49

| | JMU-RTK-2 | JMU-RTK-2 + | |
|---|---|---|---|
| | IC₅₀ | SMARCB1 | Selective |
| Compound | (μM) | IC₅₀ (μM) | Index |
| 1 | 2.5 | 27.3 | 10.9 |
| 2 | 4.8 | 20.1 | 4.2 |
| 3 | 2.6 | 26.3 | 10.2 |
| 4 | 0.0099 | 1.4 | 144.2 |
| 5 | 0.20 | 8.8 | 45.3 |
| 6 | 0.055 | 1.3 | 23.5 |
| 7 | 2.3 | 7.5 | 3.3 |
| 8 | 0.9 | 9.1 | 10.2 |
| 9 | 0.7 | 3.7 | 5.6 |
| 10 | 1.6 | 5.3 | 3.2 |
| 11 | 2.0 | 5.8 | 3.0 |
| 12 | 3.9 | 14.9 | 3.9 |
| 13 | 2.8 | 8.1 | 2.9 |
| 14 | 0.012 | 5.2 | 431 |
| 15 | 0.0038 | 3.4 | 912 |
| 16 | 0.13 | 13 | 98 |

As shown in Table 49, CBP/P300 inhibitors, i.e., compounds 1 to 16, exhibited a selective and potent cell growth suppression effect on SMARCB1 deficient JMU-RTK-2 cells. Selective Index, which is the ratio of the cell growth suppression effects of SMARCB1 expressing cells and SMARCB1 deficient cells, exhibited a value of 1 or greater.

This demonstrated that CBP/P300 inhibitors have a selective cell growth suppression effect on SMARCB1 deficient malignant rhabdoid tumor cells.

### Example 3: Experiment Comparing Sensitivity Depending on the Presence/Absence of SMARCB1

Cytotoxic activity of a CBP/P300 inhibitor on SMARCB1 deficient cells, i.e., G-401 cells (malignant rhabdoid tumor derived), G-402 cells (malignant rhabdoid tumor derived), JMU-RTK-2 cells (malignant rhabdoid tumor derived), and HS-EU-1 cells (epithelioid sarcoma derived), and SMARCB1 wild-type cells, i.e., 786-O cells (renal cancer derived), VMRC-RCZ cells (renal cancer derived), Caki-1 cells (renal cancer derived), H446 cells (lung cancer derived), ES2 cells (ovarian cancer derived), H460 cells (lung cancer derived), H2228 cells (lung cancer derived), HEK293T cells (normal kidney tissue derived), and H358 cells (lung cancer derived) was compared.

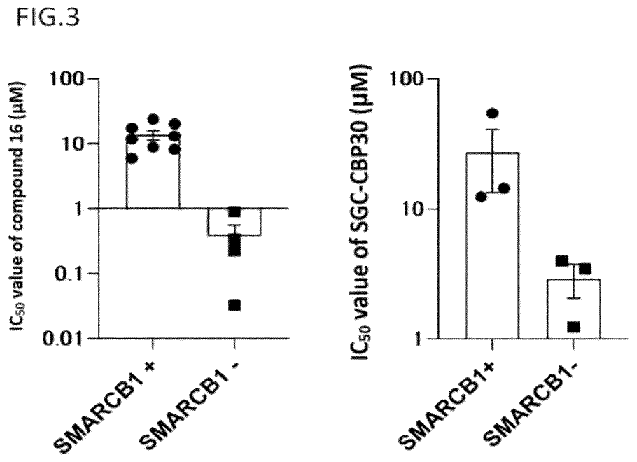

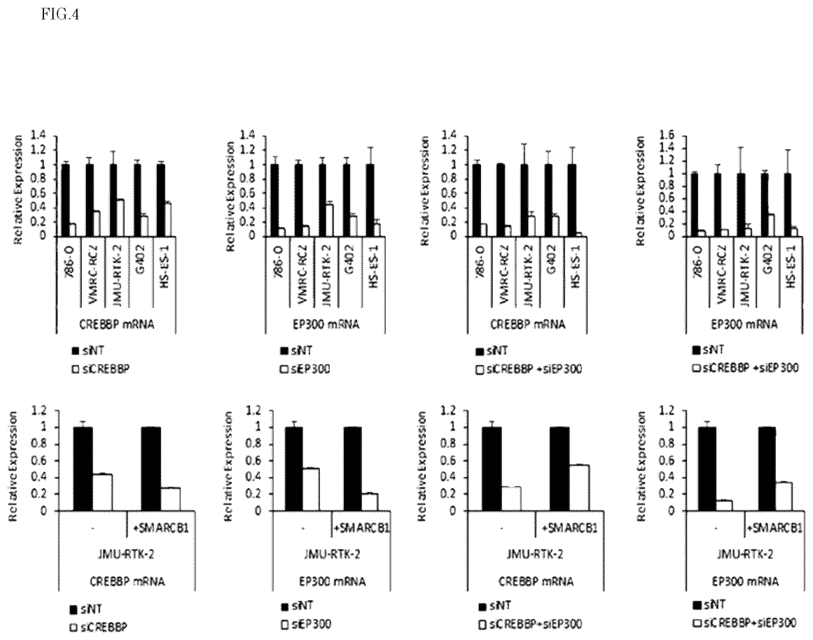

G-402 cells, JMU-RTK-2 cells, and HS-ES-1 cells, 786-0 cells, VMRC-RCZ cells, and Caki-1 cells were seeded on a 96-well plate at 500 to 2000 cells per well. After one day from seeding, SGC-CBP30, which is a compound known as a BRD inhibitor, was added so that the final concentration of DMSO would be 0.1%, and the cells were cultured for 6 days. G-401 cells, G-402 cells, JMU-RTK-2 cells and HS-ES-1 cells, and H446 cells, ES2 cells, H460 cells, H2228 cells, HEK293T cells, VMRC-RCZ cells, and H358 cells were also seeded on a 96-well plate at 500 to 2000 cells per well. After one day from seeding, compound 16 was added so that the final concentration of DMSO would be 0.1%, and the cells were cultured for 6 days. After completion of culture, the cell survival rate was measured using CellTiter-Glo Luminescent Cell Viability Assay (Promega, G7570). The IC₅₀ value corresponding to the concentration of evaluated compounds at which a cell growth suppression ratio of 50% is exhibited was calculated from a cell survival curve. The IC₅₀ value of the SMARCB1 deficient cell group was compared with the IC₅₀ value of the SMARCB1 wild-type cell group (FIG. 3).

As shown in FIG. 3, the IC₅₀ value of compound 16 and SGC-CBP30 for the SMARCB1 deficient cell group was less than the IC₅₀ value for the SMARCB1 wild-type cell group. This demonstrated that a CBP/P300 inhibitor has a selective cell growth suppression effect on SMARCB1 deficient malignant rhabdoid tumor cells and epithelioid sarcoma cells.

### Example 4: Experiment on Suppression of Expression by siRNAR

SMARCB1 deficient cells, i.e., G-402 cells (malignant rhabdoid tumor derived), JMU-RTK-2 cells (malignant rhabdoid tumor derived), and HS-ES-1 cells (epithelioid sarcoma derived), SMARCB1 wild-type cells, i.e., 786-0 cells (renal cancer derived) and VMRC-RCZ cells (renal cancer derived), and JMU-RTK-2+SMARCB1 cells which are JMU-RTK-2 cells with overexpression of SMARCB1 were transfected with siRNA of a gene CREBBP encoding CBP (Dharmacon, L-003477-00-0005, hereinafter siCREBBP) and/or siRNA of gene EP300 encoding P300 (Dharmacon, L-003486-00-0005, hereinafter siEP300) to suppress expression of CREBBP and/or EP300.

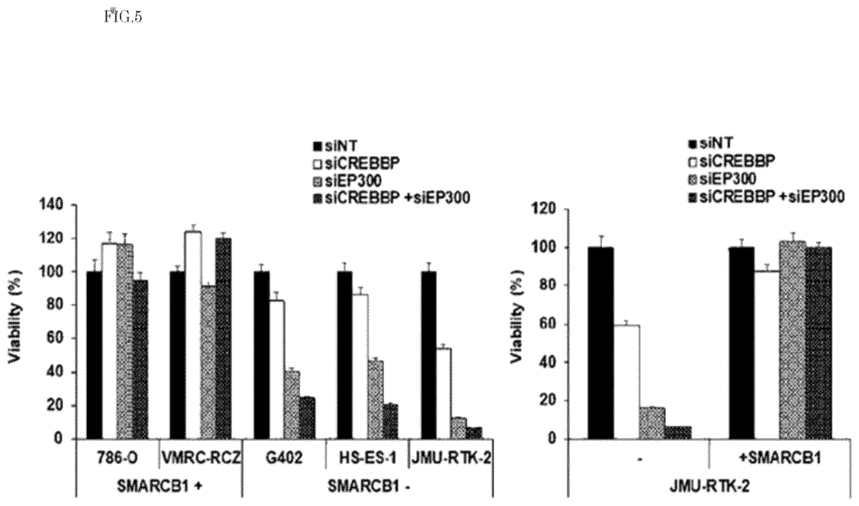

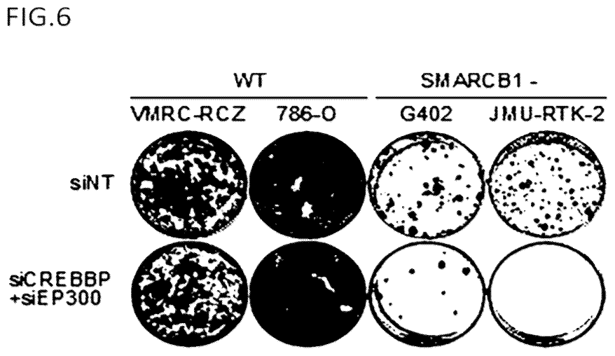

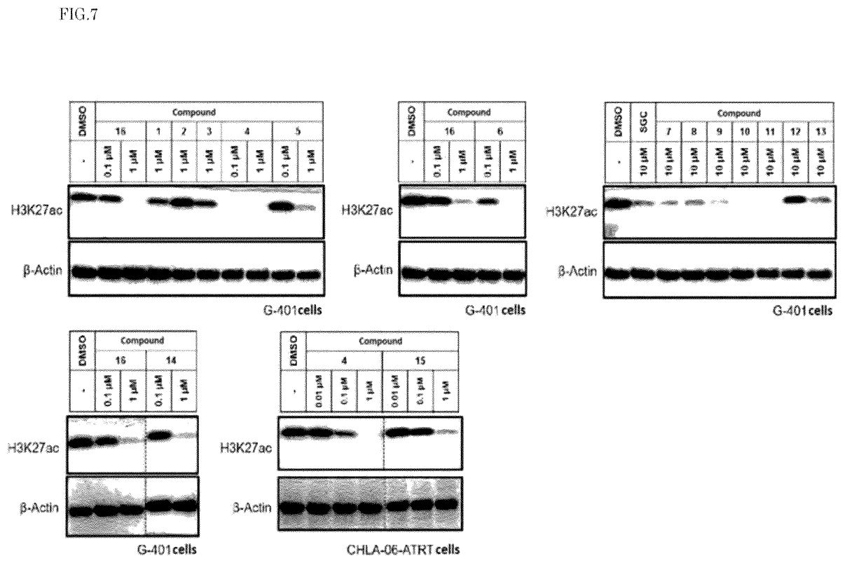

100000 cells were seeded on a 24-well plate per well, and 50 nM of siRNA and Lipofectamine RNAiMAX Transfection Reagent (ThermoFisher, 13778030) was added. After one day, the culture medium was exchanged. After another day, cells were subcultured, and 50 nM of siRNA and Lipofectamine RNAiMAX Transfection Reagent were added again. After another day, the culture medium was exchanged. RNA was extracted from the cells. cDNA was prepared using a reverse transcriptase. The amount of expression of CREBBP and/or EP300 was checked by quantitative PCR (FIG. 4).

<Measurement of cell growth capability> A day after exchanging the culture medium, 500 to 2000 cells were seeded on a 96-well plate per well, and the cells were cultured for 7 days. After completion of culture, the cell survival rate was measured using CellTiter-Glo Luminescent Cell Viability Assay (Promega, G7570) (FIG. 5).

<Observation of colony formation capability> A day after exchanging the culture medium, 500 cells were seeded on a 6-well plate per well, and the cells were cultured for 14 days. After completion of culture, the culture medium was removed. After treatment with a 0.05% Crystal violet/50% methanol solution for 10 minutes, the solution was removed and a picture was taken (FIG. 6).

As shown in FIG. 4, it was confirmed that the amount of mRNA of CREBBP and/or EP300 was reduced and expression was suppressed by treating each cell with siEP300 and/or siCREBBP. Significant decrease in cell survival rate (FIG. 5) and decrease in colony forming capability (FIG. 6) were observed by suppressing the expression of CREBBP and/or EP300 in SMARCB1 deficient cells, i.e., G-402 cells, JMU-RTK-2 cells, and HS-ES-1 cells. This demonstrated that suppression of expression of CBP/P300 has a selective cell growth suppression effect and colony formation capability suppressing effect on SMARCB1 deficient malignant rhabdoid tumor cells and epithelioid sarcoma cells.

### Example 5: HAT Activity Inhibiting Experiment

SensoLyte HAT (p300) Assay Kit (ANASPEC, AS-72172) was used to assess the HAT activity inhibiting capability of a HAT inhibitor. Specifically, 10 μL of compounds 1 to 6 and 14 to 19 diluted with an assay buffer was added to 10 μL of a recombinant p300 solution diluted 10-fold with an assay buffer, and the solution was incubated for 10 minutes at room temperature. 10 μL of acetyl-CoA solution diluted 10-fold with an assay buffer and 20 μL of histone H3 peptide diluted 10-fold with an assay buffer were added thereto, and the mixture was incubated for 30 minutes at 37° C. 50 μL of Stop Solution was added to quench the reaction. 100 μL of p300 Developer solution diluted 50-fold with an assay buffer was added, and the mixture was incubated for 30 minutes at room temperature under shaded conditions. 513 nm fluorescence when irradiated with 389 nm excitation light was measured using a multiplate reader. The IC₅₀ value corresponding to the concentration of a compound at which 50% oxygen reaction inhibition ratio is exhibited was calculated based on the measured fluorescence intensity. The results are shown in Table 50.

TABLE 50

| | HAT activity inhibition |
|---|---|
| Compound | IC₅₀ (μM) |
| 1 | 0.89 |
| 2 | 13.08 |
| 3 | 1.16 |
| 4 | 0.0061 |
| 5 | 0.10 |
| 6 | 0.16 |
| 14 | 0.070 |
| 15 | 0.048 |
| 16 | 0.25 |
| 17 | 0.28 |
| 18 | 0.031 |
| 19 | 0.017 |

As shown in Table 50, compounds 1 to 6 and 14 to 19 were confirmed to inhibit the function of HAT.

### Example 6: BRD Inhibition Experiment

BRD function inhibiting capacity of a BRD inhibitor was evaluated by using a CBP bromodomain TR-FRET Assay Kit (Cayman, 600850). Specifically, 5 μL of compounds 7 to 13 or BRD inhibitor which is SGC-CBP30 diluted with an assay buffer was added to 10 μL of CBP bromodomain Europium Chelate prepared with an assay buffer, and the mixture was incubated for 15 minutes at room temperature under shaded conditions. 5 μL of CBP bromodomain Ligand/APC Acceptor Mixture prepared with an assay buffer was added thereto, and the mixture was incubated for 1 hour at room temperature under shaded conditions. 620 nm and 665 nm fluorescence when irradiated with 320 run excitation light were measured by using a multiplate reader. The IC₅₀ value corresponding to the concentration of a compound at which 50% BRD function inhibition ratio is exhibited was calculated based on the measured fluorescence intensity. The results are shown in Table 51. Tests in which the BRD function inhibition ratio was 50% or greater at 0.025 μM that is the minimum treatment concentration of each compound are indicated with an IC₅₀ value of “<0.025 μM”, and the BRD function inhibition ratio (%) at treatment with 0.025 μM is indicated within the parenthesis.

TABLE 51

| | BRD function inhibition |
|---|---|
| Compound | IC₅₀ (μM) |
| 7 | <0.025 (96.1%) |
| 8 | <0.025 (95.0%) |
| 9 | <0.025 (89.7%) |
| 10 | 0.026 |
| 11 | <0.025 (95.1%) |
| 12 | <0.025 (88.5%) |
| 13 | <0.025 (89.9%) |
| SGC-CBP30 | 0.30 |

As shown in Table 51, compounds 7 to 13 and SGC-CBP30 were confirmed to inhibit BRD function.

### Example 7: Intracellular Histone H3K27 Acetylation Inhibition Experiment

G-401 cells (malignant rhabdoid tumor derived) and CHLA-06-ATRT cells (atypical teratoid/rhabdoid tumor derived) were seeded on a 6-well plate at 500000 cells per well. After one day from seeding, compounds 1 to 16 or BRD inhibitor SGC-CBP30 (denoted as “SGC” in the figures) were added so that the final concentration of DMSO would be 0.1%, and the cells were cultured for 24 hours. After completion of culture, the cells were detached through trypsin treatment. The culture was centrifuged and the supernatant was removed to recover cell pellets. 150 μL of RIPA buffer was added to the cell pellets to lyse the cells to extract the entire protein. Acetylation of histone H3K27 was detected by Western blotting. An anti-acetylation H3K27 antibody (Cell Signaling Technology, 8173) was used for the detection of acetylated H3K27 (FIG. 7).

As shown in FIG. 7, compounds 1 to 16 and BRD inhibitor SGC-CBP30 reduced treatment concentration dependent intracellular histone H3K27 acetylation. In view of the above, compounds 1 to 16 and SGC-CBP30 inhibited histone acetyltransferase activity, which is the function of CBP/P300.

### Example 8: Cell Growth Suppression Experiment

Cytotoxic activity of a CBP/P300 on SMARCA2/A4 deficient cells, i.e., H23 cells (pulmonary adenocarcinoma derived), TOV112D cells (small cell ovarian cancer derived), and DMS114 cells (small cell lung cancer derived), and SMARCA2/A4 wild-type cells, i.e., H460 cells (pulmonary adenocarcinoma derived) was compared.

H23 cells, TOV112D cells, DMS114 cells, and H460 cells were cultured under 5% CO₂ conditions at 37° C. in a 10% fetal bovine serum and 1% penicillin/streptomycin-containing DMEM-F12 medium.

Cells were seed on a 96-well plate at 250 to 500 cells per well. After one day from seeding, compounds % to 19 (Table 47) were added so that the final concentration of DMSO would be 0.1% and the cells were cultured for 6 days. After completion of culture, the cell survival rate was measured using CellTiter-Glo Luminescent Cell Viability Assay (Promega, G770). The value corresponding to the concentration of evaluated compounds at which a cell growth suppression ratio of 50% is exhibited was calculated from a cell, survival curve. The results are shown in Table 52. For tests in which the IC₅₀ value is “>10 μM”, the cell survival rate (%) at treatment with 10 μM is indicated within the parenthesis.

TABLE 52

| | IC₅₀ (μM) | | | |
|---|---|---|---|---|
| Compound | H23 | DMS114 | TOV112D | H460 |
| 1 | >10 (52%) | 4.83 | >10 (44%) | >10 (104%) |
| 2 | >10 (99%) | >10 (67%) | >10 (96%) | >10 (100%) |
| 3 | >10 (49%) | 3.438 | >10 (43%) | >10 (105%) |
| 4 | 0.2483 | 0.03737 | 0.1419 | >10 (78%) |
| 5 | 1.418 | 0.4084 | 1.498 | >10 (99%) |
| 6 | 1.129 | 0.1375 | 0.7286 | >10 (92%) |
| 7 | >10 (95%) | >10 (74%) | >10 (107%) | >10 (104%) |
| 8 | >10 (77%) | 4.008 | >10 (91%) | >10 (101%) |
| 9 | 5.606 | 1.098 | >10 (51%) | >10 (94%) |
| 10 | >10 (53%) | 1.43 | >10 (84%) | >10 (95%) |
| 11 | >10 (75%) | 3.729 | >10 (93%) | >10 (99%) |
| 12 | >10 (97%) | >10 (86%) | >10 (99%) | >10 (100%) |
| 13 | >10 (92%) | >10 (64%) | >10 (121%) | >10 (102%) |
| 14 | 1.256 | 0.3296 | 1.023 | >10 (89%) |
| 15 | 0.4069 | 0.09091 | 0.3092 | >10 (78%) |
| 16 | 0.8669 | 0.2798 | 0.4353 | >10 (62%) |
| 17 | 3.88 | 1.185 | 2.863 | >10 (90%) |
| 18 | 1.059 | 0.2603 | 0.936 | >10 (93%) |
| 19 | 0.6921 | 0.1254 | 0.3106 | >10 (87%) |

As shown in Table 52, CBP/P300 inhibitors, i.e., compounds 1, 2, 3, 4, 5, 6, 9, 10, 11, 14, 15, 16, 17, 18 and 19 exhibited a particularly selective and potent cell growth suppression effect on SMARCA2/A4 deficient cells, i.e., H23 cells, DMS114 cells, and TOV112D cells. Meanwhile, hardly any cell growth suppression effect was exhibited on SMARCA2/A4 expressing H460 cells. Compounds 2, 7, 8, 12 and 13 exhibited a cell growth suppression effect on DMS114 cells.

This demonstrated that a CBP/P300 inhibitor has a selective and potent cell growth suppression effect on SMARCA2/A4 deficient pulmonary adenocarcinoma cells, small cell ovarian cancer cells, and small cell lung cancer cells.

### Example 9: Experiment Comparing Sensitivity Depending on the Presence/Absence of SMARCA2/A4

Cytotoxic activity of a CBP/P300 inhibitor on SMARCA2/A4 deficient cells, i.e., A427 cells (pulmonary adenocarcinoma derived), H23 cells (pulmonary adenocarcinoma derived), COV434 cells (ovarian granulosa tumor derived), TOV112D cells (small cell ovarian cancer derived), SW13 cells (adrenocortical cancer derived) and DMS114 cells (small cell lung cancer derived), and SMARCA2/A4 wild-type cells, i.e., H1048 cells, H460 cells, 786-O cells, H2228 cells, H2009 cells, and H358 cells was compared.

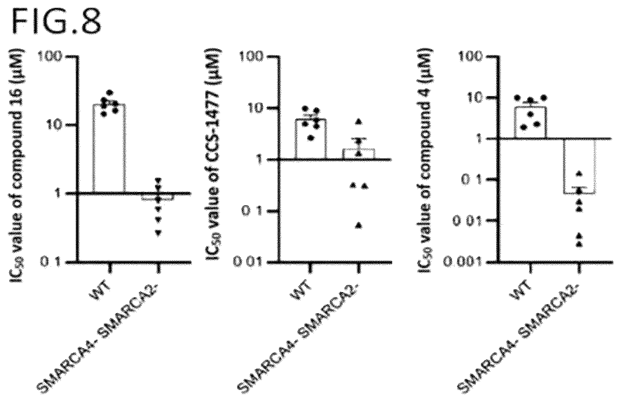

A427 cells, H23 cells, COV434 cells, TOV112D cells, SW13 cells, and DMS114 cells, and H1048 cells, H460 cells, 786-O cells, H2228 cells, H2009 cells, and H358 cells were seeded on a 96-well plate at 250 to 500 cells per well. After one day from seeding, compounds 4 and 16 or compound CCS-1477, which is known as a BRD inhibitor, were added so that the final concentration of DMSO would be 0.1%, and the cells were cultured for 6 days. After completion of culture, the cell survival rate was measured using CellTiter-Glo Luminescent Cell Viability Assay (Promega, G7570). The IC₅₀ value corresponding to the concentration of evaluated compounds at which a cell growth suppression ratio of 50% is exhibited was calculated from a cell survival curve. The IC₅₀ value of the SMARCA2/A4 deficient cell was compared with the IC₅₀ value of the SMARCA2/A4 wild-type cell group (FIG. 8).

As shown in FIG. 8, the IC₅₀ value of compounds 4 and 16 and CCS-1477 for the SMARCA2/A4 deficient cell group was less than the IC₅₀ value for the SMARCA2/A4 wild-type cell group. This demonstrated that a CBP/P300 inhibitor has a selective cell growth suppression effect on SMARCA2/A4 deficient pulmonary adenocarcinoma cells, ovarian granulosa tumor cells, small cell ovarian cancer cells, adrenocortical cancer cells, and small cell lung cancer cells.

### Example 10: Experiment on Suppression of Expression by siRNAR

SMARCA2/A4 deficient cells, i.e., H23 cells (pulmonary adenocarcinoma derived) and DMS114 cells (small cell lung cancer derived) and SMARCA2/A4 wild-type cells, i.e., H460 cells, were transfected with siRNA of a gene CREBBP encoding CBP (Dharmacon, L-003477-00-0005, hereinafter siCREBBP) and/or siRNA of gene EP300 encoding P300 (Dharmacon, L-003486-00-0005, hereinafter siEP300) to suppress expression of CREBBP and/or EP300.

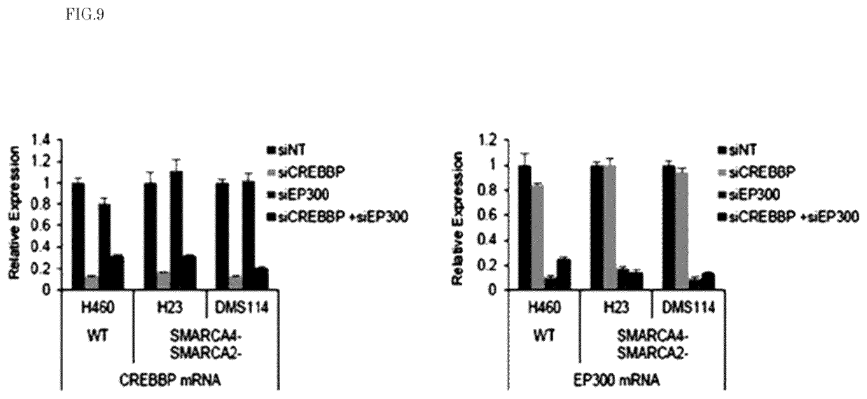

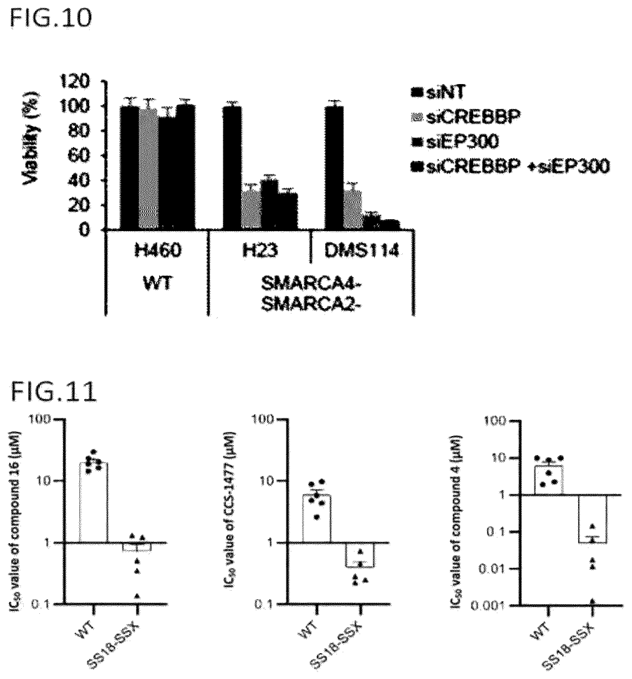

100000 cells were seeded on a 24-well plate per well, and 50 nM of siRNA and Lipofectamine RNAiMAX Transfection Reagent (ThermoFisher, 13778030) were added. After one day, the culture medium was exchanged. After another day, the cells were subcultured, and 50 nM of siRNA and Lipofectamine RNAiMAX Transfection Reagent were added again. After another day, the culture medium was exchanged. RNA was extracted from the cells. cDNA was prepared using a reverse transcriptase. The amount of expression of CREBBP and/or EP300 was checked by quantitative PCR (FIG. 9).

<Measurement of cell growth capability> A day after exchanging the culture medium, 250 to 500 cells were seeded on a 96-well plate per well, and the cells were cultured for 7 days. After completion of culture, the cell survival rate was measured using CellTiter-Glo Luminescent Cell Viability Assay (Promega, G7570) (FIG. 10).

As shown in FIG. 9, it was confirmed that the amount of mRNA of CREBBP and/or EP300 was reduced and expression was suppressed by treating each cell with siEP300 and/or siCREBBP. Significant decrease in cell survival rate (FIG. 10) was observed by suppressing the expression of CREBBP and/or EP300 in SMARCA2/4 deficient cells, i.e., H23 cells and DMS114 cells. This demonstrated that suppression of expression of CBP/P300 has a selective cell growth suppression effect on SMARCA2/A4 deficient pulmonary adenocarcinoma cells and small cell lung cancer cells.

### Example 11: Cell Growth Suppression Experiment

Cytotoxic activity of a CBP/P300 inhibitor on SMARCA4 deficient cells, i.e., H1693 cells (pulmonary adenocarcinoma derived) and H1819 cells (pulmonary adenocarcinoma derived), and SMARCA4 wild-type cells, i.e., H647 cells.

H1693 cells, H1819 cells, and H647 cells were obtained from the ATCC. The H1693 cells and H647 cells were cultured under 5% CO 2 conditions at 37° C. in a 10% fetal bovine serum and 1% penicillin/streptomycin-containing RPMI1640 medium. H1819 cells were cultured under 5% CO₂ conditions at 37° C. in a 5% fetal bovine serum and 1% penicillin/streptomycin-containing RPMI1640 medium.

A 384-well plate was seeded with 500 cells per well. After one day from seeding, compound 4 (Table 47) was added so that the final concentration of DMSO would be 0.14, and the cells were cultured for 3 days. After completion of culture, the cell survival rate was measured using CellTiter-Glo Luminescent Cell Viability Assay (Promega, G7570). The IC₅₀ value corresponding to the concentration of evaluated compounds at which a cell growth suppression ratio of 501 is exhibited was calculated from a cell survival curve. The results are shown in Table 53. Tests in which the cell survival rate was higher than 50% at 10 μM that is the maximum treatment concentration of each compound are indicated with an IC₅₀ value of “>10 μM”, and the cell survival rate (%) at treatment with 10 μM is indicated within the parenthesis.

TABLE 53

| | IC₅₀ (μM) | | |
|---|---|---|---|
| Compound | H1693 | H1819 | H647 |
| 4 | 0.1285 | 5.334 | >10 (60%) |

As shown in Table 53, CBP/P300 inhibitor, i.e., compound 4, exhibited a selective and potent cell growth suppression effect on SMARCA4 deficient cells, i.e., H1693 cells and H1819 cells. Meanwhile, hardly any cell growth suppression effect was exhibited on SMARCA4 expressing H647 cells.

This demonstrated that a CBP/P300 inhibitor has a selective and potent cell growth suppression effect on SMARCA4 deficient pulmonary adenocarcinama cells.

### 0.30 Example 12: Experiment Comparing Sensitivity Depending on the Presence/Absence of SS18-SSX Fusion Gene

Cytotoxic activity of a CRP/P300 inhibitor on SS18-SSX fusion cancer cells, i.e., Aska-SS cells (synovial sarcoma derived), Fuji cells (synovial sarcoma derived), YaFuss cells (synovial sarcoma derived), HS-SY-II cells (synovial sarcoma derived), and Yamato-SS cells (synovial sarcoma derived), and SS18/SSX wild-type cells, i.e., H1048 cells, H460 cells, 786-O cells, H2228 cells, H2009 cells, and H358 cells, was compared.

The Aska-SS cells, Fuji cells, YaFuss cells, HS-SY-II cells, and Yamato-SS cells, and H1048 cells, H460 cells, 786-O cells, H2228 cells, H2009 cells, and H358 cells were seeded on a 96-well plate at 250 to 500 cells per well. After one day from seeding, compounds 4 and 16 or compound CCS-1477, which is known as a BRD inhibitor, were added so that the final concentration of DMSO would be 0.1%, and the cells were cultured for 6 days. After completion of culture, the cell survival rate was measured using CellTiter-Glo Luminescent Cell Viability Assay (Promega, G7570). The IC₅₀ value corresponding to the concentration of evaluated compounds at which a cell growth suppression ratio of 50% is exhibited was calculated from a cell survival curve. The IC₅₀ value of the SS18/SSX wild-type cell group was compared with the IC₅₀ value of the SS18-SSX fusion cancer cell group (FIG. 11).

As shown in FIG. 11, the IC₅₀ value of compounds 4 and 16 and CCS-1477 for the SS18-SSX fusion cancer cell group was less than the IC₅₀ value for the SS18/SSX wild-type cell group. This demonstrated that a CBP/P300 inhibitor has a selective cell growth suppression effect on SS18-SSX fusion cancer, i.e., synovial sarcoma cells.

### Example 13: Experiment Comparing Sensitivity Depending on the Presence/Absence of ARID1

Cytotoxic activity of a CBP/P300 inhibitor on ARID1 deficient cancer cells, i.e., A2780 cells (ovarian cancer derived), RMG-V cells (ovarian cancer derived), TOV21G cells (ovarian cancer derived), and OVISE cells (ovarian cancer derived), and ARID1 wild-type cells, i.e., H1048 cells, H460 cells, 786-O cells, H2228 cells, H2009 cells, and H358 cells, was compared.

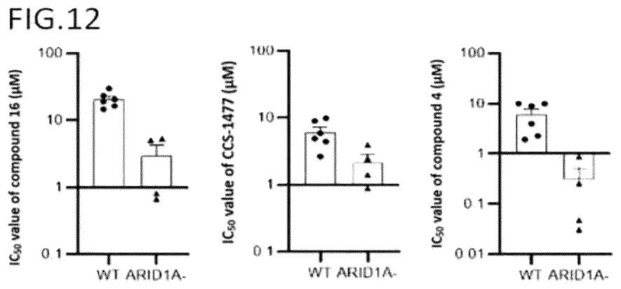

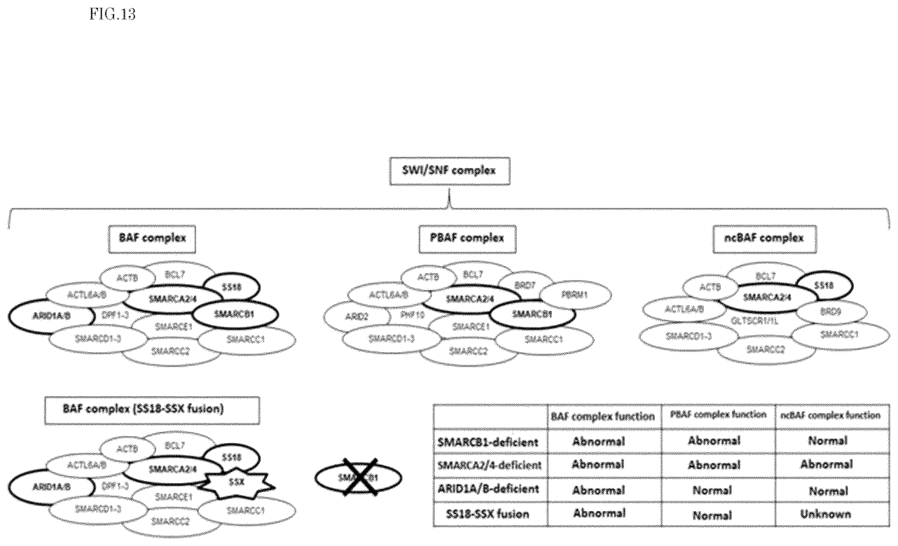

A2780 cells, RMG-V cells, TOV21G cells, and OVISE cells, and H1048 cells, H460 cells, 786-O cells, H2228 cells, H2009 cells, and H358 cells were seeded on a 96-well plate at 250 to 500 cells per well. After one day from seeding, compounds 4 and 16 or compound CCS-1477, which is known as a BRD inhibitor, were added so that the final concentration of DMSO would be 0.1%, and the cells were cultured for 6 days. After completion of culture, the cell survival rate was measured using CellTiter-Glo Luminescent Cell Viability Assay (Promega, G7570). The IC₅₀ value corresponding to the concentration of evaluated compounds at which a cell growth suppression ratio of 50% is exhibited was calculated from a cell survival curve. The IC₅₀ value of the ARID1 deficient cancer cell group was compared with the IC₅₀ value of the ARID1 wild-type cell group (FIG. 12).

As shown in FIG. 12, the IC₅₀ value of compounds 4 and 16 and CCS-1477 for the ARID1 deficient cancer cell group was less than the IC₅₀ value for the ARID1 wild-type cell group. This demonstrated that a CBP/P300 inhibitor has a selective cell growth suppression effect on ARID1 deficient cancer, i.e., ovarian cancer cells.

As described above, the present invention is exemplified by the use of its preferred embodiments. It is understood that the scope of the present invention should be interpreted solely based on the claims. The present application claims priority to Japanese Patent Application No. 2020-217707 (filed on Dec. 25, 2020) and Japanese Patent Application No. 2021-177849 (filed on Oct. 29, 2021) in Japan. The entire content thereof is incorporated herein by reference. It is understood that any patent, any patent application, and any references cited herein should be incorporated herein by reference in the same manner as the contents are specifically described herein.

1. A pharmaceutical composition for use in treating and/or preventing cancer, comprising a CBP/P300 inhibitor.

2. The pharmaceutical composition of claim 1, wherein the cancer is SWI/SNF complex dysfunction cancer.

3. The pharmaceutical composition of claim 2, wherein the SWI/SNF complex dysfunction cancer is BAF complex dysfunction cancer.

4. The pharmaceutical composition of claim 3, wherein the BAF complex dysfunction cancer comprises at least one selected from the group consisting of SMARC deficient cancer, SS18-SSX fusion cancer, and ARID deficient cancer.

5. The pharmaceutical composition of claim 1, wherein the cancer is SMARC deficient cancer.

6. The pharmaceutical composition of claim 5, wherein the SMARC deficient cancer comprises at least one selected from the group consisting of SMARCB1 deficient cancer, SMARCA2 deficient cancer, SMARCA4 deficient cancer, and SMARCA2/A4 deficient cancer.

7. The pharmaceutical composition of claim 5, wherein the SMARC deficient cancer is SMARCB1 deficient cancer.

8. The pharmaceutical composition of claim 7, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

9. The pharmaceutical composition of claim 7, wherein the SMARCB1 deficient cancer comprises at least one selected from the group consisting of malignant rhabdoid tumor, epithelioid sarcoma, and atypical teratoid/rhabdoid tumor.

10. The pharmaceutical composition of claim 7, wherein the SMARCB1 deficient cancer is malignant rhabdoid tumor.

11. The pharmaceutical composition of claim 5, wherein the SMARC deficient cancer is SMARCA2/A4 deficient cancer.

12. The pharmaceutical composition of claim 11, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

13. The pharmaceutical composition of claim 11, wherein the SMARCA2/A4 deficient cancer is pulmonary adenocarcinoma.

14. The pharmaceutical composition of claim 1, wherein the cancer is ARID deficient cancer.

15. The pharmaceutical composition of claim 14, wherein the ARID deficient cancer comprises at least one selected from the group consisting of ARID1A deficient cancer, ARID1B deficient cancer, and ARID1A/1B deficient cancer.

16. The pharmaceutical composition of claim 14, wherein the ARID deficient cancer is ARID1A deficient cancer.

17. The pharmaceutical composition of claim 16, wherein the ARID1A deficient cancer comprises at least one selected from the group consisting of ovarian cancer, gastric cancer, bile duct cancer, pancreatic cancer, uterine cancer, neuroblastoma, colon cancer, and bladder cancer.

18. The pharmaceutical composition of claim 16, wherein the ARID1A deficient cancer is ovarian cancer.

19. The pharmaceutical composition of claim 14, wherein the ARID deficient cancer is ARID1A/1B deficient cancer.

20. The pharmaceutical composition of claim 19, wherein the ARID1A/1B deficient cancer comprises at least one selected from the group consisting of ovarian cancer, colon cancer, uterine cancer, neuroblastoma, bladder cancer, and gastric cancer.

21. The pharmaceutical composition of claim 19, wherein the ARID1A/1B deficient cancer is ovarian cancer.

22. The pharmaceutical composition of claim 1, wherein the cancer is SS18-SSX fusion cancer.

23. The pharmaceutical composition of claim 22, wherein the SS18-SSX fusion cancer is synovial sarcoma or Ewing's sarcoma.

24. The pharmaceutical composition of claim 22, wherein the SS18-SSX fusion cancer is synovial sarcoma.

25. The pharmaceutical composition of any one of claims 1 to 24, wherein the CBP/P300 inhibitor is a HAT inhibitor, a BRD inhibitor, an antisense nucleic acid for a transcriptional product of a gene encoding CBP or P300, a ribozyme for a transcriptional product of a gene encoding CBP or P300, or a nucleic acid having RNAi activity for a transcriptional product of a gene encoding CBP or P300, or a precursor thereof.

26. The pharmaceutical composition of claim 25, wherein the CBP/P300 inhibitor is a HAT inhibitor or a BRD inhibitor.

27. The pharmaceutical composition of claim 26, wherein the CBP/P300 inhibitor is a HAT inhibitor.

28. The pharmaceutical composition of any one of claims 25 to 27, wherein activity of the HAT inhibitor inhibits histone acetyltransferase (HAT) activity of CBP and/or P300 by 50% or more at 20 μM.

29. The pharmaceutical composition of any one of claims 25 to 27, wherein activity of the HAT inhibitor inhibits histone acetyltransferase (HAT) activity of CBP and/or P300 by 80% or more at 20 μM.

30. The pharmaceutical composition of any one of claims 1 to 29, wherein the CBP/P300 inhibitor is a nucleic acid or a low molecular weight compound.

31. The pharmaceutical composition of any one of claims 25 to 30, wherein the HAT inhibitor is a low molecular weight compound.

32. The pharmaceutical composition of claim 31, wherein the low molecular weight compound is a compound represented by formula (1) 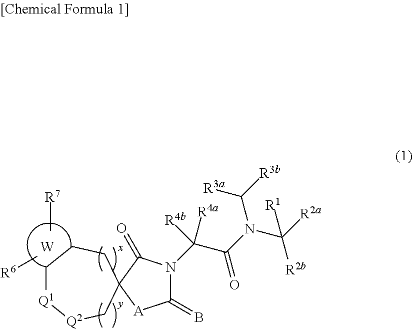
wherein
Q¹ - - - Q² is —C(R¹⁰)₂—C(R¹⁴)₂—, —O—C(R¹⁴)₂—, —O—C(O)—, —S(O)₂—C(R¹⁴)₂—, —S—C(R¹⁴)₂—, —NR⁹—C(O)—, —NR⁹—C(R¹⁴)₂—, —C(R¹⁰)₂—O—, —C(R¹⁰)₂—, or C(R¹⁰)═C(R¹⁴)—;
A is —NR⁸—, —O—, or —S—;
B is O or NH;
W is arylene or heteroarylene;
R¹ is carbocyclyl or heterocyclyl;
R^{2a} and R^{2b} are each independently a hydrogen atom, a deuterium atom, C₁₋₆ alkyl, C₁₋₆ alkenyl, or C₁₋₆ alkynyl;
R^{3a} is a hydrogen atom, C(O)NH₂, C₁₋₆ alkyl, C₁₋₆ alkenyl, C₁₋₆ alkynyl, aryl, cycloalkyl, or heterocyclyl;
R^{3b} is C₁₋₆ alkyl, C₁₋₆ alkenyl, C₁₋₆ alkynyl, aryl, cycloalkyl, or heterocyclyl; or
wherein R^{3a} and R^{3b}, together with the carbon atom to which they are attached, may form arene, cycloalkane, or heterocyclyl;
R^{4a} and R^{4b} are each independently a hydrogen atom, a deuterium atom, C₁₋₆ alkyl, C₁₋₆ alkenyl, or C₁₋₆ alkynyl;
R⁶ and R⁷ are each independently a hydrogen atom, a halogen atom, —OH, —CN, —CO₂H, C₁₋₆ alkyl, C₁₋₆ alkenyl, C₁₋₆ alkynyl, alkoxy, haloalkoxy, alkoxyalkyl, haloalkoxyalkyl, hydroxyalkyl, hydroxyalkynyl, aryl, cycloalkyl, heterocyclyl, heterocyclylalkyl, heterocyclyloxy, —B(R¹¹)(R¹³), —S(O)ₘR¹², —N(R¹²)₂, —C(═O)N(R¹²)₂, —NHC(═O)R¹², —NHC(═O)OR¹², —NHC(═O)C(═O)N(R¹²)₂, —NHC(═O)C(═O)OR¹², —NHC(═O)N(R¹²)₂, —NHC(═O)NR¹²C(═O)N(R¹²)₂, NHC(═O)NR¹²S(O)₂OR¹², —NHC(═O)NR¹²S(O)₂N(R¹²)₂, —NHC(═S) N(R¹²)₂, —NHC(═N—C≡N) NR¹², —NHC(═N—C≡N) SR¹², or —NHS(O)ₘR¹²;
R⁸ and R⁹ are each independently a hydrogen atom, C₁₋₆ alkyl, C₁₋₆ alkenyl, or C₁₋₆ alkynyl;
R¹⁰ is, for each instance, each independently a hydrogen atom, —OH, a halogen atom, —CN, —C₀₂R¹², —C(═O)NHR¹³, —NHR¹², C₁₋₆ alkyl, C₁₋₆ alkenyl, C₁₋₆ alkynyl, or alkoxy; or wherein two R¹⁰ together may form oxo or ═N—OR¹¹;
R¹¹ and R¹³ are each independently a hydrogen atom, —OH, C₁₋₆ alkyl, C₁₋₆ alkenyl, or C₁₋₆ alkynyl;
R¹² is, for each instance, each independently a hydrogen atom, C₁₋₆ alkyl, C₁₋₆ alkenyl, C₁₋₆ alkynyl, aryl, cycloalkyl, or heterocyclyl;
R¹⁴ is, for each instance, each independently a hydrogen atom, C₁₋₆ alkyl, C₁₋₆ alkenyl, or C₁₋₆ alkynyl;
m is, for each instance, each independently 0, 1, or 2;
x and y are each independently 0 or 1, wherein x and y are chosen so that the sum of x+y is 0 or 1;
with the proviso that if R¹ and W are each unsubstituted phenyl, A is —NH, x is 0 or 1, y is 0, and Q¹ - - - Q² is —C(R¹⁰)₂—C(R¹⁴)₂—, each of R^{3a} and R^{3b} is not cyclopropyl or methyl; and
if at least one of R¹ and W is unsubstituted phenyl, and A is —NH, R^{3a} and R^{3b}, together with the carbon atom to which they are attached, do not form tetrahydrothiophene 1,1-dioxide or tetrahydrothiophene.

33. The pharmaceutical composition of claim 31, wherein the low molecular weight compound is compound represented by the following (Table 1)
TABLE 1 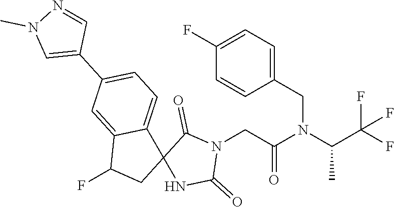 [SMILES: C[C@H](N(Cc1ccc(F)cc1)C(=O)CN1C(=O)NC2(CC(F)c3cc(-c4cnn(C)c4)ccc32)C1=O)C(F)(F)F] 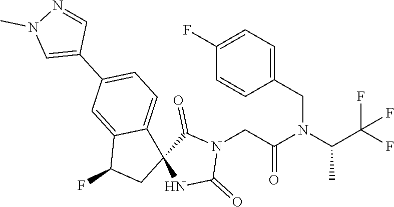 [SMILES: C[C@H](N(Cc1ccc(F)cc1)C(=O)CN1C(=O)N[C@]2(C[C@@H](F)c3cc(-c4cnn(C)c4)ccc32)C1=O)C(F)(F)F] 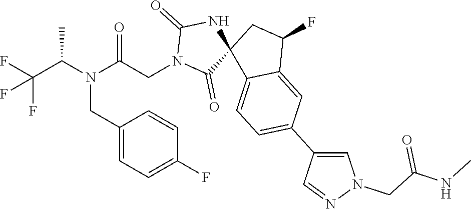 [SMILES: CNC(=O)Cn1cc(-c2ccc3c(c2)[C@H](F)C[C@]32NC(=O)N(CC(=O)N(Cc3ccc(F)cc3)[C@@H](C)C(F)(F)F)C2=O)cn1] 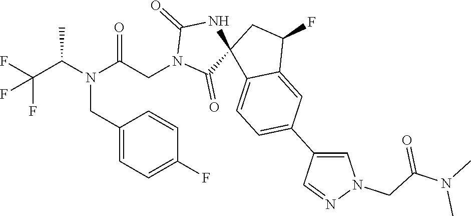 [SMILES: C[C@H](N(Cc1ccc(F)cc1)C(=O)CN1C(=O)N[C@]2(C[C@@H](F)c3cc(-c4cnn(CC(=O)N(C)C)c4)ccc32)C1=O)C(F)(F)F] 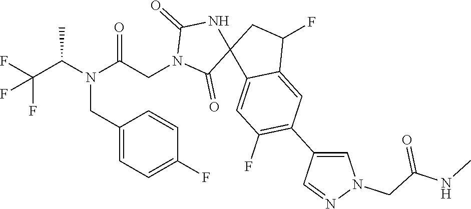 [SMILES: CNC(=O)Cn1cc(-c2cc3c(cc2F)C2(CC3F)NC(=O)N(CC(=O)N(Cc3ccc(F)cc3)[C@@H](C)C(F)(F)F)C2=O)cn1] 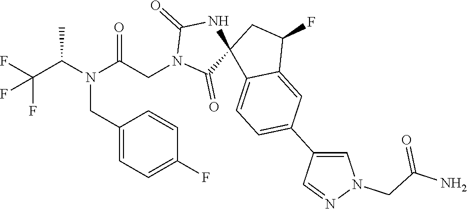 [SMILES: C[C@H](N(Cc1ccc(F)cc1)C(=O)CN1C(=O)N[C@]2(C[C@@H](F)c3cc(-c4cnn(CC(N)=O)c4)ccc32)C1=O)C(F)(F)F] 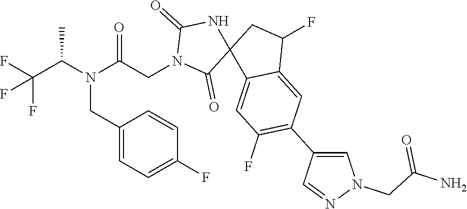 [SMILES: C[C@H](N(Cc1ccc(F)cc1)C(=O)CN1C(=O)NC2(CC(F)c3cc(-c4cnn(CC(N)=O)c4)c(F)cc32)C1=O)C(F)(F)F] 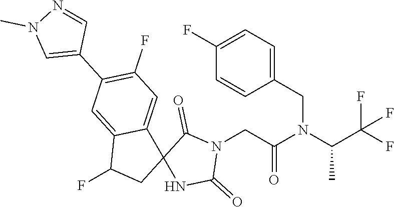 [SMILES: C[C@H](N(Cc1ccc(F)cc1)C(=O)CN1C(=O)NC2(CC(F)c3cc(-c4cnn(C)c4)c(F)cc32)C1=O)C(F)(F)F] 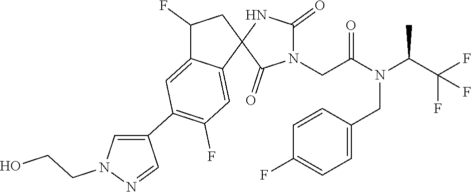 [SMILES: C[C@H](N(Cc1ccc(F)cc1)C(=O)CN1C(=O)NC2(CC(F)c3cc(-c4cnn(CCO)c4)c(F)cc32)C1=O)C(F)(F)F] 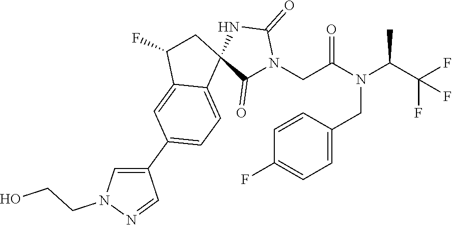 [SMILES: C[C@H](N(Cc1ccc(F)cc1)C(=O)CN1C(=O)N[C@]2(C[C@@H](F)c3cc(-c4cnn(CCO)c4)ccc32)C1=O)C(F)(F)F] 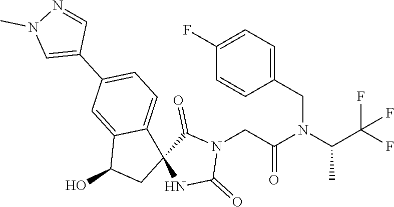 [SMILES: C[C@H](N(Cc1ccc(F)cc1)C(=O)CN1C(=O)N[C@]2(C[C@@H](O)c3cc(-c4cnn(C)c4)ccc32)C1=O)C(F)(F)F] 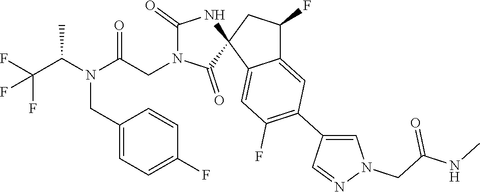 [SMILES: CNC(=O)Cn1cc(-c2cc3c(cc2F)[C@]2(C[C@H]3F)NC(=O)N(CC(=O)N(Cc3ccc(F)cc3)[C@@H](C)C(F)(F)F)C2=O)cn1] 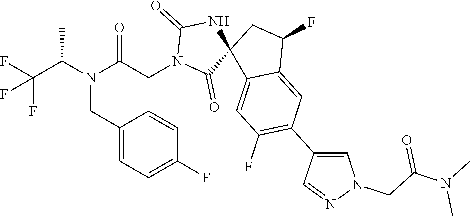 [SMILES: C[C@H](N(Cc1ccc(F)cc1)C(=O)CN1C(=O)N[C@]2(C[C@@H](F)c3cc(-c4cnn(CC(=O)N(C)C)c4)c(F)cc32)C1=O)C(F)(F)F] 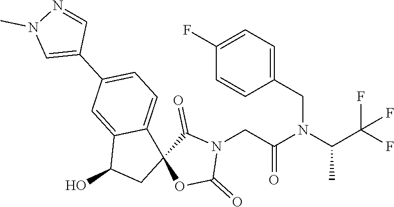 [SMILES: C[C@H](N(Cc1ccc(F)cc1)C(=O)CN1C(=O)O[C@]2(C[C@@H](O)c3cc(-c4cnn(C)c4)ccc32)C1=O)C(F)(F)F] 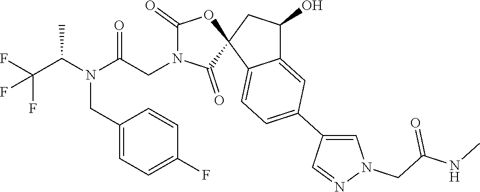 [SMILES: CNC(=O)Cn1cc(-c2ccc3c(c2)[C@H](O)C[C@]32OC(=O)N(CC(=O)N(Cc3ccc(F)cc3)[C@@H](C)C(F)(F)F)C2=O)cn1] 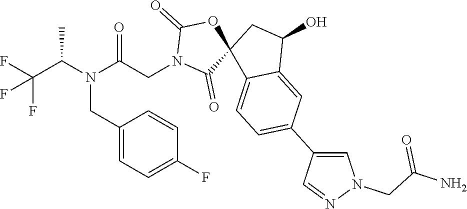 [SMILES: C[C@H](N(Cc1ccc(F)cc1)C(=O)CN1C(=O)O[C@]2(C[C@@H](O)c3cc(-c4cnn(CC(N)=O)c4)ccc32)C1=O)C(F)(F)F] 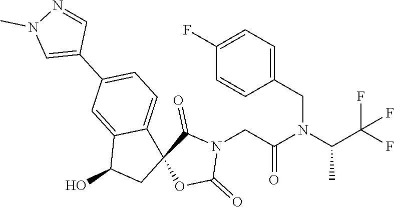 [SMILES: C[C@H](N(Cc1ccc(F)cc1)C(=O)CN1C(=O)O[C@@]2(C[C@@H](O)c3cc(-c4cnn(C)c4)ccc32)C1=O)C(F)(F)F] 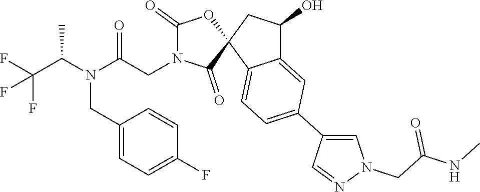 [SMILES: CNC(=O)Cn1cc(-c2ccc3c(c2)[C@H](O)C[C@@]32OC(=O)N(CC(=O)N(Cc3ccc(F)cc3)[C@@H](C)C(F)(F)F)C2=O)cn1] 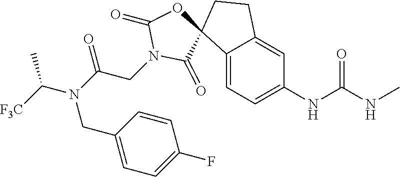 [SMILES: CNC(=O)Nc1ccc2c(c1)CC[C@]21OC(=O)N(CC(=O)N(Cc2ccc(F)cc2)[C@@H](C)C(F)(F)F)C1=O] 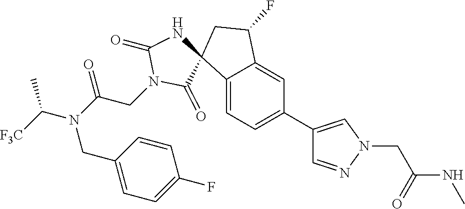 [SMILES: CNC(=O)Cn1cc(-c2ccc3c(c2)[C@@H](F)C[C@]32NC(=O)N(CC(=O)N(Cc3ccc(F)cc3)[C@@H](C)C(F)(F)F)C2=O)cn1] 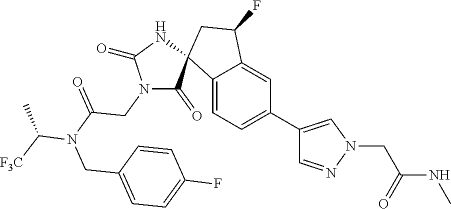 [SMILES: CNC(=O)Cn1cc(-c2ccc3c(c2)[C@H](F)C[C@@]32NC(=O)N(CC(=O)N(Cc3ccc(F)cc3)[C@@H](C)C(F)(F)F)C2=O)cn1] 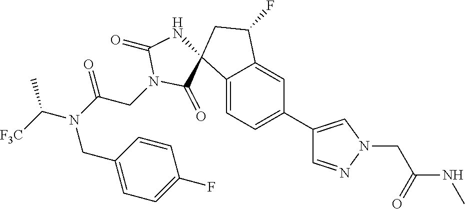 [SMILES: CNC(=O)Cn1cc(-c2ccc3c(c2)[C@@H](F)C[C@@]32NC(=O)N(CC(=O)N(Cc3ccc(F)cc3)[C@@H](C)C(F)(F)F)C2=O)cn1] 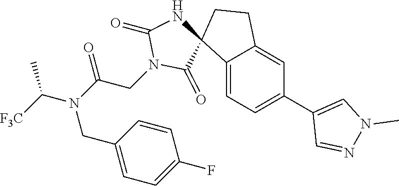 [SMILES: C[C@H](N(Cc1ccc(F)cc1)C(=O)CN1C(=O)N[C@]2(CCc3cc(-c4cnn(C)c4)ccc32)C1=O)C(F)(F)F]
or a pharmaceutically acceptable salt thereof.

34. The pharmaceutical composition of claim 31, wherein the low molecular weight compound is a compound represented by formula (2) 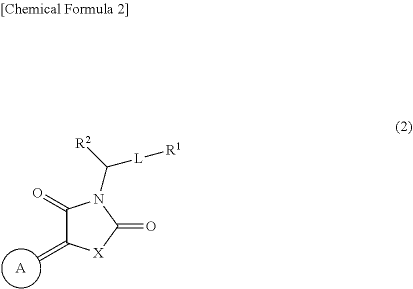
wherein
A is carbocyclyl or heterocyclyl with a 6-, 7-, or 8-membered ring, and heterocyclyl is comprised of a carbon atom, and one or more heteroatoms selected from O and S;
X is —S— or —NH—;
L is a direct bond or a linker;
R¹ is aryl, heteroaryl, or cycloalkyl;
R² is a hydrogen atom, a deuterium atom, C₁₋₆ alkyl, C₁₋₆ alkenyl, or C₁₋₆ alkynyl;
with the proviso that if A is unsubstituted cyclohexyl, R₂ is a hydrogen atom, and X is —S—, R¹ is not p-aminosulfonylphenyl or p-fluorophenyl,
or a prodrug thereof or a pharmaceutically acceptable salt thereof.

35. The pharmaceutical composition of claim 31, wherein the low molecular weight compound is a compound represented by the following (Table 2)
TABLE 2-1 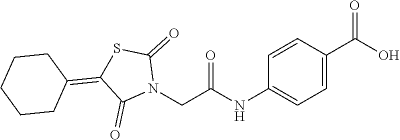 [SMILES: O=C(CN1C(=O)SC(=C2CCCCC2)C1=O)Nc1ccc(C(=O)O)cc1] 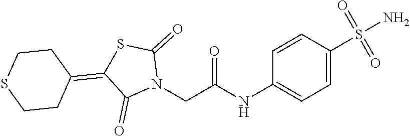 [SMILES: NS(=O)(=O)c1ccc(NC(=O)CN2C(=O)SC(=C3CCSCC3)C2=O)cc1] 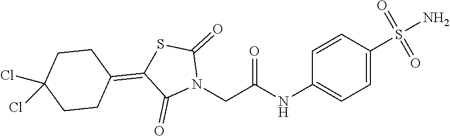 [SMILES: NS(=O)(=O)c1ccc(NC(=O)CN2C(=O)SC(=C3CCC(Cl)(Cl)CC3)C2=O)cc1] 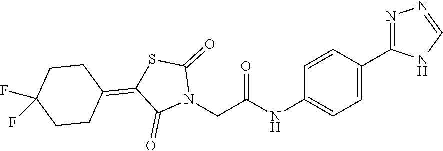 [SMILES: O=C(CN1C(=O)SC(=C2CCC(F)(F)CC2)C1=O)Nc1ccc(-c2nnc[nH]2)cc1] 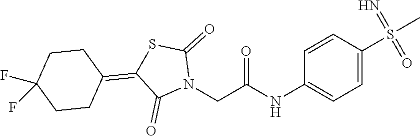 [SMILES: CS(=N)(=O)c1ccc(NC(=O)CN2C(=O)SC(=C3CCC(F)(F)CC3)C2=O)cc1] 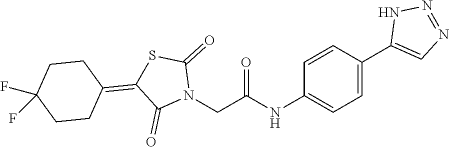 [SMILES: O=C(CN1C(=O)SC(=C2CCC(F)(F)CC2)C1=O)Nc1ccc(-c2cnn[nH]2)cc1] 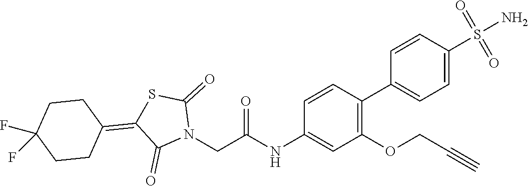 [SMILES: C#CCOc1cc(NC(=O)CN2C(=O)SC(=C3CCC(F)(F)CC3)C2=O)ccc1-c1ccc(S(N)(=O)=O)cc1] 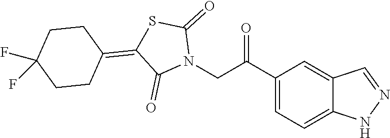 [SMILES: O=C(CN1C(=O)SC(=C2CCC(F)(F)CC2)C1=O)c1ccc2[nH]ncc2c1] 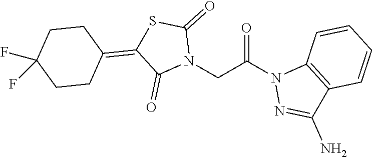 [SMILES: Nc1nn(C(=O)CN2C(=O)SC(=C3CCC(F)(F)CC3)C2=O)c2ccccc12] 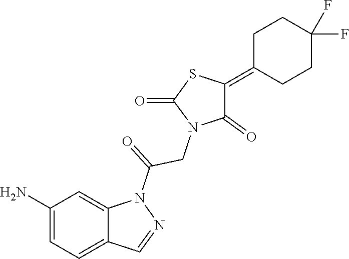 [SMILES: Nc1ccc2cnn(C(=O)CN3C(=O)SC(=C4CCC(F)(F)CC4)C3=O)c2c1] 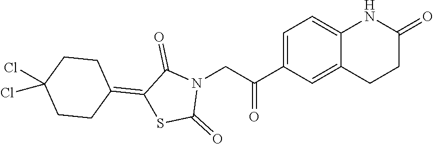 [SMILES: O=C1CCc2cc(C(=O)CN3C(=O)SC(=C4CCC(Cl)(Cl)CC4)C3=O)ccc2N1] 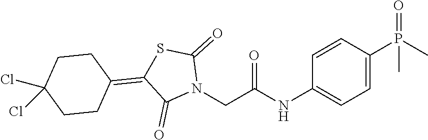 [SMILES: CP(C)(=O)c1ccc(NC(=O)CN2C(=O)SC(=C3CCC(Cl)(Cl)CC3)C2=O)cc1] 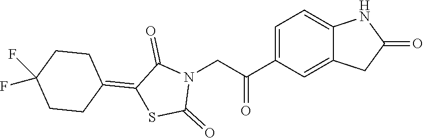 [SMILES: O=C1Cc2cc(C(=O)CN3C(=O)SC(=C4CCC(F)(F)CC4)C3=O)ccc2N1] 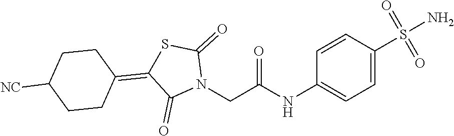 [SMILES: N#CC1CCC(=C2SC(=O)N(CC(=O)Nc3ccc(S(N)(=O)=O)cc3)C2=O)CC1] 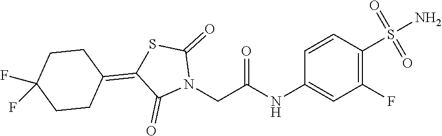 [SMILES: NS(=O)(=O)c1ccc(NC(=O)CN2C(=O)SC(=C3CCC(F)(F)CC3)C2=O)cc1F] 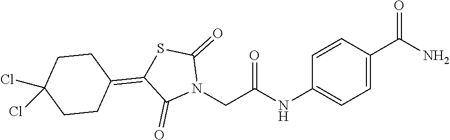 [SMILES: NC(=O)c1ccc(NC(=O)CN2C(=O)SC(=C3CCC(Cl)(Cl)CC3)C2=O)cc1] 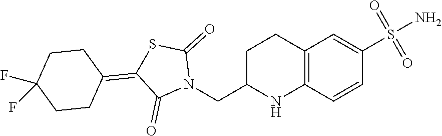 [SMILES: NS(=O)(=O)c1ccc2c(c1)CCC(CN1C(=O)SC(=C3CCC(F)(F)CC3)C1=O)N2] 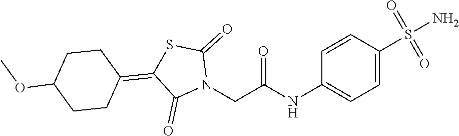 [SMILES: COC1CCC(=C2SC(=O)N(CC(=O)Nc3ccc(S(N)(=O)=O)cc3)C2=O)CC1] 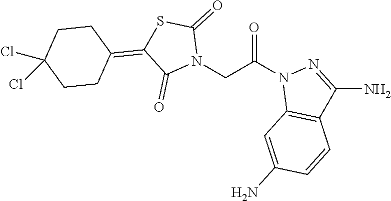 [SMILES: Nc1ccc2c(N)nn(C(=O)CN3C(=O)SC(=C4CCC(Cl)(Cl)CC4)C3=O)c2c1] 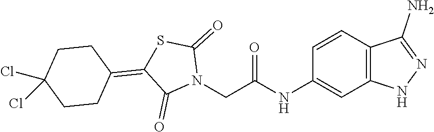 [SMILES: Nc1n[nH]c2cc(NC(=O)CN3C(=O)SC(=C4CCC(Cl)(Cl)CC4)C3=O)ccc12] 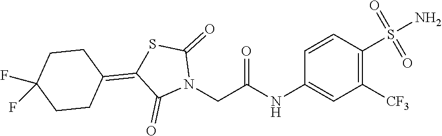 [SMILES: NS(=O)(=O)c1ccc(NC(=O)CN2C(=O)SC(=C3CCC(F)(F)CC3)C2=O)cc1C(F)(F)F] 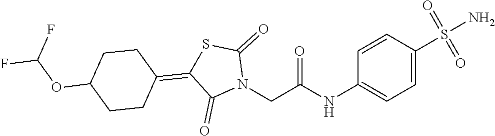 [SMILES: NS(=O)(=O)c1ccc(NC(=O)CN2C(=O)SC(=C3CCC(OC(F)F)CC3)C2=O)cc1] 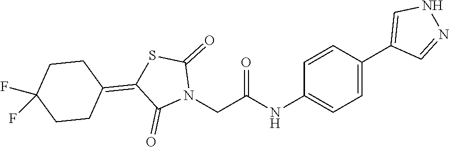 [SMILES: O=C(CN1C(=O)SC(=C2CCC(F)(F)CC2)C1=O)Nc1ccc(-c2cn[nH]c2)cc1] 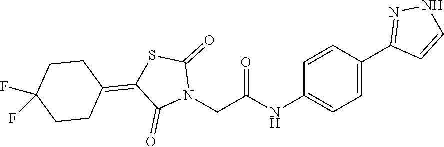 [SMILES: O=C(CN1C(=O)SC(=C2CCC(F)(F)CC2)C1=O)Nc1ccc(-c2cc[nH]n2)cc1] 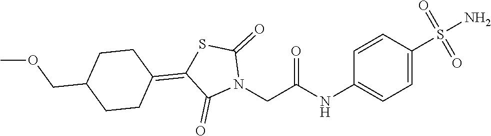 [SMILES: COCC1CCC(=C2SC(=O)N(CC(=O)Nc3ccc(S(N)(=O)=O)cc3)C2=O)CC1] 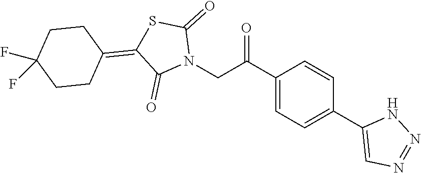 [SMILES: O=C(CN1C(=O)SC(=C2CCC(F)(F)CC2)C1=O)c1ccc(-c2cnn[nH]2)cc1] 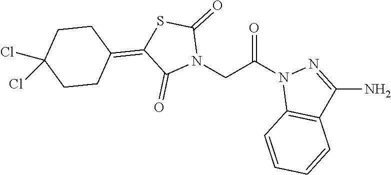 [SMILES: Nc1nn(C(=O)CN2C(=O)SC(=C3CCC(Cl)(Cl)CC3)C2=O)c2ccccc12] 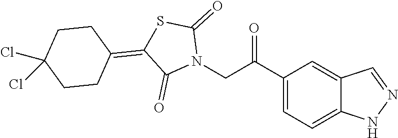 [SMILES: O=C(CN1C(=O)SC(=C2CCC(Cl)(Cl)CC2)C1=O)c1ccc2[nH]ncc2c1] 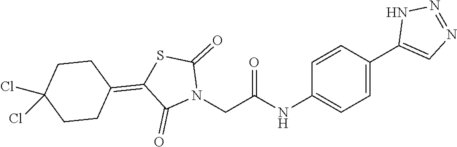 [SMILES: O=C(CN1C(=O)SC(=C2CCC(Cl)(Cl)CC2)C1=O)Nc1ccc(-c2cnn[nH]2)cc1] 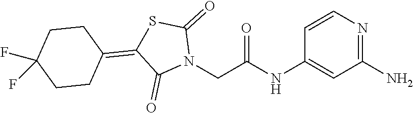 [SMILES: Nc1cc(NC(=O)CN2C(=O)SC(=C3CCC(F)(F)CC3)C2=O)ccn1] 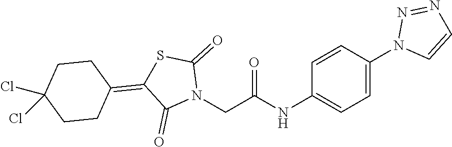 [SMILES: O=C(CN1C(=O)SC(=C2CCC(Cl)(Cl)CC2)C1=O)Nc1ccc(-n2ccnn2)cc1] 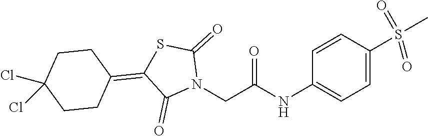 [SMILES: CS(=O)(=O)c1ccc(NC(=O)CN2C(=O)SC(=C3CCC(Cl)(Cl)CC3)C2=O)cc1] 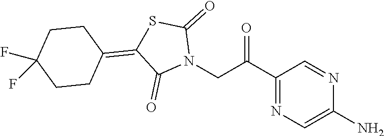 [SMILES: Nc1cnc(C(=O)CN2C(=O)SC(=C3CCC(F)(F)CC3)C2=O)cn1] 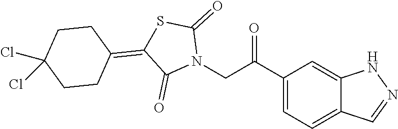 [SMILES: O=C(CN1C(=O)SC(=C2CCC(Cl)(Cl)CC2)C1=O)c1ccc2cn[nH]c2c1] 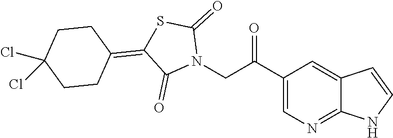 [SMILES: O=C(CN1C(=O)SC(=C2CCC(Cl)(Cl)CC2)C1=O)c1cnc2[nH]ccc2c1] 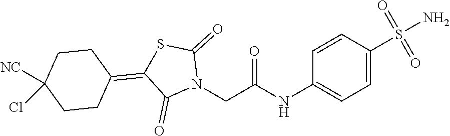 [SMILES: N#CC1(Cl)CCC(=C2SC(=O)N(CC(=O)Nc3ccc(S(N)(=O)=O)cc3)C2=O)CC1] 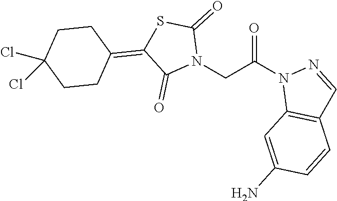 [SMILES: Nc1ccc2cnn(C(=O)CN3C(=O)SC(=C4CCC(Cl)(Cl)CC4)C3=O)c2c1] 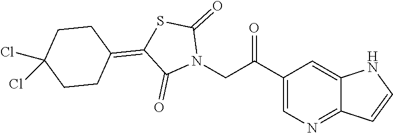 [SMILES: O=C(CN1C(=O)SC(=C2CCC(Cl)(Cl)CC2)C1=O)c1cnc2cc[nH]c2c1] 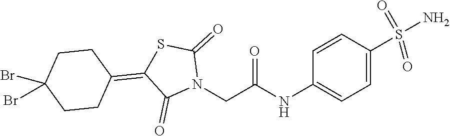 [SMILES: NS(=O)(=O)c1ccc(NC(=O)CN2C(=O)SC(=C3CCC(Br)(Br)CC3)C2=O)cc1]
TABLE 2-2 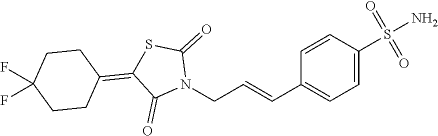 [SMILES: NS(=O)(=O)c1ccc(/C=C/CN2C(=O)SC(=C3CCC(F)(F)CC3)C2=O)cc1] 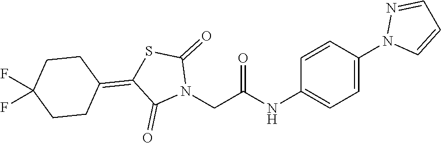 [SMILES: O=C(CN1C(=O)SC(=C2CCC(F)(F)CC2)C1=O)Nc1ccc(-n2cccn2)cc1] 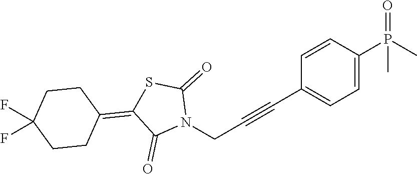 [SMILES: CP(C)(=O)c1ccc(C#CCN2C(=O)SC(=C3CCC(F)(F)CC3)C2=O)cc1] 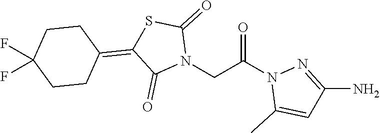 [SMILES: Cc1cc(N)nn1C(=O)CN1C(=O)SC(=C2CCC(F)(F)CC2)C1=O] 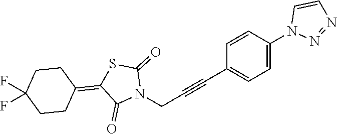 [SMILES: O=C1SC(=C2CCC(F)(F)CC2)C(=O)N1CC#Cc1ccc(-n2ccnn2)cc1] 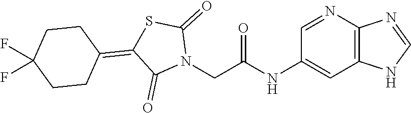 [SMILES: O=C(CN1C(=O)SC(=C2CCC(F)(F)CC2)C1=O)Nc1cnc2nc[nH]c2c1] 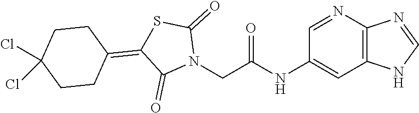 [SMILES: O=C(CN1C(=O)SC(=C2CCC(Cl)(Cl)CC2)C1=O)Nc1cnc2nc[nH]c2c1] 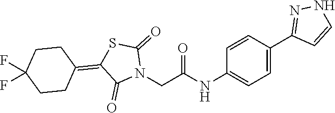 [SMILES: O=C(CN1C(=O)SC(=C2CCC(F)(F)CC2)C1=O)Nc1ccc(-c2cc[nH]n2)cc1] 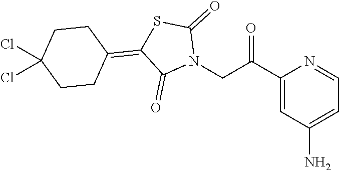 [SMILES: Nc1ccnc(C(=O)CN2C(=O)SC(=C3CCC(Cl)(Cl)CC3)C2=O)c1] 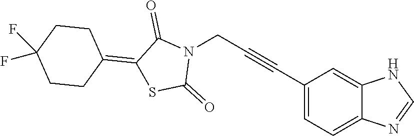 [SMILES: O=C1SC(=C2CCC(F)(F)CC2)C(=O)N1CC#Cc1ccc2nc[nH]c2c1] 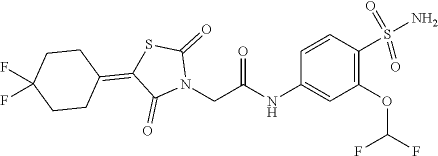 [SMILES: NS(=O)(=O)c1ccc(NC(=O)CN2C(=O)SC(=C3CCC(F)(F)CC3)C2=O)cc1OC(F)F] 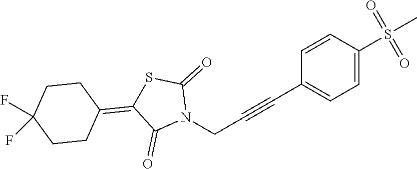 [SMILES: CS(=O)(=O)c1ccc(C#CCN2C(=O)SC(=C3CCC(F)(F)CC3)C2=O)cc1] 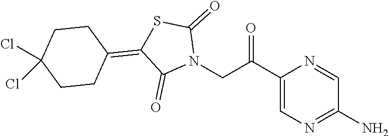 [SMILES: Nc1cnc(C(=O)CN2C(=O)SC(=C3CCC(Cl)(Cl)CC3)C2=O)cn1] 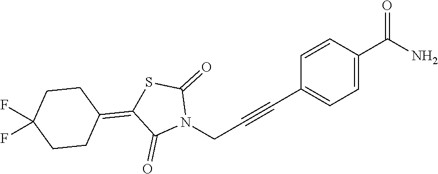 [SMILES: NC(=O)c1ccc(C#CCN2C(=O)SC(=C3CCC(F)(F)CC3)C2=O)cc1] 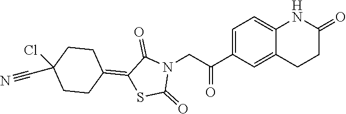 [SMILES: N#CC1(Cl)CCC(=C2SC(=O)N(CC(=O)c3ccc4c(c3)CCC(=O)N4)C2=O)CC1] 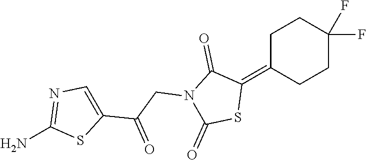 [SMILES: Nc1ncc(C(=O)CN2C(=O)SC(=C3CCC(F)(F)CC3)C2=O)s1] 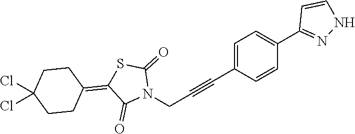 [SMILES: O=C1SC(=C2CCC(Cl)(Cl)CC2)C(=O)N1CC#Cc1ccc(-c2cc[nH]n2)cc1] 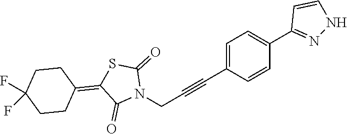 [SMILES: O=C1SC(=C2CCC(F)(F)CC2)C(=O)N1CC#Cc1ccc(-c2cc[nH]n2)cc1] 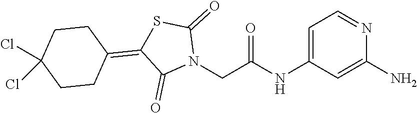 [SMILES: Nc1cc(NC(=O)CN2C(=O)SC(=C3CCC(Cl)(Cl)CC3)C2=O)ccn1] 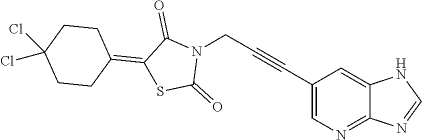 [SMILES: O=C1SC(=C2CCC(Cl)(Cl)CC2)C(=O)N1CC#Cc1cnc2nc[nH]c2c1] 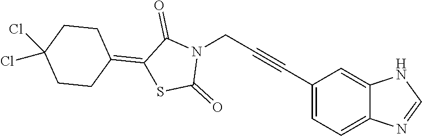 [SMILES: O=C1SC(=C2CCC(Cl)(Cl)CC2)C(=O)N1CC#Cc1ccc2nc[nH]c2c1] 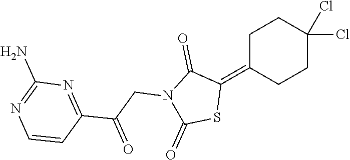 [SMILES: Nc1nccc(C(=O)CN2C(=O)SC(=C3CCC(Cl)(Cl)CC3)C2=O)n1] 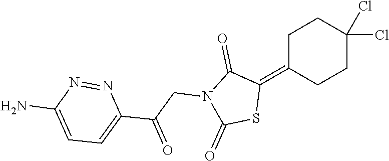 [SMILES: Nc1ccc(C(=O)CN2C(=O)SC(=C3CCC(Cl)(Cl)CC3)C2=O)nn1] 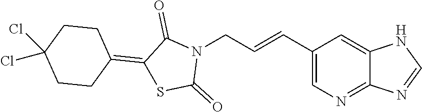 [SMILES: O=C1SC(=C2CCC(Cl)(Cl)CC2)C(=O)N1C/C=C/c1cnc2nc[nH]c2c1] 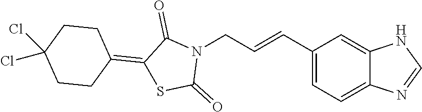 [SMILES: O=C1SC(=C2CCC(Cl)(Cl)CC2)C(=O)N1C/C=C/c1ccc2nc[nH]c2c1]
or a pharmaceutically acceptable salt thereof.

36. The pharmaceutical composition of claim 31, wherein the low molecular weight compound is a compound represented by formula (3) 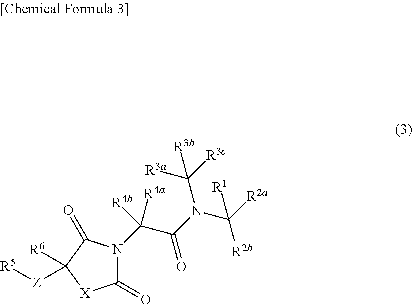
wherein
X is —NH— or O—;
Z is a direct bond or —C(R^{7a})(R^{7b})—;
R¹ is carbocyclyl or heterocyclyl;
R^{2a} and R^{2b} are each independently a hydrogen atom, a deuterium atom, C₁₋₆ alkyl, C₁₋₆ alkenyl, or C₁₋₆ alkynyl;
R^{3a} is carbocyclyl or heterocyclyl, and R^{3b} is C₁₋₆ alkyl, C₁₋₆ alkenyl, C₁₋₆ alkynyl, or carbocyclyl, or R^{3a} and R^{3b} are each independently C₁₋₆ alkyl, C₁₋₆ alkenyl, or C₁₋₆ alkynyl, wherein R^{3a} and R^{3b}, together with the carbon atom to which they are attached, may form carbocyclyl or heterocyclyl;
R^{3c} is a hydrogen atom or a deuterium atom;
R^{4a} and R^{4b} are each independently a hydrogen atom, a deuterium atom, C₁₋₆ alkyl, C₁₋₆ alkenyl, or C₁₋₆ alkynyl;
R⁵ is carbocyclyl or heterocyclyl;
R⁶ is, when Z is a direct bond, a hydrogen atom or a deuterium atom; or is, when Z is —C(R^{7a})(R^{7b})—, a hydrogen atom, a deuterium atom, C₁₋₆ alkyl, C₁₋₆ alkenyl, or C₁₋₆ alkynyl;
R^{7a} and R^{7b} are each independently a hydrogen atom, a deuterium atom, C₁₋₆ alkyl, C₁₋₆ alkenyl, or C₁₋₆ alkynyl, with the proviso that if Z is —CH₂—, R¹ is unsubstituted phenyl, and R⁵ is unsubstituted indolyl, each of R^{3a}, R^{3b}, and R^{3c} is not unsubstituted cyclopropyl, methyl, or a hydrogen atom,
or a prodrug thereof or a pharmaceutically acceptable salt thereof.

37. The pharmaceutical composition of claim 31, wherein the low molecular weight compound is a compound represented by the following (Table 3)
TABLE 3-1 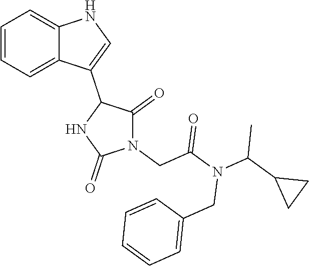 [SMILES: CC(C1CC1)N(Cc1ccccc1)C(=O)CN1C(=O)NC(c2c[nH]c3ccccc23)C1=O] 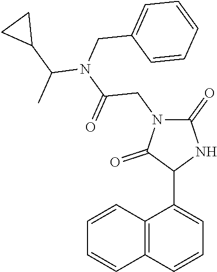 [SMILES: CC(C1CC1)N(Cc1ccccc1)C(=O)CN1C(=O)NC(c2cccc3ccccc23)C1=O] 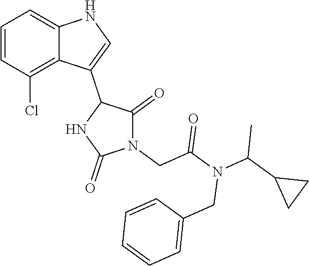 [SMILES: CC(C1CC1)N(Cc1ccccc1)C(=O)CN1C(=O)NC(c2c[nH]c3cccc(Cl)c23)C1=O] 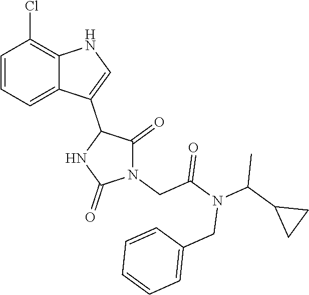 [SMILES: CC(C1CC1)N(Cc1ccccc1)C(=O)CN1C(=O)NC(c2c[nH]c3c(Cl)cccc23)C1=O] 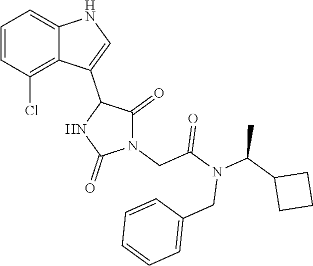 [SMILES: C[C@@H](C1CCC1)N(Cc1ccccc1)C(=O)CN1C(=O)NC(c2c[nH]c3cccc(Cl)c23)C1=O] 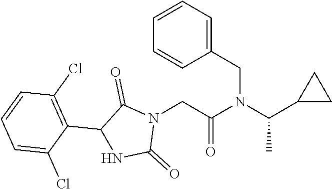 [SMILES: C[C@@H](C1CC1)N(Cc1ccccc1)C(=O)CN1C(=O)NC(c2c(Cl)cccc2Cl)C1=O] 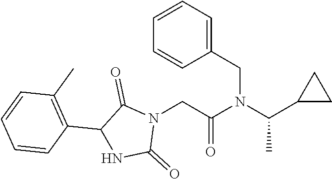 [SMILES: Cc1ccccc1C1NC(=O)N(CC(=O)N(Cc2ccccc2)[C@@H](C)C2CC2)C1=O] 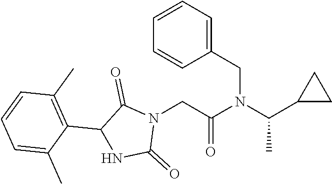 [SMILES: Cc1cccc(C)c1C1NC(=O)N(CC(=O)N(Cc2ccccc2)[C@@H](C)C2CC2)C1=O] 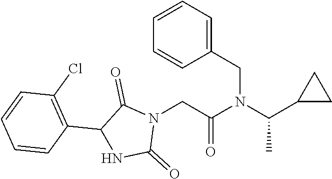 [SMILES: C[C@@H](C1CC1)N(Cc1ccccc1)C(=O)CN1C(=O)NC(c2ccccc2Cl)C1=O] 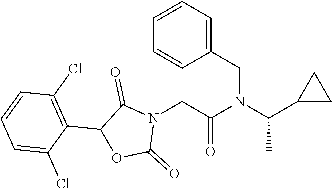 [SMILES: C[C@@H](C1CC1)N(Cc1ccccc1)C(=O)CN1C(=O)OC(c2c(Cl)cccc2Cl)C1=O] 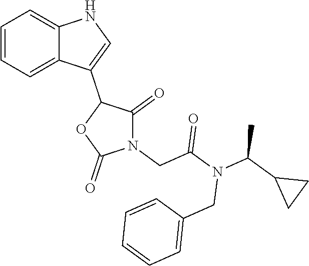 [SMILES: C[C@@H](C1CC1)N(Cc1ccccc1)C(=O)CN1C(=O)OC(c2c[nH]c3ccccc23)C1=O] 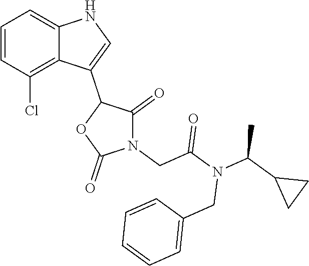 [SMILES: C[C@@H](C1CC1)N(Cc1ccccc1)C(=O)CN1C(=O)OC(c2c[nH]c3cccc(Cl)c23)C1=O] 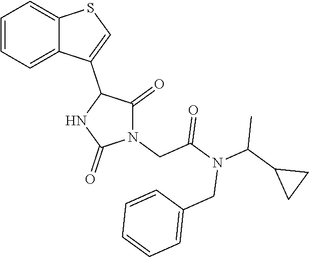 [SMILES: CC(C1CC1)N(Cc1ccccc1)C(=O)CN1C(=O)NC(c2csc3ccccc23)C1=O] 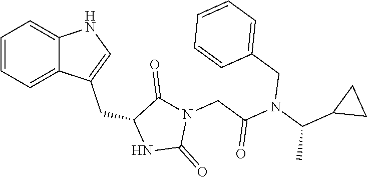 [SMILES: C[C@@H](C1CC1)N(Cc1ccccc1)C(=O)CN1C(=O)N[C@H](Cc2c[nH]c3ccccc23)C1=O] 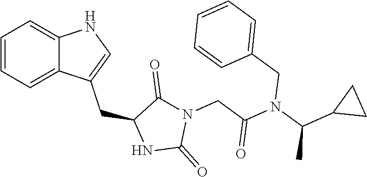 [SMILES: C[C@H](C1CC1)N(Cc1ccccc1)C(=O)CN1C(=O)N[C@@H](Cc2c[nH]c3ccccc23)C1=O]
TABLE 3-2 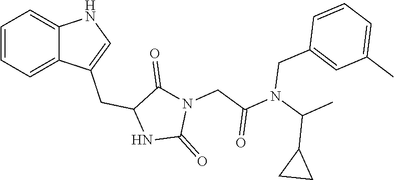 [SMILES: Cc1cccc(CN(C(=O)CN2C(=O)NC(Cc3c[nH]c4ccccc34)C2=O)C(C)C2CC2)c1]
or a pharmaceutically acceptable salt thereof.

38. The pharmaceutical composition of claim 31, wherein the low molecular weight compound is a compound represented by formula (4) 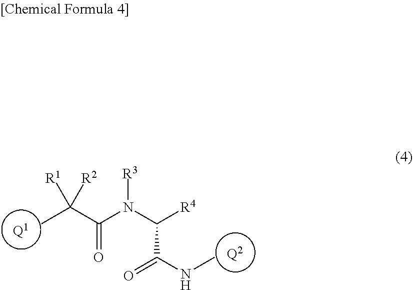
wherein
ring Q¹ represents a phenyl group optionally having 1 to 3 substituents independently selected from group A described below, or a 5- or 6-membered aromatic heterocyclic group having 1 to 3 nitrogen atoms having 1 to 3 substituents independently selected from group A described below within a ring,
ring Q² represents a phenyl group optionally having 1 to 3 substituents independently selected from group B described below, a naphthyl group optionally having 1 to 3 substituents independently selected from group B described below, a 5- or 6-membered aromatic heterocyclic group having 1 to 3 nitrogen atoms optionally having 1 to 3 substituents independently selected from group B described below within a ring, or a 8- to 10-membered bicyclic aromatic heterocyclic group optionally having 1 to 4 heteroatoms independently selected from the group consisting of a nitrogen atom, an oxygen atom, and a sulfur atom optionally having 1 to 3 substituents independently selected from group B described below within a ring,
R¹ and R² each independently represents a C₁₋₆ alkyl group or a C₁₋₆ alkoxy group, or
R¹ and R², together with the carbon atom to which R¹ and R² are attached, are a 3- to 7-membered cycloalkyl ring optionally having 1 to 3 substituents independently selected from group C described below, a tetrahydropyran ring optionally having 1 to 3 substituents independently selected from group C described below, or a dioxane ring optionally having 1 to 3 substituents independently selected from group C described below,
R³ represents a hydrogen atom, a C₁₋₆ alkyl group, or a hydroxy C₂₋₆ alkyl group,
R⁴ represents a hydrogen atom, a C₁₋₆ alkyl group, a hydroxy C₁₋₆ alkyl group, or a C₁₋₆ alkylsulfonyl C₁₋₆ alkyl group, or
R³ and R⁴, together with the nitrogen atom to which R³ is attached and the carbon atom to which R⁴ is attached, may form an azetidine ring optionally having 1 to 3 substituents independently selected from group D described below, a pyrrolidine ring optionally having 1 to 3 substituents independently selected from group D described below, a hexamethyleneimine ring optionally having 1 to 3 substituents independently selected from group D described below, a thiazolidine ring optionally having 1 to 3 substituents independently selected from group D described below, a 1-oxothiazolidine ring optionally having 1 to 3 substituents independently selected from group D described below, a 1,1′-dioxothiazolidine ring optionally having 1 to 3 substituents independently selected from group D described below, or a 4-oxopyrrolidine ring optionally having 1 to 3 substituents independently selected from group D described below,
wherein group A is a halogen atom, a hydroxy group, a carboxy group, a C₁₋₆ alkyl group, a hydroxy C₁₋₆ alkyl group, a C₁₋₆ alkoxy group, a halogeno C₁₋₆ alkoxy group, a C₁₋₆ alkoxycarbonyl group, a C₂₋₇ alkanoyl group, a halogeno C₂₋₇ alkanoyl group, a C₂₋₇ alkanoylamino group, a C₁₋₆ alkylsulfonyl group, a C₁₋₆ alkylsulfonylamino group, a C₃₋₇ cycloalkylsulfonylamino group, a phenyl group, a phenylsulfonylamino group, a carbamoyl group, a C₁₋₆ alkylcarbamoyl group, a di-C₁₋₆ alkylcarbamoyl group, a benzyloxycarbonyl group, a C₃₋₇ cycloalkylsulfonylcarbamoyl group, a halogeno C₁₋₆ alkylsulfonyloxy group, and a phenylsulfonyl group,
group B is a halogen atom, a cyano group, an amino group, a C₁₋₆ alkyl group, a C₁₋₆ alkoxy group, a hydroxy C₁₋₆ alkyl group, a C₁₋₆ alkylamino group, a C₁₋₆ alkylamino C₁₋₆ alkyl group, a morpholinyl C₁₋₆ alkyloxy group, a phenyl group, a benzyloxy group, a C₁₋₆ alkoxy C₁₋₆ alkyl group, a hydroxy group, a halogeno C₁₋₆ alkyl group, a C₁₋₆ alkoxycarbonyl group, a C₂₋₇ alkanoylamino group, a halogeno C₁₋₆ alkoxy group, a C₁₋₆ alkoxy C₁₋₆ alkoxy group, a C₁₋₆ alkylsulfonylamino group, a morpholinyl C₁₋₅ alkyl group, and a C₁₋₆ alkylsulfonyl group,
group C is a halogen atom, a C₁₋₆ alkyl group, and a C₁₋₆ alkoxy group,
group D is a halogen atom, a hydroxy group, a C₁₋₆ alkyl group, a C₁₋₆ alkoxy group, a C₁₋₆ alkoxy C₁₋₆ alkoxy group, a C₂₋₆ alkynyl group, a C₂₋₇ alkanoylamino group, an amino group, and a di-C₁₋₆ alkylamino group,
or a prodrug thereof or a pharmaceutically acceptable salt thereof.

39. The pharmaceutical composition of claim 31, wherein the low molecular weight compound is a compound represented by the following (Table 4)
TABLE 4 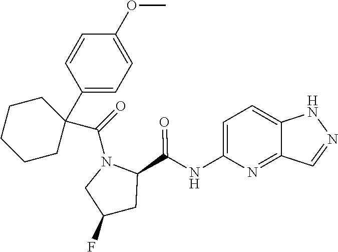 [SMILES: COc1ccc(C2(C(=O)N3C[C@H](F)C[C@@H]3C(=O)Nc3ccc4[nH]ncc4n3)CCCCC2)cc1] 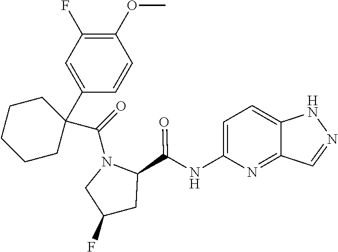 [SMILES: COc1ccc(C2(C(=O)N3C[C@H](F)C[C@@H]3C(=O)Nc3ccc4[nH]ncc4n3)CCCCC2)cc1F] 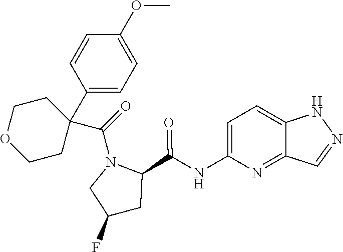 [SMILES: COc1ccc(C2(C(=O)N3C[C@H](F)C[C@@H]3C(=O)Nc3ccc4[nH]ncc4n3)CCOCC2)cc1] 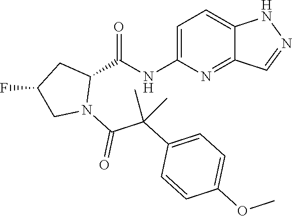 [SMILES: COc1ccc(C(C)(C)C(=O)N2C[C@H](F)C[C@@H]2C(=O)Nc2ccc3[nH]ncc3n2)cc1] 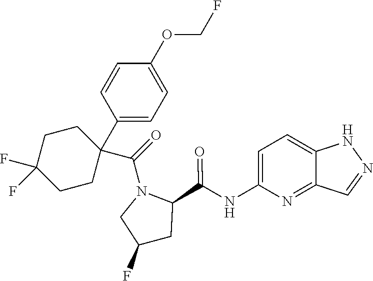 [SMILES: O=C(Nc1ccc2[nH]ncc2n1)[C@H]1C[C@@H](F)CN1C(=O)C1(c2ccc(OCF)cc2)CCC(F)(F)CC1] 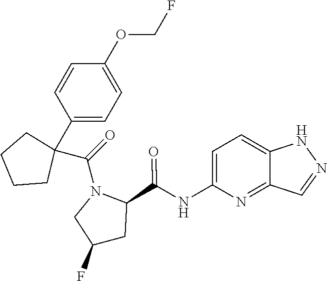 [SMILES: O=C(Nc1ccc2[nH]ncc2n1)[C@H]1C[C@@H](F)CN1C(=O)C1(c2ccc(OCF)cc2)CCCC1] 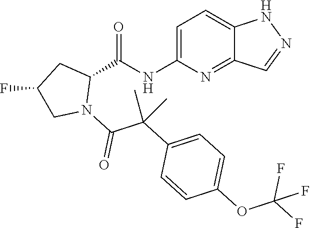 [SMILES: CC(C)(C(=O)N1C[C@H](F)C[C@@H]1C(=O)Nc1ccc2[nH]ncc2n1)c1ccc(OC(F)(F)F)cc1] 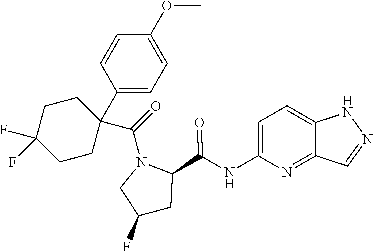 [SMILES: COc1ccc(C2(C(=O)N3C[C@H](F)C[C@@H]3C(=O)Nc3ccc4[nH]ncc4n3)CCC(F)(F)CC2)cc1] 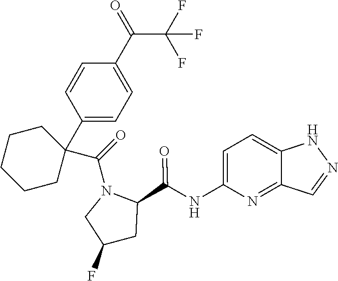 [SMILES: O=C(Nc1ccc2[nH]ncc2n1)[C@H]1C[C@@H](F)CN1C(=O)C1(c2ccc(C(=O)C(F)(F)F)cc2)CCCCC1] 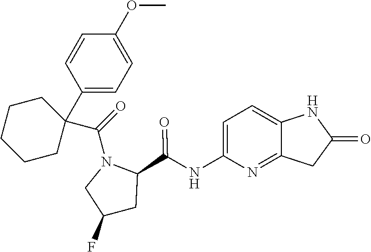 [SMILES: COc1ccc(C2(C(=O)N3C[C@H](F)C[C@@H]3C(=O)Nc3ccc4c(n3)CC(=O)N4)CCCCC2)cc1] 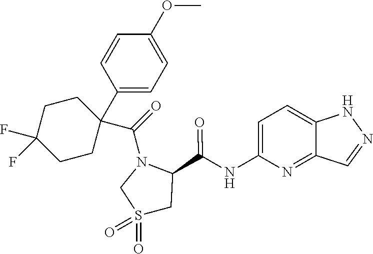 [SMILES: COc1ccc(C2(C(=O)N3CS(=O)(=O)C[C@@H]3C(=O)Nc3ccc4[nH]ncc4n3)CCC(F)(F)CC2)cc1] 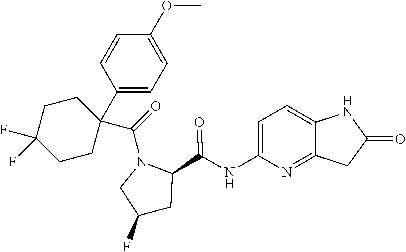 [SMILES: COc1ccc(C2(C(=O)N3C[C@H](F)C[C@@H]3C(=O)Nc3ccc4c(n3)CC(=O)N4)CCC(F)(F)CC2)cc1] 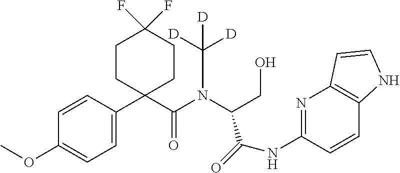 [SMILES: [2H]C([2H])([2H])N(C(=O)C1(c2ccc(OC)cc2)CCC(F)(F)CC1)[C@H](CO)C(=O)Nc1ccc2[nH]ccc2n1] 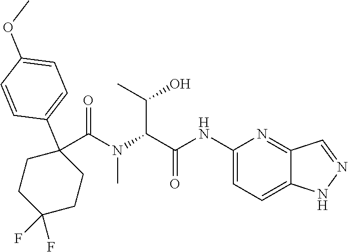 [SMILES: COc1ccc(C2(C(=O)N(C)[C@@H](C(=O)Nc3ccc4[nH]ncc4n3)[C@H](C)O)CCC(F)(F)CC2)cc1] 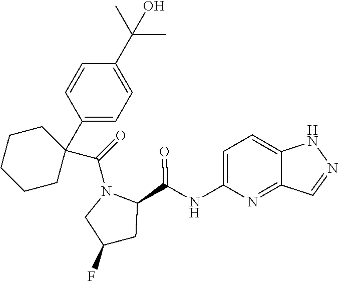 [SMILES: CC(C)(O)c1ccc(C2(C(=O)N3C[C@H](F)C[C@@H]3C(=O)Nc3ccc4[nH]ncc4n3)CCCCC2)cc1] 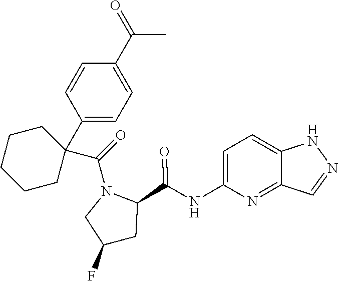 [SMILES: CC(=O)c1ccc(C2(C(=O)N3C[C@H](F)C[C@@H]3C(=O)Nc3ccc4[nH]ncc4n3)CCCCC2)cc1] 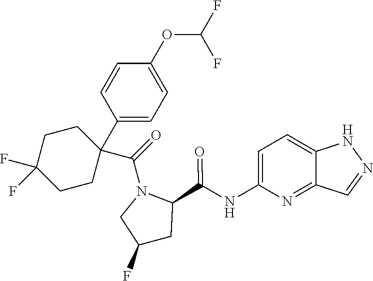 [SMILES: O=C(Nc1ccc2[nH]ncc2n1)[C@H]1C[C@@H](F)CN1C(=O)C1(c2ccc(OC(F)F)cc2)CCC(F)(F)CC1] 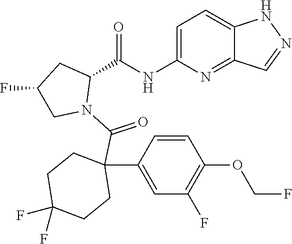 [SMILES: O=C(Nc1ccc2[nH]ncc2n1)[C@H]1C[C@@H](F)CN1C(=O)C1(c2ccc(OCF)c(F)c2)CCC(F)(F)CC1] 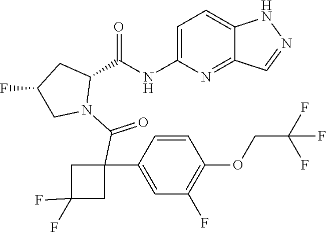 [SMILES: O=C(Nc1ccc2[nH]ncc2n1)[C@H]1C[C@@H](F)CN1C(=O)C1(c2ccc(OCC(F)(F)F)c(F)c2)CC(F)(F)C1] 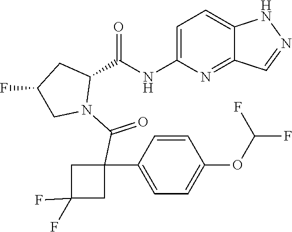 [SMILES: O=C(Nc1ccc2[nH]ncc2n1)[C@H]1C[C@@H](F)CN1C(=O)C1(c2ccc(OC(F)F)cc2)CC(F)(F)C1] 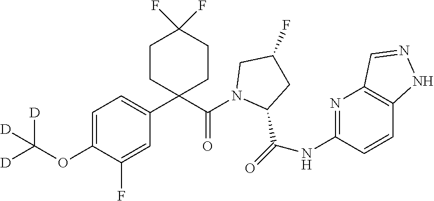 [SMILES: [2H]C([2H])([2H])Oc1ccc(C2(C(=O)N3C[C@H](F)C[C@@H]3C(=O)Nc3ccc4[nH]ncc4n3)CCC(F)(F)CC2)cc1F] 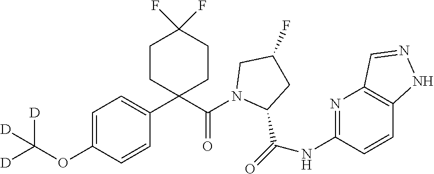 [SMILES: [2H]C([2H])([2H])Oc1ccc(C2(C(=O)N3C[C@H](F)C[C@@H]3C(=O)Nc3ccc4[nH]ncc4n3)CCC(F)(F)CC2)cc1] 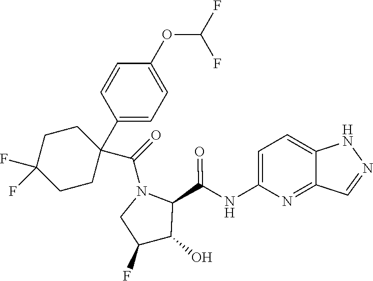 [SMILES: O=C(Nc1ccc2[nH]ncc2n1)[C@H]1[C@H](O)[C@@H](F)CN1C(=O)C1(c2ccc(OC(F)F)cc2)CCC(F)(F)CC1] 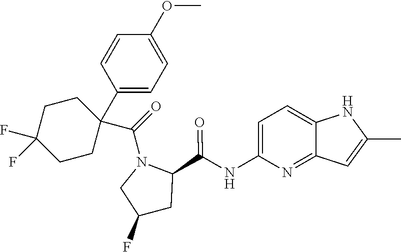 [SMILES: COc1ccc(C2(C(=O)N3C[C@H](F)C[C@@H]3C(=O)Nc3ccc4[nH]c(C)cc4n3)CCC(F)(F)CC2)cc1] 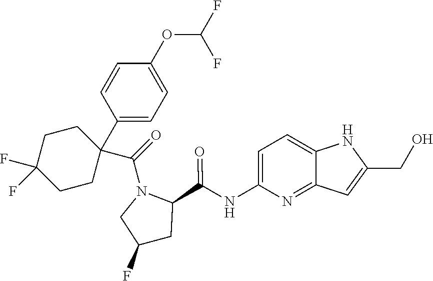 [SMILES: O=C(Nc1ccc2[nH]c(CO)cc2n1)[C@H]1C[C@@H](F)CN1C(=O)C1(c2ccc(OC(F)F)cc2)CCC(F)(F)CC1] 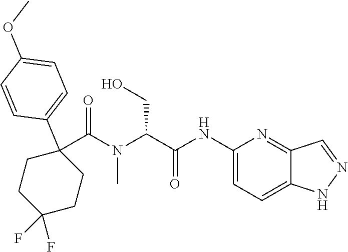 [SMILES: COc1ccc(C2(C(=O)N(C)[C@H](CO)C(=O)Nc3ccc4[nH]ncc4n3)CCC(F)(F)CC2)cc1] 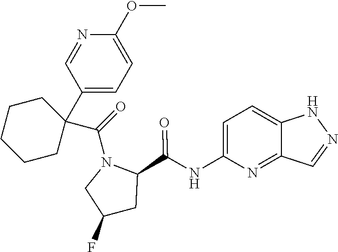 [SMILES: COc1ccc(C2(C(=O)N3C[C@H](F)C[C@@H]3C(=O)Nc3ccc4[nH]ncc4n3)CCCCC2)cn1]
or a pharmaceutically acceptable salt thereof.

40. The pharmaceutical composition of claim 31, wherein the low molecular weight compound is a compound represented by formula (5) 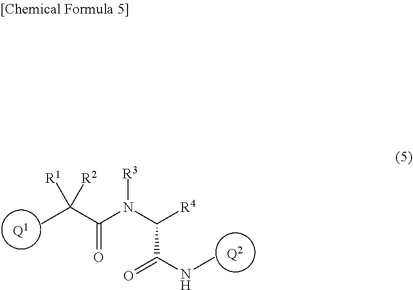
wherein
ring Q¹ represents a 3- to 7-membered cycloalkyl group optionally having 1 to 3 substituents independently selected from group A described below, a 3- to 7-membered heterocycloalkyl group having 1 to 2 heteroatoms independently selected from the group consisting of a nitrogen atom, an oxygen atom, and a sulfur atom optionally having 1 to 3 substituents independently selected from group A described below within a ring, or an 8- to 10-membered bicyclic heterocycloalkyl group having 1 to 3 heteroatoms independently selected from the group consisting of a nitrogen atom, an oxygen atom, and a sulfur atom optionally having 1 to 3 substituents independently selected from group A described below within a ring,
ring Q² represents a phenyl group optionally having 1 to 3 substituents independently selected from group B described below, a naphthyl group optionally having 1 to 3 substituents independently selected from group B described below, a 5- or 6-membered aromatic heterocyclic group having 1 to 3 nitrogen atoms optionally having 1 to 3 substituents independently selected from group B described below within a ring, or an 8- to 10-membered bicyclic aromatic heterocyclic group having 1 to 4 heteroatoms independently selected from the group consisting of a nitrogen atom, an oxygen atom, and a sulfur atom optionally having 1 to 3 substituents independently selected from group B described below within a ring,
R¹ and R² each independently represents a C₁₋₆ alkyl group or a C₁₋₆ alkoxy group, or
R¹ and R², together with the carbon atom to which R¹ and R² are attached, represent a 3- to 7-membered cycloalkyl ring optionally having 1 to 3 substituents independently selected from group C described below, a tetrahydropyran ring optionally having 1 to 3 substituents independently selected from group C described below, or a dioxane ring optionally having 1 to 3 substituents independently selected from group C described below,
R³ represents a hydrogen atom, a C₁₋₆ alkyl group, or a hydroxy C₂₋₆ alkyl group,
R⁴ represents a hydrogen atom, a C₁₋₆ alkyl group, a hydroxy C₁₋₆ alkyl group, or a C₁₋₆ alkylsulfonyl C₁₋₆ alkyl group, or
R³ and R⁴, together with the nitrogen atom to which R³ is attached and the carbon atom to which R⁴ is attached, may form an azetidine ring optionally having 1 to 3 substituents independently selected from group D described below, a pyrrolidine ring optionally having 1 to 3 substituents independently selected from group D described below, a hexamethyleneimine ring optionally having 1 to 3 substituents independently selected from group D described below, a thiazolidine ring optionally having 1 to 3 substituents independently selected from group D described below, a 1-oxothiazolidine ring optionally having 1 to 3 substituents independently selected from group D described below, a 1,1-dioxothiazolidine ring optionally having 1 to 3 substituents independently selected from group D described below, or a 4-oxopyrrolidine ring optionally having 1 to 3 substituents independently selected from group D described below,
wherein group A is a halogen atom, a hydroxy group, a carboxy group, an amino group, a C₁₋₆ alkyl group, a halogeno C₁₋₆ alkyl group, a hydroxy C₁₋₆ alkyl group, a C₁₋₆ alkoxy C₁₋₆ alkyl group, a C₁₋₆ alkoxy group, a halogeno C₁₋₆ alkoxy group, a C₁₋₆ alkoxy C₁₋₆ alkoxy group, a C₂₋₇ alkanoyl group, a hydroxy C₂₋₇ alkanoyl group, a C₂₋₇ alkanoylamino group, a C₁₋₆ alkylsulfonyl group, a C₁₋₆ alkylsulfonylamino group, a benzyl group, a benzyloxy group, and an oxo group,
group B is a halogen atom, a cyano group, an amino group, a C₁₋₆ alkyl group, a C₁₋₆ alkoxy group, a hydroxy C₁₋₆ alkyl group, a C₁₋₆ alkylamino group, a C₁₋₆ alkylamino C₁₋₆ alkyl group, a morpholinyl C₁₋₆ alkyloxy group, a phenyl group, a benzyloxy group, a C₁₋₆ alkoxy C₁₋₆ alkyl group, a hydroxy group, a halogeno C₁₋₆ alkyl group, a C₁₋₆ alkoxycarbonyl group, a C₂₋₇ alkanoylamino group, a halogeno C₁₋₆ alkoxy group, a C₁₋₆ alkoxy C₁₋₆ alkoxy group, a C₁₋₆ alkylsulfonylamino group, a morpholinyl C₁₋₆ alkyl group, and a C₁₋₆ alkylsulfonyl group,
group C is a halogen atom, a C₁₋₆ alkyl group, and a C₁₋₆ alkoxy group, and
group D is a halogen atom, a hydroxy group, a C₁₋₆ alkyl group, a C₁₋₆ alkoxy group, a C₁₋₆ alkoxy C₁₋₆ alkoxy group, a C₂₋₆ alkynyl group, a C₂₋₇ alkanoylamino group, an amino group, and a di-C₁₋₆ alkylamino group,
or a prodrug thereof or a pharmaceutically acceptable salt thereof.

41. The pharmaceutical composition of claim 31, wherein the low molecular weight compound is a compound represented by the following (Table 5)
TABLE 5 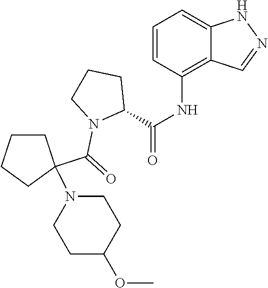 [SMILES: COC1CCN(C2(C(=O)N3CCC[C@@H]3C(=O)Nc3cccc4[nH]ncc34)CCCC2)CC1] 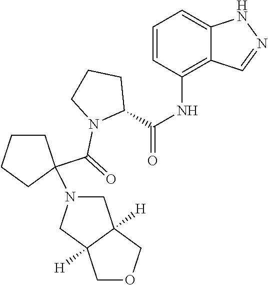 [SMILES: O=C(Nc1cccc2[nH]ncc12)[C@H]1CCCN1C(=O)C1(N2C[C@H]3COC[C@H]3C2)CCCC1] 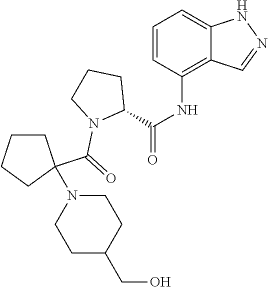 [SMILES: O=C(Nc1cccc2[nH]ncc12)[C@H]1CCCN1C(=O)C1(N2CCC(CO)CC2)CCCC1] 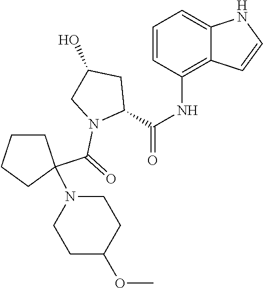 [SMILES: COC1CCN(C2(C(=O)N3C[C@H](O)C[C@@H]3C(=O)Nc3cccc4[nH]ccc34)CCCC2)CC1] 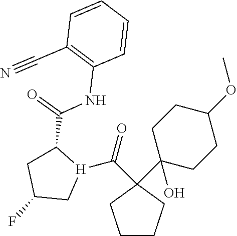 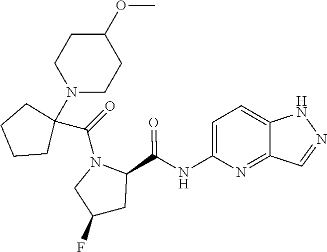 [SMILES: COC1CCN(C2(C(=O)N3C[C@H](F)C[C@@H]3C(=O)Nc3ccc4[nH]ncc4n3)CCCC2)CC1] 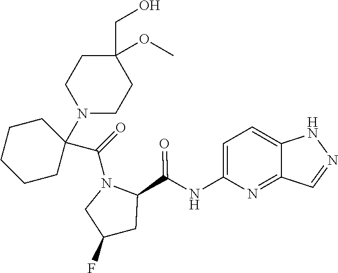 [SMILES: COC1(CO)CCN(C2(C(=O)N3C[C@H](F)C[C@@H]3C(=O)Nc3ccc4[nH]ncc4n3)CCCCC2)CC1] 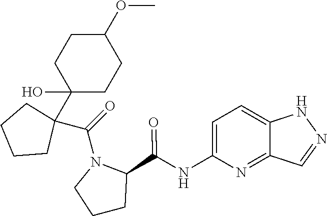 [SMILES: COC1CCC(O)(C2(C(=O)N3CCC[C@@H]3C(=O)Nc3ccc4[nH]ncc4n3)CCCC2)CC1] 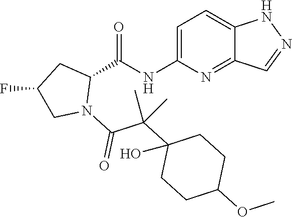 [SMILES: COC1CCC(O)(C(C)(C)C(=O)N2C[C@H](F)C[C@@H]2C(=O)Nc2ccc3[nH]ncc3n2)CC1] 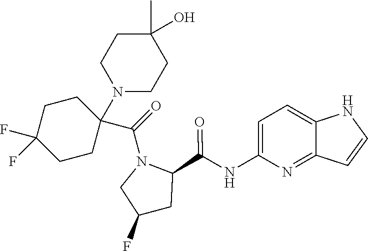 [SMILES: CC1(O)CCN(C2(C(=O)N3C[C@H](F)C[C@@H]3C(=O)Nc3ccc4[nH]ccc4n3)CCC(F)(F)CC2)CC1] 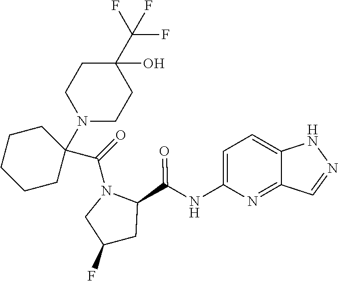 [SMILES: O=C(Nc1ccc2[nH]ncc2n1)[C@H]1C[C@@H](F)CN1C(=O)C1(N2CCC(O)(C(F)(F)F)CC2)CCCCC1] 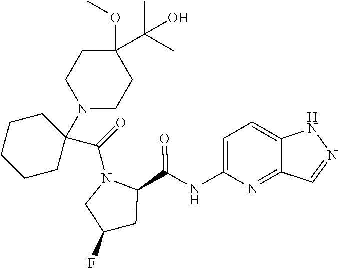 [SMILES: COC1(C(C)(C)O)CCN(C2(C(=O)N3C[C@H](F)C[C@@H]3C(=O)Nc3ccc4[nH]ncc4n3)CCCCC2)CC1] 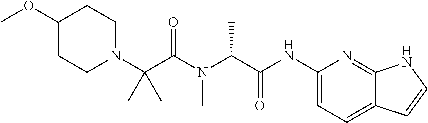 [SMILES: COC1CCN(C(C)(C)C(=O)N(C)[C@H](C)C(=O)Nc2ccc3cc[nH]c3n2)CC1] 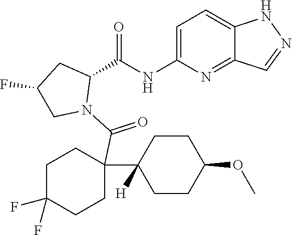 [SMILES: CO[C@H]1CC[C@H](C2(C(=O)N3C[C@H](F)C[C@@H]3C(=O)Nc3ccc4[nH]ncc4n3)CCC(F)(F)CC2)CC1] 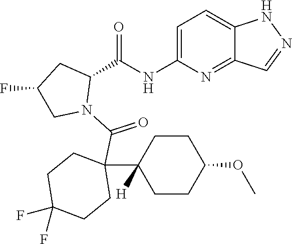 [SMILES: CO[C@H]1CC[C@@H](C2(C(=O)N3C[C@H](F)C[C@@H]3C(=O)Nc3ccc4[nH]ncc4n3)CCC(F)(F)CC2)CC1] 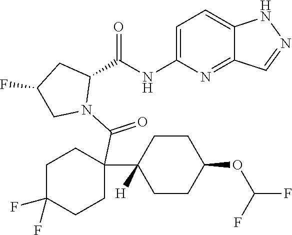 [SMILES: O=C(Nc1ccc2[nH]ncc2n1)[C@H]1C[C@@H](F)CN1C(=O)C1([C@H]2CC[C@H](OC(F)F)CC2)CCC(F)(F)CC1] 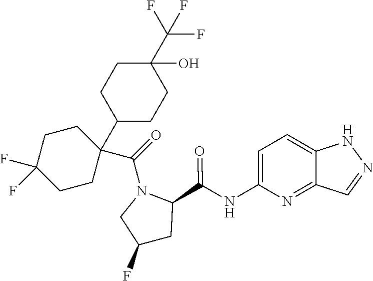 [SMILES: O=C(Nc1ccc2[nH]ncc2n1)[C@H]1C[C@@H](F)CN1C(=O)C1(C2CCC(O)(C(F)(F)F)CC2)CCC(F)(F)CC1] 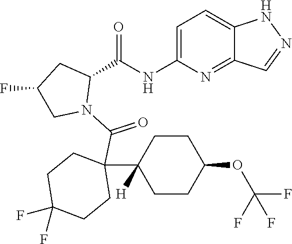 [SMILES: O=C(Nc1ccc2[nH]ncc2n1)[C@H]1C[C@@H](F)CN1C(=O)C1([C@H]2CC[C@H](OC(F)(F)F)CC2)CCC(F)(F)CC1]
or a pharmaceutically acceptable salt thereof.

42. The pharmaceutical composition of claim 31, wherein the low molecular weight compound is a compound represented by formula (6) 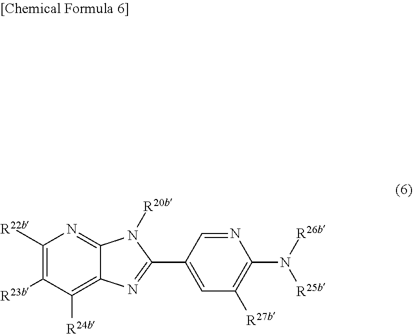
wherein
R^{20b′} is C₁₋₂ alkyl (wherein the alkyl group is substituted with phenyl substituted with pyrimidinyl, pyrazolyl, pyrazolyl substituted with C₁₋₃ alkyl, pyrazinyl, pyrazinyl substituted with C₁₋₃ alkyl, piperazinyl, piperazinyl substituted with oxo, piperazinyl substituted with C₁₋₃ alkyl, oxazolyl, oxazolyl substituted with C₁₋₃ alkyl, imidazolyl, imidazolyl substituted with C₁₋₃ alkyl, morpholinyl, morpholinyl substituted with 1 to 2 C₁₋₃ alkyl, morpholinyl substituted with oxo, dioxanyl, dioxanyl substituted with C₁₋₃ alkyl, 4,5,6,7-tetrahydropyrazolo[1,5-a]pyrazine, triazolyl, triazolyl substituted with C₁₋₃ alkyl, thiazolyl, thiazolyl substituted with C₁₋₃ alkyl, cyclopentyloxy, C₁₋₆ alkoxy, C₁₋₆ alkoxy substituted with 1 to 6 fluoro, C₁₋₆ alkoxy substituted with hydroxy, tetrahydrofuran, pyridyl, pyridyl substituted with bromo, or pyridyl substituted with pyrimidinyl);
R^{22b′}, R^{23b′}, and R^{24b′} are each independently selected from a hydrogen atom, fluoro, chloro, bromo, —OH, boronic acid, 1,3,6,2-dioxazaborocane-4,8-dione, —CN, —C(O)NHCH₃, —C(O)NHCH₂CH₃, —C(O)NHCH₂CF₂H, —C(O)NHCH₂CH₂OH, —C(O)NHCH₂CH₂SO₂CH₃, —C(O)NHOCH₃, —C(O)NH₂, —C(O)OCH₃, —C(O)NHCH₂ cyclopropyl, —C(O)NH cyclobutyl (wherein the group is optionally substituted with hydroxy), —CH₂ morpholinyl, —CH₂OH, —CH₂NHCH₂CF₃, —CH₂NHCH₂CH₂SO₂CH₃, —CH₂SO₂CH₃, —CH(OH)CF₃, —CH₃, —CF₃, —OCH₃, —OCD₃, —NHC(O)CH₃, —NH₂, —NHSO₂CH₃, morpholinyl, pyrazolyl, oxazolyl, and oxazolyl substituted with 1 to 2 methyl;
R^{23b′} and R^{24b′}, together with the carbon atom to which they are attached, may form oxaborolyl (wherein the group is optionally substituted with hydroxy);
R^{25b′} and R^{26b′} are each independently selected from C₁₋₃ alkyl, C₁₋₃ alkyl substituted with 1 to 3 fluoro, or cyclopropyl;
wherein R^{25b′} and R^{26b′}, together with the nitrogen atom to which they are attached, may form azetidinyl or pyrrolidinyl (wherein the group is optionally substituted with 1 to 2 C₁₋₃ alkyl, or C₁₋₃ alkyl substituted with 1 to 3 fluoro), or
one of R^{25b′} and R^{26b′} may form pyrrolidinyl or morpholinyl with R^{27b′} and any one heteroatom (wherein the group is optionally substituted with 1 to 4 C₁₋₃ alkyl);
R^{27b′} is selected from a hydrogen atom and fluoro;
wherein one of R^{25b′} and R^{26b′} may form pyrrolidinyl or morpholinyl with R^{27b′} and any one heteroatom (wherein the group is optionally substituted with 1 to 4 C₁₋₃ alkyl),
or a prodrug thereof or a pharmaceutically acceptable salt thereof.

43. The pharmaceutical composition of claim 31, wherein the low molecular weight compound is a compound represented by the following (Table 6)
TABLE 6 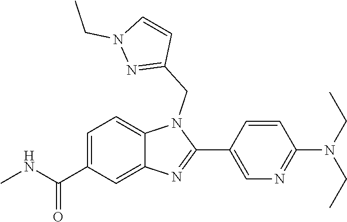 [SMILES: CCN(CC)c1ccc(-c2nc3cc(C(=O)NC)ccc3n2Cc2ccn(CC)n2)cn1] 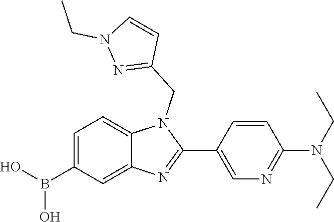 [SMILES: CCN(CC)c1ccc(-c2nc3cc(B(O)O)ccc3n2Cc2ccn(CC)n2)cn1] 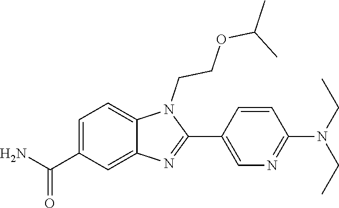 [SMILES: CCN(CC)c1ccc(-c2nc3cc(C(N)=O)ccc3n2CCOC(C)C)cn1] 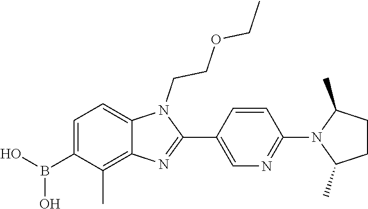 [SMILES: CCOCCn1c(-c2ccc(N3[C@@H](C)CC[C@@H]3C)nc2)nc2c(C)c(B(O)O)ccc21]
or a pharmaceutically acceptable salt thereof.

44. The pharmaceutical composition of claim 31, wherein the low molecular weight compound is a compound represented by formula (7) 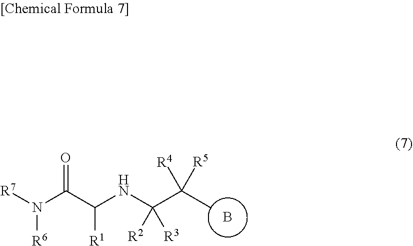
wherein
ring B is aryl, heterocyclyl, or heteroaryl (wherein the ring is each optionally substituted with 1 to 4 substituents selected from R^{b});
R⁶ is a hydrogen atom or C₁₋₆ alkyl;
R⁷ is aryl or heteroaryl (wherein the group is each substituted with a substituent selected from R^{f}, and optionally substituted with 1 to 4 substituents selected from R³);
wherein R⁶ and R⁷, together with the nitrogen ring to which they are attached, may form fused bicyclic heterocyclyl optionally substituted with 1 to 4 groups selected from R^{a};
R¹ is C₁₋₆ alkyl, C₁₋₆ haloalkyl, C₂₋₆ alkenyl, —C₁₋₆ alkyl OR^{c}, —C₁₋₆ alkyl N(R^{d})₂, —C₁₋₆ alkyl C(O)OR^{d}, —C₁₋₆ alkyl OC₁₋₆ alkyl N(R^{d})₂, —C₁₋₆ alkyl SOR^{d}, —C₁₋₆ alkyl S(O)₂R^{d}, —C₁₋₆ alkyl SON(R^{d})₂, —C₁₋₆ alkyl SO₂N(R^{d})₂, —C₁₋₆ alkylcycloalkyl, —C₁₋₆ alkylheterocyclyl, —C₁₋₆ alkylheteroaryl, —C₁₋₆ alkylaryl, cycloalkyl, aryl, heteroaryl, or heterocyclyl (wherein each of the cycloalkyl, heterocyclyl, aryl, and heteroaryl, alone or attached to —C₁₋₆ alkylcycloalkyl, —C₁₋₆ alkylheterocyclyl, —C₁₋₆ alkylheteroaryl, or —C₁₋₆ alkylaryl, is optionally substituted with 1 to 3 groups selected from R^{c});
R², R³, R⁴, and R⁵ are each independently a hydrogen atom or C₁₋₆ alkyl (wherein the C₁₋₆ alkyl is optionally substituted with 1 to 2 substituents selected from a halogen atom, —C(O)OR^{d}, —OC₁₋₆ alkyl N(R^{d})₂, —C₁₋₆ alkyl N(R^{d})₂, —N(R^{d})₂, —NR^{d}C₁₋₆ alkyl OR^{d}, —SOR^{d}, —S(O)₂R^{d}, —SON(R^{d})₂, —SO₂N(R^{d})₂, C₃₋₁₀ cycloalkyl, C₅₋₁₀ heterocyclyl, C₅₋₁₀ heteroaryl, and C₆₋₁₀ aryl);
R^{a}, R^{b}, and R^{c} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O)OR^{d}, —C(O)N(R^{d})₂, —C(O)NR^{d}C₁₋₆ alkyl OR^{d}, —OC₁₋₆ alkyl N(R^{d})₂, —C₁₋₆ alkyl C(O)N(R^{d})₂, —C₁₋₆ alkyl N(R^{d})₂, —N(R^{d})₂, —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)₂R^{d}, —SON(R^{d})₂, —SO₂N(R^{d})₂, —SF₅, —O— cycloalkyl, —O—C₁₋₄ alkyl-aryl, —C₁₋₆ alkylcycloalkyl, —C₁₋₆ alkylaryl, —C₁₋₆ alkylheteroaryl, —C₁₋₆ alkylheterocyclyl, cycloalkyl, heterocyclyl, heteroaryl, or aryl (wherein each of the cycloalkyl, heterocyclyl, aryl, and heteroaryl, alone or attached to —O-cycloalkyl, —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 groups selected from halogen, C₁₋₆ alkyl, C₁₋₆ haloalkyl, C₁₋₆ alkoxy, C₁₋₆ haloalkoxy, —N(R^{d}), —C(O)R^{d}, and —C₁₋₆ alkyl OR^{d});
R^{d} is independently a hydrogen atom, C₁₋₆ haloalkyl, or C₁₋₆ alkyl;
R^{f} is independently cycloalkyl, heterocyclyl, heteroaryl, or aryl (wherein each of the cycloalkyl, heterocyclyl, aryl, and heteroaryl is optionally substituted with 1 to 3 substituents selected from a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O)OR^{d}, —C(O)N(R^{d})₂, —C(O)NR^{d}C₁₋₆ alkyl OR^{d}, —OC₁₋₆ alkyl N(R^{d})₂, —C₁₋₆ alkyl C(O)N(R^{d})₂, —C₁₋₆ alkyl N(R^{d})₂, —N(R^{d})₂, —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)₂R^{d}, —SON(R^{d})₂, —SO₂N(R^{d})₂, —SF₅, and —O-cycloalkyl);
wherein the compound is not N-[1,1′-biphenyl]-2-yl-2-[[2-(3,4-dimethoxyphenyl)ethyl]amino]-propanamide, 2-[(2-phenylpropyl)amino]-N-[4-(1H-1,2,4-triazol-1-yl)phenyl]-propanamide, or a salt thereof,
or a prodrug thereof or a pharmaceutically acceptable salt thereof.

45. The pharmaceutical composition of claim 31, wherein the low molecular weight compound is a compound represented by the following (Table 7))
TABLE 7 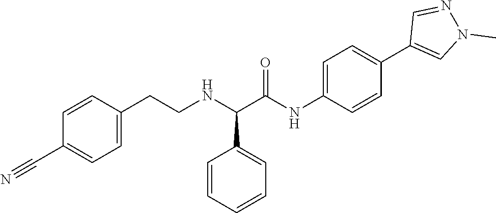 [SMILES: Cn1cc(-c2ccc(NC(=O)[C@H](NCCc3ccc(C#N)cc3)c3ccccc3)cc2)cn1] 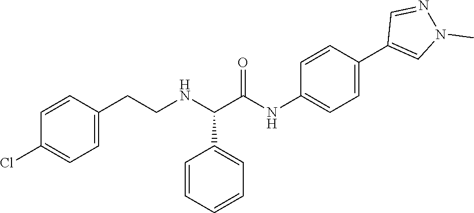 [SMILES: Cn1cc(-c2ccc(NC(=O)[C@@H](NCCc3ccc(Cl)cc3)c3ccccc3)cc2)cn1] 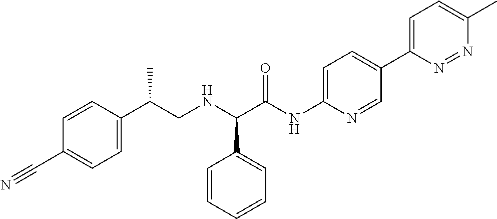 [SMILES: Cc1ccc(-c2ccc(NC(=O)[C@H](NC[C@@H](C)c3ccc(C#N)cc3)c3ccccc3)nc2)nn1] 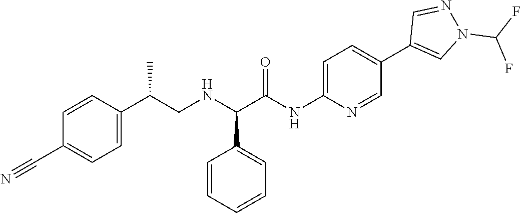 [SMILES: C[C@H](CN[C@@H](C(=O)Nc1ccc(-c2cnn(C(F)F)c2)cn1)c1ccccc1)c1ccc(C#N)cc1] 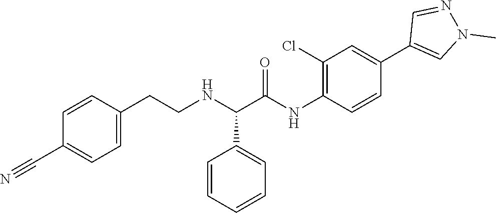 [SMILES: Cn1cc(-c2ccc(NC(=O)[C@@H](NCCc3ccc(C#N)cc3)c3ccccc3)c(Cl)c2)cn1] 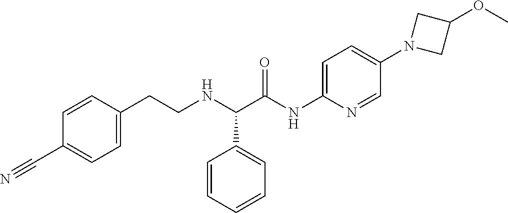 [SMILES: COC1CN(c2ccc(NC(=O)[C@@H](NCCc3ccc(C#N)cc3)c3ccccc3)nc2)C1] 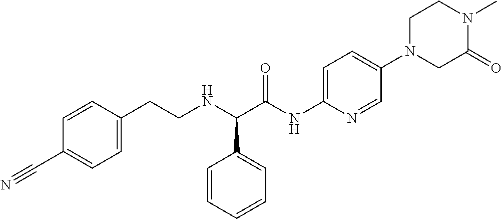 [SMILES: CN1CCN(c2ccc(NC(=O)[C@H](NCCc3ccc(C#N)cc3)c3ccccc3)nc2)CC1=O] 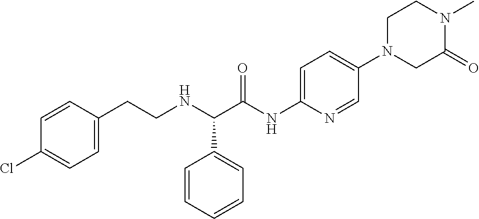 [SMILES: CN1CCN(c2ccc(NC(=O)[C@@H](NCCc3ccc(Cl)cc3)c3ccccc3)nc2)CC1=O] 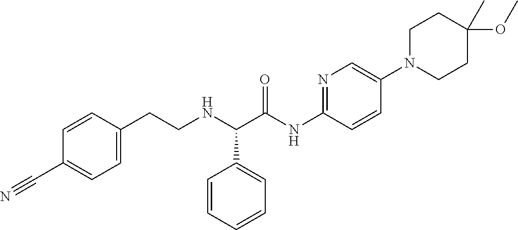 [SMILES: COC1(C)CCN(c2ccc(NC(=O)[C@@H](NCCc3ccc(C#N)cc3)c3ccccc3)nc2)CC1] 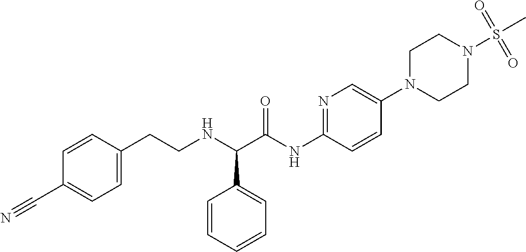 [SMILES: CS(=O)(=O)N1CCN(c2ccc(NC(=O)[C@H](NCCc3ccc(C#N)cc3)c3ccccc3)nc2)CC1] 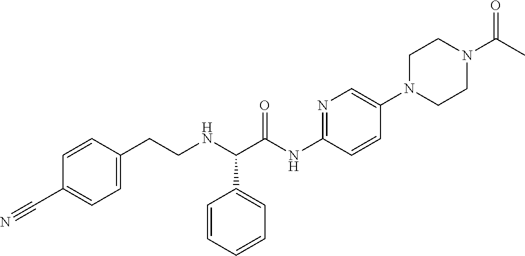 [SMILES: CC(=O)N1CCN(c2ccc(NC(=O)[C@@H](NCCc3ccc(C#N)cc3)c3ccccc3)nc2)CC1] 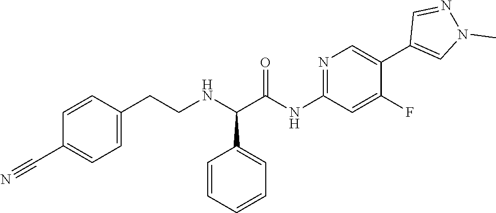 [SMILES: Cn1cc(-c2cnc(NC(=O)[C@H](NCCc3ccc(C#N)cc3)c3ccccc3)cc2F)cn1] 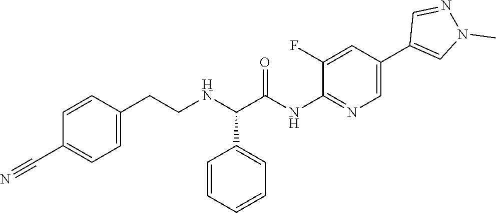 [SMILES: Cn1cc(-c2cnc(NC(=O)[C@@H](NCCc3ccc(C#N)cc3)c3ccccc3)c(F)c2)cn1] 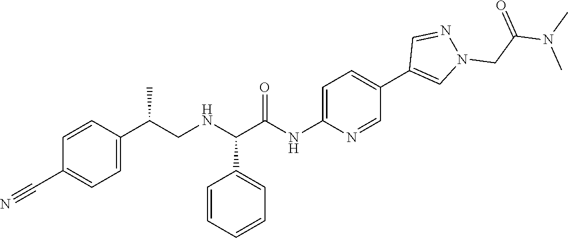 [SMILES: C[C@H](CN[C@H](C(=O)Nc1ccc(-c2cnn(CC(=O)N(C)C)c2)cn1)c1ccccc1)c1ccc(C#N)cc1] 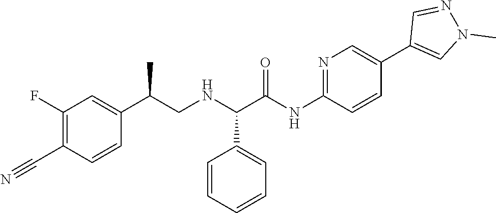 [SMILES: C[C@@H](CN[C@H](C(=O)Nc1ccc(-c2cnn(C)c2)cn1)c1ccccc1)c1ccc(C#N)c(F)c1] 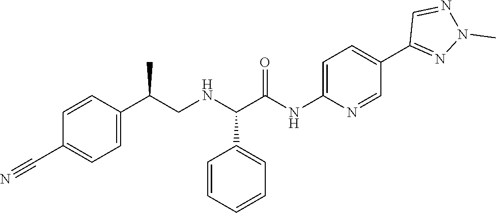 [SMILES: C[C@@H](CN[C@H](C(=O)Nc1ccc(-c2cnn(C)n2)cn1)c1ccccc1)c1ccc(C#N)cc1] 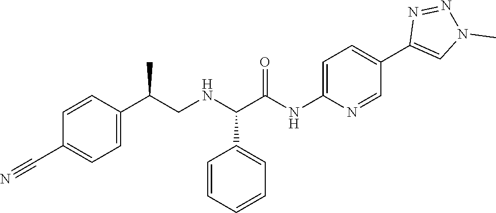 [SMILES: C[C@@H](CN[C@H](C(=O)Nc1ccc(-c2cn(C)nn2)cn1)c1ccccc1)c1ccc(C#N)cc1] 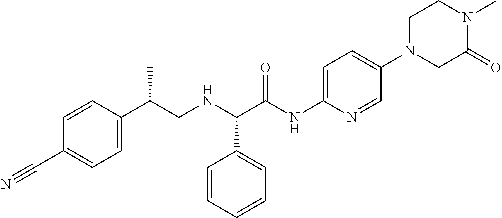 [SMILES: C[C@H](CN[C@H](C(=O)Nc1ccc(N2CCN(C)C(=O)C2)cn1)c1ccccc1)c1ccc(C#N)cc1] 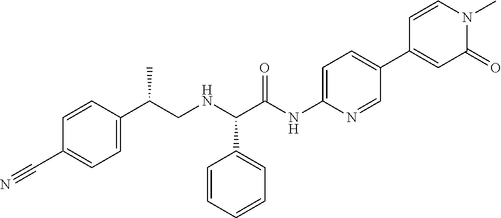 [SMILES: C[C@H](CN[C@H](C(=O)Nc1ccc(-c2ccn(C)c(=O)c2)cn1)c1ccccc1)c1ccc(C#N)cc1] 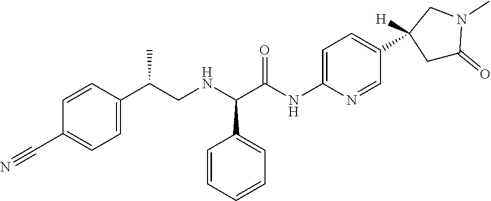 [SMILES: C[C@H](CN[C@@H](C(=O)Nc1ccc([C@H]2CC(=O)N(C)C2)cn1)c1ccccc1)c1ccc(C#N)cc1] 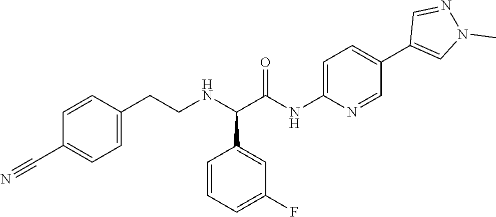 [SMILES: Cn1cc(-c2ccc(NC(=O)[C@H](NCCc3ccc(C#N)cc3)c3cccc(F)c3)nc2)cn1] 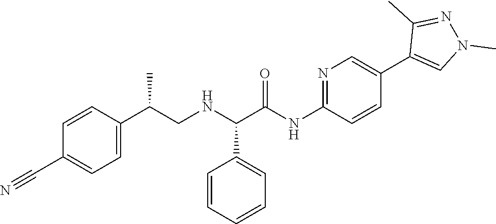 [SMILES: Cc1nn(C)cc1-c1ccc(NC(=O)[C@@H](NC[C@@H](C)c2ccc(C#N)cc2)c2ccccc2)nc1] 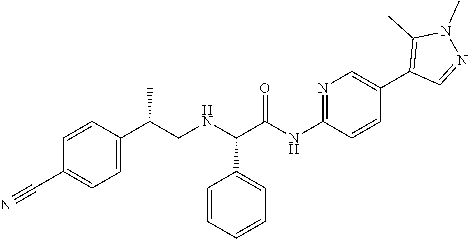 [SMILES: Cc1c(-c2ccc(NC(=O)[C@@H](NC[C@@H](C)c3ccc(C#N)cc3)c3ccccc3)nc2)cnn1C] 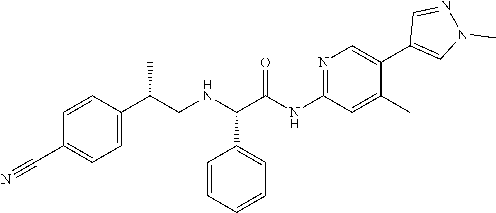 [SMILES: Cc1cc(NC(=O)[C@@H](NC[C@@H](C)c2ccc(C#N)cc2)c2ccccc2)ncc1-c1cnn(C)c1] 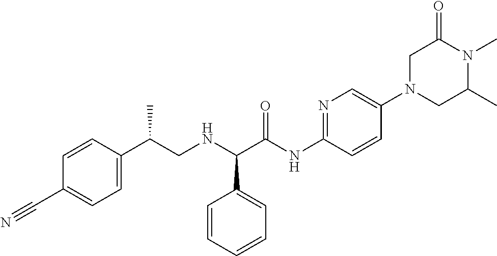 [SMILES: CC1CN(c2ccc(NC(=O)[C@H](NC[C@@H](C)c3ccc(C#N)cc3)c3ccccc3)nc2)CC(=O)N1C] 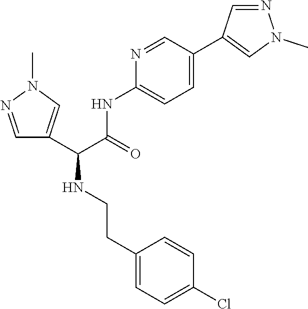 [SMILES: Cn1cc(-c2ccc(NC(=O)[C@@H](NCCc3ccc(Cl)cc3)c3cnn(C)c3)nc2)cn1]
or a pharmaceutically acceptable salt thereof.

46. The pharmaceutical composition of claim 31, wherein the low molecular weight compound is a compound represented by formula (8) 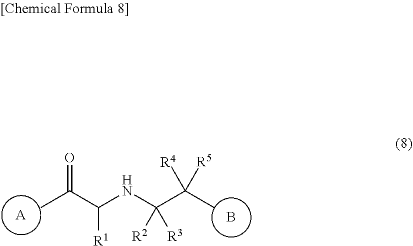
wherein
ring A is bicyclic heteroaryl optionally substituted with 1 to 4 substituents selected from R^{a};
ring B is aryl, heterocyclyl, or heteroaryl optionally substituted with 1 to 4 substituents selected from R^{b};
R¹ is C₁₋₆ alkyl, C₁₋₆ haloalkyl, C₂₋₆ alkenyl, —C₁₋₆ alkyl OR^{c}, —C₁₋₆ alkyl N(R^{d})₂, —C₁₋₆ alkyl C(O)OR^{d}, —C₁₋₆ alkyl OC₁₋₆ alkyl N(R^{d})₂, —C₁₋₆ alkyl SOR^{d}, —C₁₋₆ alkyl S(O)₂R^{d}, —C₁₋₆ alkyl SON(R^{d})₂, —C₁₋₆ alkyl SO₂N(R^{d})₂, —C₁₋₆ alkylcycloalkyl, —C₁₋₆ alkylheterocyclyl, —C₁₋₆ alkylheteroaryl, —C₁₋₆ alkylaryl, cycloalkyl, aryl, heteroaryl, or heterocyclyl (wherein each of the cycloalkyl, heterocyclyl, aryl, and heteroaryl, alone or for —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 groups selected from R^{c});
R², R³, R⁴, and R⁵ are each independently a hydrogen atom or C₁₋₆ alkyl (wherein the C₁₋₆ alkyl is optionally substituted with 1 to 2 substituents selected from a halogen atom, —C(O)OR^{d}, —OC₁₋₆ alkyl N(R^{d})₂, —C₁₋₆ alkyl N(R^{d})₂, —N(R^{d})—, —NR^{d}C₁₋₆ alkyl OR^{d}, —SOR^{d}, —S(O)₂R^{d}, —SON(R^{d})₂, —SO₂N(R^{d})₂, cycloalkyl, heterocyclyl, heteroaryl, and aryl);
R^{a}, R^{b}, and R^{c} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{d}, —C(O)R^{d}, —C(O)OR^{d}, —C₁₋₆ alkyl C(O) OR^{d}, —C(O)N(R^{d})₂, —C(O)NR^{d}C₁₋₆ alkyl OR^{d}, —OC₁₋₆ alkyl N(R^{d}), C₁₋₆ alkyl C(O)N(R^{d})₂, —C₁₋₆ alkyl N(R^{d})₂, —N(R^{d}), —C(O)NR^{d}C₁₋₆ alkyl N(R^{d})₂, —NR^{d}C₁₋₆ alkyl N(R^{d})₂, —NR^{d}C₁₋₆ alkyl OR^{d}, —SOR^{d}, —S(O)—R^{d}, —SON(R^{d})₂, —SO₂N(R^{d})₂, —SF₅, —O— cycloalkyl, —O-heterocyclyl, —O—C₁₋₄ alkyl-aryl, —C₁₋₆ alkylcycloalkyl, —C₁₋₆ alkylaryl, —C₁₋₆ alkylheteroaryl, —C₁₋₆ alkylheterocyclyl, cycloalkyl, heterocyclyl, heteroaryl, or aryl (wherein each of the cycloalkyl, heterocyclyl, aryl, and heteroaryl, alone or attached to —O-cycloalkyl, —C₁₋₆ alkylcycloalkyl, —C₁₋₆ alkylaryl, —C₁₋₆ alkylheteroaryl, or —C₁₋₆ alkylheterocyclyl, is optionally substituted with 1 to 3 of a halogen atom, oxo, C₁₋₆ alkyl, C₁₋₆ haloalkyl, C₁₋₆ alkoxy, C₁₋₆ haloalkoxy, —N(R^{d})₂, —C(O)R^{d}, and —C₁₋₆ alkyl OR^{d});
R^{d} is each independently a hydrogen atom, heterocyclyl, C₁₋₆ haloalkyl, or C₁₋₆ alkyl, wherein the heterocyclyl is optionally substituted with 1 to 2 substituents selected from C₁₋₄ haloalkyl and C₁₋₄ alkyl, and the C₁₋₆ alkyl is optionally substituted with SO₂C₁₋₄ alkyl or heterocyclyl (wherein the group is optionally substituted with oxo);
wherein the compound is not 4-(2-((2-(1H-indol-3-yl)-2-oxo-1-phenylethyl)amino)ethyl)benzenesulfonamide, 4-[2-[[2-(7-ethyl-1H-indol-3-yl)-2-oxo-1-phenylethyl]amino]ethyl]benzenesulfonamide, 2-[[2-(3,4-dimethoxyphenyl)ethyl]amino]-1-(1H-indol-3-yl)-2-phenylethanone, or a salt thereof,
or a prodrug thereof or a pharmaceutically acceptable salt thereof.

47. The pharmaceutical composition of claim 31, wherein the low molecular weight compound is a compound represented by the following (Table 8)
TABLE 8 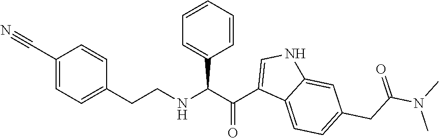 [SMILES: CN(C)C(=O)Cc1ccc2c(C(=O)[C@@H](NCCc3ccc(C#N)cc3)c3ccccc3)c[nH]c2c1] 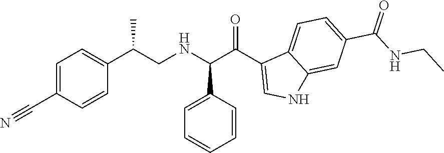 [SMILES: CCNC(=O)c1ccc2c(C(=O)[C@H](NC[C@@H](C)c3ccc(C#N)cc3)c3ccccc3)c[nH]c2c1] 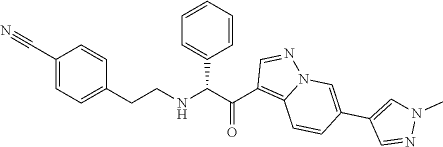 [SMILES: Cn1cc(-c2ccc3c(C(=O)[C@H](NCCc4ccc(C#N)cc4)c4ccccc4)cnn3c2)cn1] 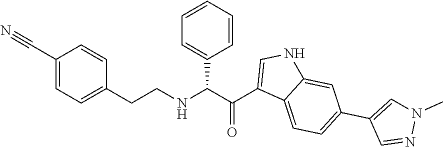 [SMILES: Cn1cc(-c2ccc3c(C(=O)[C@H](NCCc4ccc(C#N)cc4)c4ccccc4)c[nH]c3c2)cn1] 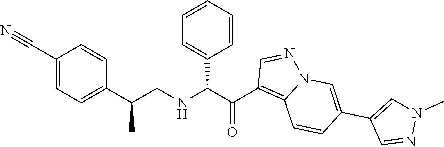 [SMILES: C[C@H](CN[C@@H](C(=O)c1cnn2cc(-c3cnn(C)c3)ccc12)c1ccccc1)c1ccc(C#N)cc1] 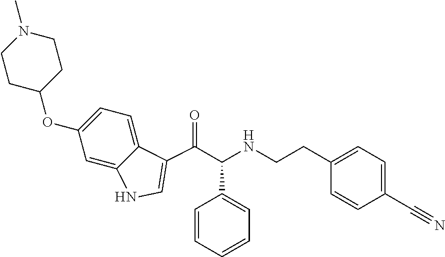 [SMILES: CN1CCC(Oc2ccc3c(C(=O)[C@H](NCCc4ccc(C#N)cc4)c4ccccc4)c[nH]c3c2)CC1] 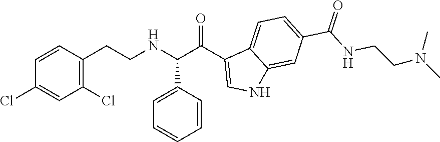 [SMILES: CN(C)CCNC(=O)c1ccc2c(C(=O)[C@@H](NCCc3ccc(Cl)cc3Cl)c3ccccc3)c[nH]c2c1] 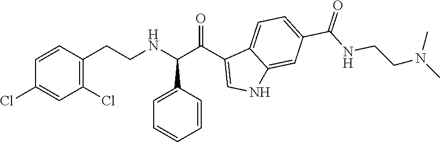 [SMILES: CN(C)CCNC(=O)c1ccc2c(C(=O)[C@H](NCCc3ccc(Cl)cc3Cl)c3ccccc3)c[nH]c2c1] 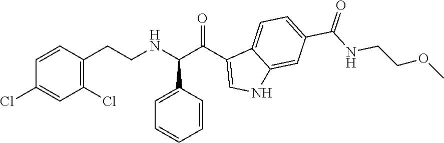 [SMILES: COCCNC(=O)c1ccc2c(C(=O)[C@H](NCCc3ccc(Cl)cc3Cl)c3ccccc3)c[nH]c2c1] 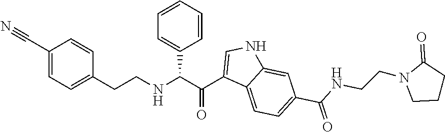 [SMILES: N#Cc1ccc(CCN[C@@H](C(=O)c2c[nH]c3cc(C(=O)NCCN4CCCC4=O)ccc23)c2ccccc2)cc1]
or a pharmaceutically acceptable salt thereof.

48. The pharmaceutical composition of claim 31, wherein the low molecular weight compound is a compound represented by formula (9) 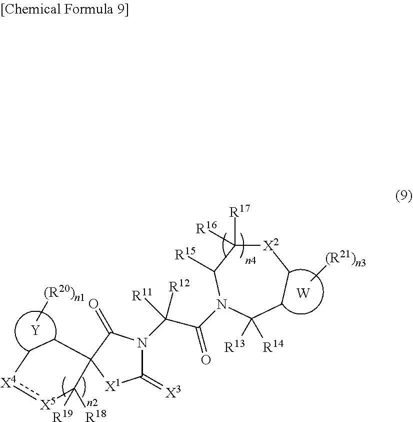
wherein
X¹ is independently —O—, —NR¹—, or —S—;
R¹ is independently a hydrogen atom, C₁₋₆ alkyl, or C₃₋₆ cycloalkyl;
X² is independently —C(R²)(R³)—, —O—, —N(R⁴)—, or —S(O)ₙ₁—;
R² and R³ are each independently a hydrogen atom, a deuterium atom, C₁₋₆ alkyl, C₁₋₆ haloalkyl, or C₃₋₆ cycloalkyl;
R⁴ is each independently a hydrogen atom, C₁₋₆ alkyl, C₃₋₆ cycloalkyl, —C(═O)(C₁₋₆ alkyl), —S(O)₂(C₁₋₆ alkyl), —C(═O)(C₃₋₆ cycloalkyl), or —S(O)₂(C₃₋₆ cycloalkyl);
X³ is each independently O or NH; [symbol]
[Chemical Formula 10]
is a single bond or a double bond;
wherein if [symbol]
[Chemical Formula 11]
is a single bond, X⁴ is independently —C(R⁵)(R⁶)—, —O—, —C(═O)—, —NR⁷—, or —S(O)ₙ₁—;
wherein if [symbol]
[Chemical Formula 12]
is a single bond, X⁵ is independently —C(R⁸)(R⁹)—, —O—, —C(═O)—, —NR¹⁰—, —S(O)ₙ₁—, or a direct bond;
wherein if [symbol]
[Chemical Formula 13]
is a double bond, X⁴ is independently —C(R⁵)—;
wherein if [symbol]
[Chemical Formula 14]
is a double bond, X⁵ is independently —C(R⁸)—;
R⁵ and R⁶ are each independently a hydrogen atom, OH, a halogen atom, CN, C₁₋₆ alkyl, C₁₋₆ haloalkyl, C₃₋₆ cycloalkyl, or C₁₋₆ alkoxy;
R⁸ and R⁹ are each independently a hydrogen atom, OH, a halogen atom, or C₁₋₆ alkyl;
R⁷ is each independently a hydrogen atom, C₁₋₆ alkyl, or C₃₋₆ cycloalkyl;
R¹⁰ is each independently a hydrogen atom, C₁₋₆ alkyl, or C₃₋₆ cycloalkyl;
Y is independently a C₆-10 aromatic ring or a C₅₋₁₀ heteroaromatic ring (wherein the group is each independently unsubstituted, or optionally substituted with 1 to 2 R²⁰);
R¹¹ and R¹² are each independently a hydrogen atom, a deuterium atom, C₁₋₆ alkyl, C₁₋₆ haloalkyl, or C₃₋₆ cycloalkyl;
R¹³ and R¹⁴ are each independently a hydrogen atom, a deuterium atom, C₁₋₆ alkyl, C₁₋₆ haloalkyl, or C₃₋₆ cycloalkyl;
R¹⁶ and R¹⁷ are each independently a hydrogen atom, a deuterium atom, C₁₋₆ alkyl, C₁₋₆ haloalkyl, or C₃₋₆ cycloalkyl;
R¹⁸ and R¹⁹ are each independently a hydrogen atom, a halogen atom, or C₁₋₆ alkyl;
R¹⁵ is each independently a hydrogen atom, C₁₋₆ alkyl substituted with 0 to 2 R^{a}, C₁₋₆ haloalkyl, or M^{a};
wherein R^{a} is independently C₁₋₆ alkyl, C₂₋₆ alkenyl, C₂₋₆ alkynyl, a halogen atom, C₁₋₆ haloalkyl, C₁₋₆ haloalkoxy, —CN, hydroxyl, —OM^{e}, —SM^{e}, —S(O)₂M^{e}, —C(O)NM^{f}M^{g}, —NM^{f}M^{g}, —N(M^{e})C(O)M^{h}, —N(M^{e})S(O) M, —N(M^{e})C(O)OM, —N(M^{e})C(O)NM^{f}M^{g}, or M^{b};
R²⁰ is independently a hydrogen atom, a halogen atom, —OH, —CN, —COOH, C₁₋₆ alkyl, C₁₋₆ alkoxy, C₁₋₆ haloalkoxy, C₂₋₁₀ alkoxyalkyl, C₄₋₂₀ alkoxyalkylalkynyl, C₂₋₁₀ haloalkoxyalkyl, C₁₋₆ hydroxyalkyl, C₃₋₁₀ hydroxyalkylalkynyl, C₂₋₁₀ hydroxyalkynyl, —B(R^{b}) (R^{d}), —S(O)ₙ₁R^{c}, —N(R^{c})₂, —C(═O)N(R^{c})—, —NHC(═O)R^{c}, —NHC(═O)OR^{c}, —NHC(═O)C(═O)N(R^{c})₂, —NHC(═O)C(═O)OR^{c}, —NHC(═O)N(R^{c})₂, —NHC(═O)NR^{c}C(═O)N(R^{c})₂, —NHC(═O)NR^{c}S(O)₂OR^{c}, —NHC(═O)NR^{c}S(O)₂N(R^{c})₂, —NHC(═S)N(R^{c})₂, —NHC(═NC≡N)NR^{c}, —NHC(═NC≡N)SR^{c}, —NHS(O)ₙ₁R^{c}, M^{c}, —(C₁₋₆ alkylene)-B(R^{b})(R^{d}), —(C₁₋₆ alkylene)-S(O)ₙ₁R^{c}, —(C₁₋₆ alkylene)-N(R^{c})₂, —(C₁₋₆ alkylene)-C(═O)N(R^{c})₂, —(C₁₋₆ alkylene)-NHC(═O)R^{c}, —(C₁₋₆ alkylene)-NHC(═O)OR^{c}, —(C₁₋₆ alkylene)-NHC(═O)C(═O)N(R^{c})₂, —(C₁₋₆ alkylene)-NHC(═O)C(═O)OR^{c}, —(C₁₋₆ alkylene)-NHC(═O)N(R^{c})₂, —(C₁₋₆ alkylene)-NHC(═O)NR^{c}C(═O)N(R^{c})₂, —(C₁₋₆ alkylene)-NHC(═O)NR^{c}S(O)₂OR^{c}, —(C₁₋₆ alkylene)-NHC(═O)NRCS(O)₂N(R^{c})₂, —(C₁₋₆ alkylene)-NHC(═S)N(R^{c})₂, —(C₁₋₆ alkylene)-NHC(═NC≡N)NR^{c}, —(C₁₋₆ alkylene)-NHC(═NC≡N)SR^{c}, —(C₁₋₆ alkylene)-NHS(O)ₙ₁R^{c}, —(C₁₋₆ alkylene)-M^{c}, —CH≡CH—(C₁₋₆ alkyl), —CH═CH-M^{c}, —OM^{c}, —SM^{c}, or —N(R^{c})M^{c};
R^{b} and R^{d} are each independently a hydrogen atom, hydroxyl, or C₁₋₆ alkyl;
R^{c} is each independently a hydrogen atom, C₁₋₆ alkyl, C₆-10 aryl, 5- to 10-membered cyclic heteroaryl, a 3- to 10-membered cyclic non-aromatic heterocyclic group, C₃₋₁₀ cycloalkyl, or C₅₋₁₀ cycloalkenyl (wherein the group is each independently not substituted, or optionally substituted with 1 to 2 substituents selected from amino, hydroxy, methoxy, C₁₋₆ alkyl, C₃₋₁₀ cycloalkyl, or CN);
M^{a}, M^{b}, and M^{c} are each independently C₆₋₁₀ aryl, C₅₋₁₀ heteroaryl, a C₃₋₁₀ non-aromatic heterocyclic group, C₃₋₁₀ cycloalkyl, or C₃₋₁₀ cycloalkenyl (wherein the groups are each independently not substituted, or optionally substituted with 1 to 2 M^{d});
M^{d} is each independently C₁₋₆ alkyl, C₂₋₆ alkenyl, C₂₋₆ alkynyl, halogen, C₁₋₆ haloalkyl, —CN, oxo, —OM, —OC(O)M^{h}, —OC(O)NM^{f}M^{g}, —SM^{e}, —S(O)₂M^{e}, —S(O)₂NM^{f}M^{g}, —C(O)M^{e}, —C(O)-5- to 10-membered cyclic monocyclic cycloheteroaryl, —C(O)-5- to 10-membered cyclic monocyclic heteroaryl, —C(O)OM^{e}, —C(O)NM^{f}M^{g}, —NM^{f}M^{g}, —N(M^{e})C(O)M^{h}, —N(M^{e})S(O)M^{h}, —N(M^{e})C(O)OM^{h}, —N(M^{e})C(O)NM^{f}M^{g}, —(C₁₋₆ alkylene)OM^{e}, —(C₁₋₆ alkylene)-OC(O)M^{h}, —(C₁₋₆ alkylene)-OC(O)NM^{f}M^{g}, —(C₁₋₆ alkylene)-S(O)₂M^{e}, —(C₁₋₆ alkylene)-S(O)₂NM^{f}M^{g}, —(C₁₋₆ alkylene)-C(O)M, —(C₁₋₆ alkylene)-C(O)OM^{e}, —(C₁₋₆ alkylene)-C(O)NM^{f}M^{g}, —(C₁₋₆ alkylene)-NM^{f}M^{g}, —(C₁₋₆ alkylene)-N(M^{e})C(O)M^{h}, —(C₁₋₆ alkylene)-N(M^{e})S(O)₂M^{h}, —(C₁₋₆ alkylene)-N(M^{e})C(O)OM^{h}, —(C₁₋₆ alkylene)-N(M)C(O)NM^{f}M^{g}, or (C₁₋₆ alkylene)-CN;
W is independently a C₆₋₁₀ aromatic ring or a C₅₋₁₀ heteroaromatic ring (wherein the group is independently not substituted, or optionally substituted with 1 to 3 R²¹);
R²¹ is each independently C₁₋₆ alkyl, a halogen atom, C₁₋₆ haloalkyl, C₁₋₆ haloalkoxy, C₃₋₆ cycloalkyl, —OM^{e}, —OC(O)M^{h}, —OC(O)NM^{f}M^{g}, —SM^{e}, —S(O)₂M^{e}, —S(O)₂NM^{f}M^{g}, —C(O)M^{e}, —C(O)OM^{e}, —C(O)NM^{f}M^{g}, —N(M^{e})C(O)M^{h}, —N(M^{e})S(O)₂M^{h}, —N(M^{e})C(O)OM^{h}, or —N(M^{e})C(O)NM^{f}M^{g};
M^{c}, M^{f}, and M^{g} are each independently a hydrogen atom, C₁₋₆ alkyl, C₁₋₆ haloalkyl, or C₃₋₆ cycloalkyl;
M^{h} is each independently C₁₋₆ alkyl, C₁₋₆ haloalkyl, or C₃₋₆ cycloalkyl;
n1 and n2 are, for each instance, independently 0, 1, or 2; and
n3 and n4 are, for each instance, independently 0, 1, 2, or 3,
or a prodrug thereof or a pharmaceutically acceptable salt thereof.

49. The pharmaceutical composition of claim 31, wherein the low molecular weight compound is a compound represented by the following (Table 9)
TABLE 9 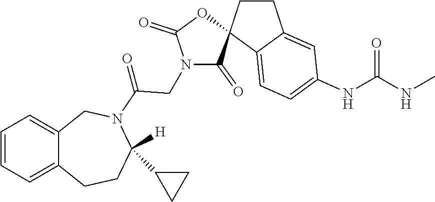 [SMILES: CNC(=O)Nc1ccc2c(c1)CC[C@@]21OC(=O)N(CC(=O)N2Cc3ccccc3CC[C@H]2C2CC2)C1=O] 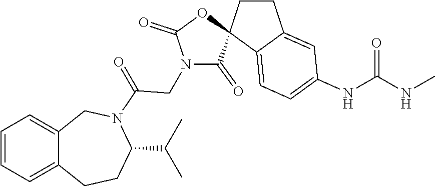 [SMILES: CNC(=O)Nc1ccc2c(c1)CC[C@]21OC(=O)N(CC(=O)N2Cc3ccccc3CC[C@H]2C(C)C)C1=O] 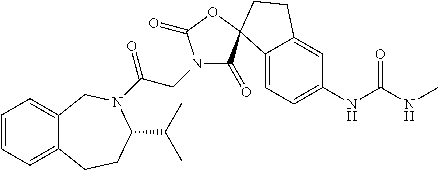 [SMILES: CNC(=O)Nc1ccc2c(c1)CC[C@@]21OC(=O)N(CC(=O)N2Cc3ccccc3CC[C@H]2C(C)C)C1=O] 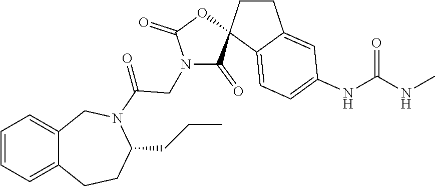 [SMILES: CCC[C@@H]1CCc2ccccc2CN1C(=O)CN1C(=O)O[C@@]2(CCc3cc(NC(=O)NC)ccc32)C1=O] 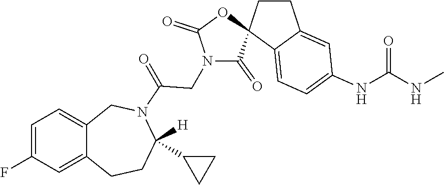 [SMILES: CNC(=O)Nc1ccc2c(c1)CC[C@]21OC(=O)N(CC(=O)N2Cc3ccc(F)cc3CC[C@H]2C2CC2)C1=O] 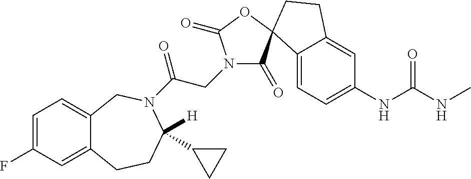 [SMILES: CNC(=O)Nc1ccc2c(c1)CC[C@@]21OC(=O)N(CC(=O)N2Cc3ccc(F)cc3CC[C@H]2C2CC2)C1=O] 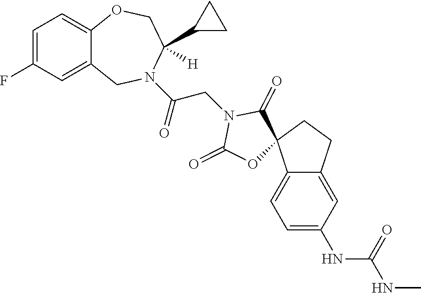 [SMILES: CNC(=O)Nc1ccc2c(c1)CC[C@@]21OC(=O)N(CC(=O)N2Cc3cc(F)ccc3OC[C@H]2C2CC2)C1=O] 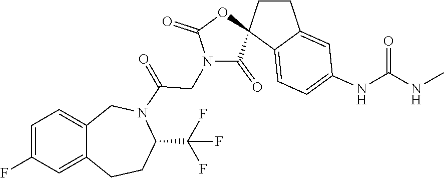 [SMILES: CNC(=O)Nc1ccc2c(c1)CC[C@]21OC(=O)N(CC(=O)N2Cc3ccc(F)cc3CC[C@H]2C(F)(F)F)C1=O] 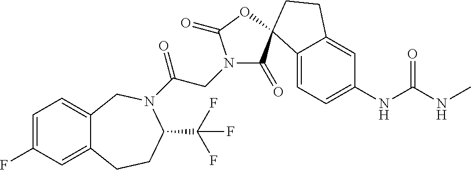 [SMILES: CNC(=O)Nc1ccc2c(c1)CC[C@@]21OC(=O)N(CC(=O)N2Cc3ccc(F)cc3CC[C@H]2C(F)(F)F)C1=O] 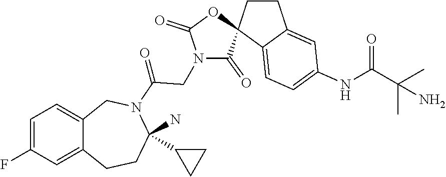 [SMILES: CC(C)(N)C(=O)Nc1ccc2c(c1)CC[C@]21OC(=O)N(CC(=O)N2Cc3ccc(F)cc3CC[C@@]2(N)C2CC2)C1=O] 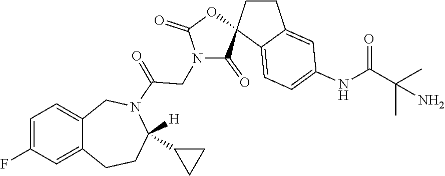 [SMILES: CC(C)(N)C(=O)Nc1ccc2c(c1)CC[C@@]21OC(=O)N(CC(=O)N2Cc3ccc(F)cc3CC[C@H]2C2CC2)C1=O] 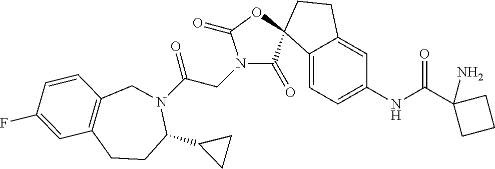 [SMILES: NC1(C(=O)Nc2ccc3c(c2)CC[C@]32OC(=O)N(CC(=O)N3Cc4ccc(F)cc4CC[C@H]3C3CC3)C2=O)CCC1] 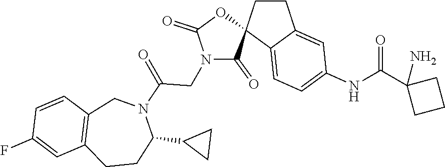 [SMILES: NC1(C(=O)Nc2ccc3c(c2)CC[C@@]32OC(=O)N(CC(=O)N3Cc4ccc(F)cc4CC[C@H]3C3CC3)C2=O)CCC1] 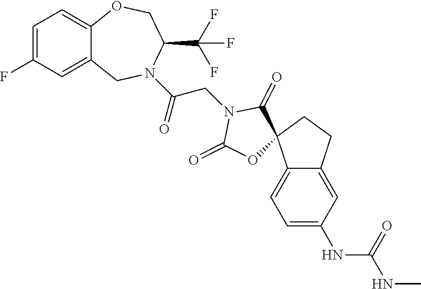 [SMILES: CNC(=O)Nc1ccc2c(c1)CC[C@@]21OC(=O)N(CC(=O)N2Cc3cc(F)ccc3OC[C@H]2C(F)(F)F)C1=O] 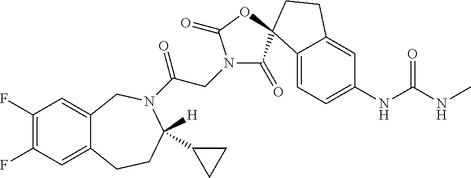 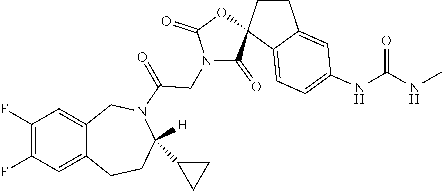 [SMILES: CNC(=O)Nc1ccc2c(c1)CC[C@@]21OC(=O)N(CC(=O)N2Cc3cc(F)c(F)cc3CC[C@H]2C2CC2)C1=O] 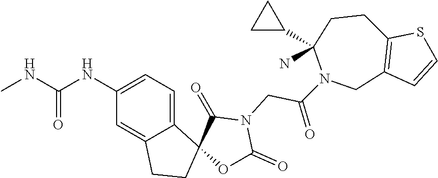 [SMILES: CNC(=O)Nc1ccc2c(c1)CC[C@@]21OC(=O)N(CC(=O)N2Cc3ccsc3CC[C@@]2(N)C2CC2)C1=O] 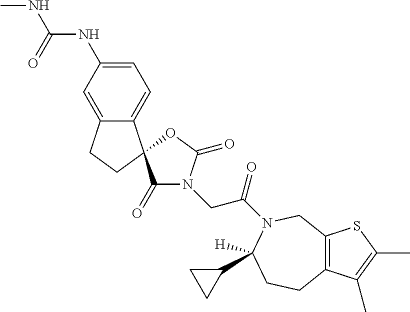 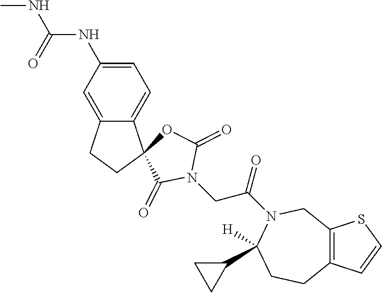 [SMILES: CNC(=O)Nc1ccc2c(c1)CC[C@]21OC(=O)N(CC(=O)N2Cc3sccc3CC[C@H]2C2CC2)C1=O] 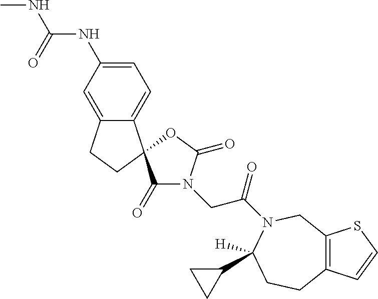 [SMILES: CNC(=O)Nc1ccc2c(c1)CC[C@@]21OC(=O)N(CC(=O)N2Cc3sccc3CC[C@H]2C2CC2)C1=O] 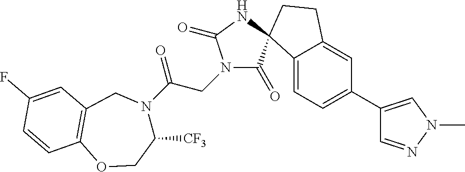 [SMILES: Cn1cc(-c2ccc3c(c2)CC[C@]32NC(=O)N(CC(=O)N3Cc4cc(F)ccc4OC[C@H]3C(F)(F)F)C2=O)cn1] 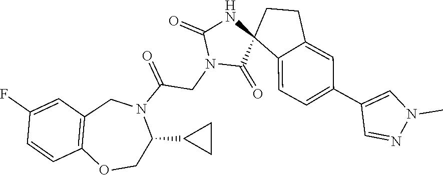 [SMILES: Cn1cc(-c2ccc3c(c2)CC[C@]32NC(=O)N(CC(=O)N3Cc4cc(F)ccc4OC[C@H]3C3CC3)C2=O)cn1] 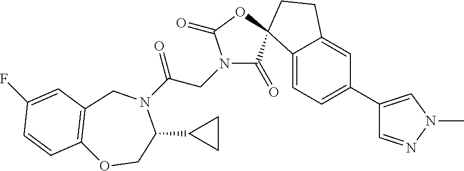 [SMILES: Cn1cc(-c2ccc3c(c2)CC[C@]32OC(=O)N(CC(=O)N3Cc4cc(F)ccc4OC[C@H]3C3CC3)C2=O)cn1] 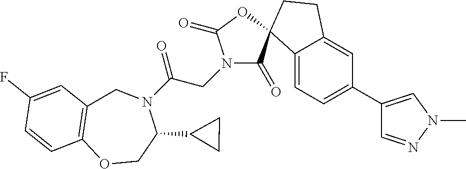 [SMILES: Cn1cc(-c2ccc3c(c2)CC[C@@]32OC(=O)N(CC(=O)N3Cc4cc(F)ccc4OC[C@H]3C3CC3)C2=O)cn1] 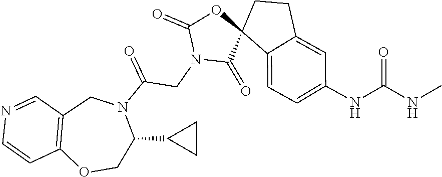 [SMILES: CNC(=O)Nc1ccc2c(c1)CC[C@]21OC(=O)N(CC(=O)N2Cc3cnccc3OC[C@H]2C2CC2)C1=O]
or a pharmaceutically acceptable salt thereof.

50. The pharmaceutical composition of claim 31, wherein the low molecular weight compound is a compound represented by formula (10) 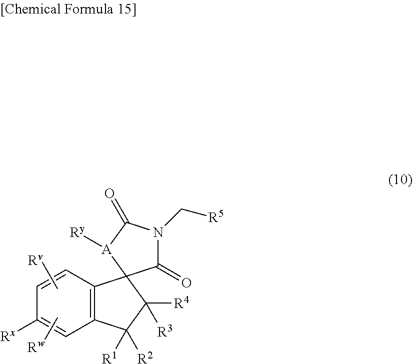
wherein
A is independently selected from O, N, and S;
R^{y} is absent, a hydrogen atom, alkyl, substituted alkyl, or alkenyl;
R^{y}, R^{w}, and R^{x} are each independently a hydrogen atom, a halogen atom, cyano, nitro, alkyl, substituted alkyl, alkenyl, alkynyl, cycloalkyl, substituted cycloalkyl, a heterocycle, a substituted heterocycle, aryl, substituted aryl, an aromatic heterocycle, a substituted aromatic heterocycle, substituted amide, substituted guanidino, substituted urea, amino, substituted amino, alkoxy, or substituted alkoxy;
R¹, R², R³, and R⁴ are each independently a hydrogen atom, alkyl, or a halogen atom;
wherein R¹ and R², R² and R³, or R³ and R⁴ together may from a ring;
R⁵ is alkyl, alkoxy, amino, substituted amino, amide, substituted amide, ester, carbonyl, a heterocycle, or a substituted heterocycle,
or a prodrug thereof or a pharmaceutically acceptable salt thereof.

51. The pharmaceutical composition of claim 31, wherein the low molecular weight compound is a compound represented by the following (Table 10)
TABLE 10 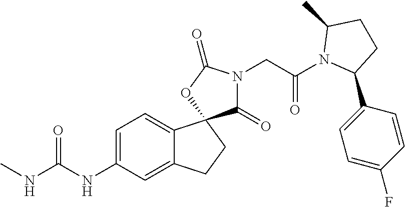 [SMILES: CNC(=O)Nc1ccc2c(c1)CC[C@@]21OC(=O)N(CC(=O)N2[C@@H](C)CC[C@H]2c2ccc(F)cc2)C1=O] 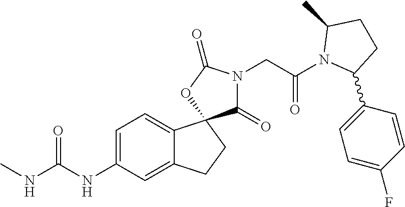 [SMILES: CNC(=O)Nc1ccc2c(c1)CC[C@@]21OC(=O)N(CC(=O)N2C(c3ccc(F)cc3)CC[C@@H]2C)C1=O] 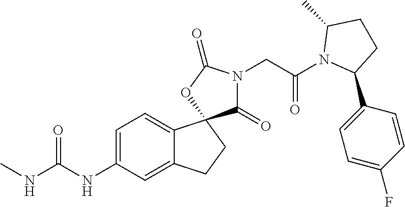 [SMILES: CNC(=O)Nc1ccc2c(c1)CC[C@@]21OC(=O)N(CC(=O)N2[C@H](C)CC[C@H]2c2ccc(F)cc2)C1=O] 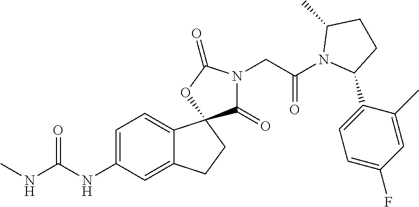 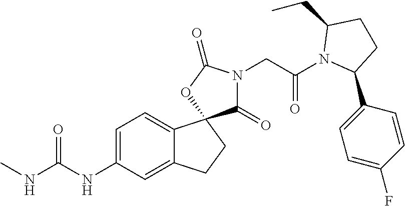 [SMILES: CC[C@H]1CC[C@@H](c2ccc(F)cc2)N1C(=O)CN1C(=O)O[C@@]2(CCc3cc(NC(=O)NC)ccc32)C1=O] 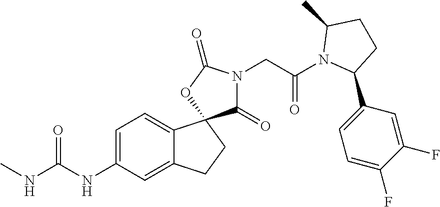 [SMILES: CNC(=O)Nc1ccc2c(c1)CC[C@@]21OC(=O)N(CC(=O)N2[C@@H](C)CC[C@H]2c2ccc(F)c(F)c2)C1=O] 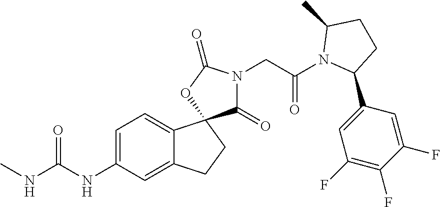 [SMILES: CNC(=O)Nc1ccc2c(c1)CC[C@@]21OC(=O)N(CC(=O)N2[C@@H](C)CC[C@H]2c2cc(F)c(F)c(F)c2)C1=O] 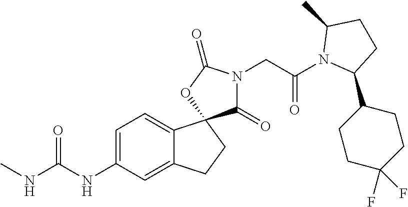 [SMILES: CNC(=O)Nc1ccc2c(c1)CC[C@@]21OC(=O)N(CC(=O)N2[C@@H](C)CC[C@H]2C2CCC(F)(F)CC2)C1=O] 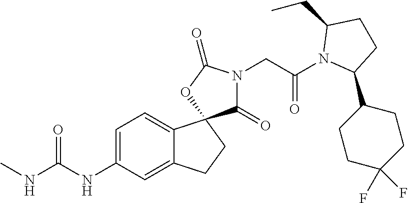 [SMILES: CC[C@H]1CC[C@@H](C2CCC(F)(F)CC2)N1C(=O)CN1C(=O)O[C@@]2(CCc3cc(NC(=O)NC)ccc32)C1=O] 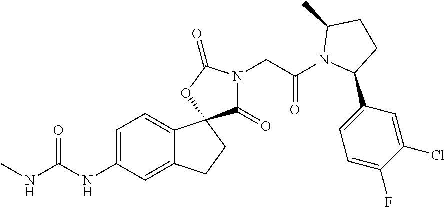 [SMILES: CNC(=O)Nc1ccc2c(c1)CC[C@@]21OC(=O)N(CC(=O)N2[C@@H](C)CC[C@H]2c2ccc(F)c(Cl)c2)C1=O] 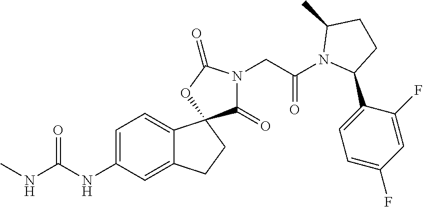 [SMILES: CNC(=O)Nc1ccc2c(c1)CC[C@@]21OC(=O)N(CC(=O)N2[C@@H](C)CC[C@H]2c2ccc(F)cc2F)C1=O] 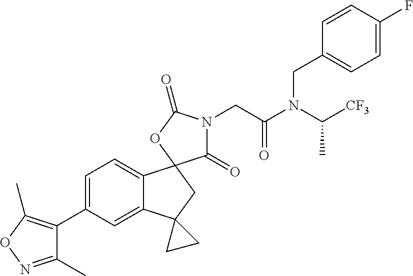 [SMILES: Cc1noc(C)c1-c1ccc2c(c1)C1(CC1)CC21OC(=O)N(CC(=O)N(Cc2ccc(F)cc2)[C@@H](C)C(F)(F)F)C1=O] 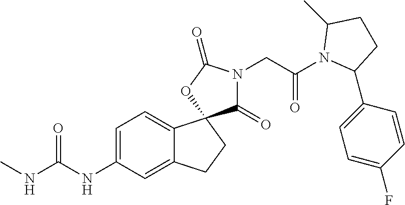 [SMILES: CNC(=O)Nc1ccc2c(c1)CC[C@@]21OC(=O)N(CC(=O)N2C(C)CCC2c2ccc(F)cc2)C1=O] 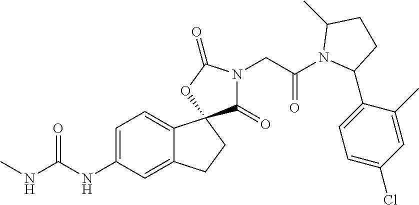 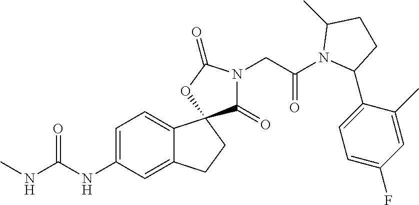 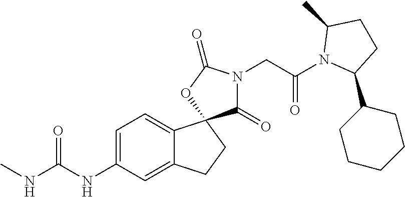 [SMILES: CNC(=O)Nc1ccc2c(c1)CC[C@@]21OC(=O)N(CC(=O)N2[C@@H](C)CC[C@H]2C2CCCCC2)C1=O] 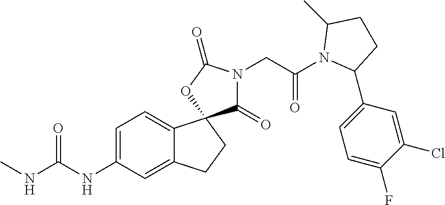 [SMILES: CNC(=O)Nc1ccc2c(c1)CC[C@@]21OC(=O)N(CC(=O)N2C(C)CCC2c2ccc(F)c(Cl)c2)C1=O] 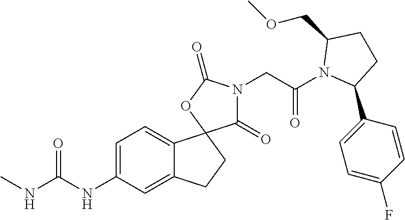 [SMILES: CNC(=O)Nc1ccc2c(c1)CCC21OC(=O)N(CC(=O)N2[C@@H](COC)CC[C@H]2c2ccc(F)cc2)C1=O] 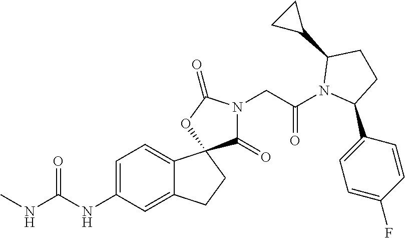 [SMILES: CNC(=O)Nc1ccc2c(c1)CC[C@@]21OC(=O)N(CC(=O)N2[C@@H](C3CC3)CC[C@H]2c2ccc(F)cc2)C1=O] 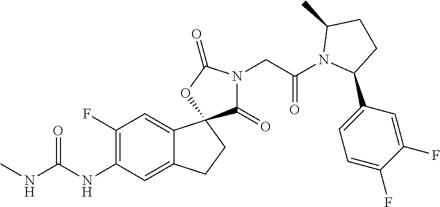 [SMILES: CNC(=O)Nc1cc2c(cc1F)[C@@]1(CC2)OC(=O)N(CC(=O)N2[C@@H](C)CC[C@H]2c2ccc(F)c(F)c2)C1=O] 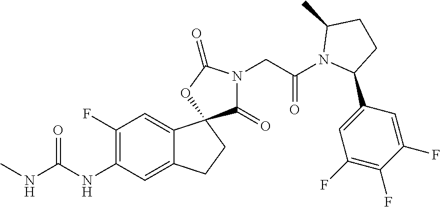 [SMILES: CNC(=O)Nc1cc2c(cc1F)[C@@]1(CC2)OC(=O)N(CC(=O)N2[C@@H](C)CC[C@H]2c2cc(F)c(F)c(F)c2)C1=O]
or a pharmaceutically acceptable salt thereof.

52. The pharmaceutical composition of claim 31, wherein the low molecular weight compound is a compound represented by formula (11) 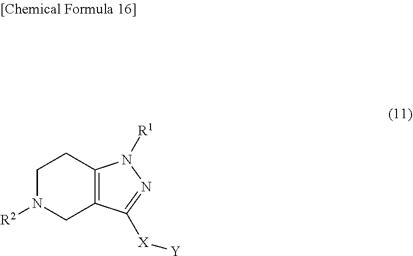
wherein
R¹ is C₁₋₁₂ alkyl, C₂₋₁₂ alkenyl, C₂₋₁₂ alkynyl, 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R¹ is optionally substituted with one or more R^{d};
R² is —C(O)—N(R^{e})₂, —S(O)—N(R^{e})₂, —S(O)₂—N(R^{e})₂, —C(O)—R, —C(O)—O—(R^{c}), —S(O)—R^{c}, or —S(O)₂—R^{c};
X is absent, —C(O), or C₁₋₃ alkyl;
Y is phenyl, a 9-membered bicyclic carbocyclic ring, a 10-membered bicyclic carbocyclic ring, a 9-membered bicyclic heterocycle, or a 10-membered bicyclic heterocycle;
wherein Y is optionally substituted with R^{a}, and Y is optionally further substituted with one or more R^{b}; or
X combined with Y is selected from the group consisting of 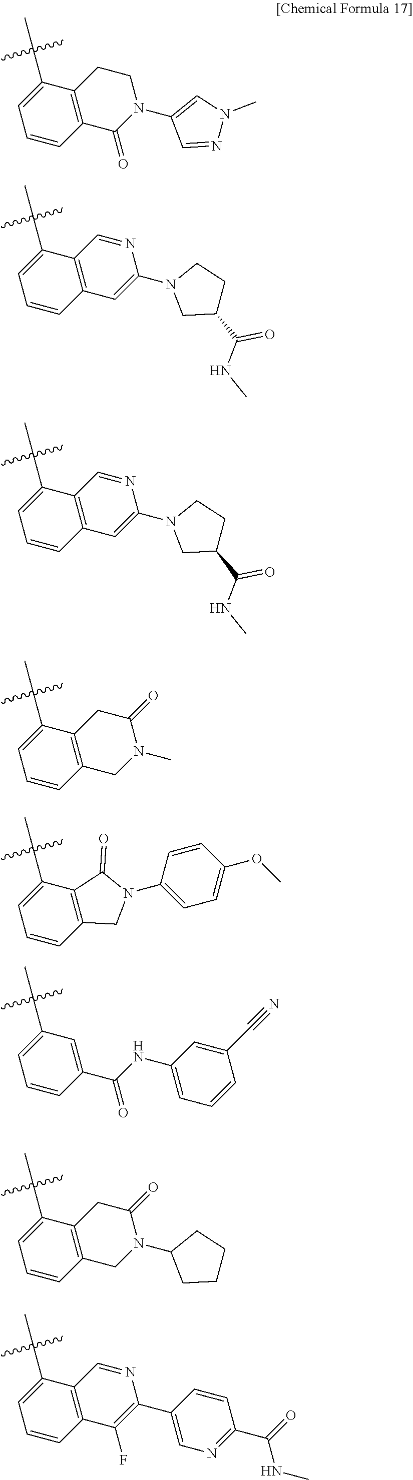 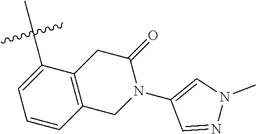 [SMILES: *c1cccc2c1CC(=O)N(c1cnn(C)c1)C2] 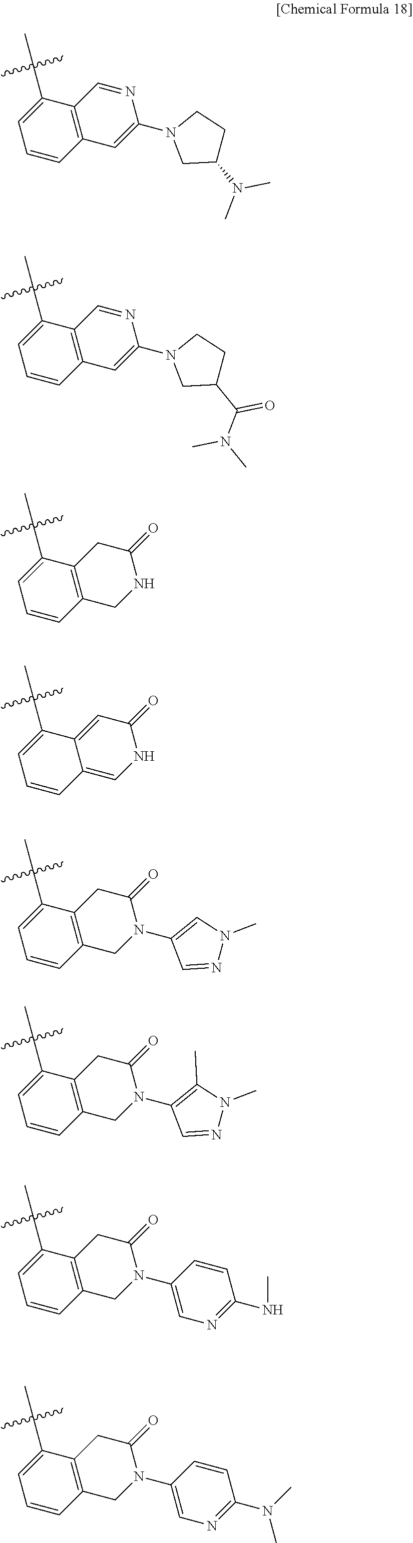 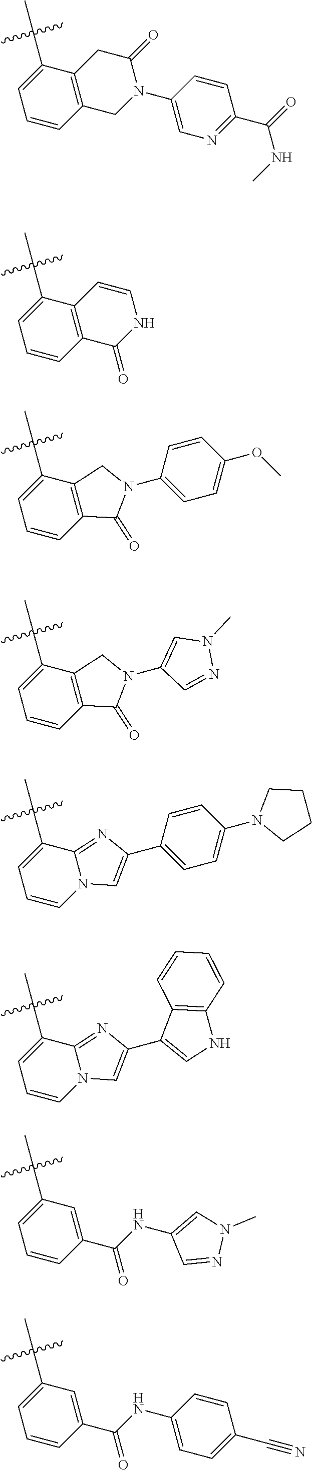 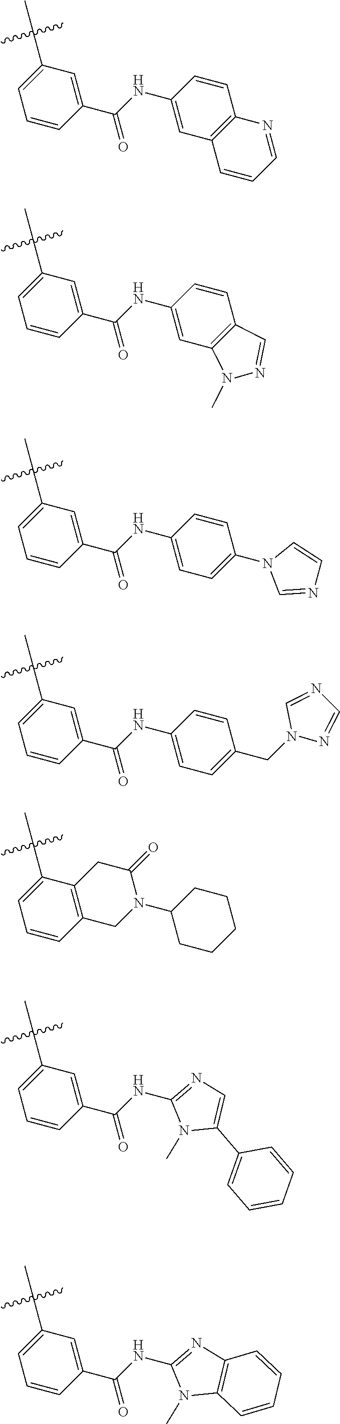 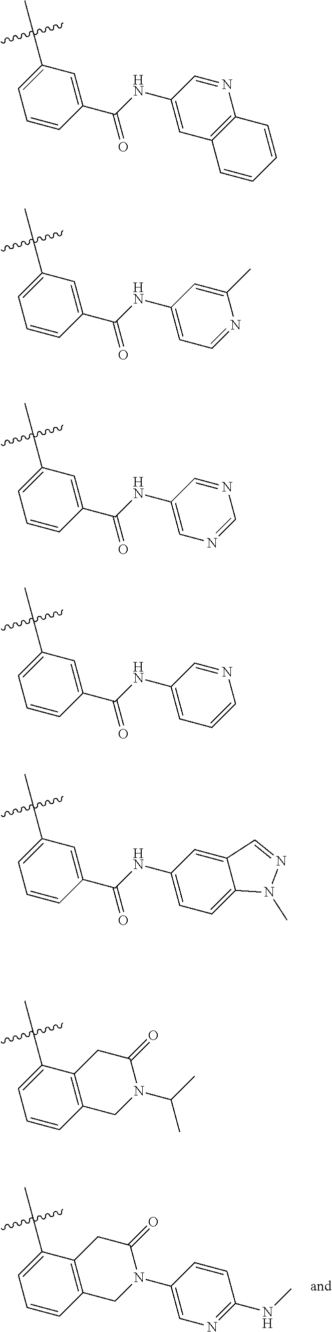 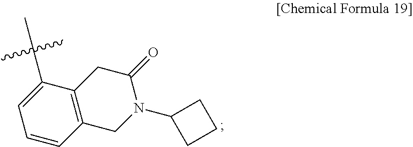 [SMILES: *c1cccc2c1CC(=O)N(C1CCC1)C2]
each R^{a} is independently selected from the group consisting of a 5-membered carbocyclic ring, a 6-membered carbocyclic ring, a 5-membered heterocycle, and a 6-membered heterocycle, wherein the 5-membered carbocyclic ring, 6-membered carbocyclic ring, 5-membered heterocycle, and 6-membered heterocycle are optionally substituted with one or more R^{c};
each R^{b} is independently selected from the group consisting of a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarboryl, C₁₋₄ alkanoyl, —C(O)—N(R^{f})₂, —N(R^{f})C(O)—R^{f}, and C₁₋₄ alkanoyloxy, wherein each of C₂₋₄ alkyl, C₂₋₄ alkenyl, C₁₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₄ alkoxy, and C₁₋₃ alkyl that is optionally substituted with one or more groups independently selected from halogen;
R^{c} is independently selected from the group consisting of a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy, wherein each of C₁₋₄ alkyl, C₂₋₄ alkenyl, C₂₋₄ alkynyl, C₂₋₆ cycloalkyl, (C₂₋₆ cycloalkyl) C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₃ alkoxy, C₁₋₃ alkyl, and C₁₋₃ alkyl that is optionally substituted with one or more groups independently selected from halogen;
each R^{d} is independently selected from the group consisting of oxo, a halogen atom, cyano, a hydroxyl group, amino, C₁₋₄ alkyl, C₂₋₄ alkenyl, C₂₋₄ alkynyl, C₂₋₆ cycloalkyl, (C₂₋₆ cycloalkyl)C₁₋₄ alkyl, C₁₋₄ alkoxy, C₁₋₄ alkoxycarbonyl, C₁₋₄ alkanoyl, and C₁₋₄ alkanoyloxy, wherein each of C₁₋₄ alkyl, C₂₋₄ alkenyl, C₂₋₄ alkynyl, C₂₋₆ cycloalkyl, (C₂₋₆ cycloalkyl) C₁₋₄ alkyl, C₁₋₄ alkoxy, C₁₋₄ alkoxycarbonyl, C₁₋₄ alkanoyl, and C₁₋₄ alkanoyloxy is optionally substituted with one or more groups independently selected from oxo, halogen, amino, a hydroxyl group, C₁₋₃ alkoxy, and C₁₋₃ alkyl that is optionally substituted with one or more groups independently selected from halogen;
each R^{e} is independently selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkyl, and C₁₋₃ alkyl that is optionally substituted with one or more groups independently selected from halogen;
each R^{f} is a hydrogen atom or C₁₋₄ alkyl; or
selected from the group consisting of 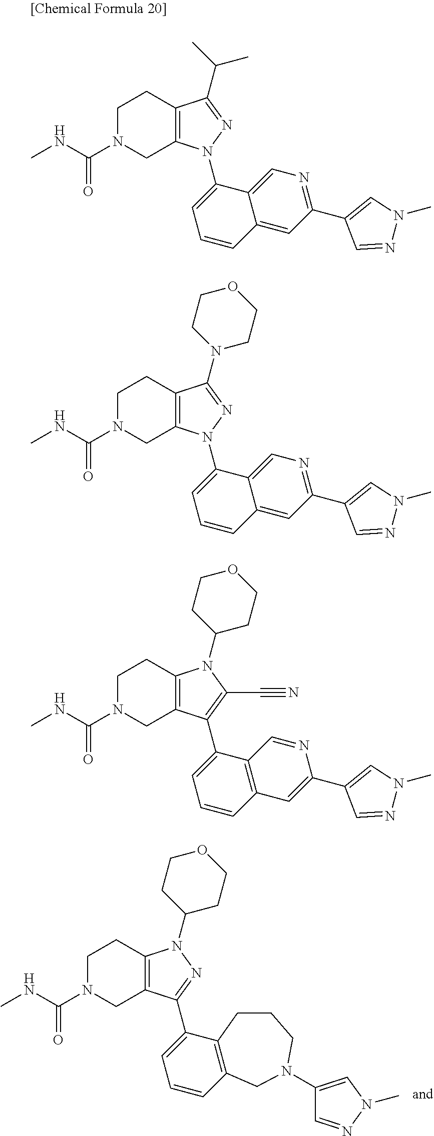 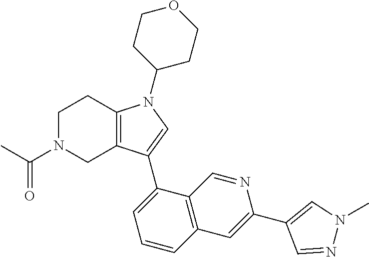 [SMILES: CC(=O)N1CCc2c(c(-c3cccc4cc(-c5cnn(C)c5)ncc34)cn2C2CCOCC2)C1]
or a prodrug thereof or a pharmaceutically acceptable salt thereof.

53. The pharmaceutical composition of claim 31, wherein the low molecular weight compound is a compound represented by the following (Table 11)
TABLE 11 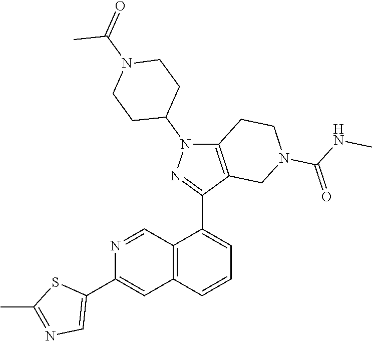 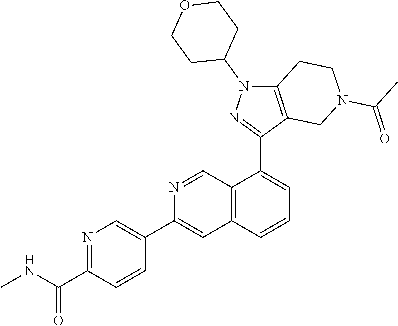 [SMILES: CNC(=O)c1ccc(-c2cc3cccc(-c4nn(C5CCOCC5)c5c4CN(C(C)=O)CC5)c3cn2)cn1] 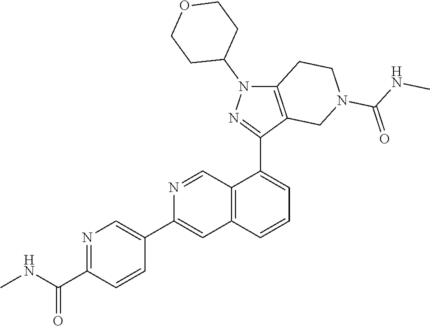 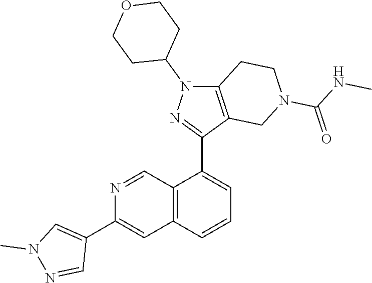
or a pharmaceutically acceptable salt thereof.

54. The pharmaceutical composition of claim 31, wherein the low molecular weight compound is a compound represented by formula (12) or (13) 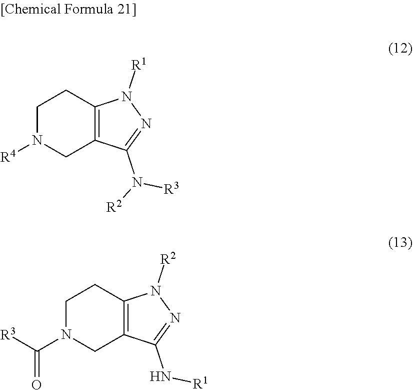
wherein
R¹ in formula (14) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R¹ is optionally substituted with one or more R^{b};
R² in formula (14) is selected from C₆₋₂₀ aryl, C₁₋₂₀ heteroaryl, —(C₆₋₂₀ aryl)-(C₁₋₂₀ heteroaryl), —(C₁₋₂₀ heteroaryl)-(C₆₋₂₀ aryl), and —(C₁₋₂₀ heteroaryl)-(C₁₋₂₀ heteroaryl), wherein each of C₆₋₂₀ aryl, C₁₋₂₀ heteroaryl, —(C₆₋₂₀ aryl)-(C₁₋₂₀ heteroaryl), and (C₁₋₂₀ heteroaryl)-(C₁₋₂₀ heteroaryl) is independently optionally substituted with one or more substituents independently selected from R^{c}, oxo, fluorine, chlorine, bromine, iodine, —NO₂, —N(R^{a})₂, —CN, —C(O)—N(R^{a})₂, —S(O)—N(R^{a})₂, —S(O)₂—N(R^{a})₂, —O—R^{a}, —S—R^{a}, —O—C(O)—R^{a}, —O—C(O)—O—R^{a}, —C(O)—R^{a}, —C(O)—O—R^{a}, —S(O)—R^{a}, —S(O)₂—R^{a}, —O—C(O)—N(R^{a})₂, —N(R^{a})—C(O)—OR^{a}, —N(R^{a})—C(O)—N(R^{a})₂, —N(R^{a})—C(O)—R^{a}, —N(R^{a})—S(O)—R^{a}, —N(R^{a})—S(O)₂—R^{a}, —N(R^{a})—S(O)—N(R^{a})₂, and —N(R^{a})—S(O)₂—N(R^{a})₂;
R³ in formula (14) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R³ is optionally substituted with one or more R^{e}; or
R² and R³ in formula (14), together with the nitrogen to which they are attached, form a 3- to 12-membered heterocycle optionally substituted with one or more R^{c};
R⁴ in formula (14) is C₁₋₄ alkyl, C₂₋₄ alkenyl, C₂₋₄ alkynyl, a 3- to 5-membered carbocyclic ring, a 3- to 5-membered heterocycle, —C(O)—N(R^{h})—, —S(O)—N(R^{h})—, —S(O)₂—N(R^{h})₂, —C(O)—R^{h}, —C(O)—OR^{h}, —S(O)—R^{h}, or —S(O)₂—R^{a}, wherein any of C₁₋₄ alkyl, C₂₋₄ alkenyl, C₂₋₄ alkynyl, 3- to 5-membered carbocyclic ring, and 3- to 5-membered heterocycle is optionally substituted with one or more substituents independently selected from fluorine, chlorine, bromine, iodine, a 3- to 5-membered carbocyclic ring, —C(O)—N(R^{h}), —S(O)—N(R^{h})₂, —S(O)₂—N(R^{h})₂, —O—R^{h}, —S—R^{h}, —O—C(O)—R^{h}, —O—C(O)—O—R^{h}, —C(O)—R^{h}, —C(O)—O—R^{h}, —S(O)—R^{h}, —S(O)₂—R^{h}, —O—C(O)—N(R^{h})₂, —N(R^{h})—C(O)—OR^{h}, —N(R^{h})—C(O)—N(R^{h})₂, —N(R^{h})—C(O)—R^{h}, —N(R^{h})—S(O)—R^{h}, —N(R^{h})—S(O)₂—R^{h}, —N(R^{h})—S(O)—N(R^{h})₂, and —N(R^{h})—S(O)₂—N(R^{h})₂;
each R^{a} in formula (14) is independently selected from a hydrogen atom, C₁₋₆ alkyl, C₂₋₆ alkenyl, C₂₋₆ alkynyl, a carbocyclic ring, and a heterocycle, wherein each of C₁₋₆ alkyl, C₂₋₆ alkenyl, C₂₋₆ alkynyl, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{a}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{b} in formula (14) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —O—C(O)—O—R^{c}, —C(O)—R^{c}, —C(O)—O—R^{c}, —S(O)—R^{c}, —S(O)₂—R^{c}, —O—C(O)—N(R^{c})₂, —N(R^{c})—C(O)—OR^{c}, —N(R^{c})—C(O)—N(R^{c})₂, —N(R^{c})—C(O)—R^{c}, —N(R^{c})—S(O)—R^{c}, —N(R^{c})—S(O)₂—R^{c}, —N(R^{c})—S(O)—N(R^{c})₂, and —N(R^{c})—S(O)₂—N(R^{c})₂, wherein any of C₁₋₆ alkyl, C₂₋₆ alkenyl, C₂₋₆ alkynyl, carbocyclic ring, heterocycle, aryl, and heteroaryl is optionally substituted with one or more groups independently selected from oxo, halogen, —NO₂, —N(R^{c})₂, —CN, —C(O)—N(R^{c})—, —S(O)—N(R^{c})₂, —S(O)—N(R^{c})₂, —O—R^{c}, —S—R^{c}, —O—C(O)—R^{c}, —C(O)—R^{c}, —C(O)—O—R^{c}, —S(O)—R^{c}, —S(O)₂—R^{c}, —C(O)—N(R^{c})₂, —N(R^{c})—C(O)—R^{c}, —N(R^{c})—S(O)—R^{c}, —N(R^{c})—S(O)²—R^{c}, and C₁₋₆ alkyl that is optionally substituted with one or more groups independently selected from oxo and halogen;
each R^{c} of formula (14) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —C(O)—R^{d}, —C(O)—R^{d}, —C(O)—O—R^{d}, —S(O)—R^{d}, —S(O)₂—R^{d}, —C(O)—N(R^{d})₂, —N(R^{d})—C(O)—R^{d}, —N(R^{d})—S(O)—R^{d}, —N(R^{d})—S(O)₂—R^{d}, and C₁₋₆ alkyl, and the carbocyclic ring and C₁₋₆ alkyl are optionally substituted with one or more groups independently selected from oxo, halogen, C₁₋₆ alkyl, cyano, —N(R^{d})₂, —O—R^{d}, a heterocycle, and a carbocyclic ring that is optionally substituted with one or more groups independently selected from halogen and C₁₋₆ alkyl;
each R^{d} in formula (14) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is independently optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{d}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{e} in formula (14) is independently selected from oxo, C₁₋₆ alkyl, C₁₋₆ alkenyl, C₁₋₆ alkynyl, a carbocyclic ring, a heterocycle, aryl, heteroaryl, fluorine, chlorine, bromine, iodine, —NO, —N(R^{f})₂, —CN, —C(O)—N(R^{f})₂, —S(O)—N(R^{f})₂, —S(O)₂—N(R^{f})₂, —O—R^{f}, —S—R^{f}, —O—C(O)—R^{f}, —O—C(O)—O—R^{f}, —C(O)—R^{f}, —C(O)—O—R^{f}, —S(O)—R^{f}, —S(O)₂—R^{f}, —O—C(O)—N(R^{f})₂, —N(R^{f})—C(O)—OR^{f}, —N(R^{f})—C(O)—N(R^{f})₂, —N(R^{f})—C(O)—R^{f}, —N(R^{f})—S(O)—R^{f}, —N(R^{f})—S(O)₂—R^{f}, —N(R^{f})—S(O)—N(R^{f})₂, and —N(R^{f})—S(O)₂—N(R^{f})₂, wherein any of C₁₋₆ alkyl, C₂₋₆ alkenyl, C₂₋₆ alkynyl, carbocyclic ring, heterocycle, aryl, and
heteroaryl is optionally substituted with one or more groups independently selected from oxo, halogen, —NO₂, —N(R^{f})₂, —CN, —C(O)—N(R^{f})₂, —S(O)—N(R^{f})₂, —S(O)₂—N(R^{f})₂, —O—R^{f}, —S—R^{f}, —O—C(O)—R^{f}, —C(O)—R^{f}, —C(O)—O—R^{f}, —S(O)—R^{f}, —S(O)₂—R^{f}, —C(O)—N(R^{f})₂, —N(R^{f})—C(O)—R^{f}, —N(R^{f})—S(O)—R^{f}, —N(R^{f})—S(O)₂—R^{f}, and a carbocyclic ring, and C₁₋₆ alkyl that is optionally substituted with one or more groups independently selected from oxo and halogen;
each R^{f} in formula (I) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{g})₂, —CN, —C(O)—N(R^{g})₂, —S(O)—N(R^{g})₂, —S(O)₂—N(R^{g})₂, —O—R^{g}, —S—R^{g}, —O—C(O)—R^{g}, —C(O)—R^{g}, —C(O)—O—R^{g}, —S(O)—R^{g}, —S(O)₂—R^{g}, —C(O)—N(R^{g})₂, —N(R^{g})—C(O)—R^{g}, —N(R^{g})—S(O)—R^{g}, —N(R^{g})—S(O)₂—R^{g}, and C₁₋₆ alkyl, and the carbocyclic ring and C₁₋₆ alkyl are optionally substituted with one or more groups independently selected from oxo, halogen, C₁₋₆ alkyl, cyano, —N(R^{g})₂, —O—R^{g}, a heterocycle, and a carbocyclic ring that is optionally substituted with one or more groups independently selected from halogen and C₁₋₆ alkyl;
each R¹ in formula (14) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{g}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{h} in formula (14) is independently selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkoxy, and C₁₋₃ alkyl that is optionally substituted with one or more groups independently selected from halogen;
R¹ in formula (15) is selected from C₆₋₂₀ aryl, C₁₋₂₀ heteroaryl, —(C₆₋₂₀ aryl)-(C₁₋₂₀ heteroaryl), and —(C₁₋₂₀ heteroaryl)-(C₁₋₂₀ heteroaryl), wherein each of C₆₋₂₀ aryl, C₁₋₂₀ heteroaryl, —(C₆₋₂₀ aryl)-(C₁₋₂₀ heteroaryl), and (C₁₋₂₀ heteroaryl)-(C₁₋₂₀ heteroaryl) is independently optionally substituted with one or more substituents independently selected from R^{c}, oxo, fluorine, chlorine, bromine, iodine, —NO₂, —N(R^{a})₂, —CN, —C(O)—N(R^{a})₂, —S(O)—N(R^{a})₂, —S(O)₂—N(R^{a})₂, —O—R^{a}, —S—R^{a}, —O—C(O)—R^{a}, —O—C(O)—O—R^{a}, —C(O)—R^{a}, —C(O)—O—R^{a}, —S(O)—R^{a}, —S(O)₂—R^{a}, —O—C(O)—N(R^{a})₂, —N(R^{a})—C(O)—OR^{a}, —N(R^{a})₂—C(O)—N(R^{a}), —N(R^{a})—C(O)—R^{a}, —N(R^{a})—S(O)—R^{a}, —N(R^{a})—S(O)—R^{a}, —N(R^{a})—S(O)—N(R^{a})₂, and —N(R^{a})—S(O)₂—N(R^{a} 2;
R² in formula (15) is C₁₋₁₂ alkyl, C₂₋₁₂ alkenyl, C₂₋₁₂ alkynyl, a 3- to 12-membered carbocyclic ring, or a 3- to 12-membered heterocycle, wherein each of C₁₋₁₂ alkyl, C₂₋₁₂ alkenyl, C₂₋₁₂ alkynyl, 3- to 12-membered carbocyclic ring, and 3- to 12-membered heterocycle of R² is optionally substituted with one or more R^{b};
R³ in formula (15) is C₁₋₄ alkyl, C₂₋₄ alkenyl, C₂₋₄ alkynyl, a 3- to 5-membered carbocyclic ring, a 3- to 5-membered heterocycle, —C(O)—N(R^{e})₂, —S(O)—N(R^{e})₂, —S(O)₂—N(R^{e})₂, —C(O)—R^{e}, —C(O)—OR^{e}, —S(O)—R^{e}, or —S(O)₂—R^{e}, wherein any of C₁₋₄ alkyl, C₂₋₄ alkenyl, C₂₋₄ alkynyl, 3- to 5-membered carbocyclic ring, and 3- to 5-membered heterocycle is optionally substituted with one or more substituents independently selected from fluorine, chlorine, bromine, iodine, a 3- to 5-membered carbocyclic ring, —C(O)—N(R^{e})₂, —S(O)—N(R^{e})₂, —S(O)₂—N(R^{e})₂, —O—R^{e}, —S—R^{e}, —O—C(O)—R, —O—C(O)—O—R^{e}, —C(O)—R^{e}, —C(O)—O—R^{e}, —S(O)—R^{e}, —S(O)₂—R^{e}, —O—C(O)—N(R^{e})₂, —N(R^{e})—C(O)—OR^{e}, —N(R^{e})—C(O)—N(R^{e})₂, —N(R^{e})—C(O)—R^{e}, —N(R^{e})—S(O)—R^{e}, —N(R^{e})—S(O)₂—R^{e}, —N(R^{e})—S(O)—N(R^{e})₂, and —N(R^{e})—S(O)₂—N(R^{e})₂;
each R^{a} in formula (15) is independently selected from a hydrogen atom, C₁₋₆ alkyl, C₂₋₆ alkenyl, C₂₋₆ alkynyl, a carbocyclic ring, and a heterocycle, wherein each of C₁₋₆ alkyl, C₂₋₆ alkenyl, C₂₋₆ alkynyl, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{a}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{b} in formula (15) is independently selected from oxo, C₁₋₆ alkyl, C₂₋₆ alkenyl, C₂₋₆ alkynyl, a carbocyclic ring, a heterocycle, aryl, heteroaryl, fluorine, chlorine, bromine, iodine, —NO₂, —N(R^{c})₂, —CN, —C(O)—N(R^{c})₂, —S(O)—N(R^{c})₂, —S(O)²—N(R^{c})₂, —O—R^{c}, —S—R^{c}, —O—C(O)—R^{c}, —O—C(O)—O—R^{c}, —C(O)—R^{c}, —C(O)—O—R^{c}, —S(O)—R^{c}, —S(O)₂—R^{c}, —O—C(O)—N(R^{c})₂, —N(R^{c})—C(O)—OR^{c}, —N(R^{c})—C(O)—N(R^{c})₂, —N(R^{c})—C(O)—R^{c}, —N(R^{c})—S(O)—R^{c}, —N(R^{c})—S(O)₂—R^{c}, —N(R^{c})—S(O)—N(R^{c})₂, and —N(R^{c})—S(O)₂—N(R^{c})₂, wherein any of C₁₋₆ alkyl, C₂₋₆ alkenyl, C₂₋₆ alkynyl, carbocyclic ring, heterocycle, aryl, and
heteroaryl is optionally substituted with one or more groups independently selected from oxo, halogen, —NO₂, —N(R^{c})₂, —CN, —C(O)—N(R^{c})₂, —S(O)—N(R^{c})₂, —S(O)₂—N(R^{c})₂, —O—R^{c}, —S—R^{c}, —O—C(O)—R^{c}, —C(O)—R^{c}, —C(O)—O—R^{c}, —S(O)—R^{c}, —S(O)₂—R^{c}, —C(O)—N(R^{c})₂, —N(R^{c})—C(O)—R^{c}, —N(R^{c})—S(O)—R^{c}, —N(R^{c})—S(O)₂—R^{c}, and C₁₋₆ alkyl that is optionally substituted with one or more groups independently selected from oxo and halogen;
each R^{c} in formula (15) is independently selected from a hydrogen atom, C₁₋₆ alkyl, C₂₋₆ alkenyl, C₂₋₆ alkynyl, a carbocyclic ring, and a heterocycle, wherein any of C₁₋₆ alkyl, C₂₋₆ alkenyl, C₂₋₆ alkynyl, carbocyclic ring, and heterocycle is optionally substituted with one or more groups independently selected from oxo, a carbocyclic ring, a heterocycle, halogen, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —O—C(O)—R^{d}, —C(O)—R^{d}, —C(O)—O—R^{d}, —S(O)—R^{d}, —S(O)₂—R^{d}, —C(O)—N(R^{d})₂, —N(R^{d})—C(O)—R^{d}, —N(R^{d})—S(O)—R^{d}, —N(R^{d})—S(O)₂—R^{d}, and C₁₋₆ alkyl, and the carbocyclic ring and C₁₋₆ alkyl are optionally substituted with one or more groups independently selected from oxo, halogen, C₁₋₆ alkyl, cyano, —N(R^{d})₂, —O—R^{d}, a heterocycle, and a carbocyclic ring that is optionally substituted with one or more groups independently selected from halogen and C₁₋₆ alkyl;
each of R^{d} in formula (15) is independently selected from a hydrogen atom, C₁₋₆ alkyl, C₂₋₆ alkenyl, C₂₋₆ alkynyl, C₁₋₆ alkoxy, a carbocyclic ring, and a heterocycle, wherein each of C₁₋₆ alkyl, C₂₋₆ alkenyl, C₂₋₆ alkynyl, C₁₋₆ alkoxy, carbocyclic ring, and heterocycle is optionally substituted with one or more groups independently selected from oxo, halogen, amino, a hydroxyl group, C₁₋₆ alkoxy, a carbocyclic ring, a heterocycle, and C₁₋₆ alkyl that is optionally substituted with one or more groups independently selected from oxo and halogen; or
two R^{d}, together with the nitrogen to which they are attached, form a heterocycle optionally substituted with one or more groups independently selected from oxo, halogen, and C₁₋₃ alkyl that is optionally substituted with one or more groups independently selected from oxo and halogen;
each R^{e} in formula (15) is selectively selected from a hydrogen atom, C₁₋₄ alkyl, C₂₋₄ alkenyl, C₂₋₄ alkynyl, and C₂₋₅ cycloalkyl, wherein each of C₁₋₄ alkyl, C₂₋₄ alkenyl, C₂₋₄ alkynyl, and C₂₋₅ cycloalkyl is optionally substituted with one or more groups independently selected from oxo, halogen, amino, a hydroxyl group, C₁₋₃ alkoxy, and C₁₋₃ alkyl that is optionally substituted with one or more groups independently selected from halogen,
provided that if R² is carboxymethyl or 2-carboxyethyl, R¹ is not unsubstituted phenyl,
or a prodrug thereof or a pharmaceutically acceptable salt thereof.

55. The pharmaceutical composition of claim 31, wherein the low molecular weight compound is a compound represented by the following (Table 12)
TABLE 12-1 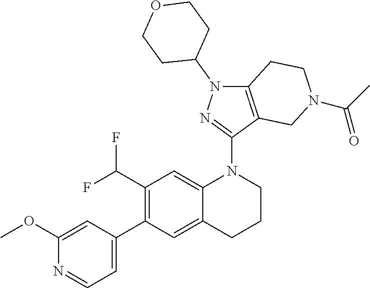 [SMILES: COc1cc(-c2cc3c(cc2C(F)F)N(c2nn(C4CCOCC4)c4c2CN(C(C)=O)CC4)CCC3)ccn1] 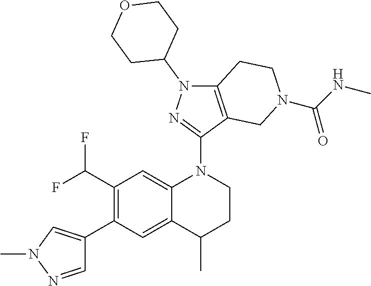 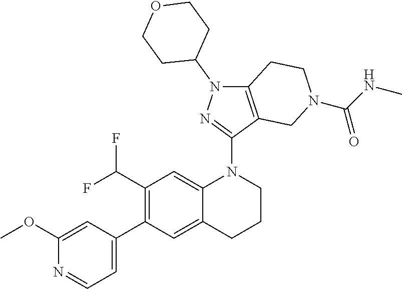 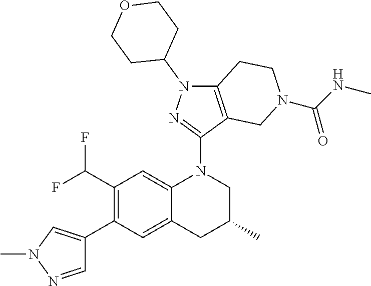 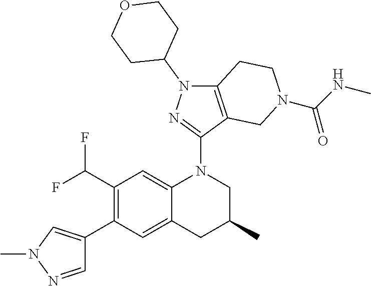 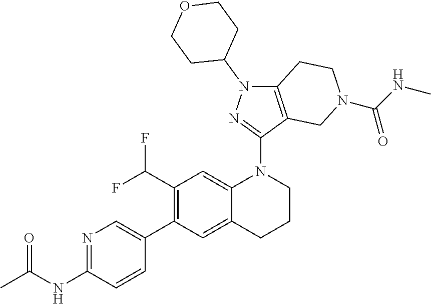 [SMILES: CNC(=O)N1CCc2c(c(N3CCCc4cc(-c5ccc(NC(C)=O)nc5)c(C(F)F)cc43)nn2C2CCOCC2)C1] 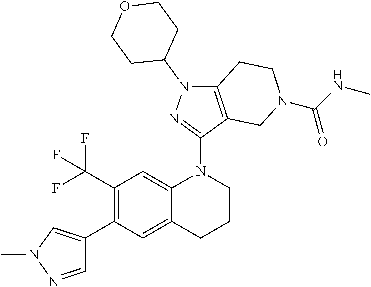 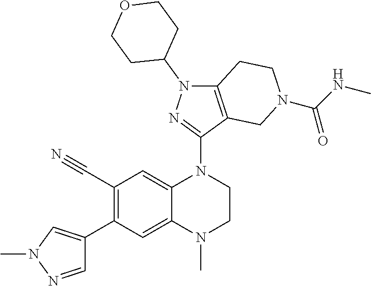 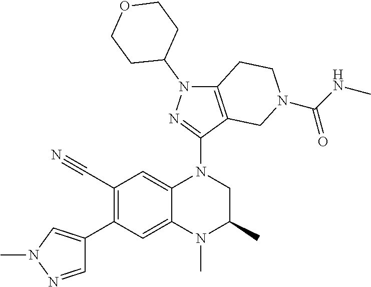 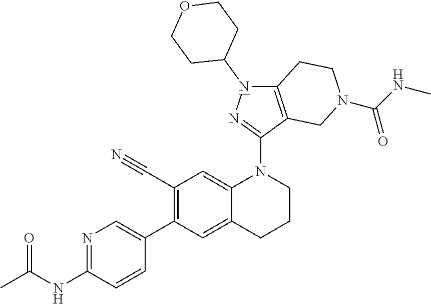 [SMILES: CNC(=O)N1CCc2c(c(N3CCCc4cc(-c5ccc(NC(C)=O)nc5)c(C#N)cc43)nn2C2CCOCC2)C1] 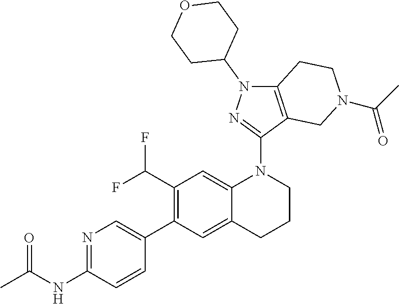 [SMILES: CC(=O)Nc1ccc(-c2cc3c(cc2C(F)F)N(c2nn(C4CCOCC4)c4c2CN(C(C)=O)CC4)CCC3)cn1] 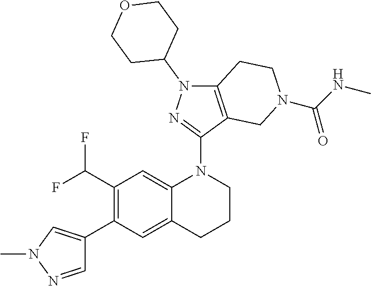 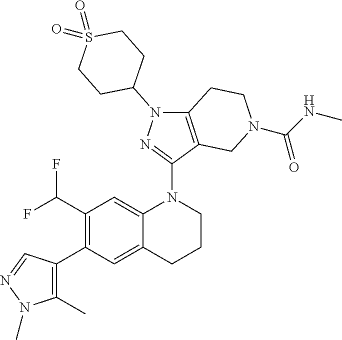 [SMILES: CNC(=O)N1CCc2c(c(N3CCCc4cc(-c5cnn(C)c5C)c(C(F)F)cc43)nn2C2CCS(=O)(=O)CC2)C1] 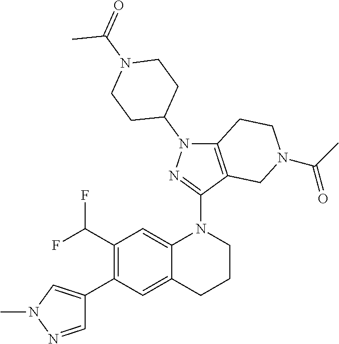 [SMILES: CC(=O)N1CCC(n2nc(N3CCCc4cc(-c5cnn(C)c5)c(C(F)F)cc43)c3c2CCN(C(C)=O)C3)CC1] 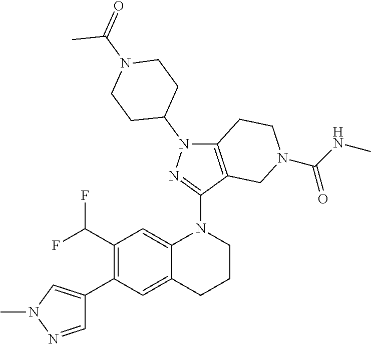
TABLE 12-2 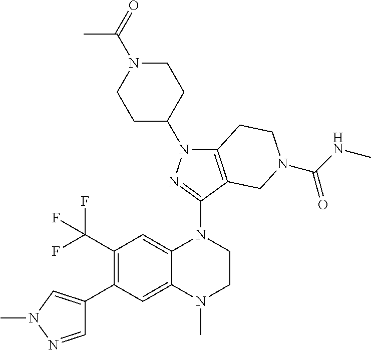 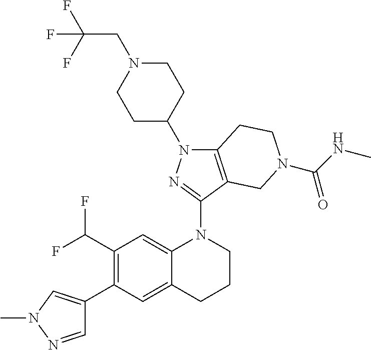 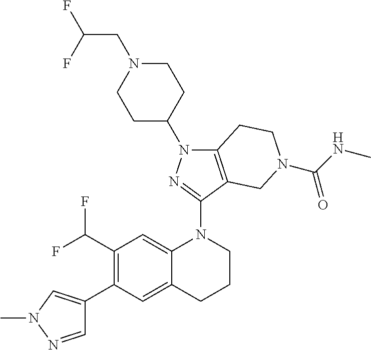 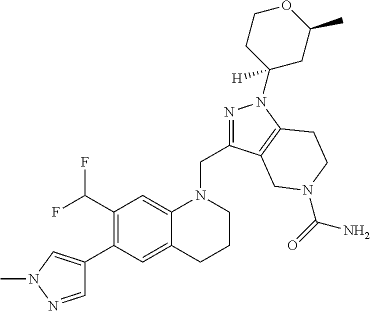 [SMILES: C[C@H]1C[C@@H](n2nc(CN3CCCc4cc(-c5cnn(C)c5)c(C(F)F)cc43)c3c2CCN(C(N)=O)C3)CCO1] 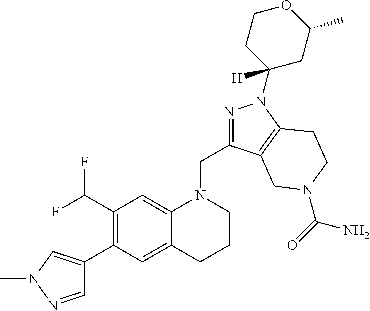 [SMILES: C[C@@H]1C[C@H](n2nc(CN3CCCc4cc(-c5cnn(C)c5)c(C(F)F)cc43)c3c2CCN(C(N)=O)C3)CCO1] 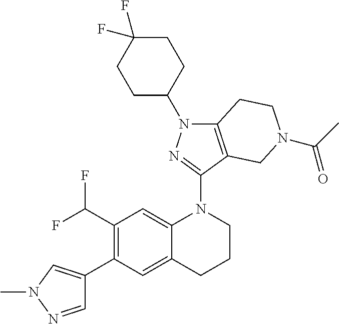 [SMILES: CC(=O)N1CCc2c(c(N3CCCc4cc(-c5cnn(C)c5)c(C(F)F)cc43)nn2C2CCC(F)(F)CC2)C1] 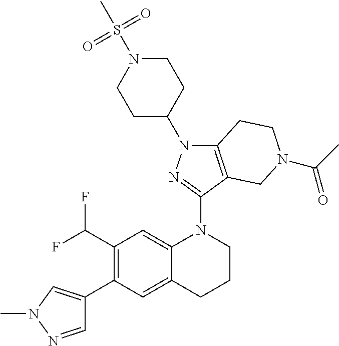 [SMILES: CC(=O)N1CCc2c(c(N3CCCc4cc(-c5cnn(C)c5)c(C(F)F)cc43)nn2C2CCN(S(C)(=O)=O)CC2)C1] 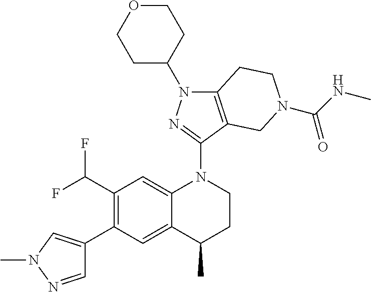 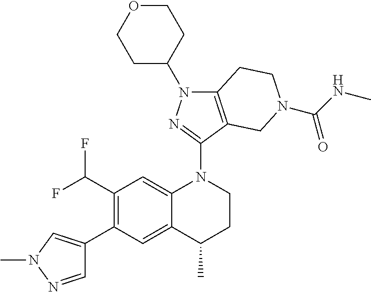 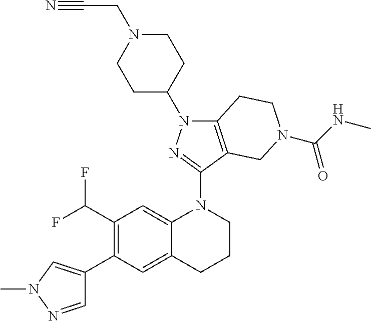 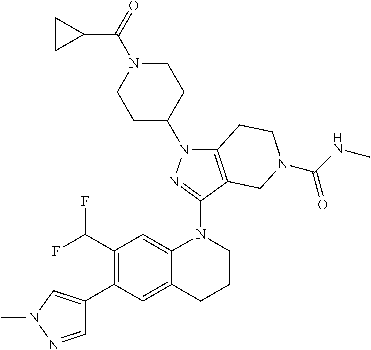 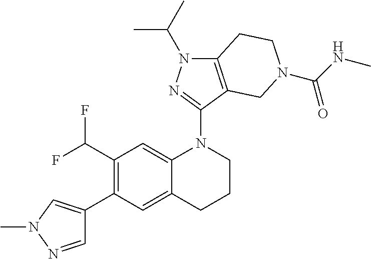 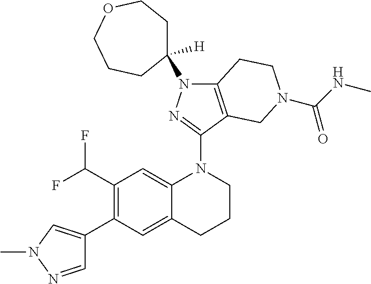 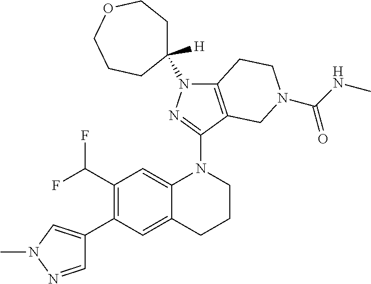 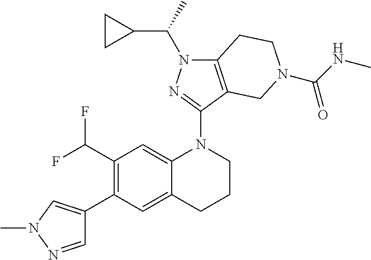
TABLE 12-3 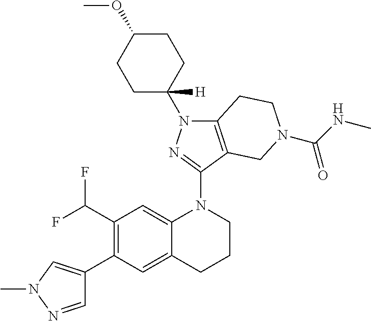 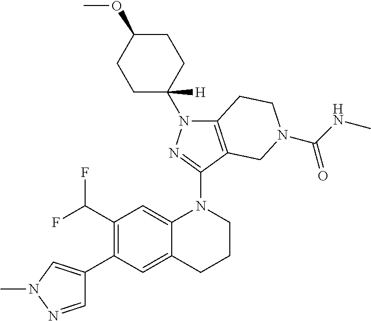 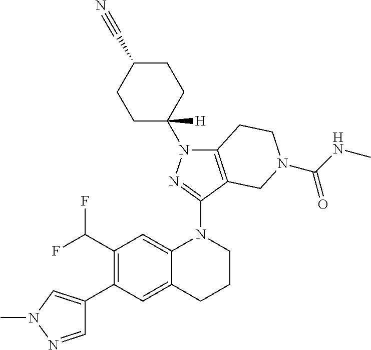 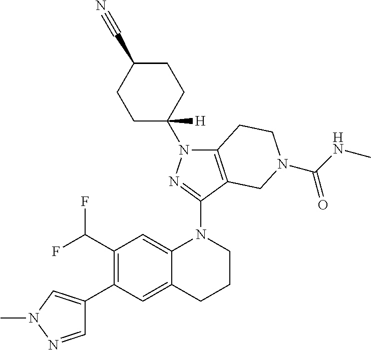 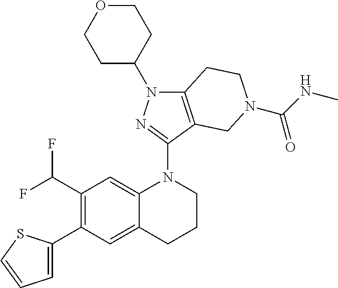 [SMILES: CNC(=O)N1CCc2c(c(N3CCCc4cc(-c5cccs5)c(C(F)F)cc43)nn2C2CCOCC2)C1] 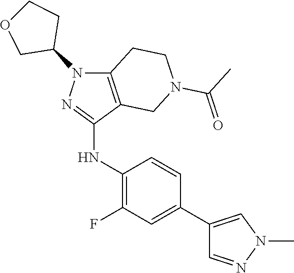 [SMILES: CC(=O)N1CCc2c(c(Nc3ccc(-c4cnn(C)c4)cc3F)nn2[C@@H]2CCOC2)C1]
or a pharmaceutically acceptable salt thereof.

56. The pharmaceutical composition of claim 31, wherein the low molecular weight compound is a compound represented by formula (14) 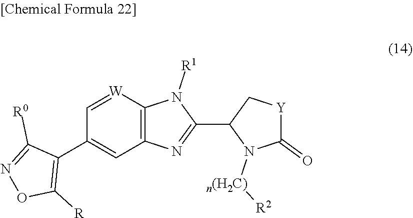
wherein
R⁰ and R are the same or different, each a hydrogen atom, or C₁₋₆ alkyl, which is unsubstituted or substituted with OH, —OC(O)R′, or OR′ (wherein R′ is unsubstituted C₁₋₆ alkyl);
W is N or CH;
R¹ is an unsubstituted or substituted group, which is C-linked 4- to 6-membered heterocyclyl, C₃₋₆ cycloalkyl, or C₁₋₆ alkyl that is unsubstituted or substituted with C₆₋₁₀ aryl, 5- to 12-membered N-containing heteroaryl, C₃₋₆ cycloalkyl, OH, —OC(O)R′, or OR′ (wherein R′ is as defined above, or a group represented by: 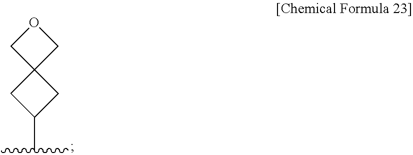 [SMILES: *C1CC2(COC2)C1]
Y is —CH₂—, —CH₂CH₂—, or CH₂CH₂CH₂—;
n is 0 or 1; and
R² is a group selected from C₆₋₁₀ aryl, 5- to 12-membered N-containing heteroaryl, C₃₋₆ cycloalkyl, and C₅₋₆ cycloalkenyl, which are unsubstituted or substituted, and the C₆₋₁₀ aryl may be fused to a 5- or 6-membered heterocycle,
or a prodrug thereof or a pharmaceutically acceptable salt thereof.

57. The pharmaceutical composition of claim 31, wherein the low molecular weight compound is a compound represented by 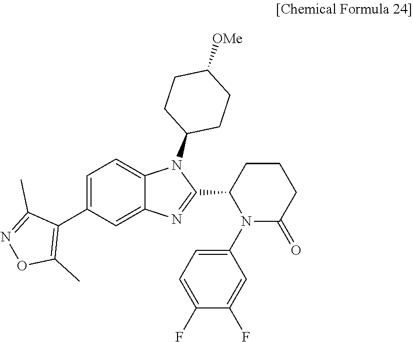 [SMILES: CO[C@H]1CC[C@H](n2c([C@@H]3CCCC(=O)N3c3ccc(F)c(F)c3)nc3cc(-c4c(C)noc4C)ccc32)CC1]
or a pharmaceutically acceptable salt thereof.

58. The pharmaceutical composition of claim 31, wherein the compound is a compound represented by formula (15) 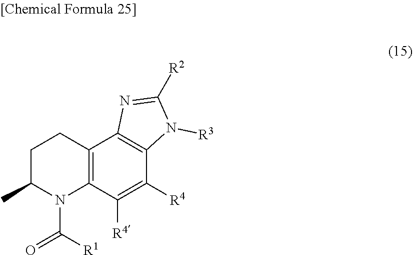
wherein
R¹ is —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, or OR⁵;
R² is hydrogen, —C₁₋₆ alkyl, —C₂₋₆ alkenyl, C₂₋₆ alkynyl, —C₃₋₈ cycloalkyl, —C₄₋₈ cycloalkenyl, heterocyclyl, heteroaryl, or aryl, wherein each of alkyl, alkenyl, alkynyl, cycloalkyl, cycloalkenyl, heterocyclyl, heteroaryl, and aryl is optionally substituted with one or more R⁶, and a —C₁₋₆ alkyl group has one or more methylene units optionally substituted with —NR⁶—, —O—, or —S—;
R³ is hydrogen, —C₁₋₆ alkyl, —C₂₋₆ alkenyl, —C₂₋₆ alkynyl, —C₃₋₈ cycloalkyl, —C₄₋₈ cycloalkenyl, spirocycloalkyl, spiroheterocyclyl, heterocyclyl, heteroaryl, or aryl, wherein each of alkyl, alkenyl, alkynyl, cycloalkyl, cycloalkenyl, spirocycloalkyl, spiroheterocyclyl, heterocyclyl, heteroaryl, and aryl is optionally substituted with one or more R⁷;
R⁴ and R^{4′} are each independently —H, halogen, —OH, —CN, or NH₂;
R⁵ is —C₁₋₆ alkyl, —C₃₋₈ cycloalkyl, heterocyclyl, aryl, or heteroaryl;
R⁶ and R⁷ are each independently, for each instance, hydrogen, —C₁₋₆ alkyl, —C₃₋₈ cycloalkyl, —C₄₋₈ cycloalkenyl, heterocyclyl, aryl, spirocycloalkyl, spiroheterocyclyl, heteroaryl, —OH, halogen, oxo, —CN, —SR⁸, —OR⁸, —(CH₂)ₙ—OR⁸, —NHR⁸, —NR⁸R⁹, —S(O)₂NR⁸R⁹, —S(O)₂R^{8′}, —C(O)R^{8′}, —C(O)OR⁸, —C(O)NR⁸R⁹, —NR⁸C(O)R^{9′}, —NR⁸S(O)₂R^{9′}, —S(O)R^{8′}, —S(O)NR⁸R⁹, or NR⁸S(O)R^{9′}, wherein each of alkyl, cycloalkyl, heterocyclyl, spirocycloalkyl, spiroheterocyclyl, heteroaryl, and aryl is optionally substituted with one or more R¹⁰;
wherein any two R⁶ or any two R⁷, when on non-adjacent atoms, can attach and form bridged cycloalkyl or heterocyclyl, wherein any two R⁶ or any two R⁷, when on adjacent atoms, can attach and form cycloalkyl, heterocyclyl, aryl, or heteroaryl;
R⁸ and R⁹ are each independently, for each instance, —H, —C₁₋₆ alkyl, —C₂₋₆ alkenyl, —C₂₋₆ alkynyl, —C₃₋₈ cycloalkyl, —C₄₋₈ cycloalkenyl, heterocyclyl, aryl, or heteroaryl, wherein each of alkyl, alkenyl, alkynyl, cycloalkyl, cycloalkenyl, heterocyclyl, aryl, and heteroaryl is optionally substituted with one or more R¹⁰ or R¹¹; or
R⁸ and R⁹, attached to an atom to which they are both attached, may form —C₃₋₈ cycloalkyl, —C₄₋₈ cycloalkenyl, spirocycloalkyl, spiroheterocyclyl, heterocyclyl, heteroaryl, or aryl, and the formed —C₃₋₈ cycloalkyl, —C₄₋₈ cycloalkenyl, spirocycloalkyl, spiroheterocyclyl, heterocyclyl, heteroaryl, or aryl is optionally substituted with one or more R¹⁰ or R¹¹;
R^{8′} and R^{9′} are each independently, for each instance, —C₁₋₆ alkyl, —C₂₋₆ alkenyl, —C₂₋₆ alkynyl, —C₃₋₈ cycloalkyl, —C₄₋₈ cycloalkenyl, heterocyclyl, aryl, or heteroaryl, wherein each of alkyl, alkenyl, alkynyl, cycloalkyl, cycloalkenyl, heterocyclyl, aryl, and heteroaryl is optionally substituted with one or more R¹⁰ or R¹¹; or
R⁸ and R^{9′}, attached to an atom to which they are both attached, may form —C₃₋₈ cycloalkyl, —C₄₋₈ cycloalkenyl, spirocycloalkyl, spiroheterocyclyl, heterocyclyl, heteroaryl, or aryl, and —C₃₋₈ cycloalkyl, —C₄₋₈ cycloalkenyl, spirocycloalkyl, spiroheterocyclyl, heterocyclyl, heteroaryl, or aryl is optionally substituted with one or more R¹⁰ or R¹¹;
R¹⁰ and R¹¹ are each independently, for each instance, hydrogen, —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, —OH, halogen, oxo, —NO₂, —CN, —NH₂, —OC₁₋₆ alkyl, —NHC₁₋₆ alkyl, —N(C₁₋₆ alkyl)₂, —S(O)₂NH(C₁₋₆ alkyl), —S(O)₂N(C₁₋₆ alkyl)₂, —S(O) 2 C₁₋₆ alkyl, —C(O) C₁₋₆ alkyl, —C(O)NH₂, —C(O)NH(C₁₋₆ alkyl), —C(O)N(C₁₋₆ alkyl)₂, —C(O) OC₁₋₆ alkyl, —N(C₁₋₆ alkyl) SO₂C₁₋₆ alkyl, —S(O)(C₁₋₆ alkyl), —S(O)N(C₁₋₆ alkyl)₂, or N(C₁₋₆ alkyl)S(O)(C₁₋₆ alkyl), wherein each of alkyl, alkenyl, alkynyl, cycloalkyl, cycloalkenyl, heterocyclyl, heteroaryl, and aryl is optionally substituted with one or more R¹²;
wherein any two R¹⁰ or any two R¹¹, when on non-adjacent atoms, can attach and form bridged cycloalkyl or heterocyclyl;
wherein any two R¹⁰ or any two R¹¹, when on adjacent atoms, can attach and form cycloalkyl, heterocyclyl, aryl, or heteroaryl;
R¹² is each independently, for each instance, —H, —C₁₋₆ alkyl, —C₂₋₆ alkenyl, —C₂₋₆ alkynyl, —C₃₋₈ cycloalkyl, —C₄₋₈ cycloalkenyl, heterocyclyl, heteroaryl, aryl, —OH, halogen, oxo, —NO₂, —CN, —NH₂, —OC₁₋₆ alkyl, —NHC₁₋₆ alkyl, —N(C₁₋₆ alkyl)₂, —S(O)₂NH(C₁₋₆ alkyl), —S(O)₂N(C₁₋₆ alkyl)₂, —S(O)₂C₁₋₆ alkyl, —C(O) C₁₋₆ alkyl, —C(O)NH₂, —C(O)NH(C₁₋₆ alkyl), —C(O)N(C₁₋₆ alkyl), —C(O) OC₁₋₆ alkyl, —N(C₁₋₆ alkyl)SO₂C₁₋₆ alkyl, —S(O)(C₁₋₆ alkyl), —S(O)N(C₁₋₆ alkyl)₂, or N(C₁₋₆ alkyl)S(O)(C₁₋₆ alkyl), and
n is an integer from 1 to 4,
or a prodrug thereof or a pharmaceutically acceptable salt thereof.

59. The pharmaceutical composition of claim 31, wherein the low molecular weight compound is a compound represented by the following (Table 13)
TABLE 13 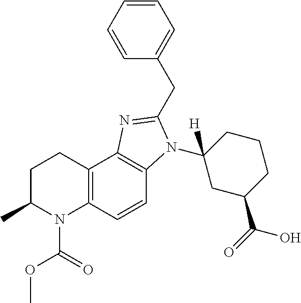 [SMILES: COC(=O)N1c2ccc3c(nc(Cc4ccccc4)n3[C@@H]3CCC[C@@H](C(=O)O)C3)c2CC[C@@H]1C] 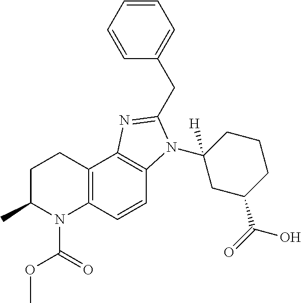 [SMILES: COC(=O)N1c2ccc3c(nc(Cc4ccccc4)n3[C@H]3CCC[C@H](C(=O)O)C3)c2CC[C@@H]1C] 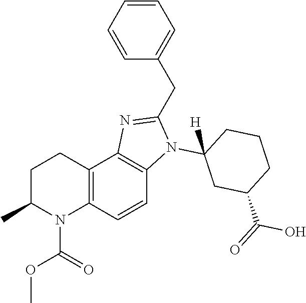 [SMILES: COC(=O)N1c2ccc3c(nc(Cc4ccccc4)n3[C@@H]3CCC[C@H](C(=O)O)C3)c2CC[C@@H]1C] 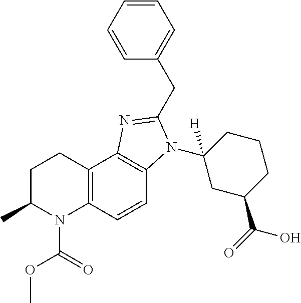 [SMILES: COC(=O)N1c2ccc3c(nc(Cc4ccccc4)n3[C@H]3CCC[C@@H](C(=O)O)C3)c2CC[C@@H]1C] 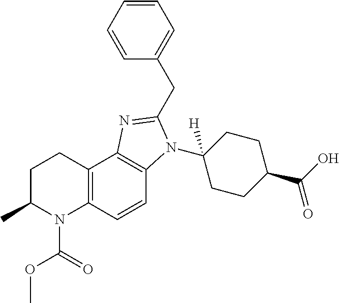 [SMILES: COC(=O)N1c2ccc3c(nc(Cc4ccccc4)n3[C@H]3CC[C@@H](C(=O)O)CC3)c2CC[C@@H]1C] 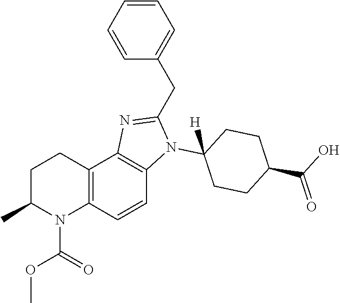 [SMILES: COC(=O)N1c2ccc3c(nc(Cc4ccccc4)n3[C@H]3CC[C@H](C(=O)O)CC3)c2CC[C@@H]1C] 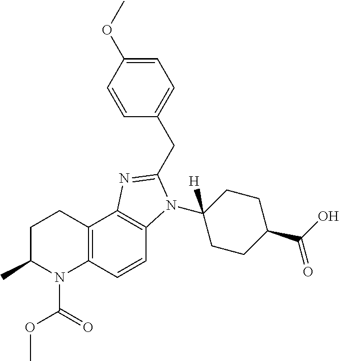 [SMILES: COC(=O)N1c2ccc3c(nc(Cc4ccc(OC)cc4)n3[C@H]3CC[C@H](C(=O)O)CC3)c2CC[C@@H]1C] 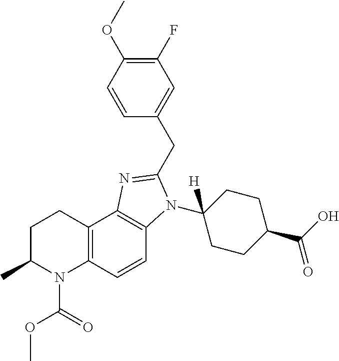 [SMILES: COC(=O)N1c2ccc3c(nc(Cc4ccc(OC)c(F)c4)n3[C@H]3CC[C@H](C(=O)O)CC3)c2CC[C@@H]1C] 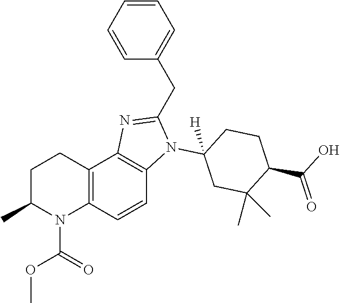 [SMILES: COC(=O)N1c2ccc3c(nc(Cc4ccccc4)n3[C@H]3CC[C@@H](C(=O)O)C(C)(C)C3)c2CC[C@@H]1C] 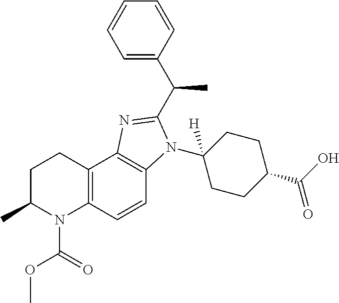 [SMILES: COC(=O)N1c2ccc3c(nc([C@H](C)c4ccccc4)n3[C@H]3CC[C@H](C(=O)O)CC3)c2CC[C@@H]1C] 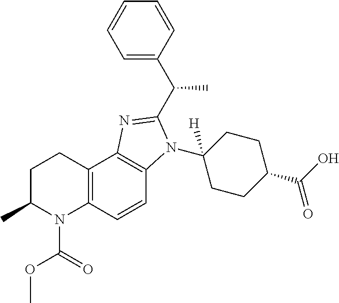 [SMILES: COC(=O)N1c2ccc3c(nc([C@@H](C)c4ccccc4)n3[C@H]3CC[C@H](C(=O)O)CC3)c2CC[C@@H]1C] 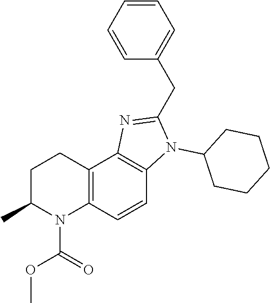 [SMILES: COC(=O)N1c2ccc3c(nc(Cc4ccccc4)n3C3CCCCC3)c2CC[C@@H]1C]
or a pharmaceutically acceptable salt thereof.

60. The pharmaceutical composition of claim 31, wherein the low molecular weight compound is a compound represented by formula (16) 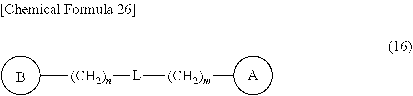
wherein
ring B is a group having the following structure; 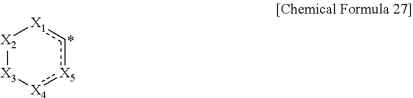
one of ring atoms X₂ and X₃ is N(R^{X1}), and the other one of the ring atoms X₂ and X₃ is C(═O);
ring atom X₁ is selected from N(R^{X1}), C(R^{X2}), and C(═O), and ring atoms X₄ and X₅ are each independently selected from N(R^{X1}), C(R^{X3}), and C(═O); wherein at least one of the ring atoms X₁, X₄, and X₅ is different from N(R^{X1}) and C(═O); and wherein if X₃ and X₅ are C(═O), X₄ is N(R^{X1}), and X₁ is C(R^{X2}), X₂ is N(H);
each [symbol]
[Chemical Formula 28]
is independently a single bond or a double bond; wherein at least one of any two adjacent bonds [symbol]
[Chemical Formula 29]
is a single bond;
each R^{X1} is independently selected from hydrogen, C₁₋₅ alkyl, —CO(C₁₋₅ alkyl), —(C₀₋₃ alkylene)-aryl, and heteroaryl,
wherein aryl in the —(C₀₋₃ alkylene)-aryl and the heteroaryl are each optionally substituted with one or more groups R^{X11};
R^{X2} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O— (C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
two groups R^{X3} are linked to each other and, together with the ring carbon to which they are attached, form a 5- or 6-membered cyclyl group optionally substituted with one or more groups R^{X31}, or two groups R^{X3} are each independently selected from hydrogen, C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl) (C₁₋₅ alkyl), halogen, C₁₋₅ haloalkyl, —O—(C₁₋₅ haloalkyl), —CF₃, —CN, —NO₂, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl) (C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl) (C₁₋₅ alkyl), —NH—SO—(C₁₋₅ alkyl), and —N(C₁₋₅ alkyl)-SO₂—(C₁₋₅ alkyl);
each R^{X11} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O— (C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl)-(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
each R^{X31} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O— (C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—N(C₁₋₅ alkyl), —(C₀₋₃ alkylene)-SO—N(C₁₋₅ alkyl) (C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
if ring B is attached to the remaining portion of the compound of formula (16) via a ring carbon atom marked with an asterisk (*), or if X₄ and X₅ are each C(R^{X3}) and two groups R^{X3} are linked to each other and, together with the ring carbon atom to which they are attach, form a 5- or 6-membered cyclyl group optionally substituted with one or more groups R^{X31}, ring B may be attached to the remaining portion of the compound of formula (16) via any carbocyclic ring atom of the 5- or 6-membered cyclyl group;
ring A is aryl or heteroaryl, wherein the aryl and the heteroaryl are optionally substituted with one or more groups R^{A}, wherein the heteroaryl is selected from 1,4-benzodioxanyl, benzoxanyl, 1,3-benzodioxolanyl, benzoxolanyl, and 1,5-benzodioxepanyl;
each R^{A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl) (C₁₋₅ alkyl), —(C₀₋₃ alkylene)-halogen, —(C₀₋₃ alkylene)-(C₁₋₅ haloalkyl), —(C₀₋₃ alkylene)-O— (C₁₋₅ haloalkyl), —(C₀₋₃ alkylene)-CF₃, —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl) (C₁₋₅ alkyl), —(C₀₋₃ alkylene)-NH—CO(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl) (C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-cycloalkyl, —(C₀₋₃ alkylene)-O-cycloalkyl, —(C₀₋₃ alkylene)-O(C₁₋₅ alkylene)-cycloalkyl, —(C₀₋₃ alkylene)-heterocycloalkyl, —(C₀₋₃ alkylene)-O-heterocycloalkyl, and —(C₀₋₃ alkylene)-O(C₁₋₅ alkylene)-heterocycloalkyl;
L is selected from —CO—N(R^{L1})—, —N(R^{L1})—CO—, —CO—O—, —O—CO—, —C(═N—R^{L2})—N(R^{L1})—, —N(R^{L1})—C(═N—R^{L2})—, —C(═S)—N(R^{L1})—, —N(R^{L1})—C(═S)—, —N(R^{L1})—CO—N(R^{L1})—, —O—CO—N(R^{L1})—, —N(R^{L1})—CO—O—, —N(R^{L1})—C(═N—R^{L2})—N(R^{L1}), —O—C(═N—R^{L2})—N(R^{L1})—, —N(R^{L1})—C(═N—R^{L2})—O—, —S—C(═N—R^{L2})—N(R^{L1})—, —N(R^{L1})—C(═N—R^{L2})—S—, —N(R^{L1})—C(═S)—N(R^{L1}), —O—C(═S)—N(R^{L1}), —N(R^{L1})—C(═S)—O—, —S—CO—N(R^{L1})—, and —N(R^{L1})—CO—S—;
each R^{L1} is independently selected from hydrogen and C₁₋₅ alkyl;
each R^{L2} is independently selected from hydrogen, C₁₋₅ alkyl, —CN, and —NO₂;
n is 0 or 1; and
m is 0 or 1,
or a prodrug thereof or a pharmaceutically acceptable salt thereof.

61. The pharmaceutical composition of claim 31, wherein the low molecular weight compound is a compound represented by the following (Table 14)
TABLE 14 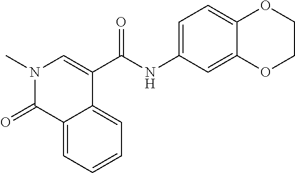 [SMILES: Cn1cc(C(=O)Nc2ccc3c(c2)OCCO3)c2ccccc2c1=O] 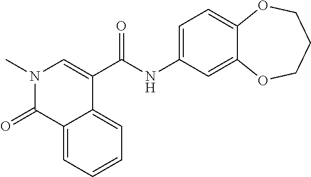 [SMILES: Cn1cc(C(=O)Nc2ccc3c(c2)OCCCO3)c2ccccc2c1=O] 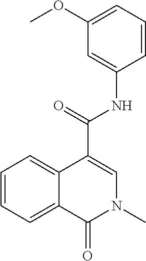 [SMILES: COc1cccc(NC(=O)c2cn(C)c(=O)c3ccccc23)c1] 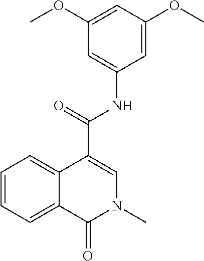 [SMILES: COc1cc(NC(=O)c2cn(C)c(=O)c3ccccc23)cc(OC)c1] 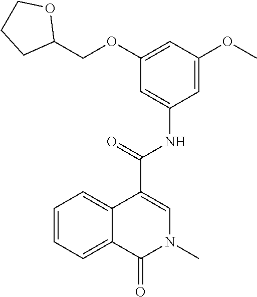 [SMILES: COc1cc(NC(=O)c2cn(C)c(=O)c3ccccc23)cc(OCC2CCCO2)c1] 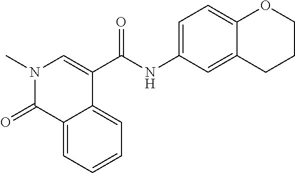 [SMILES: Cn1cc(C(=O)Nc2ccc3c(c2)CCCO3)c2ccccc2c1=O] 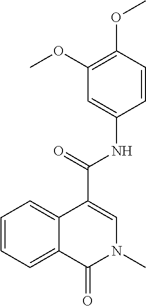 [SMILES: COc1ccc(NC(=O)c2cn(C)c(=O)c3ccccc23)cc1OC]
or a pharmaceutically acceptable salt thereof.

62. The pharmaceutical composition of claim 31, wherein the low molecular weight compound is a compound represented by formula (17) 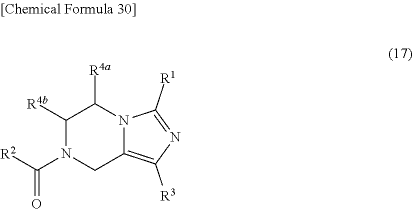
wherein
R¹ is hydrogen, or selected from alkyl, amino, alkoxy, heteroalkyl, cycloalkyl, heterocycloalkyl, halogen, haloalkyl, sulfonylalkyl, aryl, and heteroaryl, which is optionally substituted with 1, 2, or 3 group R⁵;
R² is hydrogen, or selected from alkyl, haloalkyl, amino, alkoxy, cycloalkyl, and heterocycloalkyl, which is optionally substituted with 1 or 2 group R⁶;
R³ is selected from alkyl, amino, alkoxy, heteroalkyl, cycloalkyl, heterocycloalkyl, carbonyl, sulfonyl, aryl, and heteroaryl, wherein the R³ is:
(a) optionally substituted with 1, 2, or 3 group R⁷, and
(b) optionally substituted with one R⁸;
R⁴³ and R^{4b} are hydrogen;
R⁵, R⁶, and R⁷ are each independently selected from alkyl, alkoxy, cyano, carboxy, halogen, haloalkyl, haloalkoxyl, hydroxy, and oxo;
R⁸ is selected from aryl, heteroaryl, and heterocycloalkyl, wherein the R⁸ is optionally substituted with 1, 2, or 3 group R¹⁰;
R¹⁰ is each independently selected from alkyl, cycloalkyl, (cycloalkyl)alkyl, heterocycloalkyl, (heterocycloalkyl)alkyl, aryl, (aryl)alkyl, (heteroaryl)alkyl, alkoxy, cyano, carboxy, halogen, haloalkyl, haloalkoxy, hydroxy, hydroxyalkyl, oxo, CONH₂, CONHCH₃, SO₂CH₃, and SO₂NH₂,
or a prodrug thereof or a pharmaceutically acceptable salt thereof.

63. The pharmaceutical composition of claim 31, wherein the low molecular weight compound is a compound represented by the following (Table 15)
TABLE 15 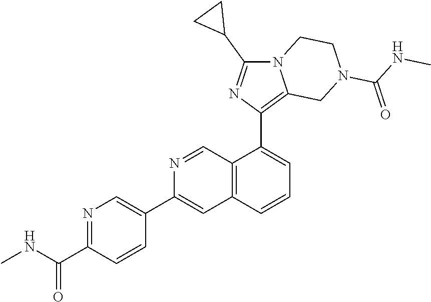 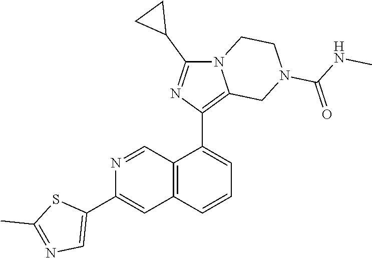 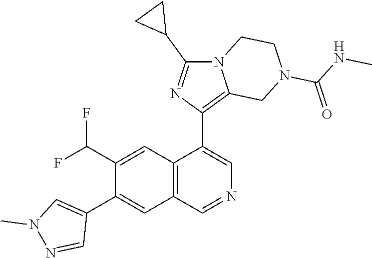 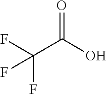 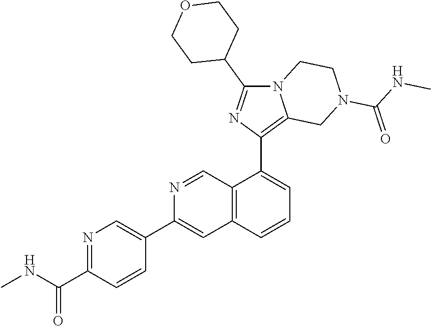 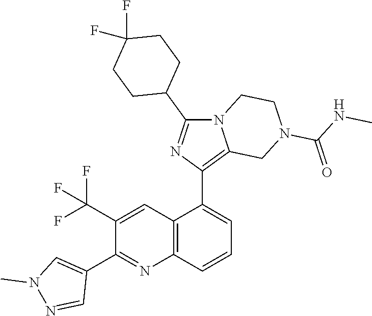 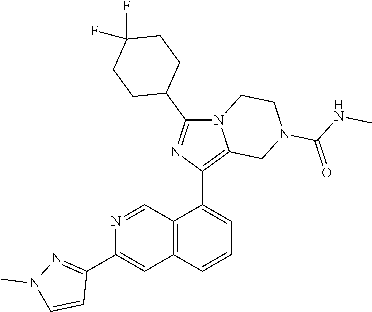 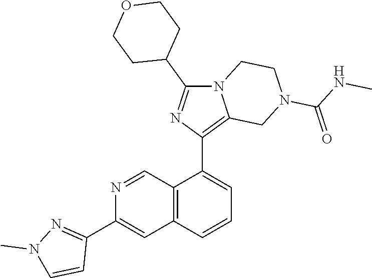 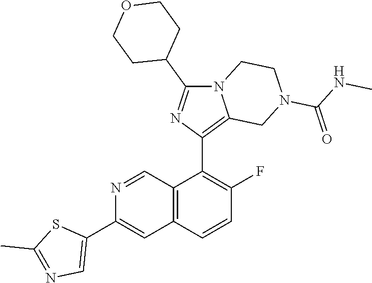 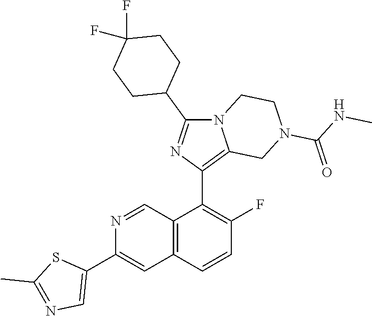 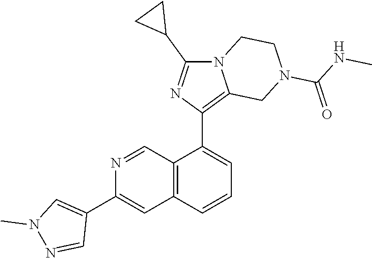
or a pharmaceutically acceptable salt thereof.

64. The pharmaceutical composition of claim 31, wherein the low molecular weight compound is a compound represented by formula (18) 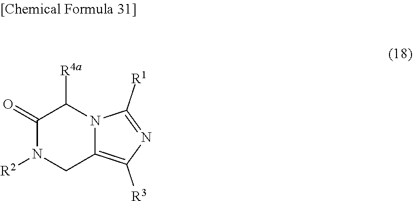
wherein
R¹ is hydrogen, or selected from alkyl, amino, alkoxy, heteroalkyl, cycloalkyl, heterocycloalkyl, halogen, haloalkyl, sulfonylalkyl, aryl, and heteroaryl, which is optionally substituted with 1, 2 or 3 group R⁵;
R² is hydrogen, or selected from alkyl, heteroalkyl, cycloalkyl, heterocycloalkyl, and haloalkyl, which is optionally substituted with 1, 2, or 3 group R⁶;
R³ is selected from alkyl, amino, alkoxy, heteroalkyl, cycloalkyl, heterocycloalkyl, alkylcarbonyl, alkylsulfonyl, arylcarbonyl, arylsulfonyl, aryl, and heteroaryl, wherein the R³ is:
(a) optionally substituted with 1, 2, or 3 group R⁷, and
(b) optionally substituted with one R⁸;
R^{4a} is selected from hydrogen, halogen, alkyl, heteroalkyl, cycloalkyl, and heterocycloalkyl, wherein the R^{4a} is optionally substituted with 1, 2 or 3 group R⁹;
R⁵ is each independently selected from alkyl, alkoxy, alkoxyalkyl, alkylcarbonyl, alkylsulfonyl, amino, aminocarbonyl, cyano, carboxy, halogen, haloalkoxy, haloalkyl, hydroxy, hydroxyalkyl, and oxo;
R⁶ and R⁷ are each independently selected from alkyl, alkoxy, cyano, carboxy, halogen, haloalkyl, hydroxy, and oxo;
R⁸ is selected from heterocycloalkyl, aryl, and heteroaryl, wherein the R⁸ is optionally substituted with 1, 2, or 3 group R¹⁰;
R⁹ is each independently selected from alkyl, alkoxy, cyano, carboxy, halogen, haloalkyl, hydroxy, and oxo;
R¹⁰ is each independently selected from alkyl, alkoxy, cyano, carboxy, halogen, haloalkyl, and hydroxy,
or a prodrug thereof or a pharmaceutically acceptable salt thereof.

65. The pharmaceutical composition of claim 31, wherein the low molecular weight compound is a compound presented by the following (Table 16)
TABLE 16 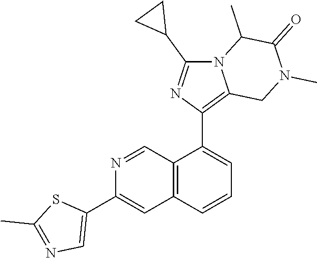 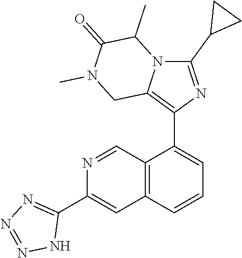 [SMILES: CC1C(=O)N(C)Cc2c(-c3cccc4cc(-c5nnn[nH]5)ncc34)nc(C3CC3)n21] 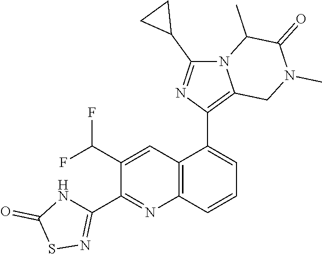 [SMILES: CC1C(=O)N(C)Cc2c(-c3cccc4nc(-c5nsc(=O)[nH]5)c(C(F)F)cc34)nc(C3CC3)n21] 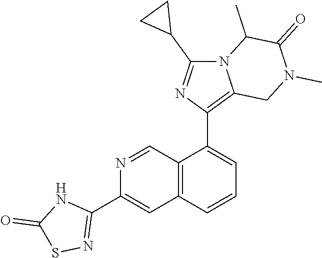 [SMILES: CC1C(=O)N(C)Cc2c(-c3cccc4cc(-c5nsc(=O)[nH]5)ncc34)nc(C3CC3)n21] 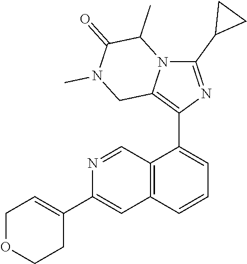 [SMILES: CC1C(=O)N(C)Cc2c(-c3cccc4cc(C5=CCOCC5)ncc34)nc(C3CC3)n21] 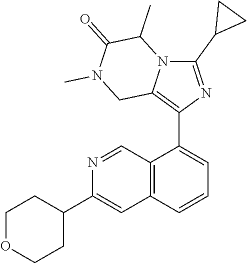 [SMILES: CC1C(=O)N(C)Cc2c(-c3cccc4cc(C5CCOCC5)ncc34)nc(C3CC3)n21] 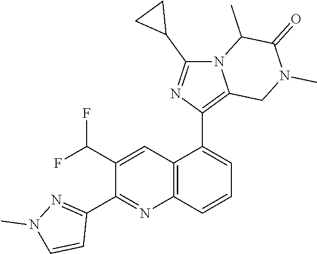 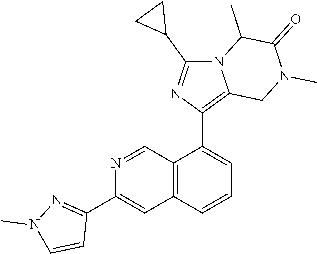 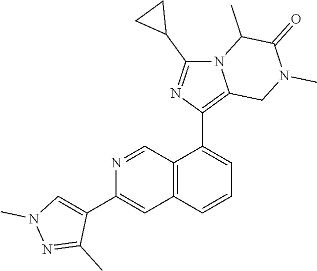 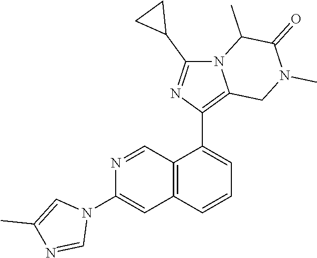 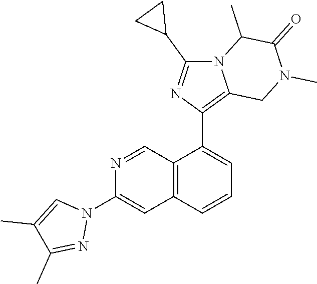 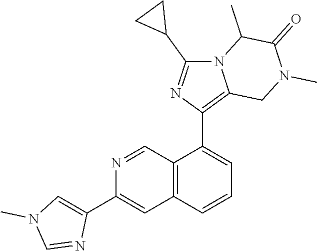 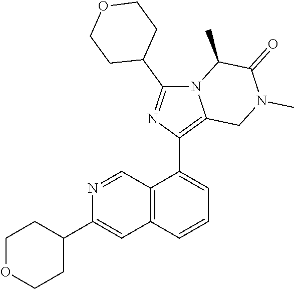 [SMILES: C[C@H]1C(=O)N(C)Cc2c(-c3cccc4cc(C5CCOCC5)ncc34)nc(C3CCOCC3)n21] 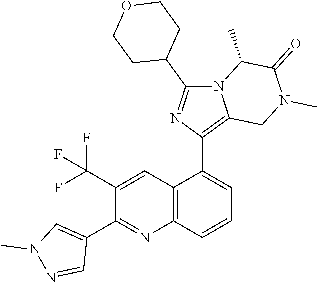 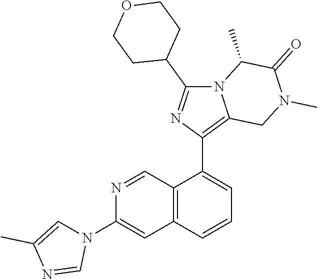 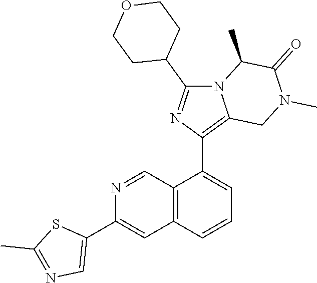 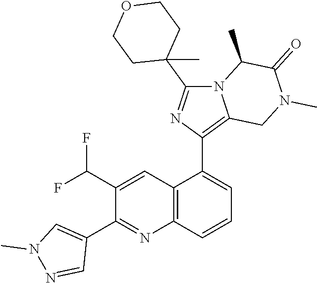 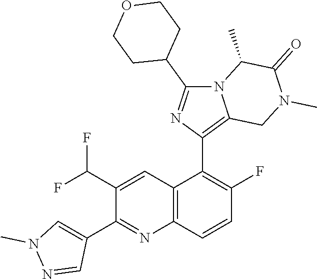 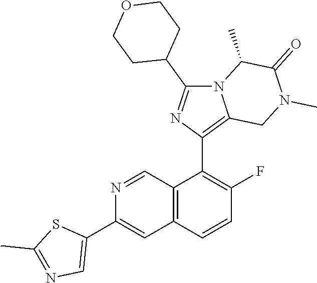 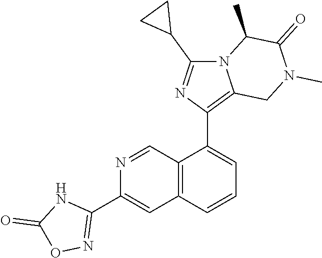 [SMILES: C[C@H]1C(=O)N(C)Cc2c(-c3cccc4cc(-c5noc(=O)[nH]5)ncc34)nc(C3CC3)n21] 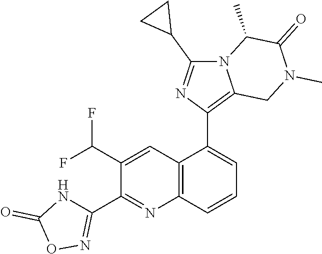 [SMILES: C[C@@H]1C(=O)N(C)Cc2c(-c3cccc4nc(-c5noc(=O)[nH]5)c(C(F)F)cc34)nc(C3CC3)n21] 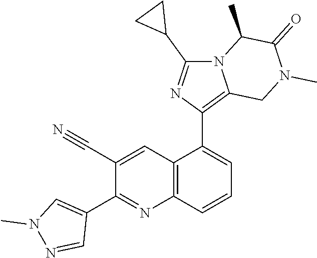
or a pharmaceutically acceptable salt thereof.

66. The pharmaceutical composition of claim 31, wherein the low molecular weight compound is a compound represented by formula (19) 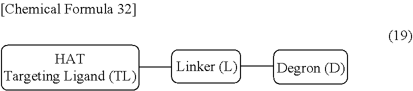
wherein
Targeting Ligand (TL) represents a structure that attaches to P300, Degron (D) represents a structure that attaches to an E3 ubiquitin ligase, and Linker (L) represents a structure that covalently attaches to Degron and Targeting Ligand,
or a prodrug thereof or a pharmaceutically acceptable salt thereof.

67. The pharmaceutical composition of claim 31, wherein the low molecular weight compound is a compound represented by
TABLE 17 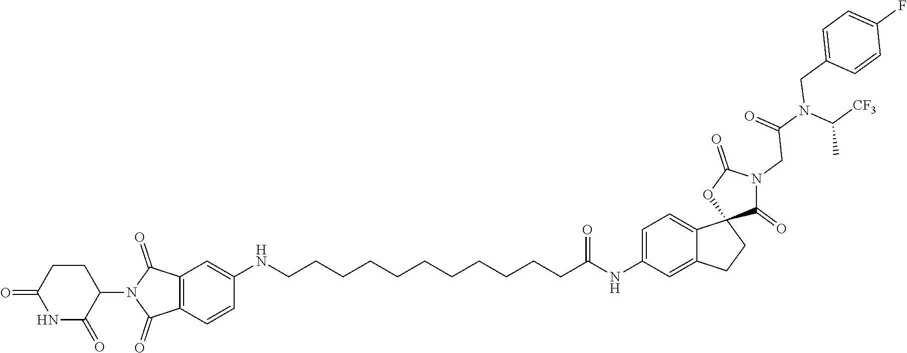 [SMILES: C[C@H](N(Cc1ccc(F)cc1)C(=O)CN1C(=O)O[C@@]2(CCc3cc(NC(=O)CCCCCCCCCCCNc4ccc5c(c4)C(=O)N(C4CCC(=O)NC4=O)C5=O)ccc32)C1=O)C(F)(F)F] 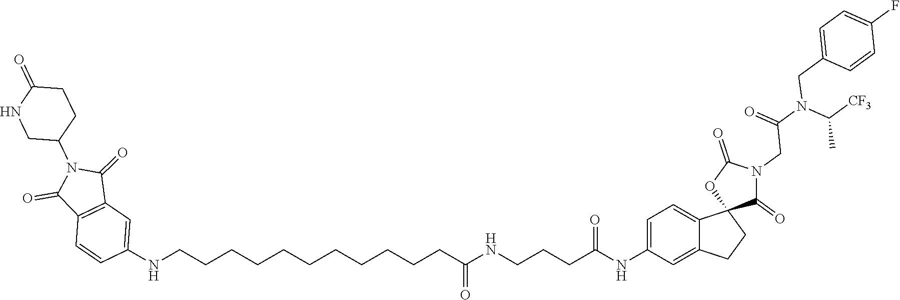 [SMILES: C[C@H](N(Cc1ccc(F)cc1)C(=O)CN1C(=O)O[C@@]2(CCc3cc(NC(=O)CCCNC(=O)CCCCCCCCCCCNc4ccc5c(c4)C(=O)N(C4CCC(=O)NC4)C5=O)ccc32)C1=O)C(F)(F)F] 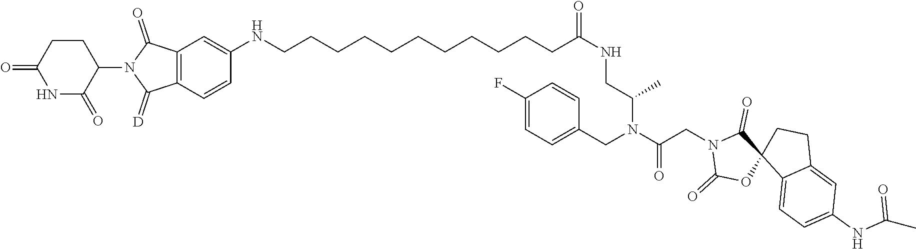
or a pharmaceutically acceptable salt thereof.

68. The pharmaceutical composition of claim 31, wherein the low molecular weight compound is a compound represented by formula (20) 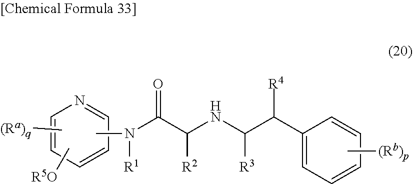
wherein
R¹, R³, and R⁴ are each independently hydrogen or C₁₋₄ alkyl;
R² is phenyl or 5- to 6-membered heteroaryl, each optionally substituted with 1 to 3 R^{c};
R⁵ is C₁₋₆ alkyl substituted with 4- to 6-membered heterocyclyl or 5- to 6-membered heteroaryl (wherein the heterocyclyl and the heteroaryl are optionally substituted with 1 to 3 R^{d}), 4- to 6-membered heterocyclyl (wherein the heterocyclyl is optionally substituted with 1 to 3 R^{d}), or 5- to 6-membered heteroaryl (wherein the heteroaryl is optionally substituted with 1 to 3 R^{d});
R^{a}, R^{b}, R^{c}, and R^{d} are each independently a halogen atom, CN, oxo, NO₂, C₁₋₆ alkyl, C₂₋₆ alkenyl, C₁₋₆ alkoxy, C₁₋₆ haloalkoxy, C₁₋₆ haloalkyl, —C₁₋₆ alkyl OR^{c}, —C(O)R^{f}, —C(O)OR, —C₁₋₆ alkyl C(O)OR^{e}, —C(O)N(R^{e})₂, —C(O)NR^{e}C₁₋₆ alkyl OR^{e}, —OC₁₋₆ alkyl N(R^{c})₂, —C₁₋₆ alkyl C(O)N(R^{c})₂, —C₁₋₆ alkyl N(R^{c})₂, —N(R^{e})₂, —C(O)NR^{e}C₁₋₆ alkyl N(R^{e})₂, —NR^{e}C₁₋₆ alkyl N(R^{e})₂, —NR^{e}C₁₋₆ alkyl OR^{e}, —SOR^{e}, —S(O)₂R^{e}, —SON(R^{e})₂, —SO₂N(R^{e})₂, —O(C₃₋₆) cycloalkyl, —O—C₁₋₄ alkyl-aryl, —C₁₋₆ alkyl(C₃₋₆) cycloalkyl, —C₁₋₆ alkylaryl, —C₁₋₆ alkylheteroaryl, —C₁₋₆ alkylheterocyclyl, C₃₋₆ cycloalkyl, heterocyclyl, heteroaryl, or aryl (wherein each of them, alone or attached to —O(C₃₋₆)cycloalkyl, —C₁₋₆ alkyl(C₁₋₆) cycloalkyl, —C₁₋₆ alkylaryl, —C₁₋₆ alkylheteroaryl, or —C₁₋₆ alkylheterocyclyl, is optionally substituted with 1 to 3 groups selected from halogen, C₁₋₆ alkyl, C₁₋₆ haloalkyl, C₁₋₆ alkoxy, C₁₋₆ haloalkoxy, —N(R^{e})₂, —C(O)R^{f}, and —C₁₋₆ alkyl OR^{e});
each R^{e} is hydrogen, C₁₋₄ haloalkyl, or C₁₋₄ alkyl,
each R^{f} is hydrogen, C₁₋₄ haloalkyl, C₁₋₄ alkyl, or C₃₋₄ cycloalkyl,
q is 0, 1, or 2, and
p is 0, 1, 2, or 3,
or a prodrug thereof or a pharmaceutically acceptable salt thereof.

69. The pharmaceutical composition of claim 31, wherein the low molecular weight compound is a compound represented by
TABLE 18 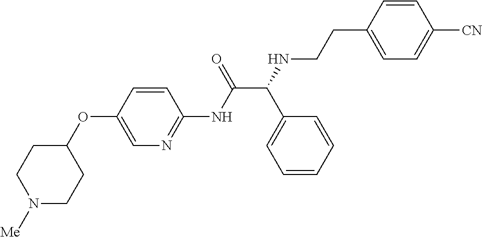 [SMILES: CN1CCC(Oc2ccc(NC(=O)[C@H](NCCc3ccc(C#N)cc3)c3ccccc3)nc2)CC1] 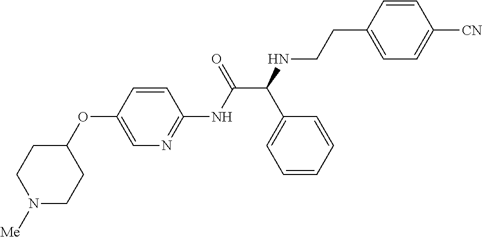 [SMILES: CN1CCC(Oc2ccc(NC(=O)[C@@H](NCCc3ccc(C#N)cc3)c3ccccc3)nc2)CC1] 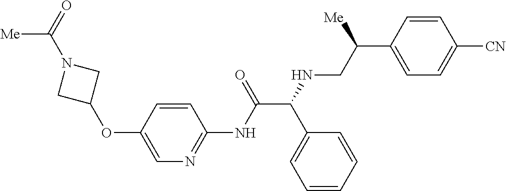 [SMILES: CC(=O)N1CC(Oc2ccc(NC(=O)[C@H](NC[C@@H](C)c3ccc(C#N)cc3)c3ccccc3)nc2)C1] 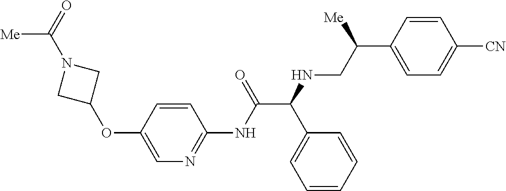 [SMILES: CC(=O)N1CC(Oc2ccc(NC(=O)[C@@H](NC[C@@H](C)c3ccc(C#N)cc3)c3ccccc3)nc2)C1] 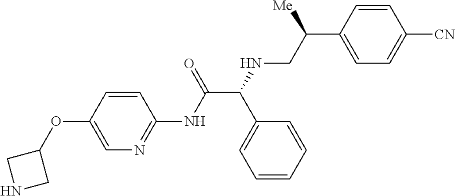 [SMILES: C[C@H](CN[C@@H](C(=O)Nc1ccc(OC2CNC2)cn1)c1ccccc1)c1ccc(C#N)cc1] 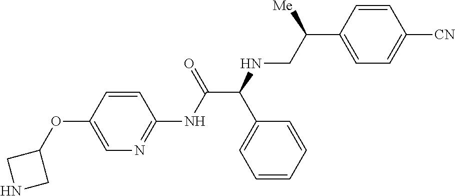 [SMILES: C[C@H](CN[C@H](C(=O)Nc1ccc(OC2CNC2)cn1)c1ccccc1)c1ccc(C#N)cc1] 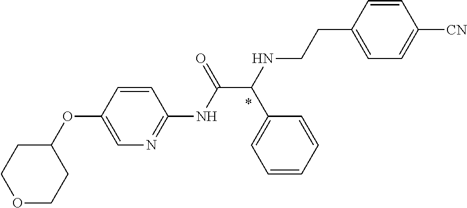 [SMILES: N#Cc1ccc(CCNC(C(=O)Nc2ccc(OC3CCOCC3)cn2)c2ccccc2)cc1] 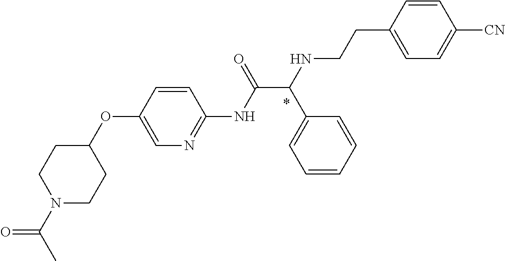 [SMILES: CC(=O)N1CCC(Oc2ccc(NC(=O)C(NCCc3ccc(C#N)cc3)c3ccccc3)nc2)CC1] 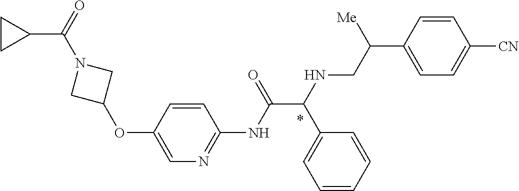 [SMILES: CC(CNC(C(=O)Nc1ccc(OC2CN(C(=O)C3CC3)C2)cn1)c1ccccc1)c1ccc(C#N)cc1] 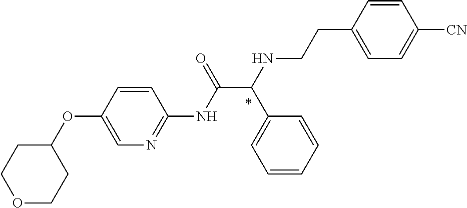 [SMILES: N#Cc1ccc(CCNC(C(=O)Nc2ccc(OC3CCOCC3)cn2)c2ccccc2)cc1] 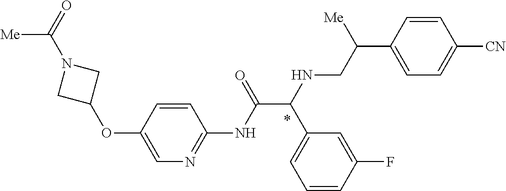 [SMILES: CC(=O)N1CC(Oc2ccc(NC(=O)C(NCC(C)c3ccc(C#N)cc3)c3cccc(F)c3)nc2)C1] 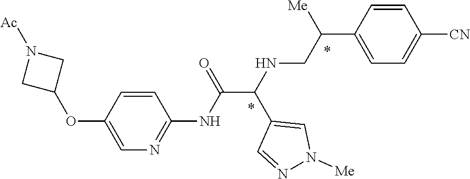 [SMILES: CC(=O)N1CC(Oc2ccc(NC(=O)C(NCC(C)c3ccc(C#N)cc3)c3cnn(C)c3)nc2)C1]
or a pharmaceutically acceptable salt thereof.

70. The pharmaceutical composition of claim 31, wherein the low molecular weight compound is a compound represented by formula (21) 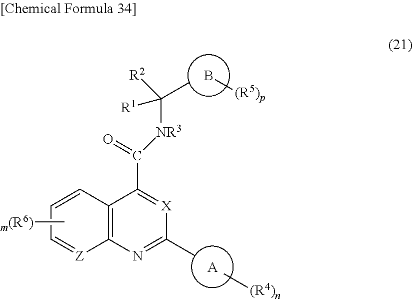
wherein
X is CH or N;
Z is N, CH, or CR⁶;
ring A is monocyclic aryl, bicyclic aryl, monocyclic heterocyclyl, or bicyclic heterocyclyl;
ring B is 5-membered N-containing heteroaryl;
R¹ and R² are each independently hydrogen, C₁₋₆ alkyl, a halogen atom, CN, —C(O)R^{1a}, —C(O)OR^{1a}, —C(O)N(R^{1a})₂, —N(R^{1a}), —N(R^{1a})C(O)R^{1a}, —N(R^{1a})C(O)OR^{1a}, —N(R^{1a})C(O)N(R^{1a})₂, —N(R^{1a})S(O)OR^{1a}, —OR^{1a}—, —OC(O)R^{1a}, —OC(O)N(R^{1a}), —SR^{1a}, —S(O)R^{1a}, —S(O)₂R^{1a}, —S(O)N(R^{1a})₂, or —S(O)₂N(R^{1a})₂;
R^{1a} is each independently hydrogen, C₁₋₆ alkyl, C₂₋₆ alkenyl, C₂₋₆ alkynyl, carbocyclyl, or heterocyclyl, or two R^{1a}, together with the nitrogen atom to which they are attached, may form a 4- to 7-membered ring (wherein the 4- to 7-membered ring may contain 1 to 2 each independently selected nitrogen atoms, oxygen atoms, or sulfur atoms);
R³ is hydrogen or C₁₋₆ alkyl;
R⁴ is each independently C₁₋₆ alkyl, C₂₋₆ alkenyl, C₂₋₆ alkynyl, carbocyclyl, heterocyclyl, a halogen atom, CN, —C(O)R^{4a}, —C(O)OR^{4a}, —C(O)N(R^{4a})₂, —N(R^{4a})₂, —N(R^{4a})C(O)R^{4a}, —N(R^{4a})C(O)OR^{4a}, —N(R^{4a})C(O)N(R^{4a})₂, —N(R^{4a})S(O)OR^{4a}, —OR^{4a}, —OC(O)R^{4a}, —OC(O)N(R^{4a})₂, —SR^{4a}, —S(O)R^{4a}, —S(O)₂R^{4a}, —S(O)N(R^{a})₂, —S(O)₂N(R^{4a})₂, or —P(O)(R^{4a})
R^{4a} is each independently hydrogen, C₁₋₆ alkyl, C₂₋₆ alkenyl, C₂₋₆ alkynyl, carbocyclyl, heterocyclyl, or —P(O)(R^{7a})₂, or two R^{4a}, together with the nitrogen atom to which they are attached, may form a 4- to 7-membered ring (wherein the groups may contain 1 to 2 each independently selected nitrogen atoms, oxygen atoms, or sulfur atoms);
R⁵ is each independently C₁₋₆ alkyl or carbocyclyl, or wherein two R⁵, together with the atom to which they are attached, may form a 4- to 7-membered ring (wherein the 4- to 7-membered ring may contain 1 to 2 each independently selected nitrogen atoms, oxygen atoms, or sulfur atoms);
R⁶ is each independently C₁₋₆ alkyl, C₂₋₆ alkenyl, C₂₋₆ alkynyl, carbocyclyl, heterocyclyl, a halogen atom, —CN, —C(O)R^{6a}, —C(O)OR^{6a}, —C(O)N(R^{6a})₂, —N(R^{6a})₂, —N(R^{6a})C(O)R^{6a}, —N(R^{6a})C(O)OR^{6a}, —N(R^{6a})C(O)N(R^{6a})₂, —N(R^{6a})S(O)OR^{6a}, —OR^{6a}, —OC(O)R^{6a}, —OC(O)N(R^{6a})₂, —SR^{6a}, —S(O)R^{6a}, —S(O)₂R^{6a}, —S(O)N(R^{6a})₂, —S(O)₂N(R^{6a})₂, or —P(O)(R^{6a})₂;
R^{6a} is each independently hydrogen, C₁₋₆ alkyl, C₂₋₆ alkenyl, C₂₋₆ alkynyl, carbocyclyl, or heterocyclyl, or two R^{6a}, together with the nitrogen atom to which they are attached, may form a 4- to 7-membered ring (wherein the 4- to 7-membered ring may contain 1 to 2 each independently selected nitrogen atoms, oxygen atoms, or sulfur atoms);
m is 0, 1, 2, or 3;
p is 0, 1, 2, or 3;
n is 0, 1, 2, 3, 4, 5, or 6;
the C₁₋₆ alkyl, C₂₋₆ alkenyl, C₂₋₆ alkynyl, carbocyclyl, and heterocyclyl is optionally substituted with one or two or more independent R⁷, halogen atom, —CN, —C(O)R⁷, —C(O)OR⁷, —C(O)N(R⁷)₂, —N(R⁷)₂, —N(R⁷)C(O)R⁷, —N(R⁷)C(O)OR⁷, —N(R⁷)C(O)N(R⁷)₂, —N(R⁷) S(O) OR⁷, —OR⁷, —OC(O)R⁷, —OC(O)N(R⁷)₂, —SR⁷, —S(O)₂R⁷, —S(O)₂R⁷, —S(O)₂N(R⁷)₂, —S(O)₂N(R⁷)₂, or —P(O)(R⁷)₂;
R⁷ is each independently hydrogen, C₁₋₆ alkyl, C₂₋₆ alkenyl, C₂₋₆ alkynyl, carbocyclyl, or heterocyclyl, wherein the C₁₋₆ alkyl, the C₂₋₆ alkenyl, the C₂₋₆ alkynyl, the carbocyclyl, or the heterocyclyl is optionally substituted with one or two or more substituents selected from R^{7a}, a halogen atom, —CN, —C(O)R^{7a}, —C(O)OR^{7a}, —C(O)N(R^{7a})₂, —N(R^{7a})₂, —N(R^{7a})C(O)R^{7a}, —N(R^{7a})C(O)OR^{7a}, —N(R^{7a})C(O)N(R^{7a})₂, —N(R^{7a})S(O)OR^{7a}, —OR^{7a}, —OC(O)R^{7a}, —OC(O)N(R^{7a})₂, —SR^{7a}, —S(O)R^{7a}, —S(O)₂R^{7a}, —S(O)₂N(R^{7a})₂, —S(O)₂N(R^{7a})₂, and —P(O)(R^{7a})₂; and
R^{7a} is each independently hydrogen or C₁₋₄ alkyl,
or a prodrug thereof or a pharmaceutically acceptable salt thereof.

71. The pharmaceutical composition of claim 31, wherein the low molecular weight compound is a compound represented by
TABLE 19 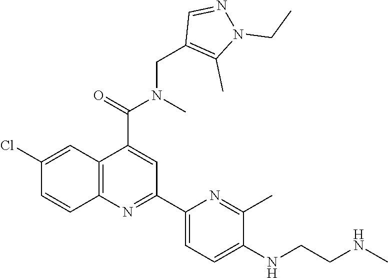 [SMILES: CCn1ncc(CN(C)C(=O)c2cc(-c3ccc(NCCNC)c(C)n3)nc3ccc(Cl)cc23)c1C] 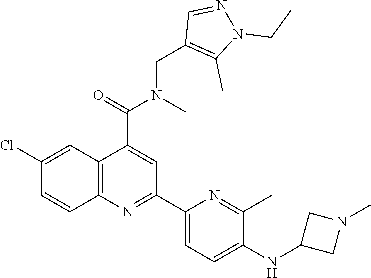 [SMILES: CCn1ncc(CN(C)C(=O)c2cc(-c3ccc(NC4CN(C)C4)c(C)n3)nc3ccc(Cl)cc23)c1C] 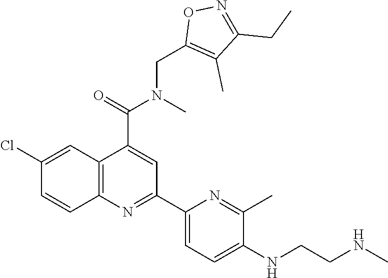 [SMILES: CCc1noc(CN(C)C(=O)c2cc(-c3ccc(NCCNC)c(C)n3)nc3ccc(Cl)cc23)c1C] 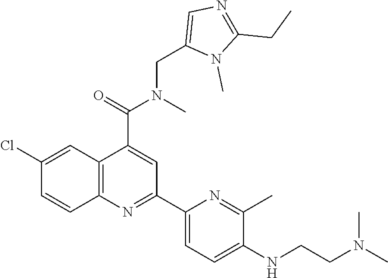 [SMILES: CCc1ncc(CN(C)C(=O)c2cc(-c3ccc(NCCN(C)C)c(C)n3)nc3ccc(Cl)cc23)n1C]
or a pharmaceutically acceptable salt thereof.

72. The pharmaceutical composition of claim 31, wherein the low molecular weight compound is a compound represented by formula (22) 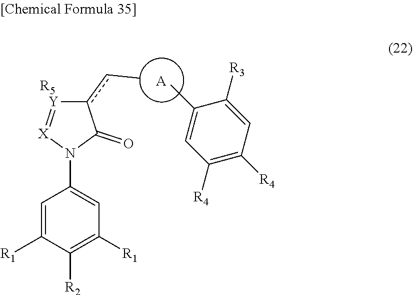
wherein
ring A is 5- or 6-membered aryl, or heteroaryl comprising a nitrogen, oxygen, or sulfur atom, and 1 to 4 carbons;
R¹ is hydrogen or halogen;
R² is a hydroxyl group, carboxyl, C₁₋₄ sulfoalkyl, boronic acid, or nitrogen-containing 5-membered heteroaryl;
R³ is trifluoromethyl, trifluoromethoxy, phosphinyl, nitro, difluoromethyl, or cyclopentanone-containing carbocyclyl;
R⁴ is hydrogen or methyl;
R⁵ is hydrogen, C₁₋₄ alkyl, or cycloalkyl;
X is —C(O)— or —N═;
Y is a carbon atom, a sulfur atom, or —NH—,
if X is —N═, Y is a carbon atom, and there is a double bond between X and Y, and if X is —C(O)—, Y is a sulfur atom or —NH—, and there is a single bond between X and Y, but an R⁵ group is absent,
or a prodrug thereof or a pharmaceutically acceptable salt thereof.

73. The pharmaceutical composition of claim 31, wherein the low molecular weight compound is a compound represented by
TABLE 20 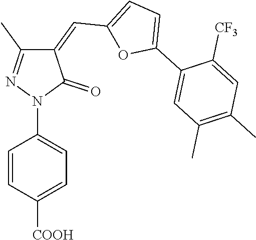 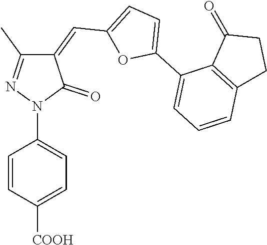 [SMILES: CC1=NN(c2ccc(C(=O)O)cc2)C(=O)/C1=C\c1ccc(-c2cccc3c2C(=O)CC3)o1] 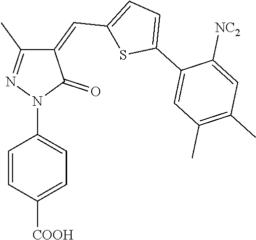
or a pharmaceutically acceptable salt thereof.

74. The pharmaceutical composition of claim 31, wherein the low molecular weight compound is a compound represented by formula (23) 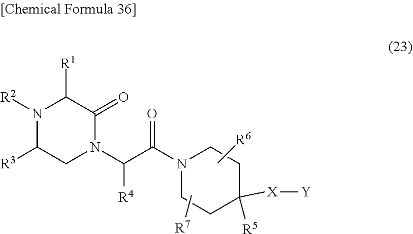
wherein
R¹ is hydrogen, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with cycloalkyl, aryl, or heteroaryl (wherein the cycloalkyl, the aryl, or the heteroaryl is optionally substituted with halogen, C₁₋₄ alkyl, or C₃₋₅ cycloalkyl);
R² is each independently hydrogen, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkylene-O—C₁₋₃ alkylene-O—C₁₋₃ alkylene, C₁₋₅ alkyl-NHCOR¹³, or C₁₋₃ alkyl substituted with cycloalkyl, aryl, or heteroaryl (wherein the cycloalkyl, the aryl, or the heteroaryl is optionally substituted with a halogen atom, C₁₋₄ alkyl, or C₃₋₅ cycloalkyl); with the proviso that if R² is C(O)NR¹⁵R¹⁵, both R¹⁵ may form a ring comprising a nitrogen atom of NR¹⁵R¹⁵ (wherein the ring may further comprise a heteroatom selected from an oxygen atom and a nitrogen atom, and if a nitrogen atom is contained, it is optionally substituted with R⁸);
R³ and R⁷ are each independently hydrogen, C₁₋₇ alkyl, C₂₋₇ alkenyl, C₂₋₇ alkynyl, C₃₋₇ cycloalkyl, or C₄₋₇ cycloalkenyl, which are optionally substituted with a halogen atom, OR⁸, NR⁸R¹¹, or C₁₋₃ alkyl substituted with aryl and heteroaryl (wherein the aryl and the heteroaryl are optionally substituted with a halogen atom, C₁₋₄ alkyl, or C₃₋₅ heteroalkyl);
R⁴ is C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with cycloalkyl, aryl, or heteroaryl (wherein the cycloalkyl, the aryl, or the heteroaryl is optionally substituted with a halogen atom, C₁₋₄ alkyl, or C₃₋₅ heteroalkyl);
R⁵ is hydrogen, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸, wherein R⁵, together with X and Y, may form a ring that may comprise a carbonyl group;
R⁶ is hydrogen, C₁₋₇ alkyl, C₂₋₇ alkenyl, C₂₋₇ alkynyl, C₃₋₇ cycloalkyl, C₄₋₇ cycloalkenyl (wherein the C₁₋₇ alkyl, the C₂₋₇ alkenyl, the C₂₋₇ alkynyl, the C₃₋₇ cycloalkyl, or the C₄₋₇ cycloalkenyl is optionally substituted with a halogen atom, OR⁸, NR⁸R¹¹, C₁₋₃ alkyl substituted with C(O)NR⁸R¹¹, or C₁₋₃ alkyl substituted with aryl or heteroaryl (wherein the aryl or the heteroaryl is optionally substituted with a halogen atom, C₁₋₄ alkyl, or C₃₋₅ cycloalkyl), wherein R⁶ may form a ring with any part of X, or is imidazolidinone;
R⁸ and R¹¹ are each independently hydrogen, C₁₋₇ alkyl, C₂₋₇ alkenyl, C₂₋₇ alkynyl, C₃₋₇ cycloalkyl, or C₄₋₇ cycloalkenyl;
X is a bond, C₁₋₇ alkylene, C₂₋₇ alkenylene, C₂₋₇ alkynylene, C₃₋₉ cycloalkylene, C₄₋₆ cycloalkenylene, —O—, C₁₋₃ alkylene-O—, —O—C₁₋₇ alkylene, —O—C₃₋₉ cycloalkylene, C₁₋₃ alkylene-O—C₁₋₇ alkylene, C₁₋₇ heteroalkylene, or —S—C₁₋₇ alkylene, wherein X, together with R¹, R⁶, and Y, may form a polycyclic system or a ring that may comprise a carbonyl group;
Y is hydrogen, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1, or 2, SO₂NR¹⁰R¹², NR¹⁰SO₂R¹⁰, NR¹⁰R¹², HNCOR⁸, CN, C₃₋₇ cycloalkyl that may comprise a nitrogen atom optionally substituted with R⁸ or an oxygen atom within a ring, S-aryl, O-aryl, S-heteroaryl, O-heteroaryl (wherein the S-aryl, the O-aryl, the S-heteroaryl, or the O-heteroaryl is optionally substituted with one or two or more R⁹ or R¹⁴), aryl, or heteroaryl (wherein the aryl or the heteroaryl is optionally substituted with one or two or more R⁸); wherein Y may form a ring that may comprise a carbonyl group at any position on X or R⁵, but if Y is C(O)NR¹⁰R¹² or NR¹⁰R¹², R¹⁰ and R¹² may form a ring comprising a nitrogen atom of NR¹⁰R¹² (wherein the ring may further comprise a heteroatom selected from an oxygen atom and a nitrogen atom, and if a nitrogen atom is contained, it is optionally substituted with R⁸);
R⁹ is hydrogen, a halogen atom, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR₈C(O)NR⁸R¹¹, OC(O)NR⁸R¹¹, SO₂NR⁸R¹¹, NR⁸SO₂R⁸, OR⁸, NR⁸R¹¹, or S(O)nR⁸ wherein n is 0, 1, or 2;
R¹⁰ and R¹² are each independently hydrogen, C₁₋₇ alkyl, C₂₋₇ alkenyl, C₂₋₇ alkynyl, C₃₋₇ cycloalkyl, or C₄₋₇ cycloalkenyl, C₁₋₃ alkylene-O—C₁₋₃ alkylene-O—C₁₋₃ alkylene, C₁₋₃ alkyl-aryl, or C₁₋₃ alkyl-heteroaryl, wherein R¹⁰ and R¹² are optionally substituted with a halogen atom, OR₈, or NR₈R¹¹;
R¹³ is C₁₋₇ alkyl substituted with a bicycle that may comprise at least one heteroatom or carbonyl group;
R¹⁴ is hydrogen, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted with aryl or heteroaryl (wherein the aryl or the heteroaryl is optionally substituted with a halogen atom, C₁₋₄ alkyl, or C₃₋₅ heteroalkyl); and
R¹⁵ is each independently hydrogen, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸,
or a prodrug thereof or a pharmaceutically acceptable salt thereof.

75. The pharmaceutical composition of claim 31, wherein the low molecular weight compound is a compound represented by
TABLE 21 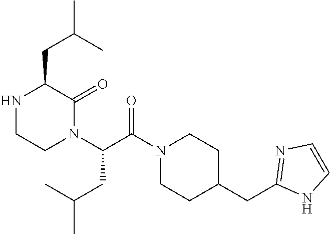 [SMILES: CC(C)C[C@@H]1NCCN([C@@H](CC(C)C)C(=O)N2CCC(Cc3ncc[nH]3)CC2)C1=O] 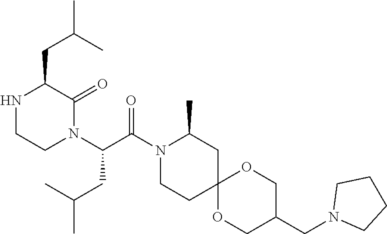 [SMILES: CC(C)C[C@@H]1NCCN([C@@H](CC(C)C)C(=O)N2CCC3(C[C@@H]2C)OCC(CN2CCCC2)CO3)C1=O] 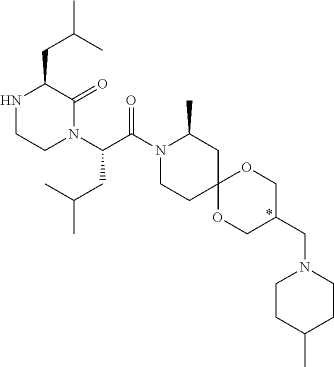 [SMILES: CC(C)C[C@@H]1NCCN([C@@H](CC(C)C)C(=O)N2CCC3(C[C@@H]2C)OCC(CN2CCC(C)CC2)CO3)C1=O] 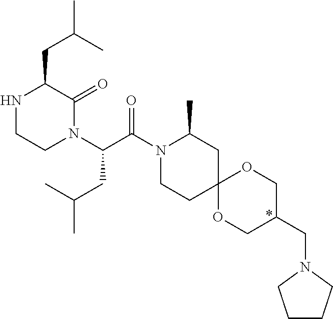 [SMILES: CC(C)C[C@@H]1NCCN([C@@H](CC(C)C)C(=O)N2CCC3(C[C@@H]2C)OCC(CN2CCCC2)CO3)C1=O] 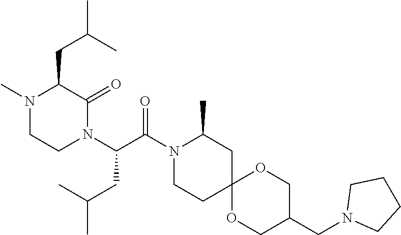 [SMILES: CC(C)C[C@H]1C(=O)N([C@@H](CC(C)C)C(=O)N2CCC3(C[C@@H]2C)OCC(CN2CCCC2)CO3)CCN1C] 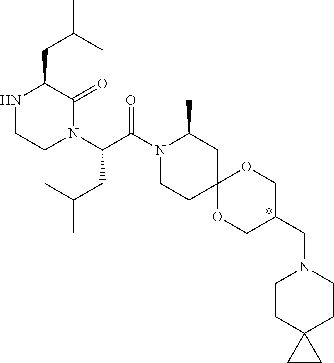 [SMILES: CC(C)C[C@@H]1NCCN([C@@H](CC(C)C)C(=O)N2CCC3(C[C@@H]2C)OCC(CN2CCC4(CC2)CC4)CO3)C1=O] 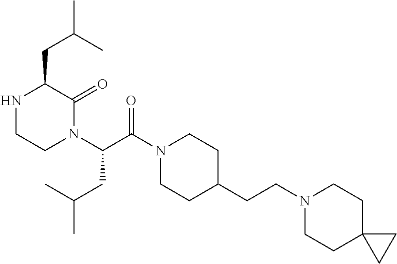 [SMILES: CC(C)C[C@@H]1NCCN([C@@H](CC(C)C)C(=O)N2CCC(CCN3CCC4(CC3)CC4)CC2)C1=O] 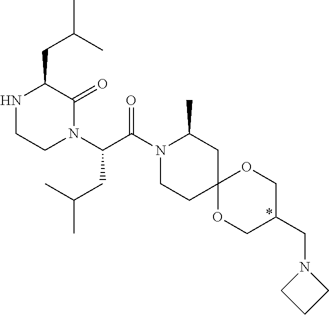 [SMILES: CC(C)C[C@@H]1NCCN([C@@H](CC(C)C)C(=O)N2CCC3(C[C@@H]2C)OCC(CN2CCC2)CO3)C1=O] 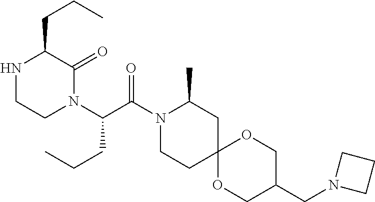 [SMILES: CCC[C@@H]1NCCN([C@@H](CCC)C(=O)N2CCC3(C[C@@H]2C)OCC(CN2CCC2)CO3)C1=O]
or a pharmaceutically acceptable salt thereof.

76. A pharmaceutical composition for use in treating and/or preventing cancer, comprising a CBP/P300 inhibitor as an active ingredient, characterized by being administered to a subject comprising at least one selected from the group consisting of a dysfunction of an SWI/SNF complex, and lack of or attenuation of expression of an SWI/SNF complex protein.

77. The pharmaceutical composition of claim 76, wherein the subject comprising at least one selected from the group consisting of a dysfunction of an SWI/SNF complex, and lack of or attenuation of expression of an SWI/SNF complex protein is determined by steps comprising
(1) a step comprising at least one selected from the group consisting of a step of detecting a mutation in an SWI/SNF complex gene of a cancer cell obtained from the subject, and a step of measuring expression of an SWI/SNF complex protein, and
(2) a step of determining that the subject comprises at least one selected from the group consisting of a dysfunction of an SWI/SNF complex, and lack of or attenuation of expression of an SWI/SNF complex protein based on at least one selected from the group consisting of the presence/absence of a mutation in an SWI/SNF complex gene and a result of expression of an SWI/SNF complex protein detected in (1).

78. The pharmaceutical composition of claim 77, wherein the SWI/SNF complex is a BAF complex, the SWI/SNF complex gene is a BAF complex gene, and the SWI/SNF complex protein is a BAF complex protein.

79. The pharmaceutical composition of claim 78, wherein
the BAF complex gene comprises at least one gene selected from the group consisting of an SMARC gene, an SS18-SSX fusion gene, and an ARID gene, and
the BAF complex protein comprises at least one protein selected from the group consisting of an SMARC protein, an SS18-SSX fusion protein, and an ARID protein.

80. The pharmaceutical composition of claim 78 or 79, wherein
the BAF complex gene is an SMARC gene, and
the BAF complex protein is an SMARC protein.

81. The pharmaceutical composition of claim 79 or 80, wherein
the SMARC gene comprises at least one gene selected from the group consisting of an SMARCB1 gene, an SMARCA2 gene, and an SMARCA4 gene, and
the SMARC protein comprises at least one protein selected from the group consisting of an SMARCB1 protein, an SMARCA2 protein, and an SMARCA4 protein.

82. The pharmaceutical composition of claim 79 or 80, wherein
the SMARC gene is an SMARCB1 gene, and
the SMARC protein is an SMARCB1 protein.

83. The pharmaceutical composition of claim 79 or 80, wherein the SMARC gene comprises an SMARCA2 gene and an SMARCA4 gene, and the SMARC protein comprises an SMARCA2 protein and an SMARCA4 protein.

84. The pharmaceutical composition of any one of claims 76 to 83, wherein the cancer is SMARC deficient cancer.

85. The pharmaceutical composition of claim 84, wherein the SMARC deficient cancer is SMARCB1 deficient cancer.

86. The pharmaceutical composition of claim 85, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

87. The pharmaceutical composition of claim 85, wherein the SMARCB1 deficient cancer is malignant rhabdoid tumor.

88. The pharmaceutical composition of claim 84, wherein the SMARC deficient cancer is SMARCA2/A4 deficient cancer.

89. The pharmaceutical composition of claim 88, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

90. The pharmaceutical composition of claim 88, wherein the SMARCA2/A4 deficient cancer is pulmonary adenocarcinoma.

91. The pharmaceutical composition of claim 78, wherein
the BAF complex gene is an ARID gene, and
the BAF complex protein is an ARID protein.

92. The pharmaceutical composition of claim 91, wherein
the ARID gene comprises at least one gene selected from the group consisting of an ARID1A gene and an ARID1B gene, and
the ARID protein comprises at least one protein selected from the group consisting of an ARID1A protein and an ARID1B protein.

93. The pharmaceutical composition of claim 91, wherein
the ARID gene is an ARID1A gene, and
the ARID protein is an ARID1A protein.

94. The pharmaceutical composition of claim 91, wherein the ARID gene is an ARID1A gene and an ARID1B gene, and the ARID protein is an ARID1A protein and an ARID1B protein.

95. The pharmaceutical composition of any one of claims 76 to 79 and 91 to 94, wherein the cancer is ARID deficient cancer.

96. The pharmaceutical composition of claim 95, wherein the ARID deficient cancer is ARID1A deficient cancer.

97. The pharmaceutical composition of claim 95, wherein the ARID1A deficient cancer comprises at least one selected from the group consisting of ovarian cancer, gastric cancer, bile duct cancer, pancreatic cancer, uterine cancer, neuroblastoma, colon cancer, and bladder cancer.

98. The pharmaceutical composition of claim 95, wherein the ARID1A deficient cancer is ovarian cancer.

99. The pharmaceutical composition of claim 95, wherein the ARID deficient cancer is ARID1A/1B deficient cancer.

100. The pharmaceutical composition of claim 99, wherein the ARID1A/1B deficient cancer comprises at least one selected from the group consisting of ovarian cancer, colon cancer, uterine cancer, neuroblastoma, bladder cancer, and gastric cancer.

101. The pharmaceutical composition of claim 99, wherein the ARID1A/1B deficient cancer is ovarian cancer.

102. The pharmaceutical composition of claim 78 or 79, wherein the BAF complex gene is an SS18-SSX fusion gene, and the BAF complex protein is an SS18-SSX fusion protein.

103. The pharmaceutical composition of any one of claims 76 to 79 and 102, wherein the cancer is SS18-SSX fusion cancer.

104. The pharmaceutical composition of claim 103, wherein the SS18-SSX fusion cancer is synovial sarcoma or Ewing's sarcoma.

105. The pharmaceutical composition of claim 103, wherein the SS18-SSX fusion cancer is synovial sarcoma.

106. The pharmaceutical composition of any one of claims 76 to 105, wherein the CBP/P300 inhibitor comprises at least one selected from the group consisting of reduction of expression of CBP and/or P300, and suppression of a function of CBP and/or P300.

107. The pharmaceutical composition of any one of claims 76 to 106, wherein the CBP/P300 inhibitor is a nucleic acid or a low molecular weight compound.

108. The pharmaceutical composition of any one of claims 76 to 107, wherein the CBP/P300 inhibitor is a low molecular weight compound.

109. A pharmaceutical composition comprising a CBP/P300 inhibitor in combination with at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, other antitumor agents, and agents classified as other antitumor agents.

110. A pharmaceutical composition comprising a CBP/P300 inhibitor for use in treating and/or preventing cancer by concomitantly using at least one agent selected from an anticancer alkylating agent, an anticancer antimetabolite, an anticancer antibiotic, a plant derived anticancer agent, an anticancer platinum coordination compound, an anticancer camptothecin derivative, an anticancer tyrosine kinase inhibitor, an anticancer serine-threonine kinase inhibitor, an anticancer phospholipid kinase inhibitor, a monoclonal antibody, an interferon, a biological response modifier, a hormone formulation, an angiogenesis inhibitor, an immune checkpoint inhibitor, an epigenetics-related molecule inhibitor, a post-translational protein modification inhibitor, a proteasome inhibitor, and agents classified as other antitumor agents.

111. A method for assisting prediction of efficacy of a CBP/P300 inhibitor on a subject, comprising at least one selected from the group consisting of detecting a dysfunction of an SWI/SNF complex in a cancer cell of the subject, and measuring expression of an SWI/SNF complex protein.

112. The method of claim 111, wherein the at least one selected from the group consisting of detecting a dysfunction of an SWI/SNF complex in a cancer cell, and measuring expression of an SWI/SNF complex protein is determined by steps comprising
(1) a step comprising at least one selected from the group consisting of detecting a mutation in an SWI/SNF complex gene of a cancer cell obtained from the subject and measuring expression of an SWI/SNF complex protein, and
(2) a step of determining that the subject comprises at least one selected from the group consisting of a dysfunction of an SWI/SNF complex, and lack of or attenuation of expression of an SWI/SNF complex protein based on at least one selected from the group consisting of the presence/absence of a mutation in an SWI/SNF complex gene and a result of expression of an SWI/SNF complex protein detected in (1).

113. A method of using at least one selected from the group consisting of the presence/absence or level of a mutation in an SWI/SNF complex gene and the presence/absence or level of expression of an SWI/SNF complex protein in a cancer cell of a subject as an indicator for predicting efficacy of a CBP/P300 inhibitor on the subject.

114. The method of claim 112 or 113, wherein the SWI/SNF complex is a BAF complex, the SWI/SNF complex gene is a BAF complex gene, and the SWI/SNF complex protein is a BAF complex protein.

115. The method of claim 114, wherein
the BAF complex gene comprises at least one gene selected from the group consisting of an SMARC gene, an SS18-SSX fusion gene, and an ARID gene, and
the BAF complex protein comprises at least one protein selected from the group consisting of an SMARC protein, an SS18-SSX fusion protein, and an ARID protein.

116. The method of claim 114 or 115, wherein
the BAF complex gene is an SMARC gene, and
the BAF complex protein is an SMARC protein.

117. The method of claim 115 or 116, wherein the SMARC gene comprises at least one gene selected from the group consisting of an SMARCB1 gene, an SMARCA2 gene, and an SMARCA4 gene, and the SMARC protein comprises at least one protein selected from the group consisting of an SMARCB1 protein, an SMARCA2 protein, and an SMARCA4 protein.

118. The method of claim 115 or 116, wherein the SMARC gene is an SMARCB1 gene, and the SMARC protein is an SMARCB1 protein.

119. The method of claim 115 or 116, wherein the SMARC gene comprises an SMARCA2 gene and an SMARCA4 gene, and the SMARC protein comprises an SMARCA2 protein and an SMARCA4 protein.

120. The method of any one of claims 111 to 119, wherein the cancer is SMARC deficient cancer.

121. The method of claim 120, wherein the SMARC deficient cancer is SMARCB1 deficient cancer.

122. The method of claim 121, wherein the SMARCB1 deficient cancer comprises at least one selected from the group consisting of malignant rhabdoid tumor, epithelioid sarcoma, atypical teratoid/rhabdoid tumor, nerve sheath tumor, chordoid meningioma, neuroepithelial tumor, glioneuronal tumor, craniopharyngioma, glioblastoma, chordoma, myoepithelial tumor, extraskeletal myxoid chondrosarcoma, synovial sarcoma, ossifying fibromyxoid tumor, basaloid squamous cell carcinoma of the paranasal cavity, esophageal adenocarcinoma, papillary thyroid cancer, follicular thyroid cancer, gastrointestinal stromal tumor, pancreatic undifferentiated rhabdoid tumor, digestive system rhabdoid tumor, renal medullary carcinoma, endometrial cancer, myoepithelioma-like tumor in the female vulvar region, colon cancer, and mesothelioma.

123. The method of claim 121, wherein the SMARCB1 deficient cancer is malignant rhabdoid tumor.

124. The method of claim 120, wherein the SMARC deficient cancer is SMARCA2/A4 deficient cancer.

125. The method of claim 124, wherein the SMARCA2/A4 deficient cancer comprises at least one selected from the group consisting of pulmonary adenocarcinoma, pleomorphic carcinoma, large cell lung carcinoma, esophageal cancer, gastroesophageal junction cancer, thoracic sarcoma, small cell carcinoma of the ovary, primary gallbladder tumor, uterine sarcoma, malignant rhabdoid tumor, ovarian granulosa tumor, adrenocortical cancer, and small cell lung cancer.

126. The method of claim 124, wherein the SMARCA2/A4 deficient cancer is pulmonary adenocarcinoma.

127. The method of claim 114 or 115, wherein
the BAF complex gene is an ARID gene, and
the BAF complex protein is an ARID protein.

128. The method of claim 127, wherein the ARID gene comprises at least one gene selected from the group consisting of an ARID1A gene and an ARID1B gene, and the ARID protein comprises at least one protein selected from the group consisting of an ARID1A protein and an ARID1B protein.

129. The method of claim 127, wherein the ARID gene is an ARID1A gene, and the ARID protein is an ARID1A protein.

130. The method of claim 127, wherein the ARID gene comprises an ARID1A gene and an ARID1B gene, and the ARID protein comprises an ARID1A protein and an ARID1B protein.

131. The method of any one of claims 111 to 115 and 127 to 130, wherein the cancer is ARID deficient cancer.

132. The method of claim 131, wherein the ARID deficient cancer is ARID1A deficient cancer.

133. The method of claim 132, wherein the ARID1A deficient cancer comprises at least one selected from the group consisting of ovarian cancer, gastric cancer, bile duct cancer, pancreatic cancer, uterine cancer, neuroblastoma, colon cancer, and bladder cancer.

134. The method of claim 132, wherein the ARID1A deficient cancer is ovarian cancer.

135. The method of claim 131, wherein the ARID deficient cancer is ARID1A/1B deficient cancer.

136. The method of claim 135, wherein the ARID1A/1B deficient cancer comprises at least one selected from the group consisting of ovarian cancer, colon cancer, uterine cancer, neuroblastoma, bladder cancer, and gastric cancer.

137. The method of claim 135, wherein the ARID1A/1B deficient cancer is ovarian cancer.

138. The method of claim 114 or 115, wherein the BAF complex gene is an SS18-SSX fusion gene, and the BAF complex protein is an SS18-SSX fusion gene protein.

139. The method of any one of claims 111 to 115 and 138, wherein the cancer is SS18-SSX fusion cancer.

140. The method of claim 139, wherein the SS18-SSX fusion cancer is synovial sarcoma or Ewing's sarcoma.

141. The method of claim 139, wherein the SS18-SSX fusion cancer is synovial sarcoma.

142. The method of any one of claims 111 to 141, wherein the CBP/P300 inhibitor comprises at least one selected from the group consisting of reduction of expression of CBP and/or P300, and suppression of a function of CBP and/or P300.

143. The method of any one of claims 111 to 142, wherein the CBP/P300 inhibitor is a nucleic acid or a low molecular weight compound.

144. The method of any one of claims 111 to 143, wherein the CBP/P300 inhibitor is a low molecular weight compound.


SEQUENCE LISTING (35 sequences)

SEQ ID NO: 1 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 2 - protein, 2404 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQAILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 3 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 4 - DNA, 10083 bp, Homo sapiens
ctgcggggcgctgttgctgtggctgagatttggccgccgcctcccccacccggcctgcgccctccctctccctcggcgcccgcccgcccgctcgcggcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccggctgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatccctgtaagaatgtttaaggtttttatttatttatatatattttttgagtctgttctttgtaagacatggttctggttgttcgctcatagcggagaggctggggctgcggttgtggttgtggcggcgtgggtggtggctgggaactgtggcccaggcttagcggccgcccggaggcttttcttcccggagactgaggtgggcgactgaggtgggcggctcagcgttggccccacacattcgaggctcacaggtgattgtcgctcacacagttagggtcgtcagttggtctgaaactgcatttggcccactcctccatcctccctgtccgtcgtagctgccacccccagaggcggcgcttcttcccgtgttcaggcggctccccccccccgtacacgactcccagaatctgaggcagagagtgctccaggctcgcgaggtgctttctgacttccccccaaatcctgccgctgccgcgcagcatgtcccgtgtggcgtttgaggaaatgctgagggacagacaccttggagcaccagctccggtccctgttacagtgagaaaggtcccccacttcgggggatacttgcacttagccacatggtcctgcctcccttggagtccagttccaggctcccttactgagtgggtgagacaagttcacaaaaaccgtaaaactgagaggaggaccatgggcaggggagctgaagttcatcccctaagtctaccacccccagcacccagagaacccactttatccctagtcccccaacaaaggctggtctaggtgggggtgatggtaattttagaaatcacgccccaaatagcttccgtttgggcccttacattcacagataggttttaaatagctgaatacttggtttgggaatctgaattcgaggaacctttctaagaagttggaaaggtccgatctagttttagcacagagctttgaaccttgagttataaaatgcagaataattcaagtaaaaataagaccaccatctggcacccctgaccagcccccattcaccccatcccaggaggggaagcacaggccgggcctccggtggagattgctgccactgctcggcctgctgggttcttaacctccagtgtcctcttcatcttttccacccgtagggaaaccttgagccatgtgttcaaacaagaagtggggctagagcccgagagcagcagctctaagcccacactcagaaagtggcgccctcctggttgtgcagccttttaatgtgggcagtggaggggcctctgtttcaggttatcctggaattcaaaacgttatgtaccaacctcatcctctttggagtctgcatcctgtgcaaccgtcttgggcaatccagatgtcgaaggatgtgaccgagagcatggtctgtggatgctaaccctaagtttgtcgtaaggaaatttctgtaagaaacctggaaagccccaacgctgtgtctcatgctgtatacttaagaggagaagaaaaagtcctatatttgtgatcaaaaagaggaaacttgaaatgtgatggtgtttataataaaagatggtaaaactacttggattcaaa

SEQ ID NO: 5 - DNA, 10790 bp, Homo sapiens
aattgaggaatcaacagccgccatcttgtcgcggacccgaccggggcttcgagcgcgatctactcggccccgccggtcccgggccccacaaccgcccgcgcaccccgctccgcccggccggcccgctccgcccggccctcggcgcccgccccggcggccccgctcgcctctcggctcggcctcccggagcccggcggcggcggcggcggcagcggcggcggcggcggcggaacggggggtgggggggccgcggcggcggcggcgaccccgctcggcgcattgtttttcctcacggcggcggcggcggcgggccgcgggccgggagcggagcccggagccccctcgtcgtcgggccgcgagcgaattcattaagtggggcgcggggggggagcgaggcggcggcggcggcggcaccatgttctcggggactgcctgagccgcccggccgggcgccgtcgctgccagccgggcccgggggggcggccgggccgccggggcgcccccaccgcggagtgtcgcgctcgggaggcgggcaggggatgagggggccgcggccggcggcggcggcggcggccgggggcgggcggtgagcgctgcggggcgctgttgctgtggctgagatttggccgccgcctcccccacccggcctgcgccctccctctccctcggcgcccgcccgcccgctcgcggcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccggctgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatccctgtaagaatgtttaaggtttttatttatttatatatattttttgagtctgttctttgtaagacatggttctggttgttcgctcatagcggagaggctggggctgcggttgtggttgtggcggcgtgggtggtggctgggaactgtggcccaggcttagcggccgcccggaggcttttcttcccggagactgaggtgggcgactgaggtgggcggctcagcgttggccccacacattcgaggctcacaggtgattgtcgctcacacagttagggtcgtcagttggtctgaaactgcatttggcccactcctccatcctccctgtccgtcgtagctgccacccccagaggcggcgcttcttcccgtgttcaggcggctccccccccccgtacacgactcccagaatctgaggcagagagtgctccaggctcgcgaggtgctttctgacttccccccaaatcctgccgctgccgcgcagcatgtcccgtgtggcgtttgaggaaatgctgagggacagacaccttggagcaccagctccggtccctgttacagtgagaaaggtcccccacttcgggggatacttgcacttagccacatggtcctgcctcccttggagtccagttccaggctcccttactgagtgggtgagacaagttcacaaaaaccgtaaaactgagaggaggaccatgggcaggggagctgaagttcatcccctaagtctaccacccccagcacccagagaacccactttatccctagtcccccaacaaaggctggtctaggtgggggtgatggtaattttagaaatcacgccccaaatagcttccgtttgggcccttacattcacagataggttttaaatagctgaatacttggtttgggaatctgaattcgaggaacctttctaagaagttggaaaggtccgatctagttttagcacagagctttgaaccttgagttataaaatgcagaataattcaagtaaaaataagaccaccatctggcacccctgaccagcccccattcaccccatcccaggaggggaagcacaggccgggcctccggtggagattgctgccactgctcggcctgctgggttcttaacctccagtgtcctcttcatcttttccacccgtagggaaaccttgagccatgtgttcaaacaagaagtggggctagagcccgagagcagcagctctaagcccacactcagaaagtggcgccctcctggttgtgcagccttttaatgtgggcagtggaggggcctctgtttcaggttatcctggaattcaaaacgttatgtaccaacctcatcctctttggagtctgcatcctgtgcaaccgtcttgggcaatccagatgtcgaaggatgtgaccgagagcatggtctgtggatgctaaccctaagtttgtcgtaaggaaatttctgtaagaaacctggaaagccccaacgctgtgtctcatgctgtatacttaagaggagaagaaaaagtcctatatttgtgatcaaaaagaggaaacttgaaatgtgatggtgtttataataaaagatggtaaaactacttggattcaaa

SEQ ID NO: 6 - DNA, 8701 bp, Homo sapiens
gagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctagtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

SEQ ID NO: 7 - DNA, 8779 bp, Homo sapiens
gagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

SEQ ID NO: 8 - DNA, 5191 bp, Homo sapiens
gccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatggtaaaaaaacaaaacctaacactaaggatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaggtcatgttcaatttcttcaaagttttaacataaaaataatgagagccaggagtggggccggggcctggggggacgaaggtggtatgtgaacaaggttggcacacaggcctcaccctcctctgcctcagattcccaagtgggcaggtgggggtgaatggggctccgggtagcacctcagctcctctcagctcccctcagcctgttctccttccagacccagagagctgagaagagtagctgtgaggctcagggcaagaggctctctgcctttcaggaacagccctaaccctgctccccttgcttggcctcaggaaggtgccgcgagctctcctgccgtccctgggccgccctggctctgctgtgtccagatggtcaggctactgccagctggggccttgctgctctgaagtcccctgcggagggcccagtcctgtgtgggcactgctgggctgtcgccagcctgggtgcaggagggctgttctagctccagtggcacccatagccaggtcagctggggccctttcccaccccagcaggtgctgtggcctgggccagctcctgccttacaagccagctgtgaggaatatgggaatagccctcccggcctggtgccagctcttggagttgacacggtacagggaggagacacagcccagggtcccttcccagccctgcctccaaggagttcatgtcccctctgttctcatctgtaatagggaggtgtccccattcttcagaatggacacaggatctgggagggcagcaaactggctcgcagctccagccttactgaagagaatgggcacagatccgggcacagatcccagcacagactgctgccaccctcagctgttggcaggtcccatgctgccagggcagggctagggtcagaggctgctgtgctccctggaagtggggtagggccccatgtggggcagaggcagagctctgattagggattggggttcttggtcgctgagatgtgagaggagggctcctttgagcacatgttagcatgggactcttcccagggagtttgcactcagggcctctgccctccatcaaagagtggaactccccagagccccatgcacagcaaggggacagctgggccttactggaaggccttgaacaaaggggaagattcccagcccagctgctcttagacatgaacaggtttcattgctgaggtgtttgttctgtccatgaggtaggaacctcggcaatgaaagggtgaggcagccctgtgtctccacaactggggggatggaaggaaccttggctgcctcaccccacaggtcgggcagggccacctggctgggaggtgccgggaaggctgggccctcactcctgaccgccagctcacaccgccgcaaagccatctccacaaggtctggctacaacacggagggcagactcaacagagaacagtgttgttaccatgaaaatgacaacctgtctttggaggaggccccgtgccactgagcatccagaaataaaccacaacatggacaggcttagaacaacaaggaaagctgccaggtcagaagagaaaaatgagccacaggggtcggataaggctcacacacgtcctcagctaaaaagggcaggaacagaaccttccagaagtccctgcctcacccagtctcagaactctgctaaggtgaaaacttaggctctgaggtcatagaaagggcagaagacctagtcctggccctcttctgcacctgaatccatggggctttggcatcaccagatgaaaaatgaggcatacgcccacctgtcagggtggctgatgagagacaggagaggctagattggcatcagcctgaaggcaccactggcaggaacatctgtaggctggtttggcacaacctaggagacgcctgtcctggccccagcagccgaaatctggtgaacttccccgctgactggcaggtagcagaggcctatggtgggcaggacttgcccaaggccctggtggggccaggatgagaaccctgagcctgtcacctgtgagctcaaaagctctgcctggcaacctgtgagctcaaagctctgccaggcaaccatgggcagtttctttgccctctgtgggcacccctatcctaccacctgcagttgggctgagaggccacactgagtgaggacggggcaggcatagaaggatgtggccaggtgagatggggaagccagtgctgtgggccaagagactgcagctcattctgtttattcaggtgggcccttgcatgggcccagcctttaggatgggttttttctgccccaagtaggggtcatgggtaggatggaagctgccagaagcctcttaggcctggccctgggtgggggtcactgctgcgggggtggcagatggggtcctggctgttcctcagggaggggcaggtaattggggtcttctgcaggggcatccaggagcagctttctgtggggaggggcccgtgttgagcacaggccagcacaggtccccatcggtggggatccttctgagggtggggagagggagggagggctctcaacactcacaggaagccaggggtctgcaggagcctcttgcctccaggctggttggggaagacgtcctccaggaagtagtagatatggcccaccgcaatccctgtgagacagccacggactgtggggtcaccctccacagcccagagtcctagaccagcagagcctgccccaggcccccatccacagcctggtggccctgcaggccccacagcatgagtgccccaaagccttgcacagagtgccagccccgggttggccgtgaaggacaagcttaaaaggcccagaagcaggcaggacccagggaggggagggcctgagaatagtggaggagtgggagccatggggcaggaaccctgaccctcccatcctcactcccatcaggaccgtgcaagcatcagtagatccgtcctgacgatgcaaattatgtgggccggctggcttgaggggctgtaagagcacagcagctgggagggcaggaagatggggatggagccaggtgtgaggagaactccagcaaggatgggagaggggccccagggcataagcagcgtgtcctgaggggagtggccagcctggggcggactagatgtaccgggaggctcacccagcaggtccacgaggatggagttgcccagcagcagcgagaagcccatgagcgcccaaggcaggaacggtgcctggaaagtgagcaggccgaagaagttgaccctcacccgagggctgcggcggctccacacgtacaccagcatggccatgagggcctggcccaggaagaacaggctgcccaggagtcccagcagctgggccagagtcaaggtgctccggtgcaggcctcagcccaagcccagggcccctctgacttcccaagaccctggaattcttcccctcatctcccctatgtgctattccctcatcaagatgagccagtccaataaaggcgacacactccacgggc

SEQ ID NO: 9 - DNA, 5164 bp, Homo sapiens
gccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatgatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaggtcatgttcaatttcttcaaagttttaacataaaaataatgagagccaggagtggggccggggcctggggggacgaaggtggtatgtgaacaaggttggcacacaggcctcaccctcctctgcctcagattcccaagtgggcaggtgggggtgaatggggctccgggtagcacctcagctcctctcagctcccctcagcctgttctccttccagacccagagagctgagaagagtagctgtgaggctcagggcaagaggctctctgcctttcaggaacagccctaaccctgctccccttgcttggcctcaggaaggtgccgcgagctctcctgccgtccctgggccgccctggctctgctgtgtccagatggtcaggctactgccagctggggccttgctgctctgaagtcccctgcggagggcccagtcctgtgtgggcactgctgggctgtcgccagcctgggtgcaggagggctgttctagctccagtggcacccatagccaggtcagctggggccctttcccaccccagcaggtgctgtggcctgggccagctcctgccttacaagccagctgtgaggaatatgggaatagccctcccggcctggtgccagctcttggagttgacacggtacagggaggagacacagcccagggtcccttcccagccctgcctccaaggagttcatgtcccctctgttctcatctgtaatagggaggtgtccccattcttcagaatggacacaggatctgggagggcagcaaactggctcgcagctccagccttactgaagagaatgggcacagatccgggcacagatcccagcacagactgctgccaccctcagctgttggcaggtcccatgctgccagggcagggctagggtcagaggctgctgtgctccctggaagtggggtagggccccatgtggggcagaggcagagctctgattagggattggggttcttggtcgctgagatgtgagaggagggctcctttgagcacatgttagcatgggactcttcccagggagtttgcactcagggcctctgccctccatcaaagagtggaactccccagagccccatgcacagcaaggggacagctgggccttactggaaggccttgaacaaaggggaagattcccagcccagctgctcttagacatgaacaggtttcattgctgaggtgtttgttctgtccatgaggtaggaacctcggcaatgaaagggtgaggcagccctgtgtctccacaactggggggatggaaggaaccttggctgcctcaccccacaggtcgggcagggccacctggctgggaggtgccgggaaggctgggccctcactcctgaccgccagctcacaccgccgcaaagccatctccacaaggtctggctacaacacggagggcagactcaacagagaacagtgttgttaccatgaaaatgacaacctgtctttggaggaggccccgtgccactgagcatccagaaataaaccacaacatggacaggcttagaacaacaaggaaagctgccaggtcagaagagaaaaatgagccacaggggtcggataaggctcacacacgtcctcagctaaaaagggcaggaacagaaccttccagaagtccctgcctcacccagtctcagaactctgctaaggtgaaaacttaggctctgaggtcatagaaagggcagaagacctagtcctggccctcttctgcacctgaatccatggggctttggcatcaccagatgaaaaatgaggcatacgcccacctgtcagggtggctgatgagagacaggagaggctagattggcatcagcctgaaggcaccactggcaggaacatctgtaggctggtttggcacaacctaggagacgcctgtcctggccccagcagccgaaatctggtgaacttccccgctgactggcaggtagcagaggcctatggtgggcaggacttgcccaaggccctggtggggccaggatgagaaccctgagcctgtcacctgtgagctcaaaagctctgcctggcaacctgtgagctcaaagctctgccaggcaaccatgggcagtttctttgccctctgtgggcacccctatcctaccacctgcagttgggctgagaggccacactgagtgaggacggggcaggcatagaaggatgtggccaggtgagatggggaagccagtgctgtgggccaagagactgcagctcattctgtttattcaggtgggcccttgcatgggcccagcctttaggatgggttttttctgccccaagtaggggtcatgggtaggatggaagctgccagaagcctcttaggcctggccctgggtgggggtcactgctgcgggggtggcagatggggtcctggctgttcctcagggaggggcaggtaattggggtcttctgcaggggcatccaggagcagctttctgtggggaggggcccgtgttgagcacaggccagcacaggtccccatcggtggggatccttctgagggtggggagagggagggagggctctcaacactcacaggaagccaggggtctgcaggagcctcttgcctccaggctggttggggaagacgtcctccaggaagtagtagatatggcccaccgcaatccctgtgagacagccacggactgtggggtcaccctccacagcccagagtcctagaccagcagagcctgccccaggcccccatccacagcctggtggccctgcaggccccacagcatgagtgccccaaagccttgcacagagtgccagccccgggttggccgtgaaggacaagcttaaaaggcccagaagcaggcaggacccagggaggggagggcctgagaatagtggaggagtgggagccatggggcaggaaccctgaccctcccatcctcactcccatcaggaccgtgcaagcatcagtagatccgtcctgacgatgcaaattatgtgggccggctggcttgaggggctgtaagagcacagcagctgggagggcaggaagatggggatggagccaggtgtgaggagaactccagcaaggatgggagaggggccccagggcataagcagcgtgtcctgaggggagtggccagcctggggcggactagatgtaccgggaggctcacccagcaggtccacgaggatggagttgcccagcagcagcgagaagcccatgagcgcccaaggcaggaacggtgcctggaaagtgagcaggccgaagaagttgaccctcacccgagggctgcggcggctccacacgtacaccagcatggccatgagggcctggcccaggaagaacaggctgcccaggagtcccagcagctgggccagagtcaaggtgctccggtgcaggcctcagcccaagcccagggcccctctgacttcccaagaccctggaattcttcccctcatctcccctatgtgctattccctcatcaagatgagccagtccaataaaggcgacacactccacgggc

SEQ ID NO: 10 - DNA, 5748 bp, Homo sapiens
gtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagataggtgtaacgtggagaaggtgcccagtaattctcagttggaaatagaaggaaacagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttga

SEQ ID NO: 11 - DNA, 5694 bp, Homo sapiens
gtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttga

SEQ ID NO: 12 - DNA, 5673 bp, Homo sapiens
gggcgcgcgcgcgaggcttcccctcgtttggcggcggcggcggcttctttgtttcgtgaagagaagcgagacgcccattctgcccccggccccgcgcggaggggcgggggaggcgccgggaagtcgacggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaaaattacaggaaaagatatccatgacacagccagcagtgtggcacgtgggctacaattccagcgtggccttcagttctgcacacgtgcgtcaaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaa

SEQ ID NO: 13 - DNA, 5653 bp, Homo sapiens
gggcgcgcgcgcgaggcttcccctcgtttggcggcggcggcggcttctttgtttcgtgaagagaagcgagacgcccattctgcccccggccccgcgcggaggggcgggggaggcgccgggaagtcgacggcgccggcggctcctgcgtctcgcccttttgcccaggctagagtgcagtggtgcggtcatggttcactgcagcctcaacctcctggactcagcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaa

SEQ ID NO: 14 - DNA, 8595 bp, Homo sapiens
ctcctttctccggcagcagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcagaattacaagcggccaatggatggcacatatggccctcctgccaagcggcacgaaggggagatgtacagcgtgccatacagcactgggcaggggcagcctcagcagcagcagttgcccccagcccagccccagcctgccagccagcaacaagctgcccagccttcccctcagcaagatgtatacaaccagtatggcaatgcctatcctgccactgccacagctgctactgagcgccgaccagcaggcggcccccagaaccaatttccattccagtttggccgagaccgtgtctctgcaccccctggcaccaatgcccagcaaaacatgccaccacaaatgatgggcggccccatacaggcatcagctgaggttgctcagcaaggcaccatgtggcaggggcgtaatgacatgacctataattatgccaacaggcagagcacgggctctgccccccagggccccgcctatcatggcgtgaaccgaacagatgaaatgctgcacacagatcagagggccaaccacgaaggctcgtggccttcccatggcacacgccagcccccatatggtccctctgcccctgtgccccccatgacaaggccccctccatctaactaccagcccccaccaagcatgcagaatcacattcctcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaa

SEQ ID NO: 15 - DNA, 7944 bp, Homo sapiens
ctcctttctccggcagcagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaa

SEQ ID NO: 16 - DNA, 7897 bp, Homo sapiens
acacatgcatagtgagattgctctacaaagggcagcttcccctcactgtctgtcagtaaaagaccaaaaggcatatgcaacatgttgtgatttaggttgaagtcttggcctcggaggatgcagcctttggactcaaggatctgtctggctccattgatgacctccccacgggaacggaagcaactttgagctcagcagtcagtgcatccgggtccacgagcagccaaggggatcagagcaacccggcgcagtcgcctttctccccacatgcgtcccctcatctctccagcatcccggggggcccatctccctctcctgttggctctcctgtaggaagcaaccagtctcgatctggcccaatctctcctgcaagtatcccaggtagtcagatgcctccgcagccacccgggagccagtcagaatccagttcccatcccgccttgagccagtcaccaatgccacaggaaagaggttttatggcaggcacacaaagaaaccctcagatggctcagtatggacctcaacagacaggaccatccatgtcgcctcatccttctcctgggggccagatgcatgctggaatcagtagctttcagcagagtaactcaagtgggacttacggtccacagatgagccagtatggaccacaaggtaactactccagacccccagcgtatagtggggtgcccagtgcaagctacagcggcccagggcccggtatgggtatcagtgccaacaaccagatgcatggacaagggccaagccagccatgtggtgctgtgcccctgggacgaatgccatcagctgggatgcagaacagaccatttcctggaaatatgagcagcatgacccccagttctcctggcatgtctcagcagggagggccaggaatggggccgccaatgccaactgtgaaccgtaaggcacaggaggcagccgcagcagtgatgcaggctgctgcgaactcagcacaaagcaggcaaggcagtttccccggcatgaaccagagtggacttatggcttccagctctccctacagccagcccatgaacaacagctctagcctgatgaacacgcaggcgccgccctacagcatggcgcccgccatggtgaacagctcggcagcatctgtgggtcttgcagatatgatgtctcctggtgaatccaaactgcccctgcctctcaaagcagacggcaaagaagaaggcactccacagcccgagagcaagtcaaaggatagctacagctctcagggtatttctcagcccccaaccccaggcaacctgccagtcccttccccaatgtcccccagctctgctagcatctcctcatttcatggagatgaaagtgatagcattagcagcccaggctggccaaagactccatcaagccctaagtccagctcctccaccactactggggagaagatcacgaaggtgtacgagctggggaatgagccagagagaaagctctgggtcgaccgatacctcaccttcatggaagagagaggctctcctgtctcaagtctgcctgccgtgggcaagaagcccctggacctgttccgactctacgtctgcgtcaaagagatcgggggtttggcccaggttaataaaaacaagaagtggcgtgagctggcaaccaacctaaacgttggcacctcaagcagtgcagcgagctccctgaaaaagcagtatattcagtacctgtttgcctttgagtgcaagatcgaacgtggggaggagcccccgccggaagtcttcagcaccggggacaccaaaaagcagcccaagctccagccgccatctcctgctaactcgggatccttgcaaggcccacagaccccccagtcaactggcagcaattccatggcagaggttccaggtgacctgaagccacctaccccagcctccacccctcacggccagatgactccaatgcaaggtggaagaagcagtacaatcagtgtgcacgacccattctcagatgtgagtgattcatccttcccgaaacggaactccatgactccaaacgccccctaccagcagggcatgagcatgcccgatgtgatgggcaggatgccctatgagcccaacaaggacccctttgggggaatgagaaaagtgcctggaagcagcgagccctttatgacgcaaggacagatgcccaacagcagcatgcaggacatgtacaaccaaagtccctccggagcaatgtctaacctgggcatggggcagcgccagcagtttccctatggagccagttacgaccgaaggcatgaaccttatgggcagcagtatccaggccaaggccctccctcgggacagccgccgtatggagggcaccagcccggcctgtacccacagcagccgaattacaaacgccatatggacggcatgtacgggcccccagccaagcgccacgagggcgacatgtacaacatgcagtacagcagccagcagcaggagatgtacaaccagtatggaggctcctactcgggcccggaccgcaggcccatccagggccagtacccgtatccctacagcagggagaggatgcagggcccggggcagatccagacacacggaatcccgcctcagatgatgggcggcccgctgcagtcgtcctccagtgaggggcctcagcagaatatgtgggcagcacgcaatgatatgccttatccctaccagaacaggcagggccctggcggccctacacaggcgcccccttacccaggcatgaaccgcacagacgatatgatggtacccgatcagaggataaatcatgagagccagtggccttctcacgtcagccagcgtcagccttatatgtcgtcctcagcctccatgcagcccatcacacgcccaccacagccgtcctaccagacgccaccgtcactgccaaatcacatctccagggcgcccagcccagcgtccttccagcgctccctggagaaccgcatgtctccaagcaagtctccttttctgccgtctatgaagatgcagaaggtcatgcccacggtccccacatcccaggtcaccgggccaccaccccaaccacccccaatcagaagggagatcacctttcctcctggctcagtagaagcatcacaaccagtcttgaaacaaaggcgaaagattacctccaaagatatcgttactcctgaggcgtggcgtgtgatgatgtcccttaaatcaggtcttttggctgagagtacgtgggctttggacactattaatattcttctgtatgatgacagcactgttgctactttcaatctctcccagttgtctggatttctcgaacttttagtcgagtactttagaaaatgcctgattgacatttttggaattcttatggaatatgaagtgggagaccccagccaaaaagcacttgatcacaacgcagcaaggaaggatgacagccagtccttggcagacgattctgggaaagaggaggaagatgctgaatgtattgatgacgacgaggaagacgaggaggatgaggaggaagacagcgagaagacagaaagcgatgaaaagagcagcatcgctctgactgccccggacgccgctgcagacccaaaggagaagcccaagcaagccagtaagttcgacaagctgccaataaagatagtcaaaaagaacaacctgtttgttgttgaccgatctgacaagttggggcgtgtgcaggagttcaatagtggccttctgcactggcagctcggcgggggtgacaccaccgagcacattcagactcactttgagagcaagatggaaattcctcctcgcaggcgcccacctccccccttaagctccgcaggtagaaagaaagagcaagaaggcaaaggcgactctgaagagcagcaagagaaaagcatcatagcaaccatcgatgacgtcctctctgctcggccaggggcattgcctgaagacgcaaaccctgggccccagaccgaaagcagtaagtttccctttggtatccagcaagccaaaagtcaccggaacatcaagctgctggaggacgagcccaggagccgagacgagactcctctgtgtaccatcgcgcactggcaggactcgctggctaagcgatgcatctgtgtgtccaatattgtccgtagcttgtcattcgtgcctggcaatgatgccgaaatgtccaaacatccaggcctggtgctgatcctggggaagctgattcttcttcaccacgagcatccagagagaaagcgagcaccgcagacctatgagaaagaggaggatgaggacaagggggtggcctgcagcaaagatgagtggtggtgggactgcctcgaggtcttgagggataacacgttggtcacgttggccaacatttccgggcagctagacttgtctgcttacacggaaagcatctgcttgccaattttggatggcttgctgcactggatggtgtgcccgtctgcagaggcacaagatccctttccaactgtgggacccaactcggtcctgtcgcctcagagacttgtgctggagaccctctgtaaactcagtatccaggacaataatgtggacctgatcttggccactcctccatttagtcgtcaggagaaattctatgctacattagttaggtacgttggggatcgcaaaaacccagtctgtcgagaaatgtccatggcgcttttatcgaaccttgcccaaggggacgcactagcagcaagggccatagctgtgcagaaaggaagcattggaaacttgataagcttcctagaggatggggtcacgatggcccagtaccagcagagccagcacaacctcatgcacatgcagcccccgcccctggaaccacctagcgtagacatgatgtgcagggcggccaaggctttgctagccatggccagagtggacgaaaaccgctcggaattccttttgcacgagggccggttgctggatatctcgatatcagctgtcctgaactctctggttgcatctgtcatctgtgatgtactgtttcagattgggcagttatgacataagtgagaaggcaagcatgtgtgagtgaagattagagggtcacatataactggctgttttctgttcttgtttatccagcgtaggaagaaggaaaagaaaatctttgctcctctgccccattcactatttaccaattgggaattaaagaaataattaatttgaacagttatgaaattaatatttgctgtctgtgtgtataagtacatcctttggggttttttttttctcttttttttaaccaaagttgctgtctagtgcattcaaaggtcactttttgttcttcacagatctttttaatgttctttcccatgttgtattgcatttttgggggaagcaaattgactttaaagaaaaaagttgtggcaaaagatgctaagatgcgaaaatttcaccacactgagtcaaaaaggtgaaaaattatccatttcctatgcgttttactcctcagagaatgaaaaaaactgcatcccatcacccaaagttctgtgcaatagaaatttctacagatacaggtataggggctcaaggaggtatgtcggtcagtagtcaaaactatgaaatgatactggtttctccacaggaatatggttccattaggctgggagcaaaaacaatgttttttaagattgagaatacatacctgacaacgatccggaaactgctcctcaccactcccgtcatgcctgctgtcggcgtttgaccttccacgtgacagttcttcacaattcctttcatcattttttaaatattttttttactgcctatgggctgtgatgtatatagaagttgtacattaaacataccctcatttttttcttttcttttttttttttttttttagtacaaagttttagtttctttttcatgatgtggtaactacgaagtgatggtagatttaaataattttttatttttattttatatattttttcattagggccatatctccaaaaaaagaaagaaaaaatacaaaaaacaaaaacaaaaaaaaaagagggtaatgtacaagtttctgtatgtataaagtcatgctcgatttcaggagagcagctgatcacaatttgcttcatgaatcaaggtgtggaaatggttatatatggattgatttagaaaatggttaccagtacagtcaaaaaagagaaaatgaaaaaaatacaactaaaaggaagaaacacaacttcaaagatttttcagtgatgagaatccacatttgtatttcaagataatgtagtttaaaaaaaaaaaaaagaaaaaaacttgatgtaaattcctccttttcctctggcttaatgaatatcatttattcagtataaaatctttatatgttccacatgttaagaataaatgtacattaaatcttgttaagcactgtgatgggtgttcttgaatactgttctagtttccttaaagtggtttcctagtaatcaagttatttacaagaaataggggaatgcagcagtgtattcacattataaaaccctacatttggaagagacctttaggggttacctactttagagtggggagcaacagtttgattttctcaaattacttagctaattagtctttctttgaagcaattaactctaacgacattgaggtatgatcattttcagtatttatgggaggtggctgctgacccacttgaggtgagatctcagaagcttaactggcctgaaaatgtaacattctgccttttactaactccatcttagtttaatcaaagttcaatctattccttgtttcttctgtgtgcctcagagttattttgcatttagtttactccaccgtgtataatatttatactgtgcaatgttaaaaaagaatctgttatattgtatgtggtgtacatagtgcaaagtgatgatttctatttcagggcatattatggttctcatattccttcctacctggtgcacagtagctttttaatactagtcacttctaatttaaactttctcttcctgggtcattgactgttactgtgtaataatcgatttctttgaaactgctgcataattatgctgttagtggacctctacctcttctcttccctctcccaatcacagtatactcagaatccccagcccctcgcatacattgtgtcggttcacattactcacagtaatatatggaagagttagacaagaacatgcagttacagtcattgtgagacgtgactctccagtgtcacgaggaaaaaaatcatcttttctgcaaacagtctctcatctgtcaactcccacattactgagtcaaacagtcttcttacataacaatgcaaccaaatatatgttgaattaaagacccatttataattctgctttaaatacatctgcttgctaagaacagatttcagtgctccaagcttcaaatatggagatttgtaagagggaattcaatattattctaatttctctcttacagagtacaaataaaaggtgtatacaaactccgaacatatccagtattccaattcctttgtcaatcagaagagtaaaataattaacaaaagactgttgttatggtttgcattgtaaccgatacgcagagtctgaccgttgggcaacaagtttttctatcctgatgcgcaacacagtctctagagactaatccaggaagactttagcctcctttccatattctcacccccgaatcaagatttacagaagcccacgaagaatttacagcctgcttgagatcatcttgcctataaactgagttattgctttgtcctaaaaattagtcggtttttttttttctatgaggcttttcagaaatttacaggatgcccagactttacatgtgtaccaaaaaaaaaaaaaagataaaaaataaaggtgcaaagaaagtttagtattttggaatggtgctataaagttgaa

SEQ ID NO: 17 - DNA, 10807 bp, Homo sapiens
gtcaaacaaataaccactgctgcagtggaaatgaactaaaatagtttttatggaaagctgaagaatgaaataattttggaaaactatactctaccaaaaccttggaagaagttgtgggaattacgccgatggcccgaaatcaaggcagaggcgaacgttctgaaaattacataactcctgggagagaccgtgcttttgcagattgcctaaatattttctttctccgtcggctccttgtaacctctgaggccaatagacaactttgtttcccagcccttctccctcttttgtctgctcgtcagactcgttttccacctttgtatcgttccttttagacgtgaaaacaaacatgcgaacgcccggttggcaaggagacagcggcccggggggaaagtgcaggttccggccgccccacgccgccgcgctccgagcggccgccgagccgccccggccccggccccggccccagccttagcccaagcccggctgggtcccgcgaggctgcgccggggcgggcggcggcggcccggggacgagtccagtccgcgttttgcgagtgcgcgggagtaatgcgagcgaagtggataatagttgctcgagctcgcccgctgcctctcaagccatgctgggcccgataggctcagctagtcgtgtatttacccatatccgggctagagaggaaaagagaaaagtttcatttaaacctgaactaaaaactttcaccatgaaagcacacagcaggagcaggcccagagcgtaaggcgtgcccggcccggcgctccggcggggcctgcggagggggagggggtcgcggcttcccggcgggccgcgtggatgcgcacaggaggggccgcggcctgaaaagtgggggttattgtctccccccgccccccgcccggcctcgccacgccgcggcgatcatggccgcgcgggcagcagcggcggcggcggcggcggcggcgcgggcgcgggcgcgggcaggcagcggcgaacggcgggcgccccccgggccgcggccggcgcccggagcccgggacctggaggcgggggcgcgcggcgcggcggcggcggcggcggcaccgggacccatgctggggggcggcggcgacggcggcggcggcctgaacagtgtgcaccaccaccccctgctcccccgtcacgaactcaacatggcccataacgcgggcgccgcggccgccgccggcacccacagcgccaagagcggcggctccgaggcggctctcaaggagggtggaagcgccgccgcgctgtcctcctcctcctcctcctccgcggcggcagcggcggcatcctcttcctcctcgtcgggcccgggctcggccatggagacggggctgctccccaaccacaaactgaaaaccgttggcgaagcccccgccgcgccgccccaccagcagcaccaccaccaccaccatgcccaccaccaccaccaccatgcccaccacctccaccaccaccacgcactacagcagcagctaaaccagttccagcagcagcagcagcagcagcaacagcagcagcagcagcagcagcaacagcaacatcccatttccaacaacaacagcttgggcggcgcgggcggcggcgcgcctcagcccggccccgacatggagcagccgcaacatggaggcgccaaggacagtgctgcgggcggccaggccgaccccccgggcccgccgctgctgagcaagccgggcgacgaggacgacgcgccgcccaagatgggggagccggcgggcggccgctacgagcacccgggcttgggcgccctgggcacgcagcagccgccggtcgccgtgcccgggggcggcggcggcccggcggccgtcccggagtttaataattactatggcagcgctgcccctgcgagcggcggccccggcggccgcgctgggccttgctttgatcaacatggcggacaacaaagccccgggatggggatgatgcactccgcctccgccgccgccgccggggcccccggcagcatggaccccctgcagaactcccacgaagggtaccccaacagccagtgcaaccattatccgggctacagccggcccggcgcgggcggcggcggcggcggcggcggcggaggaggaggaggcagcggaggaggaggaggaggaggaggagcaggagcaggaggagcaggagcgggagctgtggcggcggcggccgcggcggcggcggcagcagcaggaggcggcggcggcggcggctatgggggctcgtccgcggggtacggggtgctgagctccccccggcagcagggcggcggcatgatgatgggccccgggggcggcggggccgcgagcctcagcaaggcggccgccggctcggcggcggggggcttccagcgcttcgccggccagaaccagcacccgtcgggggccaccccgaccctcaatcagctgctcacctcgcccagccccatgatgcggagctacggcggcagctaccccgagtacagcagccccagcgcgccgccgccgccgccgtcgcagccccagtcccaggcggcggcggcgggggcggcggcgggcggccagcaggcggccgcgggcatgggcttgggcaaggacatgggcgcccagtacgccgctgccagcccggcctgggcggccgcgcaacaaaggagtcacccggcgatgagccccggcacccccggaccgaccatgggcagatcccagggcagcccaatggatccaatggtgatgaagagacctcagttgtatggcatgggcagtaaccctcattctcagcctcagcagagcagtccgtacccaggaggttcctatggccctccaggcccacagcggtatccaattggcatccagggtcggactcccggggccatggccggaatgcagtaccctcagcagcaggactctggagatgccacatggaaagaaacattctggttgatgccacctcagtatggacagcaaggtgtgagtggttactgccagcagggccaacagccatattacagccagcagccgcagcccccgcacctcccaccccaggcgcagtatctgccgtcccagtcccagcagaggtaccagccgcagcaggacatgtctcaggaaggctatggaactagatctcaacctcctctggcccccggaaaacctaaccatgaagacttgaacttaatacagcaagaaagaccatcaagtttaccagatctgtctggctccattgatgacctccccacgggaacggaagcaactttgagctcagcagtcagtgcatccgggtccacgagcagccaaggggatcagagcaacccggcgcagtcgcctttctccccacatgcgtcccctcatctctccagcatcccggggggcccatctccctctcctgttggctctcctgtaggaagcaaccagtctcgatctggcccaatctctcctgcaagtatcccaggtagtcagatgcctccgcagccacccgggagccagtcagaatccagttcccatcccgccttgagccagtcaccaatgccacaggaaagaggttttatggcaggcacacaaagaaaccctcagatggctcagtatggacctcaacagacaggaccatccatgtcgcctcatccttctcctgggggccagatgcatgctggaatcagtagctttcagcagagtaactcaagtgggacttacggtccacagatgagccagtatggaccacaaggtaactactccagacccccagcgtatagtggggtgcccagtgcaagctacagcggcccagggcccggtatgggtatcagtgccaacaaccagatgcatggacaagggccaagccagccatgtggtgctgtgcccctgggacgaatgccatcagctgggatgcagaacagaccatttcctggaaatatgagcagcatgacccccagttctcctggcatgtctcagcagggagggccaggaatggggccgccaatgccaactgtgaaccgtaaggcacaggaggcagccgcagcagtgatgcaggctgctgcgaactcagcacaaagcaggcaaggcagtttccccggcatgaaccagagtggacttatggcttccagctctccctacagccagcccatgaacaacagctctagcctgatgaacacgcaggcgccgccctacagcatggcgcccgccatggtgaacagctcggcagcatctgtgggtcttgcagatatgatgtctcctggtgaatccaaactgcccctgcctctcaaagcagacggcaaagaagaaggcactccacagcccgagagcaagtcaaagaagtccagctcctccaccactactggggagaagatcacgaaggtgtacgagctggggaatgagccagagagaaagctctgggtcgaccgatacctcaccttcatggaagagagaggctctcctgtctcaagtctgcctgccgtgggcaagaagcccctggacctgttccgactctacgtctgcgtcaaagagatcgggggtttggcccaggttaataaaaacaagaagtggcgtgagctggcaaccaacctaaacgttggcacctcaagcagtgcagcgagctccctgaaaaagcagtatattcagtacctgtttgcctttgagtgcaagatcgaacgtggggaggagcccccgccggaagtcttcagcaccggggacaccaaaaagcagcccaagctccagccgccatctcctgctaactcgggatccttgcaaggcccacagaccccccagtcaactggcagcaattccatggcagaggttccaggtgacctgaagccacctaccccagcctccacccctcacggccagatgactccaatgcaaggtggaagaagcagtacaatcagtgtgcacgacccattctcagatgtgagtgattcatccttcccgaaacggaactccatgactccaaacgccccctaccagcagggcatgagcatgcccgatgtgatgggcaggatgccctatgagcccaacaaggacccctttgggggaatgagaaaagtgcctggaagcagcgagccctttatgacgcaaggacagatgcccaacagcagcatgcaggacatgtacaaccaaagtccctccggagcaatgtctaacctgggcatggggcagcgccagcagtttccctatggagccagttacgaccgaaggcatgaaccttatgggcagcagtatccaggccaaggccctccctcgggacagccgccgtatggagggcaccagcccggcctgtacccacagcagccgaattacaaacgccatatggacggcatgtacgggcccccagccaagcgccacgagggcgacatgtacaacatgcagtacagcagccagcagcaggagatgtacaaccagtatggaggctcctactcgggcccggaccgcaggcccatccagggccagtacccgtatccctacagcagggagaggatgcagggcccggggcagatccagacacacggaatcccgcctcagatgatgggcggcccgctgcagtcgtcctccagtgaggggcctcagcagaatatgtgggcagcacgcaatgatatgccttatccctaccagaacaggcagggccctggcggccctacacaggcgcccccttacccaggcatgaaccgcacagacgatatgatggtacccgatcagaggataaatcatgagagccagtggccttctcacgtcagccagcgtcagccttatatgtcgtcctcagcctccatgcagcccatcacacgcccaccacagccgtcctaccagacgccaccgtcactgccaaatcacatctccagggcgcccagcccagcgtccttccagcgctccctggagaaccgcatgtctccaagcaagtctccttttctgccgtctatgaagatgcagaaggtcatgcccacggtccccacatcccaggtcaccgggccaccaccccaaccacccccaatcagaagggagatcacctttcctcctggctcagtagaagcatcacaaccagtcttgaaacaaaggcgaaagattacctccaaagatatcgttactcctgaggcgtggcgtgtgatgatgtcccttaaatcaggtcttttggctgagagtacgtgggctttggacactattaatattcttctgtatgatgacagcactgttgctactttcaatctctcccagttgtctggatttctcgaacttttagtcgagtactttagaaaatgcctgattgacatttttggaattcttatggaatatgaagtgggagaccccagccaaaaagcacttgatcacaacgcagcaaggaaggatgacagccagtccttggcagacgattctgggaaagaggaggaagatgctgaatgtattgatgacgacgaggaagacgaggaggatgaggaggaagacagcgagaagacagaaagcgatgaaaagagcagcatcgctctgactgccccggacgccgctgcagacccaaaggagaagcccaagcaagccagtaagttcgacaagctgccaataaagatagtcaaaaagaacaacctgtttgttgttgaccgatctgacaagttggggcgtgtgcaggagttcaatagtggccttctgcactggcagctcggcgggggtgacaccaccgagcacattcagactcactttgagagcaagatggaaattcctcctcgcaggcgcccacctccccccttaagctccgcaggtagaaagaaagagcaagaaggcaaaggcgactctgaagagcagcaagagaaaagcatcatagcaaccatcgatgacgtcctctctgctcggccaggggcattgcctgaagacgcaaaccctgggccccagaccgaaagcagtaagtttccctttggtatccagcaagccaaaagtcaccggaacatcaagctgctggaggacgagcccaggagccgagacgagactcctctgtgtaccatcgcgcactggcaggactcgctggctaagcgatgcatctgtgtgtccaatattgtccgtagcttgtcattcgtgcctggcaatgatgccgaaatgtccaaacatccaggcctggtgctgatcctggggaagctgattcttcttcaccacgagcatccagagagaaagcgagcaccgcagacctatgagaaagaggaggatgaggacaagggggtggcctgcagcaaagatgagtggtggtgggactgcctcgaggtcttgagggataacacgttggtcacgttggccaacatttccgggcagctagacttgtctgcttacacggaaagcatctgcttgccaattttggatggcttgctgcactggatggtgtgcccgtctgcagaggcacaagatccctttccaactgtgggacccaactcggtcctgtcgcctcagagacttgtgctggagaccctctgtaaactcagtatccaggacaataatgtggacctgatcttggccactcctccatttagtcgtcaggagaaattctatgctacattagttaggtacgttggggatcgcaaaaacccagtctgtcgagaaatgtccatggcgcttttatcgaaccttgcccaaggggacgcactagcagcaagggccatagctgtgcagaaaggaagcattggaaacttgataagcttcctagaggatggggtcacgatggcccagtaccagcagagccagcacaacctcatgcacatgcagcccccgcccctggaaccacctagcgtagacatgatgtgcagggcggccaaggctttgctagccatggccagagtggacgaaaaccgctcggaattccttttgcacgagggccggttgctggatatctcgatatcagctgtcctgaactctctggttgcatctgtcatctgtgatgtactgtttcagattgggcagttatgacataagtgagaaggcaagcatgtgtgagtgaagattagagggtcacatataactggctgttttctgttcttgtttatccagcgtaggaagaaggaaaagaaaatctttgctcctctgccccattcactatttaccaattgggaattaaagaaataattaatttgaacagttatgaaattaatatttgctgtctgtgtgtataagtacatcctttggggttttttttttctcttttttttaaccaaagttgctgtctagtgcattcaaaggtcactttttgttcttcacagatctttttaatgttctttcccatgttgtattgcatttttgggggaagcaaattgactttaaagaaaaaagttgtggcaaaagatgctaagatgcgaaaatttcaccacactgagtcaaaaaggtgaaaaattatccatttcctatgcgttttactcctcagagaatgaaaaaaactgcatcccatcacccaaagttctgtgcaatagaaatttctacagatacaggtataggggctcaaggaggtatgtcggtcagtagtcaaaactatgaaatgatactggtttctccacaggaatatggttccattaggctgggagcaaaaacaatgttttttaagattgagaatacatacctgacaacgatccggaaactgctcctcaccactcccgtcatgcctgctgtcggcgtttgaccttccacgtgacagttcttcacaattcctttcatcattttttaaatattttttttactgcctatgggctgtgatgtatatagaagttgtacattaaacataccctcatttttttcttttcttttttttttttttttttagtacaaagttttagtttctttttcatgatgtggtaactacgaagtgatggtagatttaaataattttttatttttattttatatattttttcattagggccatatctccaaaaaaagaaagaaaaaatacaaaaaacaaaaacaaaaaaaaaagagggtaatgtacaagtttctgtatgtataaagtcatgctcgatttcaggagagcagctgatcacaatttgcttcatgaatcaaggtgtggaaatggttatatatggattgatttagaaaatggttaccagtacagtcaaaaaagagaaaatgaaaaaaatacaactaaaaggaagaaacacaacttcaaagatttttcagtgatgagaatccacatttgtatttcaagataatgtagtttaaaaaaaaaaaaaagaaaaaaacttgatgtaaattcctccttttcctctggcttaatgaatatcatttattcagtataaaatctttatatgttccacatgttaagaataaatgtacattaaatcttgttaagcactgtgatgggtgttcttgaatactgttctagtttccttaaagtggtttcctagtaatcaagttatttacaagaaataggggaatgcagcagtgtattcacattataaaaccctacatttggaagagacctttaggggttacctactttagagtggggagcaacagtttgattttctcaaattacttagctaattagtctttctttgaagcaattaactctaacgacattgaggtatgatcattttcagtatttatgggaggtggctgctgacccacttgaggtgagatctcagaagcttaactggcctgaaaatgtaacattctgccttttactaactccatcttagtttaatcaaagttcaatctattccttgtttcttctgtgtgcctcagagttattttgcatttagtttactccaccgtgtataatatttatactgtgcaatgttaaaaaagaatctgttatattgtatgtggtgtacatagtgcaaagtgatgatttctatttcagggcatattatggttctcatattccttcctacctggtgcacagtagctttttaatactagtcacttctaatttaaactttctcttcctgggtcattgactgttactgtgtaataatcgatttctttgaaactgctgcataattatgctgttagtggacctctacctcttctcttccctctcccaatcacagtatactcagaatccccagcccctcgcatacattgtgtcggttcacattactcacagtaatatatggaagagttagacaagaacatgcagttacagtcattgtgagacgtgactctccagtgtcacgaggaaaaaaatcatcttttctgcaaacagtctctcatctgtcaactcccacattactgagtcaaacagtcttcttacataacaatgcaaccaaatatatgttgaattaaagacccatttataattctgctttaaatacatctgcttgctaagaacagatttcagtgctccaagcttcaaatatggagatttgtaagagggaattcaatattattctaatttctctcttacagagtacaaataaaaggtgtatacaaactccgaacatatccagtattccaattcctttgtcaatcagaagagtaaaataattaacaaaagactgttgttatggtttgcattgtaaccgatacgcagagtctgaccgttgggcaacaagtttttctatcctgatgcgcaacacagtctctagagactaatccaggaagactttagcctcctttccatattctcacccccgaatcaagatttacagaagcccacgaagaatttacagcctgcttgagatcatcttgcctataaactgagttattgctttgtcctaaaaattagtcggtttttttttttctatgaggcttttcagaaatttacaggatgcccagactttacatgtgtaccaaaaaaaaaaaaaagataaaaaataaaggtgcaaagaaagtttagtattttggaatggtgctataaagttgaa

SEQ ID NO: 18 - DNA, 10190 bp, Homo sapiens
aagccatgctgggcccgataggctcagctagtcgtgtatttacccatatccgggctagagaggaaaagagaaaagtttcatttaaacctgaactaaaaactttcaccatgaaagcacacagcaggagcaggcccagagcgtaaggcgtgcccggcccggcgctccggcggggcctgcggagggggagggggtcgcggcttcccggcgggccgcgtggatgcgcacaggaggggccgcggcctgaaaagtgggggttattgtctccccccgccccccgcccggcctcgccacgccgcggcgatcatggccgcgcgggcagcagcggcggcggcggcggcggcggcgcgggcgcgggcgcgggcaggcagcggcgaacggcgggcgccccccgggccgcggccggcgcccggagcccgggacctggaggcgggggcgcgcggcgcggcggcggcggcggcggcaccgggacccatgctggggggcggcggcgacggcggcggcggcctgaacagtgtgcaccaccaccccctgctcccccgtcacgaactcaacatggcccataacgcgggcgccgcggccgccgccggcacccacagcgccaagagcggcggctccgaggcggctctcaaggagggtggaagcgccgccgcgctgtcctcctcctcctcctcctccgcggcggcagcggcggcatcctcttcctcctcgtcgggcccgggctcggccatggagacggggctgctccccaaccacaaactgaaaaccgttggcgaagcccccgccgcgccgccccaccagcagcaccaccaccaccaccatgcccaccaccaccaccaccatgcccaccacctccaccaccaccacgcactacagcagcagctaaaccagttccagcagcagcagcagcagcagcaacagcagcagcagcagcagcagcaacagcaacatcccatttccaacaacaacagcttgggcggcgcgggcggcggcgcgcctcagcccggccccgacatggagcagccgcaacatggaggcgccaaggacagtgctgcgggcggccaggccgaccccccgggcccgccgctgctgagcaagccgggcgacgaggacgacgcgccgcccaagatgggggagccggcgggcggccgctacgagcacccgggcttgggcgccctgggcacgcagcagccgccggtcgccgtgcccgggggcggcggcggcccggcggccgtcccggagtttaataattactatggcagcgctgcccctgcgagcggcggccccggcggccgcgctgggccttgctttgatcaacatggcggacaacaaagccccgggatggggatgatgcactccgcctccgccgccgccgccggggcccccggcagcatggaccccctgcagaactcccacgaagggtaccccaacagccagtgcaaccattatccgggctacagccggcccggcgcgggcggcggcggcggcggcggcggcggaggaggaggaggcagcggaggaggaggaggaggaggaggagcaggagcaggaggagcaggagcgggagctgtggcggcggcggccgcggcggcggcggcagcagcaggaggcggcggcggcggcggctatgggggctcgtccgcggggtacggggtgctgagctccccccggcagcagggcggcggcatgatgatgggccccgggggcggcggggccgcgagcctcagcaaggcggccgccggctcggcggcggggggcttccagcgcttcgccggccagaaccagcacccgtcgggggccaccccgaccctcaatcagctgctcacctcgcccagccccatgatgcggagctacggcggcagctaccccgagtacagcagccccagcgcgccgccgccgccgccgtcgcagccccagtcccaggcggcggcggcgggggcggcggcgggcggccagcaggcggccgcgggcatgggcttgggcaaggacatgggcgcccagtacgccgctgccagcccggcctgggcggccgcgcaacaaaggagtcacccggcgatgagccccggcacccccggaccgaccatgggcagatcccagggcagcccaatggatccaatggtgatgaagagacctcagttgtatggcatgggcagtaaccctcattctcagcctcagcagagcagtccgtacccaggaggttcctatggccctccaggcccacagcggtatccaattggcatccagggtcggactcccggggccatggccggaatgcagtaccctcagcagcaggactctggagatgccacatggaaagaaacattctggttgatgccacctcagtatggacagcaaggtgtgagtggttactgccagcagggccaacagccatattacagccagcagccgcagcccccgcacctcccaccccaggcgcagtatctgccgtcccagtcccagcagaggtaccagccgcagcaggacatgtctcaggaaggctatggaactagatctcaacctcctctggcccccggaaaacctaaccatgaagacttgaacttaatacagcaagaaagaccatcaagtttaccagatctgtctggctccattgatgacctccccacgggaacggaagcaactttgagctcagcagtcagtgcatccgggtccacgagcagccaaggggatcagagcaacccggcgcagtcgcctttctccccacatgcgtcccctcatctctccagcatcccggggggcccatctccctctcctgttggctctcctgtaggaagcaaccagtctcgatctggcccaatctctcctgcaagtatcccaggtagtcagatgcctccgcagccacccgggagccagtcagaatccagttcccatcccgccttgagccagtcaccaatgccacaggaaagaggttttatggcaggcacacaaagaaaccctcagatggctcagtatggacctcaacagacaggaccatccatgtcgcctcatccttctcctgggggccagatgcatgctggaatcagtagctttcagcagagtaactcaagtgggacttacggtccacagatgagccagtatggaccacaaggtaactactccagacccccagcgtatagtggggtgcccagtgcaagctacagcggcccagggcccggtatgggtatcagtgccaacaaccagatgcatggacaagggccaagccagccatgtggtgctgtgcccctgggacgaatgccatcagctgggatgcagaacagaccatttcctggaaatatgagcagcatgacccccagttctcctggcatgtctcagcagggagggccaggaatggggccgccaatgccaactgtgaaccgtaaggcacaggaggcagccgcagcagtgatgcaggctgctgcgaactcagcacaaagcaggcaaggcagtttccccggcatgaaccagagtggacttatggcttccagctctccctacagccagcccatgaacaacagctctagcctgatgaacacgcaggcgccgccctacagcatggcgcccgccatggtgaacagctcggcagcatctgtgggtcttgcagatatgatgtctcctggtgaatccaaactgcccctgcctctcaaagcagacggcaaagaagaaggcactccacagcccgagagcaagtcaaagaagtccagctcctccaccactactggggagaagatcacgaaggtgtacgagctggggaatgagccagagagaaagctctgggtcgaccgatacctcaccttcatggaagagagaggctctcctgtctcaagtctgcctgccgtgggcaagaagcccctggacctgttccgactctacgtctgcgtcaaagagatcgggggtttggcccaggttaataaaaacaagaagtggcgtgagctggcaaccaacctaaacgttggcacctcaagcagtgcagcgagctccctgaaaaagcagtatattcagtacctgtttgcctttgagtgcaagatcgaacgtggggaggagcccccgccggaagtcttcagcaccggggacaccaaaaagcagcccaagctccagccgccatctcctgctaactcgggatccttgcaaggcccacagaccccccagtcaactggcagcaattccatggcagaggttccaggtgacctgaagccacctaccccagcctccacccctcacggccagatgactccaatgcaaggtggaagaagcagtacaatcagtgtgcacgacccattctcagatgtgagtgattcatccttcccgaaacggaactccatgactccaaacgccccctaccagcagggcatgagcatgcccgatgtgatgggcaggatgccctatgagcccaacaaggacccctttgggggaatgagaaaagtgcctggaagcagcgagccctttatgacgcaaggacagatgcccaacagcagcatgcaggacatgtacaaccaaagtccctccggagcaatgtctaacctgggcatggggcagcgccagcagtttccctatggagccagttacgaccgaaggcatgaaccttatgggcagcagtatccaggccaaggccctccctcgggacagccgccgtatggagggcaccagcccggcctgtacccacagcagccgaattacaaacgccatatggacggcatgtacgggcccccagccaagcgccacgagggcgacatgtacaacatgcagtacagcagccagcagcaggagatgtacaaccagtatggaggctcctactcgggcccggaccgcaggcccatccagggccagtacccgtatccctacagcagggagaggatgcagggcccggggcagatccagacacacggaatcccgcctcagatgatgggcggcccgctgcagtcgtcctccagtgaggggcctcagcagaatatgtgggcagcacgcaatgatatgccttatccctaccagaacaggcagggccctggcggccctacacaggcgcccccttacccaggcatgaaccgcacagacgatatgatggtacccgatcagaggataaatcatgagagccagtggccttctcacgtcagccagcgtcagccttatatgtcgtcctcagcctccatgcagcccatcacacgcccaccacagccgtcctaccagacgccaccgtcactgccaaatcacatctccagggcgcccagcccagcgtccttccagcgctccctggagaaccgcatgtctccaagcaagtctccttttctgccgtctatgaagatgcagaaggtcatgcccacggtccccacatcccaggtcaccgggccaccaccccaaccacccccaatcagaagggagatcacctttcctcctggctcagtagaagcatcacaaccagtcttgaaacaaaggcgaaagattacctccaaagatatcgttactcctgaggcgtggcgtgtgatgatgtcccttaaatcaggtcttttggctgagagtacgtgggctttggacactattaatattcttctgtatgatgacagcactgttgctactttcaatctctcccagttgtctggatttctcgaacttttagtcgagtactttagaaaatgcctgattgacatttttggaattcttatggaatatgaagtgggagaccccagccaaaaagcacttgatcacaacgcagcaaggaaggatgacagccagtccttggcagacgattctgggaaagaggaggaagatgctgaatgtattgatgacgacgaggaagacgaggaggatgaggaggaagacagcgagaagacagaaagcgatgaaaagagcagcatcgctctgactgccccggacgccgctgcagacccaaaggagaagcccaagcaagccagtaagttcgacaagctgccaataaagatagtcaaaaagaacaacctgtttgttgttgaccgatctgacaagttggggcgtgtgcaggagttcaatagtggccttctgcactggcagctcggcgggggtgacaccaccgagcacattcagactcactttgagagcaagatggaaattcctcctcgcaggcgcccacctccccccttaagctccgcaggtagaaagaaagagcaagaaggcaaaggcgactctgaagagcagcaagagaaaagcatcatagcaaccatcgatgacgtcctctctgctcggccaggggcattgcctgaagacgcaaaccctgggccccagaccgaaagcagtaagtttccctttggtatccagcaagccaaaagtcaccggaacatcaagctgctggaggacgagcccaggagccgagacgagactcctctgtgtaccatcgcgcactggcaggactcgctggctaagcgatgcatctgtgtgtccaatattgtccgtagcttgtcattcgtgcctggcaatgatgccgaaatgtccaaacatccaggcctggtgctgatcctggggaagctgattcttcttcaccacgagcatccagagagaaagcgagcaccgcagacctatgagaaagaggaggatgaggacaagggggtggcctgcagcaaagatgagtggtggtgggactgcctcgaggtcttgagggataacacgttggtcacgttggccaacatttccgggcagctagacttgtctgcttacacggaaagcatctgcttgccaattttggatggcttgctgcactggatggtgtgcccgtctgcagaggcacaagatccctttccaactgtgggacccaactcggtcctgtcgcctcagagacttgtgctggagaccctctgtaaactcagtatccaggacaataatgtggacctgatcttggccactcctccatttagtcgtcaggagaaattctatgctacattagttaggtacgttggggatcgcaaaaacccagtctgtcgagaaatgtccatggcgcttttatcgaaccttgcccaaggggacgcactagcagcaagggccatagctgtgcagaaaggaagcattggaaacttgataagcttcctagaggatggggtcacgatggcccagtaccagcagagccagcacaacctcatgcacatgcagcccccgcccctggaaccacctagcgtagacatgatgtgcagggcggccaaggctttgctagccatggccagagtggacgaaaaccgctcggaattccttttgcacgagggccggttgctggatatctcgatatcagctgtcctgaactctctggttgcatctgtcatctgtgatgtactgtttcagattgggcagttatgacataagtgagaaggcaagcatgtgtgagtgaagattagagggtcacatataactggctgttttctgttcttgtttatccagcgtaggaagaaggaaaagaaaatctttgctcctctgccccattcactatttaccaattgggaattaaagaaataattaatttgaacagttatgaaattaatatttgctgtctgtgtgtataagtacatcctttggggttttttttttctcttttttttaaccaaagttgctgtctagtgcattcaaaggtcactttttgttcttcacagatctttttaatgttctttcccatgttgtattgcatttttgggggaagcaaattgactttaaagaaaaaagttgtggcaaaagatgctaagatgcgaaaatttcaccacactgagtcaaaaaggtgaaaaattatccatttcctatgcgttttactcctcagagaatgaaaaaaactgcatcccatcacccaaagttctgtgcaatagaaatttctacagatacaggtataggggctcaaggaggtatgtcggtcagtagtcaaaactatgaaatgatactggtttctccacaggaatatggttccattaggctgggagcaaaaacaatgttttttaagattgagaatacatacctgacaacgatccggaaactgctcctcaccactcccgtcatgcctgctgtcggcgtttgaccttccacgtgacagttcttcacaattcctttcatcattttttaaatattttttttactgcctatgggctgtgatgtatatagaagttgtacattaaacataccctcatttttttcttttcttttttttttttttttttagtacaaagttttagtttctttttcatgatgtggtaactacgaagtgatggtagatttaaataattttttatttttattttatatattttttcattagggccatatctccaaaaaaagaaagaaaaaatacaaaaaacaaaaacaaaaaaaaaagagggtaatgtacaagtttctgtatgtataaagtcatgctcgatttcaggagagcagctgatcacaatttgcttcatgaatcaaggtgtggaaatggttatatatggattgatttagaaaatggttaccagtacagtcaaaaaagagaaaatgaaaaaaatacaactaaaaggaagaaacacaacttcaaagatttttcagtgatgagaatccacatttgtatttcaagataatgtagtttaaaaaaaaaaaaaagaaaaaaacttgatgtaaattcctccttttcctctggcttaatgaatatcatttattcagtataaaatctttatatgttccacatgttaagaataaatgtacattaaatcttgttaagcactgtgatgggtgttcttgaatactgttctagtttccttaaagtggtttcctagtaatcaagttatttacaagaaataggggaatgcagcagtgtattcacattataaaaccctacatttggaagagacctttaggggttacctactttagagtggggagcaacagtttgattttctcaaattacttagctaattagtctttctttgaagcaattaactctaacgacattgaggtatgatcattttcagtatttatgggaggtggctgctgacccacttgaggtgagatctcagaagcttaactggcctgaaaatgtaacattctgccttttactaactccatcttagtttaatcaaagttcaatctattccttgtttcttctgtgtgcctcagagttattttgcatttagtttactccaccgtgtataatatttatactgtgcaatgttaaaaaagaatctgttatattgtatgtggtgtacatagtgcaaagtgatgatttctatttcagggcatattatggttctcatattccttcctacctggtgcacagtagctttttaatactagtcacttctaatttaaactttctcttcctgggtcattgactgttactgtgtaataatcgatttctttgaaactgctgcataattatgctgttagtggacctctacctcttctcttccctctcccaatcacagtatactcagaatccccagcccctcgcatacattgtgtcggttcacattactcacagtaatatatggaagagttagacaagaacatgcagttacagtcattgtgagacgtgactctccagtgtcacgaggaaaaaaatcatcttttctgcaaacagtctctcatctgtcaactcccacattactgagtcaaacagtcttcttacataacaatgcaaccaaatatatgttgaattaaagacccatttataattctgctttaaatacatctgcttgctaagaacagatttcagtgctccaagcttcaaatatggagatttgtaagagggaattcaatattattctaatttctctcttacagagtacaaataaaaggtgtatacaaactccgaacatatccagtattccaattcctttgtcaatcagaagagtaaaataattaacaaaagactgttgttatggtttgcattgtaaccgatacgcagagtctgaccgttgggcaacaagtttttctatcctgatgcgcaacacagtctctagagactaatccaggaagactttagcctcctttccatattctcacccccgaatcaagatttacagaagcccacgaagaatttacagcctgcttgagatcatcttgcctataaactgagttattgctttgtcctaaaaattagtcggtttttttttttctatgaggcttttcagaaatttacaggatgcccagactttacatgtgtaccaaaaaaaaaaaaaagataaaaaataaaggtgcaaagaaagtttagtattttggaatggtgctataaagttgaa

SEQ ID NO: 19 - DNA, 10310 bp, Homo sapiens
aagccatgctgggcccgataggctcagctagtcgtgtatttacccatatccgggctagagaggaaaagagaaaagtttcatttaaacctgaactaaaaactttcaccatgaaagcacacagcaggagcaggcccagagcgtaaggcgtgcccggcccggcgctccggcggggcctgcggagggggagggggtcgcggcttcccggcgggccgcgtggatgcgcacaggaggggccgcggcctgaaaagtgggggttattgtctccccccgccccccgcccggcctcgccacgccgcggcgatcatggccgcgcgggcagcagcggcggcggcggcggcggcggcgcgggcgcgggcgcgggcaggcagcggcgaacggcgggcgccccccgggccgcggccggcgcccggagcccgggacctggaggcgggggcgcgcggcgcggcggcggcggcggcggcaccgggacccatgctggggggcggcggcgacggcggcggcggcctgaacagtgtgcaccaccaccccctgctcccccgtcacgaactcaacatggcccataacgcgggcgccgcggccgccgccggcacccacagcgccaagagcggcggctccgaggcggctctcaaggagggtggaagcgccgccgcgctgtcctcctcctcctcctcctccgcggcggcagcggcggcatcctcttcctcctcgtcgggcccgggctcggccatggagacggggctgctccccaaccacaaactgaaaaccgttggcgaagcccccgccgcgccgccccaccagcagcaccaccaccaccaccatgcccaccaccaccaccaccatgcccaccacctccaccaccaccacgcactacagcagcagctaaaccagttccagcagcagcagcagcagcagcaacagcagcagcagcagcagcagcaacagcaacatcccatttccaacaacaacagcttgggcggcgcgggcggcggcgcgcctcagcccggccccgacatggagcagccgcaacatggaggcgccaaggacagtgctgcgggcggccaggccgaccccccgggcccgccgctgctgagcaagccgggcgacgaggacgacgcgccgcccaagatgggggagccggcgggcggccgctacgagcacccgggcttgggcgccctgggcacgcagcagccgccggtcgccgtgcccgggggcggcggcggcccggcggccgtcccggagtttaataattactatggcagcgctgcccctgcgagcggcggccccggcggccgcgctgggccttgctttgatcaacatggcggacaacaaagccccgggatggggatgatgcactccgcctccgccgccgccgccggggcccccggcagcatggaccccctgcagaactcccacgaagggtaccccaacagccagtgcaaccattatccgggctacagccggcccggcgcgggcggcggcggcggcggcggcggcggaggaggaggaggcagcggaggaggaggaggaggaggaggagcaggagcaggaggagcaggagcgggagctgtggcggcggcggccgcggcggcggcggcagcagcaggaggcggcggcggcggcggctatgggggctcgtccgcggggtacggggtgctgagctccccccggcagcagggcggcggcatgatgatgggccccgggggcggcggggccgcgagcctcagcaaggcggccgccggctcggcggcggggggcttccagcgcttcgccggccagaaccagcacccgtcgggggccaccccgaccctcaatcagctgctcacctcgcccagccccatgatgcggagctacggcggcagctaccccgagtacagcagccccagcgcgccgccgccgccgccgtcgcagccccagtcccaggcggcggcggcgggggcggcggcgggcggccagcaggcggccgcgggcatgggcttgggcaaggacatgggcgcccagtacgccgctgccagcccggcctgggcggccgcgcaacaaaggagtcacccggcgatgagccccggcacccccggaccgaccatgggcagatcccagggcagcccaatggatccaatggtgatgaagagacctcagttgtatggcatgggcagtaaccctcattctcagcctcagcagagcagtccgtacccaggaggttcctatggccctccaggcccacagcggtatccaattggcatccagggtcggactcccggggccatggccggaatgcagtaccctcagcagcagatgccacctcagtatggacagcaaggtgtgagtggttactgccagcagggccaacagccatattacagccagcagccgcagcccccgcacctcccaccccaggcgcagtatctgccgtcccagtcccagcagaggtaccagccgcagcaggacatgtctcaggaaggctatggaactagatctcaacctcctctggcccccggaaaacctaaccatgaagacttgaacttaatacagcaagaaagaccatcaagtttaccagatctgtctggctccattgatgacctccccacgggaacggaagcaactttgagctcagcagtcagtgcatccgggtccacgagcagccaaggggatcagagcaacccggcgcagtcgcctttctccccacatgcgtcccctcatctctccagcatcccggggggcccatctccctctcctgttggctctcctgtaggaagcaaccagtctcgatctggcccaatctctcctgcaagtatcccaggtagtcagatgcctccgcagccacccgggagccagtcagaatccagttcccatcccgccttgagccagtcaccaatgccacaggaaagaggttttatggcaggcacacaaagaaaccctcagatggctcagtatggacctcaacagacaggaccatccatgtcgcctcatccttctcctgggggccagatgcatgctggaatcagtagctttcagcagagtaactcaagtgggacttacggtccacagatgagccagtatggaccacaaggtaactactccagacccccagcgtatagtggggtgcccagtgcaagctacagcggcccagggcccggtatgggtatcagtgccaacaaccagatgcatggacaagggccaagccagccatgtggtgctgtgcccctgggacgaatgccatcagctgggatgcagaacagaccatttcctggaaatatgagcagcatgacccccagttctcctggcatgtctcagcagggagggccaggaatggggccgccaatgccaactgtgaaccgtaaggcacaggaggcagccgcagcagtgatgcaggctgctgcgaactcagcacaaagcaggcaaggcagtttccccggcatgaaccagagtggacttatggcttccagctctccctacagccagcccatgaacaacagctctagcctgatgaacacgcaggcgccgccctacagcatggcgcccgccatggtgaacagctcggcagcatctgtgggtcttgcagatatgatgtctcctggtgaatccaaactgcccctgcctctcaaagcagacggcaaagaagaaggcactccacagcccgagagcaagtcaaaggatagctacagctctcagggtatttctcagcccccaaccccaggcaacctgccagtcccttccccaatgtcccccagctctgctagcatctcctcatttcatggagatgaaagtgatagcattagcagcccaggctggccaaagactccatcaagccctaagtccagctcctccaccactactggggagaagatcacgaaggtgtacgagctggggaatgagccagagagaaagctctgggtcgaccgatacctcaccttcatggaagagagaggctctcctgtctcaagtctgcctgccgtgggcaagaagcccctggacctgttccgactctacgtctgcgtcaaagagatcgggggtttggcccaggttaataaaaacaagaagtggcgtgagctggcaaccaacctaaacgttggcacctcaagcagtgcagcgagctccctgaaaaagcagtatattcagtacctgtttgcctttgagtgcaagatcgaacgtggggaggagcccccgccggaagtcttcagcaccggggacaccaaaaagcagcccaagctccagccgccatctcctgctaactcgggatccttgcaaggcccacagaccccccagtcaactggcagcaattccatggcagaggttccaggtgacctgaagccacctaccccagcctccacccctcacggccagatgactccaatgcaaggtggaagaagcagtacaatcagtgtgcacgacccattctcagatgtgagtgattcatccttcccgaaacggaactccatgactccaaacgccccctaccagcagggcatgagcatgcccgatgtgatgggcaggatgccctatgagcccaacaaggacccctttgggggaatgagaaaagtgcctggaagcagcgagccctttatgacgcaaggacagatgcccaacagcagcatgcaggacatgtacaaccaaagtccctccggagcaatgtctaacctgggcatggggcagcgccagcagtttccctatggagccagttacgaccgaaggcatgaaccttatgggcagcagtatccaggccaaggccctccctcgggacagccgccgtatggagggcaccagcccggcctgtacccacagcagccgaattacaaacgccatatggacggcatgtacgggcccccagccaagcgccacgagggcgacatgtacaacatgcagtacagcagccagcagcaggagatgtacaaccagtatggaggctcctactcgggcccggaccgcaggcccatccagggccagtacccgtatccctacagcagggagaggatgcagggcccggggcagatccagacacacggaatcccgcctcagatgatgggcggcccgctgcagtcgtcctccagtgaggggcctcagcagaatatgtgggcagcacgcaatgatatgccttatccctaccagaacaggcagggccctggcggccctacacaggcgcccccttacccaggcatgaaccgcacagacgatatgatggtacccgatcagaggataaatcatgagagccagtggccttctcacgtcagccagcgtcagccttatatgtcgtcctcagcctccatgcagcccatcacacgcccaccacagccgtcctaccagacgccaccgtcactgccaaatcacatctccagggcgcccagcccagcgtccttccagcgctccctggagaaccgcatgtctccaagcaagtctccttttctgccgtctatgaagatgcagaaggtcatgcccacggtccccacatcccaggtcaccgggccaccaccccaaccacccccaatcagaagggagatcacctttcctcctggctcagtagaagcatcacaaccagtcttgaaacaaaggcgaaagattacctccaaagatatcgttactcctgaggcgtggcgtgtgatgatgtcccttaaatcaggtcttttggctgagagtacgtgggctttggacactattaatattcttctgtatgatgacagcactgttgctactttcaatctctcccagttgtctggatttctcgaacttttagtcgagtactttagaaaatgcctgattgacatttttggaattcttatggaatatgaagtgggagaccccagccaaaaagcacttgatcacaacgcagcaaggaaggatgacagccagtccttggcagacgattctgggaaagaggaggaagatgctgaatgtattgatgacgacgaggaagacgaggaggatgaggaggaagacagcgagaagacagaaagcgatgaaaagagcagcatcgctctgactgccccggacgccgctgcagacccaaaggagaagcccaagcaagccagtaagttcgacaagctgccaataaagatagtcaaaaagaacaacctgtttgttgttgaccgatctgacaagttggggcgtgtgcaggagttcaatagtggccttctgcactggcagctcggcgggggtgacaccaccgagcacattcagactcactttgagagcaagatggaaattcctcctcgcaggcgcccacctccccccttaagctccgcaggtagaaagaaagagcaagaaggcaaaggcgactctgaagagcagcaagagaaaagcatcatagcaaccatcgatgacgtcctctctgctcggccaggggcattgcctgaagacgcaaaccctgggccccagaccgaaagcagtaagtttccctttggtatccagcaagccaaaagtcaccggaacatcaagctgctggaggacgagcccaggagccgagacgagactcctctgtgtaccatcgcgcactggcaggactcgctggctaagcgatgcatctgtgtgtccaatattgtccgtagcttgtcattcgtgcctggcaatgatgccgaaatgtccaaacatccaggcctggtgctgatcctggggaagctgattcttcttcaccacgagcatccagagagaaagcgagcaccgcagacctatgagaaagaggaggatgaggacaagggggtggcctgcagcaaagatgagtggtggtgggactgcctcgaggtcttgagggataacacgttggtcacgttggccaacatttccgggcagctagacttgtctgcttacacggaaagcatctgcttgccaattttggatggcttgctgcactggatggtgtgcccgtctgcagaggcacaagatccctttccaactgtgggacccaactcggtcctgtcgcctcagagacttgtgctggagaccctctgtaaactcagtatccaggacaataatgtggacctgatcttggccactcctccatttagtcgtcaggagaaattctatgctacattagttaggtacgttggggatcgcaaaaacccagtctgtcgagaaatgtccatggcgcttttatcgaaccttgcccaaggggacgcactagcagcaagggccatagctgtgcagaaaggaagcattggaaacttgataagcttcctagaggatggggtcacgatggcccagtaccagcagagccagcacaacctcatgcacatgcagcccccgcccctggaaccacctagcgtagacatgatgtgcagggcggccaaggctttgctagccatggccagagtggacgaaaaccgctcggaattccttttgcacgagggccggttgctggatatctcgatatcagctgtcctgaactctctggttgcatctgtcatctgtgatgtactgtttcagattgggcagttatgacataagtgagaaggcaagcatgtgtgagtgaagattagagggtcacatataactggctgttttctgttcttgtttatccagcgtaggaagaaggaaaagaaaatctttgctcctctgccccattcactatttaccaattgggaattaaagaaataattaatttgaacagttatgaaattaatatttgctgtctgtgtgtataagtacatcctttggggttttttttttctcttttttttaaccaaagttgctgtctagtgcattcaaaggtcactttttgttcttcacagatctttttaatgttctttcccatgttgtattgcatttttgggggaagcaaattgactttaaagaaaaaagttgtggcaaaagatgctaagatgcgaaaatttcaccacactgagtcaaaaaggtgaaaaattatccatttcctatgcgttttactcctcagagaatgaaaaaaactgcatcccatcacccaaagttctgtgcaatagaaatttctacagatacaggtataggggctcaaggaggtatgtcggtcagtagtcaaaactatgaaatgatactggtttctccacaggaatatggttccattaggctgggagcaaaaacaatgttttttaagattgagaatacatacctgacaacgatccggaaactgctcctcaccactcccgtcatgcctgctgtcggcgtttgaccttccacgtgacagttcttcacaattcctttcatcattttttaaatattttttttactgcctatgggctgtgatgtatatagaagttgtacattaaacataccctcatttttttcttttcttttttttttttttttttagtacaaagttttagtttctttttcatgatgtggtaactacgaagtgatggtagatttaaataattttttatttttattttatatattttttcattagggccatatctccaaaaaaagaaagaaaaaatacaaaaaacaaaaacaaaaaaaaaagagggtaatgtacaagtttctgtatgtataaagtcatgctcgatttcaggagagcagctgatcacaatttgcttcatgaatcaaggtgtggaaatggttatatatggattgatttagaaaatggttaccagtacagtcaaaaaagagaaaatgaaaaaaatacaactaaaaggaagaaacacaacttcaaagatttttcagtgatgagaatccacatttgtatttcaagataatgtagtttaaaaaaaaaaaaaagaaaaaaacttgatgtaaattcctccttttcctctggcttaatgaatatcatttattcagtataaaatctttatatgttccacatgttaagaataaatgtacattaaatcttgttaagcactgtgatgggtgttcttgaatactgttctagtttccttaaagtggtttcctagtaatcaagttatttacaagaaataggggaatgcagcagtgtattcacattataaaaccctacatttggaagagacctttaggggttacctactttagagtggggagcaacagtttgattttctcaaattacttagctaattagtctttctttgaagcaattaactctaacgacattgaggtatgatcattttcagtatttatgggaggtggctgctgacccacttgaggtgagatctcagaagcttaactggcctgaaaatgtaacattctgccttttactaactccatcttagtttaatcaaagttcaatctattccttgtttcttctgtgtgcctcagagttattttgcatttagtttactccaccgtgtataatatttatactgtgcaatgttaaaaaagaatctgttatattgtatgtggtgtacatagtgcaaagtgatgatttctatttcagggcatattatggttctcatattccttcctacctggtgcacagtagctttttaatactagtcacttctaatttaaactttctcttcctgggtcattgactgttactgtgtaataatcgatttctttgaaactgctgcataattatgctgttagtggacctctacctcttctcttccctctcccaatcacagtatactcagaatccccagcccctcgcatacattgtgtcggttcacattactcacagtaatatatggaagagttagacaagaacatgcagttacagtcattgtgagacgtgactctccagtgtcacgaggaaaaaaatcatcttttctgcaaacagtctctcatctgtcaactcccacattactgagtcaaacagtcttcttacataacaatgcaaccaaatatatgttgaattaaagacccatttataattctgctttaaatacatctgcttgctaagaacagatttcagtgctccaagcttcaaatatggagatttgtaagagggaattcaatattattctaatttctctcttacagagtacaaataaaaggtgtatacaaactccgaacatatccagtattccaattcctttgtcaatcagaagagtaaaataattaacaaaagactgttgttatggtttgcattgtaaccgatacgcagagtctgaccgttgggcaacaagtttttctatcctgatgcgcaacacagtctctagagactaatccaggaagactttagcctcctttccatattctcacccccgaatcaagatttacagaagcccacgaagaatttacagcctgcttgagatcatcttgcctataaactgagttattgctttgtcctaaaaattagtcggtttttttttttctatgaggcttttcagaaatttacaggatgcccagactttacatgtgtaccaaaaaaaaaaaaaagataaaaaataaaggtgcaaagaaagtttagtattttggaatggtgctataaagttgaa

SEQ ID NO: 20 - DNA, 10151 bp, Homo sapiens
aagccatgctgggcccgataggctcagctagtcgtgtatttacccatatccgggctagagaggaaaagagaaaagtttcatttaaacctgaactaaaaactttcaccatgaaagcacacagcaggagcaggcccagagcgtaaggcgtgcccggcccggcgctccggcggggcctgcggagggggagggggtcgcggcttcccggcgggccgcgtggatgcgcacaggaggggccgcggcctgaaaagtgggggttattgtctccccccgccccccgcccggcctcgccacgccgcggcgatcatggccgcgcgggcagcagcggcggcggcggcggcggcggcgcgggcgcgggcgcgggcaggcagcggcgaacggcgggcgccccccgggccgcggccggcgcccggagcccgggacctggaggcgggggcgcgcggcgcggcggcggcggcggcggcaccgggacccatgctggggggcggcggcgacggcggcggcggcctgaacagtgtgcaccaccaccccctgctcccccgtcacgaactcaacatggcccataacgcgggcgccgcggccgccgccggcacccacagcgccaagagcggcggctccgaggcggctctcaaggagggtggaagcgccgccgcgctgtcctcctcctcctcctcctccgcggcggcagcggcggcatcctcttcctcctcgtcgggcccgggctcggccatggagacggggctgctccccaaccacaaactgaaaaccgttggcgaagcccccgccgcgccgccccaccagcagcaccaccaccaccaccatgcccaccaccaccaccaccatgcccaccacctccaccaccaccacgcactacagcagcagctaaaccagttccagcagcagcagcagcagcagcaacagcagcagcagcagcagcagcaacagcaacatcccatttccaacaacaacagcttgggcggcgcgggcggcggcgcgcctcagcccggccccgacatggagcagccgcaacatggaggcgccaaggacagtgctgcgggcggccaggccgaccccccgggcccgccgctgctgagcaagccgggcgacgaggacgacgcgccgcccaagatgggggagccggcgggcggccgctacgagcacccgggcttgggcgccctgggcacgcagcagccgccggtcgccgtgcccgggggcggcggcggcccggcggccgtcccggagtttaataattactatggcagcgctgcccctgcgagcggcggccccggcggccgcgctgggccttgctttgatcaacatggcggacaacaaagccccgggatggggatgatgcactccgcctccgccgccgccgccggggcccccggcagcatggaccccctgcagaactcccacgaagggtaccccaacagccagtgcaaccattatccgggctacagccggcccggcgcgggcggcggcggcggcggcggcggcggaggaggaggaggcagcggaggaggaggaggaggaggaggagcaggagcaggaggagcaggagcgggagctgtggcggcggcggccgcggcggcggcggcagcagcaggaggcggcggcggcggcggctatgggggctcgtccgcggggtacggggtgctgagctccccccggcagcagggcggcggcatgatgatgggccccgggggcggcggggccgcgagcctcagcaaggcggccgccggctcggcggcggggggcttccagcgcttcgccggccagaaccagcacccgtcgggggccaccccgaccctcaatcagctgctcacctcgcccagccccatgatgcggagctacggcggcagctaccccgagtacagcagccccagcgcgccgccgccgccgccgtcgcagccccagtcccaggcggcggcggcgggggcggcggcgggcggccagcaggcggccgcgggcatgggcttgggcaaggacatgggcgcccagtacgccgctgccagcccggcctgggcggccgcgcaacaaaggagtcacccggcgatgagccccggcacccccggaccgaccatgggcagatcccagggcagcccaatggatccaatggtgatgaagagacctcagttgtatggcatgggcagtaaccctcattctcagcctcagcagagcagtccgtacccaggaggttcctatggccctccaggcccacagcggtatccaattggcatccagggtcggactcccggggccatggccggaatgcagtaccctcagcagcagatgccacctcagtatggacagcaaggtgtgagtggttactgccagcagggccaacagccatattacagccagcagccgcagcccccgcacctcccaccccaggcgcagtatctgccgtcccagtcccagcagaggtaccagccgcagcaggacatgtctcaggaaggctatggaactagatctcaacctcctctggcccccggaaaacctaaccatgaagacttgaacttaatacagcaagaaagaccatcaagtttaccagatctgtctggctccattgatgacctccccacgggaacggaagcaactttgagctcagcagtcagtgcatccgggtccacgagcagccaaggggatcagagcaacccggcgcagtcgcctttctccccacatgcgtcccctcatctctccagcatcccggggggcccatctccctctcctgttggctctcctgtaggaagcaaccagtctcgatctggcccaatctctcctgcaagtatcccaggtagtcagatgcctccgcagccacccgggagccagtcagaatccagttcccatcccgccttgagccagtcaccaatgccacaggaaagaggttttatggcaggcacacaaagaaaccctcagatggctcagtatggacctcaacagacaggaccatccatgtcgcctcatccttctcctgggggccagatgcatgctggaatcagtagctttcagcagagtaactcaagtgggacttacggtccacagatgagccagtatggaccacaaggtaactactccagacccccagcgtatagtggggtgcccagtgcaagctacagcggcccagggcccggtatgggtatcagtgccaacaaccagatgcatggacaagggccaagccagccatgtggtgctgtgcccctgggacgaatgccatcagctgggatgcagaacagaccatttcctggaaatatgagcagcatgacccccagttctcctggcatgtctcagcagggagggccaggaatggggccgccaatgccaactgtgaaccgtaaggcacaggaggcagccgcagcagtgatgcaggctgctgcgaactcagcacaaagcaggcaaggcagtttccccggcatgaaccagagtggacttatggcttccagctctccctacagccagcccatgaacaacagctctagcctgatgaacacgcaggcgccgccctacagcatggcgcccgccatggtgaacagctcggcagcatctgtgggtcttgcagatatgatgtctcctggtgaatccaaactgcccctgcctctcaaagcagacggcaaagaagaaggcactccacagcccgagagcaagtcaaagaagtccagctcctccaccactactggggagaagatcacgaaggtgtacgagctggggaatgagccagagagaaagctctgggtcgaccgatacctcaccttcatggaagagagaggctctcctgtctcaagtctgcctgccgtgggcaagaagcccctggacctgttccgactctacgtctgcgtcaaagagatcgggggtttggcccaggttaataaaaacaagaagtggcgtgagctggcaaccaacctaaacgttggcacctcaagcagtgcagcgagctccctgaaaaagcagtatattcagtacctgtttgcctttgagtgcaagatcgaacgtggggaggagcccccgccggaagtcttcagcaccggggacaccaaaaagcagcccaagctccagccgccatctcctgctaactcgggatccttgcaaggcccacagaccccccagtcaactggcagcaattccatggcagaggttccaggtgacctgaagccacctaccccagcctccacccctcacggccagatgactccaatgcaaggtggaagaagcagtacaatcagtgtgcacgacccattctcagatgtgagtgattcatccttcccgaaacggaactccatgactccaaacgccccctaccagcagggcatgagcatgcccgatgtgatgggcaggatgccctatgagcccaacaaggacccctttgggggaatgagaaaagtgcctggaagcagcgagccctttatgacgcaaggacagatgcccaacagcagcatgcaggacatgtacaaccaaagtccctccggagcaatgtctaacctgggcatggggcagcgccagcagtttccctatggagccagttacgaccgaaggcatgaaccttatgggcagcagtatccaggccaaggccctccctcgggacagccgccgtatggagggcaccagcccggcctgtacccacagcagccgaattacaaacgccatatggacggcatgtacgggcccccagccaagcgccacgagggcgacatgtacaacatgcagtacagcagccagcagcaggagatgtacaaccagtatggaggctcctactcgggcccggaccgcaggcccatccagggccagtacccgtatccctacagcagggagaggatgcagggcccggggcagatccagacacacggaatcccgcctcagatgatgggcggcccgctgcagtcgtcctccagtgaggggcctcagcagaatatgtgggcagcacgcaatgatatgccttatccctaccagaacaggcagggccctggcggccctacacaggcgcccccttacccaggcatgaaccgcacagacgatatgatggtacccgatcagaggataaatcatgagagccagtggccttctcacgtcagccagcgtcagccttatatgtcgtcctcagcctccatgcagcccatcacacgcccaccacagccgtcctaccagacgccaccgtcactgccaaatcacatctccagggcgcccagcccagcgtccttccagcgctccctggagaaccgcatgtctccaagcaagtctccttttctgccgtctatgaagatgcagaaggtcatgcccacggtccccacatcccaggtcaccgggccaccaccccaaccacccccaatcagaagggagatcacctttcctcctggctcagtagaagcatcacaaccagtcttgaaacaaaggcgaaagattacctccaaagatatcgttactcctgaggcgtggcgtgtgatgatgtcccttaaatcaggtcttttggctgagagtacgtgggctttggacactattaatattcttctgtatgatgacagcactgttgctactttcaatctctcccagttgtctggatttctcgaacttttagtcgagtactttagaaaatgcctgattgacatttttggaattcttatggaatatgaagtgggagaccccagccaaaaagcacttgatcacaacgcagcaaggaaggatgacagccagtccttggcagacgattctgggaaagaggaggaagatgctgaatgtattgatgacgacgaggaagacgaggaggatgaggaggaagacagcgagaagacagaaagcgatgaaaagagcagcatcgctctgactgccccggacgccgctgcagacccaaaggagaagcccaagcaagccagtaagttcgacaagctgccaataaagatagtcaaaaagaacaacctgtttgttgttgaccgatctgacaagttggggcgtgtgcaggagttcaatagtggccttctgcactggcagctcggcgggggtgacaccaccgagcacattcagactcactttgagagcaagatggaaattcctcctcgcaggcgcccacctccccccttaagctccgcaggtagaaagaaagagcaagaaggcaaaggcgactctgaagagcagcaagagaaaagcatcatagcaaccatcgatgacgtcctctctgctcggccaggggcattgcctgaagacgcaaaccctgggccccagaccgaaagcagtaagtttccctttggtatccagcaagccaaaagtcaccggaacatcaagctgctggaggacgagcccaggagccgagacgagactcctctgtgtaccatcgcgcactggcaggactcgctggctaagcgatgcatctgtgtgtccaatattgtccgtagcttgtcattcgtgcctggcaatgatgccgaaatgtccaaacatccaggcctggtgctgatcctggggaagctgattcttcttcaccacgagcatccagagagaaagcgagcaccgcagacctatgagaaagaggaggatgaggacaagggggtggcctgcagcaaagatgagtggtggtgggactgcctcgaggtcttgagggataacacgttggtcacgttggccaacatttccgggcagctagacttgtctgcttacacggaaagcatctgcttgccaattttggatggcttgctgcactggatggtgtgcccgtctgcagaggcacaagatccctttccaactgtgggacccaactcggtcctgtcgcctcagagacttgtgctggagaccctctgtaaactcagtatccaggacaataatgtggacctgatcttggccactcctccatttagtcgtcaggagaaattctatgctacattagttaggtacgttggggatcgcaaaaacccagtctgtcgagaaatgtccatggcgcttttatcgaaccttgcccaaggggacgcactagcagcaagggccatagctgtgcagaaaggaagcattggaaacttgataagcttcctagaggatggggtcacgatggcccagtaccagcagagccagcacaacctcatgcacatgcagcccccgcccctggaaccacctagcgtagacatgatgtgcagggcggccaaggctttgctagccatggccagagtggacgaaaaccgctcggaattccttttgcacgagggccggttgctggatatctcgatatcagctgtcctgaactctctggttgcatctgtcatctgtgatgtactgtttcagattgggcagttatgacataagtgagaaggcaagcatgtgtgagtgaagattagagggtcacatataactggctgttttctgttcttgtttatccagcgtaggaagaaggaaaagaaaatctttgctcctctgccccattcactatttaccaattgggaattaaagaaataattaatttgaacagttatgaaattaatatttgctgtctgtgtgtataagtacatcctttggggttttttttttctcttttttttaaccaaagttgctgtctagtgcattcaaaggtcactttttgttcttcacagatctttttaatgttctttcccatgttgtattgcatttttgggggaagcaaattgactttaaagaaaaaagttgtggcaaaagatgctaagatgcgaaaatttcaccacactgagtcaaaaaggtgaaaaattatccatttcctatgcgttttactcctcagagaatgaaaaaaactgcatcccatcacccaaagttctgtgcaatagaaatttctacagatacaggtataggggctcaaggaggtatgtcggtcagtagtcaaaactatgaaatgatactggtttctccacaggaatatggttccattaggctgggagcaaaaacaatgttttttaagattgagaatacatacctgacaacgatccggaaactgctcctcaccactcccgtcatgcctgctgtcggcgtttgaccttccacgtgacagttcttcacaattcctttcatcattttttaaatattttttttactgcctatgggctgtgatgtatatagaagttgtacattaaacataccctcatttttttcttttcttttttttttttttttttagtacaaagttttagtttctttttcatgatgtggtaactacgaagtgatggtagatttaaataattttttatttttattttatatattttttcattagggccatatctccaaaaaaagaaagaaaaaatacaaaaaacaaaaacaaaaaaaaaagagggtaatgtacaagtttctgtatgtataaagtcatgctcgatttcaggagagcagctgatcacaatttgcttcatgaatcaaggtgtggaaatggttatatatggattgatttagaaaatggttaccagtacagtcaaaaaagagaaaatgaaaaaaatacaactaaaaggaagaaacacaacttcaaagatttttcagtgatgagaatccacatttgtatttcaagataatgtagtttaaaaaaaaaaaaaagaaaaaaacttgatgtaaattcctccttttcctctggcttaatgaatatcatttattcagtataaaatctttatatgttccacatgttaagaataaatgtacattaaatcttgttaagcactgtgatgggtgttcttgaatactgttctagtttccttaaagtggtttcctagtaatcaagttatttacaagaaataggggaatgcagcagtgtattcacattataaaaccctacatttggaagagacctttaggggttacctactttagagtggggagcaacagtttgattttctcaaattacttagctaattagtctttctttgaagcaattaactctaacgacattgaggtatgatcattttcagtatttatgggaggtggctgctgacccacttgaggtgagatctcagaagcttaactggcctgaaaatgtaacattctgccttttactaactccatcttagtttaatcaaagttcaatctattccttgtttcttctgtgtgcctcagagttattttgcatttagtttactccaccgtgtataatatttatactgtgcaatgttaaaaaagaatctgttatattgtatgtggtgtacatagtgcaaagtgatgatttctatttcagggcatattatggttctcatattccttcctacctggtgcacagtagctttttaatactagtcacttctaatttaaactttctcttcctgggtcattgactgttactgtgtaataatcgatttctttgaaactgctgcataattatgctgttagtggacctctacctcttctcttccctctcccaatcacagtatactcagaatccccagcccctcgcatacattgtgtcggttcacattactcacagtaatatatggaagagttagacaagaacatgcagttacagtcattgtgagacgtgactctccagtgtcacgaggaaaaaaatcatcttttctgcaaacagtctctcatctgtcaactcccacattactgagtcaaacagtcttcttacataacaatgcaaccaaatatatgttgaattaaagacccatttataattctgctttaaatacatctgcttgctaagaacagatttcagtgctccaagcttcaaatatggagatttgtaagagggaattcaatattattctaatttctctcttacagagtacaaataaaaggtgtatacaaactccgaacatatccagtattccaattcctttgtcaatcagaagagtaaaataattaacaaaagactgttgttatggtttgcattgtaaccgatacgcagagtctgaccgttgggcaacaagtttttctatcctgatgcgcaacacagtctctagagactaatccaggaagactttagcctcctttccatattctcacccccgaatcaagatttacagaagcccacgaagaatttacagcctgcttgagatcatcttgcctataaactgagttattgctttgtcctaaaaattagtcggtttttttttttctatgaggcttttcagaaatttacaggatgcccagactttacatgtgtaccaaaaaaaaaaaaaagataaaaaataaaggtgcaaagaaagtttagtattttggaatggtgctataaagttgaa

SEQ ID NO: 21 - protein, 385 aa, Homo sapiens
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 22 - protein, 376 aa, Homo sapiens
MMMMALSKTFGQKPVKFQLEDDGEFYMIGSEVGNYLRMFRGSLYKRYPSLWRRLATVEERKKIVASSHDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 23 - protein, 1590 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDRCNVEKVPSNSQLEIEGNSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 24 - protein, 1572 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 25 - protein, 1647 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 26 - protein, 1614 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 27 - protein, 2285 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 28 - protein, 2068 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 29 - protein, 1902 aa, Homo sapiens
MDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 30 - protein, 2236 aa, Homo sapiens
MAHNAGAAAAAGTHSAKSGGSEAALKEGGSAAALSSSSSSSAAAAAASSSSSSGPGSAMETGLLPNHKLKTVGEAPAAPPHQQHHHHHHAHHHHHHAHHLHHHHALQQQLNQFQQQQQQQQQQQQQQQQQQHPISNNNSLGGAGGGAPQPGPDMEQPQHGGAKDSAAGGQADPPGPPLLSKPGDEDDAPPKMGEPAGGRYEHPGLGALGTQQPPVAVPGGGGGPAAVPEFNNYYGSAAPASGGPGGRAGPCFDQHGGQQSPGMGMMHSASAAAAGAPGSMDPLQNSHEGYPNSQCNHYPGYSRPGAGGGGGGGGGGGGGSGGGGGGGGAGAGGAGAGAVAAAAAAAAAAAGGGGGGGYGGSSAGYGVLSSPRQQGGGMMMGPGGGGAASLSKAAAGSAAGGFQRFAGQNQHPSGATPTLNQLLTSPSPMMRSYGGSYPEYSSPSAPPPPPSQPQSQAAAAGAAAGGQQAAAGMGLGKDMGAQYAAASPAWAAAQQRSHPAMSPGTPGPTMGRSQGSPMDPMVMKRPQLYGMGSNPHSQPQQSSPYPGGSYGPPGPQRYPIGIQGRTPGAMAGMQYPQQQMPPQYGQQGVSGYCQQGQQPYYSQQPQPPHLPPQAQYLPSQSQQRYQPQQDMSQEGYGTRSQPPLAPGKPNHEDLNLIQQERPSSLPDLSGSIDDLPTGTEATLSSAVSASGSTSSQGDQSNPAQSPFSPHASPHLSSIPGGPSPSPVGSPVGSNQSRSGPISPASIPGSQMPPQPPGSQSESSSHPALSQSPMPQERGFMAGTQRNPQMAQYGPQQTGPSMSPHPSPGGQMHAGISSFQQSNSSGTYGPQMSQYGPQGNYSRPPAYSGVPSASYSGPGPGMGISANNQMHGQGPSQPCGAVPLGRMPSAGMQNRPFPGNMSSMTPSSPGMSQQGGPGMGPPMPTVNRKAQEAAAAVMQAAANSAQSRQGSFPGMNQSGLMASSSPYSQPMNNSSSLMNTQAPPYSMAPAMVNSSAASVGLADMMSPGESKLPLPLKADGKEEGTPQPESKSKKSSSSTTTGEKITKVYELGNEPERKLWVDRYLTFMEERGSPVSSLPAVGKKPLDLFRLYVCVKEIGGLAQVNKNKKWRELATNLNVGTSSSAASSLKKQYIQYLFAFECKIERGEEPPPEVFSTGDTKKQPKLQPPSPANSGSLQGPQTPQSTGSNSMAEVPGDLKPPTPASTPHGQMTPMQGGRSSTISVHDPFSDVSDSSFPKRNSMTPNAPYQQGMSMPDVMGRMPYEPNKDPFGGMRKVPGSSEPFMTQGQMPNSSMQDMYNQSPSGAMSNLGMGQRQQFPYGASYDRRHEPYGQQYPGQGPPSGQPPYGGHQPGLYPQQPNYKRHMDGMYGPPAKRHEGDMYNMQYSSQQQEMYNQYGGSYSGPDRRPIQGQYPYPYSRERMQGPGQIQTHGIPPQMMGGPLQSSSSEGPQQNMWAARNDMPYPYQNRQGPGGPTQAPPYPGMNRTDDMMVPDQRINHESQWPSHVSQRQPYMSSSASMQPITRPPQPSYQTPPSLPNHISRAPSPASFQRSLENRMSPSKSPFLPSMKMQKVMPTVPTSQVTGPPPQPPPIRREITFPPGSVEASQPVLKQRRKITSKDIVTPEAWRVMMSLKSGLLAESTWALDTINILLYDDSTVATFNLSQLSGFLELLVEYFRKCLIDIFGILMEYEVGDPSQKALDHNAARKDDSQSLADDSGKEEEDAECIDDDEEDEEDEEEDSEKTESDEKSSIALTAPDAAADPKEKPKQASKFDKLPIKIVKKNNLFVVDRSDKLGRVQEFNSGLLHWQLGGGDTTEHIQTHFESKMEIPPRRRPPPPLSSAGRKKEQEGKGDSEEQQEKSIIATIDDVLSARPGALPEDANPGPQTESSKFPFGIQQAKSHRNIKLLEDEPRSRDETPLCTIAHWQDSLAKRCICVSNIVRSLSFVPGNDAEMSKHPGLVLILGKLILLHHEHPERKRAPQTYEKEEDEDKGVACSKDEWWWDCLEVLRDNTLVTLANISGQLDLSAYTESICLPILDGLLHWMVCPSAEAQDPFPTVGPNSVLSPQRLVLETLCKLSIQDNNVDLILATPPFSRQEKFYATLVRYVGDRKNPVCREMSMALLSNLAQGDALAARAIAVQKGSIGNLISFLEDGVTMAQYQQSQHNLMHMQPPPLEPPSVDMMCRAAKALLAMARVDENRSEFLLHEGRLLDISISAVLNSLVASVICDVLFQIGQL

SEQ ID NO: 31 - protein, 2249 aa, Homo sapiens
MAHNAGAAAAAGTHSAKSGGSEAALKEGGSAAALSSSSSSSAAAAAASSSSSSGPGSAMETGLLPNHKLKTVGEAPAAPPHQQHHHHHHAHHHHHHAHHLHHHHALQQQLNQFQQQQQQQQQQQQQQQQQQHPISNNNSLGGAGGGAPQPGPDMEQPQHGGAKDSAAGGQADPPGPPLLSKPGDEDDAPPKMGEPAGGRYEHPGLGALGTQQPPVAVPGGGGGPAAVPEFNNYYGSAAPASGGPGGRAGPCFDQHGGQQSPGMGMMHSASAAAAGAPGSMDPLQNSHEGYPNSQCNHYPGYSRPGAGGGGGGGGGGGGGSGGGGGGGGAGAGGAGAGAVAAAAAAAAAAAGGGGGGGYGGSSAGYGVLSSPRQQGGGMMMGPGGGGAASLSKAAAGSAAGGFQRFAGQNQHPSGATPTLNQLLTSPSPMMRSYGGSYPEYSSPSAPPPPPSQPQSQAAAAGAAAGGQQAAAGMGLGKDMGAQYAAASPAWAAAQQRSHPAMSPGTPGPTMGRSQGSPMDPMVMKRPQLYGMGSNPHSQPQQSSPYPGGSYGPPGPQRYPIGIQGRTPGAMAGMQYPQQQDSGDATWKETFWLMPPQYGQQGVSGYCQQGQQPYYSQQPQPPHLPPQAQYLPSQSQQRYQPQQDMSQEGYGTRSQPPLAPGKPNHEDLNLIQQERPSSLPDLSGSIDDLPTGTEATLSSAVSASGSTSSQGDQSNPAQSPFSPHASPHLSSIPGGPSPSPVGSPVGSNQSRSGPISPASIPGSQMPPQPPGSQSESSSHPALSQSPMPQERGFMAGTQRNPQMAQYGPQQTGPSMSPHPSPGGQMHAGISSFQQSNSSGTYGPQMSQYGPQGNYSRPPAYSGVPSASYSGPGPGMGISANNQMHGQGPSQPCGAVPLGRMPSAGMQNRPFPGNMSSMTPSSPGMSQQGGPGMGPPMPTVNRKAQEAAAAVMQAAANSAQSRQGSFPGMNQSGLMASSSPYSQPMNNSSSLMNTQAPPYSMAPAMVNSSAASVGLADMMSPGESKLPLPLKADGKEEGTPQPESKSKKSSSSTTTGEKITKVYELGNEPERKLWVDRYLTFMEERGSPVSSLPAVGKKPLDLFRLYVCVKEIGGLAQVNKNKKWRELATNLNVGTSSSAASSLKKQYIQYLFAFECKIERGEEPPPEVFSTGDTKKQPKLQPPSPANSGSLQGPQTPQSTGSNSMAEVPGDLKPPTPASTPHGQMTPMQGGRSSTISVHDPFSDVSDSSFPKRNSMTPNAPYQQGMSMPDVMGRMPYEPNKDPFGGMRKVPGSSEPFMTQGQMPNSSMQDMYNQSPSGAMSNLGMGQRQQFPYGASYDRRHEPYGQQYPGQGPPSGQPPYGGHQPGLYPQQPNYKRHMDGMYGPPAKRHEGDMYNMQYSSQQQEMYNQYGGSYSGPDRRPIQGQYPYPYSRERMQGPGQIQTHGIPPQMMGGPLQSSSSEGPQQNMWAARNDMPYPYQNRQGPGGPTQAPPYPGMNRTDDMMVPDQRINHESQWPSHVSQRQPYMSSSASMQPITRPPQPSYQTPPSLPNHISRAPSPASFQRSLENRMSPSKSPFLPSMKMQKVMPTVPTSQVTGPPPQPPPIRREITFPPGSVEASQPVLKQRRKITSKDIVTPEAWRVMMSLKSGLLAESTWALDTINILLYDDSTVATFNLSQLSGFLELLVEYFRKCLIDIFGILMEYEVGDPSQKALDHNAARKDDSQSLADDSGKEEEDAECIDDDEEDEEDEEEDSEKTESDEKSSIALTAPDAAADPKEKPKQASKFDKLPIKIVKKNNLFVVDRSDKLGRVQEFNSGLLHWQLGGGDTTEHIQTHFESKMEIPPRRRPPPPLSSAGRKKEQEGKGDSEEQQEKSIIATIDDVLSARPGALPEDANPGPQTESSKFPFGIQQAKSHRNIKLLEDEPRSRDETPLCTIAHWQDSLAKRCICVSNIVRSLSFVPGNDAEMSKHPGLVLILGKLILLHHEHPERKRAPQTYEKEEDEDKGVACSKDEWWWDCLEVLRDNTLVTLANISGQLDLSAYTESICLPILDGLLHWMVCPSAEAQDPFPTVGPNSVLSPQRLVLETLCKLSIQDNNVDLILATPPFSRQEKFYATLVRYVGDRKNPVCREMSMALLSNLAQGDALAARAIAVQKGSIGNLISFLEDGVTMAQYQQSQHNLMHMQPPPLEPPSVDMMCRAAKALLAMARVDENRSEFLLHEGRLLDISISAVLNSLVASVICDVLFQIGQL

SEQ ID NO: 32 - protein, 2289 aa, Homo sapiens
MAHNAGAAAAAGTHSAKSGGSEAALKEGGSAAALSSSSSSSAAAAAASSSSSSGPGSAMETGLLPNHKLKTVGEAPAAPPHQQHHHHHHAHHHHHHAHHLHHHHALQQQLNQFQQQQQQQQQQQQQQQQQQHPISNNNSLGGAGGGAPQPGPDMEQPQHGGAKDSAAGGQADPPGPPLLSKPGDEDDAPPKMGEPAGGRYEHPGLGALGTQQPPVAVPGGGGGPAAVPEFNNYYGSAAPASGGPGGRAGPCFDQHGGQQSPGMGMMHSASAAAAGAPGSMDPLQNSHEGYPNSQCNHYPGYSRPGAGGGGGGGGGGGGGSGGGGGGGGAGAGGAGAGAVAAAAAAAAAAAGGGGGGGYGGSSAGYGVLSSPRQQGGGMMMGPGGGGAASLSKAAAGSAAGGFQRFAGQNQHPSGATPTLNQLLTSPSPMMRSYGGSYPEYSSPSAPPPPPSQPQSQAAAAGAAAGGQQAAAGMGLGKDMGAQYAAASPAWAAAQQRSHPAMSPGTPGPTMGRSQGSPMDPMVMKRPQLYGMGSNPHSQPQQSSPYPGGSYGPPGPQRYPIGIQGRTPGAMAGMQYPQQQMPPQYGQQGVSGYCQQGQQPYYSQQPQPPHLPPQAQYLPSQSQQRYQPQQDMSQEGYGTRSQPPLAPGKPNHEDLNLIQQERPSSLPDLSGSIDDLPTGTEATLSSAVSASGSTSSQGDQSNPAQSPFSPHASPHLSSIPGGPSPSPVGSPVGSNQSRSGPISPASIPGSQMPPQPPGSQSESSSHPALSQSPMPQERGFMAGTQRNPQMAQYGPQQTGPSMSPHPSPGGQMHAGISSFQQSNSSGTYGPQMSQYGPQGNYSRPPAYSGVPSASYSGPGPGMGISANNQMHGQGPSQPCGAVPLGRMPSAGMQNRPFPGNMSSMTPSSPGMSQQGGPGMGPPMPTVNRKAQEAAAAVMQAAANSAQSRQGSFPGMNQSGLMASSSPYSQPMNNSSSLMNTQAPPYSMAPAMVNSSAASVGLADMMSPGESKLPLPLKADGKEEGTPQPESKSKDSYSSQGISQPPTPGNLPVPSPMSPSSASISSFHGDESDSISSPGWPKTPSSPKSSSSTTTGEKITKVYELGNEPERKLWVDRYLTFMEERGSPVSSLPAVGKKPLDLFRLYVCVKEIGGLAQVNKNKKWRELATNLNVGTSSSAASSLKKQYIQYLFAFECKIERGEEPPPEVFSTGDTKKQPKLQPPSPANSGSLQGPQTPQSTGSNSMAEVPGDLKPPTPASTPHGQMTPMQGGRSSTISVHDPFSDVSDSSFPKRNSMTPNAPYQQGMSMPDVMGRMPYEPNKDPFGGMRKVPGSSEPFMTQGQMPNSSMQDMYNQSPSGAMSNLGMGQRQQFPYGASYDRRHEPYGQQYPGQGPPSGQPPYGGHQPGLYPQQPNYKRHMDGMYGPPAKRHEGDMYNMQYSSQQQEMYNQYGGSYSGPDRRPIQGQYPYPYSRERMQGPGQIQTHGIPPQMMGGPLQSSSSEGPQQNMWAARNDMPYPYQNRQGPGGPTQAPPYPGMNRTDDMMVPDQRINHESQWPSHVSQRQPYMSSSASMQPITRPPQPSYQTPPSLPNHISRAPSPASFQRSLENRMSPSKSPFLPSMKMQKVMPTVPTSQVTGPPPQPPPIRREITFPPGSVEASQPVLKQRRKITSKDIVTPEAWRVMMSLKSGLLAESTWALDTINILLYDDSTVATFNLSQLSGFLELLVEYFRKCLIDIFGILMEYEVGDPSQKALDHNAARKDDSQSLADDSGKEEEDAECIDDDEEDEEDEEEDSEKTESDEKSSIALTAPDAAADPKEKPKQASKFDKLPIKIVKKNNLFVVDRSDKLGRVQEFNSGLLHWQLGGGDTTEHIQTHFESKMEIPPRRRPPPPLSSAGRKKEQEGKGDSEEQQEKSIIATIDDVLSARPGALPEDANPGPQTESSKFPFGIQQAKSHRNIKLLEDEPRSRDETPLCTIAHWQDSLAKRCICVSNIVRSLSFVPGNDAEMSKHPGLVLILGKLILLHHEHPERKRAPQTYEKEEDEDKGVACSKDEWWWDCLEVLRDNTLVTLANISGQLDLSAYTESICLPILDGLLHWMVCPSAEAQDPFPTVGPNSVLSPQRLVLETLCKLSIQDNNVDLILATPPFSRQEKFYATLVRYVGDRKNPVCREMSMALLSNLAQGDALAARAIAVQKGSIGNLISFLEDGVTMAQYQQSQHNLMHMQPPPLEPPSVDMMCRAAKALLAMARVDENRSEFLLHEGRLLDISISAVLNSLVASVICDVLFQIGQL

SEQ ID NO: 33 - protein, 1486 aa, Homo sapiens
MPPQPPGSQSESSSHPALSQSPMPQERGFMAGTQRNPQMAQYGPQQTGPSMSPHPSPGGQMHAGISSFQQSNSSGTYGPQMSQYGPQGNYSRPPAYSGVPSASYSGPGPGMGISANNQMHGQGPSQPCGAVPLGRMPSAGMQNRPFPGNMSSMTPSSPGMSQQGGPGMGPPMPTVNRKAQEAAAAVMQAAANSAQSRQGSFPGMNQSGLMASSSPYSQPMNNSSSLMNTQAPPYSMAPAMVNSSAASVGLADMMSPGESKLPLPLKADGKEEGTPQPESKSKKSSSSTTTGEKITKVYELGNEPERKLWVDRYLTFMEERGSPVSSLPAVGKKPLDLFRLYVCVKEIGGLAQVNKNKKWRELATNLNVGTSSSAASSLKKQYIQYLFAFECKIERGEEPPPEVFSTGDTKKQPKLQPPSPANSGSLQGPQTPQSTGSNSMAEVPGDLKPPTPASTPHGQMTPMQGGRSSTISVHDPFSDVSDSSFPKRNSMTPNAPYQQGMSMPDVMGRMPYEPNKDPFGGMRKVPGSSEPFMTQGQMPNSSMQDMYNQSPSGAMSNLGMGQRQQFPYGASYDRRHEPYGQQYPGQGPPSGQPPYGGHQPGLYPQQPNYKRHMDGMYGPPAKRHEGDMYNMQYSSQQQEMYNQYGGSYSGPDRRPIQGQYPYPYSRERMQGPGQIQTHGIPPQMMGGPLQSSSSEGPQQNMWAARNDMPYPYQNRQGPGGPTQAPPYPGMNRTDDMMVPDQRINHESQWPSHVSQRQPYMSSSASMQPITRPPQPSYQTPPSLPNHISRAPSPASFQRSLENRMSPSKSPFLPSMKMQKVMPTVPTSQVTGPPPQPPPIRREITFPPGSVEASQPVLKQRRKITSKDIVTPEAWRVMMSLKSGLLAESTWALDTINILLYDDSTVATFNLSQLSGFLELLVEYFRKCLIDIFGILMEYEVGDPSQKALDHNAARKDDSQSLADDSGKEEEDAECIDDDEEDEEDEEEDSEKTESDEKSSIALTAPDAAADPKEKPKQASKFDKLPIKIVKKNNLFVVDRSDKLGRVQEFNSGLLHWQLGGGDTTEHIQTHFESKMEIPPRRRPPPPLSSAGRKKEQEGKGDSEEQQEKSIIATIDDVLSARPGALPEDANPGPQTESSKFPFGIQQAKSHRNIKLLEDEPRSRDETPLCTIAHWQDSLAKRCICVSNIVRSLSFVPGNDAEMSKHPGLVLILGKLILLHHEHPERKRAPQTYEKEEDEDKGVACSKDEWWWDCLEVLRDNTLVTLANISGQLDLSAYTESICLPILDGLLHWMVCPSAEAQDPFPTVGPNSVLSPQRLVLETLCKLSIQDNNVDLILATPPFSRQEKFYATLVRYVGDRKNPVCREMSMALLSNLAQGDALAARAIAVQKGSIGNLISFLEDGVTMAQYQQSQHNLMHMQPPPLEPPSVDMMCRAAKALLAMARVDENRSEFLLHEGRLLDISISAVLNSLVASVICDVLFQIGQL

SEQ ID NO: 34 - protein, 418 aa, Homo sapiens
MSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 35 - protein, 387 aa, Homo sapiens
MSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ